(19) United States
(12) Patent Application Publication
(10) Pub. No.: US 2026/0226071 A1
(43) Pub. Date: Aug. 6, 2026

(54) 2, 4-DIANILINOPYRIMIDINE-BASED AURORA-A KINASE SELECTIVE DEGRADATION INDUCING COMPOUNDS

(71) Applicant: UPPTHERA, INC., Incheon (KR)

(72) Inventors: Soo Hee Ryu, Incheon (KR); Seong Hoon Kim, Incheon (KR); Han Kyu Lee, Incheon (KR); Ha Na Jeong, Incheon (KR)

(73) Assignee: UPPTHERA, INC., Incheon (KR)

(21) Appl. No.: 19/147,914

(22) PCT Filed: Jan. 17, 2024
(86) PCT No.: PCT/KR2024/000878

§ 371 Date: Jul. 14, 2025

(30) Foreign Application Priority Data
Jan. 18, 2023 (KR) 10-2023-0007668
Sep. 1, 2023 (KR) 10-2023-0116087

(51) Int. Cl. C07D 487/10; A61K 31/506; A61K 31/513; A61K 31/551; A61K 47/54; A61K 47/55; C07D 401/12; C07D 401/14; C07D 471/10; C07D 487/04; C07D 487/08

(52) U.S. Cl. CPC C07D 487/10; A61K 31/506; A61K 31/513; A61K 31/551; A61K 47/545; A61K 47/55; C07D 401/12; C07D 401/14; C07D 471/10; C07D 487/04; C07D 487/08

(57) ABSTRACT

Novel 2,4-Dianilinopyrimidine-based Aurora-A kinase (AURKA) degraders are disclosed. The present AURKA degraders are proteolysis targeting chimeras (PROTACs) that recruit AURKA protein into optimized CRBN E3 ubiquitin ligase via linkers. The compounds may induce selective AURKA degradation and may be utilized for the treatment of cancer.

### TECHNICAL FIELD

The present disclosure relates to a novel Aurora-A kinase selective degradation inducing compound with modified cereblon binders, a method for preparing the same, and the use thereof.

### BACKGROUND ART

Aurora kinases belong to serine/threonine kinase family that are essential for cell proliferation. The two major Aurora kinases, Aurora-A and Aurora-B are closely related in kinase domain sequence but have quite different functions during mitosis. Aurora-A kinase (AURKA) selective inhibitors, among small molecule-based aurora kinase inhibitors, have been extensively investigated as potential anticancer therapeutic agents (Borisa, Ankit C., and Hardik G. Bhatt. “A comprehensive review on Aurora kinase: Small molecule inhibitors and clinical trial studies.” European journal of medicinal chemistry 140 (2017): 1-19.).

Although the catalytic activity of AURKA is essential for its mitotic function, recent reports indicate an additional non-catalytic function, which is difficult to target by conventional small molecules. As an alternative approach, Proteolysis targeting chimeras (PROTACs)-mediated AURKA depletion implied an important non-catalytic function of AURKA during DNA replication (Adhikari, Bikash, et al. “PROTAC-mediated degradation reveals a non-catalytic function of AURORA-A kinase.” Nature chemical biology 16.11 (2020): 1179-1188.).

2,4-dianilinopyrimidine derivatives have been reported as excellent selective AURKA inhibitors (Aliagas-Martin, Ignacio, et al. “A class of 2, 4-dianilinopyrimidine Aurora A inhibitors with unusually high selectivity against Aurora B.” Journal of medicinal chemistry 52.10 (2009): 3300-3307.). The present applicant previously reported PROTAC-mediated AURKA selective degradation, by synthesis of novel PROTAC compounds by connecting a 2,4-dianilinopyrimidine derivative to the von Hippel-Lindau (VHL) or cereblon (CRBN) binders (e.g., thalidomide) via a linker.

In the current PROTACs technology, modulating the chemical composition of CRBN binders is a critical step in the optimization process of protein degraders and small structural changes can have profound impacts on the overall profile of PROTACs, including depth of on-target degradation, off-target effects, as well as other drug-like properties (Norris, Stephen, et al. “Design and Synthesis of Novel Cereblon Binders for Use in Targeted Protein Degradation.” Journal of Medicinal Chemistry (2023).). Under the circumstances, by using glutarimide based CRBN binders with lower molecular weight, the inventors have succeeded in synthesis of novel optimized AURKA degraders with efficacious AURKA selective degradation and/or cytotoxicity over cancer cells as well as improved drug properties.

### DISCLOSURE OF INVENTION

### Technical Problem

An objective of the invention is to provide novel selective AURKA degraders, as well as the preparation methods and the use thereof.

### Solution to Problem

To solve the problem, the present disclosure provides novel compounds defined as Formula I, as well as the preparation methods and the use thereof.

### Design of Novel Aurora Kinase A Degraders

In one general aspect, the present disclosure provides a novel compound represented by the following Formula I:

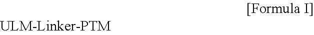

wherein:
PTM is Aurora kinase A (AURKA) binding moiety represented by the following Formula II:

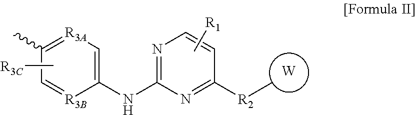

wherein:
R₁ is halo;
R₂ is —NH— or —O—;
R_{3A} and R_{3B} are each independently —CH— or —N—;
R_{3C} is H, halo or OC₁₋₃alkyl;

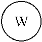

is

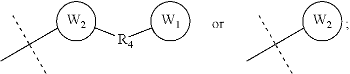

R₄ is —CO—, —CONH—, —CON(CH₃)— or —NHCO—;

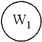

is 5- to 6-membered cycloalkyl, heterocyclyl, aryl or heteroaryl optionally substituted with 1 to 3 halo;

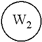

is phenyl or 5- to 6-membered heteroaryl optionally substituted with halo or C₁₋₃alkyl; and
[symbol] is a covalent bond connected to Linker; and

ULM is CRBN E3 ubiquitin ligase binding moiety represented by the following Formula III-1, III-2A or III-2B:

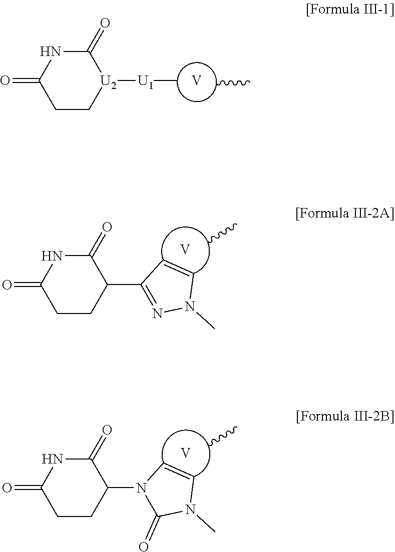

wherein:
U₁ is a covalent bond, —NH—, —N(CH₃)—, —NHCO— or —CONH—;
U₂ is CH or N;

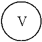

is phenyl or 6-membered heteroaryl optionally substituted with 1 to 3 halo, C₁₋₃alkyl or OC₁₋₃alkyl; and
[symbol] is a covalent bond connected to Linker; and

Linker is a linking group represented by the following Formula IV:

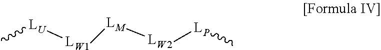

wherein:
L_{U} is a covalent bond, —CH₂—, —NH—, —O—, CH₂CH₂—, —NHCH₂—, —CH₂NH—, —OCH₂—, —CH₂O—, —CH═CH— or —CC—;
L_{M} is a covalent bond, —CH₂—, —CH₂CH₂—, —CH(CH₃)CH₂—, —CH₂CH(CH₃)—, —CH(CH₂CH₃)CH₂—, —CH₂CH(CH₂CH₃)—, —COCH₂—, —CH₂CO—, —CH₂NH—, —NHCH₂—, —CH₂N(CH₃)—, —N(CH₃)CH₂—, —CH₂CH₂CH₂—, —COCH₂CH₂—, —CH₂COCH₂—, —CH₂CH₂CO—, —NHCH₂CH₂—, —CH₂NHCH₂—, —CH₂CH₂NH—, —N(CH₃)CH₂CH₂—, —CH₂N(CH₃)CH₂— or —CH₂CH₂N(CH₃);
L_{P} is —CH₂—, —O—, —CH₂O—, —OCH₂—, —NH—, —CH₂NH—, —NHCH₂—, —N(CH₃)CH₂—, —CH₂N(CH₃)—, —CO—, —COCH₂—, —NHCO—, —CH₂NHCO—, —CONHCH₂—, —NHCOCH₂—, —CONH— or —CH₂CONH—;
L_{W1} and L_{W2} are each independently 3- to 11-membered cycloalkyl, heterocyclyl, aryl or heteroaryl optionally substituted with 1 to 3 C₁₋₃alkyl or OH; and
L_{U} and L_{P} are connected to ULM and PTM through [symbol] respectively.

The novel compounds represented by Formula I above are Proteolysis targeting chimeras (PROTACs) that are bispecific molecules containing a target protein binder and an E3 ubiquitin ligase binder connected by a linker. By recruiting CRBN E3 ubiquitin ligase to a target protein AURKA, the PROTACs may promote ubiquitination and eventually proteasomal degradation of the target protein. (FIG. 1).

### (1) Aurora Kinase A Binding Moiety (PTM)

In Formula I, PTM represented by Formula II is a 2,4-dianilinopyrimidine-based moiety with selective Aurora kinase A (AURKA) binding affinity.

Aurora kinase A (AURKA) is a serine/threonine protein kinase that in humans is encoded by the AURKA gene. (e.g., NCBI Reference Sequence: NP_001310234.1). AURKA is implicated with important processes during mitosis and meiosis whose proper function is integral for healthy cell proliferation. AURKA is activated by one or more phosphorylations and its activity peaks during the G2 phase to M phase transition in the cell cycle. AURKA shows significantly higher expression in cancer tissues than in normal control tissues for multiple tumor types. Activation of AURKA has been demonstrated to play an important role in a wide range of cancers.

In one embodiment, Formula II is the following Formula II-1:

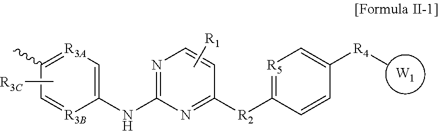

wherein:
R₁ is halo;
R₂ is —NH— or —O—;
R_{3A} and R_{3B} are each independently —CH— or —N—;
R_{3C} is H, halo or —OC₁₋₃alkyl;
R₄ is —CONH—, —CON(CH₃)— or —NHCO—;
R₅ is CH or N;

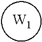

is cyclohexyl or phenyl optionally substituted with 1 to 3 halo; and
[symbol] is a covalent bond connected to Linker.

In one embodiment, Formula II-1 is one of the following Formula II-1A, II-1B, II-1C, II-1D or II-1E:

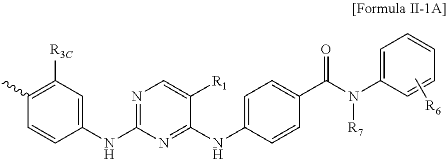

wherein R₁ is halo; R_{3C} is H, halo or OCH₃; R₆ is H or halo; and R₇ is H or CH₃.

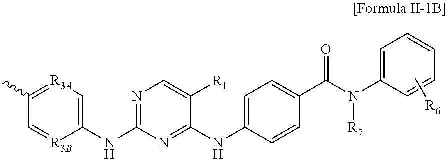

wherein R₁ is halo; R_{3A} and R_{3B} are each independently CH or N; and R₆ is H or halo.

In one embodiment, one of R_{3A} and R_{3B} in Formula II-1B is N; and the other is CH.

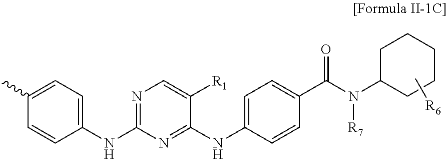

wherein R₁ is halo; R₆ is H or halo; and R₇ is H or CH₃.

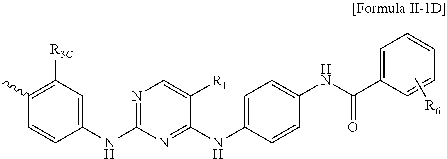

wherein R₁ is halo; R_{3C} is H or halo; and R₆ is H or halo.

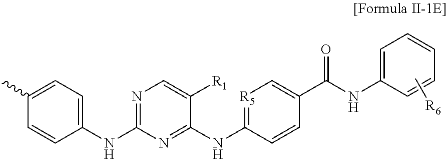

wherein R₁ is halo; R₂ is —NH— or —O—; R₅ is CH or N; and R₆ is H or halo.

In one embodiment, R₆ in Formula II-1A, II-1B, II-1C, II-1D or II-1E is connected to its parent ring by ortho-, meta- or para-position, preferably by ortho-position.

In one embodiment, Formula II is the following Formula II-2:

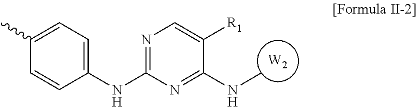

wherein:
R₁ is halo; and

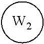

is phenyl or pyrazolyl optionally substituted with halo or C₁₋₃alkyl.

In one embodiment, Formula II-2 is the following Formula II-2A or II-2B:

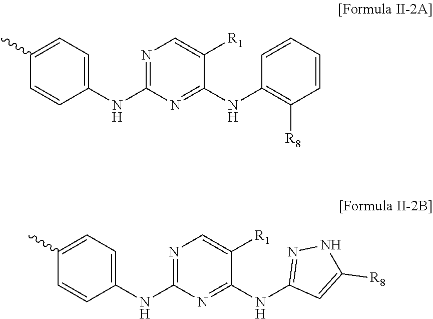

wherein R₈ is H, halo or C₁₋₃alkyl.

### (2) Cereblon E3 Ubiquitin Ligase Binding Moiety (ULM)

In Formula I, ULM represented by Formula III-1, III-2A or III-2B is an optimized cereblon binder in which a glutarimide moiety is conjugated to aryl or heteroaryl ring directly or mediated by a short linking group.

Cereblon (CRBN) is a subunit of E3 ubiquitin ligase protein that in humans is encoded by the CRBN gene. CRBN forms an E3 ubiquitin ligase complex with damaged DNA binding protein 1 (DDB1), Cullin-4A (CUL4A), and regulator of cullins 1 (ROC1), leading to ubiquitination of substrate proteins.

In one embodiment, Formula III-1 is the following Formula III-1A:

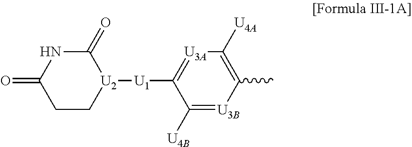

wherein:
U₁ is a covalent bond, —NH—, —N(CH₃)—, —NHCO— or —CONH—;
U₂ is CH or N;
U_{3A} and U_{3B} are each independently CH or N; and
U_{4A} and U_{4B} are each independently H, halo, C₁₋₃alkyl or OC₁₋₃alkyl.

In one embodiment, Formula III-1A is the following Formula:

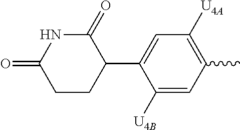

wherein U_{4A} and U_{4B} are each independently H, halo, C₁₋₃alkyl or OC₁₋₃alkyl.

In one embodiment, Formula III-1A is the following Formula:

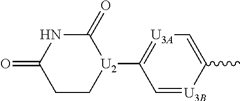

wherein at least one of U₂, U_{3A} and U_{3B} is N; and the others are CH.

In one embodiment, Formula III-1A is the following Formula:

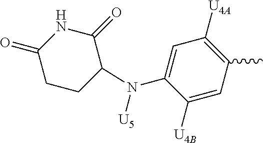

wherein U_{4A} and U_{4B} are each independently H, halo, C₁₋₃alkyl or OC₁₋₃alkyl; and U₅ is H or CH₃.

In one embodiment, Formula III-1A is the following Formula:

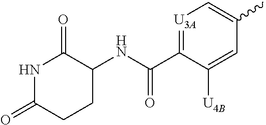

wherein U_{3A} is CH or N; and U_{4B} is H, halo, C₁₋₃alkyl or OC₁₋₃alkyl.

In one embodiment, Formula III-1 is the following Formula III-1B:

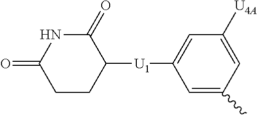

wherein:
U₁ is a covalent bond, —NH—, —N(CH₃)—, —NHCO— or —CONH—; and
U_{4A} is H, halo, C₁₋₃alkyl or OC₁₋₃alkyl.

In one embodiment, Formula III-2A is the following Formula III-2C:

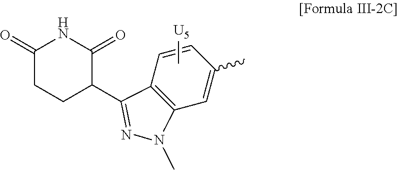

wherein U₅ is H or halo.

In one embodiment, Formula III-2B is the following Formula III-2D:

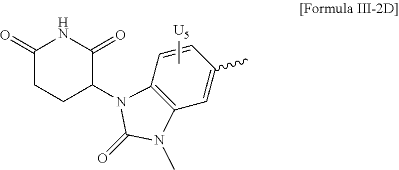

wherein U₅ is H or halo.

### (3) Linker

In Formula I, Linker represented by Formula IV is a linking group that connects ULM and PTM, thereby leading AURKA and CRBN E3 ubiquitin ligase being in close proximity, which are recruited by PTM and ULM respectively.

In one embodiment, Linker represented by Formula IV is a linking group that connects ULM and PTM by 4 to 20 atoms in shortest lengths, preferably by 8 to 14 atoms.

In Formula IV, L_{W1} and L_{W2} may be each independently Formula IV-1:

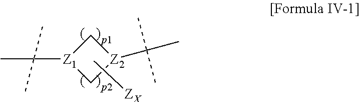

wherein:
Z₁ and Z₂ are each independently CH or N;
Z_{X} is H, CH₃ or OH;
p₁ is 0, 1 or 2; and
p₂ is 1, 2 or 3;

In one embodiment, Formula IV-1 is one of the following groups LW-1 to 12:

TABLE 1

| | Structure |
|---|---|
| LW-1 | 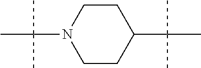 |
| LW-2 | 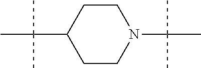 |
| LW-3 | 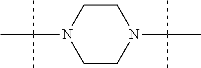 |
| LW-4 | 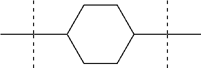 |
| LW-5 | 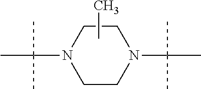 |
| LW-6 | 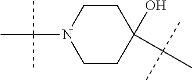 |
| LW-7 | 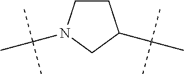 |
| LW-8 | 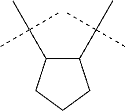 |
| LW-9 | 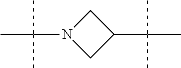 |
| LW-10 | 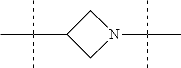 |
| LW-11 | 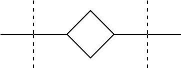 |
| LW-12 | 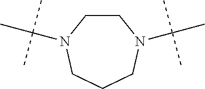 |

In Formula IV, L_{W1} and L_{W2} may be each independently Formula IV-2:

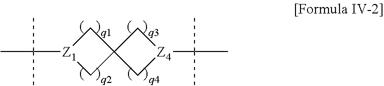

wherein:
Z₃ and Z₄ are each independently CH or N;
q₁ and q₃ are each independently 0, 1 or 2; and
q₂ and q₄ are each independently 1 or 2.

In one embodiment, Formula IV-2 is one of the following groups LW-13 to 19:

TABLE 2

| | Structure |
|---|---|
| LW-13 | 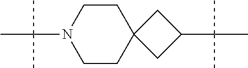 |
| LW-14 | 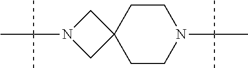 |
| LW-15 | 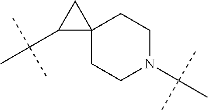 |
| LW-16 | 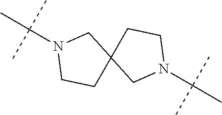 |
| LW-17 | 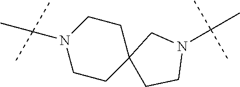 |
| LW-18 | 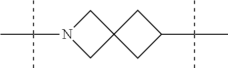 |
| LW-19 | 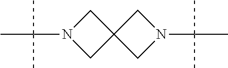 |

In Formula IV, L_{W1} and L_{W2} may be each independently Formula IV-3:

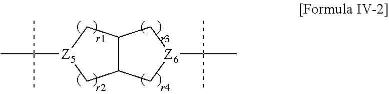

wherein:
Z₅ and Z₆ are each independently CH or N; and
r₁ to r₄ are each independently 0 or 1.

In one embodiment, Formula IV-2 is one of the following groups LW-20 to 23:

TABLE 3

| | Structure |
|---|---|
| LW-20 | 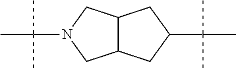 |
| LW-21 | 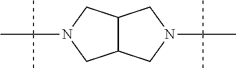 |
| LW-22 | 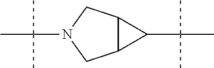 |
| LW-23 | 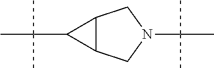 |

In Formula IV, L_{W1} and L_{W2} may be each independently one of the following groups:

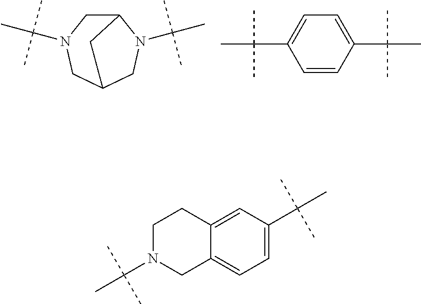

In one embodiment, Formula IV is selected from the Linker-1 to Linker-68 in Table 4, wherein [symbol] and [symbol] are covalent bonds which are connected to ULM and PTM, respectively:

TABLE 4

| Linker | Structure |
|---|---|
| 1 | 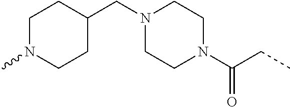 |
| 2 | 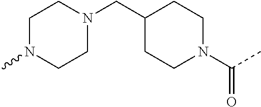 |
| 3 | 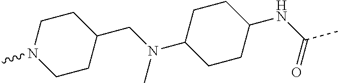 |
| 4 | 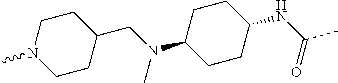 |
| 5 | 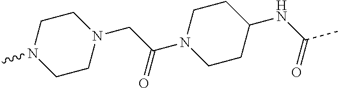 |
| 6 | 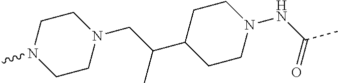 |
| 7 | 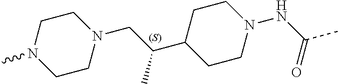 |
| 8 | 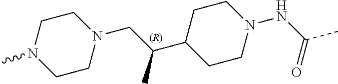 |
| 9 | 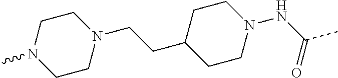 |
| 10 | 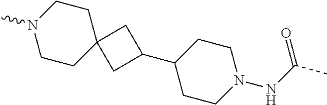 |
| 11 | 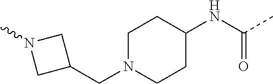 |
| 12 | 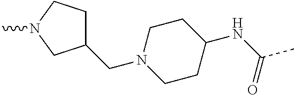 |
| 13 | 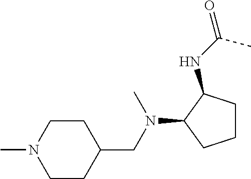 |
| 14 | 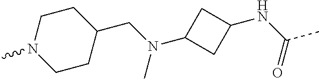 |
| 15 | 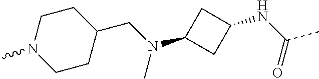 |
| 16 | 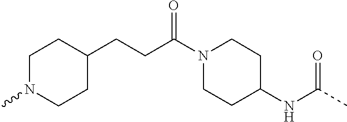 |
| 17 | 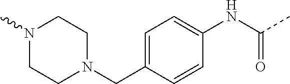 |
| 18 | 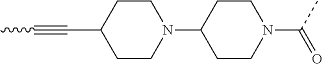 |
| 19 | 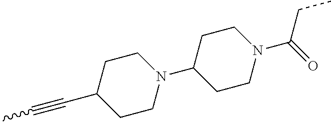 |
| 20 | 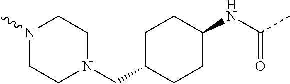 |
| 21 | 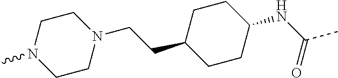 |
| 22 | 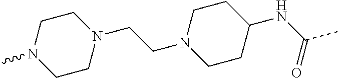 |
| 23 | 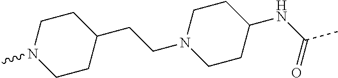 |
| 24 | 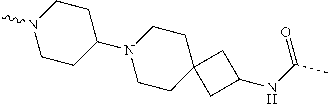 |
| 25 | 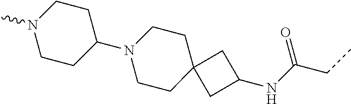 |
| 26 | 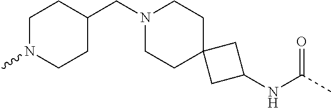 |
| 27 | 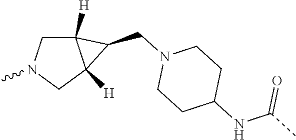 |
| 28 | 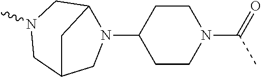 |
| 29 | 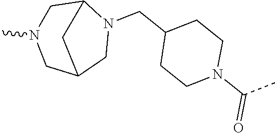 |
| 30 | 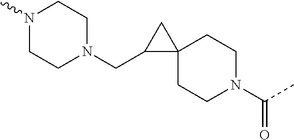 |
| 31 | 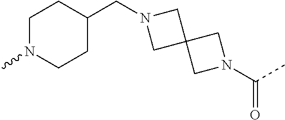 |
| 32 | 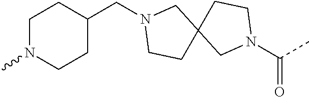 |
| 33 | 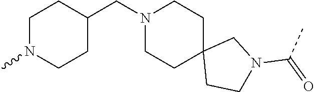 |
| 34 | 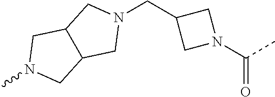 |
| 35 | 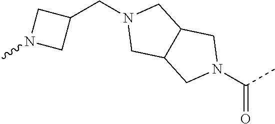 |
| 36 | 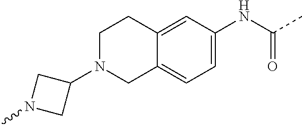 |
| 37 | 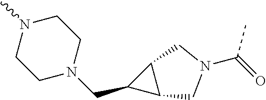 |
| 38 | 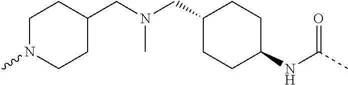 |
| 39 | 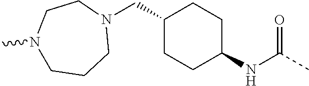 |
| 40 | 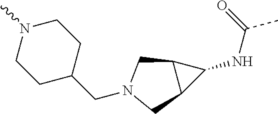 |
| 41 | 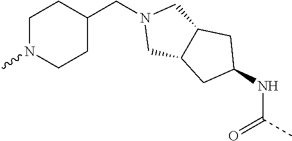 |
| 42 | 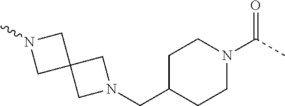 |
| 43 | 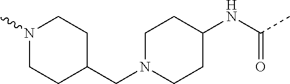 |
| 44 | 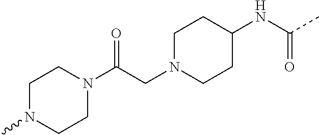 |
| 45 | 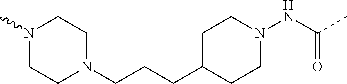 |
| 46 | 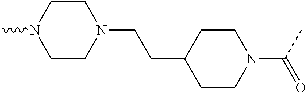 |
| 47 | 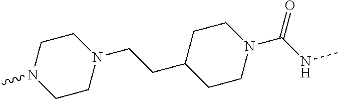 |
| 48 | 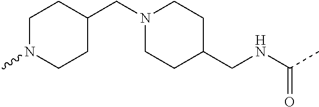 |
| 49 | 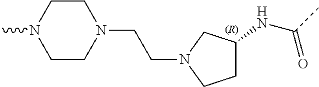 |
| 50 | 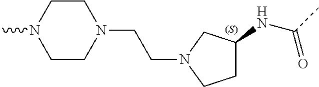 |
| 51 | 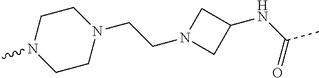 |
| 52 | 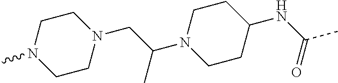 |
| 53 | 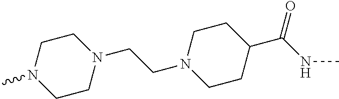 |
| 54 | 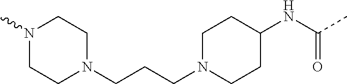 |
| 55 | 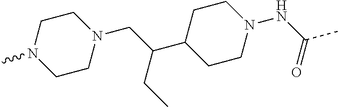 |
| 56 | 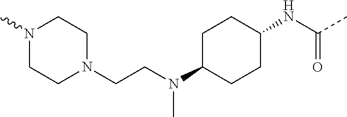 |
| 57 | 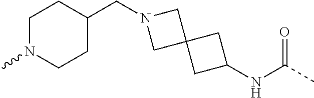 |
| 58 | 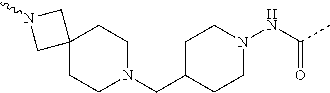 |
| 59 | 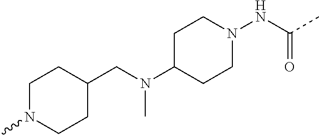 |
| 60 | 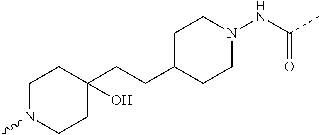 |
| 61 | 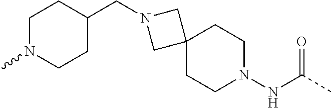 |
| 62 | 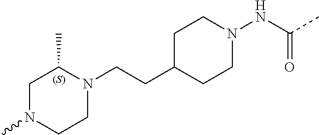 |
| 63 | 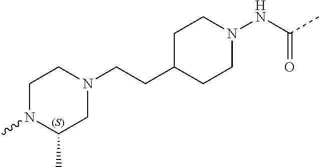 |
| 64 | 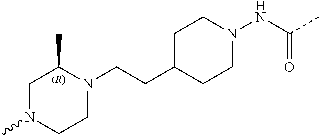 |
| 65 | 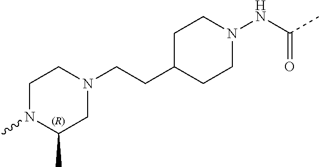 |
| 66 | 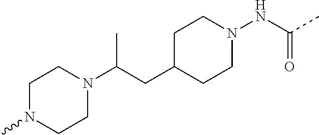 |
| 67 | 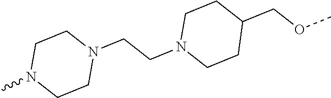 |
| 68 | 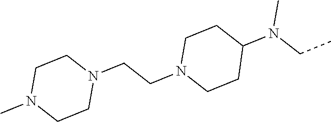 |

In one embodiment of the present disclosure, the compound represented by Formula I is selected from the group consisting of Compounds 1 to 123.

In one embodiment, the compound of the present disclosure may be in the form of a salt, preferably pharmaceutically acceptable salts. In the present disclosure, a pharmaceutically acceptable salt refers to any organic or inorganic acid addition salt with a concentration that is relatively non-toxic, is harmless, and has effective action to patients, wherein side effects caused by this salt does not deteriorate beneficial efficacy of the novel compounds of the present disclosures.

In one embodiment, the compound of the present disclosure may be in the form of a racemate, enantiomer, rotamer, tautomer, N-oxide, or any stereoisomer as if each is specifically described unless specifically excluded by context.

In one embodiment, the compound of the present disclosure may be in the form of a hydrate or solvate thereof,

In one embodiment, the compound of the present disclosure may be in the form of a chimeric molecule by conjugation to a functional macromolecule via a chemical linker. In certain embodiment, the macromolecule is a biomolecule including a nucleic acid, an aptamer, a carbohydrate, a peptide or fragment thereof, an antibody or fragment thereof.

In the present disclosure, “optionally substituted with” means a certain chemical group is unsubstituted or at least one hydrogen atom(s) in the group is replaced by a substituent.

In the present disclosure, the term “substituent” refers to a moiety of the parent molecule wherein at least one hydrogen atom(s) in the molecule is replaced to at least one covalent bond(s).

In the present disclosure, the term “alkyl” refers to a substituent of an alkane which is a saturated, acyclic hydrocarbon that may be linear or branched. For example, “Cₙ alkyl” means an alkyl that has n carbon atoms.

In the present disclosure, the term “cycloalkyl” refers to a substituent of a cycloalkane which is a saturated cyclic hydrocarbon.

In the present disclosure, the term “heterocyclyl” is a substituent of a heterocyclic compound consisting of at least one cyclic ring that has atoms of at least two different elements as members of its ring. For example, heterocyclyl may include carbon and nitrogen as members of its ring, such as azetidinyl, pyrrolidinyl, pyrazolidinyl, imidazolidinyl, piperidinyl, piperazinyl, hexahydropyridazinyl, hexahydropyrimidinyl, 1,2,3-triazinyl, 1,2,4-triazinyl, 1,3,5-triazinyl, and the like.

In the present disclosure, the term “aryl” is a substituent of a cyclic compound including at least one aromatic ring(s).

In the present disclosure, the term “heteroaryl” is a substituent of a cyclic compound including at least one aromatic ring(s) that have atoms of at least two different elements as members of its ring(s). For example, heteroaryl may include carbon and nitrogen as members of its ring, such as pyrrole, pyrazole, imidazole, triazole, triazine, tetrazole, pyridine, pyridazine, pyrimidine, pyrazine, indole, isoindole, indolizine, azaindolizine, purine, indazole, imidazopyridine, imidazotriazine, benzimidazole, pyrrolopyridine, pyrrolopyrimidine, pyridopyrimidine, quinoline, dihydroquinoline, tetrahydroquinoline, isoquinoline, dihydroisoquinoline, tetrahydroisoquinoline, quinolizine, phthalazine, naphthyridine, quinoxaline, quinazoline, cinnoline, pteridine, pyrazinopyridazine, and the like.

In the present disclosure, the term “n-membered” means the number of elements except hydrogen in the substituent is n.

In the present disclosure, “cycloalkyl”, “aryl”, “heterocyclyl” and “heteroaryl” above may be in the forms of monocyclic, fused cyclic (e.g., spiro or bicyclic) as well as polycyclic. For example, the cycloalkyl, aryl, heterocyclyl and “heteroaryl may be azaspiro, diazaspiro, bicyclo, azabicyclo, diazabicyclo, diazabicyclo(bridged) or tetrahydroisoquinoline, and the like.

### Synthesis of Novel AURKA Degraders

In one embodiment, the novel compound of the present disclosure may be prepared by for example, the following Reaction Schemes 1 to 3, by a synthetic method known in the field of organic chemistry or a modification technique apparent to those skilled in the art.

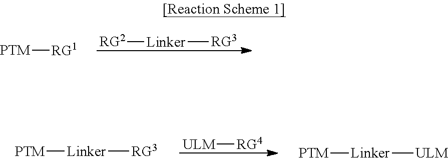

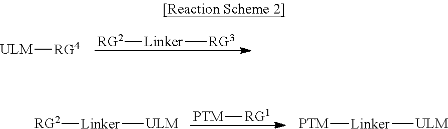

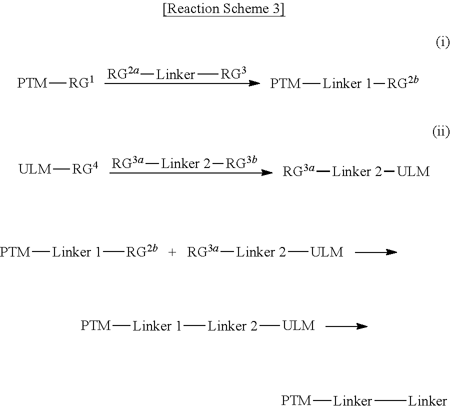

In the Reaction Schemes, PTM, Linker and ULM are same as defined above.

RG¹, RG², RG^{2a}, RG^{2b}, RG³, RG^{3a}, RG^{3b} and RG⁴ are moieties including a suitable reactive group capable of linking together with an intermediate of PROTACs through formation of the covalent bond in the field of organic synthesis. The formation of the covalent bond may be achieved by synthetic reactions such as amide formation, ester formation, carbamate formation, urea formation, ether formation, amine formation, and single bonds, double bond formation between various carbons, click chemistry and the like, depending on specific reaction groups, but is not limited thereto.

Each step in the above Reaction Scheme may include one or multiple synthesis steps. Isolation and purification of the product may be accomplished by standard procedures known to those skilled in the art of organic chemistry.

### Use of Novel AURKA Degraders

In one embodiment, the novel compound of the present disclosure is AURKA degrader that induces AURKA protein degradation in cells.

The PROTACs of the invention may induce proteasomal degradation of AURKA in cells, by recruiting CRBN ubiquitin ligase to AURKA that are supported by the experimental examples of the present disclosure. For example, the present disclosure reveals Compounds 1 to 123 have AURKA degradation activity evidenced by luciferase assay on HeLa LgBit cell system; leading to inhibitory activity on cancer cell viability evidenced by cell viability assay for small cell lung cancer (SCLC) cell lines NCI-H446.

The inventors revealed newly designed PROTACs with CRBN E3 ubiquitin ligase binding moieties as represented by the Formula III-1, III-2A or III-2B exhibits sufficient level of AURKA degradation efficiency throughout entire scope. In the fields of PROTACs, these are surprising results that are not expected from early stage PROTACs with thalidomide-based CRBN binders.

In one embodiment, the PROTACs of the invention has therapeutic efficacy better than small AURKA inhibitors from which the PROTACs originate, by depletion of target protein than inhibiting the same. Accordingly, the PROTACs of the invention may utilized for the treatment of AURKA-related disorder or condition, wherein abnormal expression of AURKA proteins involves in onset and/or progress of disease.

In the present disclosure, AURKA-related disorder or condition refer to any disease or condition capable of being treated, delayed, inhibited or prevented from induction of degradation or inhibition of activity of AURKA. AURKA-related disorder or condition include, but not limited thereto, a cancer, a benign tumor or a neurological disease, etc.

In one embodiment, the PROTACs of the inventions may have anticancer activity on AURKA expressing cancer cells by depletion of AURKA, a key oncogenic regulator of the cell cycle. The cancer includes all cancers capable of exhibiting prophylactic or therapeutic efficacy due to inhibition of AURKA activity, and may be solid cancer or blood cancer. For example, the cancer may be one or more selected from the group consisting of squamous cell carcinoma, small cell lung cancer, non-small cell lung cancer, lung adenocarcinoma, lung squamous cell carcinoma, peritoneal cancer, skin cancer, skin or intraocular melanoma, rectal cancer, anal muscle cancer, esophageal cancer, small intestine cancer, endocrine cancer, parathyroid cancer, adrenal cancer, soft tissue sarcoma, urethral cancer, chronic or acute leukemia, lymphocytic lymphoma, hepatocellular carcinoma, gastrointestinal cancer, gastric cancer, pancreatic cancer, glioblastoma, neuroblastoma, glioma, cervical cancer, ovarian cancer, liver cancer, bladder cancer, liver tumor, breast cancer, colon cancer, colorectal cancer, endometrial or uterine cancer, salivary gland cancer, kidney cancer, prostate cancer, vulvar cancer, thyroid cancer, head and neck cancer, brain cancer, osteosarcoma, bone cancer, large cell lymphoma, adrenocorticoid tumor, t cell lymphoma/leukemia, neuroendocrine cancer, neuroendocrine tumor, cholangiocarcinoma, and the like, but is not limited thereto. The cancer includes not only primary cancer but also metastatic cancer.

The benign tumors include all benign tumors capable of exhibiting prophylactic or therapeutic efficacy due to the inhibition of AURKA activity, such as benign tumors in pre-cancer stages, and may be solid tumors or blood tumors. For example, the tumor may be one or more selected from the group consisting of Barrett's esophagus, colon adenoma and polyp, breast fibroadenoma and cyst, monoclonal gammopathy of undetermined significance (MGUS), monoclonal lymphocytosis, and the like, but is not limited thereto.

The neurological diseases include all neurological diseases capable of exhibiting prophylactic or therapeutic efficacy due to the inhibition of AURKA activity, and specifically, may be one or more selected from the group consisting of central nervous system disease, neurodegenerative disease, Alzheimer's disease, Parkinson's disease, multiple sclerosis, Huntington's disease, senile dementia, epilepsy, Lou Gehrig, stroke, and nerve damage and axonal degeneration-related disorders following brain or spinal cord injury, but is not limited thereto.

To improve drug activities, any known strategies in the fields of PROTACs may be applied to the PROTACs of the present, including photochemically controllable PROTACs (PHOTACs), hypoxia-activated PROTACs, folate-caged PROTACs, antibody-PROTAC conjugates (Ab-PROTACs) and aptamer-PROTAC conjugates (APCs), and BCL-XL PROTACs, and the like (Zhao, Chunlong, and Frank J. Dekker. “Novel Design Strategies to Enhance the Efficiency of Proteolysis Targeting Chimeras.” ACS Pharmacology & Translational Science 5.9 (2022): 710-723.).

In one embodiment, the PROTACs of the invention may be utilized as a payload of antibody-drug conjugates (ADCs), for example, of antibody-PROTAC conjugates (Ab-PROTACs). ADCs enable the delivery of a cytotoxic payload specifically to cancer cells, enabling one to achieve a maximal effect on cancer cells whereas undesired effects in noncancer cells can be minimized. Therefore, Ab-PROTACs utilizing the PROTACs of the invention may be a strategy to improve the tissue and cell-type selectivity of PROTACs.

In one embodiment, the present disclosure provides a pharmaceutical composition comprising the PROTACs of the invention, wherein the compound is conjugated to an antibody or an antigen binding fragment thereof via a linker.

In one embodiment, the present disclosure provides an antibody-drug conjugate comprising an antibody or an antigen binding fragment thereof and the PROTACs of the invention, wherein the compound is conjugated to the antibody or antigen binding fragment thereof via a linker.

In certain embodiment, the linker chemically connects the antibody or an antigen binding fragment thereof and the PROTACs of the invention wherein a functional group within the compound is modified to create a covalent bond with the linker moiety. In certain embodiment, the functional group is an amine group within PROTAC linker or E3L binder moiety of the compounds.

In certain embodiment, the antibody or antigen binding fragment thereof is a cancer cell-specific and bearing one or more molecules of the compounds of Formula I. In certain embodiment, the linker is a cleavable or non-cleavable linker.

The present disclosure also provides a pharmaceutical composition comprising the PROTACs of the invention, and at least one pharmaceutically acceptable carrier. In one embodiment, the pharmaceutical composition comprises an effective amount of at least one PROTAC compound of the invention, and optionally one or more of other active ingredient(s) in effective amounts for combination therapy. In one embodiment, the pharmaceutical composition comprises more than one pharmaceutically acceptable amount of additive or excipient.

In another embodiment of the present disclosure is a method of degrading AURKA by administering the PROTACs of the invention to a sample in vitro. The sample may include a cell, a cell culture, a body fluid or tissue of a mammal including a human, but is not limited thereto.

### Advantageous Effects of Invention

The novel compounds of the present disclosure may induce AURKA degradation in cells throughout entire scope; therefore, they may be effectively utilized for treatment of AURKA-related disorder or conditions.

### BRIEF DESCRIPTION OF DRAWINGS

FIG. 1 depicts the principle of PROTAC-driven target protein degradation, wherein recruitment of E3 ubiquitin ligase to the target protein leads to ubiquitination and proteasomal degradation of the target protein AURKA.

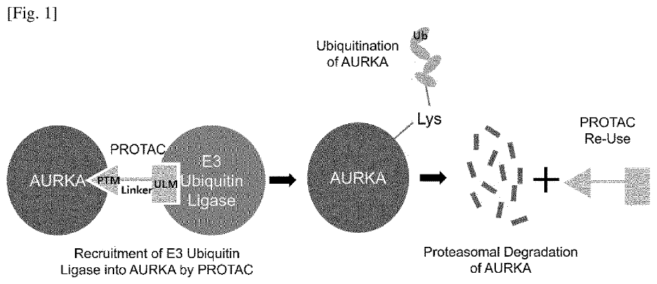

### BEST MODE FOR CARRYING OUT THE INVENTION

Unless otherwise defined, all technical and scientific terms used herein have the same meaning as commonly understood by one of ordinary skill in the art to which this disclosure belongs. The terminology used in the description is for describing particular embodiments only and is not intended to be limiting of the disclosure.

The present disclosure provides synthetic methods and results of bioactivity of Compounds 1 to 123.

TABLE 5

| No. | Structure |
|---|---|
| 1 | 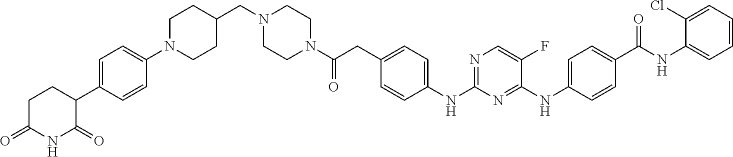 [SMILES: O=C1CCC(c2ccc(N3CCC(CN4CCN(C(=O)Cc5ccc(Nc6ncc(F)c(Nc7ccc(C(=O)Nc8ccccc8Cl)cc7)n6)cc5)CC4)CC3)cc2)C(=O)N1] |
| 2 | 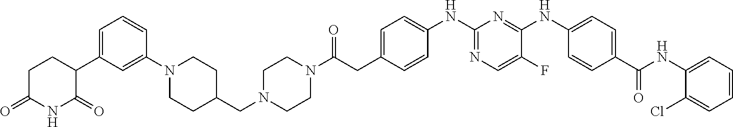 [SMILES: O=C1CCC(c2cccc(N3CCC(CN4CCN(C(=O)Cc5ccc(Nc6ncc(F)c(Nc7ccc(C(=O)Nc8ccccc8Cl)cc7)n6)cc5)CC4)CC3)c2)C(=O)N1] |
| 3 | 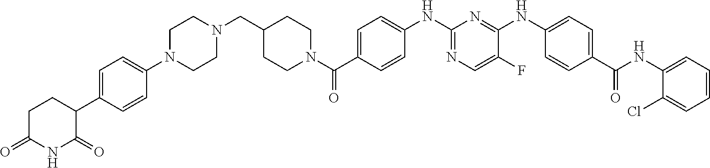 [SMILES: O=C1CCC(c2ccc(N3CCN(CC4CCN(C(=O)c5ccc(Nc6ncc(F)c(Nc7ccc(C(=O)Nc8ccccc8Cl)cc7)n6)cc5)CC4)CC3)cc2)C(=O)N1] |
| 4 | 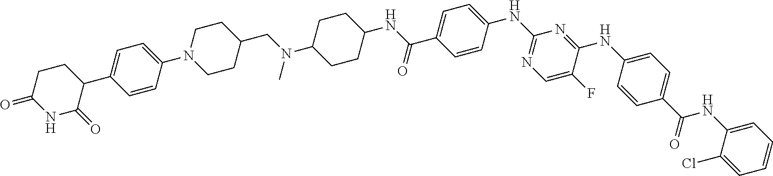 [SMILES: CN(CC1CCN(c2ccc(C3CCC(=O)NC3=O)cc2)CC1)C1CCC(NC(=O)c2ccc(Nc3ncc(F)c(Nc4ccc(C(=O)Nc5ccccc5Cl)cc4)n3)cc2)CC1] |
| 5 | 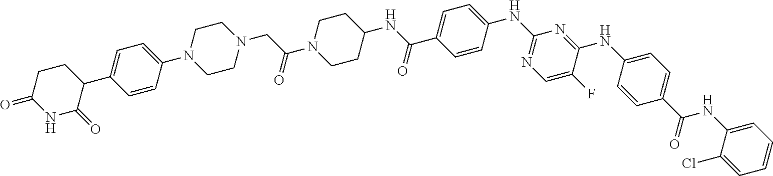 [SMILES: O=C1CCC(c2ccc(N3CCN(CC(=O)N4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 6 | 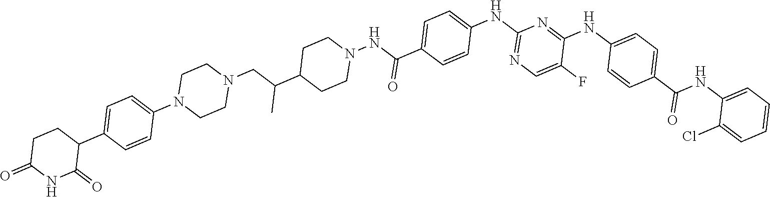 [SMILES: CC(CN1CCN(c2ccc(C3CCC(=O)NC3=O)cc2)CC1)C1CCN(NC(=O)c2ccc(Nc3ncc(F)c(Nc4ccc(C(=O)Nc5ccccc5Cl)cc4)n3)cc2)CC1] |
| 7 | 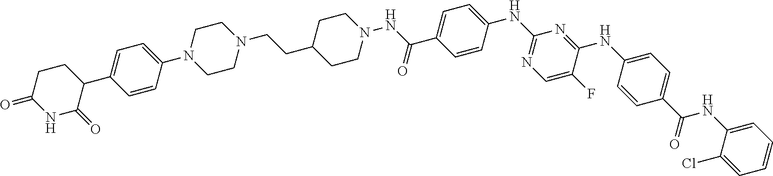 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2)C(=O)N1] |
| 8 | 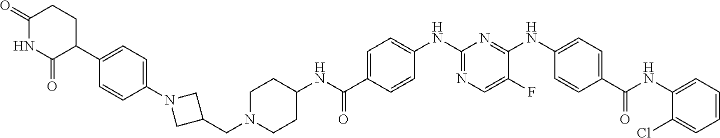 [SMILES: O=C1CCC(c2ccc(N3CC(CN4CCC(NC(=O)c5ccc(Nc6ncc(F)c(Nc7ccc(C(=O)Nc8ccccc8Cl)cc7)n6)cc5)CC4)C3)cc2)C(=O)N1] |
| 9 | 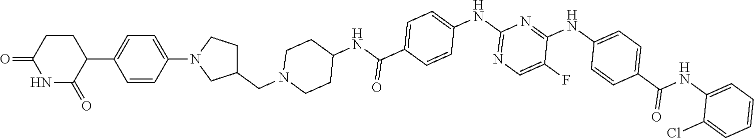 [SMILES: O=C1CCC(c2ccc(N3CCC(CN4CCC(NC(=O)c5ccc(Nc6ncc(F)c(Nc7ccc(C(=O)Nc8ccccc8Cl)cc7)n6)cc5)CC4)C3)cc2)C(=O)N1] |
| 10 | 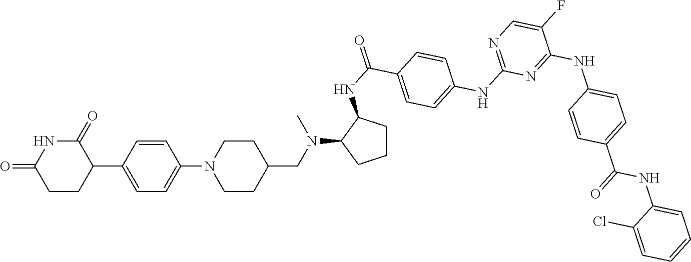 [SMILES: CN(CC1CCN(c2ccc(C3CCC(=O)NC3=O)cc2)CC1)[C@@H]1CCC[C@@H]1NC(=O)c1ccc(Nc2ncc(F)c(Nc3ccc(C(=O)Nc4ccccc4Cl)cc3)n2)cc1] |
| 11 | 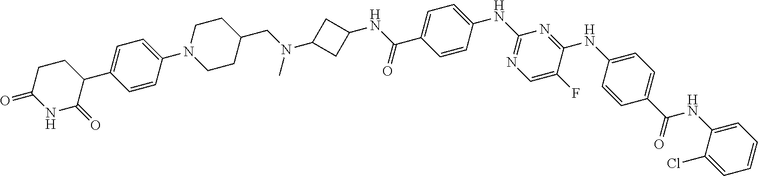 [SMILES: CN(CC1CCN(c2ccc(C3CCC(=O)NC3=O)cc2)CC1)C1CC(NC(=O)c2ccc(Nc3ncc(F)c(Nc4ccc(C(=O)Nc5ccccc5Cl)cc4)n3)cc2)C1] |
| 12 | 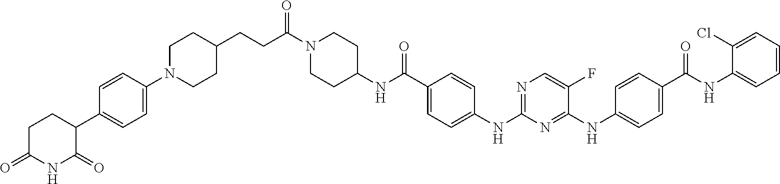 [SMILES: O=C1CCC(c2ccc(N3CCC(CCC(=O)N4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 13 | 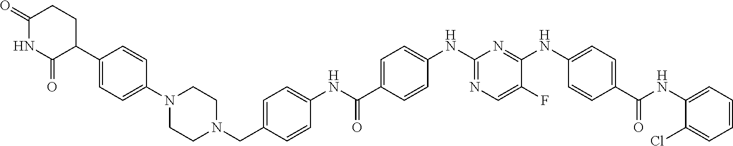 [SMILES: O=C1CCC(c2ccc(N3CCN(Cc4ccc(NC(=O)c5ccc(Nc6ncc(F)c(Nc7ccc(C(=O)Nc8ccccc8Cl)cc7)n6)cc5)cc4)CC3)cc2)C(=O)N1] |
| 14 | 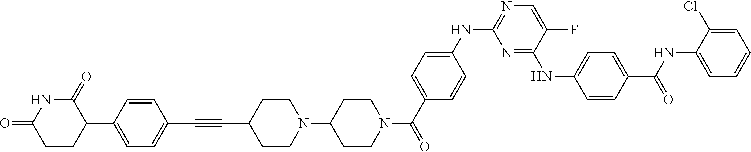 [SMILES: O=C1CCC(c2ccc(C#CC3CCN(C4CCN(C(=O)c5ccc(Nc6ncc(F)c(Nc7ccc(C(=O)Nc8ccccc8Cl)cc7)n6)cc5)CC4)CC3)cc2)C(=O)N1] |
| 15 | 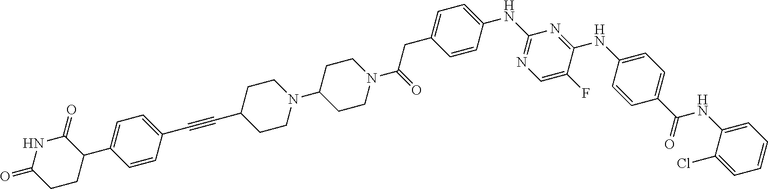 [SMILES: O=C1CCC(c2ccc(C#CC3CCN(C4CCN(C(=O)Cc5ccc(Nc6ncc(F)c(Nc7ccc(C(=O)Nc8ccccc8Cl)cc7)n6)cc5)CC4)CC3)cc2)C(=O)N1] |
| 16 | 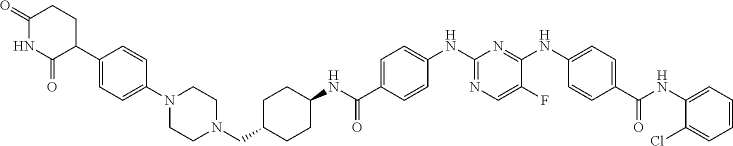 [SMILES: O=C1CCC(c2ccc(N3CCN(C[C@H]4CC[C@H](NC(=O)c5ccc(Nc6ncc(F)c(Nc7ccc(C(=O)Nc8ccccc8Cl)cc7)n6)cc5)CC4)CC3)cc2)C(=O)N1] |
| 17 | 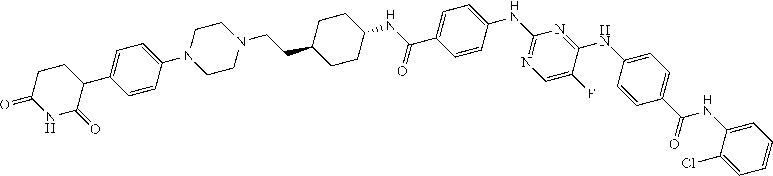 [SMILES: O=C1CCC(c2ccc(N3CCN(CC[C@H]4CC[C@H](NC(=O)c5ccc(Nc6ncc(F)c(Nc7ccc(C(=O)Nc8ccccc8Cl)cc7)n6)cc5)CC4)CC3)cc2)C(=O)N1] |
| 18 | 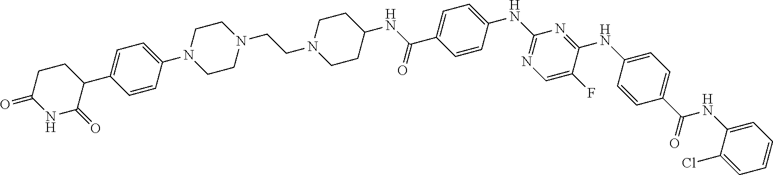 [SMILES: O=C1CCC(c2ccc(N3CCN(CCN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 19 | 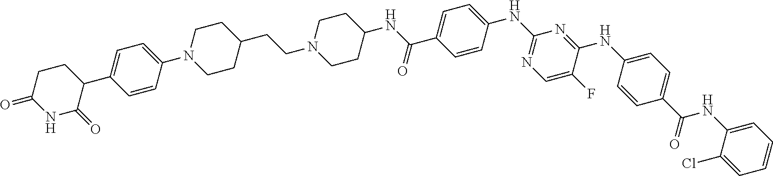 [SMILES: O=C1CCC(c2ccc(N3CCC(CCN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 20 | 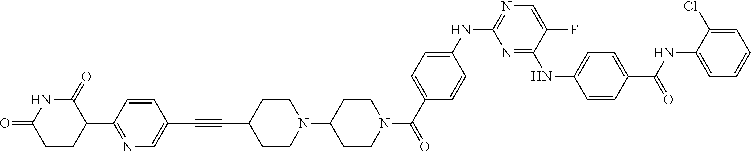 [SMILES: O=C1CCC(c2ccc(C#CC3CCN(C4CCN(C(=O)c5ccc(Nc6ncc(F)c(Nc7ccc(C(=O)Nc8ccccc8Cl)cc7)n6)cc5)CC4)CC3)cn2)C(=O)N1] |
| 21 | 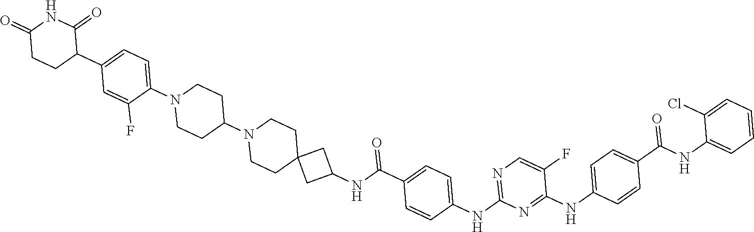 [SMILES: O=C1CCC(c2ccc(N3CCC(N4CCC5(CC4)CC(NC(=O)c4ccc(Nc6ncc(F)c(Nc7ccc(C(=O)Nc8ccccc8Cl)cc7)n6)cc4)C5)CC3)c(F)c2)C(=O)N1] |
| 22 | 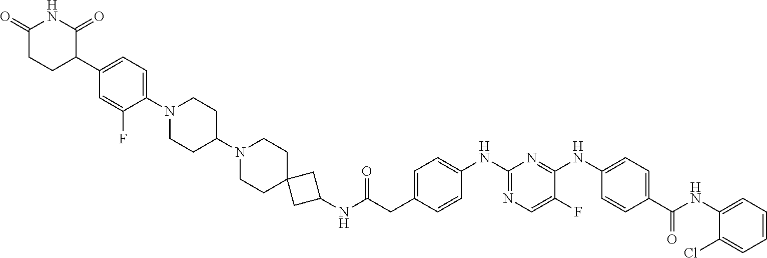 [SMILES: O=C1CCC(c2ccc(N3CCC(N4CCC5(CC4)CC(NC(=O)Cc4ccc(Nc6ncc(F)c(Nc7ccc(C(=O)Nc8ccccc8Cl)cc7)n6)cc4)C5)CC3)c(F)c2)C(=O)N1] |
| 23 | 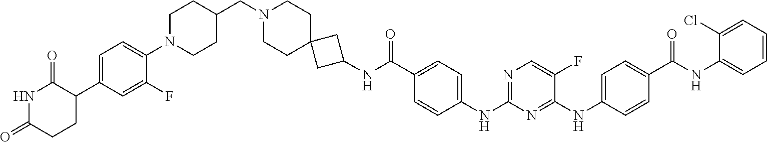 [SMILES: O=C1CCC(c2ccc(N3CCC(CN4CCC5(CC4)CC(NC(=O)c4ccc(Nc6ncc(F)c(Nc7ccc(C(=O)Nc8ccccc8Cl)cc7)n6)cc4)C5)CC3)c(F)c2)C(=O)N1] |
| 24 | 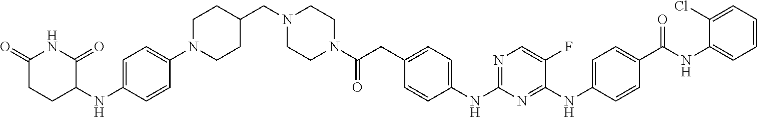 [SMILES: O=C1CCC(Nc2ccc(N3CCC(CN4CCN(C(=O)Cc5ccc(Nc6ncc(F)c(Nc7ccc(C(=O)Nc8ccccc8Cl)cc7)n6)cc5)CC4)CC3)cc2)C(=O)N1] |
| 25 | 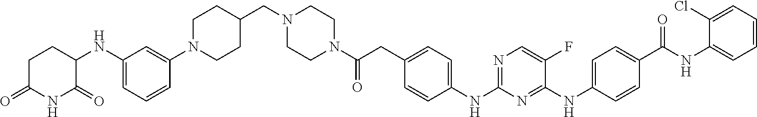 [SMILES: O=C1CCC(Nc2cccc(N3CCC(CN4CCN(C(=O)Cc5ccc(Nc6ncc(F)c(Nc7ccc(C(=O)Nc8ccccc8Cl)cc7)n6)cc5)CC4)CC3)c2)C(=O)N1] |
| 26 | 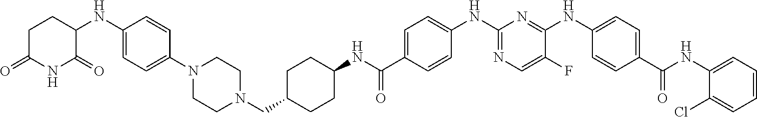 [SMILES: O=C1CCC(Nc2ccc(N3CCN(C[C@H]4CC[C@H](NC(=O)c5ccc(Nc6ncc(F)c(Nc7ccc(C(=O)Nc8ccccc8Cl)cc7)n6)cc5)CC4)CC3)cc2)C(=O)N1] |
| 27 | 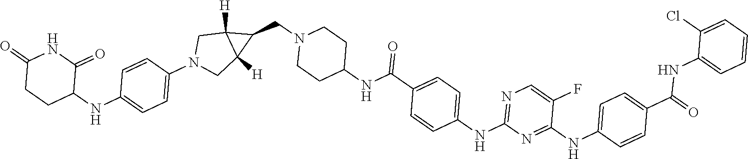 [SMILES: O=C1CCC(Nc2ccc(N3C[C@@H]4[C@@H](CN5CCC(NC(=O)c6ccc(Nc7ncc(F)c(Nc8ccc(C(=O)Nc9ccccc9Cl)cc8)n7)cc6)CC5)[C@@H]4C3)cc2)C(=O)N1] |
| 28 | 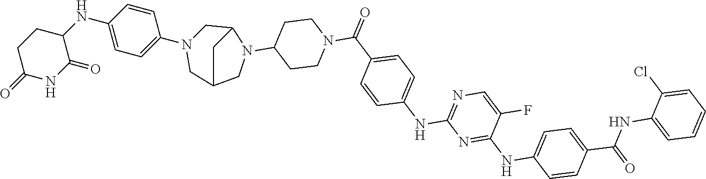 [SMILES: O=C1CCC(Nc2ccc(N3CC4CC(C3)N(C3CCN(C(=O)c5ccc(Nc6ncc(F)c(Nc7ccc(C(=O)Nc8ccccc8Cl)cc7)n6)cc5)CC3)C4)cc2)C(=O)N1] |
| 29 | 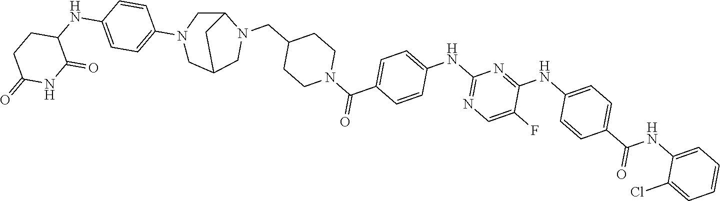 [SMILES: O=C1CCC(Nc2ccc(N3CC4CC(C3)N(CC3CCN(C(=O)c5ccc(Nc6ncc(F)c(Nc7ccc(C(=O)Nc8ccccc8Cl)cc7)n6)cc5)CC3)C4)cc2)C(=O)N1] |
| 30 | 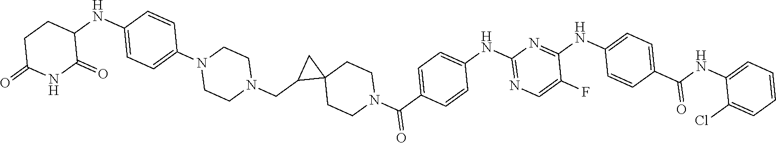 [SMILES: O=C1CCC(Nc2ccc(N3CCN(CC4CC45CCN(C(=O)c4ccc(Nc6ncc(F)c(Nc7ccc(C(=O)Nc8ccccc8Cl)cc7)n6)cc4)CC5)CC3)cc2)C(=O)N1] |
| 31 | 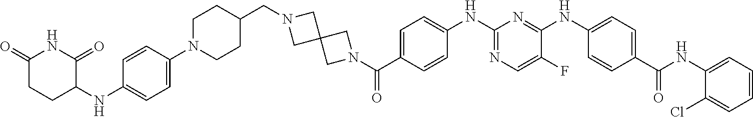 [SMILES: O=C1CCC(Nc2ccc(N3CCC(CN4CC5(C4)CN(C(=O)c4ccc(Nc6ncc(F)c(Nc7ccc(C(=O)Nc8ccccc8Cl)cc7)n6)cc4)C5)CC3)cc2)C(=O)N1] |
| 32 | 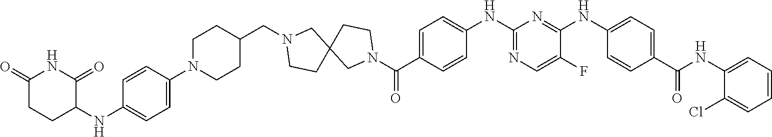 [SMILES: O=C1CCC(Nc2ccc(N3CCC(CN4CCC5(CCN(C(=O)c6ccc(Nc7ncc(F)c(Nc8ccc(C(=O)Nc9ccccc9Cl)cc8)n7)cc6)C5)C4)CC3)cc2)C(=O)N1] |
| 33 | 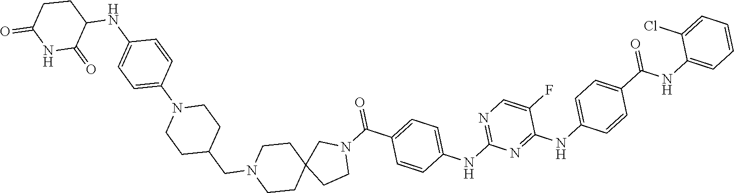 [SMILES: O=C1CCC(Nc2ccc(N3CCC(CN4CCC5(CC4)CCN(C(=O)c4ccc(Nc6ncc(F)c(Nc7ccc(C(=O)Nc8ccccc8Cl)cc7)n6)cc4)C5)CC3)cc2)C(=O)N1] |
| 34 | 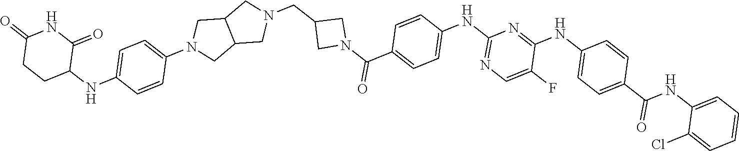 [SMILES: O=C1CCC(Nc2ccc(N3CC4CN(CC5CN(C(=O)c6ccc(Nc7ncc(F)c(Nc8ccc(C(=O)Nc9ccccc9Cl)cc8)n7)cc6)C5)CC4C3)cc2)C(=O)N1] |
| 35 | 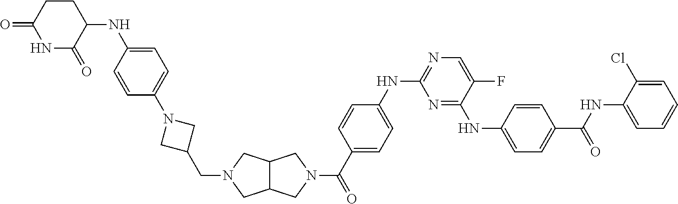 [SMILES: O=C1CCC(Nc2ccc(N3CC(CN4CC5CN(C(=O)c6ccc(Nc7ncc(F)c(Nc8ccc(C(=O)Nc9ccccc9Cl)cc8)n7)cc6)CC5C4)C3)cc2)C(=O)N1] |
| 36 | 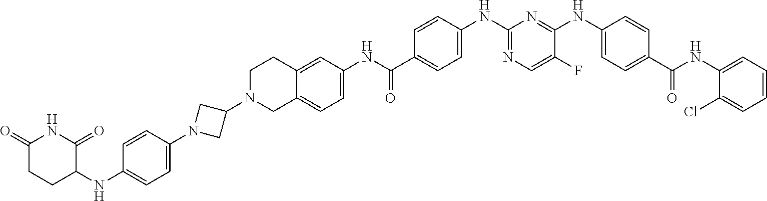 [SMILES: O=C1CCC(Nc2ccc(N3CC(N4CCc5cc(NC(=O)c6ccc(Nc7ncc(F)c(Nc8ccc(C(=O)Nc9ccccc9Cl)cc8)n7)cc6)ccc5C4)C3)cc2)C(=O)N1] |
| 37 | 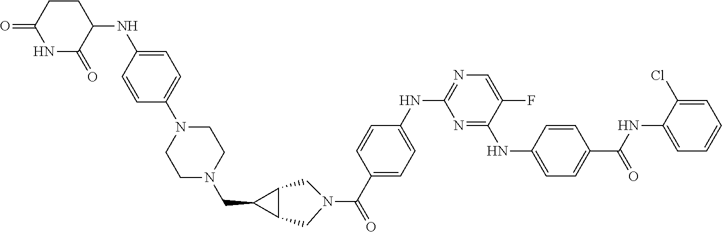 [SMILES: O=C1CCC(Nc2ccc(N3CCN(C[C@@H]4[C@H]5CN(C(=O)c6ccc(Nc7ncc(F)c(Nc8ccc(C(=O)Nc9ccccc9Cl)cc8)n7)cc6)C[C@@H]45)CC3)cc2)C(=O)N1] |
| 38 | 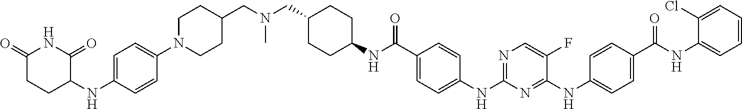 [SMILES: CN(CC1CCN(c2ccc(NC3CCC(=O)NC3=O)cc2)CC1)C[C@H]1CC[C@H](NC(=O)c2ccc(Nc3ncc(F)c(Nc4ccc(C(=O)Nc5ccccc5Cl)cc4)n3)cc2)CC1] |
| 39 | 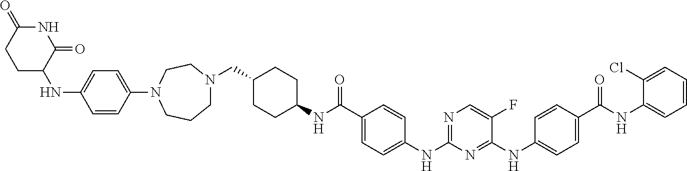 [SMILES: O=C1CCC(Nc2ccc(N3CCCN(C[C@H]4CC[C@H](NC(=O)c5ccc(Nc6ncc(F)c(Nc7ccc(C(=O)Nc8ccccc8Cl)cc7)n6)cc5)CC4)CC3)cc2)C(=O)N1] |
| 40 | 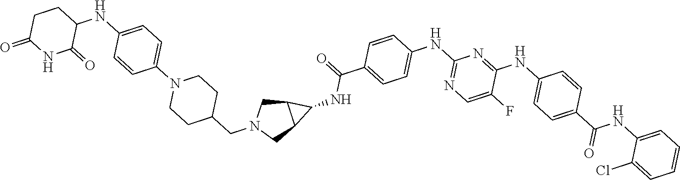 [SMILES: O=C1CCC(Nc2ccc(N3CCC(CN4C[C@@H]5[C@H](C4)[C@H]5NC(=O)c4ccc(Nc5ncc(F)c(Nc6ccc(C(=O)Nc7ccccc7Cl)cc6)n5)cc4)CC3)cc2)C(=O)N1] |
| 41 | 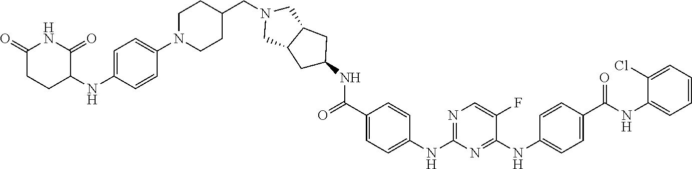 [SMILES: O=C1CCC(Nc2ccc(N3CCC(CN4C[C@H]5C[C@H](NC(=O)c6ccc(Nc7ncc(F)c(Nc8ccc(C(=O)Nc9ccccc9Cl)cc8)n7)cc6)C[C@H]5C4)CC3)cc2)C(=O)N1] |
| 42 | 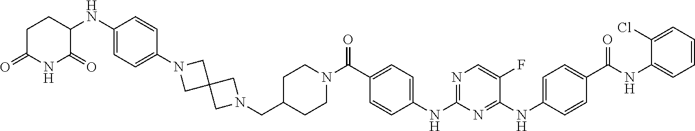 [SMILES: O=C1CCC(Nc2ccc(N3CC4(CN(CC5CCN(C(=O)c6ccc(Nc7ncc(F)c(Nc8ccc(C(=O)Nc9ccccc9Cl)cc8)n7)cc6)CC5)C4)C3)cc2)C(=O)N1] |
| 43 | 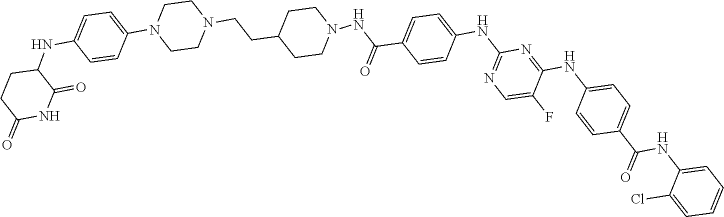 [SMILES: O=C1CCC(Nc2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2)C(=O)N1] |
| 44 | 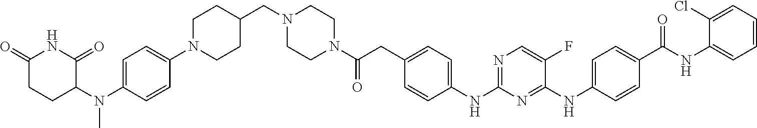 [SMILES: CN(c1ccc(N2CCC(CN3CCN(C(=O)Cc4ccc(Nc5ncc(F)c(Nc6ccc(C(=O)Nc7ccccc7Cl)cc6)n5)cc4)CC3)CC2)cc1)C1CCC(=O)NC1=O] |
| 45 | 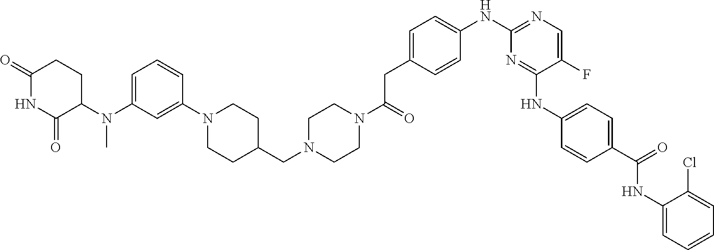 [SMILES: CN(c1cccc(N2CCC(CN3CCN(C(=O)Cc4ccc(Nc5ncc(F)c(Nc6ccc(C(=O)Nc7ccccc7Cl)cc6)n5)cc4)CC3)CC2)c1)C1CCC(=O)NC1=O] |
| 46 | 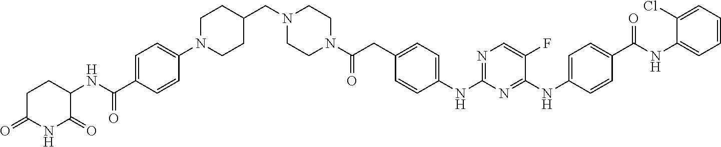 [SMILES: O=C1CCC(NC(=O)c2ccc(N3CCC(CN4CCN(C(=O)Cc5ccc(Nc6ncc(F)c(Nc7ccc(C(=O)Nc8ccccc8Cl)cc7)n6)cc5)CC4)CC3)cc2)C(=O)N1] |
| 47 | 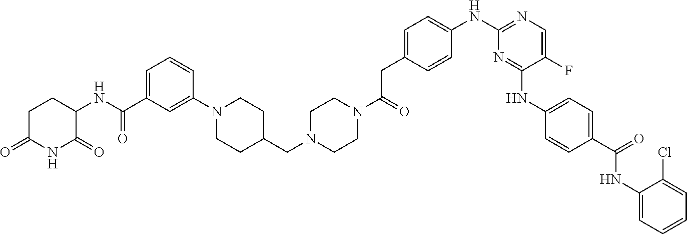 [SMILES: O=C1CCC(NC(=O)c2cccc(N3CCC(CN4CCN(C(=O)Cc5ccc(Nc6ncc(F)c(Nc7ccc(C(=O)Nc8ccccc8Cl)cc7)n6)cc5)CC4)CC3)c2)C(=O)N1] |
| 48 | 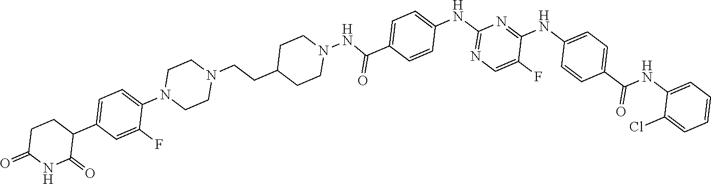 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)c(F)c2)C(=O)N1] |
| 49 | 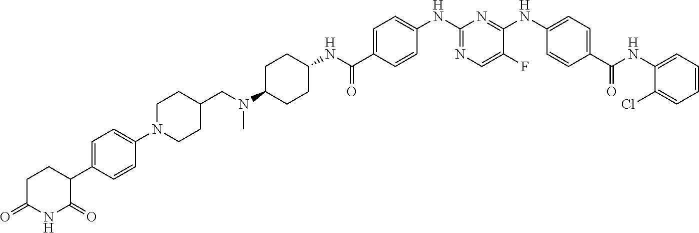 [SMILES: CN(CC1CCN(c2ccc(C3CCC(=O)NC3=O)cc2)CC1)[C@H]1CC[C@H](NC(=O)c2ccc(Nc3ncc(F)c(Nc4ccc(C(=O)Nc5ccccc5Cl)cc4)n3)cc2)CC1] |
| 50 | 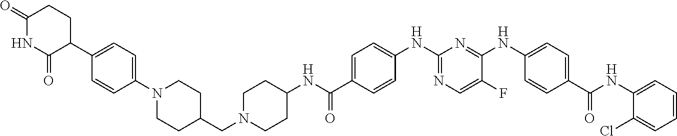 [SMILES: O=C1CCC(c2ccc(N3CCC(CN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 51 | 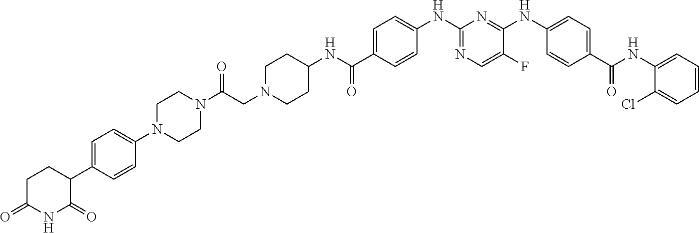 [SMILES: O=C1CCC(c2ccc(N3CCN(C(=O)CN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 52 | 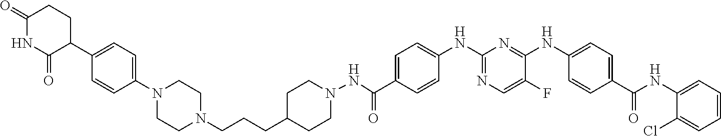 [SMILES: O=C1CCC(c2ccc(N3CCN(CCCC4CCN(NC(=O)c5ccc(Nc6ncc(F)c(Nc7ccc(C(=O)Nc8ccccc8Cl)cc7)n6)cc5)CC4)CC3)cc2)C(=O)N1] |
| 53 | 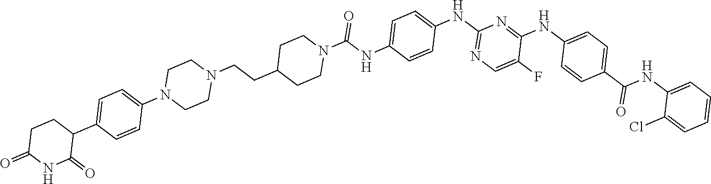 [SMILES: O=C1CCC(c2ccc(N3CCN(CCC4CCN(C(=O)Nc5ccc(Nc6ncc(F)c(Nc7ccc(C(=O)Nc8ccccc8Cl)cc7)n6)cc5)CC4)CC3)cc2)C(=O)N1] |
| 54 | 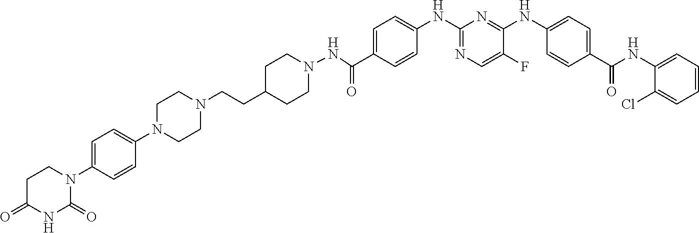 [SMILES: O=C1CCN(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2)C(=O)N1] |
| 55 | 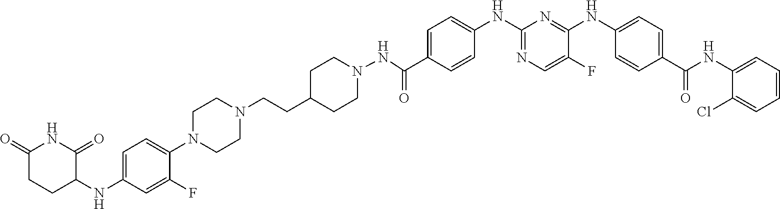 [SMILES: O=C1CCC(Nc2ccc(N3CCN(CCC4CCN(NC(=O)c5ccc(Nc6ncc(F)c(Nc7ccc(C(=O)Nc8ccccc8Cl)cc7)n6)cc5)CC4)CC3)c(F)c2)C(=O)N1] |
| 56 | 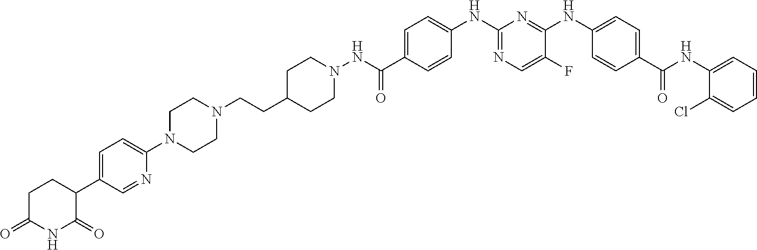 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)nc2)C(=O)N1] |
| 57 | 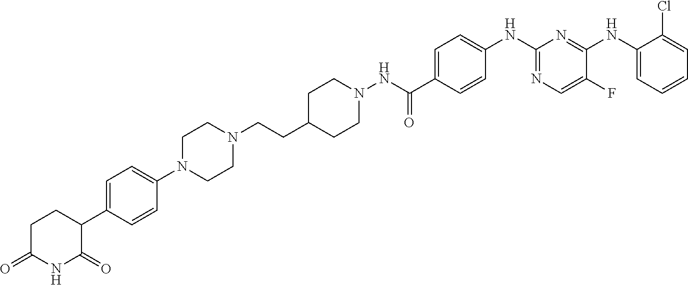 [SMILES: O=C1CCC(c2ccc(N3CCN(CCC4CCN(NC(=O)c5ccc(Nc6ncc(F)c(Nc7ccccc7Cl)n6)cc5)CC4)CC3)cc2)C(=O)N1] |
| 58 | 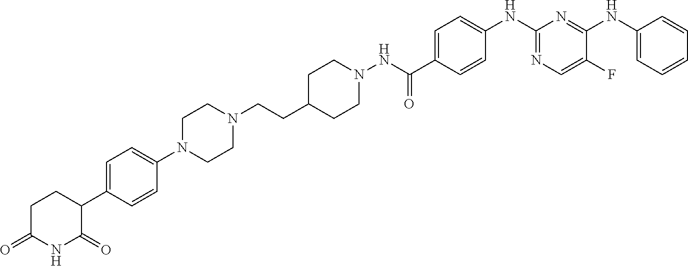 [SMILES: O=C1CCC(c2ccc(N3CCN(CCC4CCN(NC(=O)c5ccc(Nc6ncc(F)c(Nc7ccccc7)n6)cc5)CC4)CC3)cc2)C(=O)N1] |
| 59 | 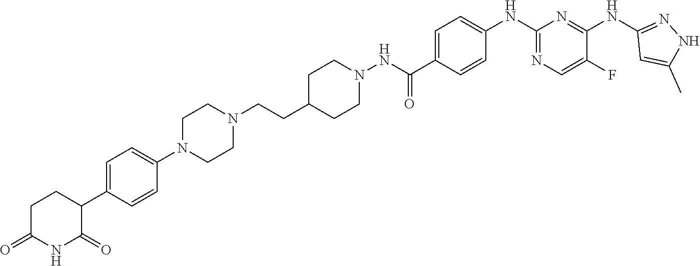 [SMILES: Cc1cc(Nc2nc(Nc3ccc(C(=O)NN4CCC(CCN5CCN(c6ccc(C7CCC(=O)NC7=O)cc6)CC5)CC4)cc3)ncc2F)n[nH]1] |
| 60 | 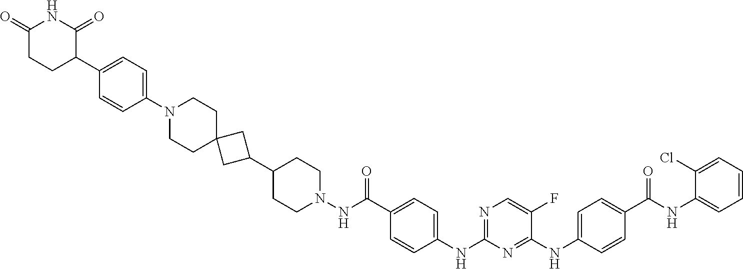 [SMILES: O=C1CCC(c2ccc(N3CCC4(CC3)CC(C3CCN(NC(=O)c5ccc(Nc6ncc(F)c(Nc7ccc(C(=O)Nc8ccccc8Cl)cc7)n6)cc5)CC3)C4)cc2)C(=O)N1] |
| 61 | 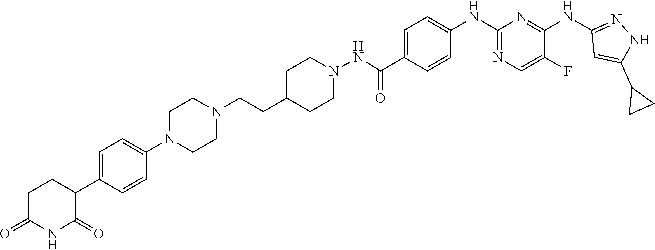 [SMILES: O=C1CCC(c2ccc(N3CCN(CCC4CCN(NC(=O)c5ccc(Nc6ncc(F)c(Nc7cc(C8CC8)[nH]n7)n6)cc5)CC4)CC3)cc2)C(=O)N1] |
| 62 | 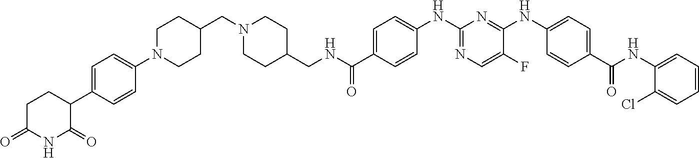 [SMILES: O=C1CCC(c2ccc(N3CCC(CN4CCC(CNC(=O)c5ccc(Nc6ncc(F)c(Nc7ccc(C(=O)Nc8ccccc8Cl)cc7)n6)cc5)CC4)CC3)cc2)C(=O)N1] |
| 63 | 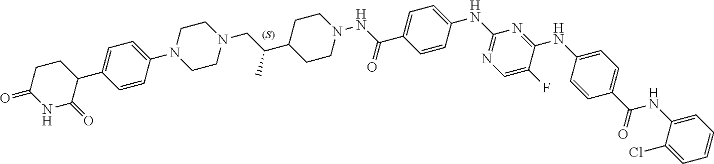 |
| 64 | 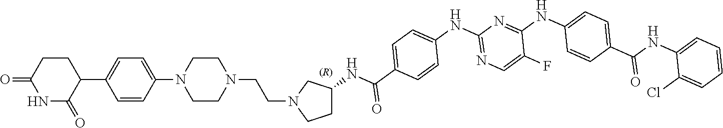 |
| 65 | 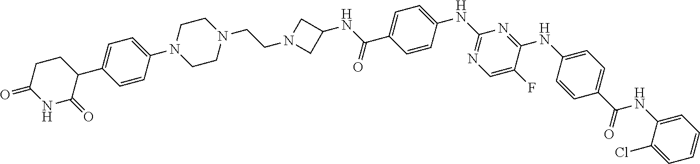 [SMILES: O=C1CCC(c2ccc(N3CCN(CCN4CC(NC(=O)c5ccc(Nc6ncc(F)c(Nc7ccc(C(=O)Nc8ccccc8Cl)cc7)n6)cc5)C4)CC3)cc2)C(=O)N1] |
| 66 | 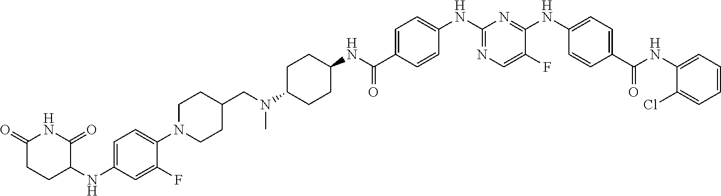 [SMILES: CN(CC1CCN(c2ccc(NC3CCC(=O)NC3=O)cc2F)CC1)[C@H]1CC[C@H](NC(=O)c2ccc(Nc3ncc(F)c(Nc4ccc(C(=O)Nc5ccccc5Cl)cc4)n3)cc2)CC1] |
| 67 | 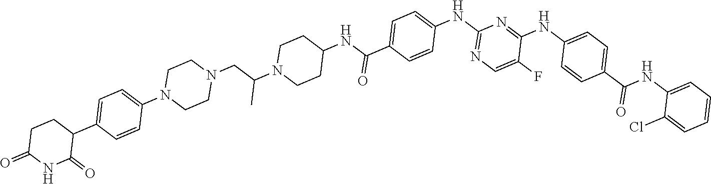 [SMILES: CC(CN1CCN(c2ccc(C3CCC(=O)NC3=O)cc2)CC1)N1CCC(NC(=O)c2ccc(Nc3ncc(F)c(Nc4ccc(C(=O)Nc5ccccc5Cl)cc4)n3)cc2)CC1] |
| 68 | 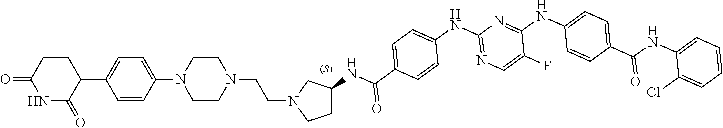 |
| 69 | 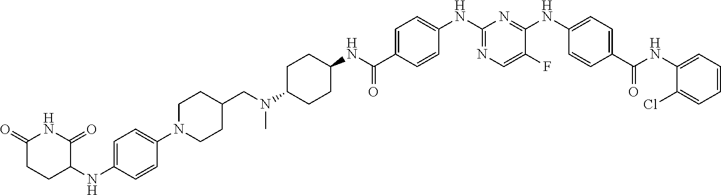 [SMILES: CN(CC1CCN(c2ccc(NC3CCC(=O)NC3=O)cc2)CC1)[C@H]1CC[C@H](NC(=O)c2ccc(Nc3ncc(F)c(Nc4ccc(C(=O)Nc5ccccc5Cl)cc4)n3)cc2)CC1] |
| 70 | 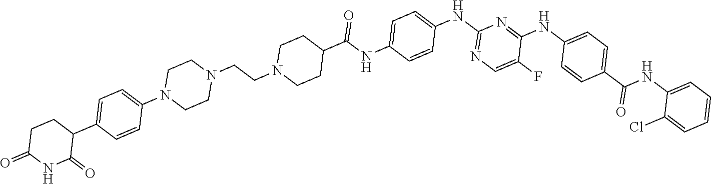 [SMILES: O=C1CCC(c2ccc(N3CCN(CCN4CCC(C(=O)Nc5ccc(Nc6ncc(F)c(Nc7ccc(C(=O)Nc8ccccc8Cl)cc7)n6)cc5)CC4)CC3)cc2)C(=O)N1] |
| 71 | 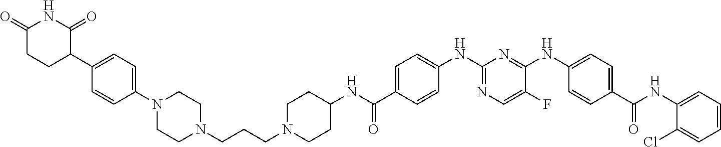 [SMILES: O=C1CCC(c2ccc(N3CCN(CCCN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 72 | 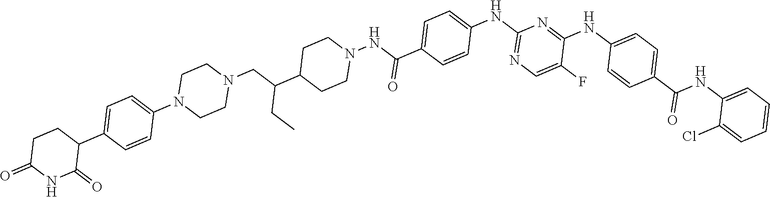 [SMILES: CCC(CN1CCN(c2ccc(C3CCC(=O)NC3=O)cc2)CC1)C1CCN(NC(=O)c2ccc(Nc3ncc(F)c(Nc4ccc(C(=O)Nc5ccccc5Cl)cc4)n3)cc2)CC1] |
| 73 | 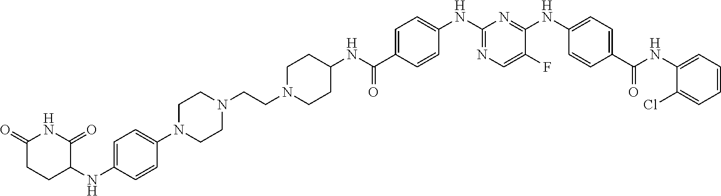 [SMILES: O=C1CCC(Nc2ccc(N3CCN(CCN4CCC(NC(=O)c5ccc(Nc6ncc(F)c(Nc7ccc(C(=O)Nc8ccccc8Cl)cc7)n6)cc5)CC4)CC3)cc2)C(=O)N1] |
| 74 | 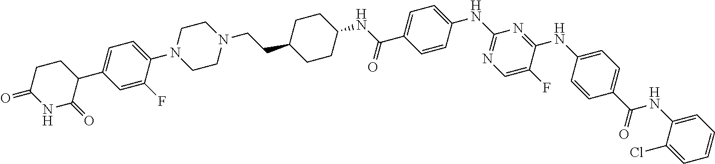 [SMILES: O=C1CCC(c2ccc(N3CCN(CC[C@H]4CC[C@H](NC(=O)c5ccc(Nc6ncc(F)c(Nc7ccc(C(=O)Nc8ccccc8Cl)cc7)n6)cc5)CC4)CC3)c(F)c2)C(=O)N1] |
| 75 | 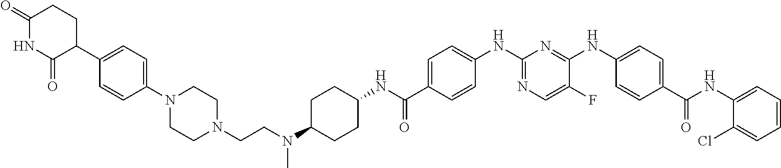 [SMILES: CN(CCN1CCN(c2ccc(C3CCC(=O)NC3=O)cc2)CC1)[C@H]1CC[C@H](NC(=O)c2ccc(Nc3ncc(F)c(Nc4ccc(C(=O)Nc5ccccc5Cl)cc4)n3)cc2)CC1] |
| 76 | 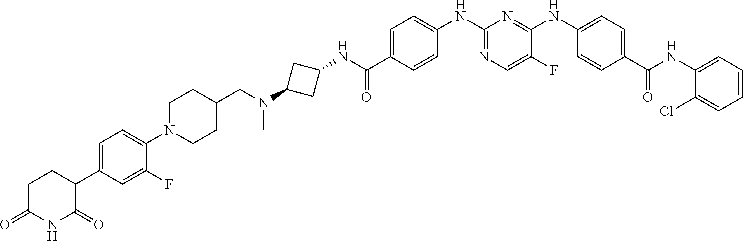 [SMILES: CN(CC1CCN(c2ccc(C3CCC(=O)NC3=O)cc2F)CC1)[C@H]1C[C@H](NC(=O)c2ccc(Nc3ncc(F)c(Nc4ccc(C(=O)Nc5ccccc5Cl)cc4)n3)cc2)C1] |
| 77 | 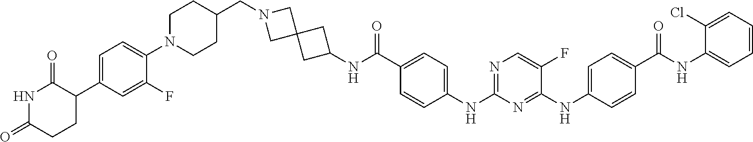 [SMILES: O=C1CCC(c2ccc(N3CCC(CN4CC5(CC(NC(=O)c6ccc(Nc7ncc(F)c(Nc8ccc(C(=O)Nc9ccccc9Cl)cc8)n7)cc6)C5)C4)CC3)c(F)c2)C(=O)N1] |
| 78 | 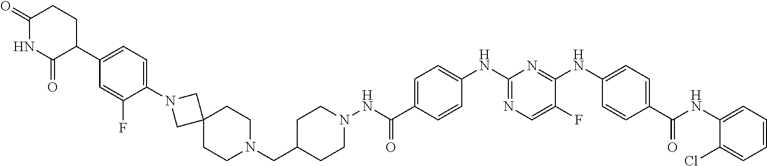 [SMILES: O=C1CCC(c2ccc(N3CC4(CCN(CC5CCN(NC(=O)c6ccc(Nc7ncc(F)c(Nc8ccc(C(=O)Nc9ccccc9Cl)cc8)n7)cc6)CC5)CC4)C3)c(F)c2)C(=O)N1] |
| 79 | 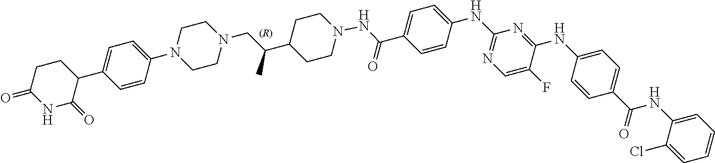 |
| 80 | 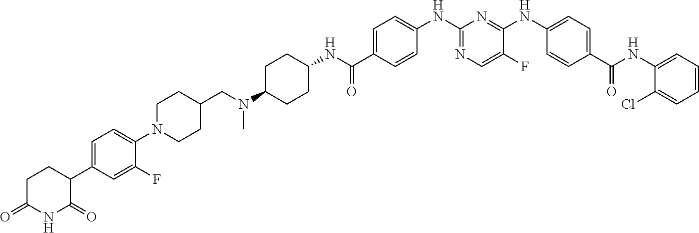 [SMILES: CN(CC1CCN(c2ccc(C3CCC(=O)NC3=O)cc2F)CC1)[C@H]1CC[C@H](NC(=O)c2ccc(Nc3ncc(F)c(Nc4ccc(C(=O)Nc5ccccc5Cl)cc4)n3)cc2)CC1] |
| 81 | 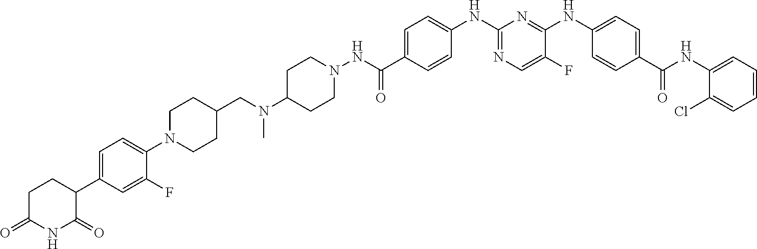 [SMILES: CN(CC1CCN(c2ccc(C3CCC(=O)NC3=O)cc2F)CC1)C1CCN(NC(=O)c2ccc(Nc3ncc(F)c(Nc4ccc(C(=O)Nc5ccccc5Cl)cc4)n3)cc2)CC1] |
| 82 | 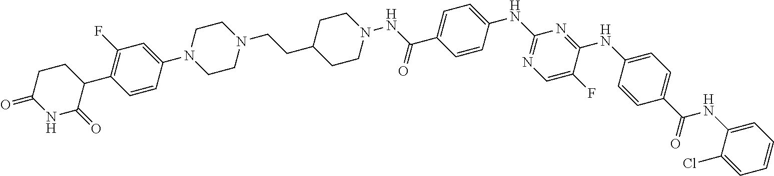 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2F)C(=O)N1] |
| 83 | 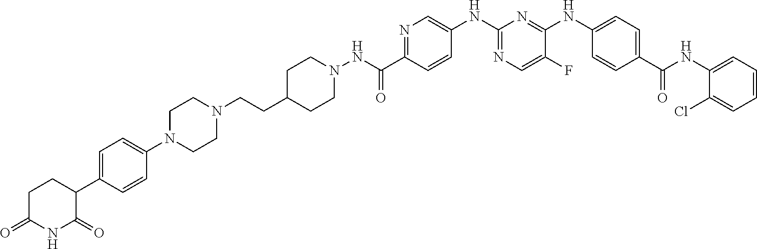 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cn5)CC4)CC3)cc2)C(=O)N1] |
| 84 | 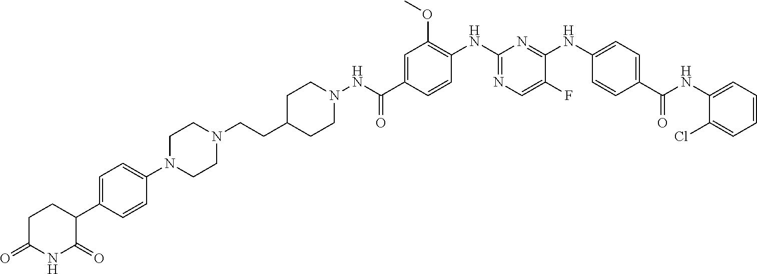 [SMILES: COc1cc(C(=O)NN2CCC(CCN3CCN(c4ccc(C5CCC(=O)NC5=O)cc4)CC3)CC2)ccc1Nc1ncc(F)c(Nc2ccc(C(=O)Nc3ccccc3Cl)cc2)n1] |
| 85 | 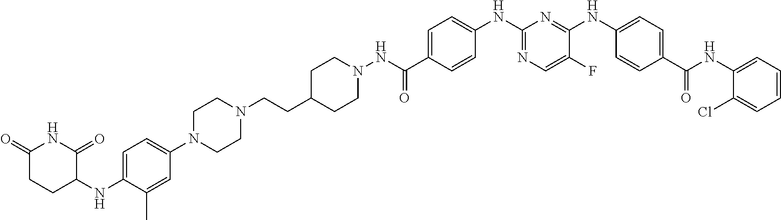 [SMILES: Cc1cc(N2CCN(CCC3CCN(NC(=O)c4ccc(Nc5ncc(F)c(Nc6ccc(C(=O)Nc7ccccc7Cl)cc6)n5)cc4)CC3)CC2)ccc1NC1CCC(=O)NC1=O] |
| 86 | 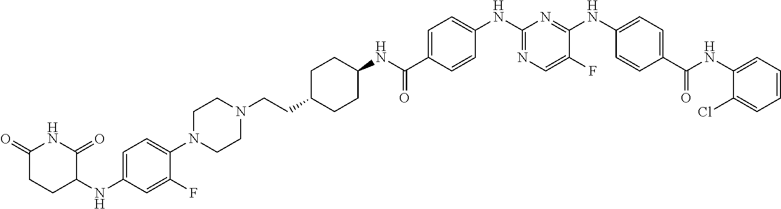 [SMILES: O=C1CCC(Nc2ccc(N3CCN(CC[C@H]4CC[C@H](NC(=O)c5ccc(Nc6ncc(F)c(Nc7ccc(C(=O)Nc8ccccc8Cl)cc7)n6)cc5)CC4)CC3)c(F)c2)C(=O)N1] |
| 87 | 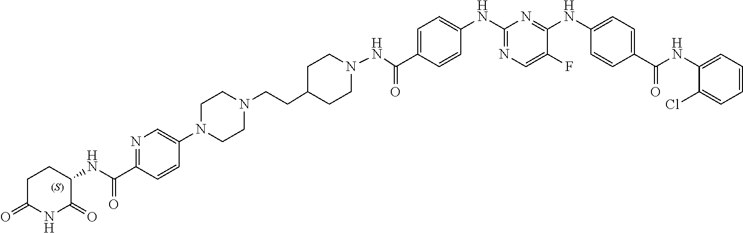 |
| 88 | 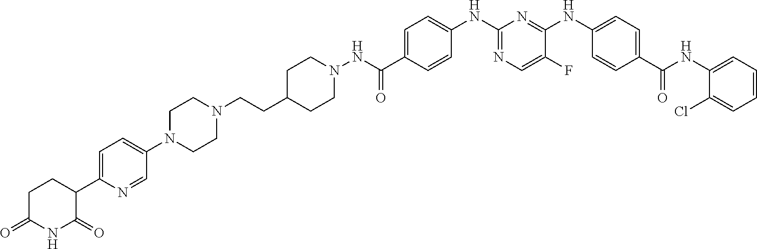 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cn2)C(=O)N1] |
| 89 | 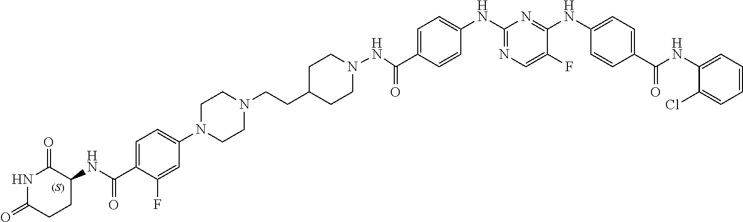 |
| 90 | 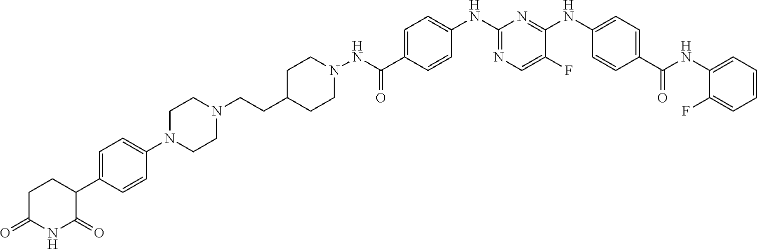 [SMILES: O=C1CCC(c2ccc(N3CCN(CCC4CCN(NC(=O)c5ccc(Nc6ncc(F)c(Nc7ccc(C(=O)Nc8ccccc8F)cc7)n6)cc5)CC4)CC3)cc2)C(=O)N1] |
| 91 | 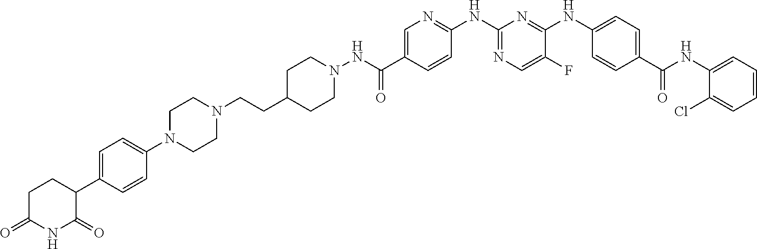 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)nc5)CC4)CC3)cc2)C(=O)N1] |
| 92 | 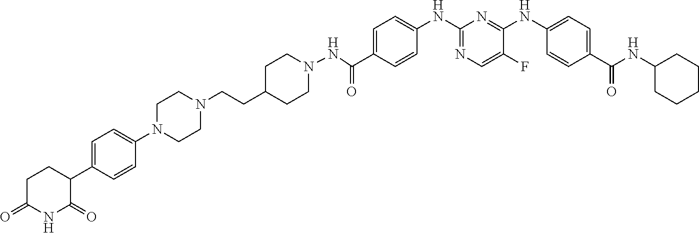 [SMILES: O=C1CCC(c2ccc(N3CCN(CCC4CCN(NC(=O)c5ccc(Nc6ncc(F)c(Nc7ccc(C(=O)NC8CCCCC8)cc7)n6)cc5)CC4)CC3)cc2)C(=O)N1] |
| 93 | 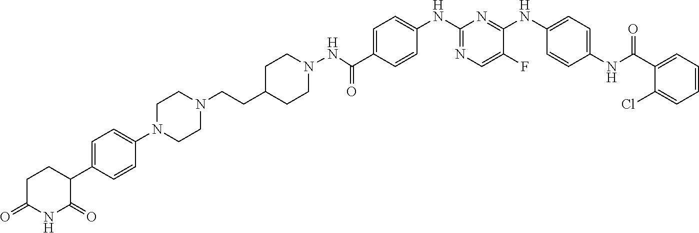 [SMILES: O=C1CCC(c2ccc(N3CCN(CCC4CCN(NC(=O)c5ccc(Nc6ncc(F)c(Nc7ccc(NC(=O)c8ccccc8Cl)cc7)n6)cc5)CC4)CC3)cc2)C(=O)N1] |
| 94 | 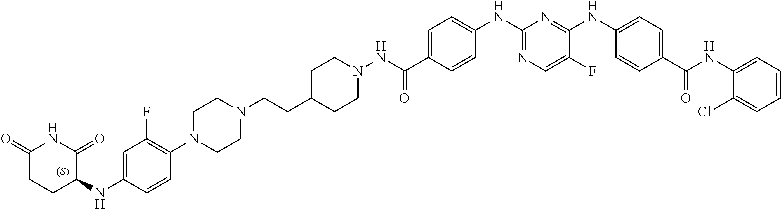 |
| 95 | 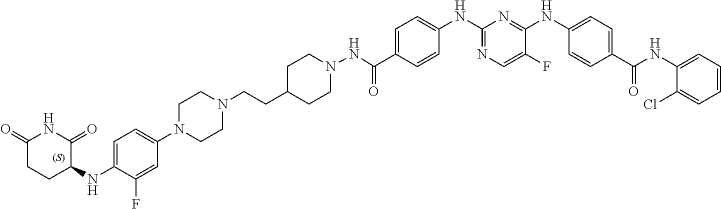 |
| 96 | 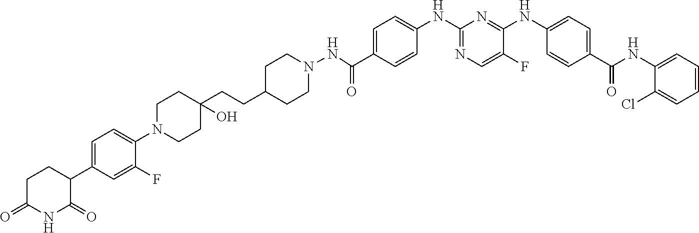 [SMILES: O=C1CCC(c2ccc(N3CCC(O)(CCC4CCN(NC(=O)c5ccc(Nc6ncc(F)c(Nc7ccc(C(=O)Nc8ccccc8Cl)cc7)n6)cc5)CC4)CC3)c(F)c2)C(=O)N1] |
| 97 | 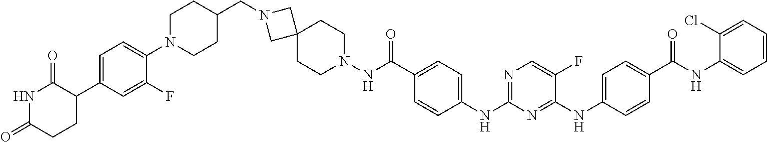 [SMILES: O=C1CCC(c2ccc(N3CCC(CN4CC5(CCN(NC(=O)c6ccc(Nc7ncc(F)c(Nc8ccc(C(=O)Nc9ccccc9Cl)cc8)n7)cc6)CC5)C4)CC3)c(F)c2)C(=O)N1] |
| 98 | 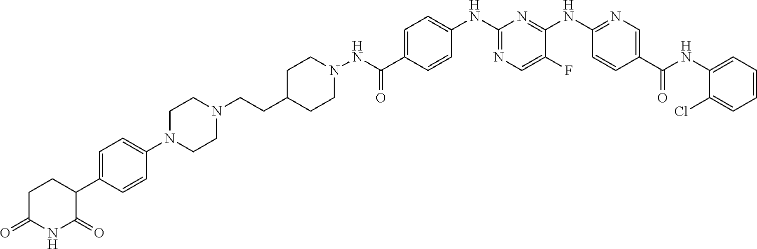 [SMILES: O=C1CCC(c2ccc(N3CCN(CCC4CCN(NC(=O)c5ccc(Nc6ncc(F)c(Nc7ccc(C(=O)Nc8ccccc8Cl)cn7)n6)cc5)CC4)CC3)cc2)C(=O)N1] |
| 99 | 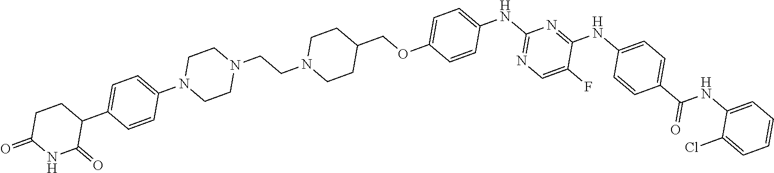 [SMILES: O=C1CCC(c2ccc(N3CCN(CCN4CCC(COc5ccc(Nc6ncc(F)c(Nc7ccc(C(=O)Nc8ccccc8Cl)cc7)n6)cc5)CC4)CC3)cc2)C(=O)N1] |
| 100 | 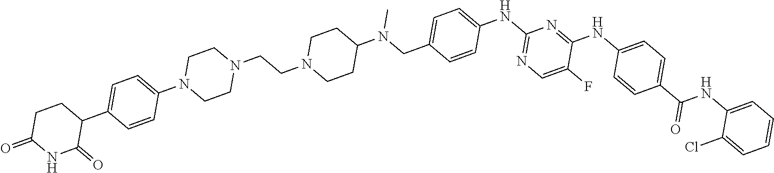 [SMILES: CN(Cc1ccc(Nc2ncc(F)c(Nc3ccc(C(=O)Nc4ccccc4Cl)cc3)n2)cc1)C1CCN(CCN2CCN(c3ccc(C4CCC(=O)NC4=O)cc3)CC2)CC1] |
| 101 | 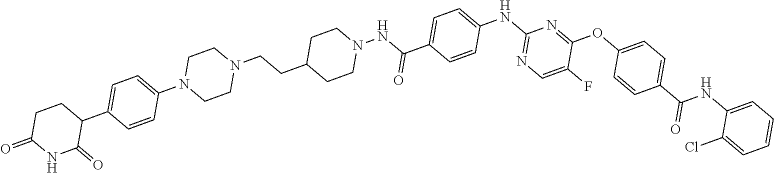 [SMILES: O=C1CCC(c2ccc(N3CCN(CCC4CCN(NC(=O)c5ccc(Nc6ncc(F)c(Oc7ccc(C(=O)Nc8ccccc8Cl)cc7)n6)cc5)CC4)CC3)cc2)C(=O)N1] |
| 102 | 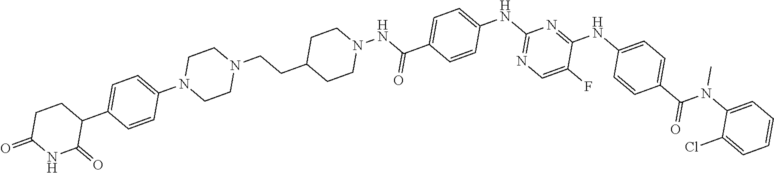 [SMILES: CN(C(=O)c1ccc(Nc2nc(Nc3ccc(C(=O)NN4CCC(CCN5CCN(c6ccc(C7CCC(=O)NC7=O)cc6)CC5)CC4)cc3)ncc2F)cc1)c1ccccc1Cl] |
| 103 | 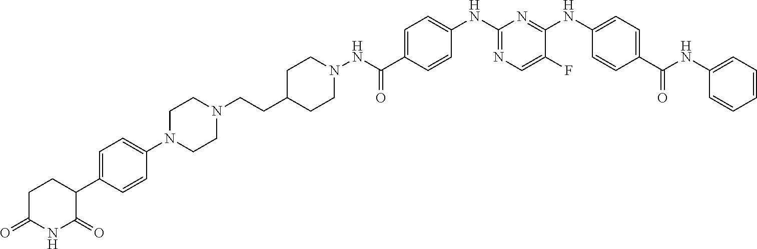 [SMILES: O=C1CCC(c2ccc(N3CCN(CCC4CCN(NC(=O)c5ccc(Nc6ncc(F)c(Nc7ccc(C(=O)Nc8ccccc8)cc7)n6)cc5)CC4)CC3)cc2)C(=O)N1] |
| 104 | 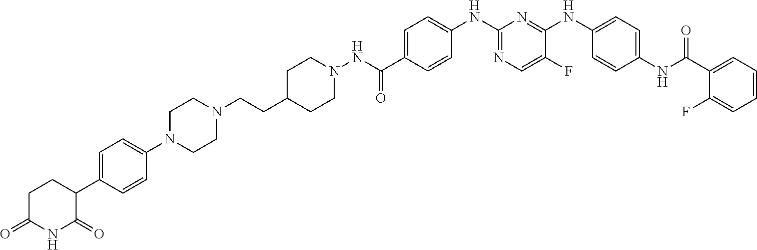 [SMILES: O=C1CCC(c2ccc(N3CCN(CCC4CCN(NC(=O)c5ccc(Nc6ncc(F)c(Nc7ccc(NC(=O)c8ccccc8F)cc7)n6)cc5)CC4)CC3)cc2)C(=O)N1] |
| 105 | 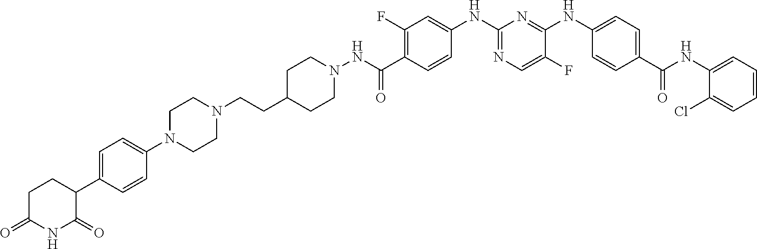 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5F)CC4)CC3)cc2)C(=O)N1] |
| 106 | 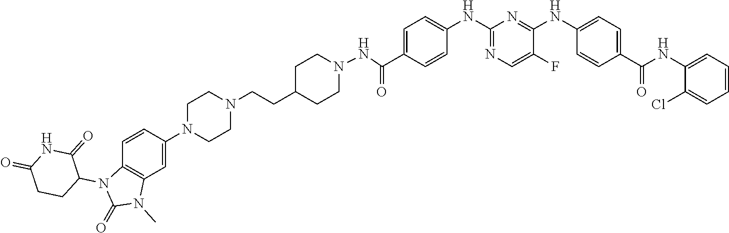 [SMILES: Cn1c(=O)n(C2CCC(=O)NC2=O)c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc21] |
| 107 | 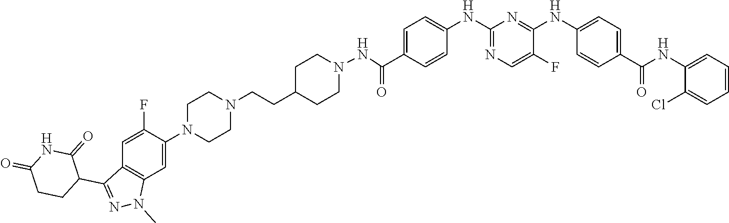 [SMILES: Cn1nc(C2CCC(=O)NC2=O)c2cc(F)c(N3CCN(CCC4CCN(NC(=O)c5ccc(Nc6ncc(F)c(Nc7ccc(C(=O)Nc8ccccc8Cl)cc7)n6)cc5)CC4)CC3)cc21] |
| 108 | 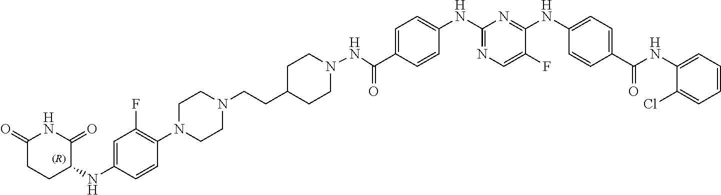 |
| 109 | 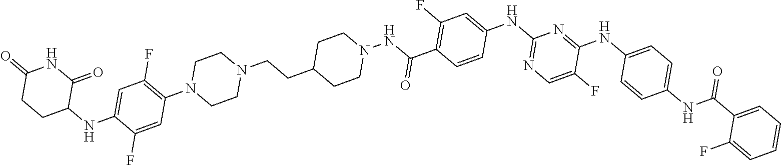 [SMILES: O=C1CCC(Nc2cc(F)c(N3CCN(CCC4CCN(NC(=O)c5ccc(Nc6ncc(F)c(Nc7ccc(NC(=O)c8ccccc8F)cc7)n6)cc5F)CC4)CC3)cc2F)C(=O)N1] |
| 110 | 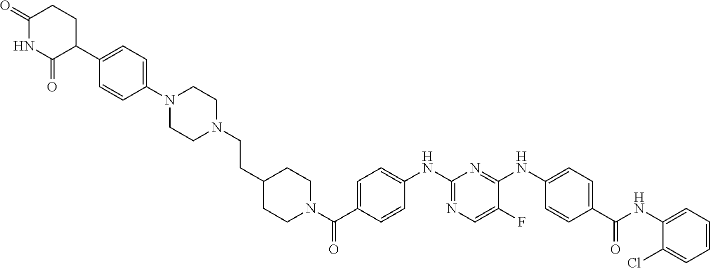 [SMILES: O=C1CCC(c2ccc(N3CCN(CCC4CCN(C(=O)c5ccc(Nc6ncc(F)c(Nc7ccc(C(=O)Nc8ccccc8Cl)cc7)n6)cc5)CC4)CC3)cc2)C(=O)N1] |
| 111 | 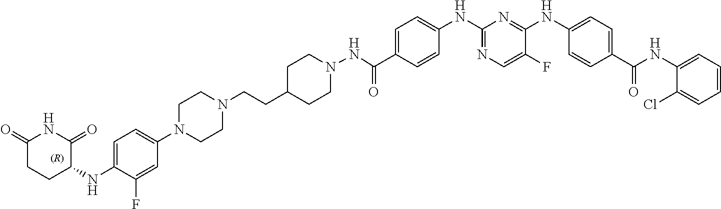 |
| 112 | 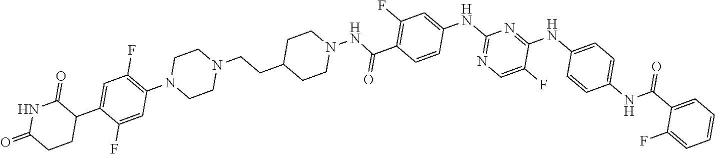 [SMILES: O=C1CCC(c2cc(F)c(N3CCN(CCC4CCN(NC(=O)c5ccc(Nc6ncc(F)c(Nc7ccc(NC(=O)c8ccccc8F)cc7)n6)cc5F)CC4)CC3)cc2F)C(=O)N1] |
| 113 | 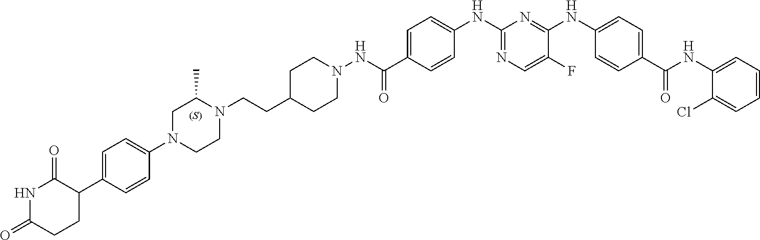 |
| 114 | 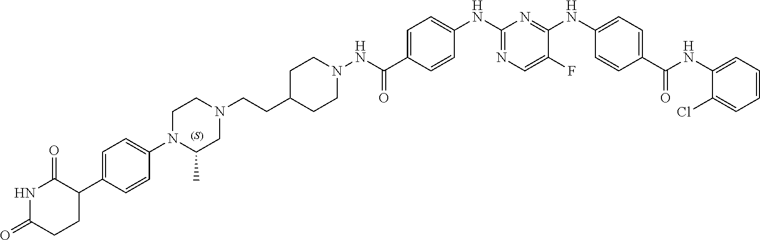 |
| 115 | 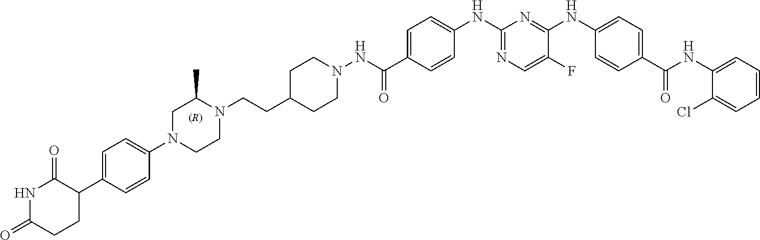 |
| 116 | 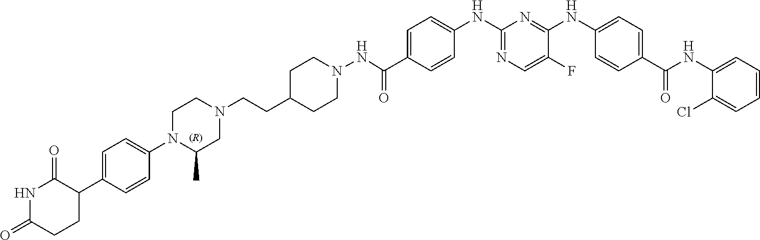 |
| 117 | 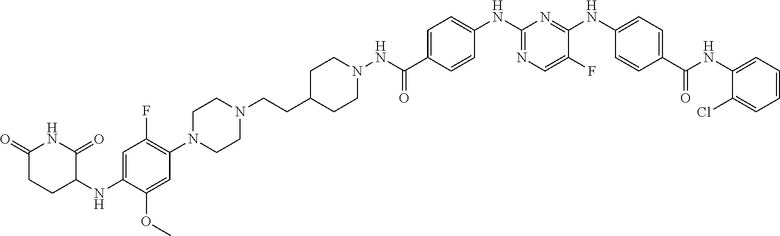 [SMILES: COc1cc(N2CCN(CCC3CCN(NC(=O)c4ccc(Nc5ncc(F)c(Nc6ccc(C(=O)Nc7ccccc7Cl)cc6)n5)cc4)CC3)CC2)c(F)cc1NC1CCC(=O)NC1=O] |
| 118 | 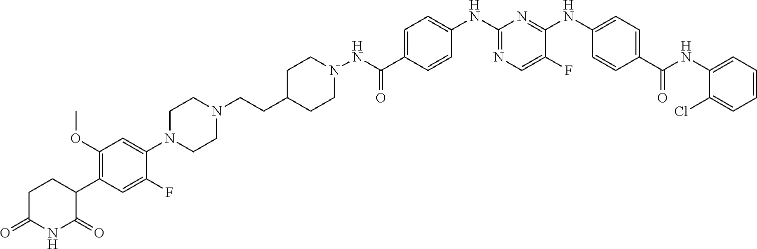 [SMILES: COc1cc(N2CCN(CCC3CCN(NC(=O)c4ccc(Nc5ncc(F)c(Nc6ccc(C(=O)Nc7ccccc7Cl)cc6)n5)cc4)CC3)CC2)c(F)cc1C1CCC(=O)NC1=O] |
| 119 | 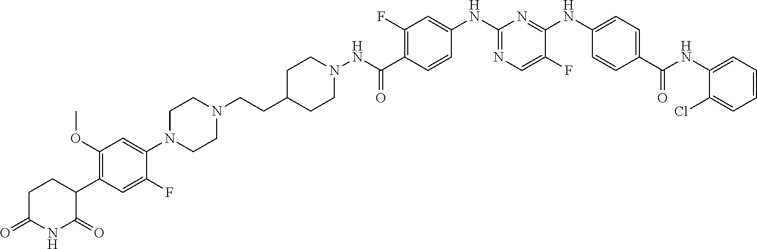 [SMILES: COc1cc(N2CCN(CCC3CCN(NC(=O)c4ccc(Nc5ncc(F)c(Nc6ccc(C(=O)Nc7ccccc7Cl)cc6)n5)cc4F)CC3)CC2)c(F)cc1C1CCC(=O)NC1=O] |
| 120 | 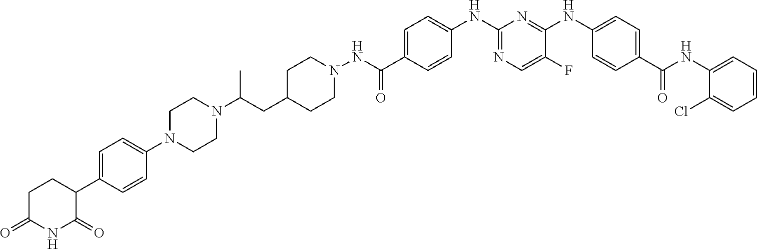 [SMILES: CC(CC1CCN(NC(=O)c2ccc(Nc3ncc(F)c(Nc4ccc(C(=O)Nc5ccccc5Cl)cc4)n3)cc2)CC1)N1CCN(c2ccc(C3CCC(=O)NC3=O)cc2)CC1] |
| 121 | 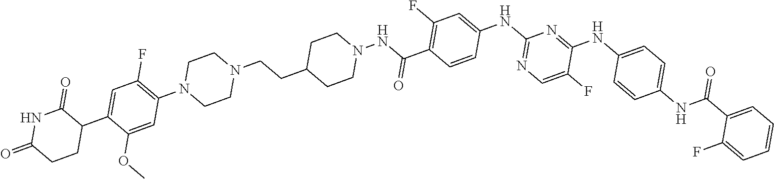 [SMILES: COc1cc(N2CCN(CCC3CCN(NC(=O)c4ccc(Nc5ncc(F)c(Nc6ccc(NC(=O)c7ccccc7F)cc6)n5)cc4F)CC3)CC2)c(F)cc1C1CCC(=O)NC1=O] |
| 122 | 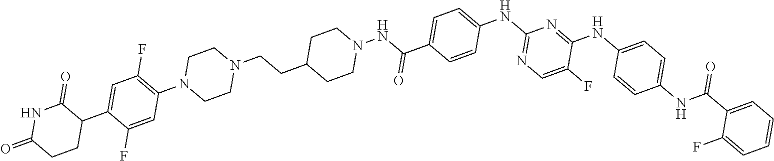 [SMILES: O=C1CCC(c2cc(F)c(N3CCN(CCC4CCN(NC(=O)c5ccc(Nc6ncc(F)c(Nc7ccc(NC(=O)c8ccccc8F)cc7)n6)cc5)CC4)CC3)cc2F)C(=O)N1] |
| 123 | 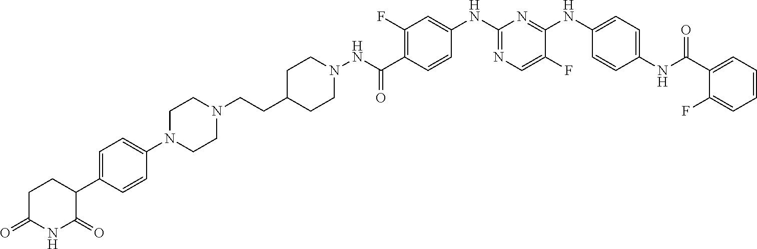 [SMILES: O=C1CCC(c2ccc(N3CCN(CCC4CCN(NC(=O)c5ccc(Nc6ncc(F)c(Nc7ccc(NC(=O)c8ccccc8F)cc7)n6)cc5F)CC4)CC3)cc2)C(=O)N1] |

The compounds of the present invention were purified according to the following method and the structure was analyzed.

### Instruments

LCMS: Shimadzu LCMS-2020
HPLC: Agilent 1260 II LC, Agilent 1200/G6410B
NMR: BRUKER AVANCE/400 MHZ

### LCMS Analysis

LCMS data were recorded with Shimadzu LCMS-2020 equipped with an ESI (Electron Spray Ionization) device. 0.0375% TFA in water (solvent A) and 0.01875% TFA in ACN (solvent B) were used as mobile phases. As a column, Kinetex EVO C18 (2.1×30 mm, 5 μm) or HALO C18 (3.0×30 mm, 2.7 μm) were used.

### HPLC Analysis

In HPLC analysis, Agilent 1260 II LC or Agilent 1200/G6410B were used. 0.0375% TFA in water (solvent A) and 0.01875% TFA in ACN (solvent B) were used as the mobile phase. As a column, Zobrax Eclipse Plus C18 (4.6×150 mm, 3.5 μm) or YMC ODS A (4.6×150 mm, 3 μm) were used.

### NMR Analysis

¹H NMR spectrum was recorded with Bruker AVANCE III 400 MHz/5 mm Probe (BBO).

### Example 1. Synthesis of N-(2-chlorophenyl)-4-((2-((4-(2-(4-((1-(4-(2,6-dioxopiperidin-3-yl)phenyl)piperidin-4-yl)methyl)piperazin-1-yl)-2-oxoethyl)phenyl)amino)-5-fluoropyrimidin-4-yl)amino)benzamide (Compound 1)

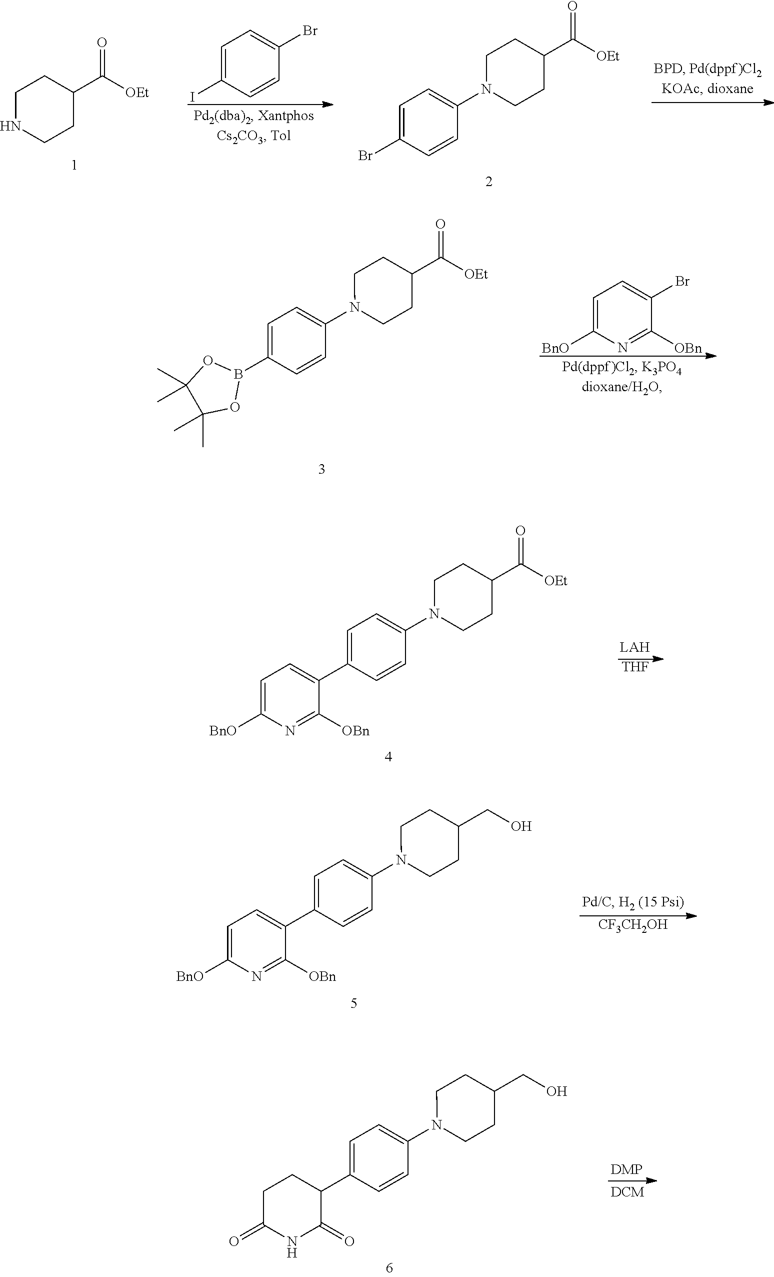

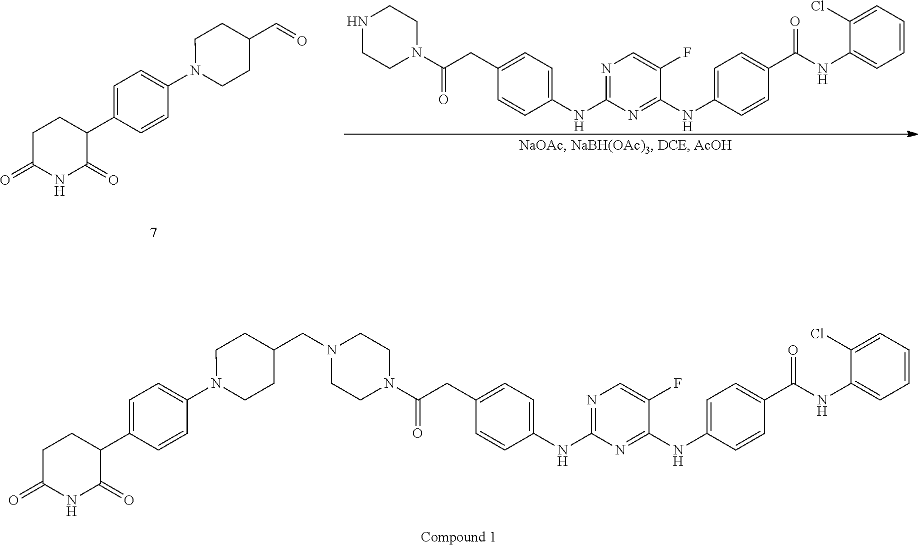 [SMILES: O=CC1CCN(c2ccc(C3CCC(=O)NC3=O)cc2)CC1 ; O=C(Nc1ccccc1Cl)c1ccc(Nc2nc(Nc3ccc(CC(=O)N4CCNCC4)cc3)ncc2F)cc1 ; O=C1CCC(c2ccc(N3CCC(CN4CCN(C(=O)Cc5ccc(Nc6ncc(F)c(Nc7ccc(C(=O)Nc8ccccc8Cl)cc7)n6)cc5)CC4)CC3)cc2)C(=O)N1]

### Step 1. Synthesis of ethyl 1-(4-bromophenyl)piperidine-4-carboxylate (2)

To a solution of 1-bromo-4-iodobenzene (9.00 g, 31.80 mmol) and ethyl piperidine-4-carboxylate (5 g, 31.80 mmol, 4.90 mL) in toluene (50 mL) were added Pd₂(dba)₃ (582.48 mg, 636.00 μmol), Xantphos (552.08 mg, 954.00 μmol) and Cs₂CO₃ (31.09 g, 95.40 mmol) under N₂, the mixture was stirred at 100° C. for 16 h. LCMS showed a major peak with desired mass. The mixture was filtered and the filtrate was concentrated under vacuum to afford ethyl 1-(4-bromophenyl)piperidine-4-carboxylate (7.7 g, 23.92 mmol, 75.23% yield, 97% purity) as yellow oil. MS(M+H)⁺=313.0.

### Step 2. Synthesis of ethyl 1-(4-(4,4,5,5-tetramethyl-1,3,2-dioxaborolan-2-yl)phenyl)piperidine-4-carboxylate (3)

To a solution of ethyl 1-(4-bromophenyl)piperidine-4-carboxylate (3 g, 9.61 mmol) in dioxane (30 mL) were added BPD (3.66 g, 14.41 mmol), KOAc (1.89 g, 19.22 mmol) and Pd(dppf)Cl₂ (351.56 mg, 480.46 μmol) under N₂. The resulting mixture was stirred at 100° C. for 16 h. LCMS showed a major peak with desired mass. The mixture was filtered and the filtrate was concentrated under vacuum. The crude product was purified by flash silica gel chromatography (Biotage, 10 g SepaFlash® Silica Flash Column, Eluent of 2~10% EtOAc/Petroleum ether gradient @ 50 mL/min) to afford ethyl 1-(4-(4,4,5,5-tetramethyl-1,3,2-dioxaborolan-2-yl)phenyl)piperidine-4-carboxylate (2.9 g, 5.49 mmol, 57.12% yield, 68% purity) as a yellow solid. MS(M+H)⁺=360.2.

### Step 3. Synthesis of ethyl 1-(4-(2,6-bis(benzyloxy)pyridin-3-yl)phenyl)piperidine-4-carboxylate (4)

To a solution of ethyl 1-(4-(4,4,5,5-tetramethyl-1,3,2-dioxaborolan-2-yl)phenyl)piperidine-4-carboxylate (2.9 g, 8.07 mmol) and 2,6-dibenzyloxy-3-bromo-pyridine (2.99 g, 8.07 mmol) in dioxane (20 mL) and H₂O (4 mL) were added K₃PO₄ (5.14 g, 24.22 mmol) and Pd(dppf)Cl₂ (295.32 mg, 403.60 μmol). The mixture was stirred at 90° C. for 16 h. LCMS showed 32% peak with desired mass. The mixture was filtered and the filtrate was diluted with water (30 mL), extracted with EtOAc (20 mL×3). The organic layer was dried over Na₂SO₄, filtered and concentrated under vacuum. The crude product was purified by flash silica gel chromatography (Biotage, 25 g SepaFlash® Silica Flash Column, Eluent of 4-70% EtOAc/Petroleum ether gradient @ 55 mL/min) to afford ethyl 1-(4-(2,6-bis(benzyloxy)pyridin-3-yl)phenyl)piperidine-4-carboxylate (0.9 g, 1.55 mmol, 19.20% yield, 90% purity) as yellow oil. MS(M+H)⁺=533.1.

### Step 4. Synthesis of (1-(4-(2,6-bis(benzyloxypyridin-3-yl)phenyl)piperidin-4-yl)methanol (5)

To a solution of ethyl 1-(4-(2,6-dibenzyloxy-3-pyridyl)phenyl)piperidine-4-carboxylate (0.9 g, 1.72 mmol) in THF (10 mL) was added LAH (92.80 mg, 2.45 mmol) under N₂. The mixture was stirred at 25° C. for 1 h. LCMS showed a major peak with desired mass. The reaction mixture was quenched with H₂O (0.09 mL), aqueous solution of NaOH (15%, 0.09 mL) and H₂O (0.27 mL) at 0° C. Then the mixture was filtered and washed with EtOAc. The filtrate was dried over Na₂SO₄ and concentrated under reduced pressured to afford (1-(4-(2,6-bis(benzyloxy)pyridin-3-yl)phenyl)piperidin-4-yl)methanol (700 mg, crude) as yellow oil. MS(M+H)⁺=481.2.

### Step 5. Synthesis of 3-(4-(4-(hydroxymethyl)piperidin-1-yl)phenyl)piperidine-2,6-dione (6)

To a solution of (1-(4-(2,6-bis(benzyloxy)pyridin-3-yl)phenyl)piperidin-4-yl)methanol (700 mg, 1.46 mmol) in CF₃CH₂OH (10 mL) was added Pd/C (300 mg, 10% purity) under H₂. The mixture was degassed with N₂ for 3 times and then stirred under H₂ (15 psi) at 25° C. for 16 h. LCMS showed a major peak with desired mass. The mixture was filtered and the filtrate was concentrated under vacuum to afford 3-(4-(4-(hydroxymethyl)piperidin-1-yl)phenyl)piperidine-2,6-dione (400 mg, crude) as yellow oil. MS(M+H)⁺=303.1.

### Step 6. Synthesis of 1-(4-(2,6-dioxopiperidin-3-yl)phenyl)piperidine-4-carbaldehyde (7)

To a solution of 3-(4-(4-(hydroxymethyl)piperidin-1-yl)phenyl)piperidine-2,6-dione (100 mg, 330.72 μmol) in DCM (2 mL) was added DMP (168.33 mg, 396.87 μmol, 122.87 μL), the mixture was stirred at 25° C. for 1 h. TLC (petroleum ether:EtOAc=1:1) showed most of the starting material consumed and a new spot was formed. The reaction mixture was filtered and the filtrate was concentrated to afford 1-(4-(2,6-dioxopiperidin-3-yl)phenyl)piperidine-4-carbaldehyde (120 mg, crude) as brown oil. The crude product was used for the next step directly. MS(M+H)⁺=301.1.

### Step 7. Synthesis of N-(2-chlorophenyl)-4-((2-((4-(2-(4-((1-(4-(2,6-dioxopiperidin-3-yl)phenyl)piperidin-4-yl) methyl)piperazin-1-yl)-2-oxoethyl)phenyl)amino)-5-fluoropyrimidin-4-yl)amino)benzamide (Compound 1)

To a solution of N-(2-chlorophenyl)-4-((5-fluoro-2-((4-(2-oxo-2-(piperazin-1-yl)ethyl)phenyl)amino)pyrimidin-4-yl)amino)benzamide (90 mg, 150.88 μmol) in DCE (6 mL) was added NaOAc (12.38 mg, 150.88 μmol). The mixture was stirred at 25° C. for 10 min, then AcOH (9.06 mg, 150.88 μmol, 8.63 μL) and 1-(4-(2,6-dioxopiperidin-3-yl)phenyl)piperidine-4-carbaldehyde (67.98 mg, 226.33 mol) were added. After stirring at 25° C. for 0.5 h, NaBH(OAc)₃ (47.97 mg, 226.33 μmol) was added. The resulting mixture was stirred at 25° C. for 2.5 h. LCMS showed 40% peak with desired mass. The mixture was diluted with water (10 mL) and extracted with EtOAc (5 mL×3). The combined organic layer was dried over Na₂SO₄, filtered and concentrated under vacuum. The crude product was purified by flash silica gel chromatography (Biotage, 4 g SepaFlash® Silica Flash Column, Eluent of 4~100% EtOAc/Petroleum ether gradient @ 50 mL/min), followed prep-HPLC(column: Waters Xbridge 150*25 mm*5 um; mobile phase: [water(NH₄HCO₃)-ACN]; B %: 40%-70%, 8 min) and lyophilized to afford N-(2-chlorophenyl)-4-((2-((4-(2-(4-((1-(4-(2,6-dioxopiperidin-3-yl)phenyl)piperidin-4-yl) methyl)piperazin-1-yl)-2-oxoethyl)phenyl)amino)-5-fluoropyrimidin-4-yl)amino)benzamide (12.5 mg, 13.63 μmol, 9.04% yield, 92.1% purity) as white powder. MS(M+H)⁺=844.3.

¹H NMR (400 MHz, DMSO-d₆) δ=10.77 (s, 1H), 9.89 (s, 1H), 9.66 (s, 1H), 9.29 (s, 1H), 8.19 (d, J=3.7 Hz, 1H), 8.07-8.01 (m, 2H), 8.00-7.95 (m, 2H), 7.66 (dd, J=1.3, 8.1 Hz, 1H), 7.61 (d, J=8.6 Hz, 2H), 7.57 (dd, J=1.3, 8.1 Hz, 1H), 7.43-7.37 (m, 1H), 7.32-7.27 (m, 1H), 7.13 (d, J=8.4 Hz, 2H), 7.02 (d, J=8.7 Hz, 2H), 6.86 (d, J=8.8 Hz, 2H), 3.71 (dd, J=5.1, 10.6 Hz, 1H), 3.68-3.58 (m, 4H), 3.49-3.42 (m, 4H), 2.67-2.55 (m, 3H), 2.30-2.22 (m, 4H), 2.20-2.12 (m, 3H), 2.05-1.95 (m, 1H), 1.80-1.72 (m, 2H), 1.67-1.55 (m, 1H), 1.28-0.99 (m, 3H).

### Example 2. Synthesis of N-(2-chlorophenyl)-4-((2-((4-(2-(4-((1-(3-(2,6-dioxopiperidin-3-yl)phenyl)piperidin-4-yl)methyl)piperazin-1-yl)-2-oxoethyl)phenyl)amino)-5-fluoropyrimidin-4-yl)amino)benzamide (Compound 2)

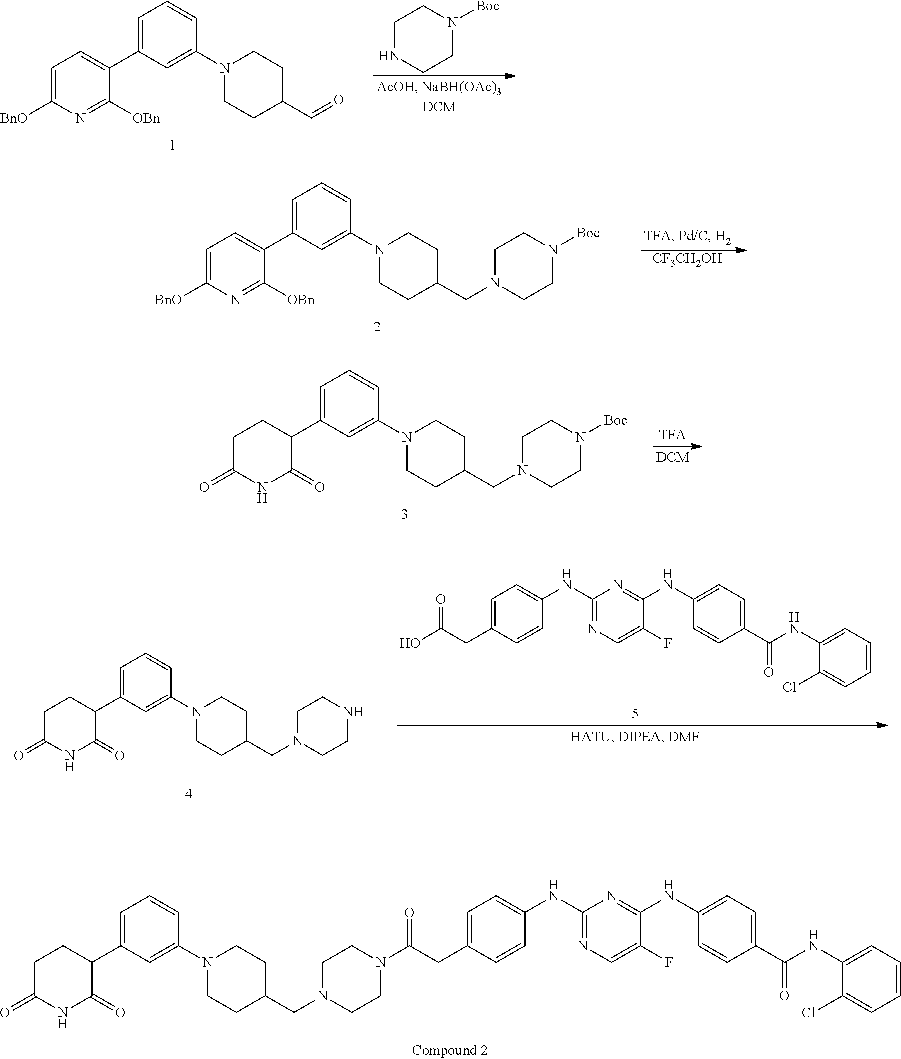

### Step 1. Synthesis of tert-butyl 4-((1-(3-(2,6-bis(benzyloxy)pyridin-3-yl)phenyl)piperidin-4-yl)methyl)piperazine-1-carboxylate (2)

To a solution of 1-(3-(2,6-bis(benzyloxy)pyridin-3-yl)phenyl)piperidine-4-carbaldehyde (0.3 g, 626.85 mol) and tert-butyl piperazine-1-carboxylate (116.75 mg, 626.85 μmol) in DCM (8 mL) were added AcOH (37.64 mg, 626.85 μmol, 35.85 μL) at 20° C. Then NaBH(OAc)₃ (398.57 mg, 1.88 mmol) was slowly added at 20° C. and the resulting mixture was stirred at 20° C. for 12 h. LCMS showed 1-(3-(2,6-bis(benzyloxy)pyridin-3-yl)phenyl)piperidine-4-carbaldehyde was consumed completely and 20% peak with desired mass. The reaction mixture was diluted with H₂O (15 mL) and extracted with DCM (10 mL×3). The organic layer was washed with saturated NaHCO₃ (10 mL×3), dried over Na₂SO₄, filtered and concentrated. The residue was purified by flash silica gel chromatography (10 g SepaFlash® Silica Flash Column, Eluent of 0-33% EtOAc/Petroleum ether gradient @ 100 mL/min) to afford tert-butyl 4-((1-(3-(2,6-bis(benzyloxy)pyridin-3-yl)phenyl)piperidin-4-yl)methyl)piperazine-1-carboxylate (183 mg, 262.30 μmol, 41.84% yield, 93% purity) as a yellow oil. MS(M+H)⁺=649.3.

### Step 2. Synthesis of tert-butyl 4-((1-(3-(2,6-dioxopiperidin-3-yl)phenyl)piperidin-4-yl)methyl)piperazine-1-carboxylate (3)

To a solution of tert-butyl 4-((1-(3-(2,6-bis(benzyloxy)pyridin-3-yl)phenyl)piperidin-4-yl)methyl)piperazine-1-carboxylate (183 mg, 282.05 μmol) in CF₃CH₂OH (5 mL) was added Pd/C (0.1 g, 10% purity) and TFA (32.16 mg, 282.05 μmol, 20.88 μL) under H₂ atmosphere. The suspension was degassed and purged with H₂ for 3 times. The mixture was stirred under H₂ (15 Psi) at 20° C. for 12 h. LCMS showed starting material was consumed completely and peak with desired mass. The reaction mixture was diluted with CF₃CH₂OH (15 mL) and filtered. The filtrate was concentrated in vacuum to afford tert-butyl 4-((1-(3-(2,6-dioxopiperidin-3-yl)phenyl)piperidin-4-yl)methyl)piperazine-1-carboxylate (133 mg, crude) as a colorless oil. MS(M+H)⁺=471.3.

### Step 3. Synthesis of 3-(3-(4-(piperazin-1-ylmethyl)piperidin-1-yl)phenyl)piperidine-2,6-dione (4)

To a solution of tert-butyl 4-((1-(3-(2,6-dioxopiperidin-3-yl)phenyl)piperidin-4-yl)methyl)piperazine-1-carboxylate (133 mg, 282.62 μmol) in DCM (2 mL) was added TFA (161.12 mg, 1.41 mmol, 104.63 μL) at 20° C. and the resulting mixture was stirred at 20° C. for 0.5 h. LCMS showed starting material was consumed completely and peak with desired mass. The reaction mixture was concentrated in vacuum to afford 3-(3-(4-(piperazin-1-ylmethyl)piperidin-1-yl)phenyl)piperidine-2,6-dione (137 mg, crude, TFA) as a yellow oil. MS(M+H)⁺=371.3.

### Step 4. Synthesis of N-(2-chlorophenyl)-4-((2-((4-(2-(4-((1-(3-(2,6-dioxopiperidin-3-yl)phenyl)piperidin-4-yl) methyl)piperazin-1-yl)-2-oxoethyl)phenyl)amino)-5-fluoropyrimidin-4-yl)amino)benzamide (Compound 2)

To a solution of 2-(4-((4-((4-((2-chlorophenyl)carbamoyl)phenyl)amino)-5-fluoropyrimidin-2-yl)amino)phenyl)acetic acid (100 mg, 203.29 μmol) in DMF (2 mL) was added HATU (85.03 mg, 223.62 μmol) and DIPEA (52.55 mg, 406.59 μmol, 70.82 μL). The mixture was stirred at 20° C. for 10 min and a solution of 3-(3-(4-(piperazin-1-ylmethyl)piperidin-1-yl)phenyl)piperidine-2,6-dione (137 mg, 282.76 μmol, TFA) in DMF (2 mL) with DIPEA (105.10 mg, 813.17 μmol, 141.64 μL) was added and the resulting mixture was stirred at 20° C. for 1 h. LCMS showed starting material was consumed completely and 25% peak with desired mass. The reaction mixture was diluted with H₂O (15 mL) and extracted with EtOAc (15 mL×3). The organic layer was washed with brine (15 mL×3), dried over Na₂SO₄, filtered and concentrated. The residue was purified by prep-TLC (SiO₂, DCM:MeOH=10:1), followed by prep-HPLC (column: Waters Xbridge 150*25 mm*5 um; mobile phase: [water (NH₄HCO₃)-ACN]; B %: 47%-77%, 8 min) and lyophilized to afford N-(2-chlorophenyl)-4-((2-((4-(2-(4-((1-(3-(2,6-dioxopiperidin-3-yl)phenyl)piperidin-4-yl) methyl)piperazin-1-yl)-2-oxoethyl)phenyl)amino)-5-fluoropyrimidin-4-yl)amino)benzamide (23.5 mg, 26.44 μmol, 13.01% yield, 95% purity) as a white solid. MS(M+H)⁺=844.3.

¹H NMR (400 MHz, DMSO-d₆) δ=10.78 (br s, 1H), 9.88 (s, 1H), 9.65 (s, 1H), 9.28 (s, 1H), 8.18 (d, J=3.7 Hz, 1H), 8.07-7.94 (m, 4H), 7.70-7.53 (m, 4H), 7.39 (dt, J=1.2, 7.6 Hz, 1H), 7.32-7.25 (m, 1H), 7.17-7.09 (m, 3H), 6.82-6.74 (m, 2H), 6.57 (d, J=7.6 Hz, 1H), 3.74 (dd, J=5.0, 11.1 Hz, 1H), 3.69-3.58 (m, 4H), 3.50-3.43 (m, 4H), 2.68-2.55 (m, 3H), 2.48-2.41 (m, 1H), 2.31-2.20 (m, 4H), 2.20-2.14 (m, 1H), 2.14-2.07 (m, 2H), 2.06-1.96 (m, 1H), 1.78-1.69 (m, 2H), 1.68-1.54 (m, 1H), 1.25-1.08 (m, 2H).

### Example 3. Synthesis of N-(2-chlorophenyl)-4-((2-((4-(4-((4-(4-(2,6-dioxopiperidin-3-yl)phenyl)piperazin-1-yl)methyl)piperidine-1-carbonyl)phenyl)amino)-5-fluoropyrimidin-4-yl)amino)benzamide (Compound 3)

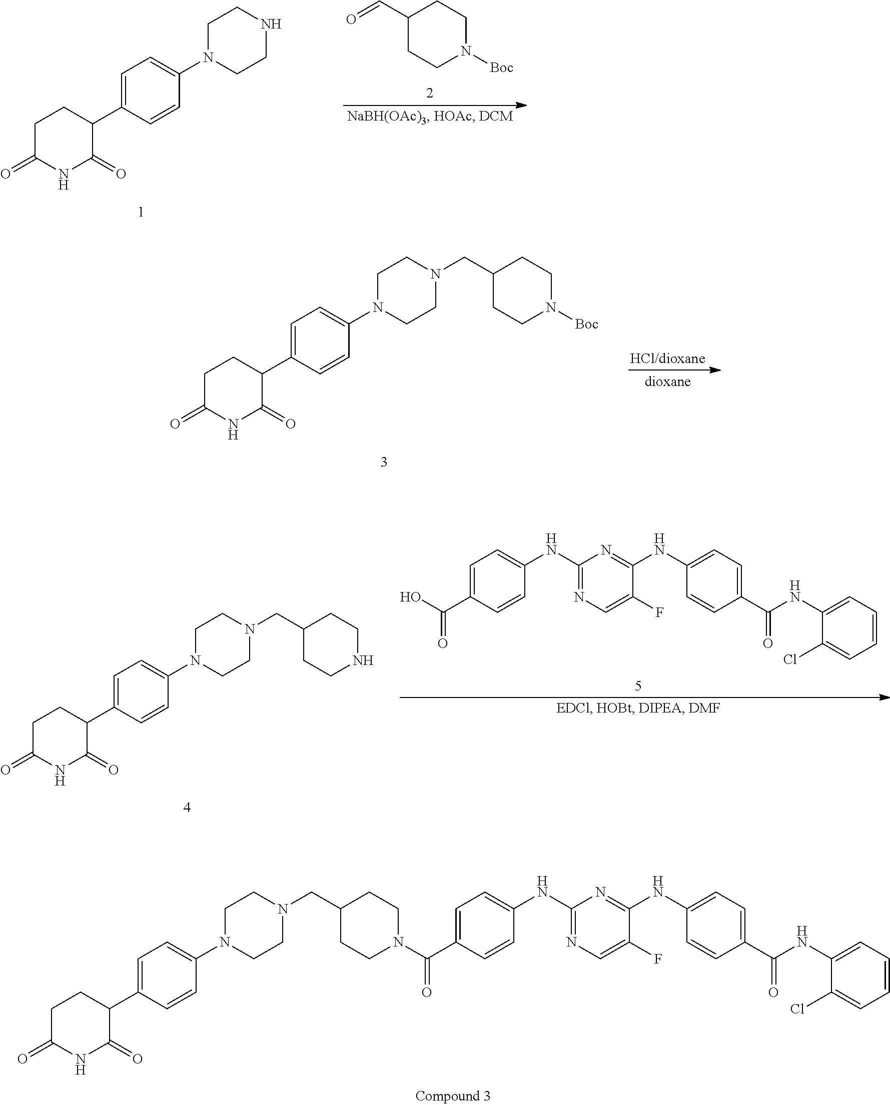 [SMILES: O=C1CCC(c2ccc(N3CCNCC3)cc2)C(=O)N1 ; CC(C)(C)OC(=O)N1CCC(C=O)CC1 ; CC(C)(C)OC(=O)N1CCC(CN2CCN(c3ccc(C4CCC(=O)NC4=O)cc3)CC2)CC1 ; O=C1CCC(c2ccc(N3CCN(CC4CCNCC4)CC3)cc2)C(=O)N1 ; O=C(O)c1ccc(Nc2ncc(F)c(Nc3ccc(C(=O)Nc4ccccc4Cl)cc3)n2)cc1 ; O=C1CCC(c2ccc(N3CCN(CC4CCN(C(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of tert-butyl 4-((4-(4-(2,6-dioxopiperidin-3-yl)phenyl)piperazin-1-yl)methyl)piperidine-1-carboxylate (3)

To a solution of 3-(4-(piperazin-1-yl)phenyl)piperidine-2,6-dione (0.2 g, 645.60 μmol, HCl) in DCM (5 mL) were added tert-butyl 4-formylpiperidine-1-carboxylate (275.38 mg, 1.29 mmol) and HOAc (1.94 mg, 32.28 μmol, 1.85 μL). The mixture was stirred at 25° C. for 0.5 h. Then NaBH(OAc)₃ (273.66 mg, 1.29 mmol) was added and the resulting mixture was stirred at 25° C. for 2 hrs. LCMS showed a main peak with desired mass. The mixture was diluted with water (30 ml) and extracted with DCM (20 mL×5). The combined organic layers were washed with saturated brine (20 mL), dried over anhydrous Na₂SO₄, filtered and concentrated under reduced pressure. The residue was purified by flash silica gel chromatography (ISCO®; 20 g SepaFlash® Silica Flash Column, Eluent of 0~40% petroleum ether:EtOAc/EtOH (V/V=5:1) gradient @ 80 mL/min) to afford tert-butyl 4-((4-(4-(2,6-dioxopiperidin-3-yl)phenyl)piperazin-1-yl)methyl)piperidine-1-carboxylate (220 mg, 455.33 μmol, 70.53% yield, 97.4% purity) as a white solid. MS(M+H)⁺=471.3.

### Step 2. Synthesis of 3-(4-(4-(piperidin-4-ylmethyl)piperazin-1-yl)phenyl)piperidine-2,6-dione (4)

To a solution of tert-butyl 4-((4-(4-(2,6-dioxopiperidin-3-yl)phenyl)piperazin-1-yl)methyl)piperidine-1-carboxylate (220 mg, 467.49 μmol) in dioxane (5 mL) was added HCl/dioxane (4 M, 5 mL). The mixture was stirred at 25° C. for 1 h. LCMS showed the starting material was consumed completely. The mixture was concentrated under reduced pressure to afford 3-(4-(4-(piperidin-4-ylmethyl)piperazin-1-yl)phenyl)piperidine-2,6-dione (200 mg, crude, HCl) as a white solid. MS(M+H)⁺=371.2.

### Step 3. Synthesis of N-(2-chlorophenyl)-4-((2-((4-(4-((4-(4-(2,6-dioxopiperidin-3-yl)phenyl)piperazin-1-yl)methyl)piperidine-1-carbonyl)phenyl)amino)-5-fluoropyrimidin-4-yl)amino)benzamide (Compound 3)

To a solution of 4-((4-((4-((2-chlorophenyl)carbamoyl)phenyl)amino)-5-fluoropyrimidin-2-yl)amino)benzoic acid (150.00 mg, 313.89 μmol) in DMF (5 mL) were added EDCI (90.26 mg, 470.84 μmol), DIPEA (162.27 mg, 1.26 mmol, 218.70 μL) and HOBt (63.62 mg, 470.84 μmol). The mixture was stirred at 25° C. for 0.5 h. Then 3-(4-(4-(piperidin-4-ylmethyl)piperazin-1-yl)phenyl)piperidine-2,6-dione (127.74 mg, 313.89 μmol, HCl) was added and the resulting mixture was stirred at 25° C. for 2 h. LCMS showed 67% peak with desired mass. The mixture was diluted with water (30 ml) and extracted with EtOAc (20 mL×5). The combined organic layers were washed with saturated brine (20 mL), dried over anhydrous Na₂SO₄, filtered and concentrated under reduced pressure. The residue was purified by prep-HPLC (column: Phenomenex luna C18 150×25 mm×10 μm; mobile phase: [water (FA)-ACN]; B %: 13%-43%, 10 min) and then prep-HPLC(column: Phenomenex Synergi Polar-RP 100×25 mm×4 μm; mobile phase: [water (TFA)-ACN]; B %: 24%-54%, 9 min) and lyophilized to afford N-(2-chlorophenyl)-4-((2-((4-(4-((4-(4-(2,6-dioxopiperidin-3-yl)phenyl)piperazin-1-yl)methyl)piperidine-1-carbonyl)phenyl)amino)-5-fluoropyrimidin-4-yl)amino)benzamide (44.2 mg, 45.54 μmol, 63.02% yield, 97.3% purity, TFA) as a white solid. MS(M+H)⁺=830.2.

¹H NMR (400 MHz, CD₃OD) δ=8.11 (d, J=4.2 Hz, 1H), 8.01-7.97 (m, 2H), 7.93-7.89 (m, 2H), 7.73 (dd, J=1.6, 7.9 Hz, 1H), 7.66 (d, J=8.6 Hz, 2H), 7.54 (dd, J=1.3, 7.9 Hz, 1H), 7.42-7.38 (m, 3H), 7.32-7.26 (m, 1H), 7.21 (d, J=8.7 Hz, 2H), 6.99 (d, J=8.8 Hz, 2H), 4.04-3.34 (m, 6H), 3.18-2.92 (m, 8H), 2.81-2.53 (m, 3H), 2.26-2.16 (m, 3H), 1.96-1.89 (m, 2H), 1.43-1.34 (m, 2H).

### Example 4. Synthesis of N-(2-chlorophenyl)-4-((2-((4-((4-(((1-(4-(2,6-dioxopiperidin-3-yl)phenyl)piperidin-4-yl)methyl)(methyl)amino)cyclohexyl)carbamoyl)phenyl)amino)-5-fluoropyrimidin-4-yl)amino)benzamide (Compound 4)

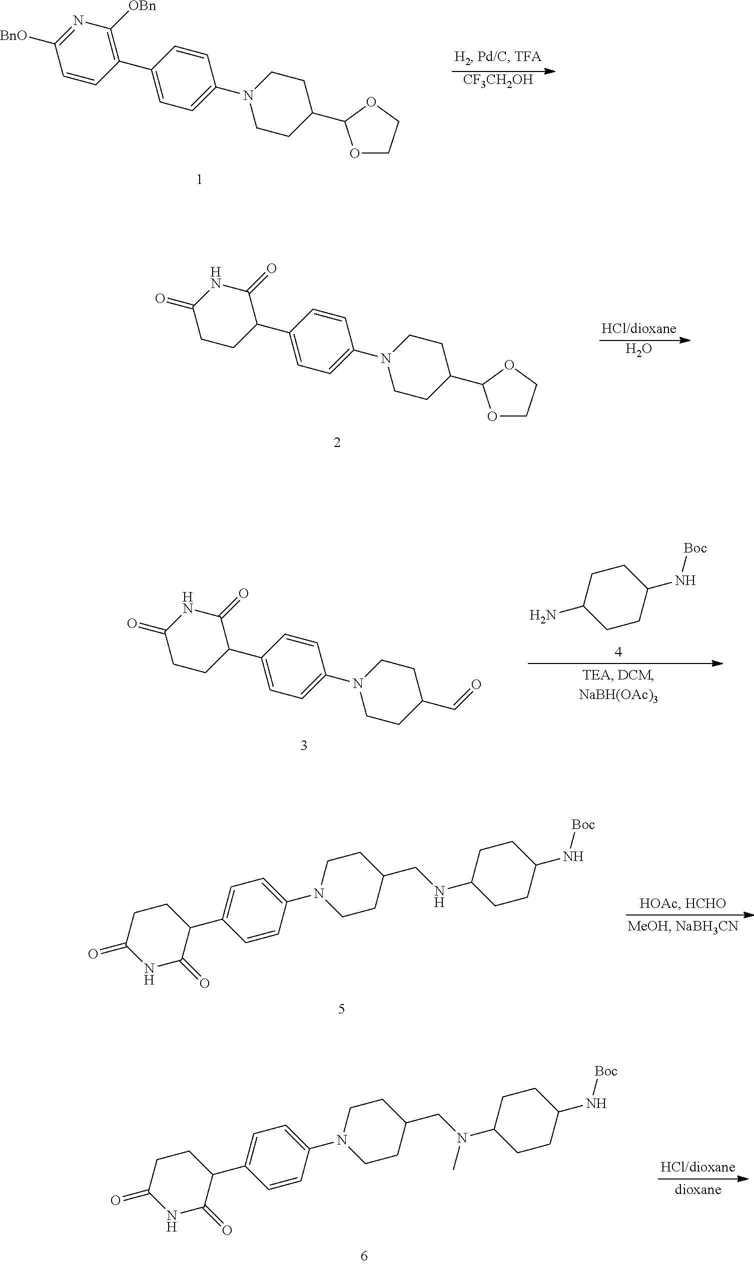

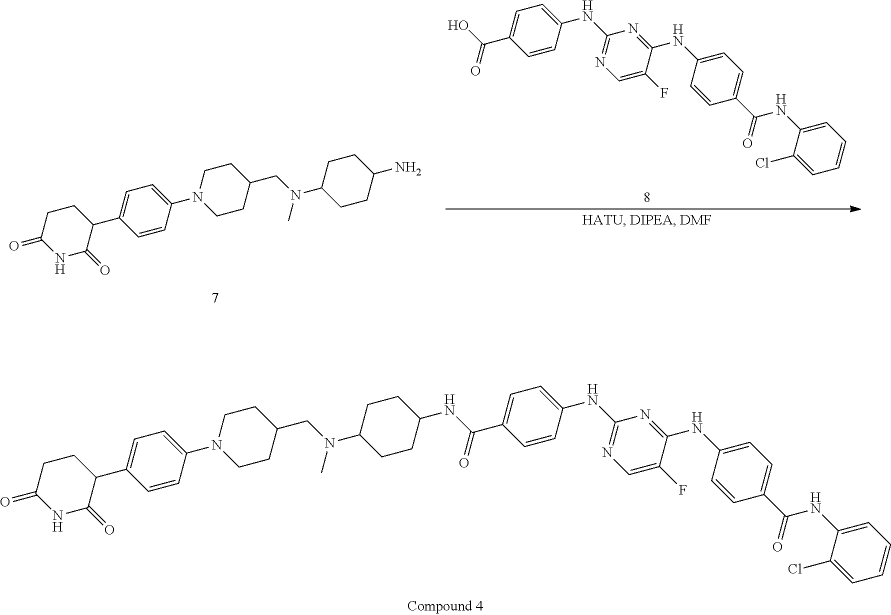 [SMILES: CN(CC1CCN(c2ccc(C3CCC(=O)NC3=O)cc2)CC1)C1CCC(N)CC1 ; O=C(O)c1ccc(Nc2ncc(F)c(Nc3ccc(C(=O)Nc4ccccc4Cl)cc3)n2)cc1 ; CN(CC1CCN(c2ccc(C3CCC(=O)NC3=O)cc2)CC1)C1CCC(NC(=O)c2ccc(Nc3ncc(F)c(Nc4ccc(C(=O)Nc5ccccc5Cl)cc4)n3)cc2)CC1]

### Step 1. Synthesis of 3-(4-(4-(1,3-dioxolan-2-yl)piperidin-1-yl)phenyl)piperidine-2,6-dione (2)

To a solution of 3-(4-(4-(1,3-dioxolan-2-yl)piperidin-1-yl)phenyl)-2,6-bis(benzyloxy)pyridine (0.5 g, 956.69 μmol) in CF₃CH₂OH (10 mL) was added TFA (153.50 mg, 1.35 mmol, 0.1 mL) and Pd/C (101.81 mg, 95.67 μmol, 10% purity) under H₂. The resulting mixture was stirred at 25° C. under H₂ (15 psi) for 24 h. LCMS showed that the starting material was consumed completely and one peak with desired mass. The reaction mixture was filtered and the filtrate was concentrated under reduced pressure to afford 3-(4-(4-(1,3-dioxolan-2-yl)piperidin-1-yl)phenyl)piperidine-2,6-dione (770 mg, crude) as a black oil. MS(M+H)⁺=345.1.

### Step 2. Synthesis of 1-(4-(2,6-dioxopiperidin-3-yl)phenyl)piperidine-4-carbaldehyde

To a mixture of 3-(4-(4-(1,3-dioxolan-2-yl)piperidin-1-yl)phenyl)piperidine-2,6-dione (770 mg, 2.24 mmol) in H₂O (7 mL) was added HCl/dioxane (4 M, 3.35 mL). The resulting mixture was stirred at 50° C. for 16 h. LCMS showed the starting material was consumed completely. The reaction mixture was concentrated under reduced pressure and the residual aqueous solution was extracted with EtOAc (10 mL×3). The combined organic layers were dried over Na₂SO₄, filtered and concentrated under reduced pressure to afford 1-(4-(2,6-dioxopiperidin-3-yl)phenyl)piperidine-4-carbaldehyde (0.4 g, 679.20 μmol, 30.38% yield, 51% purity) as a yellow solid. MS(M+H₂O+H)⁺=319.2.

### Step 3. Synthesis of tert-butyl (4-(((1-(4-(2,6-dioxopiperidin-3-yl)phenyl)piperidin-4-yl)methyl) amino)cyclohexyl)carbamate (5)

A mixture of 1-(4-(2,6-dioxopiperidin-3-yl)phenyl)piperidine-4-carbaldehyde (320 mg, 543.36 μmol), tert-butyl (4-aminocyclohexyl)carbamate (116.44 mg, 543.36 mol), 4 Å MS (170 mg) and TEA (54.98 mg, 543.36 μmol, 75.63 μL) in DCM (3 mL) was stirred at 25° C. for 0.5 h. Then NaBH(OAc)₃ (345.48 mg, 1.63 mmol) was added and the resulting mixture was stirred at 25° C. for 16 h. LCMS showed one main peak with desired mass. The reaction mixture was diluted with H₂O (20 mL) and extracted with EtOAc (5 mL×3). The combined organic layers were dried over Na₂SO₄, filtered and concentrated under reduced pressure to afford tert-butyl (4-(((1-(4-(2,6-dioxopiperidin-3-yl)phenyl)piperidin-4-yl)methyl)amino)cyclohexyl)carbamate (390 mg, crude) as a brown solid. MS(M+H)⁺=499.3.

### Step 4. Synthesis of tert-butyl (4-(((1-(4-(2,6-dioxopiperidin-3-yl)phenyl)piperidin-4-yl)methyl)(methyl)amino)cyclohexyl)carbamate (6)

A mixture of tert-butyl (4-(((1-(4-(2,6-dioxopiperidin-3-yl)phenyl)piperidin-4-yl) methyl)amino)cyclohexyl)carbamate (350 mg, 682.68 μmol), HOAc (41.00 mg, 682.68 μmol) and HCHO (277.04 mg, 3.41 mmol, 37% purity) in MeOH (4 mL) was stirred at 20° C. for 0.5 h. Then NaBH₃CN (214.50 mg, 3.41 mmol) was added and the resulting mixture was stirred at 20° C. for 16 h. LCMS showed the starting material was consumed completely and one main peak with desired mass. The reaction mixture was concentrated under reduced pressure. The residue was purified by flash silica gel chromatography (ISCO®; 12 g SepaFlash® Silica Flash Column, Eluent of 0-100% EtOAc/Petroleum ether to 0-50% MeOH/EtOAc gradient @ 100 mL/min) to afford tert-butyl (4-(((1-(4-(2,6-dioxopiperidin-3-yl)phenyl)piperidin-4-yl)methyl) (methyl)amino)cyclohexyl)carbamate (80 mg, 106.11 μmol, 15.54% yield, 68% purity) as a yellow solid. MS(M+H)⁺=513.4.

### Step 5. Synthesis of 3-(4-(4-(((4-aminocyclohexyl)(methyl)amino)methyl)piperidin-1-yl)phenyl)piperidine-2,6-dione (7)

To a solution of tert-butyl (4-(((1-(4-(2,6-dioxopiperidin-3-yl)phenyl)piperidin-4-yl)methyl)(methyl)amino)cyclo hexyl)carbamate (80 mg, 156.04 μmol) in dioxane (2 mL) was added HCl/dioxane (4 M, 2 mL). The mixture was stirred at 20° C. for 1 h. LCMS showed the starting material was consumed completely and one main peak with desired mass. The reaction mixture was concentrated under reduced pressure to afford 3-(4-(4-(((4-aminocyclohexyl)(methyl) amino)methyl)piperidin-1-yl)phenyl)piperidine-2,6-dione (70 mg, crude, HCl) as a yellow solid. MS(M+H)⁺=413.3.

### Step 6. Synthesis of N-(2-chlorophenyl)-4-((2-((4-((4-(((1-(4-(2,6-dioxopiperidin-3-yl)phenyl)piperidin-4-yl) methyl)(methyl)amino)cyclohexyl)carbamoyl)phenyl)amino)-5-fluoropyrimidin-4-yl)amino)benzamide (Compound 4)

A mixture of 4-((4-((4-((2-chlorophenyl)carbamoyl)phenyl)amino)-5-fluoropyrimidin-2-yl) amino)benzoic acid (59.60 mg, 93.54 μmol), DIPEA (36.27 mg, 280.61 μmol, 48.88 μL) and HATU (42.68 mg, 112.24 μmol) in DMF (1 mL) was stirred at 25° C. for 0.5 h. Then 3-(4-(4-(((4-aminocyclohexyl)(methyl)amino)methyl)piperidin-1-yl)phenyl)piperidine-2,6-dione (70 mg, 93.54 μmol, HCl) and DIPEA (36.27 mg, 280.61 μmol, 48.88 μL) in DMF (1 mL) was added and the resulting mixture was stirred at 25° C. for 2 h. LCMS showed one main peak with desired mass. The reaction mixture was diluted with H₂O (30 mL) and extracted with EtOAc (10 mL×3). The combined organic layers were washed with brine (15 mL), dried over Na₂SO₄, filtered and concentrated under reduced pressure to give a residue. The residue was purified by prep-HPLC (column: Phenomenex Luna C18 150*25 mm*10 um; mobile phase: [water (TFA)-ACN]; gradient: 31%-61% B over 10 min) to afford N-(2-chlorophenyl)-4-((2-((4-((4-(((1-(4-(2,6-dioxopiperidin-3-yl)phenyl)piperidin-4-yl) methyl)(methyl)amino)cyclohexyl)carbamoyl)phenyl)amino)-5-fluoropyrimidin-4-yl)amino)benzamide (20.2 mg, 13.02 μmol, 13.92% yield, 93% purity, 5TFA) as a white solid. MS(M+H)⁺=872.4.

¹H NMR (400 MHz, DMSO-d₆) δ=10.78 (s, 1H), 9.94 (s, 1H), 9.77 (s, 1H), 9.64 (s, 1H), 8.84 (br s, 1H), 8.25 (d, J=3.3 Hz, 1H), 8.09-7.97 (m, 4H), 7.86 (br d, J=6.5 Hz, 1H), 7.85-7.76 (m, 3H), 7.65 (br d, J=7.8 Hz, 1H), 7.57 (d, J=7.6 Hz, 1H), 7.45-7.38 (m, 1H), 7.33-7.27 (m, 1H), 7.09 (br d, J=8.2 Hz, 2H), 6.98 (br d, J=8.1 Hz, 2H), 4.10 (br s, 1H), 3.80-3.65 (m, 3H), 3.18-3.09 (m, 1H), 3.36-3.04 (m, 1H), 2.99-2.85 (m, 1H), 2.84-2.72 (m, 4H), 2.70-2.60 (m, 3H), 2.47-2.45 (m, 1H), 2.18-2.09 (m, 1H), 2.04-1.91 (m, 5H), 1.88-1.75 (m, 4H), 1.70-1.59 (m, 2H), 1.44-1.29 (m, 2H).

### Example 5. Synthesis of N-(2-chlorophenyl)-4-((2-((4-((1-(2-(4-(4-(2,6-dioxopiperidin-3-yl)phenyl)piperazin-1-yl)acetyl)piperidin-4-yl)carbamoyl)phenyl)amino)-5-fluoropyrimidin-4-yl)amino)benzamide (Compound 5)

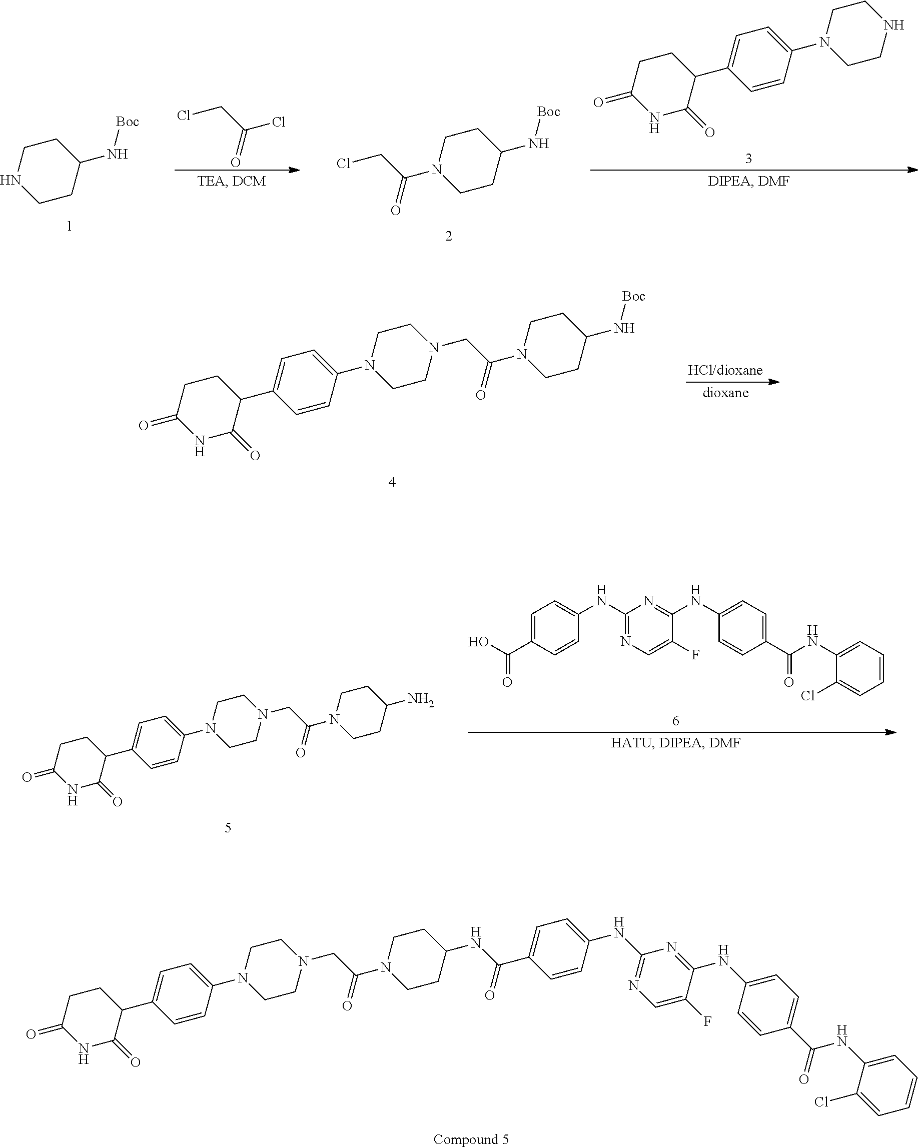

### Step 1. Synthesis of tert-butyl (1-(2-chloroacetyl)piperidin-4-yl)carbamate (2)

To a solution of tert-butyl piperidin-4-ylcarbamate (1 g, 4.99 mmol) and 2-chloroacetyl chloride (451.15 mg, 3.99 mmol) in DCM (10 mL) was added TEA (606.29 mg, 5.99 mmol). The mixture was stirred at 25° C. for 2 hr. LCMS showed the starting material was consumed completely and 73.5% peak with desired mass. The reaction mixture was concentrated under reduced pressure to afford tert-butyl (1-(2-chloroacetyl)piperidin-4-yl)carbamate (1.38 g, crude) as a yellow solid. MS(M+H)⁺=277.7.

### Step 2. Synthesis of tert-butyl (1-(2-(4-(4-(2,6-dioxopiperidin-3-yl)phenyl)piperazin-1-yl)acetyl)piperidin-4-yl)carbamate (4)

To a solution of tert-butyl (1-(2-chloroacetyl)piperidin-4-yl)carbamate (1.16 g, 4.20 mmol) and 3-(4-(piperazin-1-yl)phenyl)piperidine-2,6-dione (0.65 g, 2.10 mmol, HCl) in DMF (5 mL) was added DIPEA (813.53 mg, 6.29 mmol, 1.10 mL). The mixture was stirred at 100° C. for 24 hr. LCMS showed 66.8% peak with desired mass. The mixture was diluted with H₂O (10 mL) and extracted with EtOAc (10 mL×3). The combined organic layers were washed with brine (10 mL), dried over Na₂SO₄, filtered and concentrated under reduced pressure to give a residue. The residue was purified by flash silica gel chromatography (ISCO®; 40 g SepaFlash® Silica Flash Column, Eluent of 0-90% EtOAc/MeOH @ 100 mL/min) to afford tert-butyl (1-(2-(4-(4-(2,6-dioxopiperidin-3-yl)phenyl)piperazin-1-yl)acetyl)piperidin-4-yl)carba mate (0.971 g, 1.49 mmol, 71.09% yield, 78.9% purity) as a yellow solid. MS(M+H)⁺=514.3.

### Step 3. Synthesis of 3-(4-(4-(2-(4-aminopiperidin-1-yl)-2-oxoethyl)piperazin-1-yl)phenyl)piperidine-2,6-dione (5)

To a solution of tert-butyl (1-(2-(4-(4-(2,6-dioxopiperidin-3-yl)phenyl)piperazin-1-yl)acetyl)piperidin-4-yl)carba mate (0.971 g, 1.89 mmol) in dioxane (10 mL) was added HCl/dioxane (4 M, 10 mL). The mixture was stirred at 25° C. for 16.5 h. LCMS showed a main peak with desired mass. The reaction mixture was concentrated under reduced pressure to afford 3-(4-(4-(2-(4-aminopiperidin-1-yl)-2-oxoethyl)piperazin-1-yl)phenyl)piperidine-2,6-di one (1.1 g, crude, HCl) as a yellow solid. MS(M+H)⁺=414.3.

### Step 4. Synthesis of N-(2-chlorophenyl)-4-((2-((4-((1-(2-(4-(4-(2,6-dioxopiperidin-3-yl)phenyl)piperazin-1-yl)acetyl)piperidin-4-yl)carbamoyl)phenyl)amino)-5-fluoropyrimidin-4-yl)amino)benzamide (Compound 5)

To a solution of 3-(4-(4-(2-(4-aminopiperidin-1-yl)-2-oxoethyl)piperazin-1-yl)phenyl)piperidine-2,6-di one (207.68 mg, 461.53 μmol, HCl) and 4-((4-((4-((2-chlorophenyl)carbamoyl)phenyl)amino)-5-fluoropyrimidin-2-yl)amino)benzoic acid (0.2 g, 418.52 μmol) in DMF (2 mL) was added HATU (190.96 mg, 502.22 mol) and DIPEA (162.27 mg, 1.26 mmol, 218.70 μL). The mixture was stirred at 25° C. for 12 h. LCMS showed the starting material was consumed completely. The mixture was poured into H₂O (10 mL) and the mixture was filtered. The filter cake was collected and purified by prep-HPLC (column: Phenomenex luna C18 150×25 mm×10 um; mobile phase: [water (TFA)-ACN]; gradient: 38%-68% B over 10 min) and lyophilized to afford N-(2-chlorophenyl)-4-((2-((4-((1-(2-(4-(4-(2,6-dioxopiperidin-3-yl)phenyl)piperazin-1-yl)acetyl)piperidin-4-yl)carbamoyl)phenyl)amino)-5-fluoropyrimidin-4-yl)amino)benzamide (24.8 mg, 26.04 μmol, 6.22% yield, 91.7% purity) as a white solid. MS(M+H)⁺=873.3.

¹H NMR (400 MHz, DMSO-d₆) δ=10.79 (s, 1H), 9.93 (s, 1H), 9.76 (s, 1H), 9.63 (s, 1H), 8.25 (d, J=3.5 Hz, 1H), 8.13 (br d, J=7.5 Hz, 1H), 8.06-7.99 (m, 4H), 7.80-7.76 (m, 4H), 7.66-7.62 (m, 1H), 7.57 (dd, J=1.3, 7.9 Hz, 1H), 7.43-7.37 (m, 1H), 7.32-7.27 (m, 1H), 7.12 (d, J=8.6 Hz, 2H), 6.96 (d, J=8.8 Hz, 2H), 4.52-4.44 (m, 1H), 4.43-4.32 (m, 2H), 4.09-4.02 (m, 1H), 3.80-3.70 (m, 4H), 3.29-3.11 (m, 6H), 2.94-2.86 (m, 1H), 2.65-2.59 (m, 1H), 2.54-2.52 (m, 2H), 2.21-2.09 (m, 1H), 2.05-1.95 (m, 1H), 1.94-1.86 (m, 2H), 1.56-1.38 (m, 2H).

### Example 6. Synthesis of N-(2-chlorophenyl)-4-((2-((4-((4-(1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (Compound 6)

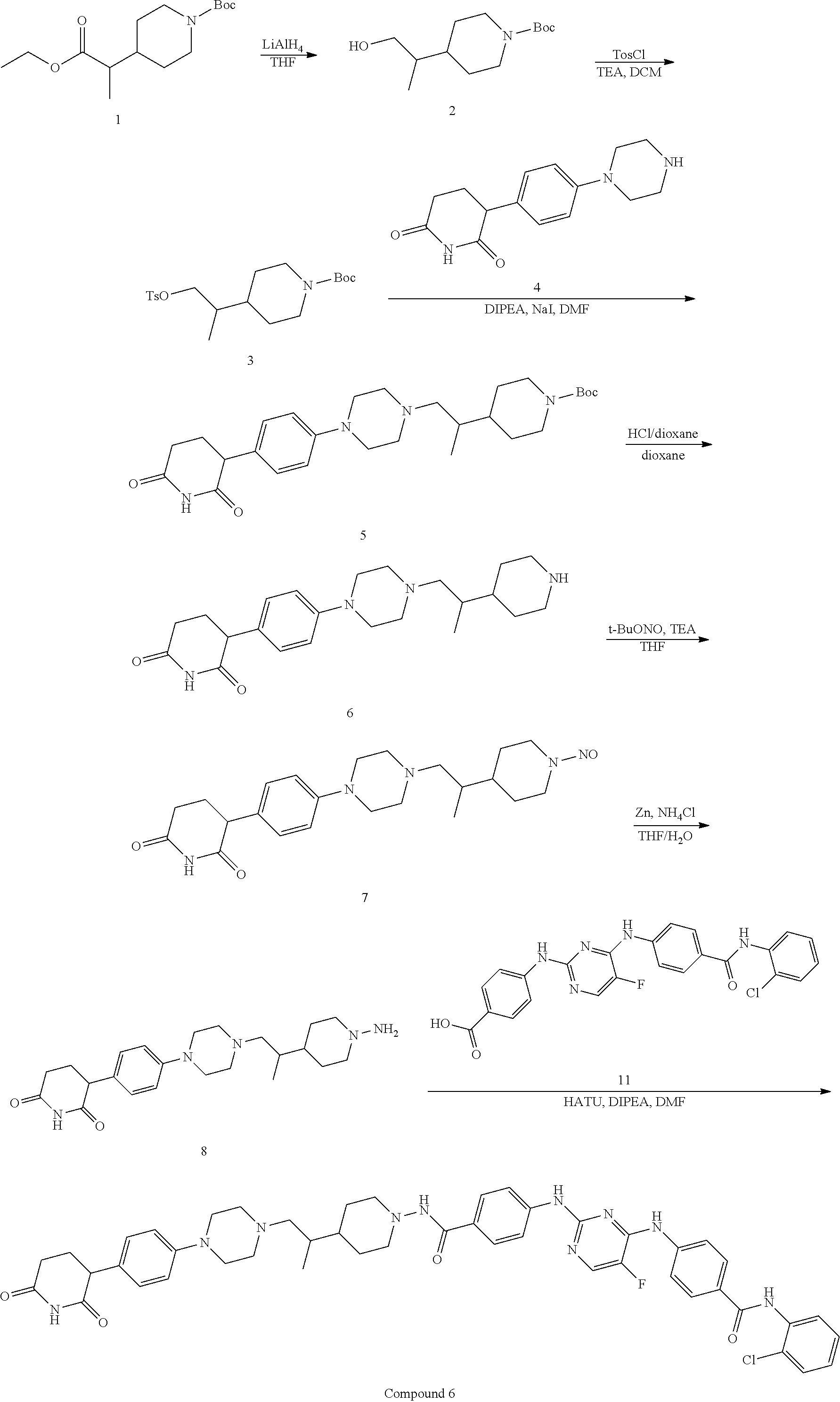

### Step 1. Synthesis of tert-butyl 4-(1-hydroxypropan-2-yl)piperidine-1-carboxylate (2)

To a solution of tert-butyl 4-(1-ethoxy-1-oxopropan-2-yl)piperidine-1-carboxylate (1 g, 3.50 mmol) in THF (20 mL) was added LiAlH₄ (2.5 M, 3 mL) dropwise at 0° C. under N₂ over 15 min. Then the mixture was stirred at 0° C. for 45 min. TLC (petroleum ether:EtOAc=2:1) showed the starting material was consumed completely and a new spot with larger polarity was formed. The reaction mixture was quenched by addition of H₂O (0.29 mL), 15% NaOH aq. (0.29 mL) and H₂O (0.86 mL) carefully. The resulting mixture was filtered and washed with EtOAc. The filtrate was dried over Na₂SO₄, filtered and concentrated under reduced pressure to afford tert-butyl 4-(1-hydroxypropan-2-yl)piperidine-1-carboxylate (0.9 g, crude) as a colorless oil. MS(M+H)⁺=244.3.

### Step 2. Synthesis of tert-butyl 4-(1-(tosyloxy)propan-2-yl)piperidine-1-carboxylate

To a solution of tert-butyl 4-(1-hydroxypropan-2-yl)piperidine-1-carboxylate (0.7 g, 2.88 mmol) and TEA (582.17 mg, 5.75 mmol, 800.78 μL) in DCM (10 mL) was added TosCl (658.11 mg, 3.45 mmol) at 20° C. The resulting mixture was stirred at 20° C. for 16 h. LCMS showed the starting material was consumed completely and one main peak with desired mass. The reaction mixture was concentrated under reduced pressure to give a residue, which was purified by flash silica gel chromatography (ISCO®; 12 g SepaFlash® Silica Flash Column, Eluent of 0-18% EtOAc/Petroleum ether, gradient @ 100 mL/min) to afford tert-butyl 4-(1-(tosyloxy)propan-2-yl)piperidine-1-carboxylate (660 mg, 1.64 mmol, 57.14% yield, 99% purity) as colorless oil. MS(M−100+H)⁺=298.1.

### Step 3. Synthesis of tert-butyl 4-(1-(4-(4-(2,6-dioxopiperidin-3-yl)phenyl)piperazin-1-yl)propan-2-yl)piperidine-1-carboxylate (5)

A mixture of tert-butyl 4-(1-(tosyloxy)propan-2-yl)piperidine-1-carboxylate (0.54 g, 1.36 mmol), 3-(4-(piperazin-1-yl)phenyl)piperidine-2,6-dione (420.82 mg, 1.36 mmol, HCl), DIPEA (526.68 mg, 4.08 mmol, 709.81 μL) and NaI (20.36 mg, 135.84 μmol) in DMF (5 mL) was stirred at 60° C. for 16 h. LCMS showed one main peak with desired mass. The reaction mixture was diluted with H₂O (20 mL) and extracted with EtOAc (5 mL×3). The combined organic layers were washed with brine (10 mL), dried over Na₂SO₄, filtered and concentrated under reduced pressure to give a residue, which was purified by flash silica gel chromatography (ISCO®; 12 g SepaFlash® Silica Flash Column, Eluent of 0-100% EtOAc/Petroleum ether, gradient @ 100 mL/min) to afford tert-butyl 4-(1-(4-(4-(2,6-dioxopiperidin-3-yl)phenyl)piperazin-1-yl)propan-2-yl)piperidine-1-carboxylate (180 mg, 324.87 μmol, 23.92% yield, 90% purity) as a light yellow solid. MS(M+H)⁺=499.4.

### Step 4. Synthesis of 3-(4-(4-(2-(piperidin-4-yl)propyl)piperazin-1-yl)phenyl)piperidine-2,6-dione (6)

To a solution of tert-butyl 4-(1-(4-(4-(2,6-dioxopiperidin-3-yl)phenyl)piperazin-1-yl)propan-2-yl)piperidine-1-carboxylate (180 mg, 360.97 μmol) in dioxane (2 mL) was added HCl/dioxane (4 M, 2 mL). The mixture was stirred at 20° C. for 1 hr. LCMS showed the starting material was consumed completely. The reaction mixture was concentrated under reduced pressure to afford 3-(4-(4-(2-(piperidin-4-yl)propyl)piperazin-1-yl)phenyl)piperidine-2,6-dione (160 mg, crude, HCl) as a yellow solid. MS(M+H)⁺=399.3.

### Step 5. Synthesis of 3-(4-(4-(2-(1-nitrosopiperidin-4-yl)propyl)piperazin-1-yl)phenyl)piperidine-2,6-dione (7)

To a solution of 3-(4-(4-(2-(piperidin-4-yl)propyl)piperazin-1-yl)phenyl)piperidine-2,6-dione (128 mg, 294.25 μmol, HCl) in THF (2 mL) was added TEA (89.33 mg, 882.76 μmol, 122.87 μL) at 0° C. Then a solution of t-BuONO (91.03 mg, 882.76 μmol, 104.99 μL) in THF (1 mL) was added drop-wise at 0° C. The resulting mixture was stirred at 60° C. for 12 h. LCMS showed the starting material was consumed completely and one main peak with desired mass. The reaction mixture was concentrated under reduced pressure to give a residue, which was purified by flash silica gel chromatography (ISCO®; 12 g SepaFlash® Silica Flash Column, Eluent of 0-100% EtOAc/Petroleum ether, gradient @ 100 mL/min) to afford 3-(4-(4-(2-(1-nitrosopiperidin-4-yl)propyl)piperazin-1-yl)phenyl)piperidine-2,6-dione (100 mg, 215.19 μmol, 73.13% yield, 92% purity) as a yellow solid. MS(M+H)⁺=428.3.

### Step 6. Synthesis of 3-(4-(4-(2-(1-aminopiperidin-4-yl)propyl)piperazin-1-yl)phenyl)piperidine-2,6-dione (8)

To a solution of 3-(4-(4-(2-(1-nitrosopiperidin-4-yl)propyl)piperazin-1-yl)phenyl)piperidine-2,6-dione (50 mg, 116.95 μmol) and NH₄Cl (25.02 mg, 467.79 μmol) in THF (2 mL) and H₂O (0.5 mL) was added Zn (30.59 mg, 467.79 μmol) at 0° C. and the mixture was stirred at 20° C. for 16 hr. LCMS showed that a main peak with desired mass. The reaction mixture was filtered and washed with THF (10 mL). The filtrate was dried over Na₂SO 4, filtered and concentrated under reduced pressure to afford 3-(4-(4-(2-(1-aminopiperidin-4-yl)propyl)piperazin-1-yl)phenyl)piperidine-2,6-dione (75 mg, crude) as a yellow solid. MS(M+H)⁺=414.3.

### Step 7. Synthesis of N-(2-chlorophenyl)-4-((2-((4-((4-(1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (Compound 6)

A mixture of 4-((4-((4-((2-chlorophenyl)carbamoyl)phenyl)amino)-5-fluoropyrimidin-2-yl)amino) benzoic acid (115.55 mg, 181.35 μmol), DIPEA (70.32 mg, 544.06 μmol, 94.77 μL) and HATU (82.75 mg, 217.63 μmol) in DMF (0.5 mL) was stirred at 25° C. for 0.5 h. Then 3-(4-(4-(2-(1-aminopiperidin-4-yl)propyl)piperazin-1-yl)phenyl)piperidine-2,6-dione (75 mg, 181.35 μmol) in DMF (0.5 mL) was added and the resulting mixture was stirred at 25° C. for 16 h. LCMS showed the starting material was consumed completely and one main peak with desired mass. The reaction mixture was diluted with H₂O (20 mL) and extracted with EtOAc (5 mL×3). The combined organic layers were washed with brine (15 mL), dried over Na₂SO₄, filtered and concentrated under reduced pressure to give a residue. The residue was purified by prep-HPLC (column: Phenomenex Luna C18 150*40 mm*15 um; mobile phase: [water (TFA)-ACN]; gradient: 20%-50% B over 10 min) and lyophilized to afford N-(2-chlorophenyl)-4-((2-((4-((4-(1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (13.3 mg, 11.71 μmol, 6.46% yield, 97% purity, 2TFA) as a white solid. MS(M+H)⁺=873.4.

¹H NMR (400 MHz, DMSO-d₆) δ=10.79 (s, 1H), 9.98 (s, 1H), 9.77 (s, 1H), 9.62 (s, 1H), 9.28-9.26 (m, 1H), 9.22-9.10 (m, 1H), 8.25 (br d, J=3.1 Hz, 1H), 8.07-7.98 (m, 4H), 7.78 (br d, J=8.9 Hz, 2H), 7.73-7.67 (m, 2H), 7.65 (br d, J=7.5 Hz, 1H), 7.57 (br d, J=8.1 Hz, 1H), 7.40 (br t, J=7.6 Hz, 1H), 7.35-7.26 (m, 1H), 7.12 (br d, J=7.7 Hz, 2H), 6.98 (br d, J=7.7 Hz, 2H), 3.86-3.68 (m, 4H), 3.28-3.18 (m, 2H), 3.12-3.02 (m, 5H), 2.81-2.60 (m, 4H), 2.24-2.09 (m, 1H), 2.08-1.88 (m, 2H), 1.69-1.56 (m, 2H), 1.52-1.37 (m, 2H), 1.37-1.18 (m, 2H), 1.04-0.94 (m, 3H).

### Example 7. Synthesis of N-(2-chlorophenyl)-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)benzamide (Compound 7)

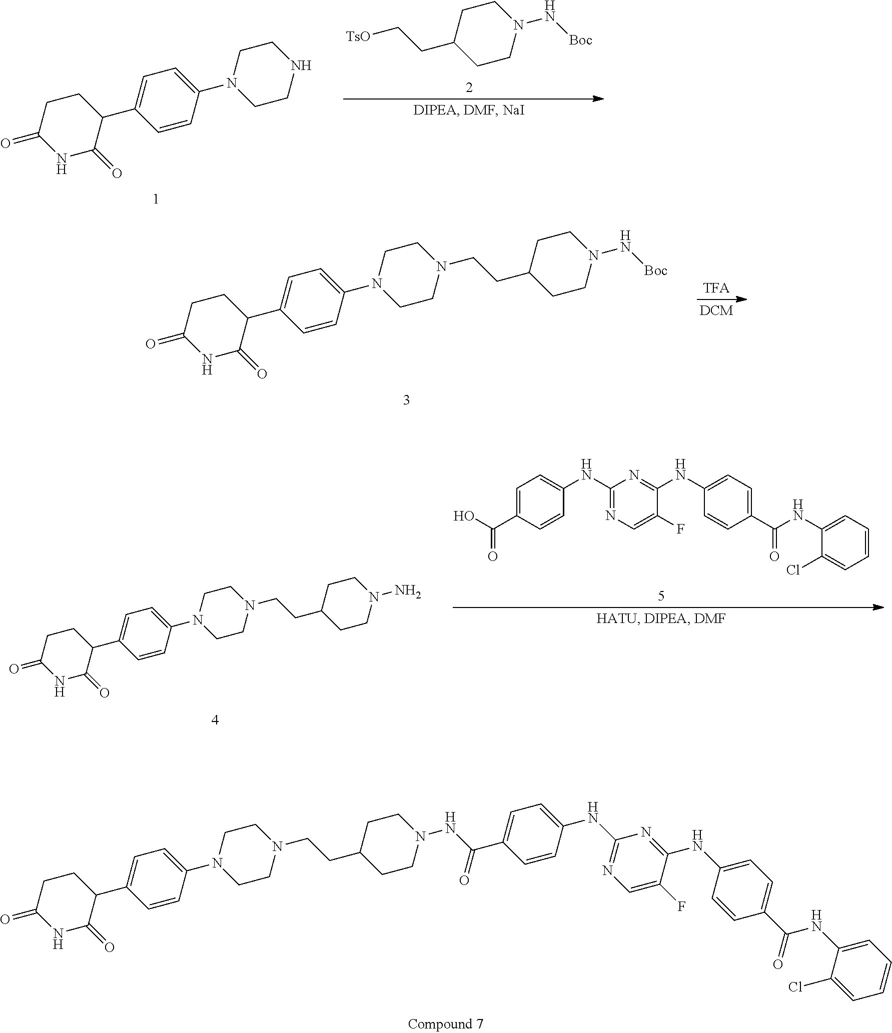 [SMILES: O=C1CCC(c2ccc(N3CCNCC3)cc2)C(=O)N1 ; Cc1ccc(S(=O)(=O)OCCC2CCN(NC(=O)OC(C)(C)C)CC2)cc1 ; CC(C)(C)OC(=O)NN1CCC(CCN2CCN(c3ccc(C4CCC(=O)NC4=O)cc3)CC2)CC1 ; O=C(O)c1ccc(Nc2ncc(F)c(Nc3ccc(C(=O)Nc4ccccc4Cl)cc3)n2)cc1 ; NN1CCC(CCN2CCN(c3ccc(C4CCC(=O)NC4=O)cc3)CC2)CC1 ; O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of tert-butyl (4-(2-(4-(4-(2,6-dioxopiperidin-3-yl)phenyl)piperazin-1-yl)ethyl)piperidin-1-yl)carbamate (2)

To a solution of 3-(4-(piperazin-1-yl)phenyl)piperidine-2,6-dione (300 mg, 1.10 mmol) and 2-(1-((tert-butoxycarbonyl)amino)piperidin-4-yl)ethyl 4-methylbenzenesulfonate (437.40 mg, 1.10 mmol) in DMF (8 mL) was added DIPEA (425.55 mg, 3.29 mmol, 573.52 μL) and NaI (32.90 mg, 219.52 μmol) at 20° C. The mixture was stirred at 60° C. for 16 h. LCMS showed 19% peak of 3-(4-(piperazin-1-yl)phenyl)piperidine-2,6-dione remained and 22% peak with desired mass. The reaction mixture was diluted with H₂O (30 mL) and extracted with EtOAc (15 mL×3). The combined organic layer was washed with brine (15 mL×3), dried over NaSO₄, filtered and concentrated in vacuum. The crude product was purified by prep-HPLC (column: Phenomenex Synergi Polar-RP 100*25 mm*4 um; mobile phase: [water (TFA)-ACN]; B %: 19%-49%, 10 min) and lyophilized to afford tert-butyl (4-(2-(4-(4-(2,6-dioxopiperidin-3-yl)phenyl)piperazin-1-yl)ethyl)piperidin-1-yl)carbamate (85 mg, 170.12 μmol, 15.50% yield, 80% purity) as a yellow oil. MS(M+H)⁺=500.4.

### Step 2. Synthesis of 3-(4-(4-(2-(1-aminopiperidin-4-yl)ethyl)piperazin-1-yl)phenyl)piperidine-2,6-dione (3)

To a solution of tert-butyl (4-(2-(4-(4-(2,6-dioxopiperidin-3-yl)phenyl)piperazin-1-yl)ethyl)piperidin-1-yl) carbamate (85 mg, 170.12 μmol) in DCM (1 mL) was added TFA (300 mg, 2.7 mmol, 0.2 mL) at 20° C. and the mixture was stirred at 20° C. for 2 hrs. LCMS showed the starting material was consumed completely and 93% peak with desired mass. The reaction mixture was concentrated under vacuum to afford 3-(4-(4-(2-(1-aminopiperidin-4-yl)ethyl)piperazin-1-yl)phenyl)piperidine-2,6-dione (125 mg, crude, TFA) as a yellow oil solid. MS(M+H)⁺=400.3.

### Step 3. Synthesis of N-(2-chlorophenyl)-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)benzamide (Compound 7)

To a solution of 4-((4-((4-((2-chlorophenyl)carbamoyl)phenyl)amino)-5-fluoropyrimidin-2-yl)amino)benzoic acid (116.32 mg, 243.40 μmol) in DMF (2 mL) were added HATU (101.80 mg, 267.74 μmol) and DIPEA (157.29 mg, 1.22 mmol, 211.98 μL) at 20° C. The mixture was stirred at 20° C. for 30 min, then a solution of 3-(4-(4-(2-(1-aminopiperidin-4-yl)ethyl)piperazin-1-yl)phenyl)piperidine-2,6-dione (125 mg, 243.40 mol TFA) in DMF (2 mL) was added and the reaction mixture was stirred at 20° C. for 12 hrs. LCMS showed starting material was consumed completely and 18% peak with desired mass. The reaction mixture was diluted with H₂O (20 mL) and extracted with EtOAc (10 mL×3). The combined organic layer was washed with brine (15 mL×3), dried over NaSO₄, filtered and concentrated in vacuum. The crude product was purified by prep-TLC (SiO₂, DCM:MeOH=10:1), followed by prep-HPLC (column: Phenomenex Luna C18 150*25 mm*10 um; mobile phase: [water (TFA)-ACN]; B %: 30%-60%, 10 min) and lyophilized to afford N-(2-chlorophenyl)-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)benzamide (7.1 mg, 5.04 μmol, 2.07% yield, 93.4% purity, 4TFA) as a white solid. MS(M+H)⁺=859.4.

¹H NMR (400 MHz, DMSO-d₆) δ=10.80 (s, 1H), 9.94 (s, 1H), 9.81-9.73 (m, 1H), 9.66 (s, 1H), 9.57-9.40 (m, 2H), 8.25 (d, J=3.5 Hz, 1H), 8.06-7.97 (m, 4H), 7.81-7.76 (m, 2H), 7.71 (d, J=8.9 Hz, 2H), 7.67-7.62 (m, 1H), 7.57 (dd, J=1.4, 8.0 Hz, 1H), 7.43-7.37 (m, 1H), 7.33-7.28 (m, 1H), 7.12 (d, J=8.5 Hz, 2H), 6.98 (br d, J=8.6 Hz, 2H), 3.84 (br d, J=12.4 Hz, 2H), 3.76 (br dd, J=4.8, 11.4 Hz, 1H), 3.63-3.60 (m, 2H), 3.30-3.20 (m, 3H), 3.20-3.11 (m, 3H), 3.00-2.92 (m, 2H), 2.85-2.75 (m, 2H), 2.70-2.65 (m, 1H), 2.56-2.55 (m, 1H), 2.21-2.09 (m, 1H), 2.05-1.96 (m, 1H), 1.78-1.69 (m, 2H), 1.68-1.60 (m, 2H), 1.43-1.30 (m, 3H).

### Example 8. Synthesis of N-(2-chlorophenyl)-4-((2-((4-((1-((1-(4-(2,6-dioxopiperidin-3-yl)phenyl)azetidin-3-yl) methyl)piperidin-4-yl)carbamoyl)phenyl)amino)-5-fluoropyrimidin-4-yl)amino)benzamide (Compound 8)

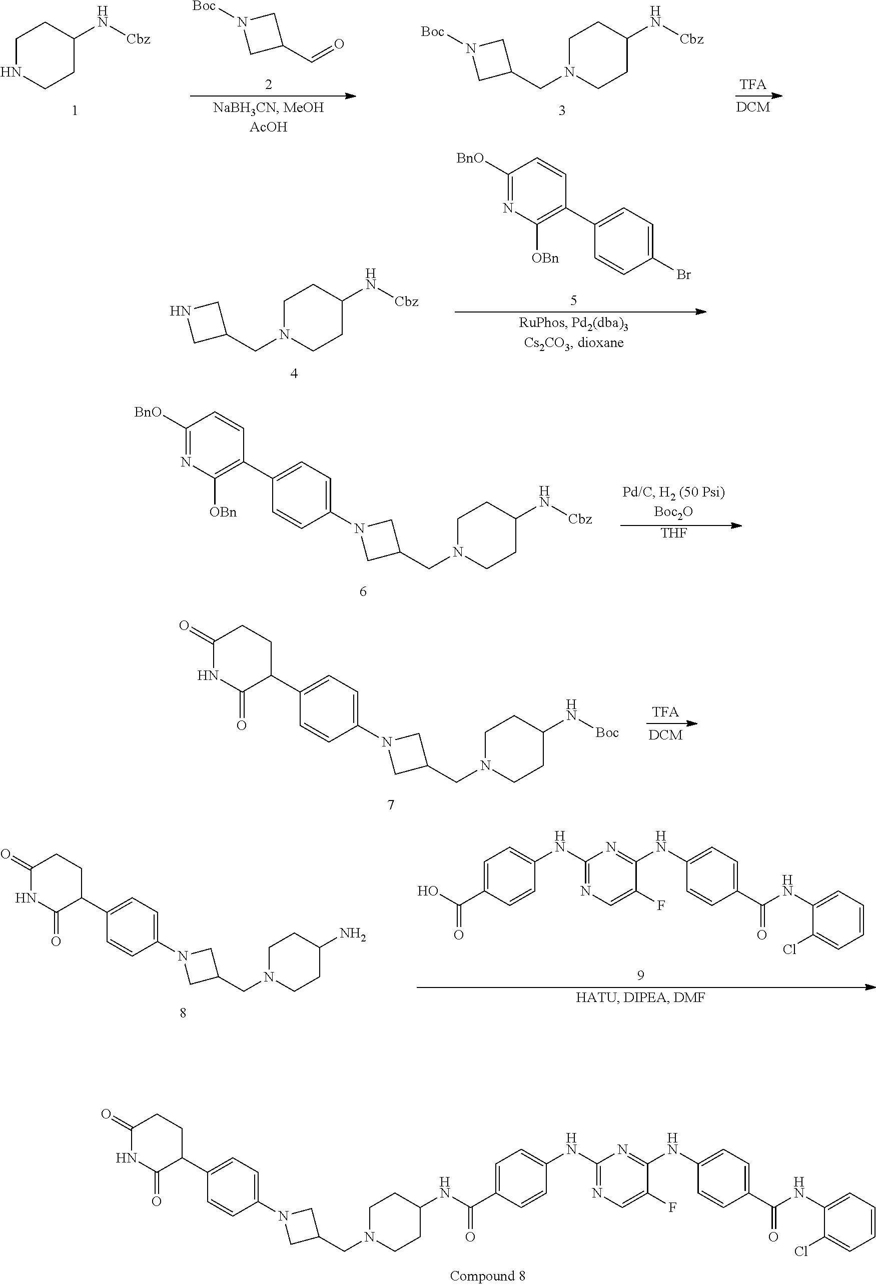

### Step 1. Synthesis of tert-butyl 3-((4-(((benzyloxy)carbonyl)amino)piperidin-1-yl)methyl) azetidine-1-carboxylate (3)

A mixture of benzyl piperidin-4-ylcarbamate (3.5 g, 14.94 mmol), tert-butyl 3-formylazetidine-1-carboxylate (3.04 g, 16.43 mmol) and HOAc (897.09 mg, 14.94 mmol) in MeOH (35 mL) was stirred at 25° C. for 0.5 h. Then NaBH₃CN (2.82 g, 44.82 mmol) was added and the resulting mixture was stirred at 25° C. for 16 h. LCMS showed that a main peak with desired mass. The reaction mixture was concentrated under reduced pressure. The residue was diluted with NaHCO₃ solution (100 mL) and extracted with EtOAc (30 mL×3). The combined organic layers were dried over Na₂SO₄, filtered and concentrated in vacuum. The residue was purified by flash silica gel chromatography (80 g SepaFlash® Silica Flash Column, eluent of 30-100% EtOAc/petroleum ether, gradient @ 200 mL/min) to afford tert-butyl 3-((4-(((benzyloxy)carbonyl)amino)piperidin-1-yl)methyl)azetidine-1-carboxylate (9 g) as a colorless oil. MS(M+H)⁺=404.2.

### Step 2. Synthesis of benzyl (1-(azetidin-3-ylmethyl)piperidin-4-yl)carbamate (4)

To a solution of tert-butyl 3-((4-(((benzyloxy)carbonyl)amino)piperidin-1-yl)methyl)azetidine-1-carboxylate (1 g, 2.48 mmol) in DCM (10 mL) was added TFA (3.07 g, 26.92 mmol, 2.00 mL). The mixture was stirred at 20° C. for 1 h. LCMS showed the starting material was consumed completely and one main peak with desired mass. The reaction mixture was concentrated in vacuum to afford benzyl (1-(azetidin-3-ylmethyl)piperidin-4-yl)carbamate (1 g, TFA salt) as a colorless oil, which was used directly. MS(M+H)⁺=304.2.

### Step 3. Synthesis of benzyl (1-((1-(4-(2,6-bis(benzyloxy)pyridin-3-yl)phenyl)azetidin-3-yl)methyl)piperidin-4-yl)carbamate (6)

To a solution of benzyl (1-(azetidin-3-ylmethyl)piperidin-4-yl)carbamate (600.00 mg, 1.44 mmol, TFA salt) and 2,6-bis(benzyloxy)-3-(4-bromophenyl)pyridine (534.63 mg, 1.20 mmol) in dioxane (20 mL) were added Cs₂CO₃ (1.17 g, 3.59 mmol), RuPhos (33.54 mg, 71.87 μmol) and Pd₂(dba)₃ (54.84 mg, 59.89 μmol) under nitrogen. The mixture was stirred at 100° C. for 16 hr. LCMS showed benzyl (1-(azetidin-3-ylmethyl)piperidin-4-yl)carbamate was consumed completely and one main peak with desired mass. The reaction mixture was filtered and washed with EtOAc. The filtrate was concentrated in vacuum. The residue was purified by flash silica gel chromatography (20 g SepaFlash® Silica Flash Column, eluent of 50-100% EtOAc/petroleum ether to 0~20% MeOH/EtOAc, gradient @ 100 mL/min) to afford benzyl (1-((1-(4-(2,6-bis(benzyloxy)pyridin-3-yl)phenyl)azetidin-3-yl)methyl)piperidin-4-yl)carbamate (680 mg, 904.88 μmol, 75.54% yield, 89% purity) as a yellow solid. MS(M+H)⁺=669.4.

### Step 4. Synthesis of tert-butyl (1-((1-(4-(2,6-dioxopiperidin-3-yl)phenyl)azetidin-3-yl)methyl)piperidin-4-yl)carbamate (7)

To a solution of benzyl (1-((1-(4-(2,6-bis(benzyloxy)pyridin-3-yl)phenyl)azetidin-3-yl)methyl)piperidin-4-yl)carbamate (340 mg, 508.36 μmol) in THF (10 mL) were added Boc₂O (332.84 mg, 1.53 mmol, 350.36 μL) and Pd/C (200 mg, 10% purity) under N₂ atmosphere, the mixture was degassed under vacuum and purged with H₂ several times. The resulting mixture was stirred at 30° C. for 40 hours under H₂ (50 psi) atmosphere. LCMS showed 25% of the intermediate remained and 30% of desired mass was detected. The reaction mixture was filtered and washed with THF (200 mL). The filtrate was concentrated under reduced pressure to give a residue. To a solution of the residue in THF (20 mL) was added Pd/C (0.4 g, 10% purity) under N₂ atmosphere, the suspension was degassed under vacuum and purged with H₂ several times. The mixture was stirred at 30° C. for 16 hours under H₂ (50 psi) atmosphere. LCMS showed the intermediate was consumed completely and 35% of desired mass was detected. The reaction mixture was filtered and washed with THF (200 mL). The filtrate was concentrated under reduced pressure to give a residue, which was purified by prep-TLC (SiO₂, DCM:MeOH=10:1) to afford tert-butyl (1-((1-(4-(2,6-dioxopiperidin-3-yl)phenyl)azetidin-3-yl) methyl)piperidin-4-yl)carbamate (120 mg, 262.83 μmol, 20.00% yield) as a white solid. MS(M+H)⁺=457.2.

### Step 5. Synthesis of 3-(4-(3-((4-aminopiperidin-1-yl)methyl)azetidin-1-yl)phenyl)piperidine-2,6-dione (8)

A mixture of tert-butyl (1-((1-(4-(2,6-dioxopiperidin-3-yl)phenyl)azetidin-3-yl)methyl)piperidin-4-yl)carbamate (120 mg, 262.83 μmol) and TFA (1.54 g, 13.46 mmol, 1 mL) in DCM (4 mL) was stirred at 15° C. for 1 hour. LCMS showed the starting material was consumed completely. The mixture was concentrated in vacuum to afford 3-(4-(3-((4-aminopiperidin-1-yl)methyl) azetidin-1-yl)phenyl)piperidine-2,6-dione (160 mg, 2TFA salt) as a colorless gum, which was used directly. MS(M+H)⁺=357.2.

### Step 6. Synthesis of N-(2-chlorophenyl)-4-((2-((4-((1-((1-(4-(2,6-dioxopiperidin-3-yl)phenyl)azetidin-3-yl)methyl)piperidin-4-yl)carbamoyl)phenyl)amino)-5-fluoropyrimidin-4-yl)amino)benzamide (Compound 8)

A mixture of 4-((4-((4-((2-chlorophenyl)carbamoyl)phenyl)amino)-5-fluoropyrimidin-2-yl) amino)benzoic acid (130 mg, 272.04 μmol), HATU (134.47 mg, 353.65 μmol) and DIPEA (351.59 mg, 2.72 mmol, 473.84 μL) in DMF (2 mL) was stirred at 15° C. for 15 minutes, then 3-(4-(3-((4-aminopiperidin-1-yl)methyl)azetidin-1-yl)phenyl)piperidine-2,6-dione (160 mg, 273.73 μmol, 2TFA salt) was added. The resulting mixture was stirred at 15° C. for 1 hour. LCMS showed 3-(4-(3-((4-aminopiperidin-1-yl)methyl)azetidin-1-yl)phenyl)piperidine-2,6-dione was consumed completely and 56% peak with the desired mass. The mixture was treated with TFA to adjust pH<7 and the resulting mixture was purified by prep-HPLC (column: Phenomenex luna C18 150*40 mm*15 um; mobile phase: [water(TFA)-ACN]; gradient: 15%-45% B over 10 min) and lyophilized to afford the crude product. F NMR and H NMR indicated the product was not clean. The residue was triturated with a mixture (H₂O:ACN:DCM:MeOH=4:1:1:1, 21 mL) for 10 minutes and filtered. The filter cake was collected and dried. The crude product was triturated with a mixture (H₂O:THF:ACN:DCM:MeOH=1:1:2:3:1, 16 mL) for 10 minutes and lyophilized to afford N-(2-chlorophenyl)-4-((2-((4-((1-((1-(4-(2,6-dioxopiperidin-3-yl)phenyl)azetidin-3-yl)methyl)piperidin-4-yl)carbamoyl)phenyl)amino)-5-fluoropyrimidin-4-yl)amino)benzamide (77.3 mg, 85.51 μmol, 31.43% yield, 90.3% purity) as a gray solid. MS(M+H)⁺=816.3.

¹H NMR (400 MHz, DMSO-d₆) δ=10.99-10.55 (m, 1H), 10.15-9.87 (m, 1H), 9.83-9.69 (m, 1H), 9.68-9.41 (m, 1H), 8.32-8.19 (m, 1H), 8.17-7.91 (m, 5H), 7.91-7.71 (m, 4H), 7.69-7.51 (m, 2H), 7.48-7.22 (m, 2H), 7.17-6.84 (m, 2H), 6.63-6.18 (m, 2H), 4.12-3.81 (m, 2H), 3.80-3.61 (m, 2H), 3.50-3.42 (m, 2H), 3.05-2.72 (m, 4H), 2.41-2.29 (m, 2H), 2.25-1.89 (m, 5H), 1.80-1.70 (m, 2H), 1.66-1.44 (m, 2H).

### Example 9. Synthesis of N-(2-chlorophenyl)-4-((2-((4-((1-((1-(4-(2,6-dioxopiperidin-3-yl)phenyl)pyrrolidin-3-yl)methyl)piperidin-4-yl)carbamoyl)phenyl)amino)-5-fluoropyrimidin-4-yl)amino)benzamide (Compound 9)

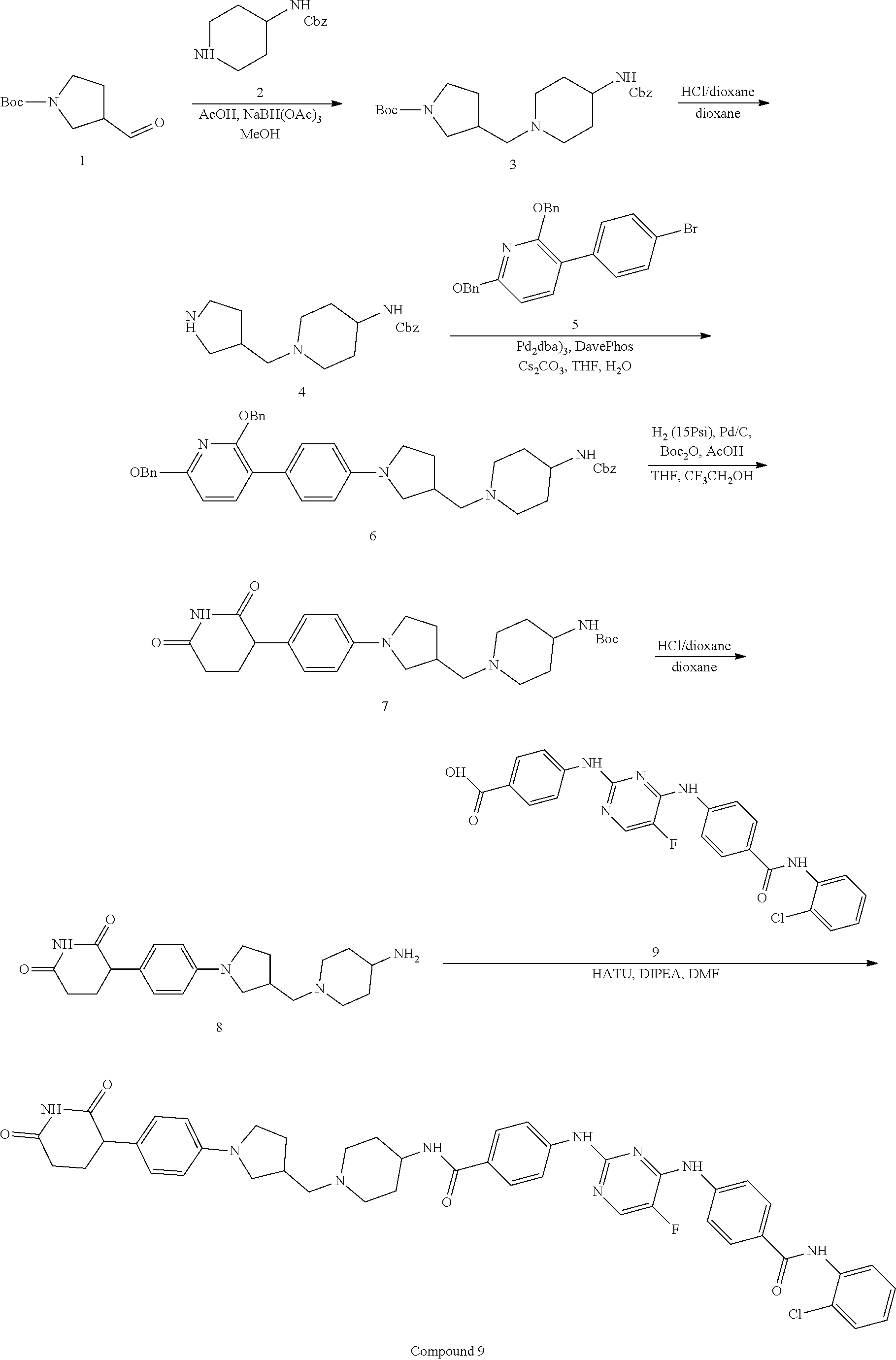

### Step 1. Synthesis of tert-butyl 3-((4-(((benzyloxy)carbonyl)amino)piperidin-1-yl)methyl)pyrrolidine-1-carboxylate

To a solution of benzyl N-(4-piperidyl) carbamate (1 g, 4.27 mmol) and tert-butyl 3-formylpyrrolidine-1-carboxylate (1.02 g, 5.12 mmol) in MeOH (30 mL) was added AcOH (256.31 mg, 4.27 mmol, 244.34 μL) and NaBH(OAc)₃ (1.81 g, 8.54 mmol). The mixture was stirred at 25° C. for 14 hr. LCMS showed 71% peak with desired mass. The reaction mixture was concentrated under reduced pressure to remove the organic solvent. The residue was diluted with H₂O (30 mL) and extracted with EtOAc (10 mL×3). The combined organic layers were washed with brine (20 mL), dried over Na₂SO₄, filtered and concentrated under reduced pressure to give a residue. The residue was purified by flash silica gel chromatography (ISCO®; 40 g SepaFlash® Silica Flash Column, Eluent of 0-10% MeOH/DCM @ 85 mL/min) to afford tert-butyl 3-((4-(((benzyloxy)carbonyl)amino)piperidin-1-yl)methyl)pyrrolidine-1-carboxylate (1.7 g, 3.79 mmol, 88.71% yield, 93% purity) as a white solid. MS(M+H)⁺=418.3.

### Step 2. Synthesis of benzyl (1-(pyrrolidin-3-ylmethyl)piperidin-4-yl)carbamate (4)

To a solution of tert-butyl tert-butyl 3-((4-(((benzyloxy)carbonyl)amino)piperidin-1-yl)methyl)pyrrolidine-1-carboxylate (1.7 g, 4.07 mmol) in dioxane (10 mL) was added HCl/dioxane (4 M, 10 mL). The mixture was stirred at 25° C. for 6 hr. LCMS showed the starting material was consumed completely. The reaction mixture was concentrated under reduced pressure to afford benzyl N-[1-(pyrrolidin-3-ylmethyl)-4-piperidyl]carbamate (1.45 g, crude, HCl) as a yellow solid. MS(M+H)⁺=318.2.

### Step 3. Synthesis of benzyl (1-((1-(4-(2,6-bis(benzyloxy)pyridin-3-yl)phenyl)pyrrolidin-3-yl)methyl)piperidin-4-yl)carbamate (6)

To a solution of benzyl (1-(pyrrolidin-3-ylmethyl)piperidin-4-yl)carbamate (675 mg, 2.13 mmol) and 2,6-bis(benzyloxy)-3-(4-bromophenyl)pyridine (632.75 mg, 1.42 mmol) in THF (30 mL) and H₂O (3 mL) were added Pd₂ (dba)₃ (129.82 mg, 141.77 mol), Cs₂CO₃ (923.80 mg, 2.84 mmol) and DavePhos (55.79 mg, 141.77 μmol). The mixture was stirred at 100° C. for 14 h under N₂. LCMS showed 27% peak with desired mass. The residue was diluted with H₂O (30 mL) and extracted with EtOAc (10 mL×3). The combined organic layers were washed with brine (10 mL), dried over Na₂SO₄, filtered and concentrated under reduced pressure to give a residue. The residue was purified by flash silica gel chromatography (ISCO®; 40 g SepaFlash® Silica Flash Column, Eluent of 0-100% EtOAc/petroleum ether gradient @ 100 mL/min) to afford benzyl (1-((1-(4-(2,6-bis(benzyloxy)pyridin-3-yl)phenyl)pyrrolidin-3-yl)methyl)piperidin-4-yl)carbamate (583 mg, 665.95 μmol, 23.49% yield, 78% purity) as a yellow solid. MS(M+H)⁺=683.4.

### Step 4. Synthesis of tert-butyl (1-((1-(4-(2,6-dioxopiperidin-3-yl)phenyl)pyrrolidin-3-yl)methyl)piperidin-4-yl)carbamate (7)

To a solution of benzyl (1-((1-(4-(2,6-bis(benzyloxy)pyridin-3-yl)phenyl)pyrrolidin-3-yl)methyl)piperidin-4-yl)carbamate (533 mg, 780.55 μmol) in THF (3 mL) and CF₃CH₂OH (5 mL) were added Boc₂O (340.71 mg, 1.56 mmol, 358.64 μL), Pd/C (83.07 mg, 78.06 μmol, 10% purity) and AcOH (46.87 mg, 780.55 μmol, 44.68 μL). The resulting mixture was stirred at 25° C. under H₂ (15 psi) for 72 h. LCMS showed the starting material was consumed completely. The reaction mixture was filtered and the filtrate was concentrated under reduced pressure to give a residue. The residue was purified by flash silica gel chromatography (ISCO®; 12 g SepaFlash® Silica Flash Column, Eluent of 0-100% EtOAc/petroleum ether, gradient @ 45 mL/min) to afford tert-butyl (1-((1-(4-(2,6-dioxopiperidin-3-yl)phenyl)pyrrolidin-3-yl)methyl)piperidin-4-yl) carbamate (56 mg, 97.58 μmol, 12.50% yield, 82% purity) as a white solid. MS(M+H)⁺=471.2.

### Step 5. Synthesis of 3-(4-(3-((4-aminopiperidin-1-yl)methyl)pyrrolidin-1-yl)phenyl)piperidine-2,6-dione

To a solution of tert-butyl N-[1-[[1-[4-(2,6-dioxo-3-piperidyl)phenyl]pyrrolidin-3-yl]methyl]-4-piperidyl]carbamate (56 mg, 119.00 μmol) in dioxane (2 mL) was added HCl/dioxane (4 M, 2 mL). The mixture was stirred at 25° C. for 12 hr. LCMS showed 36% peak with desired mass. The reaction mixture was concentrated under reduced pressure to afford 3-(4-(3-((4-aminopiperidin-1-yl)methyl)pyrrolidin-1-yl)phenyl)piperidine-2,6-dione (103 mg, crude, HCl) as a yellow solid. MS(M+H)⁺=371.2.

### Step 6. Synthesis of N-(2-chlorophenyl)-4-((2-((4-((1-((1-(4-(2,6-dioxopiperidin-3-yl)phenyl)pyrrolidin-3-yl) methyl)piperidin-4-yl)carbamoyl)phenyl)amino)-5-fluoropyrimidin-4-yl)amino)benzamide (Compound 9)

To a solution of 3-(4-(3-((4-aminopiperidin-1-yl)methyl)pyrrolidin-1-yl)phenyl)piperidine-2,6-dione (40 mg, 98.29 μmol, HCl) and 4-((4-((4-((2-chlorophenyl)carbamoyl)phenyl)amino)-5-fluoropyrimidin-2-yl)amino)benzoic acid (40.00 mg, 83.70 μmol) in DMF (0.5 mL) were added HATU (44.85 mg, 117.95 μmol) and DIPEA (38.11 mg, 294.88 μmol). The resulting mixture was stirred at 20° C. for 1 h. LCMS showed a peak (44%) with desired mass. The reaction mixture was filtered. The filtrate was purified by prep-HPLC (column: Phenomenex luna C18 150*25 mm*10 um; mobile phase: [water (FA)-ACN]; gradient: 22%-52% B over 10 min) and lyophilized to afford N-(2-chlorophenyl)-4-((2-((4-((1-((1-(4-(2,6-dioxopiperidin-3-yl)phenyl)pyrrolidin-3-yl) methyl)piperidin-4-yl)carbamoyl)phenyl)amino)-5-fluoropyrimidin-4-yl)amino)benzamide (10 mg, 12.04 μmol, 12.25% yield, 90% purity) as a white solid. MS(M+H)⁺=830.2.

¹H NMR (400 MHz, DMSO-d₆) δ=10.74 (s, 1H), 9.93 (s, 1H), 9.74 (s, 1H), 9.60 (s, 1H), 8.29 (s, 1H), 8.25 (d, J=3.4 Hz, 1H), 8.05-7.98 (m, 5H), 7.79-7.76 (m, 3H), 7.68-7.63 (m, 1H), 7.56 (dd, J=1.3, 8.0 Hz, 1H), 7.43-7.36 (m, 1H), 7.32-7.25 (m, 1H), 6.99 (d, J=8.6 Hz, 2H), 6.47 (d, J=8.7 Hz, 2H), 3.77-3.74 (m, 1H), 3.70-3.65 (m, 1H), 3.25-3.20 (m, 2H), 2.97-2.85 (m, 3H), 2.65-2.57 (m, 1H), 2.46-2.40 (m, 1H), 2.39-2.27 (m, 3H), 2.18-1.93 (m, 6H), 1.80-1.68 (m, 3H), 1.64-1.54 (m, 2H).

### Example 10. Synthesis of N-(2-chlorophenyl)-4-((2-((4-(((1S,2R)-2-(((1-(4-(2,6-dioxopiperidin-3-yl)phenyl)piperidin-4-yl)methyl)(methyl)amino)cyclopentyl)carbamoyl)phenyl)amino)-5-fluoropyrimidin-4-yl)amino)benzamide (Compound 10)

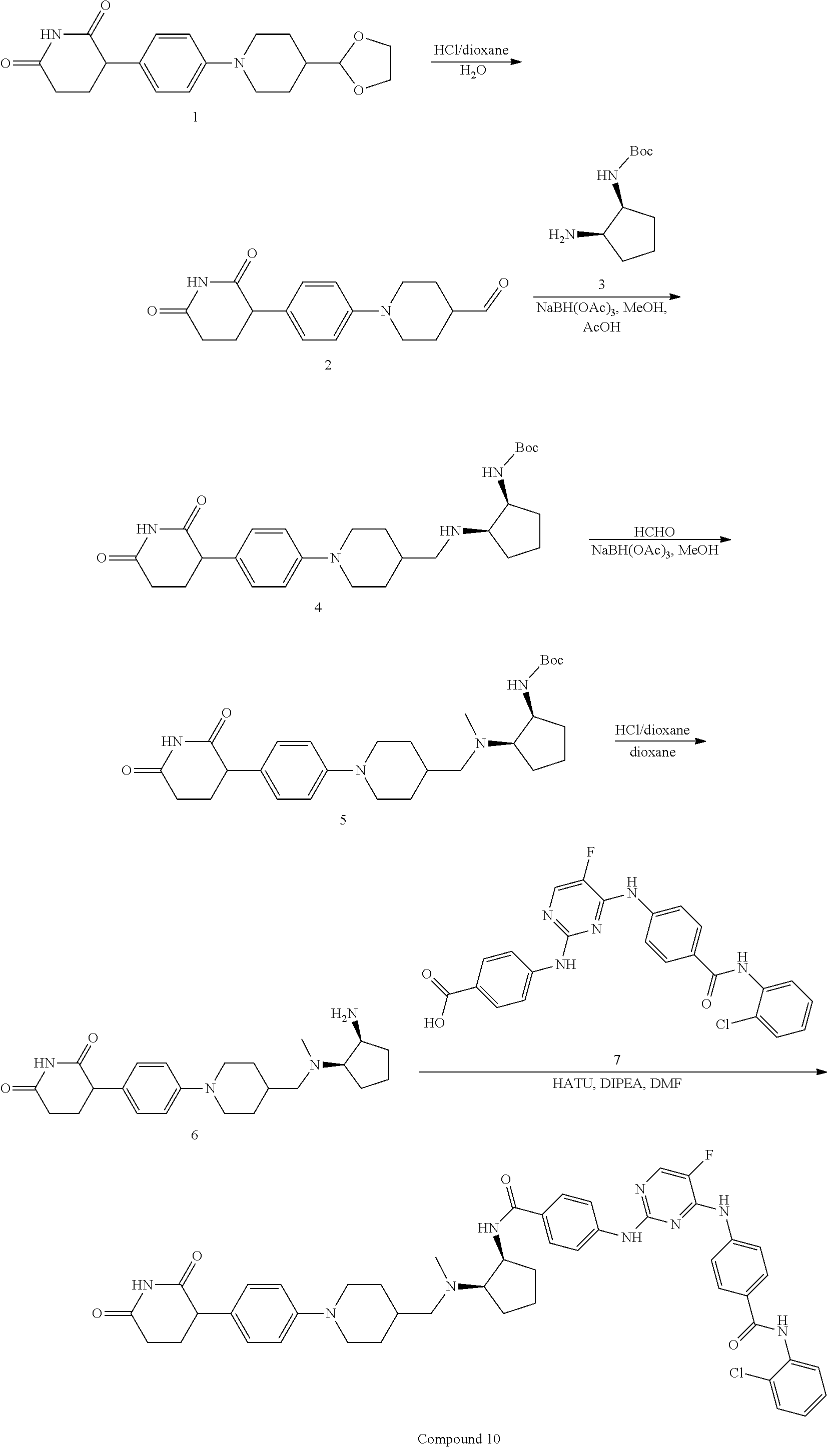

### Step 1. Synthesis of 1-(4-(2,6-dioxopiperidin-3-yl)phenyl)piperidine-4-carbaldehyde (2)

To a solution of 3-(4-(4-(1,3-dioxolan-2-yl)piperidin-1-yl)phenyl)piperidine-2,6-dione (390 mg, 1.13 mmol) in H₂O (5 mL) was added HCl/dioxane (4 M, 1.70 mL). The mixture was stirred at 50° C. for 16 hr. LCMS showed the starting material was consumed completely and 76% peak with desired mass. The mixture was diluted with NaHCO₃ (20 mL) and extracted with solvent EtOAc (10 mL×3). The combined organic layers were washed with brine (10 mL), dried over Na₂SO₄, filtered and concentrated under reduced pressure to afford 1-(4-(2,6-dioxopiperidin-3-yl)phenyl)piperidine-4-carbaldehyde (242 mg, 729.98 mol, 64.46% yield, 90.6% purity) as a brown solid. MS(M+H)⁺=301.4.

### Step 2. Synthesis of tert-butyl ((1S,2R)-2-(((1-(4-(2,6-dioxopiperidin-3-yl)phenyl)piperidin-4-yl) methyl)amino)cyclopentyl)carbamate (4)

To a solution of 1-(4-(2,6-dioxopiperidin-3-yl)phenyl)piperidine-4-carbaldehyde (220 mg, 732.48 μmol) and tert-butyl ((1S,2R)-2-aminocyclopentyl)carbamate (146.70 mg, 732.48 μmol) in MeOH (5 mL) were added NaBH(OAc)₃ (155.24 mg, 732.48 mol) and AcOH (87.97 mg, 1.46 mmol, 83.86 μL). The mixture was stirred at 25° C. for 38 h. LCMS showed the starting material was consumed completely and 39.6% peak with desired mass. The reaction mixture was concentrated under reduced pressure to remove MeOH. The residue was diluted with H₂O (20 mL) and extracted with EtOAc (10 mL×3). The combined organic layers were washed with brine (10 mL), dried over Na₂SO₄, filtered and concentrated under reduced pressure to give a residue, which was purified by flash silica gel chromatography (ISCO®; 40 g SepaFlash® Silica Flash Column, Eluent of 0-100% EtOAc/Petroleum ether, gradient @ 100 mL/min) to afford tert-butyl ((1S,2R)-2-(((1-(4-(2,6-dioxopiperidin-3-yl)phenyl)piperidin-4-yl)methyl)amino)cyclo pentyl)carbamate (170 mg, 308.34 μmol, 42.10% yield, 87.9% purity) as a white solid. MS(M+H)⁺=485.3.

### Step 3. Synthesis of tert-butyl ((1S,2R)-2-(((1-(4-(2,6-dioxopiperidin-3-yl)phenyl)piperidin-4-yl)methyl)(methyl)amino)cyclopentyl)carbamate (5)

To a solution of tert-butyl ((1S,2R)-2-(((1-(4-(2,6-dioxopiperidin-3-yl)phenyl)piperidin-4-yl)methyl)amino)cyclo pentyl)carbamate (150 mg, 309.51 μmol) in MeOH (3 mL) was added HCHO (37.68 mg, 464.27 μmol, 37% purity) and NaBH(OAc)₃ (131.20 mg, 619.03 μmol). The mixture was stirred at 20° C. for 18 h. LC-MS showed 40% peak with desired mass. The reaction mixture was concentrated under reduced pressure to remove MeOH. The residue was diluted with H₂O (20 mL) and extracted with EtOAc (10 mL×3). The combined organic layers were washed with brine (10 mL), dried over Na₂SO₄, filtered and concentrated under reduced pressure to give a residue, which was purified by flash silica gel chromatography (ISCO®; 4 g SepaFlash® Silica Flash Column, Eluent of 0-100% EtOAc/Petroleum ether, gradient @ 45 mL/min) to afford tert-butyl ((1S,2R)-2-(((1-(4-(2,6-dioxopiperidin-3-yl)phenyl)piperidin-4-yl)methyl)(methyl)amino)cyclopentyl)carbamate (100 mg, 200.54 μmol, 64.79% yield) as a white solid. MS(M+H)⁺=499.3.

### Step 4. Synthesis of 3-(4-(4-((((1R,2S)-2-aminocyclopentyl)(methyl)amino)methyl)piperidin-1-yl)phenyl)piperidine-2,6-dione (6)

To a solution of tert-butyl ((1S,2R)-2-(((1-(4-(2,6-dioxopiperidin-3-yl)phenyl)piperidin-4-yl)methyl)(methyl)amino)cyclopentyl)carbamate (100 mg, 200.54 μmol) in dioxane (5 mL) was added HCl (4 M, 150.40 μL). The mixture was stirred at 20° C. for 14 hr. LCMS showed the starting material was consumed completely. The reaction mixture was concentrated under reduced pressure to afford 3-(4-(4-((((1R,2S)-2-aminocyclopentyl)(methyl)amino)methyl)piperidin-1-yl)phenyl)piperidine-2,6-dione (160 mg, crude, HCl) as a white solid. MS(M+H)⁺=399.2.

### Step 5. Synthesis of N-(2-chlorophenyl)-4-((2-((4-(((1S,2R)-2-(((1-(4-(2,6-dioxopiperidin-3-yl)phenyl)piperidin-4-yl)methyl)(methyl)amino)cyclopentyl)carbamoyl)phenyl)amino)-5-fluoropyrimidin-4-yl)amino)benzamide (Compound 10)

To a solution of 3-(4-(4-((((1R,2S)-2-aminocyclopentyl)(methyl)amino)methyl)piperidin-1-yl)phenyl)piperidine-2,6-dione (140 mg, 321.84 μmol, HCl) and 4-((4-((4-((2-chlorophenyl) carbamoyl)phenyl)amino)-5-fluoropyrimidin-2-yl)amino)benzoic acid (153.80 mg, 321.84 μmol) in DMF (3 mL) was added HATU (146.85 mg, 386.21 μmol) and DIPEA (124.79 mg, 965.51 μmol, 168.18 μL). The mixture was stirred at 25° C. for 6 hr. LCMS showed the starting material was consumed completely and 29% peak with desired mass. The reaction mixture was diluted with H₂O (15 mL) and extracted with EtOAc (5 mL×3). The combined organic layers were washed with brine (15 mL), dried over Na₂SO₄, filtered and concentrated under reduced pressure to give a residue. The residue was purified by prep-TLC (SiO₂, DCM:MeOH=10:1), followed by prep-HPLC (column: Phenomenex luna C18 150×25 mm×10 um; mobile phase: [water (FA)-ACN]; gradient: 17%-47% B over 10 min) and lyophilized to afford N-(2-chlorophenyl)-4-((2-((4-(((1S,2R)-2-(((1-(4-(2,6-dioxopiperidin-3-yl)phenyl)piperid in-4-yl)methyl)(methyl)amino)cyclopentyl)carbamoyl)phenyl)amino)-5-fluoropyrimidin-4-yl)amino)benzamide (10.2 mg, 11.53 μmol, 3.58% yield, 97% purity) as a white solid. MS(M+H)⁺=858.4.

¹H NMR (400 MHz, DMSO-d₆) δ=10.73 (s, 1H), 9.90 (s, 1H), 9.74 (s, 1H), 9.64 (s, 1H), 8.24 (d, J=3.5 Hz, 1H), 8.10-8.00 (m, 4H), 7.82 (d, J=8.8 Hz, 2H), 7.70 (d, J=8.8 Hz, 2H), 7.67-7.63 (m, 1H), 7.59-7.53 (m, 1H), 7.43-7.39 (m, 1H), 7.38-7.34 (m, 1H), 7.32-7.26 (m, 1H), 6.93 (d, J=8.3 Hz, 2H), 6.68 (br d, J=8.8 Hz, 2H), 4.19-4.11 (m, 1H), 3.69-3.62 (m, 1H), 3.59-3.47 (m, 2H), 2.69-2.54 (m, 6H), 2.19-2.13 (m, 4H), 2.00-1.80 (m, 5H), 1.75-1.55 (m, 6H), 1.15-1.02 (m, 1H), 0.99-0.83 (m, 1H).

### Example 11. Synthesis of N-(2-chlorophenyl)-4-((2-((4-((3-(((1-(4-(2,6-dioxopiperidin-3-yl)phenyl)piperidin-4-yl)methyl)(methyl)amino)cyclobutyl)carbamoyl)phenyl)amino)-5-fluoropyrimidin-4-yl)amino)benzamide (Compound 11)

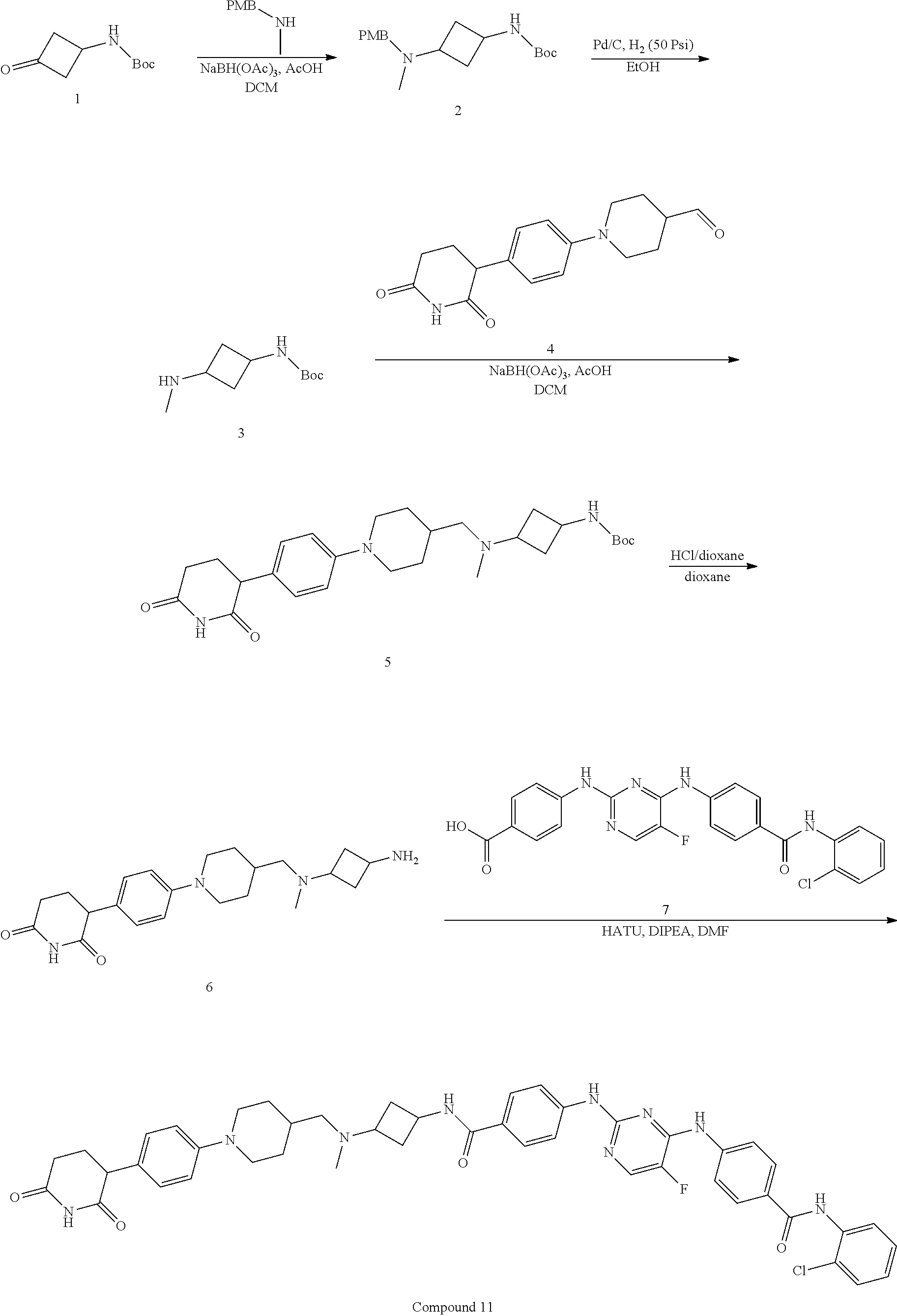

### Step 1. Synthesis of tert-butyl (3-((4-methoxybenzyl(methyl)amino)cyclobutyl)carbamate (2)

To a solution of tert-butyl (3-oxocyclobutyl) carbamate (2 g, 10.80 mmol) and 1-(4-methoxyphenyl)-N-methylmethananmine (1.63 g, 10.80 mmol) in DCM (40 mL) was added AcOH (684.44 mg, 10.80 mmol, 618.15 μL) at 20° C. The mixture was stirred at 20° C. for 30 min, then NaBH(OAc)₃ (2.29 g, 10.80 mmol) was slowly added and the resulting mixture was stirred at 20° C. for 12 h. LCMS showed the starting material was consumed completely and 87% peak with desired mass. The reaction mixture was filtered. The filtrate was diluted with H₂O (80 mL) and extracted with EtOAc (40 mL×3). The combined organic layer was washed with NaHCO₃ (30 mL×3), dried over Na₂SO₄, filtered and concentrated in vacuum. The residue was purified by flash silica gel chromatography (20 g SepaFlash® Silica Flash Column, Eluent of 0~50% EtOAc/Petroleum ether gradient @ 100 mL/min) to afford tert-butyl (3-((4-methoxybenzyl)(methyl)amino)cyclobutyl)carbamate (3 g, 9.39 mmol, 86% yield) as a yellow oil. MS(M+H)⁺=321.4.

### Step 2. Synthesis of tert-butyl (3-(methylamino)cyclobutyl)carbamate (3)

To a solution of tert-butyl (3-((4-methoxybenzyl)(methyl)amino)cyclobutyl)carbamate (3 g, 9.36 mmol) in EtOH (40 mL) was added Pd/C (1 g, 10% purity) under H₂ atmosphere. The suspension was degassed and purged with H₂ for 3 times and stirred under H₂ (50 Psi) at 25° C. for 16 h. LCMS showed the starting material was consumed completely. The reaction mixture was filtered and washed by EtOH (50 mL). The filtrate was concentrated in vacuum to afford crude product. The crude product was diluted with H₂O (50 mL) and adjust to pH=3 with HCl (1 M) and extracted with EtOAc (30 mL×3), The aqueous phase was lyophilized to afford tert-butyl (3-(methylamino) cyclobutyl)carbamate (1.58 g, 7.89 mmol, 98.75% yield) as light yellow solid. MS(M+H)⁺=201.1.

### Step 3. Synthesis of tert-butyl (3-(((1-(4-(2,6-dioxopiperidin-3-yl)phenyl)piperidin-4-yl)methyl)(methyl)amino)cyclobutyl)carbamate (5)

To a solution of tert-butyl (3-(methylamino)cyclobutyl)carbamate (190 mg, 948.69 mol) and 1-(4-(2,6-dioxopiperidin-3-yl)phenyl)piperidine-4-carbaldehyde (237.45 mg, 790.57 μmol) in DCM (4 mL) was added AcOH (47.48 mg, 790.57 μmol, 45.26 μL) at 20° C. The mixture was stirred at 20° C. for 30 mins, then NaBH(OAc)₃ (502.66 mg, 2.37 mmol) was slowly added at 20° C. and the resulting mixture was stirred at 20° C. for 16 h. LCMS showed the starting material was consumed completely and 42% peak with desired mass. The reaction mixture was diluted with H₂O (40 mL) and adjusted to pH=10 with Na₂CO₃, followed by the extraction with EtOAc (20 mL×3). The combined organic layer was dried over Na₂SO₄, filtered and concentrated in vacuum. The crude product was purified by prep-TLC (SiO₂, DCM:MeOH=10:1) to afford tert-butyl (3-(((1-(4-(2,6-dioxopiperidin-3-yl)phenyl)piperidin-4-yl)methyl)(methyl)amino)cyclobutyl)carbamate (40 mg, 74.28 μmol, 9.40% yield, 90% purity) as an off-white solid. MS(M+H)⁺=485.4.

### Step 4. Synthesis of 3-(4-(4-(((3-aminocyclobutyl)(methyl)amino)methyl)piperidin-1-yl)phenyl)piperidine-2,6-dione (6)

To a solution of tert-butyl (3-(((1-(4-(2,6-dioxopiperidin-3-yl)phenyl)piperidin-4-yl)methyl)(methyl)amino)cyclo butyl)carbamate (40 mg, 82.54 μmol) in dioxane (1 mL) was added HCl/dioxane (4 M, 20.63 μL) at 20° C. The mixture was stirred at 20° C. for 1 h. LCMS showed starting material was consumed completely and desired mass. The reaction mixture was concentrated in vacuum to afford 3-(4-(4-(((3-aminocyclobutyl)(methyl)amino)methyl)piperidin-1-yl)phenyl)piperidine-2,6-dione (40 mg, crude, HCl) as a white solid. MS(M+H)⁺=385.3.

### Step 5. Synthesis of N-(2-chlorophenyl)-4-((2-((4-((3-(((1-(4-(2,6-dioxopiperidin-3-yl)phenyl)piperidin-4-yl) methyl)(methyl)amino)cyclobutyl)carbamoyl)phenyl)amino)-5-fluoropyrimidin-4-yl)amino)benzamide (Compound 11)

To a solution of 4-((4-((4-((2-chlorophenyl)carbamoyl)phenyl)amino)-5-fluoropyrimidin-2-yl)amino)benzoic acid (15.89 mg, 33.26 μmol) in DMF (0.2 mL) were added HATU (18.06 mg, 47.51 μmol) and DIPEA (18.42 mg, 142.53 μmol, 24.83 μL) at 20° C. The mixture was stirred at 20° C. for 1 h, then a solution of 3-(4-(4-(((3-aminocyclobutyl)(methyl)amino)methyl)piperidin-1-yl)phenyl)piperidine-2,6-dione (20 mg, 47.51 μmol, HCl) in DMF (0.1 mL) was added at 20° C. and the resulting mixture was stirred at 20° C. for 1 h. LCMS showed the starting material was consumed completely and the desired mass. The reaction mixture was diluted with H₂O (4 mL) and extracted with EtOAc (2 mL×3). The organic layer was washed with brine (2 mL×3), dried over Na₂SO₄, filtered and concentrated in vacuum. The residue was purified by prep-TLC (SiO₂, DCM:MeOH=10:1), followed by trituration from a mixture solution (10 mL, ACN:H₂O=1:4) and lyophilized to afford N-(2-chlorophenyl)-4-((2-((4-((3-(((1-(4-(2,6-dioxopiperidin-3-yl)phenyl)piperidin-4-yl) methyl)(methyl)amino)cyclobutyl)carbamoyl)phenyl)amino)-5-fluoropyrimidin-4-yl)amino)benzamide (5.1 mg, 5.74 μmol, 10.77% yield, 95% purity) as a white solid. MS(M−H)⁺=843.8.

¹H NMR (400 MHz, DMSO-d₆) δ=10.78 (s, 1H), 9.94 (s, 1H), 9.78-9.69 (m, 1H), 9.60 (s, 1H), 8.35 (br d, J=7.5 Hz, 1H), 8.25 (d, J=3.5 Hz, 1H), 8.07-7.97 (m, 4H), 7.82-7.74 (m, 4H), 7.68-7.62 (m, 1H), 7.55 (dd, J=1.2, 8.0 Hz, 1H), 7.38 (t, J=7.2 Hz, 1H), 7.31-7.24 (m, 1H), 7.02 (d, J=8.7 Hz, 2H), 6.87 (d, J=8.6 Hz, 2H), 4.14-4.04 (m, 1H), 3.71 (dd, J=5.2, 10.9 Hz, 1H), 3.64 (br d, J=11.1 Hz, 2H), 2.67-2.58 (m, 3H), 2.45-2.41 (m, 1H), 2.41-2.34 (m, 2H), 2.18-2.08 (m, 1H), 2.04 (s, 3H), 2.01-1.98 (m, 1H), 1.88-1.73 (m, 4H), 1.65-1.51 (m, 1H), 1.32-1.23 (m, 3H), 1.20-1.10 (m, 2H).

### Example 12. Synthesis of N-(2-chlorophenyl)-4-((2-((4-((1-(3-(1-(4-(2,6-dioxopiperidin-3-yl)phenyl)piperidin-4-yl)propanoyl)piperidin-4-yl)carbamoyl)phenyl)amino)-5-fluoropyrimidin-4-yl)amino)benzamide (Compound 12)

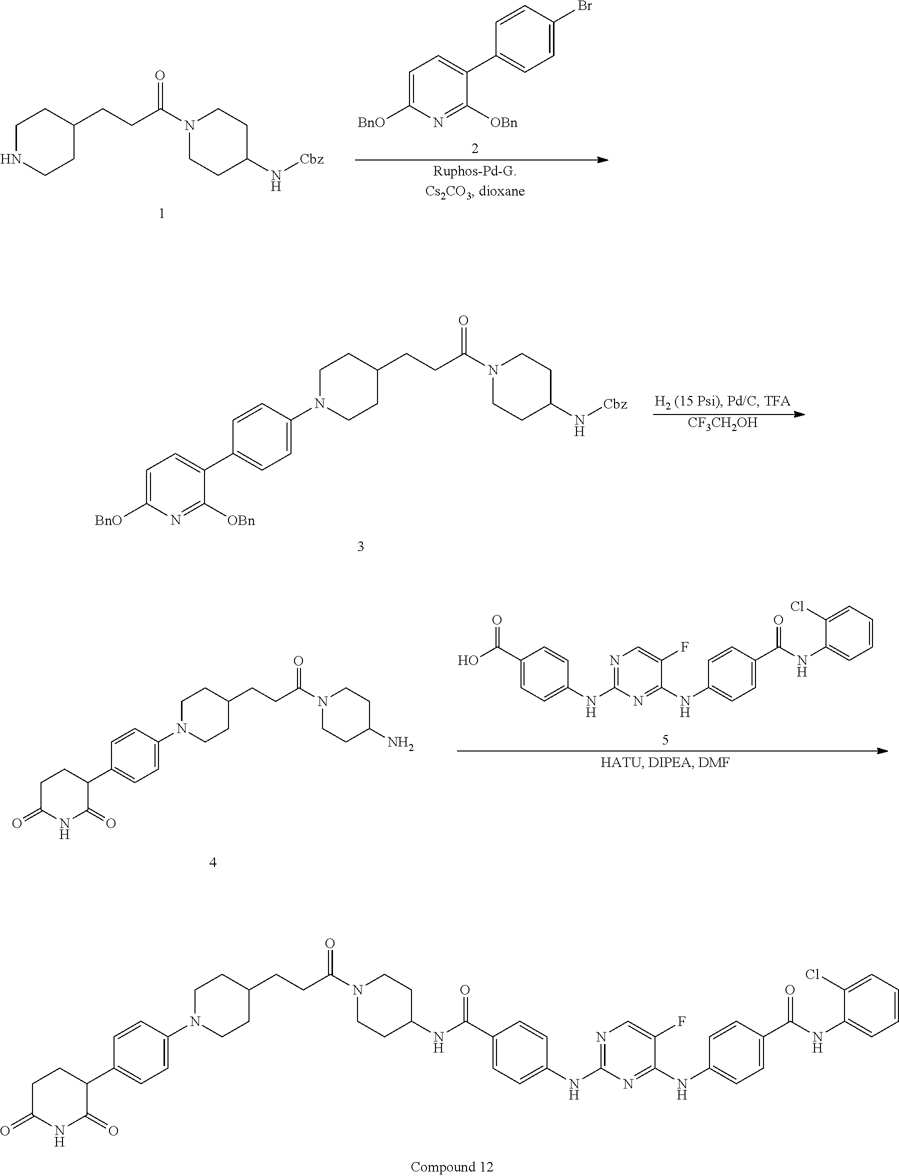

### Step 1. Synthesis of benzyl (1-(3-(1-(4-(2,6-bis(benzyloxy)pyridin-3-yl)phenyl)piperidin-4-yl)propanoyl)piperidin-4-yl)carbamate (3)

To a solution of benzyl (1-(3-(piperidin-4-yl)propanoyl)piperidin-4-yl)carbamate (1.50 g, 3.66 mmol, HCl) and 2,6-bis(benzyloxy)-3-(4-bromophenyl)pyridine (1 g, 2.24 mmol) in dioxane (80 mL) were added Ruphos-Pd-G (190.53 mg, 224.05 μmol) and Cs₂CO₃ (2.19 g, 6.72 mmol). The resulting mixture was stirred at 100° C. for 32 hr under N₂. LCMS showed a peak (17%) with desired mass. The reaction mixture was diluted with water (50 mL) and extracted with EtOAc (30 mL×3). The combined organic layers were washed with saturated brine (100 mL), dried over anhydrous Na₂SO₄, filtered and concentrated under reduced pressure. The residue was purified by flash silica gel chromatography (ISCO®; 40 g SepaFlash® Silica Flash Column, Eluent of 0-100% EtOAc/Petroleum ether gradient @ 80 mL/min) to afford benzyl (1-(3-(1-(4-(2,6-bis(benzyloxy)pyridin-3-yl)phenyl)piperidin-4-yl)propanoyl)piperidin-4-yl)carbamate (0.5 g, 534.57 μmol, 23.86% yield, 79% purity) as a yellow solid. MS(M+H)⁺=739.4.

### Step 2. Synthesis of 3-(4-(4-(3-(4-aminopiperidin-1-yl)-3-oxopropyl)piperidin-1-yl)phenyl)piperidine-2,6-dione (4)

To a solution of benzyl (1-(3-(1-(4-(2,6-bis(benzyloxy)pyridin-3-yl)phenyl)piperidin-4-yl)propanoyl)piperidin-4-yl)carbamate (0.5 g, 676.67 μmol) in CF₃CH₂OH (20 mL) were added TFA (154.31 mg, 1.35 mmol) and Pd/C (100 mg, 93.97 μmol, 10% purity) under N₂ atmosphere, the suspension was degassed and purged with H₂ for 3 times. The mixture was stirred under H₂ (15 Psi) at 25° C. for 16 h. LCMS showed a main peak with desired mass. The mixture was filtered and the filtrate was concentrated to afford 3-(4-(4-(3-(4-aminopiperidin-1-yl)-3-oxopropyl)piperidin-1-yl)phenyl)piperidine-2,6-dione (0.4 g, crude, TFA) as yellow oil. MS(M+H)⁺=427.4.

### Step 3. Synthesis of N-(2-chlorophenyl)-4-((2-((4-((1-(3-(1-(4-(2,6-dioxopiperidin-3-yl)phenyl)piperidin-4-yl) propanoyl)piperidin-4-yl)carbamoyl)phenyl)amino)-5-fluoropyrimidin-4-yl)amino)benzamide (Compound 12)

To a solution of 4-((4-((4-((2-chlorophenyl)carbamoyl)phenyl)amino)-5-fluoropyrimidin-2-yl)amino)benzoic acid (176.80 mg, 369.98 μmol) and HATU (168.81 mg, 443.97 mol) in DMF (5 mL) was added DIPEA (239.08 mg, 1.85 mmol) and the mixture was stirred at 20° C. for 1 h. Then 3-(4-(4-(3-(4-aminopiperidin-1-yl)-3-oxopropyl)piperidin-1-yl)phenyl)piperidine-2,6-dione (200 mg, 369.98 μmol, TFA) was added and the resulting mixture was stirred at 20° C. for 12 h. LCMS showed a peak (50%) with desired mass. The reaction mixture was diluted with water (20 mL) and extracted with EtOAc (10 mL×3). The combined organic layers were washed with saturated brine (10 mL), dried over anhydrous Na₂SO₄, filtered and concentrated under reduced pressure. The residue was purified by prep-TLC (DCM/MeOH=10/1) to afford 80 mg of crude product, which was further purified by prep-HPLC (column: Waters Xbridge 150*25 mm 10 um; mobile phase: [water (NH₄HCO₃)-ACN]; gradient: 35%-65% B over 10 min) and lyophilized to afford N-(2-chlorophenyl)-4-((2-((4-((1-(3-(1-(4-(2,6-dioxopiperidin-3-yl)phenyl)piperidin-4-yl) propanoyl)piperidin-4-yl)carbamoyl)phenyl)amino)-5-fluoropyrimidin-4-yl)amino)benzamide (21.5 mg, 23.50 μmol, 6.35% yield, 96.9% purity) as a white solid. MS(M+H)⁺=886.6.

¹H NMR (400 MHz, DMSO-d₆) δ=10.77 (s, 1H), 9.93 (s, 1H), 9.74 (s, 1H), 9.62 (s, 1H), 8.26 (d, J=3.5 Hz, 1H), 8.08-7.98 (m, 5H), 7.79-7.74 (m, 4H), 7.66 (dd, J=1.5, 7.9 Hz, 1H), 7.57 (dd, J=1.3, 8.1 Hz, 1H), 7.40 (dt, J=1.3, 7.7 Hz, 1H), 7.34-7.25 (m, 1H), 7.03 (d, J=8.6 Hz, 2H), 6.89 (d, J=8.8 Hz, 2H), 4.44-4.30 (m, 1H), 4.09-3.97 (m, 1H), 3.95-3.84 (m, 1H), 3.76-3.71 (m, 1H), 3.70-3.62 (m, 2H), 3.17-3.06 (m, 1H), 2.65-2.57 (m, 5H), 2.42-2.35 (m, 2H), 2.16-2.08 (m, 1H), 2.05-1.98 (m, 1H), 1.89-1.75 (m, 4H), 1.52-1.45 (m, 2H), 1.43-1.34 (m, 2H), 1.32-1.15 (m, 3H).

### Example 13. Synthesis of N-(2-chlorophenyl)-4-((2-((4-((4-((4-(4-(2,6-dioxopiperidin-3-yl)phenyl)piperazin-1-yl)methyl)phenyl)carbamoyl)phenyl)amino)-5-fluoropyrimidin-4-yl)amino)benzamide (Compound 13)

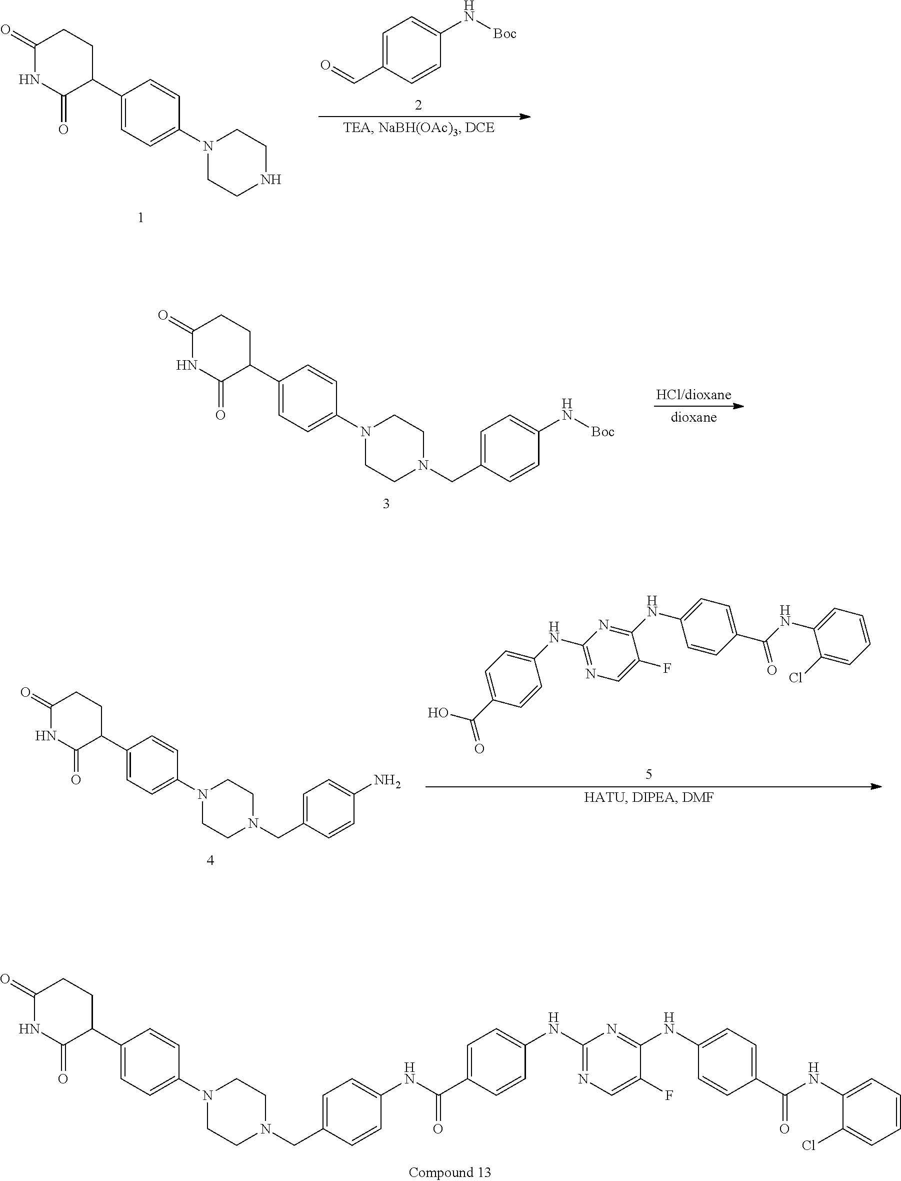 [SMILES: O=C1CCC(c2ccc(N3CCNCC3)cc2)C(=O)N1 ; CC(C)(C)OC(=O)Nc1ccc(C=O)cc1 ; CC(C)(C)OC(=O)Nc1ccc(CN2CCN(c3ccc(C4CCC(=O)NC4=O)cc3)CC2)cc1 ; Nc1ccc(CN2CCN(c3ccc(C4CCC(=O)NC4=O)cc3)CC2)cc1 ; O=C(O)c1ccc(Nc2ncc(F)c(Nc3ccc(C(=O)Nc4ccccc4Cl)cc3)n2)cc1 ; O=C1CCC(c2ccc(N3CCN(Cc4ccc(NC(=O)c5ccc(Nc6ncc(F)c(Nc7ccc(C(=O)Nc8ccccc8Cl)cc7)n6)cc5)cc4)CC3)cc2)C(=O)N1]

### Step 1. Synthesis of tert-butyl (4-((4-(4-(2,6-dioxopiperidin-3-yl)phenyl)piperazin-1-yl)methyl)phenyl)carbamate (3)

To a solution of 3-(4-(piperazin-1-yl)phenyl)piperidine-2,6-dione (300 mg, 968.40 mol HCl) and tert-butyl (4-formylphenyl)carbamate (214.26 mg, 968.40 μmol) in DCE (7 mL) were added TEA (293.97 mg, 2.91 mmol, 404.36 μL) and 4 Å MS (200 mg) at 20′° C. The mixture was stirred at 20° C. for 30 mins, then NaBH(OAc)₃ (615.73 mg, 2.91 mmol) was slowly added and the resulting mixture was stirred at 20° C. for 12 h. LCMS showed starting material was consumed completely and 57% peak with desired mass. The reaction mixture was filtered and the filtrate was concentrated in vacuum. The residue was purified by flash silica gel chromatography (12 g SepaFlash® Silica Flash Column, Eluent of 0~100% EtOAc/Petroleum ether gradient @ 200 mL/min) to afford tert-butyl (4-((4-(4-(2,6-dioxopiperidin-3-yl)phenyl)piperazin-1-yl)methyl)phenyl)carbamate (225 mg, 446.63 μmol, 46.12% yield, 95% purity) as a white solid. MS(M+H)⁺=479.3.

### Step 2. Synthesis of 3-(4-(4-(4-aminobenzyl)piperazin-1-yl)phenyl)piperidine-2,6-dione (4)

To a solution of tert-butyl (4-((4-(4-(2,6-dioxopiperidin-3-yl)phenyl)piperazin-1-yl)methyl)phenyl)carbamate (225 mg, 470.14 μmol) in dioxane (3 mL) was added HCl/dioxane (4 M, 9.20 mL) at 20° C. The mixture was stirred at 20° C. for 4 hrs. LCMS showed 8% of tert-butyl (4-((4-(4-(2,6-dioxopiperidin-3-yl)phenyl)piperazin-1-yl)methyl)phenyl)carbamate remained and 83% peak with desired mass. The reaction mixture was concentrated in vacuum to afford 3-(4-(4-(4-aminobenzyl)piperazin-1-yl)phenyl)piperidine-2,6-dione (130 mg, crude, HCl) as a white solid. MS(M+H)⁺=379.3.

### Step 3. Synthesis of N-(2-chlorophenyl)-4-((2-((4-((4-((4-(4-(2,6-dioxopiperidin-3-yl)phenyl)piperazin-1-yl methyl)phenyl)carbamoyl)phenyl)amino)-5-fluoropyrimidin-4-yl)amino)benzamide (Compound 13)

To a solution of 4-((4-((4-((2-chlorophenyl)carbamoyl)phenyl)amino)-5-fluoropyrimidin-2-yl)amino)benzoic acid (92.14 mg, 192.80 μmol) in DMF (2 mL) was added HATU (80.64 mg, 212.09 μmol) and DIPEA (74.75 mg, 578.41 μmol, 100.75 μL) at 20° C. The mixture was stirred at 20° C. for 30 mins, then a solution of 3-(4-(4-(4-aminobenzyl)piperazin-1-yl)phenyl)piperidine-2,6-dione (80 mg, 192.80 mol HCl) in DMF (2 mL) was added and the reaction mixture was stirred at 20° C. for 12 h. LCMS showed starting material was consumed completely and 40% peak with desired mass. The reaction mixture was diluted with H₂O (20 mL) and extracted with EtOAc (10 mL×3). The organic layer was dried over brine (10 mL×3), filtered and concentrated in vacuum. The residue was purified by prep-TLC (SiO₂, DCM:MeOH=10:1), followed by prep-HPLC (column: Phenomenex luna C18 150*25 mm*10 um; mobile phase: [water (TFA)-ACN]; B %: 38%-68%, 10 min) and lyophilized to afford N-(2-chlorophenyl)-4-((2-((4-((4-((4-(4-(2,6-dioxopiperidin-3-yl)phenyl)piperazin-1-yl) methyl)phenyl)carbamoyl)phenyl)amino)-5-fluoropyrimidin-4-yl)amino)benzamide (45.3 mg, 33.14 μmol, 17.19% yield, 94.7% purity, 4TFA) as a white solid. MS(M+H)⁺=838.1.

¹H NMR (400 MHz, MeOD) δ=8.12 (d, J=3.9 Hz, 1H), 8.07-8.00 (m, 2H), 7.99-7.91 (m, 4H), 7.88 (d, J=8.4 Hz, 2H), 7.82-7.76 (m, 3H), 7.55-7.47 (m, 3H), 7.37 (dt, J=1.5, 7.8 Hz, 1H), 7.29-7.22 (m, 1H), 7.18 (d, J=8.7 Hz, 2H), 7.01 (d, J=8.8 Hz, 2H), 4.92-4.87 (m, 4H), 3.93-3.77 (m, 3H), 3.66-3.49 (m, 2H), 3.11-2.96 (m, 2H), 2.75-2.55 (m, 2H), 2.26-2.14 (m, 2H).

### Example 14. Synthesis of N-(2-chlorophenyl)-4-((2-((4-(4-((4-(2,6-dioxopiperidin-3-yl)phenyl)ethynyl)-[1,4′-bi piperidine]-1′-carbonyl)phenyl)amino)-5-fluoropyrimidin-4-yl)amino)benzamide (Compound 14)

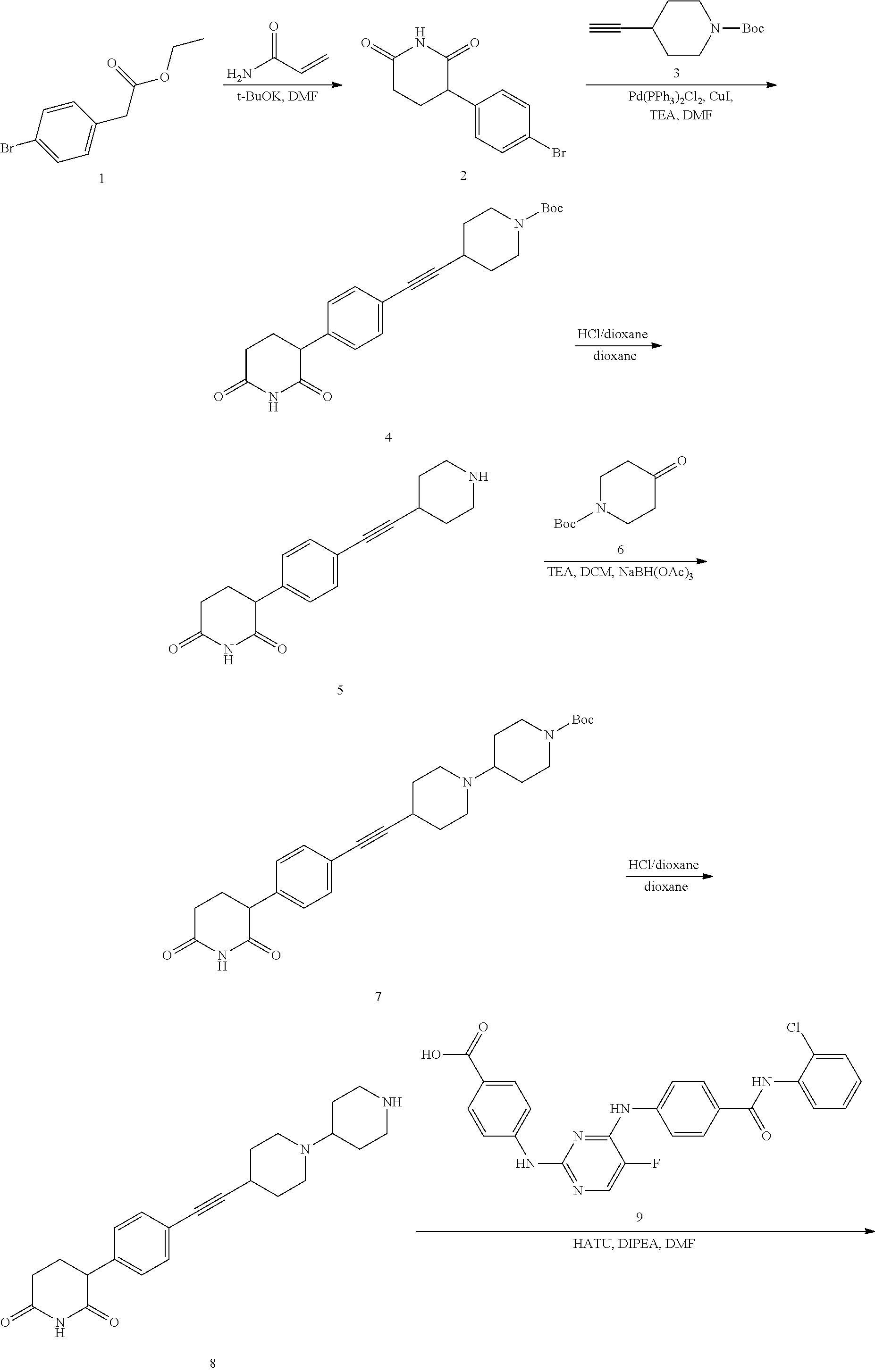

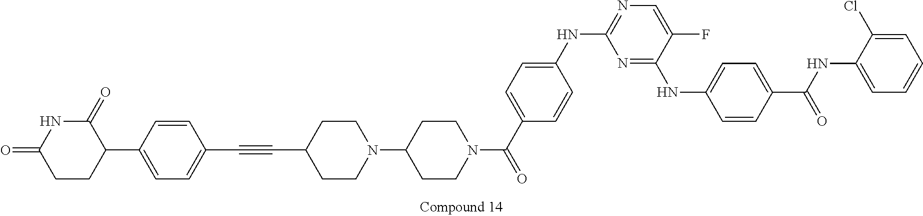 [SMILES: O=C1CCC(c2ccc(C#CC3CCN(C4CCN(C(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of 3-(4-bromophenyl)piperidine-2,6-dione (2)

To a solution of ethyl 2-(4-bromophenyl)acetate (5 g, 21.83 mmol) and acrylamide (1.55 g, 21.83 mmol) in DMF (50 mL) was added t-BuOK (1 M, 21.83 mL) at 0° C., the mixture was stirred at 0° C. for 2 h. TLC (petroleum ether:EtOAc=1:1) showed starting the starting material was consumed completely and a new spot with larger polarity was formed. The reaction mixture was poured into cold water, filtered and washed with H₂O (50 mL). The filter cake was collected and dried to afford 3-(4-bromophenyl)piperidine-2,6-dione (2.5 g, 9.23 mmol, 42.29% yield, 99% purity) as a white solid. MS(M+H)⁺=268.1.

¹H NMR (400 MHz, DMSO-d₆) δ=10.86 (br s, 1H), 7.53 (d, J=8.3 Hz, 2H), 7.20 (d, J=8.4 Hz, 2H), 3.88 (dd, J=4.9, 12.0 Hz, 1H), 2.74-2.62 (m, 1H), 2.56-2.50 (m, 1H), 2.20 (dq, J=4.3, 12.5 Hz, 1H), 2.07-1.96 (m, 1H).

### Step 2. Synthesis of tert-butyl 4-((4-(2,6-dioxopiperidin-3-yl)phenyl)ethynyl)piperidine-1-carboxylate (4)

To a solution of 3-(4-bromophenyl)piperidine-2,6-dione (1.2 g, 4.48 mmol), tert-butyl 4-ethynylpiperidine-1-carboxylate (936.72 mg, 4.48 mmol) and TEA (1.36 g, 13.43 mmol, 1.87 mL) in DMF (10 mL) were added Pd(PPh₃)₂Cl₂ (157.08 mg, 223.79 mol) and CuI (85.24 mg, 447.58 μmol) under N₂. The resulting mixture was stirred at 80° C. for 16 h. LCMS showed 3-(4-bromophenyl)piperidine-2,6-dione was consumed completely and 39% of desired mass. The reaction mixture was diluted with H₂O (50 mL) and extracted with EtOAc (15 mL×3). The combined organic layers were washed with brine (15 mL), dried over Na₂SO₄, filtered and concentrated in vacuum. The residue was purified by flash silica gel chromatography (12 g SepaFlash® Silica Flash Column, Eluent of 0-60% EtOAc/Petroleum ether gradient @ 100 mL/min) to afford tert-butyl 4-((4-(2,6-dioxopiperidin-3-yl)phenyl)ethynyl)piperidine-1-carboxylate (1.8 g) as a yellow solid. MS(M+H)⁺=397.2.

### Step 3. Synthesis of 3-(4-(piperidin-4-ylethynyl)phenyl)piperidine-2,6-dione (5)

To a solution of tert-butyl 4-((4-(2,6-dioxopiperidin-3-yl)phenyl)ethynyl)piperidine-1-carboxylate (1.8 g, 4.54 mmol) in dioxane (10 mL) was added HCl/dioxane (4 M, 20 mL). The resulting solution was stirred at 20° C. for 2 h. LCMS showed the starting material was consumed completely. The reaction mixture was concentrated in vacuum to afford 3-(4-(piperidin-4-ylethynyl)phenyl)piperidine-2,6-dione (1.5 g, HCl salt) as a yellow solid. MS(M+H)⁺=297.2.

### Step 4. Synthesis of tert-butyl 4-((4-(2,6-dioxopiperidin-3-yl)phenyl)ethynyl)-[1,4′-bipiperidine]-1′-carboxylate (7)

A mixture of 3-(4-(piperidin-4-ylethynyl)phenyl)piperidine-2,6-dione (1.4 g, 4.21 mmol, HCl salt), tert-butyl 4-oxopiperidine-1-carboxylate (921.93 mg, 4.63 mmol) and TEA (851.30 mg, 8.41 mmol, 1.17 mL) in DCM (14 mL) was stirred at 20° C. for 0.5 h. Then NaBH(OAc)₃ (2.67 g, 12.62 mmol) was added and the resulting mixture was stirred at 20° C. for 3 h. LCMS showed a 70% peak with desired mass. The reaction mixture was diluted with NaHCO₃ solution (30 mL) and extracted with EtOAc (10 mL×3). The combined organic layers were washed with brine (15 mL), dried over Na₂SO₄, filtered and concentrated in vacuum. The residue was purified by prep-HPLC (column: Phenomenex luna C18 150*40 mm*15 um; mobile phase: [water(TFA)-ACN]; gradient: 20%-50% B over 10 min) and lyophilized to afford tert-butyl 4-((4-(2,6-dioxopiperidin-3-yl)phenyl)ethynyl)-[1,4′-bipiperidine]-1′-carboxylate (1.8 g, 3.45 mmol, 82.08% yield, 92% purity) as a white solid. MS(M+H)⁺=480.3.

### Step 5. Synthesis of 3-(4-([1,4′-bipiperidin]-4-ylethynyl)phenyl)piperidine-2,6-dione (8)

To a solution of tert-butyl 4-((4-(2,6-dioxopiperidin-3-yl)phenyl)ethynyl)-[1,4′-bipiperidine]-1′-carboxylate (0.4 g, 834.01 μmol) in dioxane (4 mL) was added HCl/dioxane (4 M, 4 mL), the mixture was stirred at 20° C. for 2 hr. LCMS showed the starting material was consumed completely. The reaction mixture was concentrated in vacuum to afford 3-(4-([1,4′-bipiperidin]-4-ylethynyl)phenyl)piperidine-2,6-dione (0.36 g, HCl salt) as a yellow solid, which was used directly. MS(M+H)⁺=380.3.

### Step 6. Synthesis of N-(2-chlorophenyl)-4-((2-((4-(4-((4-(2,6-dioxopiperidin-3-yl)phenyl)ethynyl)-[1,4′-bipiperidine]-1′-carbonyl)phenyl)amino)-5-fluoropyrimidin-4-yl)amino)benzamide (Compound 14)

A mixture of 4-((4-((4-((2-chlorophenyl)carbamoyl)phenyl)amino)-5-fluoropyrimidin-2-yl)amino)benzoic acid (93.06 mg, 194.73 μmol), DIPEA (55.93 mg, 432.74 μmol, 75.37 μL) and HATU (90.50 mg, 238.01 μmol) in DMF (1 mL) was stirred at 25° C. for 0.5 h, then a mixture of 3-(4-([1,4′-bipiperidin]-4-ylethynyl)phenyl)piperidine-2,6-dione (90 mg, 216.37 μmol, HCl salt) in DMF (1 mL) was added and the resulting mixture was stirred at 25° C. for 16 h. LCMS showed the starting material was consumed completely and 68% peak with desired mass. The reaction mixture was diluted with H₂O (20 mL) and extracted with THF (10 mL×3). The combined organic layers were washed with brine (10 mL), dried over Na₂SO₄, filtered and concentrated in vacuum. The residue was purified by prep-TLC (SiO₂, DCM:MeOH=7:1) twice to afford N-(2-chlorophenyl)-4-((2-((4-(4-((4-(2,6-dioxopiperidin-3-yl)phenyl)ethynyl)-[1,4′-bipiperidine]-1′-carbonyl)phenyl)amino)-5-fluoropyrimidin-4-yl)amino)benzamide (19.3 mg, 22.07 μmol, 37.06% yield, 96% purity) as a white solid. MS(M+H)⁺=839.2.

¹H NMR (400 MHz, DMSO-d₆) δ=10.84 (s, 1H), 9.91 (s, 1H), 9.72 (s, 1H), 9.55 (s, 1H), 8.23 (d, J=3.5 Hz, 1H), 8.07-7.93 (m, 4H), 7.74 (d, J=8.6 Hz, 2H), 7.66 (dd, J=1.3, 7.9 Hz, 1H), 7.56 (dd, J=1.3, 7.9 Hz, 1H), 7.39 (dt, J=1.3, 7.6 Hz, 1H), 7.37-7.25 (m, 5H), 7.20 (d, J=8.2 Hz, 2H), 4.61-4.09 (m, 1H), 4.09-3.59 (m, 2H), 3.03-2.84 (m, 1H), 2.83-2.71 (m, 2H), 2.70-2.56 (m, 2H), 2.49-2.46 (m, 1H), 2.39-2.29 (m, 2H), 2.28-2.10 (m, 2H), 2.05-1.95 (m, 1H), 1.88-1.68 (m, 4H), 1.63-1.50 (m, 2H), 1.47-1.32 (m, 2H), 1.31-1.20 (m, 1H).

### Example 15. Synthesis of N-(2-chlorophenyl)-4-((2-((4-(2-(4-((4-(2,6-dioxopiperidin-3-yl)phenyl)ethynyl)-[1,4′-bipiperidin]-1′-yl)-2-oxoethyl)phenyl)amino)-5-fluoropyrimidin-4-yl)amino)benzamide (Compound 15)

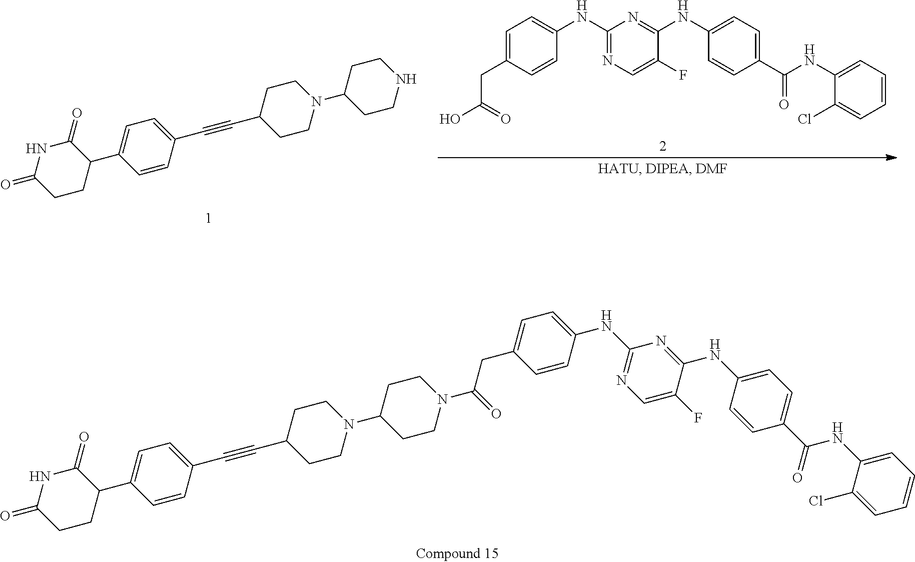 [SMILES: O=C1CCC(c2ccc(C#CC3CCN(C4CCNCC4)CC3)cc2)C(=O)N1 ; O=C(O)Cc1ccc(Nc2ncc(F)c(Nc3ccc(C(=O)Nc4ccccc4Cl)cc3)n2)cc1 ; O=C1CCC(c2ccc(C#CC3CCN(C4CCN(C(=O)Cc5ccc(Nc6ncc(F)c(Nc7ccc(C(=O)Nc8ccccc8Cl)cc7)n6)cc5)CC4)CC3)cc2)C(=O)N1]

### Step 1. Synthesis of N-(2-chlorophenyl)-4-((2-((4-(2-(4-((4-(2,6-dioxopiperidin-3-yl)phenyl)ethynyl)-[1,4′-bipiperidin]-1′-yl)-2-oxoethyl)phenyl)amino)-5-fluoropyrimidin-4-yl)amino)benzamide (Compound 15)

A mixture of 2-(4-((4-((4-((2-chlorophenyl)carbamoyl)phenyl) amino)-5-fluoropyrimidin-2-yl)amino)phenyl)acetic acid (95.79 mg, 194.73 μmol), DIPEA (55.93 mg, 432.74 μmol) and HA TU (90.50 mg, 238.01 μmol) in DMF (1 mL) was stirred at 25° C. for 0.5 h. Then a solution of 3-(4-([1,4′-bipiperidin]-4-ylethynyl)phenyl)piperidine-2,6-dione (90 mg, 216.37 μmol, HCl salt) in DMF (1 mL) was added and the resulting mixture was stirred at 25° C. for 16 h. LCMS showed 3-(4-([1,4′-bipiperidin]-4-ylethynyl)phenyl)piperidine-2,6-dione was consumed completely and 61% peak with desired mass. The reaction mixture was diluted with H₂O (20 mL) and extracted with THF (10 mL×3). The combined organic layers were washed with brine (10 mL), dried over Na₂SO₄, filtered and concentrated under reduced pressure to give a residue. The residue was purified by prep-TLC (SiO₂, DCM:MeOH=10:1) twice to afford N-(2-chlorophenyl)-4-((2-((4-(2-(4-((4-(2,6-dioxopiperidin-3-yl)phenyl)ethynyl)-[1,4′-bipiperidin]-1′-yl)-2-oxoethyl)phenyl)amino)-5-fluoropyrimidin-4-yl)amino)benzamide (17.2 mg, 18.70 μmol, 31.92% yield, 92.8% purity) as a white solid. MS(M+H)⁺=853.2.

¹H NMR (400 MHz, DMSO-d) δ=10.85 (s, 1H), 9.89 (s, 1H), 9.65 (s, 1H), 9.28 (s, 1H), 8.18 (d, J=3.5 Hz, 1H), 8.07-8.00 (m, 2H), 7.99-7.92 (m, 2H), 7.65 (dd, J=1.4, 8.0 Hz, 1H), 7.63-7.58 (m, 2H), 7.56 (dd, J=1.3, 7.9 Hz, 1H), 7.39 (dt, J=1.4, 7.7 Hz, 1H), 7.36-7.26 (m, 3H), 7.19 (d, J=8.2 Hz, 2H), 7.12 (d, J=8.6 Hz, 2H), 4.39 (br dd, J=1.3, 12.0 Hz, 1H), 3.97 (br d, J=11.5 Hz, 1H), 3.87 (dd, J=4.8, 11.6 Hz, 1H), 3.69-3.57 (m, 2H), 2.94 (br t, J=12.3 Hz, 1H), 2.75-2.60 (m, 3H), 2.59-2.54 (m, 1H), 2.47-2.44 (m, 1H), 2.43-2.36 (m, 1H), 2.30-2.11 (m, 3H), 2.06-1.96 (m, 1H), 1.88-1.72 (m, 2H), 1.71-1.59 (m, 2H), 1.59-1.45 (m, 2H), 1.30-1.07 (m, 3H).

### Example 16. Synthesis of N-(2-chlorophenyl)-4-((2-((4-(((1r,4r)-4-((4-(4-(2,6-dioxopiperidin-3-yl)phenyl)piperazin-1-yl)methyl)cyclohexyl)carbamoyl)phenyl)amino)-5-fluoropyrimidin-4-yl)amino)benzamide (Compound 16)

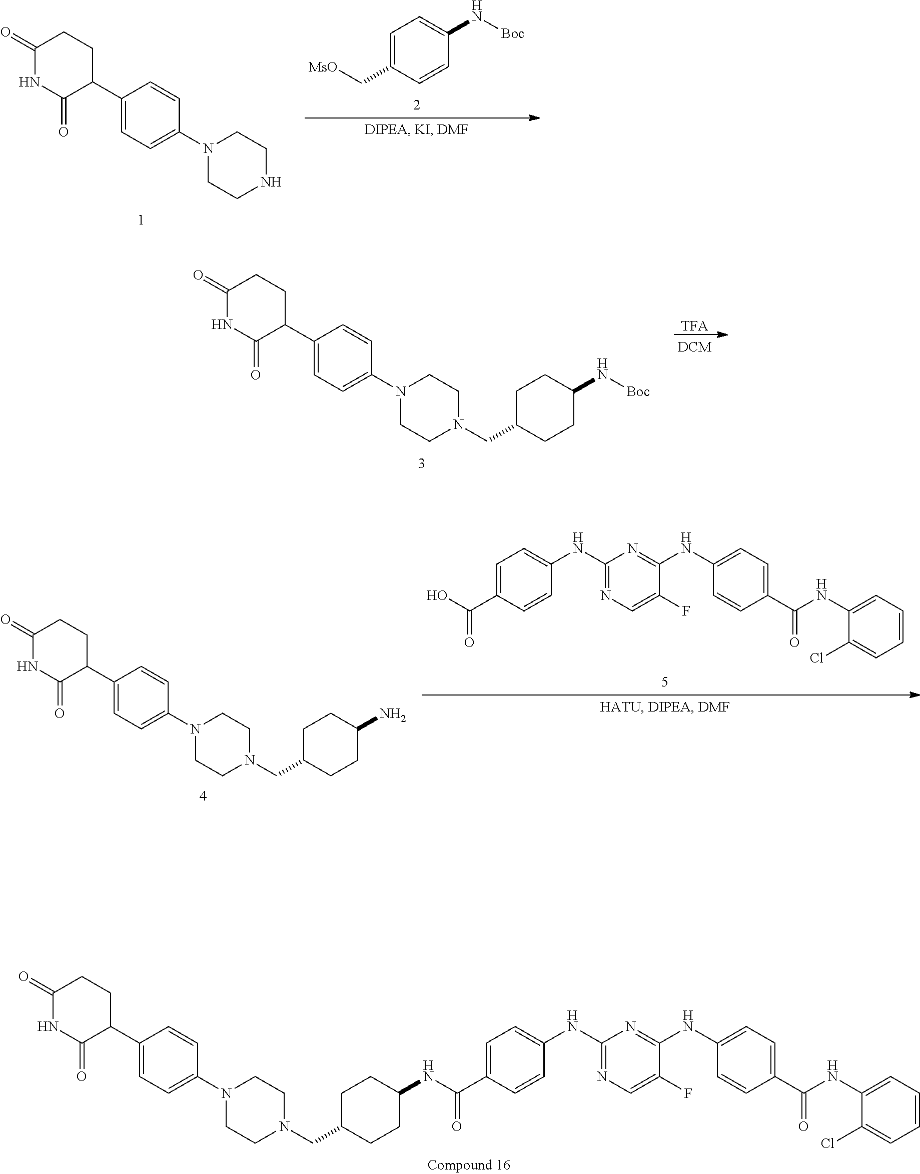

### Step 1. Synthesis of tert-butyl ((1r,4r)-4-((4-(4-(2,6-dioxopiperidin-3-yl)phenyl)piperazin-1-yl)methyl)cyclohexyl)carbamate (3)

A mixture of 3-(4-(piperazin-1-yl)phenyl)piperidine-2,6-dione (300 mg, 866.42 μmol, 2HCl salt), ((1r,4r)-4-((tert-butoxycarbonyl)amino)cyclohexyl)methyl methanesulfonate (266.34 mg. 866.42 μmol). DTPEA (559.90 mg, 4.33 mmol, 754.58 μL) and KI (143.83 mg, 866.42 μmol) in DMF (7 mL) was stirred at 60° C. for 16 hours. LCMS showed 19% of 3-(4-(piperazin-1-yl)phenyl)piperidine-2,6-dione remained and 54% of desired mass was detected. To the mixture was added ((1r,4r)-4-((tert-butoxycarbonyl)amino)cyclohexyl)methyl methanesulfonate (79.90 mg, 259.93 μmol) and then stirred at 60° C. for another 16 hours. LCMS showed 3-(4-(piperazin-1-yl)phenyl)piperidine-2,6-dione was consumed completely and 81% of desired mass was detected. The mixture was treated with CH₃COOH to adjust pH<7. The resulting mixture was purified by prep-HPLC (column: Phenomenex luna C18 150*25 mm*10 um; mobile phase: [water (TFA)-ACN]; gradient: 15%-45% B over 10 min) and lyophilized to afford tert-butyl ((1r,4r)-4-((4-(4-(2,6-dioxopiperidin-3-yl)phenyl)piperazin-1-yl)methyl)cyclohexyl)carbamate (170 mg, 283.97 μmol, 32.78% yield, TFA salt) as a brown solid. MS(M+H)⁺=485.2.

### Step 2. Synthesis of 3-(4-(4-(((1r,4r)-4-aminocyclohexyl)methyl)piperazin-1-yl)phenyl)piperidine-2,6-dione (4)

A mixture of tert-butyl ((1r,4r)-4-((4-(4-(2,6-dioxopiperidin-3-yl)phenyl)piperazin-1-yl)methyl)cyclohexyl)carbamate (125 mg, 208.80 μmol, TFA salt) and TFA (1.54 g, 13.46 mmol, 1 mL) in DCM (4 mL) was stirred at 15° C. for 1 hour. LCMS showed the starting material was consumed completely. The mixture was concentrated in vacuum to afford 3-(4-(4-(((1r,4r)-4-aminocyclohexyl)methyl)piperazin-1-yl)phenyl)piperidine-2,6-dion e (125 mg, 2TFA salt) as a brown gum, which was used directly. MS(M+H)⁺=385.2.

### Step 3. Synthesis of N-(2-chlorophenyl)-4-((2-((4-(((1r,4r)-4-((4-(4-(2,6-dioxopiperidin-3-yl)phenyl)piperazin-1-yl)methyl)cyclohexyl)carbamoyl)phenyl)amino)-5-fluoropyrimidin-4-yl)amino)benzamide (Compound 16)

A mixture of 4-((4-((4-((2-chlorophenyl)carbamoyl)phenyl)amino)-5-fluoropyrimidin-2-yl)amino) benzoic acid (120 mg, 251.11 μmol, HATU (124.12 mg, 326.45 μmol) and DIPEA (324.54 mg, 2.51 mmol) in DMF (5 mL) was stirred at 15° C. for 15 minutes, then 3-(4-(4-(((1r,4r)-4-aminocyclohexyl)methyl)piperazin-1-yl)phenyl)piperidine-2,6-dione (165 mg, 269.36 μmol, 2TFA salt) was added. The resulting mixture was stirred at 15° C. for 1 hour. LCMS showed 3-(4-(4-(((1r,4r)-4-aminocyclohexyl)methyl)piperazin-1-yl)phenyl)piperidine-2,6-dione was consumed completely and 59% of desired mass. The mixture was diluted with H₂O (30 mL) and extracted with a mixture solvents of EtOAc/THF (2/1, 10 mL×2). The combined organic layers were washed with brine (10 mL), dried over Na₂SO₄ and concentrated in vacuum. The residue was purified by prep-HPLC (column: Phenomenex luna C18 150*25 mm*10 um; mobile phase: [water (TFA)-ACN]; gradient: 28%-58% B over 9 min) and lyophilized. The product was diluted with a mixture solvents of EtOAc:THF:ACN (4:2:1, 60 mL) and treated with NaHCO₃ solution (5 mL) to adjust pH>7. The organic phase was dried over Na₂SO₄, filtered and concentrated in vacuum. The residue was purified by prep-TLC (SiO₂, DCM:MeOH=8:1) to afford N-(2-chlorophenyl)-4-((2-((4-(((1r,4r)-4-((4-(4-(2,6-dioxopiperidin-3-yl)phenyl)piperazin-1-yl) methyl)cyclohexyl)carbamoyl)phenyl)amino)-5-fluoropyrimidin-4-yl)amino)benzamide (51.2 mg, 56.94 μmol, 22.67% yield, 93.9% purity) as a light brown solid. MS(M+H)⁺=844.2.

¹H NMR (400 MHz, DMSO-d₆) δ=10.77 (s, 1H), 9.93 (s, 1H), 9.73 (s, 1H), 9.59 (s, 1H), 8.25 (d, J=3.5 Hz, 1H), 8.11-7.94 (m, 5H), 7.80-7.72 (m, 4H), 7.65 (dd, J=1.3, 7.9 Hz, 1H), 7.56 (dd, J=1.3, 7.9 Hz, 1H), 7.40 (dt, J=1.3, 7.7 Hz, 1H), 7.34-7.25 (m, 1H), 7.05 (d, J=8.7 Hz, 2H), 6.89 (d, J=8.7 Hz, 2H), 3.79-3.63 (m, 2H), 3.30 (br s, 1H), 3.15-3.05 (m, 4H), 2.66-2.55 (m, 2H), 2.45-2.40 (m, 2H), 2.21-2.08 (m, 3H), 2.05-1.95 (m, 1H), 1.90-1.75 (m, 4H), 1.55-1.45 (m, 1H), 1.36-1.27 (m, 2H), 1.26-1.19 (m, 1H), 1.02-0.88 (m, 2H).

### Example 17. Synthesis of N-(2-chlorophenyl)-4-((2-((4-(((1r,4r)-4-(2-(4-(4-(2,6-dioxopiperidin-3-yl)phenyl)piperazin-1-yl)ethyl)cyclohexyl)carbamoyl)phenyl)amino)-5-fluoropyrimidin-4-yl)amino)benzamide (Compound 17)

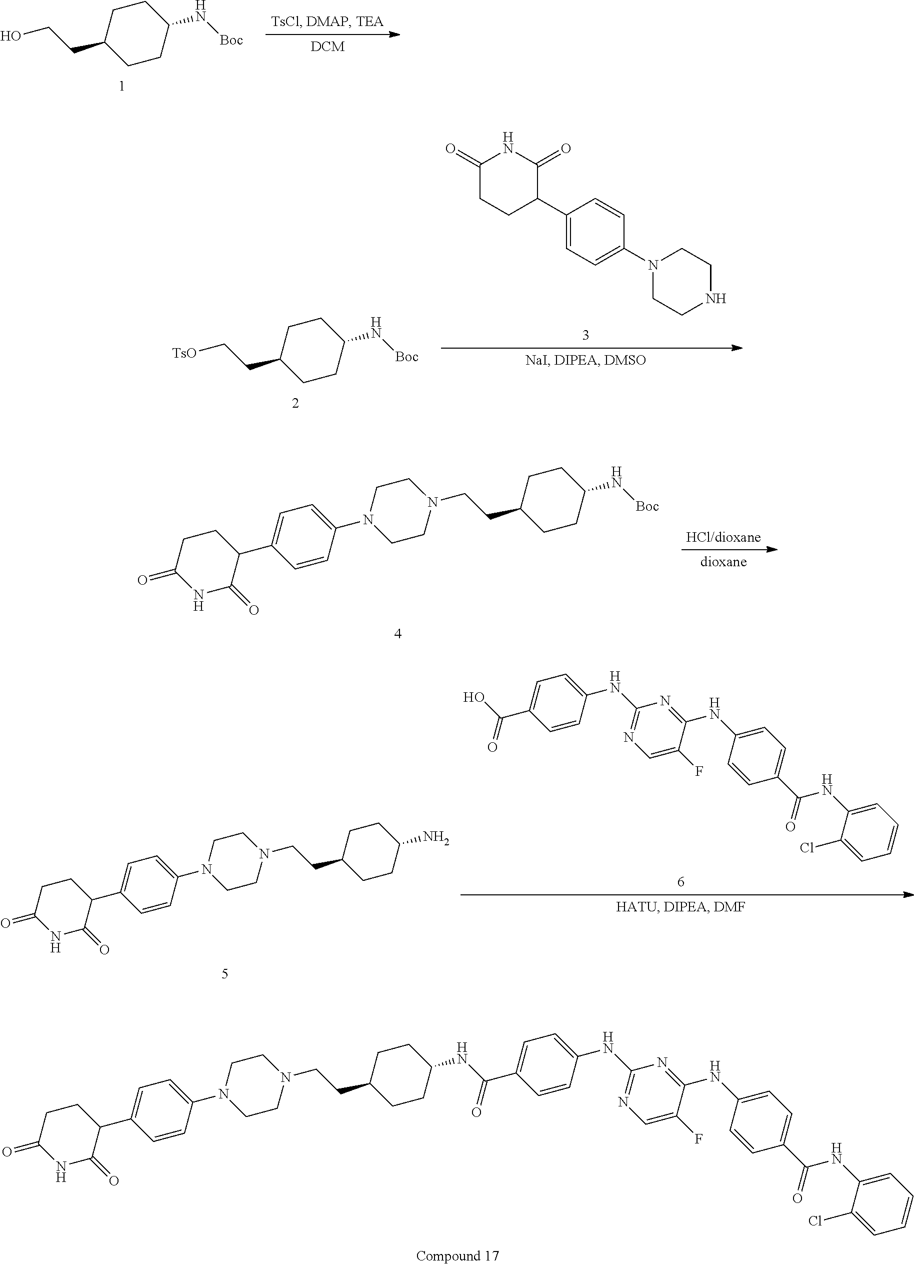

### Step 1. Synthesis of 2-((1r,4r)-4-((tert-butoxycarbonyl)amino)cyclohexyl)ethyl 4-methylbenzenesulfonate (2)

To a solution of tert-butyl ((1r,4r)-4-(2-hydroxyethyl)cyclohexyl)carbamate (2.5 g, 10.27 mmol) in DCM (30 mL) was added TsCl (1.09 g, 15.41 mmol) and TEA (3.12 g, 30.82 mmol, 4.29 mL) and DMAP (125.51 mg, 1.03 mmol). The mixture was stirred at 20° C. for 16 hr. LCMS showed a main peak with desired mass. The reaction mixture was diluted with water (50 mL) and extracted with DCM (30 mL×3). The combined organic layers were washed with water (30 mL), dried over Na₂SO₄, filtered and concentrated under reduced pressure. The residue was purified by flash silica gel chromatography (ISCO®; 20 g SepaFlash® Silica Flash Column, eluent of 20-30% EtOAc/petroleum ether, gradient @ 80 mL/min) to afford 2-((1r,4r)-4-((tert-butoxycarbonyl) amino)cyclohexyl)ethyl 4-methylbenzenesulfonate (1.86 g, 4.68 mmol, 45.54% yield) as a white solid. MS(M+H)⁺=398.1.

### Step 2. Synthesis of tert-butyl ((1r,4r)-4-(2-(4-(4-(2,6-dioxopiperidin-3-yl)phenyl)piperazin-1-yl)ethyl)cyclohexyl)carbamate (4)

To a solution of 3-(4-(piperazin-1-yl)phenyl)piperidine-2,6-dione (2 g, 6.46 mmol, HCl salt) and 2-((1r,4r)-4-((tert-butoxycarbonyl)amino)cyclohexyl)ethyl 4-methylbenzenesulfonate (2.57 g, 6.46 mmol) in DMSO (20 mL) were added NaI (193.54 mg, 1.29 mmol) and DIPEA (4.17 g, 32.28 mmol, 5.62 mL). The resulting mixture was stirred at 80° C. for 16 hr. LCMS showed 42% peak of desired compound was detected. The residue was diluted with water (50 mL) and extracted with EtOAc (30 mL×3). The combined organic layers were washed with brine (20 mL×3), dried over Na₂SO₄, filtered and concentrated under reduced pressure. The crude product was triturated with MTBE at 25° C. for 30 min to afford tert-butyl ((1r,4r)-4-(2-(4-(4-(2,6-dioxopiperidin-3-yl)phenyl)piperazin-1-yl)ethyl)cyclohexyl)carbamate (1.66 g, 3.33 mmol, 51.56% yield) as a yellow solid. MS(M+H)⁺=499.3.

### Step 3. Synthesis of 3-(4-(4-(2-((1r,4r)-4-aminocyclohexyl)ethyl)piperazin-1-yl)phenyl)piperidine-2,6-dione (5)

To a solution of tert-butyl ((1r,4r)-4-(2-(4-(4-(2,6-dioxopiperidin-3-yl)phenyl)piperazin-1-yl)ethyl)cyclohexyl)carbamate (1.77 g, 3.55 mmol) in dioxane (20 mL) was added HCl/dioxane (4 M, 20 mL). The mixture was stirred at 25° C. for 2 hr. LCMS showed one main peak with desired mass. The reaction mixture was concentrated under reduced pressure. The residue was purified by prep-HPLC (column: Phenomenex luna C18 150×40 mm×15 μm; mobile phase: [water(TFA)-ACN]; gradient: 0%-30% B over 10 min) and lyophilized to afford 3-(4-(4-(2-((1r,4r)-4-aminocyclohexyl)ethyl)piperazin-1-yl)phenyl)piperidine-2,6-dion e (1.6 g, 2.98 mmol, 83.93% yield, 81% purity) as a yellow solid. MS(M+H)⁺=399.2.

### Step 4. Synthesis of N-(2-chlorophenyl)-4-((2-((4-(((1r,4r)-4-(2-(4-(4-(2,6-dioxopiperidin-3-yl)phenyl)piperazin-1-yl)ethyl)cyclohexyl)carbamoyl)phenyl)amino)-5-fluoropyrimidin-4-yl)amino)benzamide (Compound 17)

To a solution of 4-((4-((4-((2-chlorophenyl)carbamoyl)phenyl)amino)-5-fluoropyrimidin-2-yl) amino)benzoic acid (800 mg, 1.67 mmol) in DMF (10 mL) were added HATU (954.80 mg, 2.51 mmol), DIPEA (1.08 g, 8.37 mmol, 1.46 mL) and 3-(4-(4-(2-((1r,4r)-4-aminocyclohexyl)ethyl)piperazin-1-yl)phenyl)piperidine-2,6-dione (1.08 g, 2.01 mmol, 81% purity). The resulting mixture was stirred at 25° C. for 2 hr. LCMS showed one main peak with desired mass. The reaction mixture was poured into water (50 mL) and filtered. The filter cake was collected and purified by prep-HPLC (column: Phenomenex luna C18 150×40 mm×15 μm; mobile phase: [water(TFA)-ACN]; gradient: 25%-55% B over 10 min), then lyophilized to afford N-(2-chlorophenyl)-4-((2-((4-(((1r,4r)-4-(2-(4-(4-(2,6-dioxopiperidin-3-yl)phenyl)piperazin-1-yl)ethyl) cyclohexyl)carbamoyl)phenyl)amino)-5-fluoropyrimidin-4-yl)amino)benzamide (849.5 mg, 700.56 μmol, 41.85% yield, 99% purity, 3TFA) as a light yellow solid. MS(M+H)⁺=858.3.

¹H NMR (400 MHz, CD₃OD) δ=8.12 (d, J=4.4 Hz, 1H), 8.02 (br d, J=8.6 Hz, 2H), 7.90 (br d, J=8.6 Hz, 2H), 7.84-7.76 (m, 3H), 7.65 (br d, J=8.5 Hz, 2H), 7.53 (br d, J=8.3 Hz, 1H), 7.39 (br t, J=7.7 Hz, 1H), 7.32-7.25 (m, 1H), 7.19 (br d, J=8.5 Hz, 2H), 7.02 (br d, J=8.4 Hz, 2H), 3.91-3.77 (m, 4H), 3.68 (br d, J=11.4 Hz, 2H), 3.28-3.19 (m, 4H), 3.11-2.97 (m, 2H), 2.76-2.56 (m, 2H), 2.28-2.14 (m, 2H), 2.06-1.96 (m, 2H), 1.91-1.82 (m, 2H), 1.77-1.66 (m, 2H), 1.48-1.33 (m, 3H), 1.27-1.12 (m, 2H).

### Example 18. Synthesis of N-(2-chlorophenyl)-4-((2-((4-((1-(2-(4-(4-(2,6-dioxopiperidin-3-yl)phenyl)piperazin-1-yl)ethyl)piperidin-4-yl)carbamoyl)phenyl)amino)-5-fluoropyrimidin-4-yl)amino) benzamide (Compound 18)

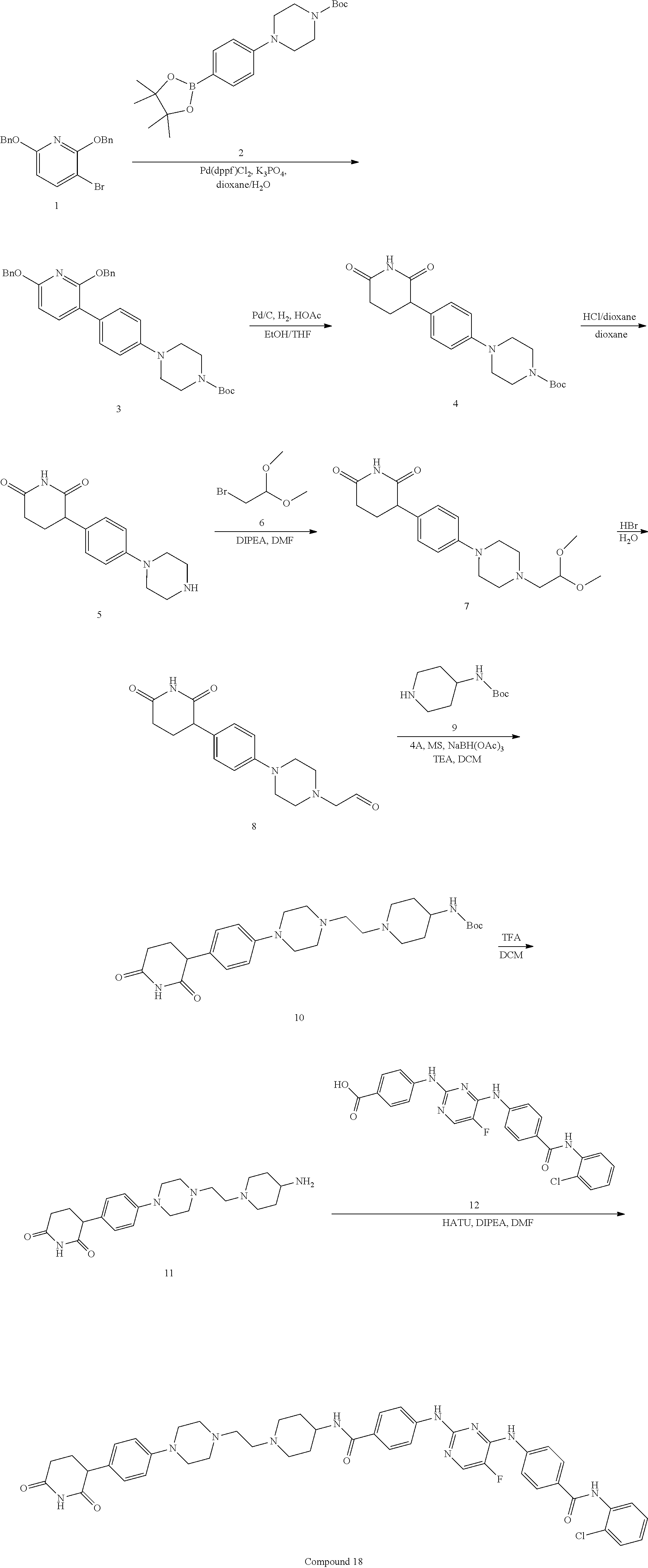

### Step 1. Synthesis of tert-butyl 4-(4-(2,6-bis(benzyloxy)pyridin-3-yl)phenyl)piperazine-1-carboxylate (3)

To a mixture of 2,6-bis(benzyloxy)-3-bromopyridine (5 g, 13.50 mmol), tert-butyl 4-(4-(4,4,5,5-tetramethyl-1,3,2-dioxaborolan-2-yl)phenyl)piperazine-1-carboxylate (5 g, 12.88 mmol) and K₃PO₄ (8.60 g, 40.51 mmol) in dioxane (100 mL) and H₂O (15 mL) was added Pd(dppf)Cl₂ (494.08 mg, 675.24 μmol) at 25° C. The resulting mixture was purged and degassed with N₂ for three times, heated to 100° C. and stirred for 14 h. LCMS showed 2,6-bis(benzyloxy)-3-bromopyridine was consumed completely and one main peak (R_{f}=1.179 min, 82% area) with desired mass was detected. The reaction mixture was diluted with EtOAc (100 mL), dried over Na₂SO₄ and filtered. The filtrate was concentrated in vacuum. The residue was purified by flash silica gel chromatography (40 g silica gel column, EtOAc/petroleum ether=0~6%, 100 mL/min) to afford tert-butyl 4-(4-(2,6-bis(benzyloxy)pyridin-3-yl)phenyl)piperazine-1-carboxylate (7 g, 12.56 mmol, 93.02% yield, 99% purity) as a white solid. MS(M+H)⁺=552.4.

### Step 2. Synthesis of tert-butyl 4-(4-(2,6-dioxopiperidin-3-yl)phenyl)piperazine-1-carboxylate (4)

A mixture of tert-butyl tert-butyl 4-(4-(2,6-bis(benzyloxy)pyridin-3-yl)phenyl)piperazine-1-carboxylate (7 g, 12.69 mmol), HOAc (761.98 mg, 12.69 mmol) and Pd/C (1.35 g, 1.27 mmol, 10% purity) in EtOH (50 mL) and THF (20 mL) was purged and degassed with H₂ for three times and then stirred under H₂ (15 Psi) at 25° C. for 16 h. LCMS showed that the starting material was consumed completely and one main peak (Rf=0.984 min, 96% area) with desired mass was detected. The reaction mixture was filtered, washed with THF (100 mL) and concentrated in vacuum to afford tert-butyl 4-(4-(2,6-dioxopiperidin-3-yl)phenyl)piperazine-1-carboxylate (4.7 g, 11.96 mmol, 94.23% yield, 95% purity) as a gray solid, which was used directly. MS(M+H)⁺=374.2.

### Step 3. Synthesis of 3-(4-(piperazin-1-yl)phenyl)piperidine-2,6-dione (5)

To a solution of tert-butyl 4-(4-(2,6-dioxopiperidin-3-yl)phenyl)piperazine-1-carboxylate (4.7 g, 12.59 mmol) in dioxane (50 mL) was added HCl/dioxane (4 M, 50 mL). The mixture was stirred at 20° C. for 2 h. LCMS showed that the starting material was consumed completely and one main peak (Rf=0.245 min, 95% area) with desired mass was detected. The mixture was concentrated in vacuum to afford 3-(4-(piperazin-1-yl)phenyl)piperidine-2,6-dione (5.8 g, 2HCl salt) as a white solid, which was used directly. MS(M+H)⁺=274.2.

### Step 4. Synthesis of 3-(4-(4-(2,2-dimethoxyethyl)piperazin-1-yl)phenyl)piperidine-2,6-dione (7)

A mixture of 3-(4-(piperazin-1-yl)phenyl)piperidine-2,6-dione (1 g, 2.89 mmol, 2HCl salt), 2-bromo-1,1-dimethoxyethane (634.57 mg, 3.75 mmol, 440.68 μL) and DIPEA (1.87 g, 14.44 mmol, 2.52 mL) in DMF (10 mL) was stirred at 100° C. for 16 hours. LCMS showed 3-(4-(piperazin-1-yl)phenyl)piperidine-2,6-dione was consumed completely and 28% of desired mass was detected. The mixture was diluted with H₂O (30 mL) and treated with saturated Na₂CO₃ solution (30 mL), the resulting mixture was extracted with EtOAc (40 mL×7), the combined organic layers were washed with brine (10 mL×5), dried over Na₂SO₄, filtered and concentrated in vacuum. The residue was purified by flash silica gel chromatography (12 g SepaFlash® Silica Flash Column, eluent of 50-100% EtOAc/petroleum ether to 0~20% MeOH/EtOAc, gradient @ 100 mL/min) to afford 3-(4-(4-(2,2-dimethoxyethyl)piperazin-1-yl)phenyl)piperidine-2,6-dione (450 mg, 1.25 mmol, 22.50% yield) as a brown solid. MS(M+H)⁺=362.2.

### Step 5. Synthesis of 2-(4-(4-(2,6-dioxopiperidin-3-yl)phenyl)piperazin-1-yl)acetaldehyde (8)

A mixture of 3-(4-(4-(2,2-dimethoxyethyl)piperazin-1-yl)phenyl)piperidine-2,6-dione (0.45 g, 1.25 mmol) and HBr (14.90 g, 73.66 mmol, 10 mL, 40% purity) in H₂O (5 mL) was stirred at 40° C. for 16 h. LCMS showed that the starting material was consumed completely and one main peak (Rf=0.421 min, 33% area) with desired mass was detected. The reaction mixture was treated with aqueous Na₂CO₃ solution to adjust pH=8 and extracted with EtOAc (15 mL×5). The combined organic layers were dried over Na₂SO₄, filtered and concentrated in vacuum to afford 2-(4-(4-(2,6-dioxopiperidin-3-yl)phenyl)piperazin-1-yl)acetaldehyde (185 mg, 563.16 mol, 45.23% yield, 96% purity) as a brown solid. MS(M+H)⁺=316.1.

### Step 6. Synthesis of tert-butyl (1-(2-(4-(4-(2,6-dioxopiperidin-3-yl)phenyl)piperazin-1-yl)ethyl)piperidin-4-yl)carbamate (10)

A mixture of 2-(4-(4-(2,6-dioxopiperidin-3-yl)phenyl)piperazin-1-yl)acetaldehyde (160 mg, 507.35 μmol), tert-butyl piperidin-4-ylcarbamate (121.93 mg, 608.82 μmol), 4 Å MS (30 mg) and TEA (51.34 mg, 507.35 μmol, 70.62 μL) in DCM (2 mL) was stirred at 25° C. for 0.5 h. Then NaBH(OAc)₃ (322.58 mg, 1.52 mmol) was added and the resulting mixture was stirred at 25° C. for 2 h. LCMS showed 2-(4-(4-(2,6-dioxopiperidin-3-yl)phenyl)piperazin-1-yl)acetaldehyde was consumed completely and one main peak (R_{f}=0.786 min, 60% area) with desired mass was detected. The reaction mixture was diluted with H₂O (30 mL) and extracted with THF (15 mL×3). The combined organic layers were washed with brine (15 mL), dried over Na₂SO₄, filtered and concentrated in vacuum. The residue was purified by flash silica gel chromatography (4 g SepaFlash® Silica Flash Column, eluent of 50-100% EtOAc/petroleum ether to 0~20% MeOH/EtOAc, gradient @ 100 mL/min) to afford tert-butyl (1-(2-(4-(4-(2,6-dioxopiperidin-3-yl)phenyl)piperazin-1-yl)ethyl)piperidin-4-yl)carbamate (150 mg, 255.18 μmol, 50.30% yield, 85% purity) as a yellow solid. MS(M+H)⁺=500.4.

### Step 7. Synthesis of 3-(4-(4-(2-(4-aminopiperidin-1-yl)ethyl)piperazin-1-yl)phenyl)piperidine-2,6-dione (11)

A mixture of tert-butyl (1-(2-(4-(4-(2,6-dioxopiperidin-3-yl)phenyl)piperazin-1-yl)ethyl)piperidin-4-yl)carbamate (150 mg, 300.21 μmol) and TFA (2.30 g, 20.19 mmol, 1.5 mL) in DCM (5 mL) was stirred at 15° C. for 1 hour. LCMS showed the starting material was consumed completely and desired mass was detected. The mixture was concentrated in vacuum to afford 3-(4-(4-(2-(4-aminopiperidin-1-yl)ethyl)piperazin-1-yl)phenyl)piperidine-2,6-dione (190 mg, 2TFA salt) as a brown gum, which was used directly. MS(M+H)⁺=400.2.

### Step 8. Synthesis of N-(2-chlorophenyl)-4-((2-((4-((1-(2-(4-(4-(2,6-dioxopiperidin-3-yl)phenyl)piperazin-1-yl)ethyl)piperidin-4-yl)carbamoyl)phenyl)amino)-5-fluoropyrimidin-4-yl)amino) benzamide (Compound 18)

A mixture of 4-((4-((4-((2-chlorophenyl)carbamoyl)phenyl)amino)-5-fluoropyrimidin-2-yl)amino) benzoic acid (140 mg, 292.96 μmol), HATU (144.81 mg, 380.85 μmol) and DIPEA (378.64 mg, 2.93 mmol, 510.29 μL) in DMF (2 mL) was stirred at 15° C. for 15 minutes, then 3-(4-(4-(2-(4-aminopiperidin-1-yl)ethyl)piperazin-1-yl)phenyl)piperidine-2,6-dione (190 mg, 302.75 μmol, 2TFA salt) was added and the resulting mixture was stirred at 15° C. for 1 hour. LCMS showed 55% peak of desired mass. The mixture was treated with TFA to adjust pH<7 and filtered. The filtrate was purified by prep-HPLC (column: Phenomenex luna C18 150*40 mm*15 um; mobile phase: [water (TFA)-ACN]; gradient: 10%-40% B over 10 min) and lyophilized. F NMR and H NMR indicated the product was not clean. The impure product was added to a mixture (EtOAc:THF:ACN=4:1:1, 80 mL), the mixture was diluted with H₂O (50 mL) and treated with NaHCO₃ solution (10 mL) to adjust pH>7, the organic phase was separated and concentrated in vacuum. The residue was purified by prep-TLC (SiO₂, DCM:MeOH=8:1) to get the product, which was diluted with a mixture (ACN:H₂O:THF=15:15:4, 34 mL) and freeze-dried to afford N-(2-chlorophenyl)-4-((2-((4-((1-(2-(4-(4-(2,6-dioxopiperidin-3-yl)phenyl)piperazin-1-yl)ethyl)piperidin-4-yl)carbamoyl)phenyl)amino)-5-fluoropyrimidin-4-yl)amino)benzamide (19.9 mg, 22.07 μmol, 7.53% yield, 95.3% purity) as a brown solid. MS(M+H)⁺=859.3.

¹H NMR (400 MHz, DMSO-d₆) δ=10.78 (s, 1H), 9.94 (s, 1H), 9.75 (s, 1H), 9.62 (s, 1H), 8.25 (d, J=3.4 Hz, 1H), 8.12-7.95 (m, 5H), 7.80-7.75 (m, 4H), 7.65 (d, J=7.9 Hz, 1H), 7.56 (d, J=7.9 Hz, 1H), 7.39 (t, J=7.6 Hz, 1H), 7.34-7.25 (m, 1H), 7.05 (br d, J=8.2 Hz, 2H), 6.89 (br d, J=8.6 Hz, 2H), 3.83-3.69 (m, 2H), 3.15-3.07 (m, 4H), 3.03-2.87 (m, 2H), 2.64-2.55 (m, 6H), 2.48-2.38 (m, 4H), 2.20-2.07 (m, 2H), 2.06-1.96 (m, 2H), 1.87-1.74 (m, 2H), 1.69-1.47 (m, 2H).

### Example 19. Synthesis of N-(2-chlorophenyl)-4-((2-((4-((1-(2-(1-(4-(2,6-dioxopiperidin-3-yl)phenyl)piperidin-4-yl)ethyl)piperidin-4-yl)carbamoyl)phenyl)amino)-5-fluoropyrimidin-4-yl)amino) benzamide (Compound 19)

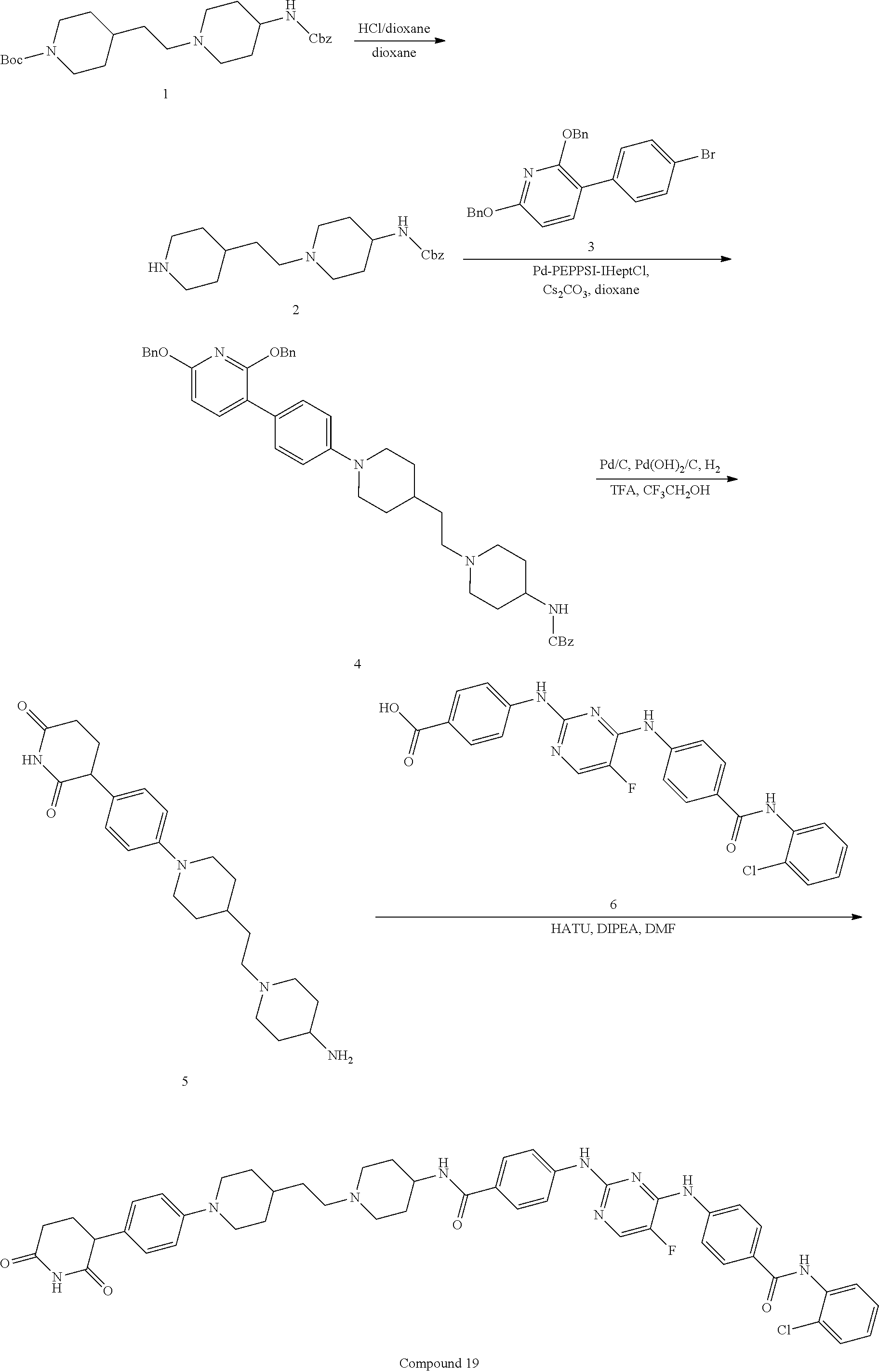

Step 1. Synthesis of benzyl (1-(2-(piperidin-4-yl)ethyl)piperidin-4-yl)carbamate (2)

To a solution of tert-butyl 4-(2-(4-(((benzyloxy)carbonyl)amino)piperidin-1-yl)ethyl)piperidine-1-carboxylate (600 mg, 1.35 mmol) in dioxane (6 mL) was added HCl/dioxane (4 M, 12.00 mL). The mixture was stirred at 25° C. for 0.5 hr. LCMS showed the starting material was consumed completely and desired mass. The reaction mixture was concentrated under reduced pressure to afford benzyl (1-(2-(piperidin-4-yl)ethyl)piperidin-4-yl)carbamate (440 mg, crude, HCl) as a white solid. MS(M+H)⁺=346.2.

### Step 2. Synthesis of benzyl (1-(2-(1-(4-(2,6-bis(benzyloxylpyridin-3-yl)phenyl)piperidin-4-yl)ethyl)piperidin-4-yl) carbamate (4)

A mixture of benzyl (1-(2-(piperidin-4-yl)ethyl)piperidin-4-yl)carbamate (380 mg, 994.92 μmol, HCl), 2,6-bis(benzyloxy)-3-(4-bromophenyl)pyridine (487.99 mg, 994.92 μmol), Cs₂CO₃ (972.50 mg, 2.98 mmol) and Pd-PEPPSI-IHeptCl (48.39 mg, 49.75 μmol) in dioxane (4 mL) was degassed and purged with N₂ for 3 times, then heated to 100° C. and stirred for 16 hrs under N₂ atmosphere. LCMS showed the starting material was consumed completely. The mixture was filtered and concentrated under reduced pressure to give a residue. The residue was purified by flash silica gel chromatography (ISCO®; 20 g SepaFlash® Silica Flash Column, Eluent of 0-10% MeOH/EtOAc @ 80 mL/min) to afford benzyl (1-(2-(1-(4-(2,6-bis(benzyloxy)pyridin-3-yl)phenyl)piperidin-4-yl)ethyl)piperidin-4-yl) carbamate (135 mg, 184.20 μmol, 18.51% yield, 97% purity) as a yellow solid. MS(M+H)⁺=711.5.

### Step 3. Synthesis of 3-(4-(4-(2-(4-aminopiperidin-1-yl)ethyl)piperidin-1-yl)phenyl)piperidine-2,6-dione (5)

To a solution of benzyl (1-(2-(1-(4-(2,6-bis(benzyloxy)pyridin-3-yl)phenyl)piperidin-4-yl)ethyl)piperidin-4-yl) carbamate (135 mg, 189.90 μmol) and TFA (21.65 mg, 189.90 μmol, 14.11 μL) in CF₃CH₂OH (1.3 mL) was added Pd/C (40 mg, 10% purity) and Pd(OH)₂/C (40 mg, 20% purity) under H₂. The mixture was degassed and purged with H₂ several times and stirred under H₂ (15 psi) at 25° C. for 16 hr. LCMS showed the starting material was consumed completely. The mixture was filtered and the filtrate was concentrated under reduced pressure to afford 3-(4-(4-(2-(4-aminopiperidin-1-yl)ethyl)piperidin-1-yl)phenyl)piperidine-2,6-dione (97 mg, 189.24 μmol, 99.66% yield, TFA) as a brown oil. MS(M+H)⁺=399.2.

### Step 4. Synthesis of N-(2-chlorophenyl)-4-((2-((4-((1-(2-(1-(4-(2,6-dioxopiperidin-3-yl)phenyl)piperidin-4-yl) ethyl)piperidin-4-yl)carbamoyl)phenyl)amino)-5-fluoropyrimidin-4-yl)amino) benzamide (Compound 19)

To a solution of 4-((4-((4-((2-chlorophenyl)carbamoyl)phenyl)amino)-5-fluoropyrimidin-2-yl)amino)benzoic acid (60.60 mg, 126.81 μmol) in DMF (0.5 mL) was added HATU (72.33 mg, 190.22 μmol) and DIPEA (81.95 mg, 634.07 μmol, 110.44 μL). The mixture was stirred at 25° C. for 0.5 hr. Then 3-(4-(4-(2-(4-aminopiperidin-1-yl)ethyl)piperidin-1-yl)phenyl)piperidine-2,6-dione (65 mg, 126.81 μmol, TFA) was added and the resulting mixture was stirred at 25° C. for 16 hrs. LCMS showed 3-(4-(4-(2-(4-aminopiperidin-1-yl)ethyl)piperidin-1-yl)phenyl)piperidine-2,6-dione was consumed completely and desired mass. The reaction mixture was poured into H₂O (15 mL) and extracted with EtOAc (10 mL×3). The combined organic layers were washed with brine 10 mL, dried over Na₂SO₄, filtered and concentrated under reduced pressure to give a residue. The residue was purified by flash silica gel chromatography (ISCO®; 20 g SepaFlash® Silica Flash Column, Eluent of 0~20% MeOH/EtOAc @50 mL/min), followed by prep-HPLC (column: Phenomenex luna C18 150*25 mm*10 um; mobile phase: [water (TFA)-ACN]; gradient: 22%-52% B over 9 min) and lyophilized to afford N-(2-chlorophenyl)-4-((2-((4-((1-(2-(1-(4-(2,6-dioxopiperidin-3-yl)phenyl)piperidin-4-yl) ethyl)piperidin-4-yl)carbamoyl)phenyl)amino)-5-fluoropyrimidin-4-yl)amino)benzamide (10 mg, 11.53 μmol, 9.09% yield, 99% purity) as a white solid. MS(M+H)⁺=858.3.

¹H NMR (400 MHz, DMSO-d₆) δ=10.76 (br s, 1H), 9.92 (s, 1H), 9.73 (br d, J=2.0 Hz, 1H), 9.60 (s, 1H), 8.24 (d, J=3.5 Hz, 1H), 8.07-7.95 (m, 5H), 7.77 (s, 4H), 7.68-7.63 (m, 1H), 7.59-7.53 (m, 1H), 7.42-7.37 (m, 1H), 7.32-7.26 (m, 1H), 7.03 (d, J=8.7 Hz, 2H), 6.88 (d, J=8.8 Hz, 2H), 3.79-3.68 (m, 2H), 3.64 (br d, J=11.9 Hz, 2H), 2.90-2.81 (m, 2H), 2.64-2.58 (m, 3H), 2.50-2.45 (m, 1H), 2.31-2.28 (m, 1H), 2.17-2.05 (m, 2H), 2.03-1.97 (m, 1H), 1.92 (br t, J=10.5 Hz, 2H), 1.80-1.70 (m, 4H), 1.60-1.50 (m, 2H), 1.46-1.35 (m, 3H), 1.29-1.20 (m, 2H).

### Example 20. Synthesis of N-(2-chlorophenyl)-4-((2-((4-(4-((6-(2,6-dioxopiperidin-3-yl)pyridin-3-yl)ethynyl)-[1,4′-bipiperidine]-1′-carbonyl)phenyl)amino)-5-fluoropyrimidin-4-yl)amino)benzamide (Compound 20)

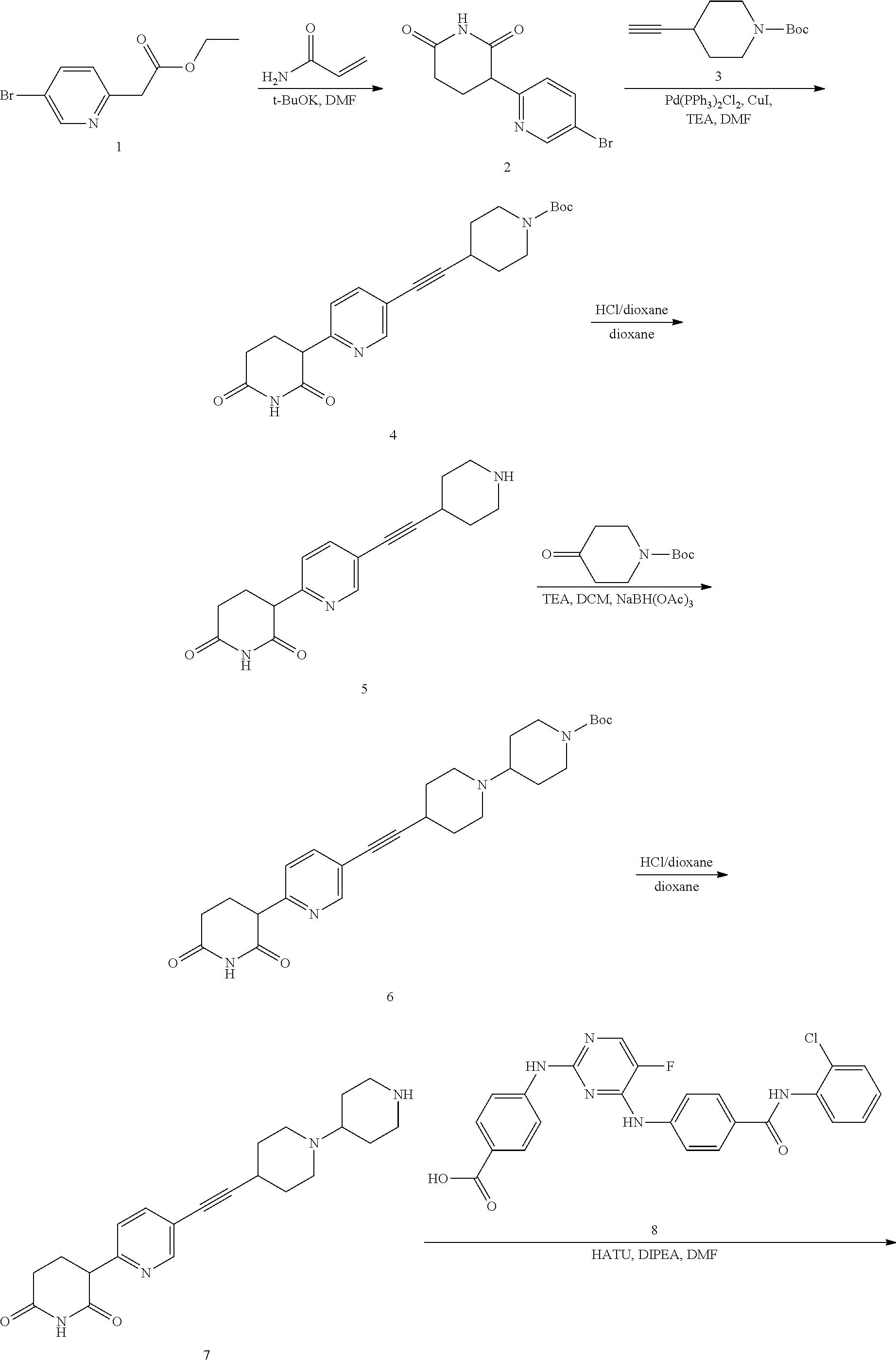

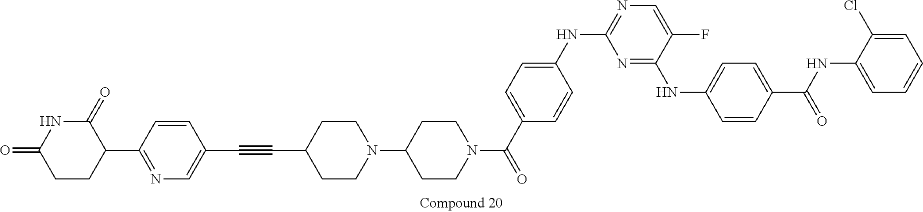 [SMILES: O=C1CCC(c2ccc(C#CC3CCN(C4CCN(C(=O)c5ccc(Nc6ncc(F)c(Nc7ccc(C(=O)Nc8ccccc8Cl)cc7)n6)cc5)CC4)CC3)cn2)C(=O)N1]

### Step 1. Synthesis of 3-(5-bromopyridin-2-yl)piperidine-2,6-dione (2)

To a solution of ethyl 2-(5-bromopyridin-2-yl)acetate (3 g. 13.04 mmol) in DMF (30 mL) were added acrylamide (926.87 mg, 13.04 mmol, 899.87 μL) and t-BuOK (1 M, 13.04 mL) at 0° C. The mixture was stirred at 0° C. for 2 h. TLC (petroleum ether:EtOAc=1:1) showed the start material was consumed and a new spot formed. The mixture was poured into water (50 mL) at 0° C. and extracted with EtOAc (20 mL×3). The combined organic layer was washed with brine (10 mL×3), dried over Na₂SO₄, filtered and concentrated to give a residue, which was triturated by EtOAc/MTBE (20 mL, 1:1) at 20° C. for 1 hr to afford 3-(5-bromopyridin-2-yl)piperidine-2,6-dione (900 mg, 3.34 mmol, 25.65% yield) as a light yellow solid. MS(M+H)⁺=269.0.

### Step 2. Synthesis of tert-butyl 4-((6-(2,6-dioxopiperidin-3-yl)pyridin-3-yl)ethynyl)piperidine-1-carboxylate (4)

To a solution of 3-(5-bromopyridin-2-yl)piperidine-2,6-dione (514.31 mg, 1.91 mmol), tert-butyl 4-ethynylpiperidine-1-carboxylate (0.4 g, 1.91 mmol) and TEA (580.20 mg, 5.73 mmol, 798.08 μL) in DMF (5 mL) was added Pd(PPh₃)₂Cl₂ (67.08 mg, 95.56 μmol) and CuI (36.40 mg, 191.13 μmol) under N₂. The resulting solution was stirred at 80° C. for 16 h. LCMS showed one main peak with desired mass. The reaction mixture was diluted with H₂O (20 mL) and extracted with EtOAc (5 mL×3). The combined organic layers were washed with brine (15 mL), dried over Na₂SO₄, filtered and concentrated under reduced pressure to give a residue. The residue was purified by flash silica gel chromatography (ISCO®; 12 g SepaFlash® Silica Flash Column, Eluent of 0~100% EtOAc/Petroleum ether, gradient @ 100 mL/min) to afford tert-butyl 4-((6-(2,6-dioxopiperidin-3-yl)pyridin-3-yl)ethynyl)piperidine-1-carboxylate (0.78 g, crude) as a yellow solid. MS(M+H)⁺=398.2.

### Step 3. Synthesis of 3-(5-(piperidin-4-ylethynyl)pyridin-2-yl)piperidine-2,6-dione (5)

To a solution of tert-butyl 4-((6-(2,6-dioxopiperidin-3-yl)pyridin-3-yl)ethynyl)piperidine-1-carboxylate (0.78 g, 1.96 mmol) in dioxane (10 mL) was added HCl/dioxane (4 M, 10.00 mL). The resulting mixture was stirred at 20° C. for 2 h. LCMS showed the starting material was consumed completely and one main peak with desired mass. The reaction mixture was concentrated under reduced pressure. The residue was diluted with H₂O (30 mL) and extracted with EtOAc (10 mL×3). The aqueous phase was lyophilized to afford 3-(5-(piperidin-4-ylethynyl)pyridin-2-yl)piperidine-2,6-dione (1 g, crude) as a yellow solid. MS(M+H)⁺=298.2.

### Step 4. Synthesis of tert-butyl 4-((6-(2,6-dioxopiperidin-3-yl)pyridin-3-yl)ethynyl)-[1,4′-bipiperidine]-1′-carboxylate (6)

A mixture of 3-(5-(piperidin-4-ylethynyl)pyridin-2-yl)piperidine-2,6-dione (0.9 g, 2.70 mmol, HCl), tert-butyl 4-oxopiperidine-1-carboxylate (590.91 mg, 2.97 mmol) and TEA (545.64 mg, 5.39 mmol) in DCM (10 mL) was stirred at 20° C. for 0.5 h. Then NaBH(OAc)₃ (1.71 g, 8.09 mmol) was added and the resulting mixture was stirred at 20° C. for 16 h. LCMS showed the starting material was consumed completely. The reaction mixture was diluted with NaHCO₃ solution (30 mL) and extracted with EtOAc (10 mL×3). The combined organic layers were washed with brine (15 mL), dried over Na₂SO₄, filtered and concentrated. The residue was purified by prep-HPLC (column: Phenomenex Luna C18 150*40 mm*15 um; mobile phase: [water (TFA)-ACN]; gradient: 15%-45% B over 10 min) to afford tert-butyl 4-((6-(2,6-dioxopiperidin-3-yl)pyridin-3-yl)ethynyl)-[1,4′-bipiperidine]-1′-carboxylate (180 mg, 269.42 μmol, 9.99% yield, 89% purity, TFA) as a yellow solid. MS(M+H)⁺=481.3.

### Step 5. Synthesis of 3-(5-([1,4′-bipiperidin]-4-ylethynyl)pyridin-2-yl)piperidine-2,6-dione (7)

To a solution of tert-butyl 4-((6-(2,6-dioxopiperidin-3-yl)pyridin-3-yl)ethynyl)-[1,4′-bipiperidine]-1′-carboxylate (180 mg, 374.53 μmol) in dioxane (2 mL) was added HCl/dioxane (4 M, 2 mL). The mixture was stirred at 20° C. for 2 hr. LCMS showed the starting material was consumed completely. The reaction mixture was concentrated under reduced pressure to afford 3-(5-([1,4′-bipiperidin]-4-ylethynyl)pyridin-2-yl)piperidine-2,6-dione (160 mg, crude, HCl) as a yellow solid. MS(M+H)⁺=381.3.

### Step 6. Synthesis of N-(2-chlorophenyl)-4-((2-((4-(4-((6-(2,6-dioxopiperidin-3-yl)pyridin-3-yl)ethynyl)-[1,4′-bipiperidine]-1′-carbonyl)phenyl)amino)-5-fluoropyrimidin-4-yl)amino)benzamide (Compound 20)

A mixture of 4-((4-((4-((2-chlorophenyl)carbamoyl)phenyl)amino)-5-fluoropyrimidin-2-yl) amino)benzoic acid (82.52 mg, 172.69 μmol), DIPEA (49.60 mg, 383.75 μmol, 66.84 μL) and HATU (80.25 mg, 211.06 μmol) in DMF (1 mL) was stirred at 25° C. for 0.5 h. Then a solution of 3-(5-([1,4′-bipiperidin]-4-ylethynyl)pyridin-2-yl)piperidine-2,6-dione (80 mg, 191.87 mol, HCl) in DMF (1 mL) was added and the mixture was stirred at 25° C. for 16 h. LCMS showed one main peak with desired mass. The reaction mixture was diluted with H₂O (20 mL) and extracted with THF (10 mL×3). The combined organic layers were washed with brine (10 mL), dried over Na₂SO₄, filtered and concentrated under reduced pressure to give a residue. The residue was purified by prep-HPLC (column: Phenomenex Luna C18 150*40 mm*15 um; mobile phase: [water (TFA)-ACN]; gradient: 25%-55% B over 10 min), followed by prep-TLC (SiO₂, DCM:MeOH=10:1) to afford N-(2-chlorophenyl)-4-((2-((4-(4-((6-(2,6-dioxopiperidin-3-yl)pyridin-3-yl)ethynyl)-[1,4′-bipiperidine]-1′-carbonyl)phenyl)amino)-5-fluoropyrimidin-4-yl)amino)benzamide (9.3 mg, 10.73 μmol, 5.59% yield, 97% purity) as a white solid. MS(M+H)⁺=840.3.

¹H NMR (400 MHz, DMSO-d₆) δ=10.87 (s, 1H), 9.91 (s, 1H), 9.72 (s, 1H), 9.55 (s, 1H), 8.51 (d, J=1.5 Hz, 1H), 8.22 (d, J=3.7 Hz, 1H), 8.03-7.96 (m, 4H), 7.78 (dd, J=1.9, 8.1 Hz, 1H), 7.73 (br d, J=8.7 Hz, 2H), 7.65 (br d, J=6.8 Hz, 1H), 7.56 (d, J=7.2 Hz, 1H), 7.40-7.27 (m, 5H), 4.05 (dd, J=5.3, 9.7 Hz, 1H), 2.80-2.70 (m, 3H), 2.69-2.65 (m, 1H), 2.37-2.26 (m, 4H), 2.18-2.05 (m, 2H), 2.04-1.94 (m, 1H), 1.89-1.69 (m, 5H), 1.67-1.48 (m, 3H), 1.47-1.32 (m, 3H).

### Example 21. Synthesis of N-(2-chlorophenyl)-4-((2-((4-((7-(1-(4-(2,6-dioxopiperidin-3-yl)-2-fluorophenyl)piperidin-4-yl)-7-azaspiro[3.5]nonan-2-yl)carbamoyl)phenyl)amino)-5-fluoropyrimidin-4-yl)amino)benzamide (Compound 21)

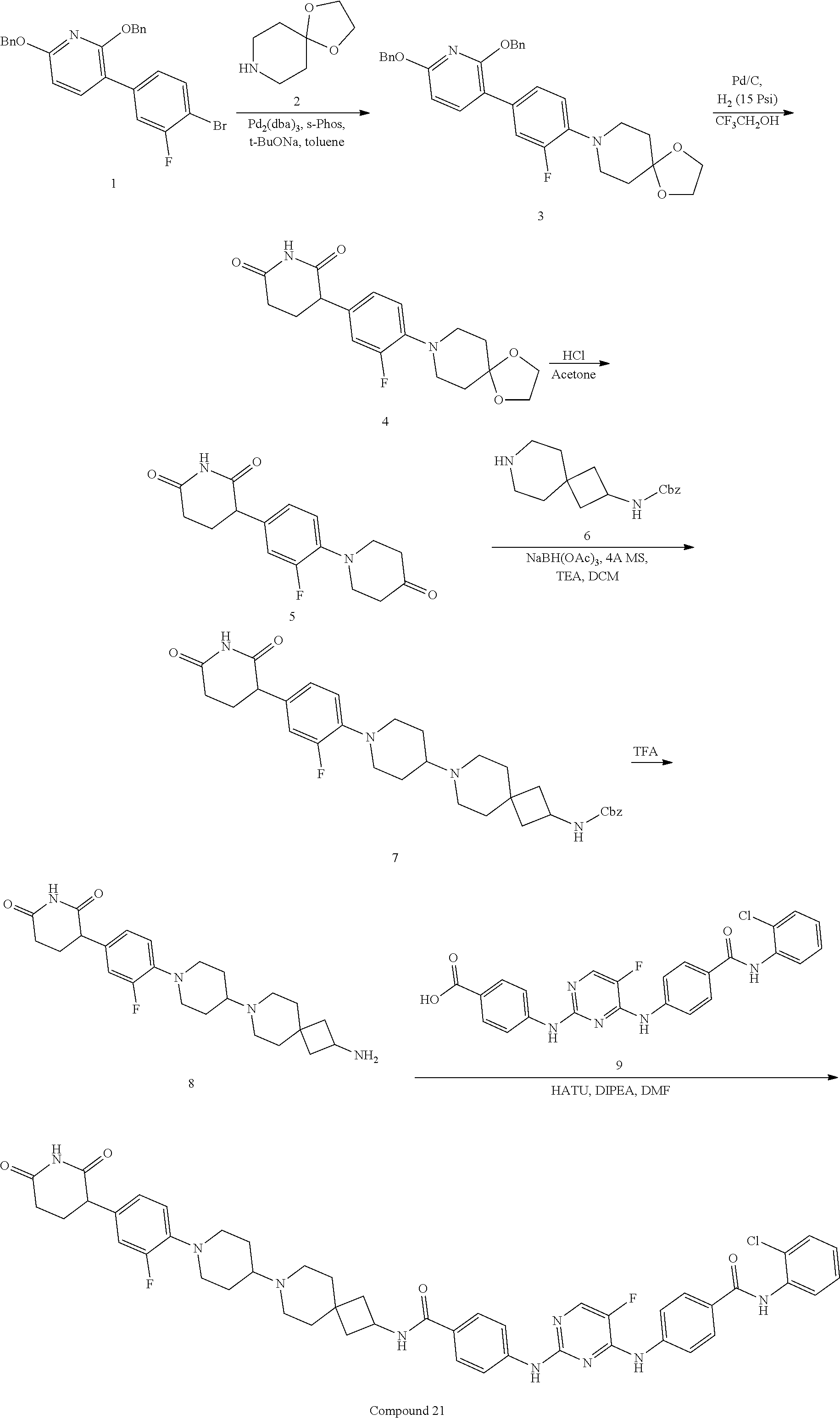

### Step 1. Synthesis of 8-(4-(2,6-bis(benzyloxy)pyridin-3-yl)-2-fluorophenyl)-1,4-dioxa-8-azaspiro[4.5]decane (3)

A mixture of 2,6-bis(benzyloxy)-3-(4-bromo-3-fluorophenyl)pyridine (2 g, 4.31 mmol), 1,4-dioxa-8-azaspiro[4.5]decane (2.00 g, 13.96 mmol, 1.78 mL), Pd₂(dba)₃ (400.00 mg, 436.82 μmol), s-Phos (160.00 mg, 389.74 μmol) and t-BuONa (2 M, 6.01 mL) in toluene (30 mL) was degassed and purged with N₂ for 3 times. The resulting mixture was stirred at 100° C. for 16 hr under N₂ atmosphere. LCMS showed 2,6-bis(benzyloxy)-3-(4-bromo-3-fluorophenyl)pyridine was consumed completely and desired mass. The reaction mixture was quenched by addition H₂O (100 mL) and extracted with EtOAc (30 mL×3). The combined organic layers were dried over Na₂SO₄, filtered and concentrated under reduced pressure to give a residue, which was purified by flash silica gel chromatography (ISCO®; 25 g SepaFlash® Silica Flash Column, Eluent of 0~20% EtOAc/Petroleum ether, gradient @ 50 mL/min) to afford 8-(4-(2,6-bis(benzyloxy)pyridin-3-yl)-2-fluorophenyl)-1,4-dioxa-8-azaspiro[4.5]decane (2.21 g, 3.74 mmol, 86.89% yield, 89.18% purity) as a yellow oil. MS(M+H)⁺=527.3.

### Step 2. Synthesis of 3-(3-fluoro-4-(1,4-dioxa-8-azaspiro[4.5]decan-8-yl)phenyl)piperidine-2,6-dione (4)

To a solution of 8-(4-(2,6-bis(benzyloxy)pyridin-3-yl)-2-fluorophenyl)-1,4-dioxa-8-azaspiro[4.5]decane (2.2 g, 4.18 mmol) in CF₃CH₂OH (30 mL) was added Pd/C (300 mg, 10% purity) under H₂ atmosphere. The suspension was degassed and purged with H₂ for 3 times, and then stirred under H₂ (15 Psi) at 25° C. for 16 hours. LCMS showed 68% peak of 8-(4-(2,6-bis(benzyloxy)pyridin-3-yl)-2-fluorophenyl)-1,4-dioxa-8-azaspiro[4.5]decane remained and 23% of desired compound. The mixture was stirred for another 16 hours. LCMS showed 8-(4-(2,6-bis(benzyloxy)pyridin-3-yl)-2-fluorophenyl)-1,4-dioxa-8-azaspiro[4.5]decane was consumed completely. The reaction mixture was filtered and the filtrate was concentrated to afford 3-(3-fluoro-4-(1,4-dioxa-8-azaspiro[4.5]decan-8-yl)phenyl)piperidine-2,6-dione (1.3 g, 3.73 mmol, 89.32% yield) as a yellow solid. The crude product was used for the next step directly. MS(M+H)⁺=349.2.

### Step 3. Synthesis of 3-(3-fluoro-4-(4-oxopiperidin-1-yl)phenyl)piperidine-2,6-dione (5)

To a solution of 3-(3-fluoro-4-(1,4-dioxa-8-azaspiro[4.5]decan-8-yl)phenyl)piperidine-2,6-dione (0.645 g, 1.85 mmol) in acetone (10 mL) was added HCl (6 M, 1.85 mL). The mixture was stirred at 25° C. for 48 hr. LCMS showed 24% of 3-(3-fluoro-4-(1,4-dioxa-8-azaspiro[4.5]decan-8-yl)phenyl)piperidine-2,6-dione remained and a 63% peak with desired mass. The mixture was diluted with H₂O (20 mL) and neutralized with NaHCO₃ solution, and then extracted with EtOAc (10 mL×3). The combined organic layers were dried over Na₂SO₄, filtered and concentrated under reduced pressure to afford 3-(3-fluoro-4-(4-oxopiperidin-1-yl)phenyl)piperidine-2,6-dione (0.555 g, 1.82 mmol, 98.50% yield) as a blue oil. The crude product was used for the next step directly. MS(M+H)⁺=305.0.

### Step 4. Synthesis of benzyl (7-(1-(4-(2,6-dioxopiperidin-3-yl)-2-fluorophenyl)piperidin-4-yl)-7-azaspiro[3.5]nonan-2-yl)carbamate (7)

To a solution of 3-(3-fluoro-4-(4-oxopiperidin-1-yl)phenyl)piperidine-2,6-dione (0.769 g, 1.26 mmol, 50% purity) in DCM (7 mL) was added benzyl (7-azaspiro[3.5]nonan-2-yl)carbamate (340.99 mg, 1.10 mmol, HCl), 4 Å MS (1.5 g) and TEA (383.56 mg, 3.79 mmol, 527.59 μL). The mixture was stirred at 25° C. for 0.5 hr. And then NaBH(OAc)₃ (803.35 mg, 3.79 mmol) was added and the resulting mixture was stirred at 25° C. for 16 hr. LCMS showed 3-(3-fluoro-4-(4-oxopiperidin-1-yl)phenyl)piperidine-2,6-dione was consumed completely and desired mass. The reaction mixture was poured into water (30 mL) and extracted with DCM (15 mL×3). The combined organic layers were dried over Na₂SO 4, filtered and concentrated under reduced pressure to give a residue, which was purified by flash silica gel chromatography (ISCO®; 20 g SepaFlash® Silica Flash Column, Eluent of 0-30% MeOH/EtOAc @ 50 mL/min) to afford benzyl (7-(1-(4-(2,6-dioxopiperidin-3-yl)-2-fluorophenyl)piperidin-4-yl)-7-azaspiro[3.5]nonan-2-yl)carbamate (0.41 g, 692.23 μmol, 54.79% yield, 95% purity) as a white solid. MS(M+H)⁺=563.3.

### Step 5. Synthesis of 3-(4-(4-(2-amino-7-azaspiro[3.5]nonan-7-yl)piperidin-1-yl)-3-fluorophenyl)piperidine-2,6-dione (8)

A solution of benzyl (7-(1-(4-(2,6-dioxopiperidin-3-yl)-2-fluorophenyl)piperidin-4-yl)-7-azaspiro[3.5]nonan-2-yl)carbamate (56 mg, 99.52 μmol) in TFA (1.72 g, 15.13 mmol, 1.12 mL) was stirred at 40° C. for 16 hr. LC-MS showed benzyl (7-(1-(4-(2,6-dioxopiperidin-3-yl)-2-fluorophenyl)piperidin-4-yl)-7-azaspiro[3.5]nonan-2-yl)carbamate was consumed completely and desired mass. The reaction mixture was concentrated under reduced pressure to afford 3-(4-(4-(2-amino-7-azaspiro[3.5]nonan-7-yl)piperidin-1-yl)-3-fluorophenyl)piperidine-2,6-dione (0.053 g, 97.68 μmol, 98.15% yield, TFA) as a brown oil. The crude product was used for the next step directly. MS(M+H)⁺=429.2.

### Step 6. Synthesis of N-(2-chlorophenyl)-4-((2-((4-((7-(1-(4-(2,6-dioxopiperidin-3-yl)-2-fluorophenyl)piperidin-4-yl)-7-azaspiro[3.5]nonan-2-yl)carbamoyl)phenyl)amino)-5-fluoropyrimidin-4-yl)amino)benzamide (Compound 21)

To a solution of 4-((4-((4-((2-chlorophenyl)carbamoyl)phenyl)amino)-5-fluoropyrimidin-2-yl)amino)benzoic acid (232.94 mg, 365.59 μmol, 75% purity), HATU (208.51 mg, 548.38 μmol) and DIPEA (236.25 mg, 1.83 mmol, 318.40 μL) in DMF (2 mL) was stirred at 25° C. for 0.5 hr. And then 3-(4-(4-(2-amino-7-azaspiro[3.5]nonan-7-yl)piperidin-1-yl)-3-fluorophenyl)piperidine-2,6-dione (0.17 g, 365.59 μmol, HCl) was added and the mixture was stirred at 25° C. for 16 hr. LCMS showed 3-(4-(4-(2-amino-7-azaspiro[3.5]nonan-7-yl)piperidin-1-yl)-3-fluorophenyl)piperidine-2,6-dione was consumed completely and desired mass. The reaction mixture was poured into water (30 mL) and extracted with EtOAc (15 mL×3). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 0-30% MeOH/EtOAc @50 mL/min), followed by prep-HPLC (column: Waters Xbridge 150*25 mm 10 um; mobile phase: [water (NH₄HCO₃)-ACN]; B %: 42%-72%, 10 min) and lyophilized to afford N-(2-chlorophenyl)-4-((2-((4-((7-(1-(4-(2,6-dioxopiperidin-3-yl)-2-fluorophenyl)piperidin-4-yl)-7-azaspiro[3.5]nonan-2-yl)carbamoyl)phenyl)amino)-5-fluoropyrimidin-4-yl)amino)benzamide (9.5 mg, 9.62 μmol, 2.63% yield, 90% purity) as a white solid. MS(M+H)⁺=888.3.

¹H NMR (400 MHz, DMSO-d₆) δ=10.80 (s, 1H), 9.93 (s, 1H), 9.73 (s, 1H), 9.62 (s, 1H), 8.34 (br d, J=7.7 Hz, 1H), 8.25 (d, J=3.4 Hz, 1H), 8.10-7.93 (m, 4H), 7.80-7.76 (m, 4H), 7.69-7.62 (m, 1H), 7.59-7.54 (m, 1H), 7.44-7.36 (m, 1H), 7.33-7.26 (m, 1H), 7.06 (t, J=8.7 Hz, 1H), 6.77-6.65 (m, 2H), 4.43-4.32 (m, 1H), 3.93-3.82 (m, 1H), 3.75-3.68 (m, 2H), 3.32-3.27 (m, 1H), 2.77-2.62 (m, 3H), 2.46-2.41 (m, 2H), 2.40-2.27 (m, 3H), 2.20-2.05 (m, 3H), 2.01-1.89 (m, 1H), 1.80-1.72 (m, 4H), 1.63-1.38 (m, 6H).

### Example 22. Synthesis of N-(2-chlorophenyl)-4-((2-((4-(2-((7-(1-(4-(2,6-dioxopiperidin-3-yl)-2-fluorophenyl)piperidin-4-yl)-7-azaspiro[3.5]nonan-2-yl)amino)-2-oxoethyl)phenyl)amino)-5-fluoropyrimidin-4-yl)amino)benzamide (Compound 22)

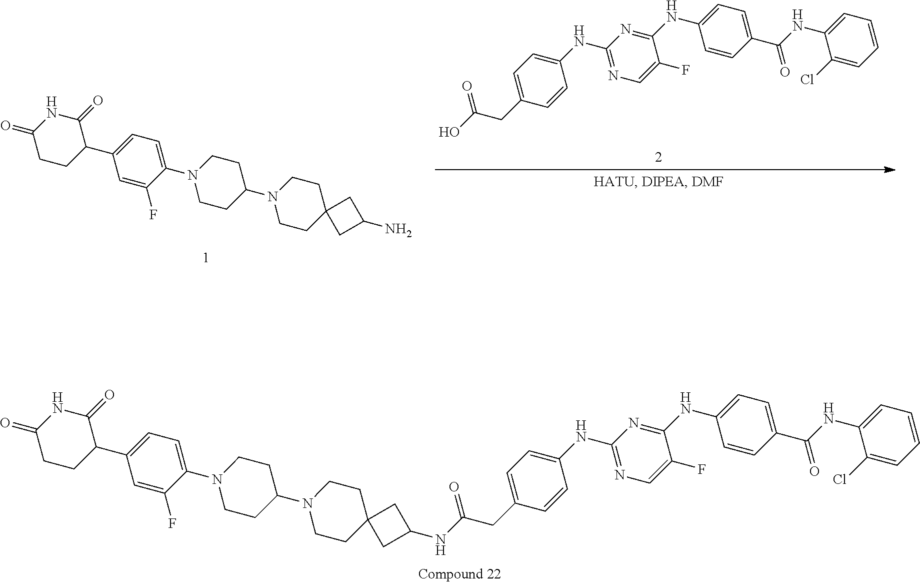 [SMILES: NC1CC2(CCN(C3CCN(c4ccc(C5CCC(=O)NC5=O)cc4F)CC3)CC2)C1 ; O=C(O)Cc1ccc(Nc2ncc(F)c(Nc3ccc(C(=O)Nc4ccccc4Cl)cc3)n2)cc1 ; O=C1CCC(c2ccc(N3CCC(N4CCC5(CC4)CC(NC(=O)Cc4ccc(Nc6ncc(F)c(Nc7ccc(C(=O)Nc8ccccc8Cl)cc7)n6)cc4)C5)CC3)c(F)c2)C(=O)N1]

### Step 1. Synthesis of N-(2-chlorophenyl)-4-((2-((4-(2-((7-(1-(4-(2,6-dioxopiperidin-3-yl)-2-fluorophenyl)piperidin-4-yl)-7-azaspiro[3.5]nonan-2-yl)amino)-2-oxoethyl)phenyl)amino)-5-fluoropyrimidin-4-yl)amino)benzamide (Compound 22)

A mixture of 2-(4-((4-((4-((2-chlorophenyl)carbamoyl)phenyl)amino)-5-fluoropyrimidin-2-yl)amino)phenyl)acetic acid (171.25 mg, 313.33 μmol, 90% purity), HATU (178.70 mg, 469.99 mol) and DIPEA (202.48 mg, 1.57 mmol, 272.88 μL) in DMF (2 mL) was stirred at 25° C. for 0.5 hr. And then 3-(4-(4-(2-amino-7-azaspiro[3.5]nonan-7-yl)piperidin-1-yl)-3-fluorophenyl)piperidine-2,6-dione (0.17 g, 313.33 μmol, TFA) was added and the resulting mixture was stirred at 25° C. for 16 hr. LCMS showed 3-(4-(4-(2-amino-7-azaspiro[3.5]nonan-7-yl)piperidin-1-yl)-3-fluorophenyl)piperidine-2,6-dione was consumed completely and desired mass. The reaction mixture was poured into water (20 mL) and extracted with EtOAc (10 mL×3). The combined organic layers were washed with brine, dried over Na₂SO₄, filtered and concentrated under reduced pressure to give a residue. The residue was purified by flash silica gel chromatography (ISCO®; 20 g SepaFlash® Silica Flash Column, Eluent of 0-30% MeOH/EtOAc @ 80 mL/min), followed by prep-HPLC (column: Waters Xbridge 150*25 mm 10 um; mobile phase: [water (NH₄HCO₃)-ACN]; B %: 42%-72%, 10 min) and lyophilized to afford N-(2-chlorophenyl)-4-((2-((4-(2-((7-(1-(4-(2,6-dioxopiperidin-3-yl)-2-fluorophenyl)piperidin-4-yl)-7-azaspiro[3.5]nonan-2-yl)amino)-2-oxoethyl)phenyl)amino)-5-fluoropyrimidin-4-yl)amino)benzamide (22 mg, 22.18 μmol, 7.08% yield, 91% purity) as a white solid. MS(M+H)⁺=902.3.

¹H NMR (400 MHz, DMSO-d₆) δ=10.80 (s, 1H), 9.91 (s, 1H), 9.65 (s, 1H), 9.25 (s, 1H), 8.31-8.12 (m, 2H), 8.10-7.89 (m, 4H), 7.70-7.50 (m, 4H), 7.45-7.23 (m, 2H), 7.21-6.99 (m, 3H), 6.79-6.61 (m, 2H), 4.12-4.03 (m, 1H), 3.92-3.83 (m, 1H), 3.77-3.65 (m, 2H), 3.43-3.40 (m, 1H), 3.32-3.30 (m, 2H), 2.72-2.60 (m, 3H), 2.47-2.23 (m, 5H), 2.21-2.09 (m, 1H), 2.09-1.86 (m, 3H), 1.81-1.69 (m, 2H), 1.63-1.54 (m, 2H), 1.54-1.38 (m, 6H).

### Example 23. Synthesis of N-(2-chlorophenyl)-4-((2-((4-((7-((1-(4-(2,6-dioxopiperidin-3-yl)-2-fluorophenyl)piperidin-4-yl)methyl)-7-azaspiro[3.5]nonan-2-yl)carbamoyl)phenyl)amino)-5-fluoropyrimidin-4-yl)amino)benzamide (Compound 23)

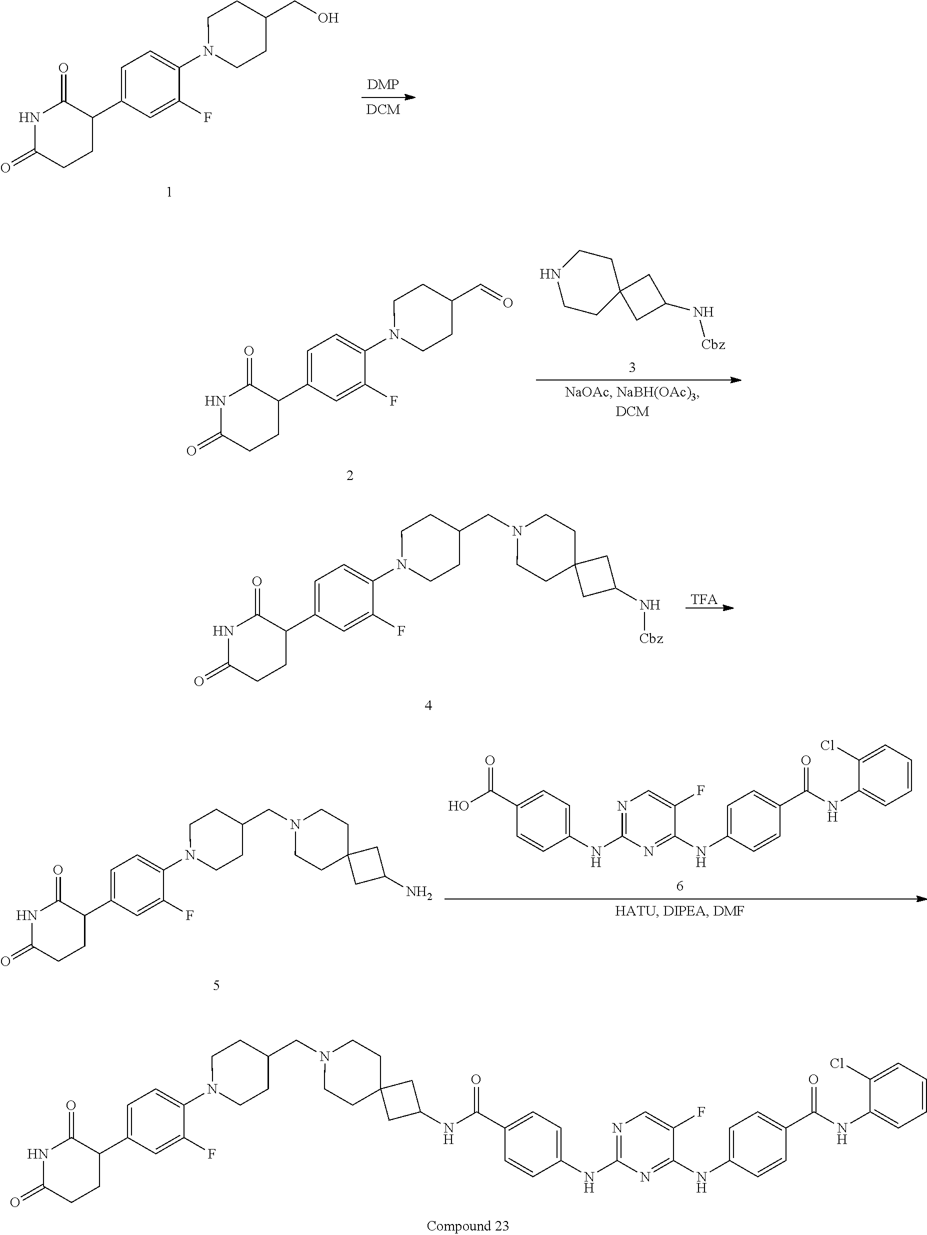

### Step 1. Synthesis of 1-(4-(2,6-dioxopiperidin-3-yl)-2-fluorophenyl)piperidine-4-carbaldehyde (2)

To the solution of 3-(3-fluoro-4-(4-(hydroxymethyl)piperidin-1-yl)phenyl)piperidine-2,6-dione (1.5 g, 4.68 mmol) in DCM (30 mL) was added DMP (2.98 g, 7.02 mmol) and the resulting mixture was stirred at 25° C. for 2 h. TLC (petroleum ether:EtOAc=1:1) showed that 3-(3-fluoro-4-(4-(hydroxymethyl)piperidin-1-yl)phenyl)piperidine-2,6-dione was consumed completely and new spot was formed. The mixture was filtered and the filtrate was concentrated under reduced pressure to afford 1-(4-(2,6-dioxopiperidin-3-yl)-2-fluorophenyl)piperidine-4-carbaldehyde (1.5 g, crude) as yellow oil. MS(M+H)⁺=319.1.

### Step 2. Synthesis of benzyl (7-((1-(4-(2,6-dioxopiperidin-3-yl)-2-fluorophenyl)piperidin-4-yl) methyl)-7-azaspiro[3.5]nonan-2-yl)carbamate (4)

To the solution of 1-(4-(2,6-dioxopiperidin-3-yl)-2-fluorophenyl)piperidine-4-carbaldehyde (1.28 g, 4.02 mmol) and benzyl (7-azaspiro[3.5]nonan-2-yl)carbamate (1.25 g, 4.02 mmol, HCl) in DCM (30 mL) were added NaOAc (494.87 mg, 6.03 mmol) and NaBH(OAc)₃ (1.28 g, 6.03 mmol) and the resulting mixture was stirred at 25° C. for 14 h. LCMS showed a peak (54%) with desired mass. The mixture was poured into water (100 mL) and extracted with DCM (50 mL×3). The combined organic layer was washed by brine (50 mL), dried over Na₂SO₄, filtered and concentrated. The residue was purified by flash silica gel chromatography (ISCO®; 20 g SepaFlash® Silica Flash Column, Eluent of 0~30% MeOH/EtOAc gradient @ 80 mL/min) to afford benzyl (7-((1-(4-(2,6-dioxopiperidin-3-yl)-2-fluorophenyl)piperidin-4-yl)methyl)-7-azaspiro[3.5]nonan-2-yl)carbamate (1.1 g, 1.91 mmol, 47.43% yield) as a yellow solid. MS(M+H)⁺=577.3.

### Step 3. Synthesis of 3-(4-(4-((2-amino-7-azaspiro[3.5]nonan-7-yl)methyl)piperidin-1-yl)-3-fluorophenyl)piperidine-2,6-dione (5)

A mixture of benzyl (7-((1-(4-(2,6-dioxopiperidin-3-yl)-2-fluorophenyl)piperidin-4-yl)methyl)-7-azaspiro[3.5]nonan-2-yl)carbamate (0.5 g, 867.00 μmol) in TFA (7.70 g, 67.53 mmol, 5 mL) was stirred at 40° C. for 17 h. LCMS showed the starting material was consumed completely and 89% desired mass. The mixture was concentrated under reduced pressure to afford 3-(4-(4-((2-amino-7-azaspiro[3.5]nonan-7-yl)methyl)piperidin-1-yl)-3-fluorophenyl)piperidine-2,6-dione (0.4 g, crude) as yellow oil. MS(M+H)⁺=443.3.

### Step 4. Synthesis of N-(2-chlorophenyl)-4-((2-((4-((7-((1-(4-(2,6-dioxopiperidin-3-yl)-2-fluorophenyl)piperidin-4-yl)methyl)-7-azaspiro[3.5]nonan-2-yl)carbamoyl)phenyl)amino)-5-fluoropyrimidin-4-yl)amino)benzamide (Compound 23)

To the solution of 4-((4-((4-((2-chlorophenyl)carbamoyl)phenyl)amino)-5-fluoropyrimidin-2-yl)amino)benzoic acid (287.13 mg, 600.84 μmol) in DMF (5 mL) were added DIPEA (104.49 mg, 808.49 μmol, 140.82 μL) and HATU (122.97 mg, 323.40 μmol), the resulting mixture was stirred at 25° C. for 1 h. Then 3-(4-(4-((2-amino-7-azaspiro[3.5]nonan-7-yl)methyl)piperidin-1-yl)-3-fluorophenyl)piperidine-2,6-dione (150 mg, 269.50 μmol, TFA) was added and the resulting mixture was stirred at 25° C. for 1 h. LCMS showed a main peak with desired mass. The mixture was poured into water (20 mL) and extracted with EtOAc (10 mL×3). The combined organic layer was washed with brine (10 mL), dried over Na₂SO₄, filtered and concentrated. The crude product was purified by prep-HPLC (column: Waters Xbridge C18 150*50 mm*10 μm; mobile phase: [water (NH₄HCO₃)-ACN]; B %: 50%-80%, 10 min), followed by prep-HPLC (column: Phenomenex luna C18 150*25 mm*10 μm; mobile phase: [water (FA)-ACN]; B %: 18%-48%, 10 min) and lyophilized to afford N-(2-chlorophenyl)-4-((2-((4-((7-((1-(4-(2,6-dioxopiperidin-3-yl)-2-fluorophenyl)piperidin-4-yl)methyl)-7-azaspiro[3.5]nonan-2-yl)carbamoyl)phenyl)amino)-5-fluoropyrimidin-4-yl)amino)benzamide (37.4 mg, 39.43 μmol, 14.63% yield, FA) as a white solid. MS(M+H)⁺=902.7.

¹H NMR (400 MHz, DMSO-d₆) δ=10.81 (s, 1H), 9.94 (s, 1H), 9.75 (s, 1H), 9.62 (s, 1H), 8.36 (br d, J=7.1 Hz, 1H), 8.25 (d, J=3.5 Hz, 1H), 8.15 (s, 1H), 8.06-7.98 (m, 4H), 7.79-7.77 (m, 4H), 7.66 (dd, J=1.5, 8.0 Hz, 1H), 7.57 (dd, J=1.4, 8.0 Hz, 1H), 7.40 (dt, J=1.3, 7.7 Hz, 1H), 7.34-7.27 (m, 1H), 7.05-6.92 (m, 3H), 4.44-4.32 (m, 1H), 3.80 (dd, J=4.8, 11.6 Hz, 1H), 3.24-3.18 (m, 2H), 2.66-2.60 (m, 3H), 2.56-2.50 (m, 5H), 2.50-2.54 (m, 2H), 2.21-2.13 (m, 3H), 2.03-1.95 (m, 1H), 1.86-1.76 (m, 4H), 1.71-1.54 (m, 5H), 1.32-1.23 (m, 2H).

### Example 24. Synthesis of N-(2-chlorophenyl)-4-((2-((4-(2-(4-((1-(4-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino)benzamide (Compound 24)

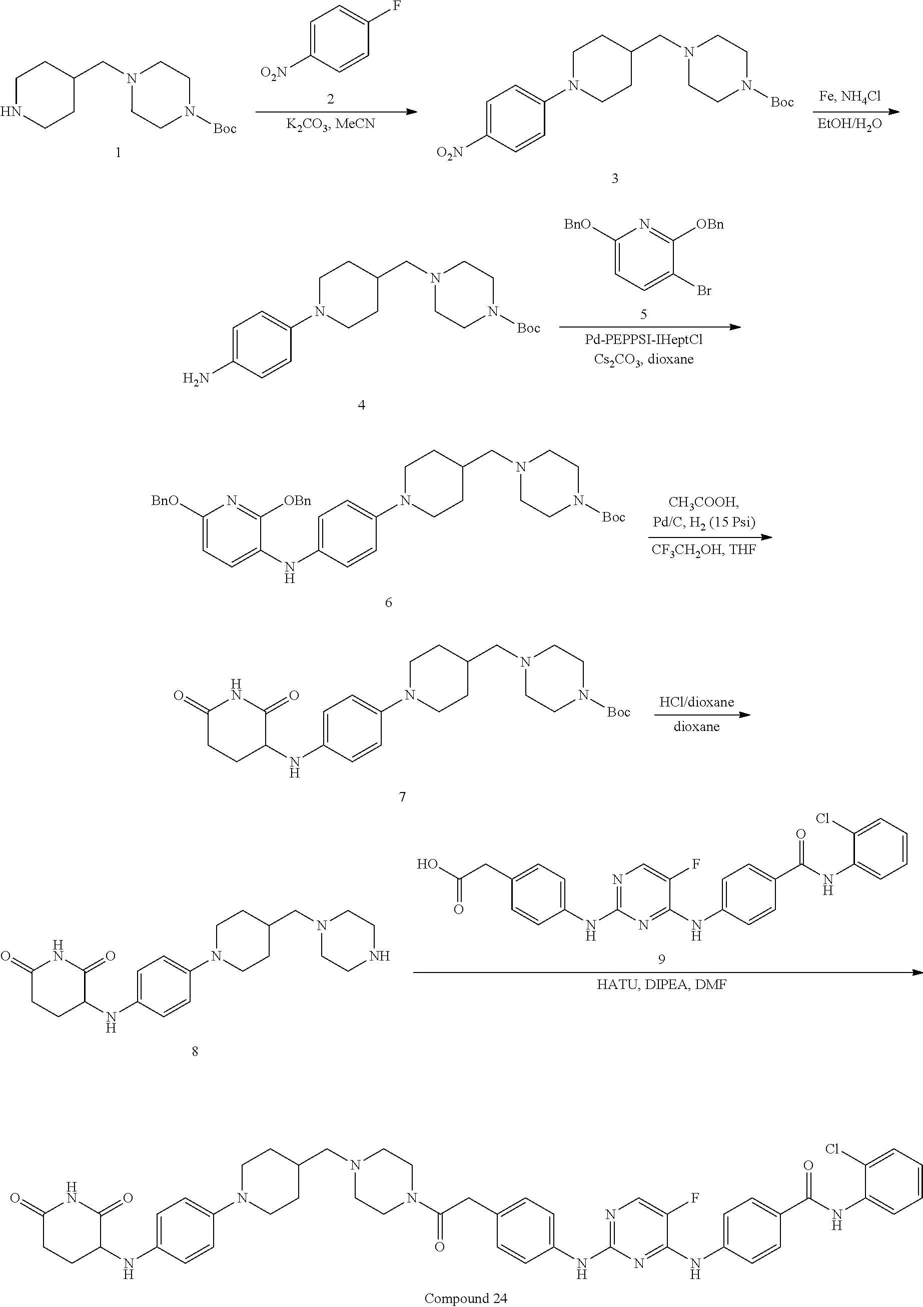

### Step 1. Synthesis of tert-butyl 4-((1-(4-nitrophenyl)piperidin-4-yl)methyl)piperazine-1-carboxylate (3′)

To a solution of 1-fluoro-4-nitrobenzene (1.04 g, 7.37 mmol, 781.48 μL) and tert-butyl 4-(piperidin-4-ylmethyl)piperazine-1-carboxylate (2.3 g, 6.70 mmol, HOAc) in MeCN (30 mL) was added K₂CO₃ (3.70 g, 26.79 mmol). The mixture was stirred at 25° C. for 16 h. LCMS showed a main peak with desired mass. The reaction mixture was filtered and concentrated under reduced pressure to give a residue. The residue was purified by flash silica gel chromatography (ISCO®; 12 g SepaFlash® Silica Flash Column, Eluent of 0~100% EtOAc/Petroleum ether gradient @ 100 mL/min) to afford tert-butyl 4-((1-(4-nitrophenyl)piperidin-4-yl)methyl)piperazine-1-carboxylate (1.9 g, 4.37 mmol, 65.23% yield, 93% purity) as a yellow solid. MS(M+H)⁺=405.2.

### Step 2. Synthesis of tert-butyl 4-((1-(4-aminophenyl)piperidin-4-yl)methyl)piperazine-1-carboxylate (4)

To a mixture of tert-butyl 4-((1-(4-nitrophenyl)piperidin-4-yl)methyl)piperazine-1-carboxylate (1.9 g, 4.70 mmol) and NH₄Cl (1.01 g, 18.79 mmol) in EtOH (10 mL) and H₂O (10 mL) was added Fe (786.93 mg, 14.09 mmol) at 80° C. The resulting mixture was stirred at 80° C. for 1 h. LCMS showed the starting material was consumed completely. The reaction mixture was poured into NaHCO₃ solution (50 mL) and the mixture was extracted with EtOAc (20 mL×3). The combined organic Layers were dried over Na₂SO₄ and concentrated under reduced pressure to give a residue, which was purified by flash silica gel chromatography (ISCO®; 12 g SepaFlash® Silica Flash Column, Eluent of 0-100% EtOAc/Petroleum ether gradient @ 100 mL/min) to afford tert-butyl 4-((1-(4-aminophenyl)piperidin-4-yl)methyl)piperazine-1-carboxylate (1.5 g, 3.80 mmol, 81.00% yield, 95% purity) as a brown solid. MS(M+H)⁺=375.2.

### Step 3. Synthesis of tert-butyl 4-((1-(4-((2,6-bis(benzyloxy)pyridin-3-yl)amino)phenyl)piperidin-4-yl)methyl)piperazine-1-carboxylate (6)

To a mixture of tert-butyl 4-((1-(4-aminophenyl)piperidin-4-yl)methyl)piperazine-1-carboxylate (1.5 g, 4.01 mmol), 2,6-bis(benzyloxy)-3-bromopyridine (1.48 g, 4.01 mmol) and Cs₂CO₃ (3.91 g, 12.02 mmol) in dioxane (20 mL) was added Pd-PEPPSI-IHeptCl (194.80 mg, 200.26 mol). The resulting mixture was stirred at 90° C. under N₂ for 16 h. LCMS showed a main peak with desired mass. The reaction mixture was diluted with H₂O (50 mL) and extracted with EtOAc (10 mL×3). The combined organic layers were washed with brine (10 mL), dried over Na₂SO₄, filtered and concentrated under reduced pressure to give a residue, which was purified by flash silica gel chromatography (ISCO®; 12 g SepaFlash® Silica Flash Column, Eluent of 0-60% EtOAc/Petroleum ether gradient @100 mL/min) to afford tert-butyl 4-((1-(4-((2,6-bis(benzyloxy)pyridin-3-yl)amino)phenyl)piperidin-4-yl)methyl)piperazine-1-carboxylate (2.5 g, 3.62 mmol, 90.27% yield, 96% purity) as a brown oil. MS(M+H)⁺=664.4.

### Step 4. Synthesis of tert-butyl 4-((1-(4-((2,6-dioxopiperidin-3-yl)amino)phenyl)piperidin-4-yl) methyl)piperazine-1-carboxylate (7)

To a solution of tert-butyl 4-((1-(4-((2,6-bis(benzyloxy)pyridin-3-yl)amino)phenyl)piperidin-4-yl)methyl)piperazine-1-carboxylate (1.25 g, 1.88 mmol) in CF₃CH₂OH (40 mL) and THF (8 mL) was added CH₃COOH (113.08 mg, 1.88 mmol) and Pd/C (200.39 mg, 188.30 μmol, 10% purity) under H₂. The resulting mixture was stirred at 25° C. under H₂ (15 psi) for 16 h. LCMS showed the starting material was still remained. Another portion of Pd/C (200.39 mg, 188.30 μmol, 10% purity) was added and the mixture was stirred at 25° C. under H₂ (15 psi) for 6 h. LCMS showed the starting material was consumed completely. The reaction mixture was filtered and concentrated under reduced pressure to afford tert-butyl 4-((1-(4-((2,6-dioxopiperidin-3-yl)amino)phenyl)piperidin-4-yl)methyl)piperazine-1-carboxylate (1.5 g, crude) as a black solid. The crude product was used into the next step without further purification. MS(M+H)⁺=486.3.

### Step 5. Synthesis of 3-((4-(4-(piperazin-1-ylmethyl)piperidin-1-yl)phenyl)amino)piperidine-2,6-dione (8)

To a solution of tert-butyl 4-((1-(4-((2,6-dioxopiperidin-3-yl)amino)phenyl)piperidin-4-yl) methyl)piperazine-1-carboxylate (200 mg, 411.85 μmol) in dioxane (2 mL) was added HCl/dioxane (4 M, 2 mL). The resulting mixture was stirred at 25° C. for 2 h. LCMS showed the starting material was consumed completely. The reaction mixture was concentrated under reduced pressure to afford 3-((4-(4-(piperazin-1-ylmethyl)piperidin-1-yl)phenyl)amino)piperidine-2,6-dione (180 mg, crude, HCl) as a black solid. The crude product was used into the next step without further purification. MS(M+H)⁺=386.4.

### Step 6. Synthesis of N-(2-chlorophenyl)-4-((2-((4-(2-(4-((1-(4-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino)benzamide (Compound 24)

A mixture of 2-(4-((4-((4-((2-chlorophenyl)carbamoyl)phenyl)amino)-5-fluoropyrimidin-2-yl)amino)phenyl)acetic acid (181.51 mg, 368.99 mol), HATU (233.83 mg, 614.98 mol) and DIPEA (158.96 mg, 1.23 mmol, 214.24 μL) in DMF (2 mL) was stirred at 25° C. for 0.5 h. Then a solution of 3-((4-(4-(piperazin-1-ylmethyl)piperidin-1-yl)phenyl)amino)piperidine-2,6-dione (173 mg, 409.99 μmol, HCl) and DIPEA (158.96 mg, 1.23 mmol, 214.24 μL) in DMF (2 mL) was added. The resulting mixture was stirred at 25° C. for 16 h. LCMS showed one main peak with desired mass. The reaction mixture was diluted with H₂O (50 mL) and extracted with THF (10 mL×3). The combined organic layers were washed with brine (15 mL), dried over Na₂SO₄, filtered and concentrated under reduced pressure to give a residue. The residue was purified by flash silica gel chromatography (ISCO®; 12 g SepaFlash® Silica Flash Column, Eluent of 0-100% EtOAc/Petroleum ether gradient to 0~50% MeOH/EtOAc gradient @ 100 mL/min). The residue was further purified by prep-HPLC (column: Phenomenex Luna C18 150*25 mm*10 um; mobile phase: [water (FA)-ACN]; B %: 12%-32%, 10 min), followed by prep-TLC (SiO₂, DCM:MeOH=15:1) to afford N-(2-chlorophenyl)-4-((2-((4-(2-(4-((1-(4-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino)benzamide (7 mg, 7.49 μmol, 1.83% yield, 92% purity) as a yellow solid. MS(M+H)⁺=859.3.

¹H NMR (400 MHz, DMSO-d₆) δ=10.76 (brs, 1H), 9.89 (s, 1H), 9.65 (s, 1H), 9.28 (s, 1H), 8.18 (br s, 1H), 8.06-8.00 (m, 2H), 8.00-7.94 (m, 2H), 7.65 (br d, J=7.9 Hz, 1H), 7.60 (br d, J=7.5 fHz, 2H), 7.56 (br d, J=8.4 Hz, 1H), 7.39 (br t, J=7.7 Hz, 1H), 7.33-7.26 (m, 1H), 7.12 (br d, J=7.8 Hz, 2H), 6.73 (br d, J=7.7 Hz, 2H), 6.58 (br d, J=8.1 Hz, 2H), 5.38-5.32 (m, 1H), 4.24-4.12 (m, 1H), 3.63 (br s, 2H), 3.55-3.45 (m 4H), 2.79-2.69 (m, 1H), 2.60-2.57 (m, 2H), 2.46-2.40 (m, 2H), 2.29-2.16 (m, 4H), 2.15-2.11 (m, 3H), 1.89-1.78 (m, 1H), 1.76-1.67 (m, 2H), 1.54-1.46 (m, 1H), 1.20-1.14 (m, 2H).

### Example 25. Synthesis of N-(2-chlorophenyl)-4-((2-((4-(2-(4-((1-(3-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino)benzamide (Compound 25)

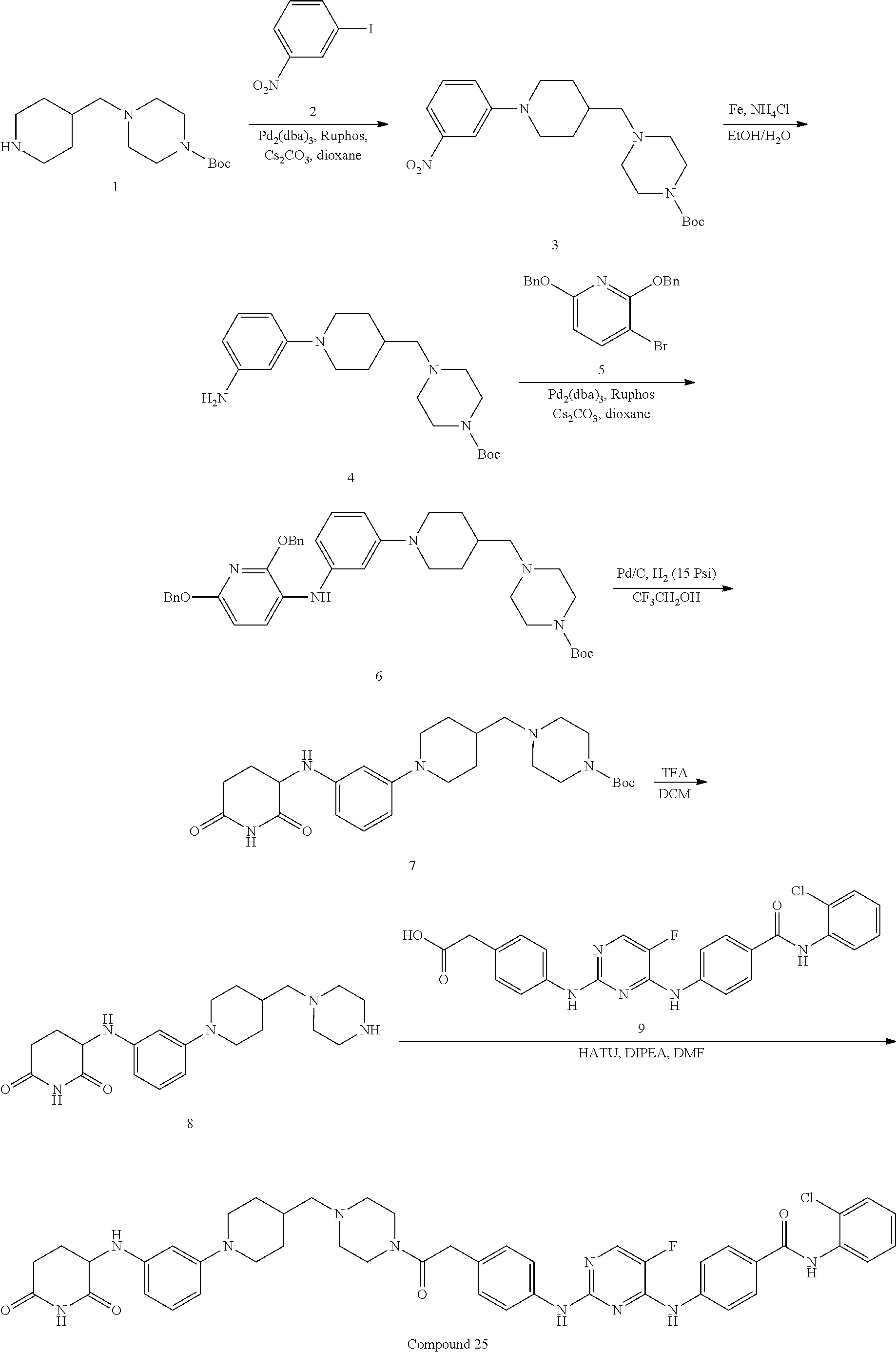

### Step 1. Synthesis of tert-butyl 4-((1-(3-nitrophenyl)piperidin-4-yl)methyl)piperazine-1-carboxylate (3)

A mixture of 1-iodo-3-nitrobenzene (1 g, 4.02 mmol) tert-butyl 4-(piperidin-4-ylmethyl)piperazine-1-carboxylate (1.66 g, 4.82 mmol, HOAc), Pd₂ (dba)₃ (367.75 mg, 401.60 mol), RuPhos (374.80 mg, 803.20 mol) and Cs₂CO₃ (6.54 g, 20.08 mmol) in dioxane (2 mL) was degassed and purged with N₂ for 3 times, and then the mixture was stirred at 100° C. for 16 hrs under N₂ atmosphere. LCMS showed tert-butyl 4-(piperidin-4-ylmethyl)piperazine-1-carboxylate was consumed completely and desired mass. The reaction mixture was filtered and concentrated under reduced pressure to give a residue, which was purified by flash silica gel chromatography (ISCO®; 20 g SepaFlash® Silica Flash Column, Eluent of 0~20% EtOAc/Petroleum ether, gradient @ 50 mL/min) to afford tert-butyl 4-((1-(3-nitrophenyl)piperidin-4-yl)methyl)piperazine-1-carboxylate (1.23 g, 2.86 mmol. 71.23% yield, 94.08% purity) as a yellow solid. MS(M+H)⁺=405.2.

### Step 2. Synthesis of tert-butyl 4-((1-(3-aminophenyl)piperidin-4-yl)methyl)piperazine-1-carboxylate (4)

To a solution of tert-butyl 4-((1-(3-nitrophenyl)piperidin-4-yl)methyl)piperazine-1-carboxylate (1.23 g, 2.86 mmol, 94.08% purity) in EtOH (10 mL) was added NH₄Cl (765.13 mg, 14.30 mmol) in H₂O (2 mL), followed by Fe (479.28 mg, 8.58 mmol). The resulting mixture was stirred at 80° C. for 1 hr. LCMS showed tert-butyl 4-((1-(3-nitrophenyl)piperidin-4-yl)methyl)piperazine-1-carboxylate was consumed completely and desired mass was. The reaction mixture was diluted with H₂O (40 mL) and extracted with EtOAc (20 mL×5). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0-80% EtOAc/Petroleum ether, gradient @ 50 mL/min) to afford tert-butyl 4-((1-(3-aminophenyl)piperidin-4-yl)methyl)piperazine-1-carboxylate (0.519 g, 1.33 mmol, 46.53% yield, 96.05% purity) as a yellow solid. MS(M+H)⁺=375.3.

### Step 3. Synthesis of tert-butyl 4-((1-(3-((2,6-bis(benzyloxy)pyridin-3-yl)amino)phenyl)piperidin-4-yl)methyl)piperazine-1-carboxylate (6)

A mixture of tert-butyl 4-((1-(3-aminophenyl)piperidin-4-yl)methyl)piperazine-1-carboxylate (0.4 g, 1.03 mmol, 96.05% purity), 2,6-bis(benzyloxy)-3-bromopyridine (455.77 mg, 1.23 mmol), Pd₂(dba)₃ (93.94 mg, 102.58 mol), RuPhos (95.74 mg, 205.17 μmol) and Cs₂CO₃ (1.00 g, 3.08 mmol) in dioxane (8 mL) was degassed and purged with N₂ for 3 times, and then the mixture was stirred at 100° C. for 14 hrs under N₂ atmosphere. LCMS showed tert-butyl 4-((1-(3-aminophenyl)piperidin-4-yl)methyl)piperazine-1-carboxylate was consumed completely and desired mass. The reaction mixture was filtered and the filtrate was concentrated under reduced pressure to give a residue, which was purified by flash silica gel chromatography (ISCO®; 20 g SepaFlash® Silica Flash Column, Eluent of 0-45% EtOAc/Petroleum ether, gradient @ 50 mL/min) to afford tert-butyl 4-((1-(3-((2,6-bis(benzyloxy)pyridin-3-yl)amino)phenyl)piperidin-4-yl)methyl)piperazine-1-carboxylate (0.519 g, 777.19 μmol, 75.76% yield, 99.41% purity) as a yellow solid. MS(M+H)⁺=664.5.

### Step 4. Synthesis of tert-butyl 4-((1-(3-((2,6-dioxopiperidin-3-yl)amino)phenyl)piperidin-4-yl)methyl)piperazine-1-carboxylate (7)

To a solution of tert-butyl 4-((1-(3-((2,6-bis(benzyloxy)pyridin-3-yl)amino)phenyl)piperidin-4-yl)methyl)piperazine-1-carboxylate (120 mg, 180.76 mol) in CF₃CH₂OH (3 mL) was added Pd/C (50 mg, 10% purity) under H₂ atmosphere. The suspension was degassed and purged with H₂ for 3 times. The mixture was stirred under H₂ (15 Psi) at 25° C. for 16 hr. LCMS showed tert-butyl 4-((1-(3-((2,6-bis(benzyloxy)pyridin-3-yl)amino)phenyl)piperidin-4-yl)methyl)piperazine-1-carboxylate was consumed completely and a main peak (89%) with desired mass. The reaction mixture was filtered and the filtrate was concentrated to afford tert-butyl 4-((1-(3-((2,6-dioxopiperidin-3-yl)amino)phenyl)piperidin-4-yl)methyl)piperazine-1-carboxylate (100 mg, 172.98 μmol, 95.69% yield, 84% purity) as yellow solid. MS(M+H)⁺=486.5.

### Step 5. Synthesis of 3-((3-(4-(piperazin-1-ylmethyl)piperidin-1-yl)phenyl)amino)piperidine-2,6-dione (8)

To a solution of tert-butyl 4-((1-(3-((2,6-dioxopiperidin-3-yl)amino)phenyl)piperidin-4-yl)methyl)piperazine-1-carboxylate (200.00 mg, 411.85 mol) in DCM (3 mL) was added TFA (1.54 g, 13.51 mmol, 1 mL). The mixture was stirred at 20° C. for 2 hrs. LCMS showed the starting material was consumed completely. The reaction mixture was concentrated under reduced pressure to afford 3-((3-(4-(piperazin-1-ylmethyl)piperidin-1-yl)phenyl)amino)piperidine-2,6-dione (0.2 g, 400.38 μmol, 97.22% yield, TFA) as a blue solid. The crude product was used for next step directly. MS(M+H)⁺=454.2.

### Step 6. Synthesis of N-(2-chlorophenyl)-4-((2-((4-(2-(4-((1-(3-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino)benzamide (Compound 25)

To a solution of 2-(4-((4-((4-((2-chlorophenyl)carbamoyl)phenyl)amino)-5-fluoropyrimidin-2-yl)amino)phenyl)acetic acid (246.18 mg, 400.38 μmol, 80% purity) and HATU (228.35 mg, 600.57 mol) in DMF (3 mL) was added DIPEA (310.48 mg, 2.40 mmol, 418.43 μL). The mixture was stirred at 20° C. for 0.5 hr. Then 3-((3-(4-(piperazin-1-ylmethyl)piperidin-1-yl)phenyl)amino)piperidine-2,6-dione (0.2 g, 400.38 μmol, TFA) was added and the resulting mixture was stirred at 20° C. for 16 hr. LCMS showed 3-((3-(4-(piperazin-1-ylmethyl)piperidin-1-yl)phenyl)amino)piperidine-2,6-dione was consumed completely and desired mass. The reaction mixture was poured into H₂O (10 mL) and extracted with EtOAc (10 mL×3). The combined organic layers were washed with brine, dried over Na₂SO₄, filtered and concentrated under reduced pressure to give a residue. The residue was purified by flash silica gel chromatography (ISCO®; 12 g SepaFlash® Silica Flash Column, Eluent of 0-100% EtOAc/Petroleum ether, gradient @ 50 mL/min), followed by prep-HPLC (column: Waters Xbridge 150*25 mm*5 um; mobile phase: [water (NH₄HCO₃)-ACN]; B %: 45%-75%, 8 min) and lyophilized to afford N-(2-chlorophenyl)-4-((2-((4-(2-(4-((1-(3-((2,6-dioxopiperidin-3-yl)amino)phenyl)piperidin-4-yl)methyl)piperazin-1-yl)-2-oxoethyl)phenyl)amino)-5-fluoropyrimidin-4-yl)amino) benzamide (5 mg, 5.64 μmol, 1.41% yield, 97% purity) as a white solid. MS(M+H)⁺=859.5.

¹H NMR (400 MHz, DMSO-d₆) δ=10.74 (s, 1H), 9.92 (s, 1H), 9.65 (s, 1H), 9.32 (s, 1H), 8.18 (d, J=3.5 Hz, 1H), 8.05-7.95 (m, 4H), 7.67-7.53 (m, 4H), 7.43-7.36 (m, 1H), 7.33-7.26 (m, 1H), 7.12 (d, J=8.4 Hz, 2H), 6.88 (t, J=8.0 Hz, 1H), 6.24 (s, 1H), 6.17 (br d, J=7.8 Hz, 1H), 6.12-6.07 (m, 1H), 5.61-5.56 (m, 1H), 4.34-4.25 (m, 1H), 3.64 (s, 2H), 3.60-3.51 (m, 3H), 3.49-3.42 (m, 5H), 2.61-2.57 (m, 2H), 2.56-2.55 (m, 1H), 2.27-2.18 (m, 4H), 2.14-2.10 (m, 2H), 1.90-1.79 (m, 1H), 1.76-1.65 (m, 2H), 1.64-1.51 (m, 1H), 1.20-1.10 (m, 2H).

### Example 26. Synthesis of N-(2-chlorophenyl)-4-((2-((4-(((1r,4r)-4-((4-(4-((2,6-dioxopiperidin-3-yl)amino)phenyl)piperazin-1-yl)methyl)cyclohexyl)carbamoyl)phenyl)amino)-5-fluoropyrimidin-4-yl)amino)benzamide (Compound 26)

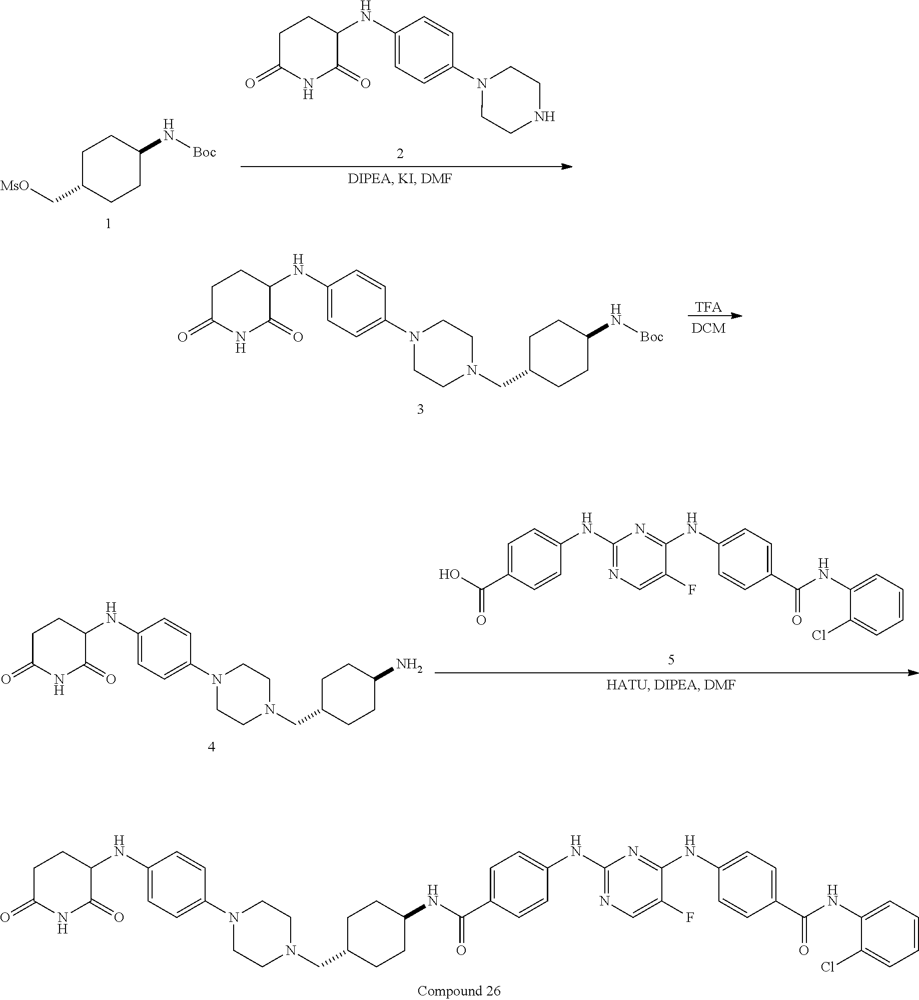

### Step 1. Synthesis of tert-butyl ((1r,4r)-4-((4-(4-((2,6-dioxopiperidin-3-yl)amino)phenyl)piperazin-1-yl)methyl)cyclohexyl)carbamate (3)

A mixture of 3-((4-(piperazin-1-yl)phenyl)amino)piperidine-2,6-dione (500 mg, 968.26 μmol, 2TFA salt), ((1r,4r)-4-((tert-butoxycarbonyl)amino)cyclohexyl)methyl methanesulfonate (208.35 mg, 677.78 mol), DIPEA (625.70 mg, 4.84 mmol, 843.27 μL) and KI (160.73 mg, 968.26 μmol) in DMF (7 mL) was stirred at 60° C. for 16 hours. LCMS showed 18% peak of 3-((4-(piperazin-1-yl)phenyl)amino)piperidine-2,6-dione remained and 72% peak with desired mass. Another portion of ((1r,4r)-4-((tert-butoxycarbonyl)amino)cyclohexyl)methyl methanesulfonate (59.53 mg, 193.65 mol) was added and the resulting mixture was stirred at 60° C. for 16 hours. LCMS showed 3-((4-(piperazin-1-yl)phenyl)amino)piperidine-2,6-dione was consumed completely and 92% peak with desired mass. The mixture was treated with AcOH to adjust pH<7 and purified by prep-HPLC (column: Phenomenex luna C18 150*25 mm*10 um; mobile phase: [water(TFA)-ACN]; gradient: 15%-45% B over 10 min). The eluent was lyophilized to afford tert-butyl ((1r,4r)-4-((4-(4-((2,6-dioxopiperidin-3-yl)amino)phenyl)piperazin-1-yl)methyl)cyclohexyl)carbamate (175 mg, 285.17 μmol, 29.45% yield, TFA salt) as a black solid. MS(M+H)⁺=500.3.

### Step 2. Synthesis of 3-((4-(4-(((1r,4r)-4-aminocyclohexyl)methyl)piperazin-1-yl)phenyl)amino)piperidine-2,6-dione (4)

A solution of tert-butyl ((1r,4r)-4-((4-(4-((2,6-dioxopiperidin-3-yl)amino)phenyl)piperazin-1-yl)methyl)cyclohexyl)carbamate (130 mg, 211.84 μmol, TFA salt) and TFA (1.54 g, 13.46 mmol, 1 mL) in DCM (4 mL) was stirred at 15° C. for 1 hour. LCMS showed the starting material was consumed completely. The mixture was concentrated in vacuum to afford 3-((4-(4-(((1r,4r)-4-aminocyclohexyl)methyl)piperazin-1-yl)phenyl)amino)piperidine-2,6-dione (135 mg, 2TFA salt) as a black gum, which was used directly. MS(M+H)⁺=400.2.

### Step 3. Synthesis of N-(2-chlorophenyl)-4-((2-((4-(((1r,4r)-4-((4-(4-((2,6-dioxopiperidin-3-yl)amino)phenyl)piperazin-1-yl)methyl)cyclohexyl)carbamoyl)phenyl)amino)-5-fluoropyrimidin-4-yl)amino)benzamide (Compound 26)

A mixture of 4-((4-((4-((2-chlorophenyl)carbamoyl)phenyl)amino)-5-fluoropyrimidin-2-yl)amino)benzoic acid (90 mg, 188.33 mol), HATU (93.09 mg, 244.83 μmol) and DIPEA (243.41 mg, 1.88 mmol) in DMF (4 mL) was stirred at 15° C. for 30 minutes. Then 3-((4-(4-(((1r,4r)-4-aminocyclohexyl)methyl)piperazin-1-yl)phenyl)amino)piperidine-2,6-dione (130.00 mg, 207.15 μmol, 2TFA salt) was added and the resulting mixture was stirred at 15° C. for 1 hour. LCMS showed trace of 3-((4-(4-(((1r,4r)-4-aminocyclohexyl)methyl)piperazin-1-yl)phenyl)amino)piperidine-2,6-dione remained and 60% peak with desired mass. The mixture was diluted with H₂O (30 mL) and extracted with a mixture EtOAc (15 mL×3). The combined organic layers were washed with brine (50 mL), dried over Na₂SO₄ and concentrated in vacuum. The residue was purified by prep-HPLC (column: Phenomenex luna C18 150*25 mm*10 um; mobile phase: [water(TFA)-ACN]; gradient: 26%-56% B over 9 min) and lyophilized to afford N-(2-chlorophenyl)-4-((2-((4-(((1r,4r)-4-((4-(4-((2,6-dioxopiperidin-3-yl)amino)phenyl)piperazin-1-yl)methyl)cyclohexyl)carbamoyl)phenyl)amino)-5-fluoropyrimidin-4-yl)amino)benzamide (24.0 mg, 15.95 μmol, 8.47% yield, 95% purity, 5TFA salt) as a brown solid. MS(M+H)⁺=859.2.

¹H NMR (400 MHz, DMSO-d₆) δ=10.82 (s, 1H), 9.94 (s, 1H), 9.77 (s, 1H), 9.63 (s, 1H), 9.25-9.15 (m, 1H), 8.25 (d, J=3.7 Hz, 1H), 8.16-7.97 (m, 6H), 7.85-7.79 (m, 4H), 7.65 (dd, J=1.3, 7.9 Hz, 1H), 7.57 (dd, J=1.3, 7.9 Hz, 1H), 7.46-7.36 (m, 1H), 7.35-7.23 (m, 1H), 6.83 (br d, J=8.8 Hz, 2H), 6.67 (d, J=8.9 Hz, 2H), 4.24 (dd, J=4.7, 11.2 Hz, 1H), 3.80-3.70 (m, 1H), 3.65-3.55 (m, 4H), 3.23-3.10 (m, 2H), 3.10-3.05 (m, 2H), 3.00-2.86 (m, 2H), 2.79-2.67 (m, 1H), 2.65-2.56 (m, 1H), 2.12-2.08 (m, 1H), 1.98-1.87 (m, 5H), 1.83-1.78 (m, 1H), 1.45-1.29 (m, 2H), 1.19-1.05 (m, 2H).

### Example 27. Synthesis of N-(2-chlorophenyl)-4-((2-((4-((1-(((1R,5S,6s)-3-(4-((2,6-dioxopiperidin-3-yl)amino)phenyl)-3-azabicyclo[3.1.0]hexan-6-yl)methyl)piperidin-4-yl)carbamoyl)phenyl)amino)-5-fluoropyrimidin-4-yl)amino)benzamide (Compound 27)

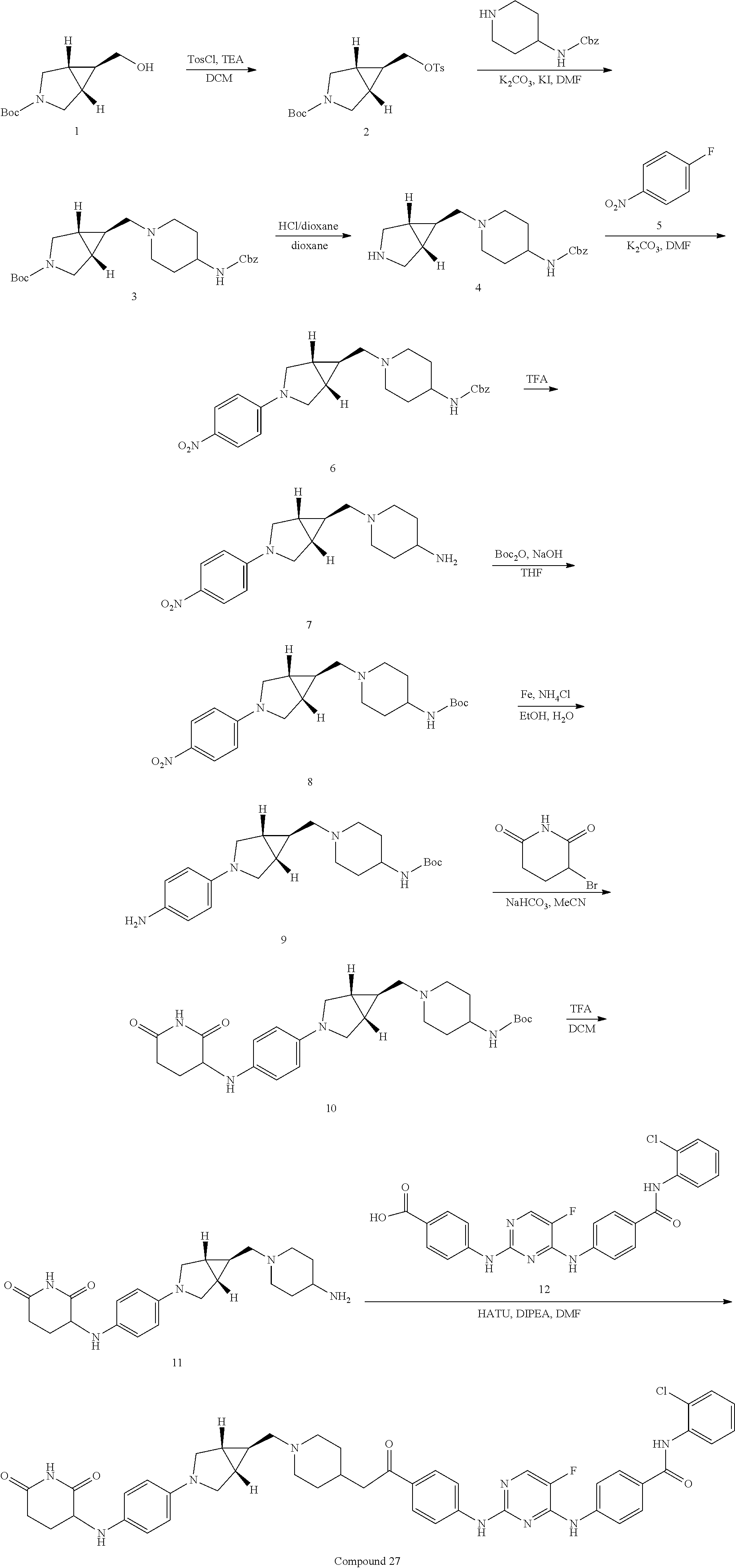

### Step 1. Synthesis of tert-butyl (1R,5S,6r)-6-((tosyloxy)methyl)-3-azabicyclo[3.1.0]hexane-3-carboxylate (2)

To a solution of tert-butyl (1R,5S,6r)-6-(hydroxymethyl)-3-azabicyclo[3.1.0]hexane-3-carboxylate (1 g, 4.69 mmol) in DCM (10 mL) were added TosCl (1.34 g, 7.03 mmol) and TEA (948.92 mg, 9.38 mmol, 1.31 mL) at 25° C. The mixture was stirred at 25° C. for 12 h. LCMS showed the starting material was consumed and the desired mass. The reaction mixture was diluted with water (30 mL) and extracted with DCM (15 mL×3). The combined organic layers were dried over Na₂SO₄, filtered and concentrated. The residue was purified by flash silica gel chromatography (12 g SepaFlash® Silica Flash Column, Eluent of 0~20% EtOAc/petroleum ether, gradient @ 100 mL/min) to afford tert-butyl (1R,5S,6r)-6-((tosyloxy)methyl)-3-azabicyclo[3.1.0]hexane-3-carboxylate (920 mg, 2.50 mmol, 53.40% yield) as a colorless oil. MS(M+H)⁺=368.1.

### Step 2. Synthesis of tert-butyl (1R,5S,6s)-6-((4-(((benzyloxy)carbonyl)amino)piperidin-1-yl)methyl)-3-azabicyclo[3.1.1]hexane-3-carboxylate (3)

To a solution of tert-butyl (1R,5S,6r)-6-((tosyloxy)methyl)-3-azabicyclo[3.1.0]hexane-3-carboxylate (920 mg, 2.50 mmol) in DMF (10 mL) were added K₂CO₃ (415.23 mg, 3.00 mmol), benzyl piperidin-4-ylcarbamate (645.26 mg, 2.75 mmol) and KI (41.56 mg, 250.37 mol) at 20° C. The resulting mixture was stirred at 80° C. for 4 h. LCMS showed 60% peak with desired mass. The reaction mixture was diluted with water (30 mL) and extracted with EtOAc (15 mL×3). The combined organic layers was washed with brine (15 mL×3), dried over Na₂SO₄, filtered. The filtrate was concentrated in vacuum and then purified by flash silica gel chromatography (12 g SepaFlash® Silica Flash Column, Eluent of 0~100% EtOAc/Petroleum ether to 0-10% MeOH in DCM, gradient @ 100 mL/min) to afford tert-butyl (1R,5S,6s)-6-((4-(((benzyloxy)carbonyl)amino)piperidin-1-yl)methyl)-3-azabicyclo[3.1.0]hexane-3-carboxylate (780 mg, 1.74 mmol, 69.63% yield, 96% purity) as a yellow oil. MS(M+H)⁺=430.4.

### Step 3. Synthesis of benzyl (1-(((1R,5S,6r)-3-azabicyclo[3.1.0]hexan-6-yl)methyl)piperidin-4-yl) carbamate (4)

To a solution of tert-butyl (1R,5S,6s)-6-((4-(((benzyloxy)carbonyl)amino)piperidin-1-yl)methyl)-3-azabicyclo[3.1.0]hexane-3-carboxylate (780 mg, 1.82 mmol) in dioxane (3 mL) was added HCl/dioxane (4 M, 9 mL) at 20° C. The mixture was stirred at 20° C. for 1 h. LCMS showed the starting material was consumed completely and 55% peak with desired mass. The reaction mixture was concentrated in vacuum to afford benzyl (1-(((1R,5S,6r)-3-azabicyclo[3.1.0]hexan-6-yl)methyl)piperidin-4-yl)carbamate (800 mg, crude, HCl) as a yellow oil. MS(M+H)⁺=330.2.

### Step 4. Synthesis of benzyl (1-(((1R,5S,6s)-3-(4-nitrophenyl)-3-azabicyclo[3.1.0]hexan-6-yl) methyl)piperidin-4-yl)carbamate (6)

To a solution of benzyl (1-(((1R,5S,6r)-3-azabicyclo[3.1.0]hexan-6-yl)methyl)piperidin-4-yl) carbamate (800 mg, 2.19 mmol, HCl) in DMF (10 mL) were added K₂CO₃ (604.35 mg, 4.37 mmol) and 1-fluoro-4-nitrobenzene (370.20 mg, 2.62 mmol) at 20° C. The mixture was stirred at 50° C. for 16 h. And then another portion of K₂CO₃ (906.52 mg, 6.56 mmol) was added and the reaction mixture was stirred at 50° C. for 19 h. LCMS showed the starting material was consumed completely and the desired mass. The reaction mixture was diluted with water (60 mL) and extracted with EtOAc (30 mL×3). The combined organic layer was washed with brine (40 mL), dried over Na₂SO₄, filtered. The filtrate was concentrated in vacuum to give a residue, which was purified by flash silica gel chromatography (12 g SepaFlash® Silica Flash Column, eluent of 0-10% MeOH in DCM, gradient@ 100 mL/min) to afford benzyl (1-(((1R,5S,6s)-3-(4-nitrophenyl)-3-azabicyclo[3.1.0]hexan-6-yl)methyl)piperidin-4-yl)carbamate (634 mg, 1.29 mmol, 59.21% yield, 92% purity) as a yellow solid. MS(M+H)⁺=451.2.

### Step 5. Synthesis of 1-(((1R,5S,6s)-3-(4-nitrophenyl)-3-azabicyclo[3.1.0]hexan-6-yl)methyl)piperidin-4-amine (7)

A solution of benzyl (1-(((1R,5S,6s)-3-(4-nitrophenyl)-3-azabicyclo[3.1.0]hexan-6-yl)methyl)piperidin-4-yl)carbamate (580 mg, 1.29 mmol) in TFA (6 mL) was stirred at 40° C. for 5 h. LCMS showed 32% the desired mass. The mixture was concentrated under reduced pressure to afford 1-(((1R,5S,6s)-3-(4-nitrophenyl)-3-azabicyclo[3.1.0]hexan-6-yl)methyl)piperidin-4-amine (0.6 g, crude, TFA) as a yellow oil. MS(M+H)⁺=317.1.

### Step 6. Synthesis of tert-butyl (1-(((1R,5S,6s)-3-(4-nitrophenyl)-3-azabicyclo[3.1.0]hexan-6-yl) methyl)piperidin-4-yl)carbamate (8)

To a solution of 1-(((1R,5S,6s)-3-(4-nitrophenyl)-3-azabicyclo[3.1.0]hexan-6-yl)methyl)piperidin-4-amine (0.6 g, 1.39 mmol, TFA) in THF (10 mL) was added NaOH (1 M, 11.15 mL) and Boc₂O (608.47 mg, 2.79 mmol, 640.49 μL) and the mixture (pH=8) was stirred at 20° C. for 14 h. NaOH (1 M, 0.7 mL) was added and the mixture was stirred at 20° C. for 18 h. The mixture was extracted with EtOAc (10 mL×2), the combined organic layer was washed with water (10 mL), dried over Na₂SO₄ and filtered. The filtrate was concentrated under reduced pressure. The residue was purified by flash silica gel chromatography (5 g SepaFlash® Silica Flash Column, Eluent of 80-100% EtOAc/Petroleum ether to 10-30% MeOH/EtOAc gradient @ 50 mL/min) and then purified by prep-HPLC (column: Phenomenex Luna C18 150*25 mm*10 um; mobile phase: [water (FA)-ACN]; gradient: 20%-50% B over 9 min). The eluent was adjusted the pH=9 with saturation NaHCO₃ and then extracted with EtOAc (20 mL×2), the combined organic layer was washed with water (10 mL), dried over Na₂SO₄ and filtered. The filtrate was concentrated under reduced pressure to afford tert-butyl (1-(((1R,5S,6s)-3-(4-nitrophenyl)-3-azabicyclo[3.1.0]hexan-6-yl)methyl)piperidin-4-yl)carbamate (170 mg, 408.15 μmol, 29.28% yield) as a yellow solid. MS(M+H)⁺=417.2.

### Step 7. Synthesis of tert-butyl (1-(((1R,5S,6s)-3-(4-aminophenyl)-3-azabicyclo[3.1.0]hexan-6-yl) methyl)piperidin-4-yl)carbamate (9)

To a solution of tert-butyl (1-(((1R,5S,6s)-3-(4-nitrophenyl)-3-azabicyclo[3.1.0]hexan-6-yl) methyl)piperidin-4-yl)carbamate (150 mg, 360.13 mol) and NH₄Cl (97.50 mg, 1.82 mmol) in EtOH (7 mL) and H₂O (3 mL) was added Fe (105.00 mg, 1.88 mmol) and the mixture was stirred at 80° C. for 1 h. LCMS showed 89% of the desired mass. The mixture was filtered and the filter cake was washed with EtOH (20 mL) and THF (20 mL). The filtrate was concentrated under reduced pressure. The crude was diluted with EtOAc (10 mL) and water (10 mL) and then the mixture was adjusted the pH=9 with saturation Na₂CO₃. The mixture was extracted with EtOAc (10 mL×3), the combined organic layers was washed with water (10 mL), dried over Na₂SO₄, filtered and concentrated under reduced pressure to afford tert-butyl (1-(((1R,5S,6s)-3-(4-aminophenyl)-3-azabicyclo[3.1.0]hexan-6-yl)methyl)piperidin-4-yl)carbamate (140 mg, crude) as a yellow solid. MS(M+H)⁺=387.3.

### Step 8. Synthesis of tert-butyl (1-(((1R,5S,6s)-3-(4-((2,6-dioxopiperidin-3-yl)amino)phenyl)-3-azabicyclo[3.1.0]hexan-6-yl)methyl)piperidin-4-yl)carbamate (10)

To a solution of tert-butyl (1-(((1R,5S,6s)-3-(4-aminophenyl)-3-azabicyclo[3.1.0]hexan-6-yl) methyl)piperidin-4-yl)carbamate (120 mg, 310.45 mol) and 3-bromopiperidine-2,6-dione (90 mg, 468.72 μmol) in MeCN (1.5 mL) was added NaHCO₃ (79 mg, 940.40 μmol, 36.59 μL) and the mixture was stirred at 80° C. for 14 h. LCMS showed 49% of the desired mass. The mixture was concentrated under reduced pressure. The residue was purified by flash silica gel chromatography (5 g SepaFlash® Silica Flash Column, eluent of 10% MeOH in DCM, gradient @ 30 mL/min) to afford tert-butyl (1-(((1R,5S,6s)-3-(4-((2,6-dioxopiperidin-3-yl)amino)phenyl)-3-azabicyclo[3.1.0]hexan-6-yl)methyl)piperidin-4-yl)carbamate (130 mg, 261.24 μmol, 84.15% yield, 100% purity) as a yellow solid. MS(M+H)⁺=498.3.

### Step 9. Synthesis of 3-((4-((1R,5S,6s)-6-((4-aminopiperidin-1-yl)methyl)-3-azabicyclo[3.1.0] hexan-3-yl)phenyl)amino)piperidine-2,6-dione (11)

To a solution of tert-butyl (1-(((1R,5S,6s)-3-(4-((2,6-dioxopiperidin-3-yl)amino)phenyl)-3-azabicyclo[3.1.0]hexan-6-yl)methyl)piperidin-4-yl)carbamate (130 mg, 261.24 mol) in DCM (1.3 mL) was added TFA (460.50 mg, 4.04 mmol, 0.3 mL) at 0° C. and the mixture was stirred at 0° C. for 1 h. LCMS showed 78% of the desired mass. The mixture was concentrated under reduced pressure to afford 3-((4-((1R,5S,6s)-6-((4-aminopiperidin-1-yl)methyl)-3-azabicyclo[3.1.0]hexan-3-yl)phenyl)amino)piperidine-2,6-dione (140 mg, crude, TFA) as a blue oil. MS(M+H)⁺=398.4.

### Step 10. Synthesis of N-(2-chlorophenyl)-4-((2-((4-((1-(((1R,5S,6s)-3-(4-((2,6-dioxopiperidin-3-yl)amino)phen yl)-3-azabicyclo[3.1.0]hexan-6-yl)methyl)piperidin-4-yl)carbamoyl)phenyl)amino)-5-fluoropyrimidin-4-yl)amino)benzamide (Compound 27)

To a solution of 4-((4-((4-((2-chlorophenyl)carbamoyl)phenyl)amino)-5-fluoropyrimidin-2-yl)amino)benzoic acid (120 mg, 251.11 μmol) and HATU (114.58 mg, 301.33 mol) in DMF (1.5 mL) was added DIPEA (445.20 mg, 3.44 mmol, 0.6 mL) and the mixture was stirred at 20° C. for 15 min. Then a solution of 3-((4-((1R,5S,6s)-6-((4-aminopiperidin-1-yl)methyl)-3-azabicyclo[3.1.0]hexan-3-yl)phenyl)amino)piperidine-2,6-dione (140 mg, 273.69 μmol, TFA) in DMF (1.5 mL) was added and the mixture was stirred at 20° C. for 1 h. LCMS showed 66% of the desired mass The mixture was diluted with water (50 mL) and EtOAc (10 mL) then filtered. The filter cake was washed with water (10 mL) and EtOAc (10 mL). The filter cake was collected and the solvent was removed under reduced pressure to afford a crude product (60 mg, 73% purity). The filtrate was extracted with EtOAc (10 mL×2). The combined organic layer was washed with water (20 mL), dried over Na₂ SO₄ and filtered. The filtrate was concentrated under reduced pressure to afford another part of the crude product (140 mg, 67% purity). The combined crude product was purified by prep-HPLC (column: Phenomenex Luna C18 150*25 mm*10 um; mobile phase: [water (TFA)-ACN]; gradient: 20%-50% B over 9 min) and the eluent was lyophilized to afford the product (90 mg, 91% purity, 3TFA), which was further purified by prep-HPLC (column: Waters Xbridge 150*25 mm*5 um; mobile phase: [water (NH₄HCO₃)-ACN]; gradient: 27%-57% B over min) and lyophilized to afford N-(2-chlorophenyl)-4-((2-((4-((1-(((1R,5S,6s)-3-(4-((2,6-dioxopiperidin-3-yl)amino)phenyl)-3-azabicyclo[3.1.0]hexan-6-yl)methyl)piperidin-4-yl)carbamoyl)phenyl)amino)-5-fluoropyrimidin-4-yl)amino)benzamide (13.3 mg, 14.43 μmol, 5.75% yield, 93% purity) as a red solid. MS(M+H)⁺=857.3.

¹H NMR (400 MHz, DMSO-d₆) δ=10.74 (br s, 1H), 9.93 (s, 1H), 9.74 (s, 1H), 9.61 (s, 1H), 8.25 (d, J=3.5 Hz, 1H), 8.07-7.98 (m, 5H), 7.80-7.75 (m, 4H), 7.67-6.63 (m, 1H), 7.58-7.54 (m, 1H), 7.42-7.37 (m, 1H), 7.32-7.25 (m, 1H), 6.60 (d, J=8.8 Hz, 2H), 6.41 (br d, J=8.8 Hz, 2H), 5.10 (d, J=7.1 Hz, 1H), 4.16-4.06 (m, 1H), 3.80-3.67 (m, 1H), 3.47 (br d, J=8.9 Hz, 2H), 3.02-2.90 (m, 4H), 2.75-2.68 (m, 1H), 2.61-2.54 (m, 2H), 2.27-2.22 (m, 2H), 2.15-2.07 (m, 1H), 2.04-1.94 (m, 2H), 1.85-1.72 (m, 3H), 1.62-1.52 (m, 2H), 1.48-1.44 (m, 2H), 0.87-0.79 (m, 1H).

### Example 28. Synthesis of N-(2-chlorophenyl)-4-((2-((4-(4-(3-(4-((2,6-dioxopiperidin-3-yl)amino)phenyl)-3,6-di azabicyclo[3.2.1]octan-6-yl)piperidine-1-carbonyl)phenyl)amino)-5-fluoropyrimidin-4-yl)amino)benzamide (Compound 28)

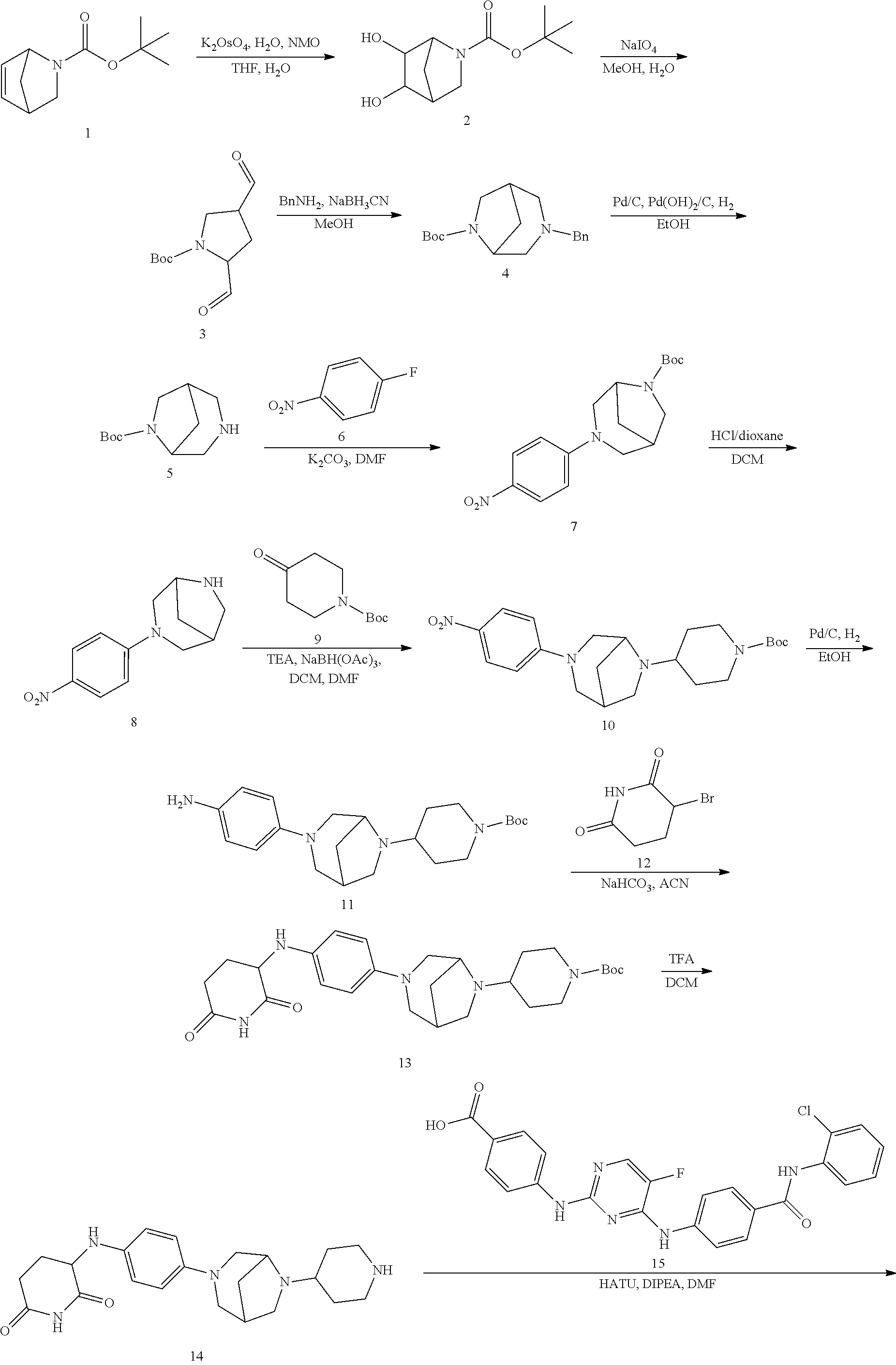

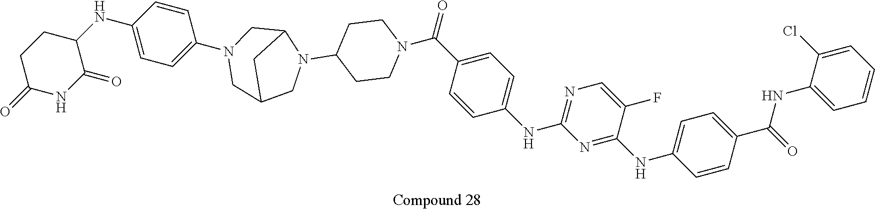 [SMILES: O=C1CCC(Nc2ccc(N3CC4CC(C3)N(C3CCN(C(=O)c5ccc(Nc6ncc(F)c(Nc7ccc(C(=O)Nc8ccccc8Cl)cc7)n6)cc5)CC3)C4)cc2)C(=O)N1]

### Step 1. Synthesis of tert-butyl 5,6-dihydroxy-2-azabicyclo[2.2.1]heptane-2-carboxylate (2)

To a solution of tert-butyl 2-azabicyclo[2.2.1]hept-5-ene-2-carboxylate (1 g, 5.12 mmol) and K₂OsO₄·2H₂O (94 mg, 255.12 μmol) in THF (10 mL) and H₂O (2.5 mL) was added NMO (720 mg, 6.15 mmol) and the mixture was stirred at 20° C. for 14 h. LCMS showed 58% of the desired mass. The mixture was quenched with saturated NaHSO₃ solution (20 mL) and extracted with EtOAc (10 mL×3). The combined organic layers were dried over Na₂SO₄ and filtered. The filtrate was concentrated under reduced pressure and the residue was purified by flash silica gel chromatography (12 g SepaFlash® Silica Flash Column, eluent of 30% EtOAc in petroleum ether, gradient @ 80 mL/min) to afford tert-butyl 5,6-dihydroxy-2-azabicyclo[2.2.1]heptane-2-carboxylate (660 mg, 2.88 mmol, 56.21% yield) as a yellow oil. MS(M+H)⁺=230.2.

### Step 2. Synthesis of tert-butyl 2,4-diformylpyrrolidine-1-carboxylate (3)

To a solution of tert-butyl 5,6-dihydroxy-2-azabicyclo[2.2.1]heptane-2-carboxylate (560 mg, 2.44 mmol) in H₂O (2.5 mL) and MeOH (10 mL) was slowly added NaIO₄ (784.00 mg, 3.67 mmol, 203.11 μL) and the mixture was stirred at 20° C. for 2 h. LCMS showed the starting material was consumed completely. The mixture was filtered and the filter cake was washed with EtOAc (20 mL). The filtrate was quenched with saturated Na₂SO₃ solution (20 mL) and then extracted with EtOAc (10 mL×3). The combined organic layers were washed with Na₂SO₃ solution (10 mL×2), dried over Na₂SO₄, filtered and concentrated in vacuum to afford tert-butyl 2,4-diformylpyrrolidine-1-carboxylate (510 mg, crude) as a yellow oil. MS(M+H)⁺=228.2.

### Step 3. Synthesis of tert-butyl 3-benzyl-3,6-diazabicyclo[3.2.1]octane-6-carboxylate (4)

A solution of tert-butyl 2,4-diformylpyrrolidine-1-carboxylate (510 mg, 2.24 mmol) and BnNH₂ (235.92 mg, 2.20 mmol) in MeOH (10 mL) was stirred at 20° C. for 10 min, followed by the addition of NaBH₃CN (423.08 mg, 6.73 mmol). The resulting mixture was stirred at 20° C. for 14 h. LCMS showed 72% peak of the desired mass. The mixture was concentrated under reduced pressure. The residue was purified by flash silica gel chromatography (5 g SepaFlash® Silica Flash Column, eluent of 80-100% EtOAc in petroleum ether, gradient @ 50 mL/min) to afford tert-butyl 3-benzyl-3,6-diazabicyclo[3.2.1]octane-6-carboxylate (450 mg, 1.49 mmol, 66.31% yield) as a yellow oil. MS(M+H)⁺=303.3.

### Step 4. Synthesis of tert-butyl 3,6-diazabicyclo[3.2.1]octane-6-carboxylate (5)

To a solution of tert-butyl 3-benzyl-3,6-diazabicyclo[3.2.1]octane-6-carboxylate (450 mg, 1.49 mmol) in EtOH (10 mL) was slowly added Pd/C (50 mg, 10% purity) and Pd(OH)₂/C (50 mg, 20% purity) under N₂ atmosphere and the mixture was stirred at 60° C. under H₂ (45 Psi) atmosphere for 14 h. LCMS showed trace of the starting material remained and the desired mass was detected. The mixture was filtered and the filter cake was washed with EtOH (20 mL) and THF (20 mL). The filtrate was concentrated under reduced pressure to afford tert-butyl 3,6-diazabicyclo[3.2.1]octane-6-carboxylate (330 mg, crude) as a white solid. The crude product was used for the next step directly.

### Step 5. Synthesis of tert-butyl 3-(4-nitrophenyl)-3,6-diazabicyclo[3.2.1]octane-6-carboxylate (7)

To a solution of 1-fluoro-4-nitrobenzene (250 mg, 1.77 mmol, 187.97 μL) and tert-butyl 3,6-diazabicyclo[3.2.1]octane-6-carboxylate (330 mg, 1.55 mmol) in DMF (6 mL) was added K₂CO₃ (644.52 mg, 4.66 mmol) and the mixture was stirred at 40° C. for 14 h. LCMS showed 65% peak of the desired mass. The mixture was diluted with water (30 mL) and then extracted with EtOAc (10 mL×3). The combined organic layers was washed with brine (10 mL×3), dried over Na₂SO₄ and filtered. The filtrate was concentrated under reduced pressure. The residue was purified by flash silica gel chromatography (5 g SepaFlash® Silica Flash Column, eluent of 30~50% EtOAc in petroleum ether, gradient @ 50 mL/min) to afford tert-butyl 3-(4-nitrophenyl)-3,6-diazabicyclo[3.2.1]octane-6-carboxylate (440 mg, 1.32 mmol, 84.90% yield, 100% purity) as a yellow solid. MS(M+Na)⁺=356.2.

### Step 6. Synthesis of 3-(4-nitrophenyl)-3,6-diazabicyclo[3.2.1]octane (8)

To a solution of tert-butyl 3-(4-nitrophenyl)-3,6-diazabicyclo[3.2.1]octane-6-carboxylate (440 mg, 1.32 mmol) in DCM (8 mL) was added HCl/dioxane (4 M, 8 mL) and the mixture was stirred at 20° C. for 1 h. LCMS showed a main peak with the desired mass. The mixture was concentrated under reduced pressure to afford 3-(4-nitrophenyl)-3,6-diazabicyclo[3.2.1]octane (350 mg, crude, HCl) as a yellow solid. MS(M+H)⁺=234.2.

### Step 7. Synthesis of tert-butyl 4-(3-(4-nitrophenyl)-3,6-diazabicyclo[3.2.1]octan-6-yl)piperidine-1-carboxylate (10)

To a solution of 3-(4-nitrophenyl)-3,6-diazabicyclo[3.2.1]octane (350 mg, 1.30 mmol, HCl) in DCM (10 mL) and DMF (4 mL) were added TEA (392.58 mg, 3.88 mmol, 540 μL), 4 Å MS (100 mg) and tert-butyl 4-oxopiperidine-1-carboxylate (388 mg, 1.95 mmol). After stirring at 20° C. for 30 min, NaBH(OAc)₃ (825.05 mg, 3.89 mmol) was added and the resulting mixture was stirred at 20° C. for 14 h. LCMS showed a main peak with the desired mass. The mixture was filtered and the filter cake was washed with EtOAc (50 mL). The filtrate was concentrated under reduced pressure. The residue was diluted with water (10 mL) and adjusted the pH=9 with saturated Na₂CO₃ solution, followed by the extraction with EtOAc (10 mL×3). The combined organic layers was washed with brine (10 mL×2), dried over Na₂SO₄ and filtered. The filtrate was concentrated under reduced pressure. The residue was purified by flash silica gel chromatography (12 g SepaFlash® Silica Flash Column, eluent of 5-30% EtOH in EtOAc, gradient @ 100 mL/min) to afford tert-butyl 4-(3-(4-nitrophenyl)-3,6-diazabicyclo[3.2.1]octan-6-yl)piperidine-1-carboxylate (0.5 g, 1.18 mmol, 90.66% yield, 98% purity) as a yellow oil. MS(M+H)⁺=417.2.

### Step 8. Synthesis of tert-butyl 4-(3-(4-aminophenyl)-3,6-diazabicyclo[3.2.1]octan-6-yl)piperidine-1-carboxylate (11)

To a solution of tert-butyl 4-(3-(4-nitrophenyl)-3,6-diazabicyclo[3.2.1]octan-6-yl)piperidine-1-carboxylate (0.5 g, 1.20 mmol) in EtOH (10 mL) was slowly added Pd/C (0.1 g, 10% purity) under N₂ and the mixture was stirred at 25° C. under H₂ atmosphere (45 Psi) for 14 h. LCMS showed 78% peak of the desired mass. The mixture was filtered and the filter cake was washed with EtOH (20 mL) and THF (50 mL). The filtrate was concentrated under reduced pressure to afford tert-butyl 4-(3-(4-aminophenyl)-3,6-diazabicyclo[3.2.1]octan-6-yl)piperidine-1-carboxylate (450 mg, crude) as a yellow solid. MS(M+H)⁺=387.3.

### Step 9. Synthesis of tert-butyl 4-(3-(4-((2,6-dioxopiperidin-3-yl)amino)phenyl)-3,6-diazabicyclo[3.2.1]octan-6-yl)piperidine-1-carboxylate (13)

To a solution of tert-butyl 4-(3-(4-aminophenyl)-3,6-diazabicyclo[3.2.1]octan-6-yl)piperidine-1-carboxylate (400 mg, 1.03 mmol) and 3-bromopiperidine-2,6-dione (328.00 mg, 1.71 mmol) in ACN (4 mL) was added NaHCO₃ (304.00 mg, 3.62 mmol) and the mixture was stirred at 80° C. for 14 h. LCMS showed the desired mass. The mixture was filtered and washed with EtOAc (10 mL) and THF (20 mL). The filtrate was concentrated under reduced pressure. The residue was purified by flash silica gel chromatography (5 g SepaFlash® Silica Flash Column, eluent of 20-30% MeOH in EtOAc, gradient @ 50 mL/min) to afford tert-butyl 4-(3-(4-((2,6-dioxopiperidin-3-yl)amino)phenyl)-3,6-diazabicyclo[3.2.1]octan-6-yl)piperidine-1-carboxylate (0.4 g, 803.81 μmol, 77.67% yield, 100% purity) as a yellow solid. MS(M+H)⁺=498.3.

### Step 10. Synthesis of 3-((4-(6-(piperidin-4-yl)-3,6-diazabicyclo[3.2.1]octan-3-yl)phenyl)amino)piperidine-2, 6-dione (14)

To a solution of tert-butyl 4-(3-(4-((2,6-dioxopiperidin-3-yl)amino)phenyl)-3,6-diazabicyclo[3.2.1]octan-6-yl)piperidine-1-carboxylate (0.2 g, 401.91 mol) in DCM (2 mL) was added TFA (706.10 mg, 6.19 mmol, 460 μL) and the mixture was stirred at 20° C. for 1 h. LCMS showed the desired mass and the starting material was consumed. The mixture was concentrated under reduced pressure to afford 3-((4-(6-(piperidin-4-yl)-3,6-diazabicyclo[3.2.1]octan-3-yl)phenyl)amino)piperidine-2, 6-dione (0.2 g, crude, TFA) as a blue oil.

### Step 11. Synthesis of N-(2-chlorophenyl)-4-((2-((4-(4-(3-(4-((2,6-dioxopiperidin-3-yl)amino)phenyl)-3,6-diaza bicyclo[3.2.1]octan-6-yl)piperidine-1-carbonyl)phenyl)amino)-5-fluoropyrimidin-4-yl) amino)benzamide (Compound 28)

To a solution of 4-((4-((4-((2-chlorophenyl)carbamoyl)phenyl)amino)-5-fluoropyrimidin-2-yl) amino)benzoic acid (160 mg, 334.82 μmol) and HATU (153 mg, 402.39 μmol) in DMF (3 mL) was added DIPEA (86.55 mg, 669.63 μmol, 116.64 μL) and the mixture was stirred at 20° C. for 15 min. Then a solution of 3-((4-(6-(piperidin-4-yl)-3,6-diazabicyclo[3.2.1]octan-3-yl)phenyl)amino)piperidine-2, 6-dione (0.2 g, 390.98 μmol, TFA) and DIPEA (445.20 mg, 3.44 mmol, 0.6 mL) in DMF (3 mL) (0° C.) was added and the mixture was stirred at 20° C. for 14 h. LCMS showed 66% peak of the desired mass. The mixture was diluted with water (10 mL) and then filtered. The filter cake was washed with water (10 mL) and purified by prep-HPLC (column: Phenomenex luna C18 150*40 mm*15 um; mobile phase: [water (TFA)-ACN]; gradient: 15%-45% B over 10 min). The crude product was diluted with MeCN (3 mL), water (10 mL) and DCM (10 mL). The mixture was adjusted pH=8 with saturated NaHCO₃ solution and the mixture was extracted with DCM (10 mL×3). The combined organic layer was washed with water (10 mL), dried over Na₂SO₄ and filtered. The filtrate was concentrated under reduced pressure. The product was purified by prep-TLC (SiO2, DCM:MeOH=10:1). The product was further purified by prep-HPLC (column: Waters Xbridge 150*25 mm*5 um; mobile phase: [water (NH₄HCO₃)-ACN]; gradient: 27%-57% B over 9 min) and lyophilized to afford N-(2-chlorophenyl)-4-((2-((4-(4-(3-(4-((2,6-dioxopiperidin-3-yl)amino)phenyl)-3,6-diaza bicyclo[3.2.1]octan-6-yl)piperidine-1-carbonyl)phenyl)amino)-5-fluoropyrimidin-4-yl) amino)benzamide (24.6 mg, 26.40 μmol, 50.29% yield, 92% purity) as a red solid. MS(M+H)⁺=857.4.

¹H NMR (400 MHz, DMSO-d₆) δ=10.74 (s, 1H), 9.91 (s, 1H), 9.72 (s, 1H), 9.54 (s, 1H), 8.24-8.21 (m, 1H), 8.04-7.96 (m, 4H), 7.73 (br d, J=8.2 Hz, 2H), 7.65 (d, J=7.6 Hz, 1H), 7.53 (d, J=8.3 Hz, 1H), 7.40-7.34 (m, 1H), 7.33-7.25 (m, 3H), 6.69-6.57 (m, 4H), 5.22 (br d, J=7.2 Hz, 1H), 4.20-4.11 (m, 1H), 3.60-3.52 (m, 1H), 3.33-3.32 (m, 4H), 3.16-2.98 (m, 3H), 2.81-2.57 (m, 6H), 2.43-2.31 (m, 1H), 2.16-2.05 (m, 1H), 1.98-1.75 (m, 4H), 1.52-1.29 (m, 3H).

### Example 29. Synthesis of N-(2-chlorophenyl)-4-((2-((4-(4-((3-(4-((2,6-dioxopiperidin-3-yl)amino)phenyl)-3,6-diazabicyclo[3.2.1]octan-6-yl)methyl)piperidine-1-carbonyl)phenyl)amino)-5-fluoro pyrimidin-4-yl)amino)benzamide (Compound 29)

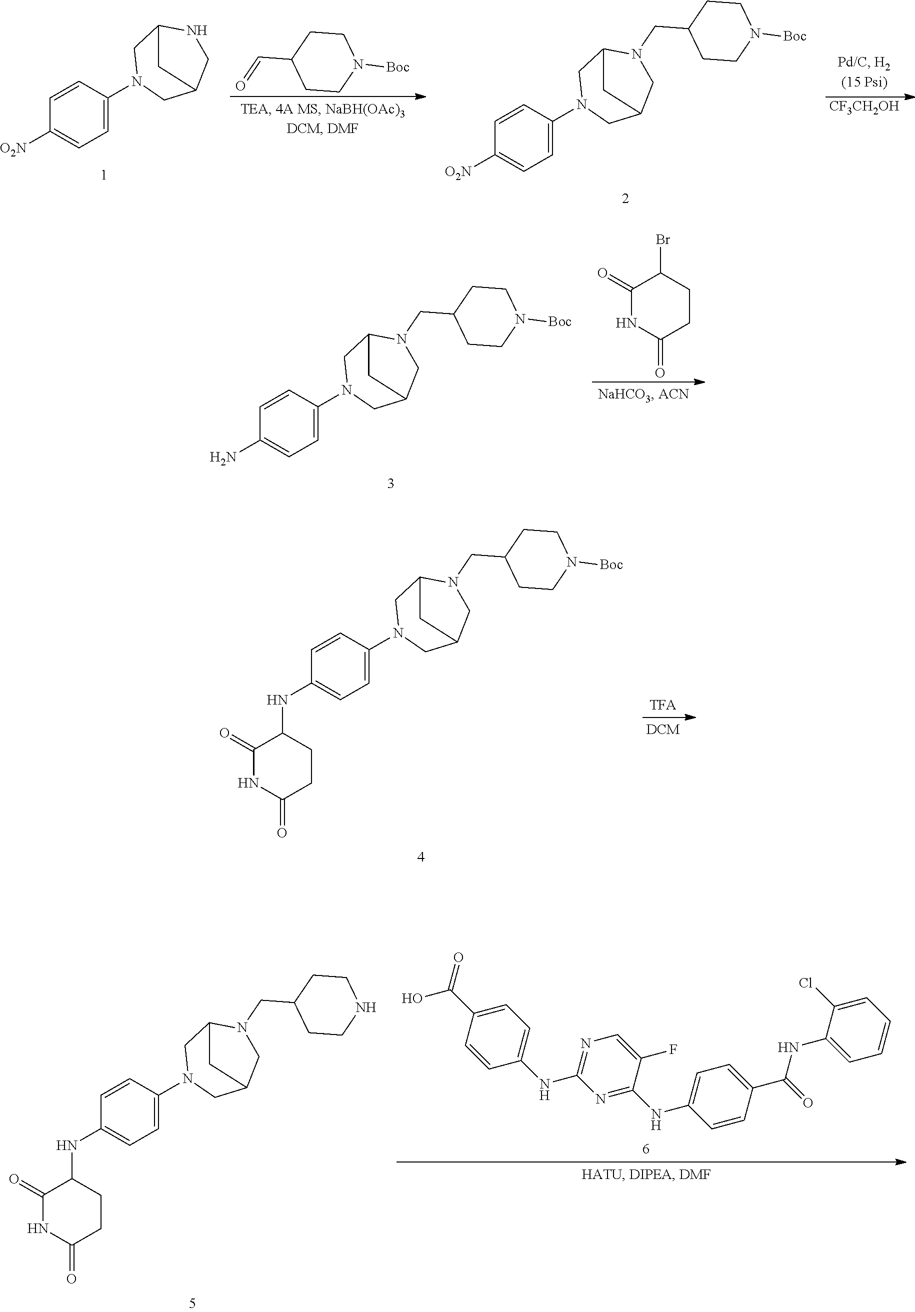

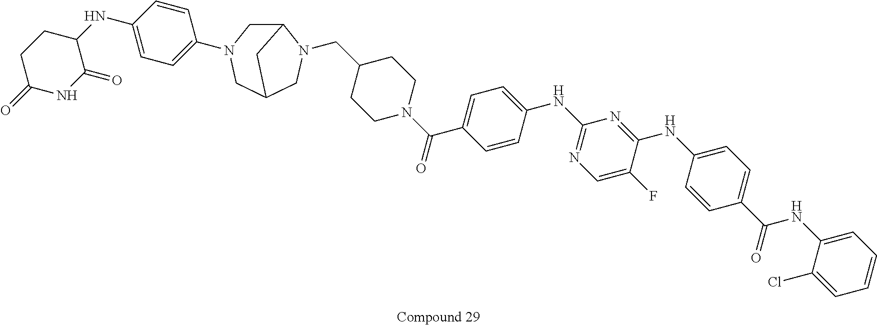 [SMILES: O=C1CCC(Nc2ccc(N3CC4CC(C3)N(CC3CCN(C(=O)c5ccc(Nc6ncc(F)c(Nc7ccc(C(=O)Nc8ccccc8Cl)cc7)n6)cc5)CC3)C4)cc2)C(=O)N1]

### Step 1. Synthesis of tert-butyl 4-((3-(4-nitrophenyl)-3,6-diazabicyclo[3.2.1]octan-6-yl)methyl)piperidine-1-carboxylate (2)

To a solution of 3-(4-nitrophenyl)-3,6-diazabicyclo[3.2.1]octane (160 mg, 593.19 mol, HCl) in DCM (3 mL) and DMF (3 mL) was added TEA (305.34 mg, 3.02 mmol) and 4 Å MS (50 mg) at 0° C., followed by the addition of tert-butyl 4-formylpiperidine-1-carboxylate (189 mg, 886.19 μmol). After stirring at 20° C. for 0.5 h, NaBH(OAc)₃ (377 mg, 1.78 mmol) was added. The resulting mixture was stirred at 20° C. for 14 h. LCMS showed a main peak with the desired mass. The mixture was filtered and washed with EtOAc (10 mL). The filtrate was concentrated under reduced pressure. The crude was diluted with water (10 mL) and adjusted the pH=9 with saturated Na₂CO₃ solution. And then the mixture was extracted with EtOAc (10 mL×3). The combined organic layer was washed with brine (10 mL×2), dried over Na₂SO₄ and filtered. The filtrate was concentrated under reduced pressure and then purified by flash silica gel chromatography (5 g SepaFlash® Silica Flash Column, eluent of 0~10% EtOH in EtOAc, gradient @ 50 mL/min) to afford tert-butyl 4-((3-(4-nitrophenyl)-3,6-diazabicyclo[3.2.1]octan-6-yl)methyl)piperidine-1-carboxylate (160 mg, 364.19 μmol, 61.40% yield, 98% purity) as a yellow oil. MS(M+H)⁺=431.2.

### Step 2. Synthesis of tert-butyl 4-((3-(4-aminophenyl)-3,6-diazabicyclo[3.2.1]octan-6-yl)methyl)piperidine-1-carboxylate (3)

To a mixture of Pd/C (50 mg, 10% purity) in CF₃CH₂OH (5 mL) was added a solution of tert-butyl 4-((3-(4-nitrophenyl)-3,6-diazabicyclo[3.2.1]octan-6-yl)methyl)piperidine-1-carboxylate (160 mg, 371.63 mol) in CF₃CH₂OH (5 mL) under N₂ atmosphere. The mixture was stirred at 25° C. under H₂ atmosphere (45 Psi) for 14 h. LCMS showed the desired mass. The mixture was filtered and washed with THF (100 mL). The filtrate was concentrated under reduced pressure to afford tert-butyl 4-((3-(4-aminophenyl)-3,6-diazabicyclo[3.2.1]octan-6-yl)methyl)piperidine-1-carboxylate (140 mg, crude) as a yellow solid. MS(M+H)⁺=401.4.

### Step 3. Synthesis of tert-butyl 4-((3-(4-((2,6-dioxopiperidin-3-yl)amino)phenyl)-3,6-diazabicyclo[3.2.1]octan-6-yl)methyl)piperidine-1-carboxylate (4)

To a solution of tert-butyl 4-((3-(4-aminophenyl)-3,6-diazabicyclo[3.2.1]octan-6-yl)methyl)piperidine-1-carboxylate (140 mg, 349.51 mol) and 3-bromopiperidine-2,6-dione (101 mg, 526.01 μmol) in ACN (1.4 mL) was added NaHCO₃ (88.08 mg, 1.05 mmol) and the mixture was stirred at 80° C. for 14 h. LCMS showed 50% peak of the desired mass. The mixture was filtered and the filtrate was concentrated under reduced pressure. The crude product was purified by flash silica gel chromatography (5 g SepaFlash® Silica Flash Column, eluent of 10~20% MeOH in EtOAc, gradient @ 50 mL/min) to afford tert-butyl 4-((3-(4-((2,6-dioxopiperidin-3-yl)amino)phenyl)-3,6-diazabicyclo[3.2.1]octan-6-yl)methyl)piperidine-1-carboxylate (160 mg, 287.69 μmol, 82.31% yield, 92% purity) as a gray solid. MS(M+H)⁺=512.4.

### Step 4. Synthesis of 3-((4-(6-(piperidin-4-ylmethyl)-3,6-diazabicyclo[3.2.1]octan-3-yl)phenyl)amino)piperidine-2,6-dione (5)

To a solution of tert-butyl 4-((3-(4-((2,6-dioxopiperidin-3-yl)amino)phenyl)-3,6-diazabicyclo[3.2.1]octan-6-yl)methyl)piperidine-1-carboxylate (160 mg, 312.71 mol) in DCM (1.5 mL) was added TFA (614.00 mg, 5.39 mmol, 0.4 mL) and the mixture was stirred at 20° C. for 1 h. LCMS showed a main peak with the desired mass. The mixture was concentrated under reduced pressure to afford 3-((4-(6-(piperidin-4-ylmethyl)-3,6-diazabicyclo[3.2.1]octan-3-yl)phenyl) amino)piperidine-2,6-dione (170 mg, crude, TFA) as a blue oil. MS(M+H)⁺=412.2.

### Step 5. Synthesis of N-(2-chlorophenyl)-4-((2-((4-(4-((3-(4-((2,6-dioxopiperidin-3-yl)amino)phenyl)-3,6-diazabicyclo[3.2.1]octan-6-yl)methyl)piperidine-1-carbonyl)phenyl)amino)-5-fluoropyrimidin-4-yl)amino)benzamide (Compound 29)

To a solution of 4-((4-((4-((2-chlorophenyl)carbamoyl)phenyl)amino)-5-fluoropyrimidin-2-yl) amino)benzoic acid (150 mg, 313.89 mol) and HATU (143.22 mg, 376.67 mol) in DMF (2 mL) was added DIPEA (445.20 mg, 3.44 mmol, 0.6 mL) and the mixture was stirred at 20° C. for 15 min. Then a solution of 3-((4-(6-(piperidin-4-ylmethyl)-3,6-diazabicyclo[3.2.1]octan-3-yl)phenyl)amino)piperidine-2,6-dione (170 mg, 323.46 μmol, TFA) in DMF (2 mL) at 0° C. was added and the mixture was stirred at 20° C. for 2 h. LCMS showed the desired mass. The mixture was diluted with water (10 mL) and then filtered. The filter cake was washed with water (5 mL). The filter cake was purified by prep-HPLC (column: Phenomenex luna C18 150*25 mm*10 um; mobile phase: [water (TFA)-ACN]; gradient: 25%-55% B over 10 min) and the eluent was lyophilized. The product was diluted with water (5 mL) and THF (10 mL) and then the mixture was adjusted the pH=7 with saturated NaHCO₃. The mixture was extracted with THF/DCM=1/1 (10 mL×2), the combined organic layer was concentrated under reduced pressure. The product was diluted with EtOAc/MeOH=20/1 (2 mL) and the mixture was stirred at 20° C. for 0.5 h. The mixture was filtered and the filter cake was washed with EtOAc/MeOH=20/1 (10 mL) and water (5 mL). The filter cake was further purified by prep-HPLC (column: Waters Xbridge 150*25 mm*5 um; mobile phase: [water (NH₄HCO₃)-ACN]; gradient: 30%-60% B over min) and lyophilized to afford N-(2-chlorophenyl)-4-((2-((4-(4-((3-(4-((2,6-dioxopiperidin-3-yl)amino)phenyl)-3,6-diazabicyclo[3.2.1]octan-6-yl)methyl)piperidine-1-carbonyl)phenyl)amino)-5-fluoropyrimidin-4-yl)amino)benzamide (18.7 mg, 19.53 μmol, 6.22% yield, 91% purity) as a white solid. MS(M+H)⁺=871.4.

¹H NMR (400 MHz, DMSO-d₆) δ=10.74 (br s, 1H), 9.92 (s, 1H), 9.72 (s, 1H), 9.55 (s, 1H), 8.22 (d, J=3.5 Hz, 1H), 8.03-7.96 (m, 4H), 7.74 (d, J=8.5 Hz, 2H), 7.67-7.63 (m, 1H), 7.57-7.53 (m, 1H), 7.41-7.36 (m, 1H), 7.31-7.26 (m, 3H), 6.68-6.57 (m, 4H), 5.21 (d, J=7.1 Hz, 1H), 4.19-4.11 (m, 1H), 3.60-3.53 (m, 1H), 3.32-3.28 (m, 4H), 3.08-2.99 (m, 2H), 2.94-2.80 (m, 1H), 2.76-2.63 (m, 3H), 2.61-2.53 (m, 2H), 2.48-2.42 (m, 2H), 2.40-2.34 (m, 1H), 2.15-2.05 (m, 1H), 1.88-1.76 (m, 3H), 1.74-1.59 (m, 2H), 1.51-1.45 (m, 1H), 1.13-0.97 (m, 2H).

### Example 30. Synthesis of N-(2-chlorophenyl)-4-((2-((4-(1-((4-(4-((2,6-dioxopiperidin-3-yl)amino)phenyl)piperazin-1-yl)methyl)-6-azaspiro[2.5]octane-6-carbonyl)phenyl)amino)-5-fluoropyrimidin-4-yl)amino)benzamide (Compound 30)

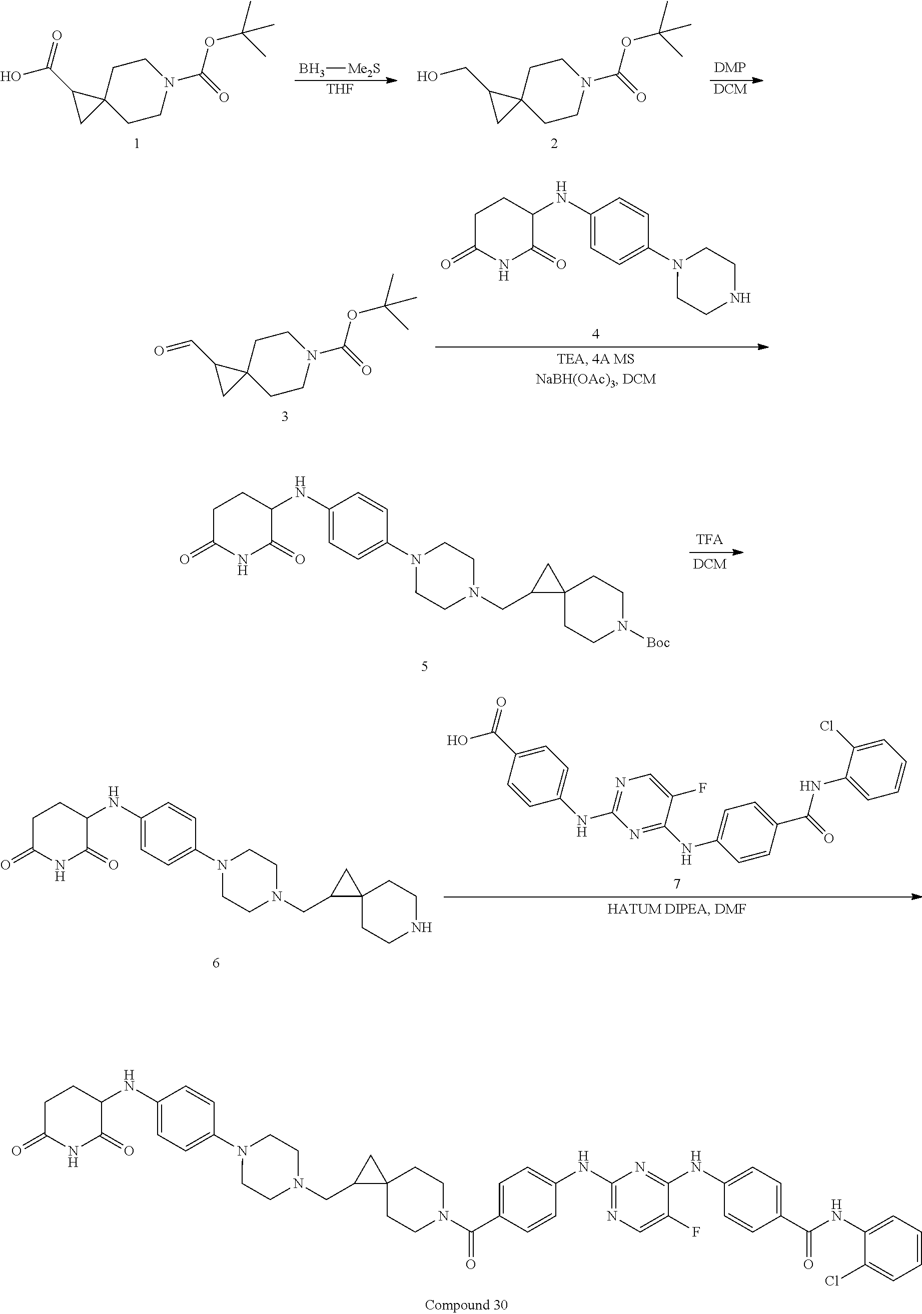

### Step 1. Synthesis of tert-butyl 1-(hydroxymethyl)-6-azaspiro[2.5]octane-6-carboxylate (2)

To a solution of 6-(tert-butoxycarbonyl)-6-azaspiro[2.5]octane-1-carboxylic acid (2 g, 7.83 mmol) in THF (40 mL) was added BH₃-Me₂S (10 M, 1.57 mL) at 0° C. The mixture was stirred at 20° C. for 16 hr. TLC (Petroleum ether:EtOAc=10:1) indicated 6-(tert-butoxycarbonyl)-6-azaspiro[2.5]octane-1-carboxylic acid was consumed completely and one new spot was formed. The reaction mixture was quenched by addition MeOH (20 mL) at 20° C. and stirred for 1 hr. The resulting mixture was concentrated under reduced pressure to afford the residue, which was purified by flash silica gel chromatography (20 g SepaFlash® Silica Flash Column, eluent of 0-40% EtOAc in petroleum ether, gradient @ 40 mL/min) to afford tert-butyl 1-(hydroxymethyl)-6-azaspiro[2.5]octane-6-carboxylate (1.8 g, 7.46 mmol, 95.22% yield) as a colorless oil.

¹H NMR (400 MHz, DMSO-d₆) δ=4.41 (t, J=5.1 Hz, 1H), 3.57-3.42 (m, 2H), 3.41-3.33 (m, 1H), 3.31-3.18 (m, 3H), 1.52-1.43 (m, 1H), 1.39 (s, 9H), 1.36-1.23 (m, 2H), 1.22-1.18 (m, 1H), 0.86-0.77 (m, 1H), 0.48-0.43 (m, 1H).

### Step 2. Synthesis of tert-butyl 1-formyl-6-azaspiro[2.5]octane-6-carboxylate (3)

To a solution of tert-butyl 1-(hydroxymethyl)-6-azaspiro[2.5]octane-6-carboxylate (600 mg, 2.49 mmol) in DCM (25 mL) was added DMP (1.37 g, 3.23 mmol, 1.00 mL), the mixture was stirred at 20° C. for 2 hours. TLC (petroleum ether:EtOAc=1:1) indicated the starting material was consumed completely and one new spot was formed. The reaction mixture was diluted with Na₂SO₃ solution (50 mL) and extracted with EtOAc (20 mL×3). The combined organic layers were washed with NaHCO₃ solution (10 mL×3), bine (10 mL×3), dried over Na₂SO₄, filtered and concentrated in vacuum to afford tert-butyl 1-formyl-6-azaspiro[2.5]octane-6-carboxylate (600 mg) as a colorless oil.

¹H NMR (400 MHz, CDCl₃-d) δ=9.56 (br d, J=3.5 Hz, 1H), 3.58-3.37 (m, 3H), 3.35-3.24 (m, 1H), 1.88-1.79 (m, 1H), 1.78-1.65 (m, 2H), 1.46 (s, 12H), 1.14 (dd, J=4.7, 7.9 Hz, 1H).

### Step 3. Synthesis of tert-butyl 1-((4-(4-((2,6-dioxopiperidin-3-yl)amino)phenyl)piperazin-1-yl)methyl)-6-azaspiro[2.5]octane-6-carboxylate (5)

A mixture of tert-butyl 1-formyl-6-azaspiro[2.5]octane-6-carboxylate (450 mg, 1.88 mmol), 3-((4-(piperazin-1-yl)phenyl)amino)piperidine-2,6-dione (488.61 mg, 1.50 mmol, HCl salt), 4 Å MS (0.2 g, 1.88 mmol) and TEA (761.11 mg, 7.52 mmol, 1.05 mL) in DCM (20 mL) was stirred at 15° C. for 0.5 hours, followed by the addition of NaBH(OAc)₃ (1.59 g, 7.52 mmol). The resulting mixture was stirred at 15° C. for 12 hours. LCMS showed the starting material was consumed completely and 34% peak of desired mass. The mixture was diluted with H₂O (50 mL) and extracted with DCM (20 mL×3). The combined organic layers were dried over Na₂SO₄ and concentrated in vacuum. The residue was purified by flash silica gel chromatography (20 g SepaFlash® Silica Flash Column, eluent of 10~20% EtOAc in petroleum ether, gradient @ 100 mL/min). The residue was further purified by prep-TLC (SiO₂, DCM:MeOH=5:1) to afford tert-butyl 1-((4-(4-((2,6-dioxopiperidin-3-yl)amino)phenyl)piperazin-1-yl)methyl)-6-azaspiro[2.5]octane-6-carboxylate (295 mg, 576.56 μmol, 30.66% yield) as a black solid. MS(M+H)⁺=512.3.

### Step 4. Synthesis of 3-((4-(4-((6-azaspiro[2.5]octan-1-yl)methyl)piperazin-1-yl)phenyl)amino)piperidine-2,6-dione (6)

A mixture of tert-butyl 1-((4-(4-((2,6-dioxopiperidin-3-yl)amino)phenyl)piperazin-1-yl)methyl)-6-azaspiro[2.5]octane-6-carboxylate (160 mg, 312.71 mol) and TFA (2.63 g, 23.08 mmol, 1.71 mL) in DCM (5 mL) was stirred at 15° C. for 1 hour. LCMS showed the starting material was consumed completely and desired mass. The mixture was concentrated in vacuum to afford 3-((4-(4-((6-azaspiro[2.5]octan-1-yl)methyl)piperazin-1-yl)phenyl)amino)piperidine-2, 6-dione (200 mg, 2TFA salt) as a deep blue gum, which was used directly. MS(M+H)⁺=412.2.

### Step 5. Synthesis of N-(2-chlorophenyl)-4-((2-((4-(1-((4-(4-((2,6-dioxopiperidin-3-yl)amino)phenyl)piperazin-1-yl)methyl)-6-azaspiro[2.5]octane-6-carbonyl)phenyl)amino)-5-fluoropyrimidin-4-yl)amino)benzamide (Compound 30)

A mixture of 4-((4-((4-((2-chlorophenyl)carbamoyl)phenyl)amino)-5-fluoropyrimidin-2-yl)amino)benzoic acid (150 mg, 313.89 mol), HATU (155.16 mg, 408.06 μmol) and DIPEA (405.68 mg, 3.14 mmol, 546.74 μL) in DMF (2 mL) was stirred at 15° C. for 15 minutes, then 3-((4-(4-((6-azaspiro[2.5]octan-1-yl)methyl)piperazin-1-yl)phenyl)amino)piperidine-2, 6-dione (200 mg, 312.70 μmol, 2TFA salt) was added. The resulting mixture was stirred at 15° C. for 1 hour. LCMS showed 3-((4-(4-((6-azaspiro[2.5]octan-1-yl)methyl)piperazin-1-yl)phenyl)amino)piperidine-2, 6-dione was consumed completely and 43% peak of desired mass. The mixture was treated with TFA to adjust pH<7 and filtered. The filtrate was purified by prep-HPLC (column: Phenomenex luna C18 150*40 mm*15 um; mobile phase: [water(TFA)-ACN]; gradient: 15%-45% B over 10 min). The impure product was added to a mixture (EtOAc:THF:ACN=4:1:1, 80 mL) and treated with NaHCO₃ solution (10 mL). The organic phase was separated and concentrated in vacuum. The residue was further purified by prep-TLC (SiO₂, DCM:MeOH=8:1) to afford N-(2-chlorophenyl)-4-((2-((4-(1-((4-(4-((2,6-dioxopiperidin-3-yl)amino)phenyl)piperazin-1-yl)methyl)-6-azaspiro[2.5]octane-6-carbonyl)phenyl)amino)-5-fluoropyrimidin-4-yl)amino)benzamide (48.4 mg, 53.10 μmol, 16.92% yield, 95.6% purity) as a gray solid. MS(M+H)⁺=871.3.

¹H NMR (400 MHz, DMSO-d₆) δ=10.76 (s, 1H), 9.93 (s, 1H), 9.73 (s, 1H), 9.56 (s, 1H), 8.23 (d, J=3.5 Hz, 1H), 8.08-7.94 (m, 4H), 7.74 (d, J=8.7 Hz, 2H), 7.69-7.61 (m, 1H), 7.56 (dd, J=1.2, 7.9 Hz, 1H), 7.44-7.36 (m, 1H), 7.36-7.23 (m, 3H), 6.74 (br d, J=8.8 Hz, 2H), 6.60 (d, J=8.8 Hz, 2H), 5.37 (d, J=7.2 Hz, 1H), 4.24-4.13 (m, 1H), 3.86-3.69 (m, 1H), 2.95-2.92 (m, 4H), 2.78-2.67 (m, 2H), 2.62-2.53 (m, 6H), 2.46-2.42 (m, 2H), 2.32-2.21 (m, 1H), 2.15-2.06 (m, 1H), 1.89-1.78 (m, 1H), 1.57-1.54 (m, 1H), 1.51-1.43 (m, 1H), 1.43-1.35 (m, 1H), 1.29-1.18 (m, 1H), 0.85-0.74 (m, 1H), 0.55-0.50 (m, 1H), 0.16-0.13 (m, 1H).

### Example 31. Synthesis of N-(2-chlorophenyl)-4-((2-((4-(6-((1-(4-((2,6-dioxopiperidin-3-yl)amino)phenyl)piperidin-4-yl)methyl)-2,6-diazaspiro[3.3]heptane-2-carbonyl)phenyl)amino)-5-fluoropyrimidin-4-yl)amino)benzamide (Compound 31)

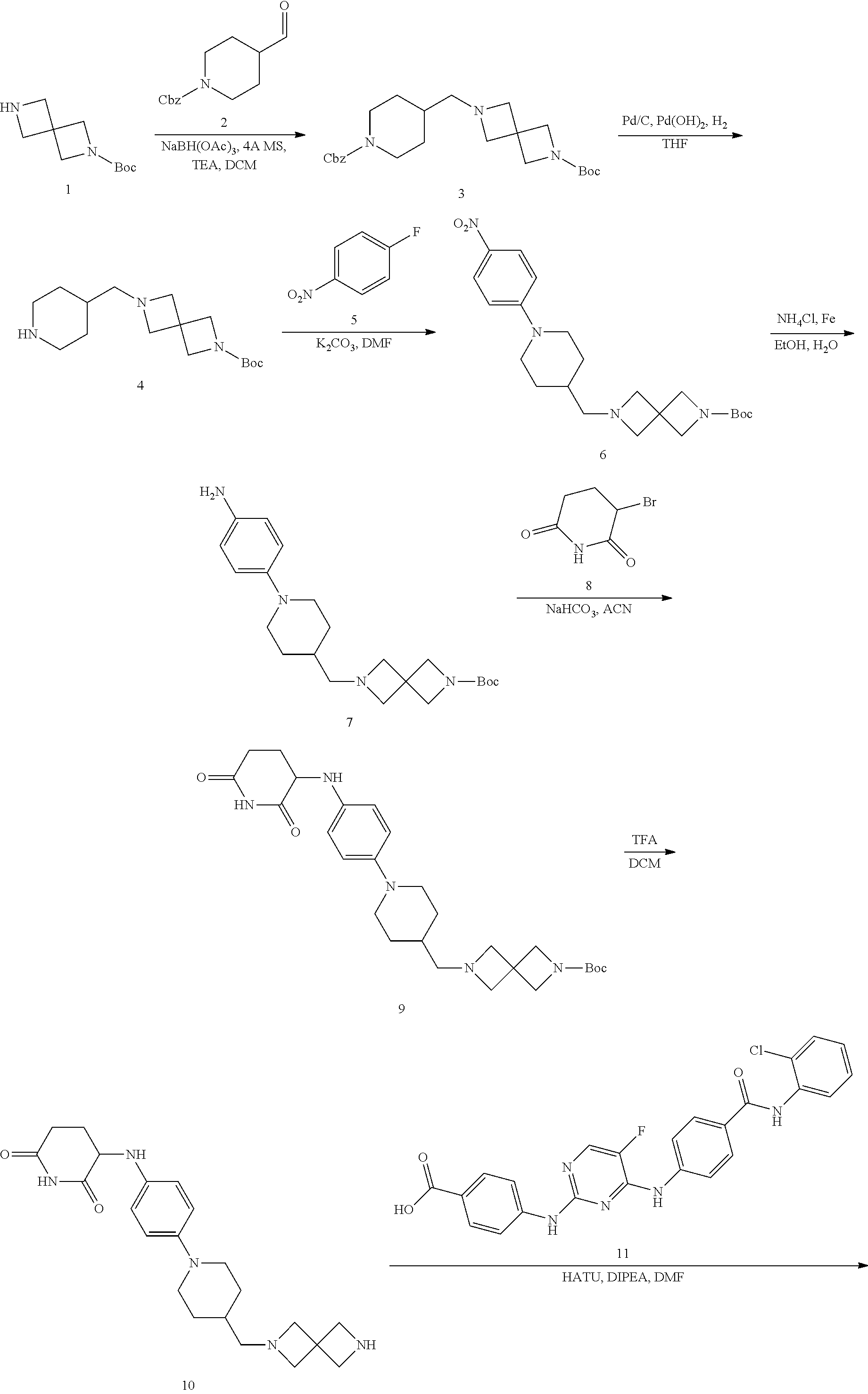

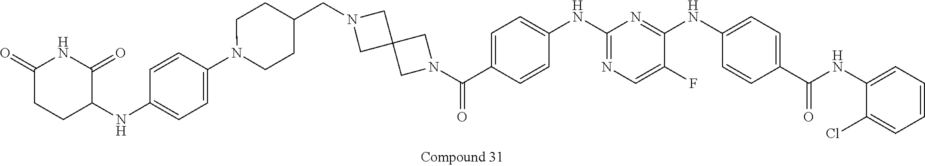 [SMILES: O=C1CCC(Nc2ccc(N3CCC(CN4CC5(C4)CN(C(=O)c4ccc(Nc6ncc(F)c(Nc7ccc(C(=O)Nc8ccccc8Cl)cc7)n6)cc4)C5)CC3)cc2)C(=O)N1]

### Step 1. Synthesis of tert-butyl 6-((1-((benzyloxy)carbonyl)piperidin-4-yl)methyl)-2,6-diazaspiro[3.3]heptane-2-carboxylate (3)

To a solution of tert-butyl 2,6-diazaspiro[3.3]heptane-2-carboxylate (3.37 g, 11.69 mmol), TEA (3.55 g, 35.07 mmol, 4.88 mL), 4 Å MS (500 mg) and benzyl 4-formylpiperidine-1-carboxylate (2.89 g, 11.69 mmol) in DCM (33 mL) was stirred at 25° C. for 1 hr. And the reaction mixture was added NaBH(OAc)₃ (7.43 g, 35.07 mmol) and stirred at 25° C. for 14 hr. LCMS showed tert-butyl 2,6-diazaspiro[3.3]heptane-2-carboxylate was consumed completely and desired mass was detected. The reaction mixture was diluted with H₂O (30 mL) at 20° C. and then extracted with EtOAc (50 mL×3). The combined organic layers were dried over Na₂ SO₄, filtered and concentrated under reduced pressure to give a residue. The residue was purified by flash silica gel chromatography (ISCO®; 40 g SepaFlash® Silica Flash Column, eluent of 90% EtOAc in petroleum ether, gradient @ 100 mL/min) to afford tert-butyl 6-[(1-benzyloxycarbonyl-4-piperidyl)methyl]-2,6-diazaspiro[3.3]heptane-2-carboxylate (4.35 g, 8.71 mmol, 74.50% yield, 86% purity) as a colorless oil. MS(M+H)⁺=430.2.

### Step 2. Synthesis of tert-butyl 6-(piperidin-4-ylmethyl)-2,6-diazaspiro[3.3]heptane-2-carboxylate (4)

To a solution of tert-butyl 6-((1-((benzyloxy)carbonyl)piperidin-4-yl)methyl)-2,6-diazaspiro[3.3]heptane-2-carboxylate (2.91 g, 5.83 mmol) in THF (30 mL) was added Pd/C (1 g, 10% purity) and Pd(OH)₂/C (1 g, 20% purity) under N₂ atmosphere. The suspension was degassed under vacuum and purged with H₂ several times. The mixture was stirred under H₂ (15 Psi) atmosphere at 25° C. for 20 hours. LCMS showed tert-butyl 6-((1-((benzyloxy)carbonyl)piperidin-4-yl)methyl)-2,6-diazaspiro[3.3]heptane-2-carboxylate was consumed completely. The mixture was filtered and washed with THF (200 mL). The filtrate was concentrated under reduced pressure to afford tert-butyl 6-(piperidin-4-ylmethyl)-2,6-diazaspiro[3.3]heptane-2-carboxylate (1.85 g, crude) as a white solid. MS(M+H)⁺=296.1.

### Step 3. Synthesis of tert-butyl 6-((1-(4-nitrophenyl)piperidin-4-yl)methyl)-2,6-diazaspiro[3.3]heptane-2-carboxylate (6)

To a solution of tert-butyl 6-(piperidin-4-ylmethyl)-2,6-diazaspiro[3.3]heptane-2-carboxylate (950 mg, 3.22 mmol) and 1-fluoro-4-nitrobenzene (907.49 mg, 6.43 mmol) in DMF (9.5 mL) was added K₂CO₃ (2.22 g, 16.08 mmol). The mixture was stirred at 25° C. for 16 hr. LCMS showed tert-butyl 6-(piperidin-4-ylmethyl)-2,6-diazaspiro[3.3]heptane-2-carboxylate was consumed completely. The reaction mixture was diluted with H₂O (50 mL) at 20° C., and then extracted with EtOAc (20 mL×3). The combined organic layers were washed with brine (20 mL), dried over Na₂SO₄, filtered and concentrated under reduced pressure to give a residue. The residue was purified by flash silica gel chromatography (ISCO®; 40 g SepaFlash® Silica Flash Column, eluent of 60% EtOAc in petroleum ether, gradient @ 100 mL/min) to afford tert-butyl 6-((1-(4-nitrophenyl)piperidin-4-yl)methyl)-2,6-diazaspiro[3.3]heptane-2-carboxylate (915 mg, 2.04 mmol, 63.53% yield, 93% purity) as a yellow solid. MS(M+H)⁺=417.2.

### Step 4. Synthesis of tert-butyl 6-((1-(4-aminophenyl)piperidin-4-yl)methyl)-2,6-diazaspiro[3.3]heptane-2-carboxylate (7)

To a solution of tert-butyl 6-((1-(4-nitrophenyl)piperidin-4-yl)methyl)-2,6-diazaspiro[3.3]heptane-2-carboxylate (915 mg, 2.04 mmol) in EtOH (30 mL) and H₂O (6 mL) were added NH₄Cl (1.09 g, 20.43 mmol) and Fe (570.47 mg, 10.22 mmol). The resulting mixture was stirred at 80° C. for 1 hr. LCMS showed the starting material was consumed completely. The reaction mixture was diluted with saturated NaHCO₃ solution (40 mL) and extracted with EtOAc (20 mL×3). The combined organic layers were dried over Na₂SO₄, filtered and concentrated under reduced pressure to afford tert-butyl 6-[[1-(4-aminophenyl)-4-piperidyl]methyl]-2, 6-diazaspiro[3.3]heptane-2-carboxylate (820 mg, crude) as a brown solid. MS(M+H)⁺=387.2.

### Step 5. Synthesis of tert-butyl 6-((1-(4-((2,6-dioxopiperidin-3-yl)amino)phenyl)piperidin-4-yl)methyl)-2,6-diazaspiro[3.3]heptane-2-carboxylate (9)

To a solution of tert-butyl 6-((1-(4-aminophenyl)piperidin-4-yl)methyl)-2,6-diazaspiro[3.3]heptane-2-carboxylate (160 mg, 413.94 mol) and 3-bromopiperidine-2,6-dione (198.70 mg, 1.03 mmol) in ACN (0.8 mL) was added NaHCO₃ (201.69 mg, 2.40 mmol, 93.42 μL). The mixture was stirred at 80° C. for 5 hr. LCMS showed the starting material was consumed completely. The reaction mixture was diluted with H₂O (20 mL) at 20° C., and then extracted with EtOAc (10 mL×2). The combined organic layers were dried over Na₂SO₄, filtered and concentrated under reduced pressure to give a residue. The residue was purified by flash silica gel chromatography (ISCO®; 20 g SepaFlash® Silica Flash Column, eluent of 0~20% MeOH/EtOAc @ 50 mL/min) to afford tert-butyl 6-((1-(4-((2,6-dioxopiperidin-3-yl)amino)phenyl)piperidin-4-yl)methyl)-2,6-diazaspiro[3.3]heptane-2-carboxylate (130 mg, 261.24 μmol, 63.11% yield) as a black solid. MS(M+H)⁺=498.4.

### Step 6. Synthesis of 3-((4-(4-((2,6-diazaspiro[3.3]heptan-2-yl)methyl)piperidin-1-yl)phenyl)amino)piperidine-2,6-dione (10)

To a solution of tert-butyl 6-((1-(4-((2,6-dioxopiperidin-3-yl)amino)phenyl)piperidin-4-yl)methyl)-2,6-diazaspiro[3.3]heptane-2-carboxylate (130 mg, 261.24 μmol) in DCM (1.5 mL) was added TFA (767.50 mg, 6.73 mmol, 0.5 mL). The mixture was stirred at 25° C. for 0.5 hr. LCMS showed the starting material was consumed completely. The mixture was concentrated under reduced pressure to afford 3-((4-(4-((2,6-diazaspiro[3.3]heptan-2-yl)methyl)piperidin-1-yl)phenyl)amino)piperidine-2,6-dione (133 mg, 260.00 μmol, 99.53% yield, TFA) as a green oil. MS(M+H)⁺=398.3.

### Step 7. Synthesis of N-(2-chlorophenyl)-4-((2-((4-(6-((1-(4-((2,6-dioxopiperidin-3-yl)amino)phenyl)piperidin-4-yl)methyl)-2,6-diazaspiro[3.3]heptane-2-carbonyl)phenyl)amino)-5-fluoropyrimidin-4-yl)amino)benzamide (Compound 31)

To a solution of 4-((4-((4-((2-chlorophenyl)carbamoyl)phenyl)amino)-5-fluoropyrimidin-2-yl)amino)benzoic acid (115 mg, 240.65 mol) in DMF (1.3 mL) were added HATU (114.38 mg, 300.81 μmol) and DIPEA (259.19 mg, 2.01 mmol, 349.31 μL). After stirring at 25° C. for 0.5 hr, 3-((4-(4-((2, 6-diazaspiro[3.3]heptan-2-yl)methyl)piperidin-1-yl)phenyl)amino)piperidine-2, 6-dione (129.85 mg, 200.54 μmol, TFA) was added and the resulting mixture was stirred at 25° C. for 2 hr. LCMS showed 3-((4-(4-((2,6-diazaspiro[3.3]heptan-2-yl)methyl)piperidin-1-yl)phenyl) amino)piperidine-2,6-dione was consumed completely. The reaction mixture was diluted with H₂O (20 mL) and then extracted with EtOAc (10 mL×2). The combined organic layers were washed with brine (10 mL), dried over Na₂SO₄, filtered and concentrated under reduced pressure to give a residue. The residue was purified by prep-HPLC (column: Waters Xbridge 150*25 mm*5 um; mobile phase: [water (NH₄HCO₃)-ACN]; gradient: 32%-62% B over min) and lyophilized to afford N-(2-chlorophenyl)-4-((2-((4-(6-((1-(4-((2,6-dioxopiperidin-3-yl)amino)phenyl)piperidin-4-yl)methyl)-2,6-diazaspiro[3.3]heptane-2-carbonyl)phenyl)amino)-5-fluoropyrimidin-4-yl)amino)benzamide (41 mg, 43.99 μmol, 21.94% yield, 92% purity) as a white solid. MS(M+H)⁺=857.3.

¹H NMR (400 MHz, DMSO-d₆) δ=10.85-10.66 (m, 1H), 9.96 (br s, 1H), 9.84-9.71 (m, 1H), 9.65 (s, 1H), 8.24 (d, J=3.5 Hz, 1H), 8.10-7.98 (m, 4H), 7.76 (d, J=8.7 Hz, 2H), 7.65 (d, J=8.2 Hz, 1H), 7.61-7.51 (m, 3H), 7.44-7.35 (m, 1H), 7.33-7.25 (m, 1H), 6.73 (d, J=8.7 Hz, 2H), 6.59 (d, J=8.9 Hz, 2H), 5.36 (d, J=7.3 Hz, 1H), 4.44-4.33 (m, 2H), 4.24-4.14 (m, 1H), 4.13-4.00 (m, 2H), 3.27-3.13 (m, 5H), 2.78-2.69 (m, 1H), 2.60-2.55 (m, 1H), 2.50-2.40 (m, 2H), 2.20-2.15 (m, 2H), 2.13-2.04 (m, 1H), 1.89-1.77 (m, 1H), 1.70-1.60 (m, 2H), 1.32-1.11 (m, 4H).

### Example 32. Synthesis of N-(2-chlorophenyl)-4-((2-((4-(7-((1-(4-((2,6-dioxopiperidin-3-yl)amino)phenyl)piperidin-4-yl)methyl)-2,7-diazaspiro[4.4]nonane-2-carbonyl)phenyl)amino)-5-fluoropyrimidin-4-yl)amino)benzamide (Compound 32)

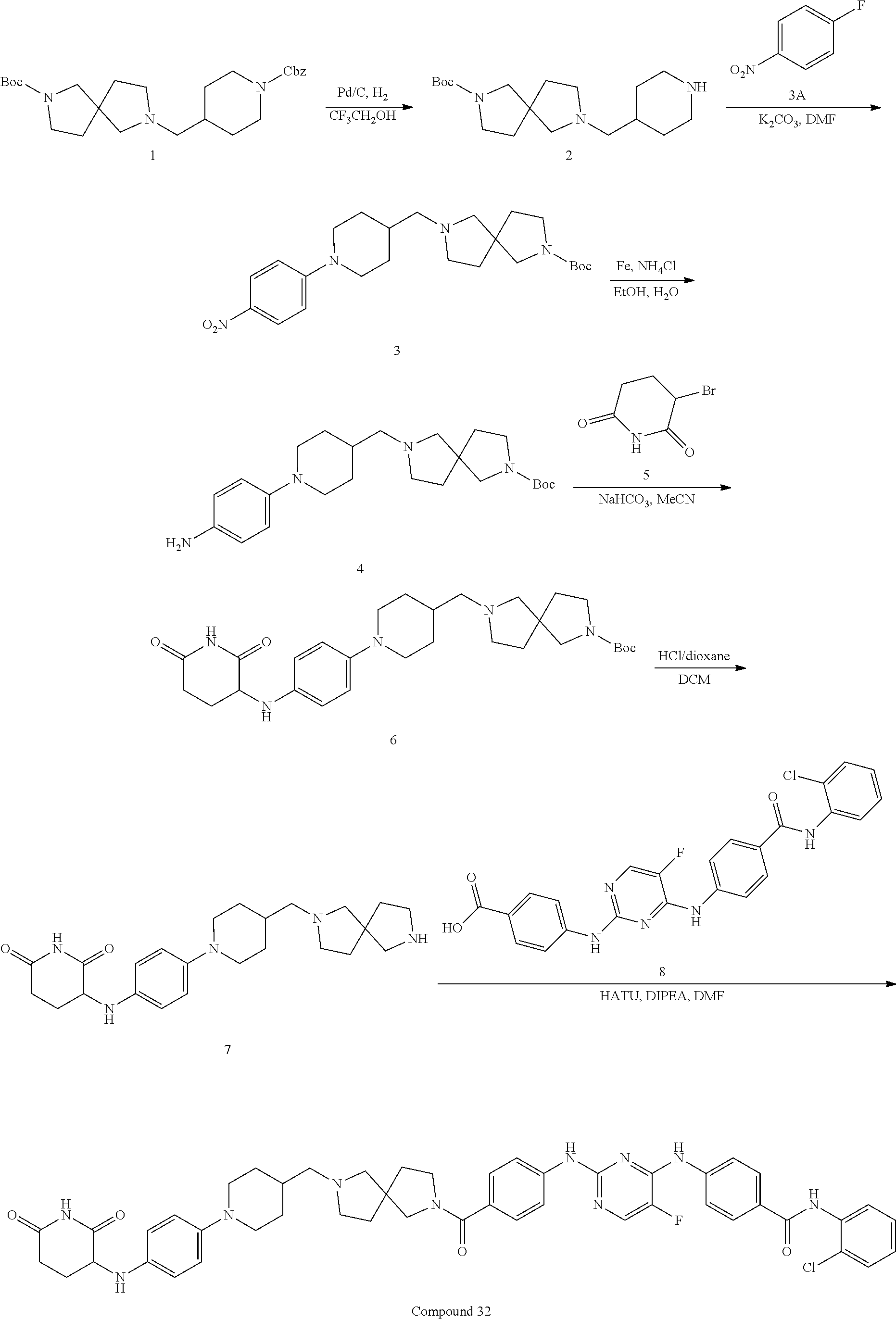

### Step 1. Synthesis of tert-butyl 7-(piperidin-4-ylmethyl)-2,7-diazaspiro[4.4]nonane-2-carboxylate (2)

To a solution of tert-butyl 7-((1-((benzyloxy)carbonyl)piperidin-4-yl)methyl)-2,7-diazaspiro[4.4]nonane-2-carboxylate (1 g, 2.19 mmol) in CF₃CH₂OH (10 mL) was added Pd/C (232.56 mg, 218.53 mol, 10% purity) under N₂ atmosphere. The resulting mixture was stirred at 25° C. under H₂ atmosphere(15 Psi) for 12 hr. LCMS showed the starting material was consumed completely and 56% of desired compound. The reaction mixture was filtered and the filtrate was concentrated under reduced pressure to afford tert-butyl 7-(piperidin-4-ylmethyl)-2,7-diazaspiro[4.4]nonane-2-carboxylate (1.1 g, crude) as a brown solid. MS(M+H)⁺=324.2.

### Step 2. Synthesis of tert-butyl 7-((1-(4-nitrophenyl)piperidin-4-yl)methyl)-2,7-diazaspiro[4.4]nonane-2-carboxylate (3)

To a solution of 1-fluoro-4-nitrobenzene (436.20 mg, 3.09 mmol, 327.97 μL) and tert-butyl 7-(piperidin-4-ylmethyl)-2,7-diazaspiro[4.4]nonane-2-carboxylate (1 g, 3.09 mmol) in DMF (10 mL) was added K₂CO₃ (1.28 g, 9.27 mmol). The mixture was stirred at 50° C. for 12 hr. LCMS showed 54% peak of desired compound. The reaction mixture was diluted with H₂O (50 mL) and extracted with solvent EtOAc (20 mL×3). The combined organic layers were washed with brine (20 mL), dried over Na₂SO₄, filtered and concentrated under reduced pressure to give a residue. The residue was purified by flash silica gel chromatography (ISCO®; 20 g SepaFlash® Silica Flash Column, Eluent of 50% EtOAc in petroleum ether, gradient @ 75 mL/min) to afford tert-butyl 7-[[1-(4-nitrophenyl)-4-piperidyl]methyl]-2,7-diazaspiro[4.4]nonane-2-carboxylate (617 mg, 1.39 mmol, 44.89% yield, 100% purity) as a yellow solid. MS(M+H)⁺=445.3.

### Step 3. Synthesis of tert-butyl 7-((1-(4-aminophenyl)piperidin-4-yl)methyl)-2,7-diazaspiro[4.4]nonane-2-carboxylate (4)

To a solution of tert-butyl 7-((1-(4-nitrophenyl)piperidin-4-yl)methyl)-2,7-diazaspiro[4.4]nonane-2-carboxylate (650 mg, 1.46 mmol) in EtOH (10 mL) and H₂O (4 mL) were added Fe (408.26 mg, 7.31 mmol) and NH₄Cl (391.05 mg, 7.31 mmol). The resulting mixture was stirred at 80° C. for 2 hr. LCMS showed the starting material was consumed completely and 86% peak of desired mass. The mixture was diluted with NaHCO₃ solution (100 mL) and extracted with EtOAc (50 mL×3). The combined organic layers were dried over Na₂ SO₄, filtered and concentrated under reduced pressure to give a residue. The residue was purified by prep-HPLC (column: Phenomenex luna C18 150*40 mm*15 um; mobile phase: [water (FA)-ACN]; gradient: 5%-35% B over 10 min) to afford tert-butyl 7-((1-(4-aminophenyl)piperidin-4-yl)methyl)-2,7-diazaspiro[4.4]nonane-2-carboxylate (317 mg, 711.10 μmol, 48.64% yield, 93% purity) and tert-butyl 7-((1-(4-aminophenyl)piperidin-4-yl)methyl)-2,7-diazaspiro[4.4]nonane-2-carboxylate (163 mg, 314.53 μmol, 21.51% yield, 80% purity) as a white solid. MS(M+H)⁺=415.4.

### Step 4. Synthesis of tert-butyl 7-((1-(4-((2,6-dioxopiperidin-3-yl)amino)phenyl)piperidin-4-yl) methyl)-2,7-diazaspiro[4.4]nonane-2-carboxylate (6)

To a solution of 3-bromopiperidine-2,6-dione (206.33 mg, 1.07 mmol) and tert-butyl 7-((1-(4-aminophenyl)piperidin-4-yl)methyl)-2,7-diazaspiro[4.4]nonane-2-carboxylate (297 mg, 716.38 μmol) in MeCN (0.2 mL) was added NaHCO₃ (180.54 mg, 2.15 mmol). The mixture was stirred at 80° C. for 4 h. LCMS showed 30% peak of desired compound was detected. The reaction mixture was filtered and the filtrate was concentrated. The residue was purified by prep-HPLC (column: Phenomenex luna C18 150*25 mm*10 um; mobile phase: [water (FA)-ACN]; gradient: 1%-30% B over 10 min) and lyophilized to afford tert-butyl 7-((1-(4-((2,6-dioxopiperidin-3-yl)amino)phenyl)piperidin-4-yl)methyl)-2,7-diazaspiro[1.4]nonane-2-carboxylate (257 mg, 488.89 μmol, 68.24% yield, 100% purity) as a white solid. MS(M+H)⁺=526.3.

### Step 5. Synthesis of 3-((4-(4-((2,7-diazaspiro[4.4]nonan-2-yl)methyl)piperidin-1-yl)phenyl)amino)piperidine-2,6-dione (7)

To a solution of tert-butyl 7-((1-(4-((2,6-dioxopiperidin-3-yl)amino)phenyl)piperidin-4-yl)methyl)-2,7-diazaspiro[4.4]nonane-2-carboxylate (257 mg, 488.89 μmol) in DCM (5 mL) was added HCl/dioxane (4 M, 733.33 μL). The mixture was stirred at 25° C. for 3.5 hr. LCMS showed 9% of the starting material remained and 53% peak of desired compound. The reaction mixture was concentrated under reduced pressure to afford 3-((4-(4-((2,7-diazaspiro[4.4]nonan-2-yl)methyl)piperidin-1-yl)phenyl)amino)piperidine-2,6-dione (245 mg, crude, HCl) as a white solid. MS(M+H)⁺=426.3.

### Step 6. Synthesis of N-(2-chlorophenyl)-4-((2-((4-(7-((1-(4-((2,6-dioxopiperidin-3-yl)amino)phenyl)piperidin-4-yl)methyl)-2,7-diazaspiro[4.4]nonane-2-carbonyl)phenyl)amino)-5-fluoropyrimidin-4-yl)amino)benzamide (Compound 32)

To a solution of 3-((4-(4-((2,7-diazaspiro[4.4]nonan-2-yl)methyl)piperidin-1-yl)phenyl)amino)piperidine-2,6-dione (100 mg, 216.44 μmol, HCl) and 4-((4-((4-((2-chlorophenyl)carbamoyl)phenyl)amino)-5-fluoropyrimidin-2-yl)amino)benzoic acid (103.43 mg, 216.44 μmol) in DMF (1 mL) were added DIPEA (83.92 mg, 649.31 μmol, 113.10 μL) and HATU (98.76 mg, 259.73 μmol). The mixture was stirred at 25° C. for 2 hr. LCMS showed 51% peak of desired compound was detected. The residue was diluted with H₂O (20 mL) and extracted with solvent EtOAc (10 mL×3). The combined organic layers were washed with brine (10 mL), dried over Na₂SO₄, filtered and concentrated under reduced pressure to give a residue. The residue was purified by prep-HPLC (column: Waters Xbridge C18 150*50 mm*10 um; mobile phase: [water (NH₄HCO₃)-ACN]; gradient: 33%-63% B over min) and lyophilized to afford N-(2-chlorophenyl)-4-((2-((4-(7-((1-(4-((2,6-dioxopiperidin-3-yl)amino)phenyl)piperidin-4-yl)methyl)-2,7-diazaspiro[4.4]nonane-2-carbonyl)phenyl)amino)-5-fluoropyrimidin-4-yl)amino)benzamide (45.3 mg, 48.96 μmol, 22.62% yield, 95.7% purity) as a white solid. MS(M+H)⁺=885.4.

¹H NMR (400 MHz, DMSO-d₆) δ=10.75 (s, 1H), 9.93 (s, 1H), 9.73 (s, 1H), 9.62-9.55 (m, 1H), 8.23 (d, J=3.2 Hz, 1H), 8.10-7.98 (m, 4H), 7.78-7.72 (m, 2H), 7.65 (br d, J=8.2 Hz, 1H), 7.56 (d, J=7.9 Hz, 1H), 7.50-7.37 (m, 3H), 7.32-7.26 (m, 1H), 6.78-6.67 (m, 2H), 6.62-6.54 (m, 2H), 5.42-5.29 (m, 1H), 4.25-4.11 (m, 1H), 3.57-3.41 (m, 4H), 2.82-2.57 (m, 3H), 2.46-2.25 (m, 6H), 2.24-2.06 (m, 3H), 1.91-1.59 (m, 8H), 1.52-1.36 (m, 1H), 1.26-1.08 (m, 2H).

### Example 33. Synthesis of N-(2-chlorophenyl)-4-[[2-[4-[8-[[1-[4-[(2,6-dioxo-3-piperidyl)amino]phenyl]-4-piperidyl]methyl]-2,8-diazaspiro[4.5]decane-2-carbonyl]anilino]-5-fluoro-pyrimidin-4-yl]amino]benzamide (Compound 33)

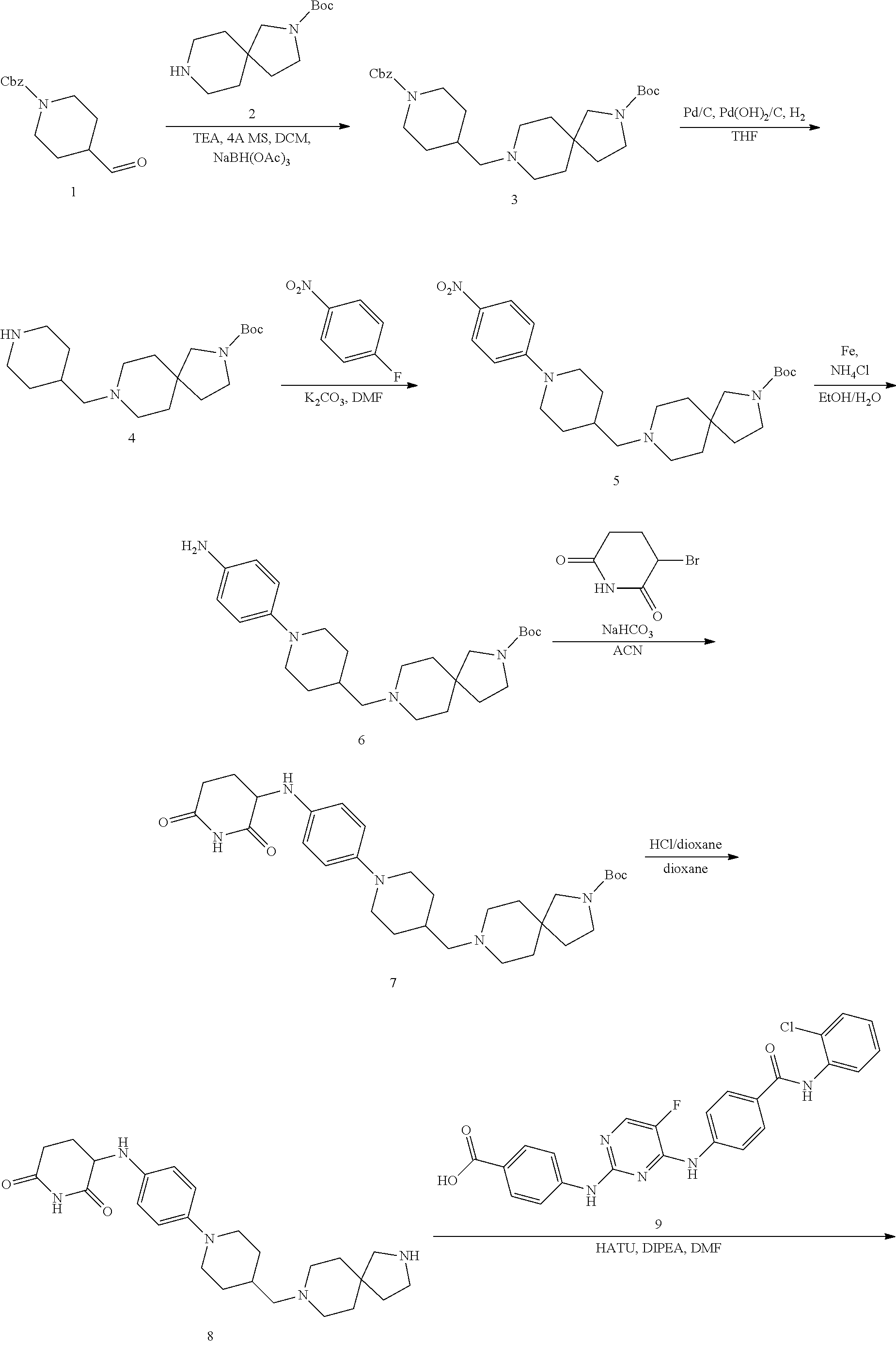

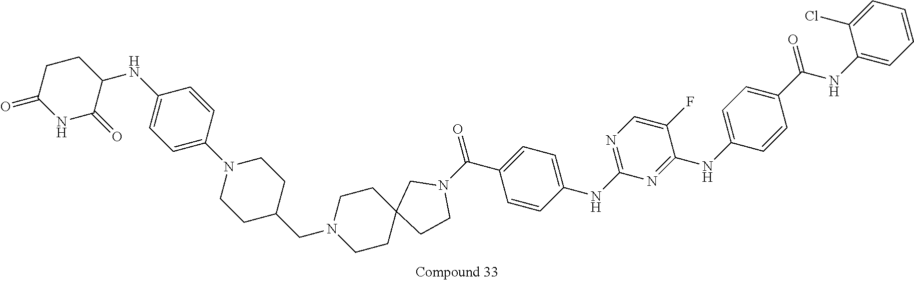 [SMILES: O=C1CCC(Nc2ccc(N3CCC(CN4CCC5(CC4)CCN(C(=O)c4ccc(Nc6ncc(F)c(Nc7ccc(C(=O)Nc8ccccc8Cl)cc7)n6)cc4)C5)CC3)cc2)C(=O)N1]

### Step 1. Synthesis of tert-butyl 8-[(1-benzyloxycarbonyl-4-piperidyl)methyl]-2,8-diazaspiro[4.5] decane-2-carboxylate (3)

To a solution of benzyl 4-formlypiperidine-1-carboxylate (400 mg, 1.62 mmol) and tert-butyl 2,8-diazaspiro[4.5]decane-2-carboxylate (388.76 mg, 1.62 mmol) in DCM (8 mL) was added TEA (491.03 mg, 4.85 mmol, 675.42 μL) and 4 Å MS (100 mg). After stirring at 15° C. for 30 min, NaBH(OAc)₃ (1.03 g, 4.85 mmol) was added and the resulting mixture was stirred at 15° C. for 5 hr. LCMS showed a main peak (94%) with the desired mass. The reaction mixture was diluted with aqueous NH₄Cl solution (20 mL) and the layers were separated. The aqueous phase was extracted with EtOAc (20 mL×3). The combined organic phase was dried over Na₂SO₄, filtered and concentrated under reduced pressure. The residue was purified by flash silica gel chromatography (12 g SepaFlash® Silica Flash Column, eluent of 80% EtOAc in petroleum ether, gradient @ 60 mL/min) to afford tert-butyl 8-[(1-benzyloxycarbonyl-4-piperidyl)methyl]-2,8-diazaspiro[4.5]decane-2-carboxylate (850 mg, 1.37 mmol, 84.68% yield, 95% purity) as colorless oil. MS(M+H)⁺=472.3.

¹H NMR (400 MHz, CDCl₃-d) δ=7.33-7.21 (m, 5H), 5.05 (s, 2H), 4.22-4.01 (m, 2H), 3.37-3.22 (m, 2H), 3.19-2.97 (m, 2H), 2.75-2.60 (m, 2H), 2.54-2.00 (m, 9H), 1.77-1.57 (m, 6H), 1.39 (s, 9H), 1.13-0.94 (m, 2H).

### Step 2. Synthesis of tert-butyl 8-(4-piperidylmethyl)-2,8-diazaspiro[4.5]decane-2-carboxylate (4)

To a mixture of Pd/C (191.80 mg, 180.23 μmol, 10% purity) and Pd(OH)₂/C (126.55 mg, 180.23 μmol, 20% purity) in THF (5 mL) was added tert-butyl 8-[(1-benzyloxycarbonyl-4-piperidyl)methyl]-2,8-diazaspiro[4.5]decane-2-carboxylate (850 mg, 1.80 mmol) in THF (5 mL) under N₂ atmosphere. The suspension was degassed and purged with H₂ for 3 times. The mixture was stirred under H₂ (50 Psi) atmosphere at 30° C. for 16 hr. LCMS showed a peak (38%) with the mass of desired product. The reaction mixture was filtered through celite and concentrated under reduced pressure to afford tert-butyl 8-(4-piperidylmethyl)-2,8-diazaspiro[4.5]decane-2-carboxylate (600 mg, crude) as gray oil, which was used for the next step directly. MS(M+H)⁺=338.2.

### Step 3. Synthesis of tert-butyl 8-[[1-(4-nitrophenyl)-4-piperidyl]methyl]-2,8-diazaspiro[4.5]decane-2-carboxylate (5)

To a solution of tert-butyl 8-(4-piperidylmethyl)-2,8-diazaspiro[4.5]decane-2-carboxylate (600 mg, 1.78 mmol) in DMF (10 mL) was added K₂CO₃ (737.10 mg, 5.33 mmol) and 1-fluoro-4-nitro-benzene (250.84 mg, 1.78 mmol, 188.61 μL). The mixture was stirred at 80° C. for 16 hr. LCMS showed a peak (65%) with the mass of desired product. The reaction mixture was diluted with water (50 mL) and extracted with EtOAc (20 mL×3). The combined organic layers were washed with brine (10 mL×3), dried over Na₂ SO₄, filtered and concentrated. The residue was purified by flash silica gel chromatography (12 g SepaFlash® Silica Flash Column, eluent of 0~20% EtOAc/petroleum ether, gradient @ 60 mL/min) to afford the crude product, which was further purified by trituration with 5 ml of EtOAc and 3 ml of MeCN to afford tert-butyl 8-[[1-(4-nitrophenyl)-4-piperidyl]methyl]-2,8-diazaspiro[4.5]decane-2-carboxylate (250 mg, 501.54 μmol, 28.21% yield, 92% purity) as yellow solid. MS(M+H)⁺=459.3.

### Step 4. Synthesis of tert-butyl 8-[[1-(4-aminophenyl)-4-piperidyl]methyl]-2,8-diazaspiro[4.5]decane-2-carboxylate (6)

To a solution of tert-butyl 8-[[1-(4-nitrophenyl)-4-piperidyl]methyl]-2,8-diazaspiro[4.5]decane-2-carboxylate (250 mg, 545.15 μmol) in EtOH (3 mL) and H₂O (3 mL) were added Fe (91.33 mg, 1.64 mmol) and NH₄Cl (145.80 mg, 2.73 mmol). The mixture was stirred at 80° C. for 1 hr. LCMS showed a main peak with desired mass. The reaction mixture was filtered through a celite pad and concentrated under reduced pressure. The residue was diluted with water (20 mg) and extracted with EtOAc (20 ml×3). To the aqueous phase was added solid NaHCO₃ (1 g) and the mixture was extracted with DCM (20 ml×3). The combined organic phase was dried over Na₂SO₄ and concentrated under reduced pressure to afford tert-butyl 8-[[1-(4-aminophenyl)-4-piperidyl]methyl]-2,8-diazaspiro[4.5]decane-2-carboxylate (200 mg, 457.29 μmol, 83.88% yield, 98% purity) as brown solid. MS(M+H)⁺=429.2.

### Step 5. Synthesis of tert-butyl 8-[[1-[4-[(2,6-dioxo-3-piperidyl)amino]phenyl]-4-piperidyl]methyl]-2,8-diazaspiro[4.5]decane-2-carboxylate (7)

To a solution of tert-butyl 8-[[1-(4-aminophenyl)-4-piperidyl]methyl]-2,8-diazaspiro[4.5]decane-2-carboxylate (180 mg, 419.96 μmol) and 3-bromopiperidine-2,6-dione (201.59 mg, 1.05 mmol) in ACN (1 mL) was added NaHCO₃ (141.12 mg, 1.68 mmol). The mixture was stirred at 80° C. for 16 hr. LCMS showed a peak (76%, 254 nm) with the mass of desired product. The reaction mixture was diluted with EtOAc (10 mL) and filtered to afford a residue. The residue was washed with water to afford crude tert-butyl 8-[[1-[4-[(2,6-dioxo-3-piperidyl)amino]phenyl]-4-piperidyl]methyl]-2,8-diazaspiro[4.5]decane-2-carboxylate (110 mg, 195.66 μmol, 46.59% yield, 96% purity) as black solid. MS(M+H)⁺=540.3.

### Step 6. Synthesis of 3-[4-[4-(2,8-diazaspiro[4.5]decan-8-ylmethyl)-1-piperidyl]anilino]piperidine-2,6-dione

To a solution of tert-butyl 8-[[1-[4-[(2,6-dioxo-3-piperidyl)amino]phenyl]-4-piperidyl]methyl]-2,8-diazaspiro[4.5]decane-2-carboxylate (110 mg, 203.81 μmol) in 1,4-dioxane (2 mL) was added HCl/dioxane (4 M, 2 mL). The mixture was stirred at 15° C. for 2 hr. LCMS showed a peak (94%) with the mass of desired product. The reaction mixture was concentrated under reduced pressure to afford 3-[4-[4-(2,8-diazaspiro[4.5]decan-8-ylmethyl)-1-piperidyl]anilino]piperidine-2,6-dione (90 mg, 189.05 μmol, 92.76% yield, HCl) as black solid. MS(M+H)⁺=440.3.

### Step 7. Synthesis of N-(2-chlorophenyl)-4-[[2-[4-[8-[[1-[4-[(2,6-dioxo-3-piperidyl)amino]phenyl]-4-piperidyl]methyl]-2,8-diazaspiro[4.5]decane-2-carbonyl]anilino]-5-fluoro-pyrimidin-4-yl]amino]benzamide (Compound 33)

To a solution of 3-[4-[4-(2,8-diazaspiro[4.5]decan-8-ylmethyl)-1-piperidyl]anilino]piperidine-2,6-dione (90 mg, 204.74 μmol) and 4-[[4-[4-[(2-chlorophenyl)carbamoyl]anilino]-5-fluoro-pyrimidin-2-yl]amino]benzoic acid (78.27 mg, 163.79 μmol) in DMF (1 mL) were added HATU (116.77 mg, 307.10 mol) and DIPEA (79.38 mg, 614.21 μmol, 106.98 μL). The resulting mixture was stirred at 15° C. for 16 hr. LCMS showed a peak (57%) with the mass of desired product. The reaction mixture was poured into water (20 mL) and filtered to get the crude product as a solid. The crude product was purified by prep-HPLC (column: Phenomenex luna C18 150*25 mm*10 um; mobile phase: [water(TFA)-ACN]; gradient: 21%-51% B over 10 min) and lyophilized. Followed by further purification of prep-HPLC (column: Waters Xbridge 150*25 mm*5 um; mobile phase: [water(NH₄HCO₃)-ACN]; gradient: 28%-58% B over 10 min) and lyophilized to afford N-(2-chlorophenyl)-4-[[2-[4-[8-[[1-[4-[(2,6-dioxo-3-piperidyl)amino]phenyl]-4-piperidyl]methyl]-2,8-diazaspiro[4.5]decane-2-carbonyl]anilino]-5-fluoro-pyrimidin-4-yl]amino]benzamide (15.6 mg, 16.13 μmol, 7.88% yield, 93% purity) as white solid. MS(M+H)⁺=899.4.

¹H NMR (400 MHz, DMSO-d₆) δ=10.76 (br s, 1H), 9.94 (s, 1H), 9.75 (s, 1H), 9.59 (br d, J=4.9 Hz, 1H), 8.24 (br d, J=2.0 Hz, 1H), 8.10-8.00 (m, 4H), 7.75 (br dd, J=7.3, 10.8 Hz, 2H), 7.69-7.62 (m, 1H), 7.57 (br d, J=7.9 Hz, 1H), 7.53-7.37 (m, 3H), 7.35-7.26 (m, 1H), 6.83-6.69 (m, 2H), 6.67-6.52 (m, 2H), 5.44-5.30 (m, 1H), 4.25-4.15 (m, 1H), 3.61-3.47 (m, 2H), 2.27-1.99 (m, 9H), 1.94-1.64 (m, 9H), 1.62-1.38 (m, 6H), 1.31-1.04 (m, 3H).

### Example 34. Synthesis of N-(2-chlorophenyl)-4-((2-((4-(3-((5-(4-((2,6-dioxopiperidin-3-yl)amino)phenyl)hexahydropyrrolo[3,4-c]pyrrol-2(1H)-yl)methyl)azetidine-1-carbonyl)phenyl)amino)-5-fluoropyrimidin-4-yl)amino)benzamide (Compound 34)

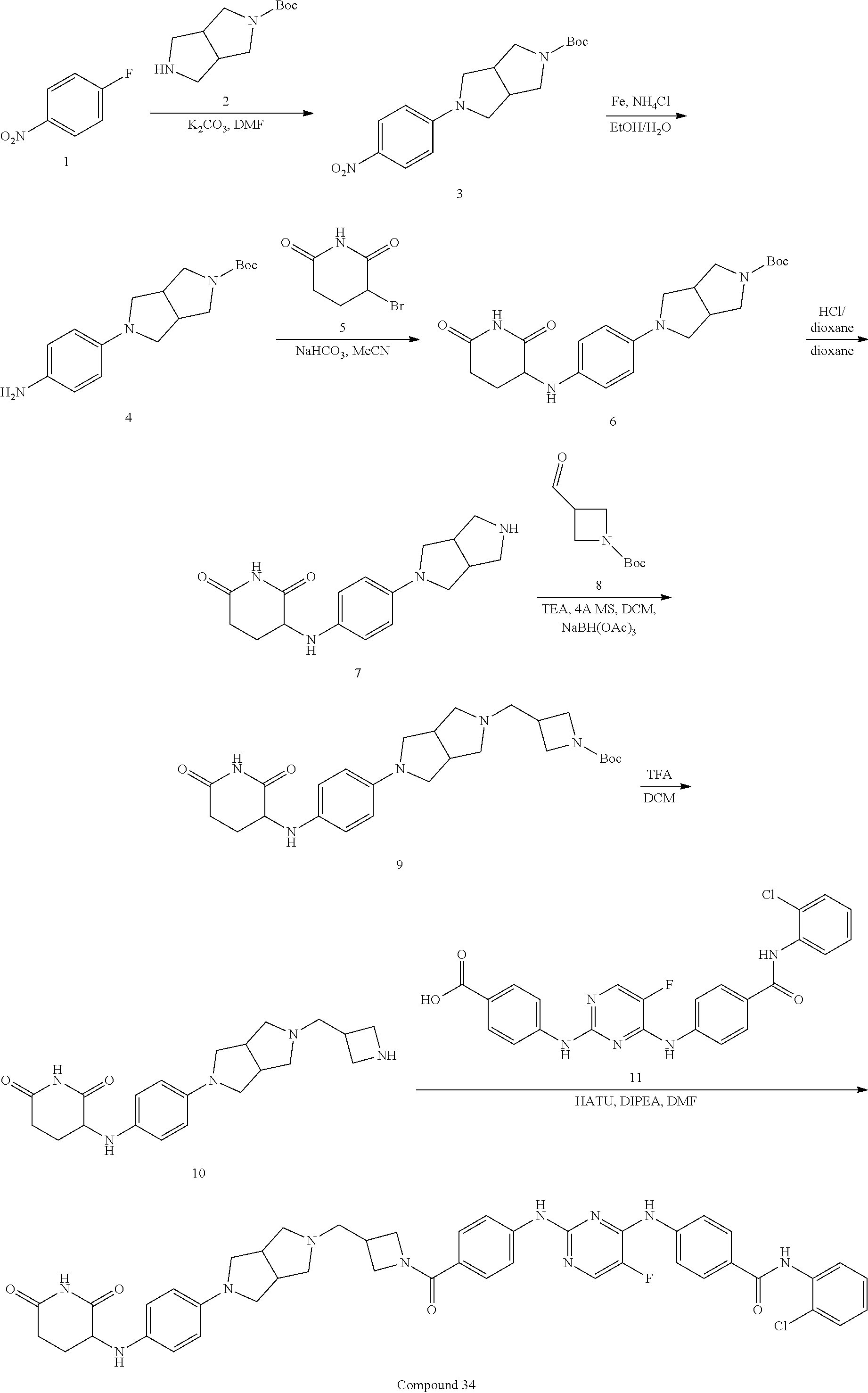

### Step 1. Synthesis of tert-butyl 5-(4-nitrophenyl)hexahydropyrrolo[3,4-c]pyrrole-2(1H)-carboxylate (3)

To a solution of 1-fluoro-4-nitrobenzene (1 g, 7.09 mmol) and tert-butyl hexahydropyrrolo[3,4-c]pyrrole-2(1H)-carboxylate (1.65 g, 7.80 mmol) in DMF (30 mL) was added K₂CO₃ (2.94 g, 21.26 mmol) at 20° C. The mixture was stirred at 40° C. for 16 h. LCMS showed the starting material was consumed completely and 96% peak with desired mass. The reaction mixture was diluted with H₂O (60 mL) and extracted with EtOAc (30 mL×3). The combined organic layers were washed with brine (30 mL×3), dried over Na₂SO₄ and concentrated in vacuum to afford tert-butyl 5-(4-nitrophenyl)hexahydropyrrolo[3,4-c]pyrrole-2(1H)-carboxylate (2.30 g, 6.76 mmol, 95.40% yield, 98% purity) as a yellow solid. MS(M+H)⁺=334.4.

### Step 2. Synthesis of tert-butyl 5-(4-aminophenyl)hexahydropyrrolo[3,4-c]pyrrole-2(1H)-carboxylate (4)

To a solution of tert-butyl 5-(4-nitrophenyl)hexahydropyrrolo[3,4-c]pyrrole-2(1H)-carboxylate (2.3 g, 6.90 mmol) in EtOH (20 mL) and H₂O (20 mL) were added Fe (1.93 g, 34.50 mmol) and NH₄Cl (1.48 g, 27.60 mmol) at 20° C. The mixture was stirred at 80° C. for 2 h. LCMS showed the starting material was consumed completely and 81% peak with desired mass. The reaction mixture was concentrated in vacuum to give a residue. The residue was diluted with H₂O (100 mL) and adjusted to pH=10 with Na₂CO₃, then extracted with EtOAc (40 mL×3). The combined organic layers were dried over Na₂SO₄ and concentrated in vacuum to afford tert-butyl 5-(4-aminophenyl)hexahydropyrrolo[3,4-c]pyrrole-2(1H)-carboxylate (2 g, 6.39 mmol, 92.68% yield, 97% purity) as a light yellow solid. MS(M+H)⁺=304.4.

### Step 3. Synthesis of tert-butyl 5-(4-((2,6-dioxopiperidin-3-yl)amino)phenyl)hexahydropyrrolo[3,4-c]pyrrole-2(1H)-carboxylate (6)

To a solution of tert-butyl 5-(4-aminophenyl)hexahydropyrrolo[3,4-c]pyrrole-2(1H)-carboxylate (1 g, 3.30 mmol) and 3-bromopiperidine-2,6-dione (1.14 g, 5.93 mmol) in MeCN (10 mL) was added NaHCO₃ (1.38 g, 16.48 mmol) at 20° C. The mixture was stirred at 80° C. for 16 h. LCMS showed the starting material was consumed completely and 58% peak with desired mass (Rₜ=0.700 min). The reaction mixture was filtered and washed with EtOAc (20 mL). The filtrate was concentrated in vacuum to give a residue, which was purified by flash silica gel chromatography (ISCO®; 20 g SepaFlash® Silica Flash Column, eluent of 80% EtOAc/petroleum ether, gradient @ 100 mL/min) to afford tert-butyl 5-(4-((2,6-dioxopiperidin-3-yl)amino)phenyl)hexahydropyrrolo[3,4-c]pyrrole-2(1H)-carboxylate (658 mg, 1.59 mmol, 48.16% yield) as an off-white solid. MS(M+H)⁺=415.2.

### Step 4. Synthesis of 3-((4-(hexahydropyrrolo[3,4-c]pyrrol-2(1H)-yl)phenyl)amino)piperidine-2,6-dione (7)

To a solution of tert-butyl 5-(4-((2,6-dioxopiperidin-3-yl)amino)phenyl)hexahydropyrrolo[3,4-c]pyrrole-2(1H)-carboxylate (558 mg, 1.35 mmol) in dioxane (2 mL) was added HCl/dioxane (4 M, 10 mL) at 20° C. The mixture was stirred at 20° C. for 1 h. LCMS showed the starting material was consumed completely and 97% peak with desired mass. The reaction mixture was concentrated in vacuum to afford 3-((4-(hexahydropyrrolo[3,4-c]pyrrol-2(1H)-yl)phenyl)amino)piperidine-2,6-dione (473 mg, crude, HCl) as a green solid. MS(M+H)⁺=315.2.

### Step 5. Synthesis of tert-butyl 3-((5-(4-((2,6-dioxopiperidin-3-yl)amino)phenyl)hexahydropyrrolo[3,4-c]pyrrol-2(1H)-yl)methyl)azetidine-1-carboxylate (9)

To a solution of 3-((4-(hexahydropyrrolo[3,4-c]pyrrol-2(1H)-yl)phenyl)amino)piperidine-2,6-dione (473 mg, 1.35 mmol, HCl) in DCM (7 mL) were added TEA (409.26 mg, 4.04 mmol), 4 Å MS (100 mg) and tert-butyl 3-formylazetidine-1-carboxylate (249.71 mg, 1.35 mmol). After stirring at 20° C. for 10 min, NaBH(OAc)₃ (857.21 mg, 4.04 mmol) was slowly added. The resulting mixture was stirred at 20° C. for 16 h. LCMS showed the starting material was consumed completely and 67% peak with desired mass (Rₜ=0.583 min). The reaction mixture was filtered and the filtrate was concentrated in vacuum to give a residue. The residue was diluted with H₂O (40 mL) and extracted with EtOAc (20 mL×3). The combined organic layers were dried over Na₂SO₄ and concentrated in vacuum to afford the crude product. The crude product was purified by flash silica gel chromatography (ISCO®; 20 g SepaFlash® Silica Flash Column, eluent of 50-100% EtOAc in petroleum, and then eluted with to 0~20% MeOH in DCM, gradient @ 100 mL/min) to afford the crude product. The crude product was further purified by prep-HPLC (column: Phenomenex luna C18 150*40 mm*15 um; mobile phase: [water(TFA)-ACN]; gradient: 10%-40% B over 10 min) and lyophilized to afford tert-butyl 3-((5-(4-((2,6-dioxopiperidin-3-yl)amino)phenyl)hexahydropyrrolo[3,4-c]pyrrol-2(1H)-yl)methyl)azetidine-1-carboxylate (340 mg, 563.23 μmol, 41.78% yield, 99% purity, TFA) as a black purple solid. MS(M+H)⁺=484.3.

### Step 6. Synthesis of 3-((4-(5-(azetidin-3-ylmethyl)hexahydropyrrolo[3,4-c]pyrrol-2(1H)-yl)phenyl) aminol)piperidine-2,6-dione (10)

To a solution of tert-butyl 3-((5-(4-((2,6-dioxopiperidin-3-yl)amino)phenyl)hexahydropyrrolo[3,4-c]pyrrol-2(1H)-yl)methyl)azetidine-1-carboxylate (340 mg, 568.92 μmol, TFA) in DCM (3 mL) was added TFA (778.42 mg, 6.83 mmol). The mixture was stirred at 20° C. for 1 h. LCMS showed the starting material was consumed completely and desired mass (Rₜ=0.150 min). The reaction mixture was concentrated in vacuum to afford 3-((4-(5-(azetidin-3-ylmethyl)hexahydropyrrolo[3,4-c]pyrrol-2(1H)-yl)phenyl)amino)piperidine-2,6-dione (250 mg, crude, TFA) as a black blue oil. MS(M+H)⁺=384.3.

### Step 7. Synthesis of N-(2-chlorophenyl)-4-((2-((4-(3-((5-(4-((2,6-dioxopiperidin-3-yl)amino)phenyl)hexahydropyrrolo[3,4-c]pyrrol-2(1H)-yl)methyl)azetidine-1-carbonyl)phenyl)amino)-5-fluoropyrimidin-4-yl)amino)benzamide (Compound 34)

To a solution of 4-((4-((4-((2-chlorophenyl)carbamoyl)phenyl)amino)-5-fluoropyrimidin-2-yl)amino)benzoic acid (216.12 mg, 452.25 μmol) in DMF (2 mL) was added HATU (171.96 mg, 452.25 μmol) and DIPEA (292.25 mg, 2.26 mmol, 393.87 μL) at 20° C. After stirring for 10 min, a solution of 3-((4-(5-(azetidin-3-ylmethyl)hexahydropyrrolo[3,4-c]pyrrol-2(1H)-yl)phenyl)amino)piperidine-2,6-dione (225 mg, 452.25 μmol, TFA) and DIPEA (667.80 mg, 5.17 mmol) in DMF (2 mL) was added at 20° C. The resulting mixture was stirred at 20° C. for 1 h. LCMS showed the starting material was consumed completely and desired mass (Rₜ=0.677 min). The reaction mixture was diluted with H₂O (20 mL) and EtOAc (30 ml). The mixture was filtered. The filter cake was washed by EtOH (20 mL) and concentrated in vacuum to afford the crude product, which was purified by prep-HPLC (column: Phenomenex luna C18 150*40 mm*15 um; mobile phase: [water(TFA)-ACN]; gradient: 15%-45% B over 10 min) and lyophilized to afford N-(2-chlorophenyl)-4-((2-((4-(3-((5-(4-((2,6-dioxopiperidin-3-yl)amino)phenyl)hexahydropyrrolo[3,4-c]pyrrol-2(1H)-yl)methyl)azetidine-1-carbonyl)phenyl)amino)-5-fluoropyrimidin-4-yl)amino) benzamide (111.4 mg, 86.46 μmol, 19.12% yield, 92% purity, 3TFA) as a purple solid. MS(M+H)⁺=843.1.

¹H NMR (400 MHz, DMSO-d₆) δ=10.85 (s, 1H), 9.96 (s, 1H), 9.78 (s, 1H), 9.67 (s, 2H), 8.25 (d, J=3.5 Hz, 1H), 8.05-7.95 (m, 4H), 7.77 (d, J=8.7 Hz, 2H), 7.64 (d, J=7.7 Hz, 1H), 7.60-7.52 (m, 3H), 7.44-7.36 (m, 1H), 7.34-7.25 (m, 1H), 6.80-6.70 (m, 2H), 6.68-6.57 (m, 2H), 4.57-4.43 (m, 2H), 4.28-4.22 (m, 1H), 4.19-4.10 (m, 2H), 3.89-3.89 (m 2H), 3.59-3.40 (m, 3H), 3.40-3.35 (m, 1H), 3.33-3.24 (m, 1H), 3.24-3.12 (m, 2H), 3.09-2.93 (m, 2H), 2.90-2.81 (m, 1H), 2.80-2.72 (m, 1H), 2.64-2.53 (m, 2H), 2.09-2.00 (m, 1H), 1.93-1.77 (m, 1H).

### Example 35. Synthesis of N-(2-chlorophenyl)-4-((2-((4-(5-((1-(4-((2,6-dioxopiperidin-3-yl)amino)phenyl)azetidin-3-yl)methyl)octahydropyrrolo[3,4-c]pyrrole-2-carbonyl)phenyl)amino)-5-fluoro pyrimidin-4-yl)amino)benzamide (Compound 35)

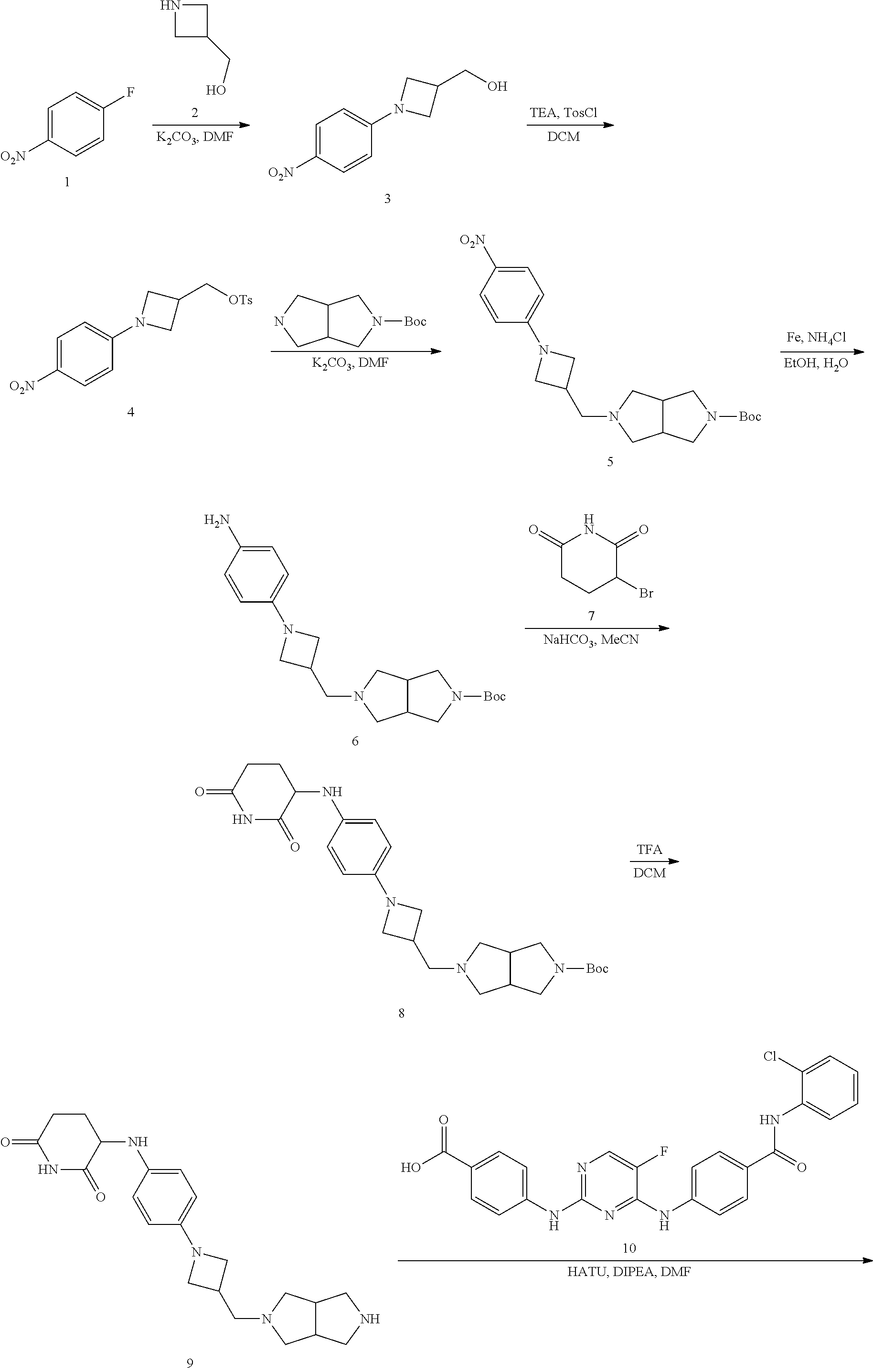

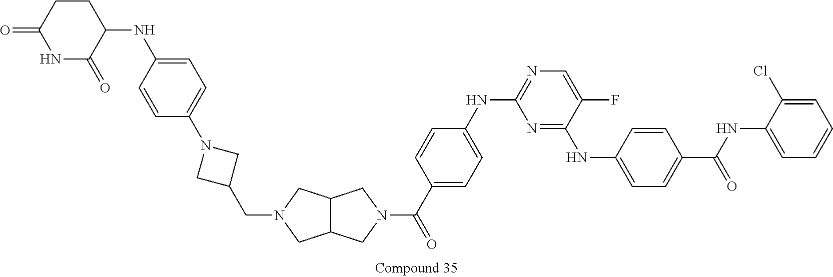 [SMILES: O=C1CCC(Nc2ccc(N3CC(CN4CC5CN(C(=O)c6ccc(Nc7ncc(F)c(Nc8ccc(C(=O)Nc9ccccc9Cl)cc8)n7)cc6)CC5C4)C3)cc2)C(=O)N1]

### Step 1. Synthesis of (1-(4-nitrophenyl)azetidin-3-yl)methanol (3)

To a solution of 1-fluoro-4-nitrobenzene (2.5 g, 17.72 mmol, 1.88 mL) and azetidin-3-ylmethanol (2.19 g, 17.72 mmol, HCl) in DMF (30 mL) was added K₂CO₃ (7.35 g, 53.15 mmol) at 20′° C. The reaction mixture was stirred at 40° C. for 16 h. LCMS showed 79% peak with desired mass (Rt=0.621 min) was detected. The reaction mixture was diluted with water (60 mL) and extracted with EtOAc (30 mL×3). The combined organic layers were washed with brine (30 mL), dried over Na₂SO₄ and concentrated in vacuum and then purified by flash silica gel chromatography (40 g SepaFlash® Silica Flash Column, eluent of 10-50% EtOAc/petroleum ether, gradient @ 100 mL/min) to afford (1-(4-nitrophenyl)azetidin-3-yl)methanol (3.4 g, 16.17 mmol, 91.24% yield, 99% purity) as a red solid. MS(M+H)⁺=209.5.

### Step 2. Synthesis of (1-(4-nitrophenyl)azetidin-3-yl)methyl 4-methylbenzenesulfonate (4)

To a solution of (1-(4-nitrophenyl)azetidin-3-yl)methanol (2 g, 9.61 mmol) in DCM (20 mL) were added TEA (2.92 g, 28.82 mmol, 4.01 mL) and TosCl (2.75 g, 14.41 mmol) at 20° C. The mixture was stirred at 20° C. for 16 h. LCMS showed the starting material was consumed completely and 87% peak with desired mass (Rt=0.851 min) was detected. The reaction mixture was concentrated in vacuum and then purified by flash silica gel chromatography (20 g SepaFlash® Silica Flash Column, eluent of 10~30% EtOAc/petroleum ether, gradient @ 100 mL/min) to afford [1-(4-nitrophenyl)azetidin-3-yl]methyl 4-methylbenzenesulfonate (3.02 g, 8.17 mmol, 85.02% yield, 98% purity) as a yellow solid. MS(M+H)⁺=363.1.

### Step 3. Synthesis of tert-butyl 5-((1-(4-nitrophenyl)azetidin-3-yl)methyl)hexahydropyrrolo[3,4-c]pyrrole-2(1H)-carboxylate (5)

To a solution of (1-(4-nitrophenyl)azetidin-3-yl)methyl 4-methylbenzenesulfonate (1.4 g, 3.86 mmol) and tert-butyl hexahydropyrrolo[3,4-c]pyrrole-2(1H)-carboxylate (820.10 mg, 3.86 mmol) in DMF (20 mL) was added K₂CO₃ (1.60 g, 11.59 mmol) at 20° C. The mixture was stirred at 40° C. for 16 h. LCMS showed (1-(4-nitrophenyl)azetidin-3-yl)methyl 4-methylbenzenesulfonate remained and the desired mass was detected. The reaction mixture was diluted with water (60 mL) and extracted with EtOAc (30 mL×3). The combined organic layer was washed with brine (30 mL×3), dried over Na₂SO₄ and filtered. The filtrate was concentrated in vacuum and then purified by flash silica gel chromatography (12 g SepaFlash® Silica Flash Column, eluent of 0-40% EtOAc/petroleum ether, gradient @ 100 mL/min) to afford tert-butyl 5-((1-(4-nitrophenyl)azetidin-3-yl)methyl)hexahydropyrrolo[3,4-c]pyrrole-2(1H)-carboxylate (900 mg, 2.24 mmol, 57.88% yield) as a yellow oil. MS(M+H)⁺=403.2.

### Step 4. Synthesis of tert-butyl 5-((1-(4-aminophenyl)azetidin-3-yl)methyl)hexahydropyrrolo[3,4-c]pyrrole-2(1H)-carboxylate (6)

To a solution of tert-butyl 5-((1-(4-nitrophenyl)azetidin-3-yl)methyl)hexahydropyrrolo[3,4-c]pyrrole-2(1H)-carboxylate (900 mg, 2.24 mmol) in EtOH (10 mL) and H₂O (10 mL) were added Fe (499.50 mg, 8.94 mmol) and NH₄Cl (478.45 mg, 8.94 mmol) at 20° C. The mixture was stirred at 80° C. for 1.5 h. LCMS showed the starting material was consumed completely and 73% peak with desired mass. The reaction mixture was concentrated in vacuum to give a residue. The residue was diluted with water (60 mL) and adjusted to pH=10 with saturation Na₂CO₃, extracted with EtOAc (40 mL×3). The combined organic layers were dried over Na₂SO₄ and concentrated in vacuum to afford tert-butyl 2-[[1-(4-aminophenyl)azetidin-3-yl]methyl]-1,3,3a,4,6,6a-hexahydropyrrolo[3,4-c]pyrrole-5-carboxylate (800 mg, 2.15 mmol, 96.04% yield) as a yellow solid. MS(M+H)⁺=373.4.

### Step 5. Synthesis of tert-butyl 5-((1-(4-((2,6-dioxopiperidin-3-yl)amino)phenyl)azetidin-3-yl)methyl) hexahydropyrrolo[3,4-c]pyrrole-2(1H)-carboxylate (8)

To a solution of tert-butyl 5-((1-(4-aminophenyl)azetidin-3-yl)methyl)hexahydropyrrolo[3,4-c]pyrrole-2(1H)-carboxylate (800 mg, 2.15 mmol) and 3-bromopiperidine-2,6-dione (288.66 mg, 1.50 mmol) in MeCN (3 mL) was added NaHCO₃ (541.25 mg, 6.44 mmol) at 20° C. The mixture was stirred at 80° C. for 2 h. LCMS showed all the starting material was consumed completely and 27% peak with desired mass was detected. The reaction mixture was filtered and washed with EtOAc:MeOH:THF=1:2:1 (40 mL). The filtrate was concentrated in vacuum and then purified by flash silica gel chromatography (12 g SepaFlash® Silica Flash Column, eluent of 30-100% EtOAc/petroleum ether to 0~20% MeOH/DCM, gradient @ 100 mL/min). The crude product was further purified by prep-HPLC (column: Phenomenex luna C18 150*40 mm*15 um; mobile phase: [water(TFA)-ACN]; gradient: 5%-35% B over 10 min) and lyophilized to afford tert-butyl 5-((1-(4-((2,6-dioxopiperidin-3-yl)amino)phenyl)azetidin-3-yl)methyl)hexahydropyrrolo[3,4-c]pyrrole-2(1H)-carboxylate (370 mg, 594.35 μmol, 27.67% yield, 96% purity, TFA) as a purple solid. MS(M+H)⁺=484.3.

### Step 6. Synthesis of 3-((4-(3-((hexahydropyrrolo[3,4-c]pyrrol-2(1H)-yl)methyl)azetidin-1-yl)phenyl) amino)piperidine-2,6-dione (9)

To a solution of tert-butyl 5-((1-(4-((2,6-dioxopiperidin-3-yl)amino)phenyl)azetidin-3-yl)methyl)hexahydropyrrolo[3,4-c]pyrrole-2(1H)-carboxylate (0.2 g, 334.66 μmol, TFA) in DCM (1.5 mL) was added TFA (767.50 mg, 6.73 mmol, 0.5 mL) at 0° C. and the mixture was stirred at 0° C. for 1 h. LCMS showed 95% the desired mass. The mixture was concentrated under reduced pressure (0° C.) to afford 3-((4-(3-((hexahydropyrrolo[3,4-c]pyrrol-2(1H)-yl)methyl)azetidin-1-yl)phenyl)amino)piperidine-2,6-dione (170 mg, crude, TFA) as a yellow oil. MS(M+H)⁺=384.3.

### Step 7. Synthesis of N-(2-chlorophenyl)-4-((2-((4-(5-((1-(4-((2,6-dioxopiperidin-3-yl)amino)phenyl)azetidin-3-yl)methyl)octahydropyrrolo[3,4-c]pyrrole-2-carbonyl)phenyl)amino)-5-fluoropyrimidin-4-yl)amino)benzamide (Compound 35)

To a solution of 4-((4-((4-((2-chlorophenyl)carbamoyl)phenyl)amino)-5-fluoropyrimidin-2-yl)amino)benzoic acid (160 mg, 334.82 mol) and HATU (153 mg, 402.39 μmol) in DMF (1.5 mL) was added DIPEA (432.73 mg, 3.35 mmol, 583.19 μL) and the mixture was stirred at 20° C. for 15 min. Then a solution of 3-((4-(3-((hexahydropyrrolo[3,4-c]pyrrol-2(1H)-yl)methyl)azetidin-1-yl)phenyl)amino)piperidine-2,6-dione (170 mg, 341.70 μmol, TFA) in DMF (1.5 mL) at 0° C. was added and the mixture was stirred at 0° C. for 1 h. LCMS showed 58% of the desired mass. The mixture was diluted with water (50 mL) and then filtered. The filter cake was washed with water (30 mL). The filter cake was collected and the solvent was removed under reduced pressure and then purified by prep-TLC (DCM:MeOH=7:1). The crude product was purified by prep-HPLC (column: Phenomenex luna C18 150*25 mm*10 um; mobile phase: [water (TFA)-ACN]; gradient: 18%-48% B over 9 min) and lyophilized to afford N-(2-chlorophenyl)-4-((2-((4-(5-((1-(4-((2,6-dioxopiperidin-3-yl)amino)phenyl)azetidin-3-yl)methyl)octahydropyrrolo[3,4-c]pyrrole-2-carbonyl)phenyl)amino)-5-fluoropyrimidin-4-yl)amino)benzamide (64.5 mg, 47.15 μmol, 56.49% yield, 95% purity, 4TFA) as a gray solid. MS(M+H)⁺=843.3.

¹H NMR (400 MHz, DMSO-d₆) δ=11.00-10.82 (m, 1H), 10.05-9.87 (m, 2H), 9.78 (s, 1H), 9.64 (s, 1H), 8.24 (d, J=3.5 Hz, 1H), 8.05-7.98 (m, 4H), 7.76 (d, J=8.7 Hz, 2H), 7.66-7.61 (m, 1H), 7.58-7.53 (m, 1H), 7.49-7.44 (m, 2H), 7.41-7.36 (m, 1H), 7.31-7.26 (m, 1H), 6.84-6.66 (m, 2H), 6.62-6.47 (m, 2H), 4.34-4.24 (m, 1H), 4.04-3.99 (m, 1H), 3.76-3.56 (m, 6H), 3.52-3.45 (m, 2H), 3.12-2.97 (m, 3H), 2.77-2.69 (m, 1H), 2.62-2.52 (m, 5H), 2.46-2.35 (m, 2H), 2.10-1.97 (m, 1H), 1.93-1.77 (m, 1H).

### Example 36. Synthesis of N-(2-chlorophenyl)-4-((2-((4-((2-(1-(4-((2,6-dioxopiperidin-3-yl)amino)phenyl)azetidin-3-yl)-1,2,3,4-tetrahydroisoquinolin-6-yl)carbamoyl)phenyl)amino)-5-fluoropyrimidin-4-yl)amino)benzamide (Compound 36)

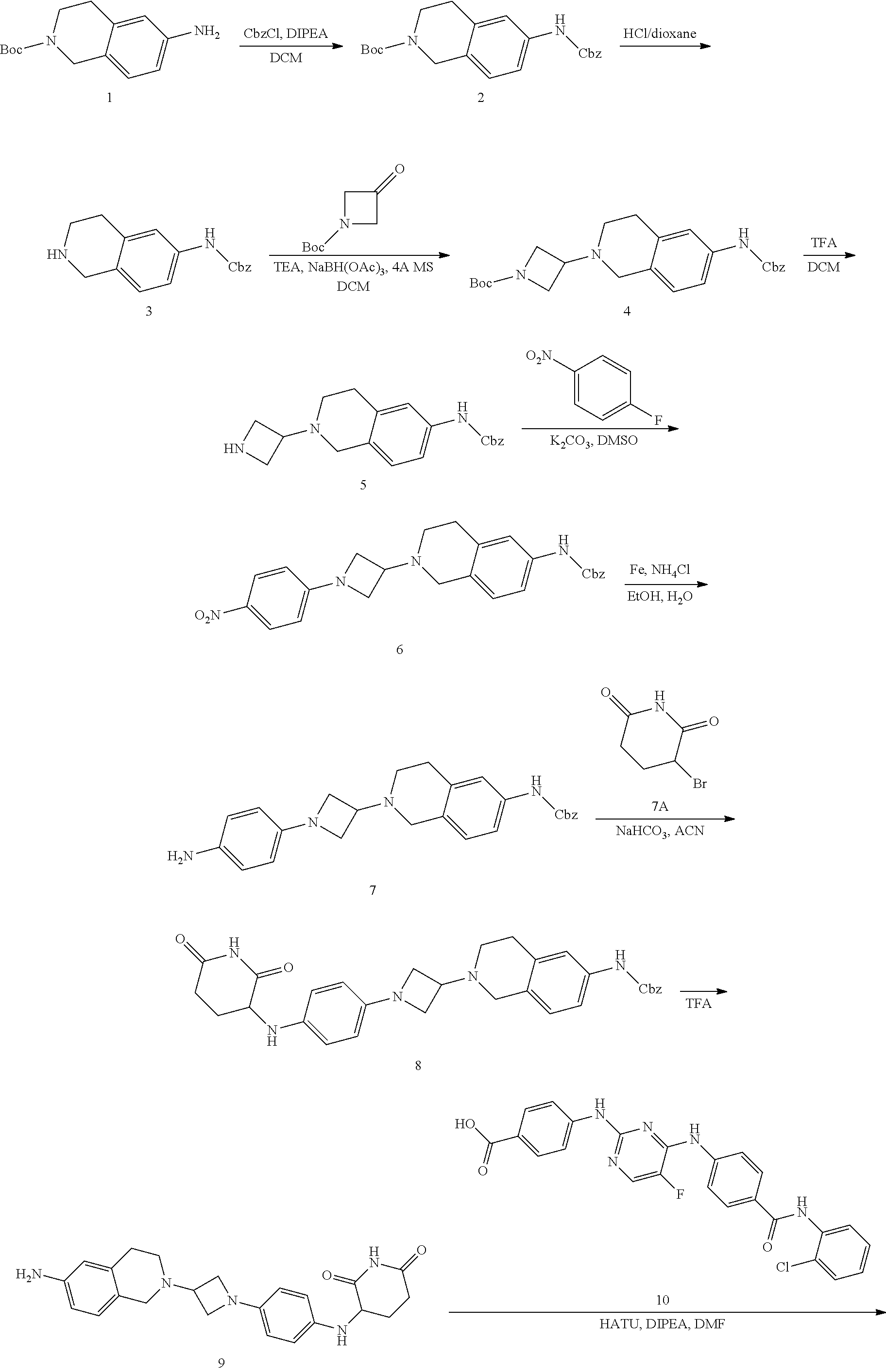

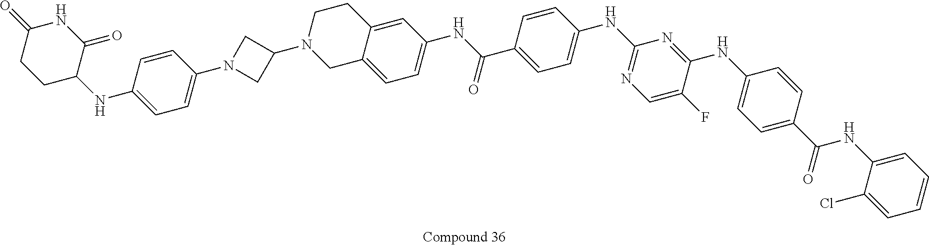 [SMILES: O=C1CCC(Nc2ccc(N3CC(N4CCc5cc(NC(=O)c6ccc(Nc7ncc(F)c(Nc8ccc(C(=O)Nc9ccccc9Cl)cc8)n7)cc6)ccc5C4)C3)cc2)C(=O)N1]

### Step 1. Synthesis of tert-butyl 6-(((benzyloxy)carbonyl)amino)-3,4-dihydroisoquinoline-2(1H)-carboxylate (2)

To a solution of tert-butyl 6-amino-3,4-dihydroisoquinoline-2(1H)-carboxylate (2 g, 8.05 mmol) in DCM (30 mL) was added DIPEA (3.12 g, 24.16 mmol, 4.21 mL) at 20° C., then CbzCl (1.65 g, 9.66 mmol, 1.38 mL) was added drop-wise at 0° C. The resulting mixture was stirred at 20° C. for 16 h. LCMS showed the starting material was consumed completely and 82% peak with desired mass was detected. The reaction mixture was concentrated in vacuum to give a residue. The residue was purified by flash silica gel chromatography (ISCO®; 40 g SepaFlash® Silica Flash Column, eluent of 0~20% EtOAc/petroleum ether, gradient @ 100 mL/min) to afford tert-butyl 6-(((benzyloxy)carbonyl)amino)-3,4-dihydroisoquinoline-2(1H)-carboxylate (3 g, 7.84 mmol, 97.39% yield) as a colorless oil. MS(M+H)⁺=383.4.

### Step 2. Synthesis of benzyl (1,2,3,4-tetrahydroisoquinolin-6-yl)carbamate (3)

A solution of tert-butyl 6-(((benzyloxy)carbonyl)amino)-3,4-dihydroisoquinoline-2(1H)-carboxylate (3 g, 7.84 mmol) in HCl/dioxane (4 M, 30 mL) was stirred at 20° C. for 1 h. LCMS showed the starting material was consumed completely and 68% peak with desired mass (Rₜ=0.667 min) was detected. The reaction mixture was concentrated in vacuum to afford benzyl (1,2,3,4-tetrahydroisoquinolin-6-yl)carbamate (2.5 g, 5.33 mmol, 67.98% yield, 68% purity, HCl) as a white solid. MS(M+H)⁺=283.2.

### Step 3. Synthesis of tert-butyl 3-(6-(((benzyloxy)carbonyl)amino)-3,4-dihydroisoquinolin-2(1H)-yl)azetidine-1-carboxylate (4)

To a solution of benzyl (1,2,3,4-tetrahydroisoquinolin-6-yl)carbamate (2.4 g, 7.53 mmol, HCl) in DCM (40 mL) were added TEA (2.29 g, 22.58 mmol, 3.14 mL), 4 Å MS (200 mg) and tert-butyl 3-oxoazetidine-1-carboxylate (1.42 g, 8.28 mmol) at 20° C. After stirring for 20 min, NaBH(OAc)₃ (4.79 g, 22.58 mmol) was slowly added at 20° C. The mixture was stirred at 20° C. for 16 h. LCMS showed 8% peak of benzyl (1,2,3,4-tetrahydroisoquinolin-6-yl)carbamate was remained and 62% peak with desired mass (Rₜ=0.796 min) was detected. The reaction mixture was filtered and the filtrate was concentrated in vacuum to give a residue. The residue was diluted with H₂O (50 mL) and extracted with EtOAc (30 mL×3). The combined organic layers were washed with NaHCO₃ (30 mL×3), dried over Na₂SO₄ and concentrated in vacuum to afford the crude product, which was purified by flash silica gel chromatography (ISCO®; 40 g SepaFlash® Silica Flash Column, eluent of 0-40% EtOAc/petroleum ether, gradient @ 100 mL/min) to afford tert-butyl 3-(6-(((benzyloxy)carbonyl)amino)-3,4-dihydroisoquinolin-2(1H)-yl)azetidine-1-carboxylate (2.7 g, 6.17 mmol, 81.97% yield) as a white solid. MS(M+H)⁺=438.3.

### Step 4. Synthesis of benzyl (2-(azetidin-3-yl)-1,2,3,4-tetrahydroisoquinolin-6-yl)carbamate (5)

To a solution of tert-butyl 3-(6-(((benzyloxy)carbonyl)amino)-3,4-dihydroisoquinolin-2(1H)-yl)azetidine-1-carboxylate (2.7 g, 6.17 mmol) in DCM (20 mL) was added TFA (8.44 g, 74.05 mmol, 5.50 mL) at 20° C. The mixture was stirred at 20° C. for 2 h. LCMS showed the starting material was consumed completely and 93% peak with desired mass was detected. The reaction mixture was concentrated in vacuum to afford benzyl (2-(azetidin-3-yl)-1,2,3,4-tetrahydroisoquinolin-6-yl)carbamate (2.6 g, crude, TFA) as a white solid. MS(M+H)⁺=338.2.

### Step 5. Synthesis of benzyl (2-(1-(4-nitrophenyl)azetidin-3-yl)-1,2,3,4-tetrahydroisoquinolin-6-yl)carbamate (6)

To a solution of benzyl (2-(azetidin-3-yl)-1,2,3,4-tetrahydroisoquinolin-6-yl)carbamate (2.6 g, 5.76 mmol, TFA) and 1-fluoro-4-nitrobenzene (893.91 mg, 6.34 mmol) in DMSO (40 mL) was added K₂CO₃ (4.78 g, 34.56 mmol) at 20° C. The mixture was stirred at 40° C. for 16 h. LCMS showed 11% of benzyl (2-(azetidin-3-yl)-1,2,3,4-tetrahydroisoquinolin-6-yl)carbamate was remained and 87% peak with desired mass was detected. The reaction mixture was diluted with H₂O (60 mL) and extracted with EtOAc (30 mL×3). The combined organic layers were washed with brine (30 mL×3), dried over Na₂SO₄ and concentrated in vacuum to give a residue. The residue was purified by flash silica gel chromatography (ISCO®; 20 g SepaFlash® Silica Flash Column, eluent of 0~50% EtOAc/petroleum ether, gradient @ 200 mL/min) to afford benzyl (2-(1-(4-nitrophenyl)azetidin-3-yl)-1,2,3,4-tetrahydroisoquinolin-6-yl)carbamate (2.3 g, 5.02 mmol, 87.10% yield) as a yellow solid. MS(M+H)⁺=459.3.

### Step 6. Synthesis of benzyl (2-(1-(4-aminophenyl)azetidin-3-yl)-1,2,3,4-tetrahydroisoquinolin-6-yl)carbamate (7)

To a solution of benzyl (2-(1-(4-nitrophenyl)azetidin-3-yl)-1,2,3,4-tetrahydroisoquinolin-6-yl)carbamate (2.3 g, 5.02 mmol) in EtOH (20 mL) and H₂O (20 mL) were added Fe (1.40 g, 25.08 mmol) and NH₄Cl (1.07 g, 20.07 mmol) at 20° C. The mixture was stirred at 80° C. for 3 h. LCMS showed benzyl (2-(1-(4-nitrophenyl)azetidin-3-yl)-1,2,3,4-tetrahydroisoquinolin-6-yl)carbamate was consumed completely and 94% peak with desired mass was detected. The reaction mixture was filtered and the filtrate concentrated in vacuum to give a residue. The residue was diluted with H₂O (60 mL) and adjusted to pH=10 with Na₂CO₃, extracted with EtOAc (30 mL×3). The combined organic layers were dried over Na₂SO₄ and concentrated in vacuum to afford benzyl (2-(1-(4-aminophenyl)azetidin-3-yl)-1,2,3,4-tetrahydroisoquinolin-6-yl)carbamate (820 mg, 1.84 mmol, 84.23% yield, 96% purity) as a yellow solid. MS(M+H)⁺=429.2.

### Step 7. Synthesis of benzyl (2-(1-(4-((2,6-dioxopiperidin-3-yl)amino)phenyl)azetidin-3-yl)-1,2,3,4-tetrahydroisoquinolin-6-yl)carbamate (8)

To a solution of benzyl (2-(1-(4-aminophenyl)azetidin-3-yl)-1,2,3,4-tetrahydroisoquinolin-6-yl)carbamate (380 mg, 886.76 mol) and 3-bromopiperidine-2,6-dione (119.19 mg, 620.73 mol) in ACN (2.5 mL) was added NaHCO₃ (372.47 mg, 4.43 mmol, 172.52 μL) at 20° C. The mixture was stirred at 80° C. for 16 h. LCMS showed 24% of benzyl (2-(1-(4-aminophenyl)azetidin-3-yl)-1,2,3,4-tetrahydroisoquinolin-6-yl)carbamate was remained and 38% peak with desired mass was detected. The reaction mixture was filtered. The filter cake was washed with mixture solution (40 mL, EtOAc:MeOH:DCM=1:1.5:1.5) and the filtrate was concentrated in vacuum to afford the crude product. The crude product was purified by perp-TLC (SiO₂, DCM:MeOH=10:1) and concentrated in vacuum to afford product A (125 mg), and the purity of the product A was 55% by LCMS. The product A was combined with another pot (72 mg) for re-purified. The mixture was further purified by perp-HPLC (column: Phenomenex luna C18 150*25 mm*10 um; mobile phase: [water(TFA)-ACN]; gradient: 21%-51% B over 9 min) and lyophilized to afford benzyl (2-(1-(4-((2,6-dioxopiperidin-3-yl)amino)phenyl)azetidin-3-yl)-1,2,3,4-tetrahydroisoquinolin-6-yl)carbamate (130 mg, 226.45 μmol, 25.54% yield, 94% purity, TFA) as a purple solid. MS(M+H)⁺=540.3.

### Step 8. Synthesis of 3-((4-(3-(6-amino-3,4-dihydroisoquinolin-2(1H)-yl)azetidin-1-yl)phenyl)amino)piperidine-2,6-dione (9)

A mixture of benzyl (2-(1-(4-((2,6-dioxopiperidin-3-yl)amino)phenyl)azetidin-3-yl)-1,2,3,4-tetrahydroisoquinolin-6-yl)carbamate (110 mg, 168.29 μmol, TFA) in TFA (5.07 g, 44.43 mmol, 3.30 mL) was stirred at 60° C. for 3.5 h. LCMS showed starting material was consumed completely and desired mass (Rₜ=0.110 min) was detected. The reaction mixture was concentrated in vacuum to afford 3-((4-(3-(6-amino-3,4-dihydroisoquinolin-2(1H)-yl)azetidin-1-yl)phenyl)amino)piperidine-2,6-dione (120 mg, crude, TFA) as a black green solid. MS(M+H)⁺=406.3.

### Step 9. Synthesis of N-(2-chlorophenyl)-4-((2-((4-((2-(1-(4-((2,6-dioxopiperidin-3-yl)amino)phenyl)azetidin-3-yl)-1,2,3,4-tetrahydroisoquinolin-6-yl)carbamoyl)phenyl)amino)-5-fluoropyrimidin-4-yl)amino)benzamide (Compound 36)

To a solution of 4-((4-((4-((2-chlorophenyl)carbamoyl)phenyl)amino)-5-fluoropyrimidin-2-yl)amino)benzoic acid (73.59 mg, 153.99 mol) in DMF (0.5 mL) were added HATU (73.19 mg, 192.49 μmol) and DIPEA (74.63 mg, 577.46 μmol) at 20° C. After stirring for 10 min, a solution of 3-((4-(3-(6-amino-3,4-dihydroisoquinolin-2(1H)-yl)azetidin-1-yl)phenyl)amino)piperidine-2,6-dione (100 mg, 192.49 μmol, TFA) in DMF (0.5 mL) with DIPEA (298.53 mg, 2.31 mmol, 402.33 μL) was added at 20° C. The reaction mixture was stirred at 20° C. for 1 h. LCMS showed 1% peak with 4-((4-((4-((2-chlorophenyl)carbamoyl)phenyl)amino)-5-fluoropyrimidin-2-yl)amino) benzoic acid was remained and 73% peak with desired mass (Rₜ=0.802 min) was detected. The reaction mixture was diluted with H₂O (5 mL) and EtOAc (5 ml). The mixture was filtered. The filter cake was washed by EtOAc (8 mL) and concentrated in vacuum to give a residue. The residue was purified by prep-HPLC (column: Phenomenex luna C18 150*25 mm*10 um; mobile phase: [water(TFA)-ACN]; gradient: 26%-56% B over 10 min) and lyophilized to afford N-(2-chlorophenyl)-4-((2-((4-((2-(1-(4-((2,6-dioxopiperidin-3-yl)amino)phenyl)azetidin-3-yl)-1,2,3,4-tetrahydroisoquinolin-6-yl)carbamoyl)phenyl)amino)-5-fluoropyrimidin-4-yl)amino)benzamide (37 mg, 27.16 μmol, 14.11% yield, 97% purity, 4TFA) as a purple solid. MS(M+H)⁺=865.1.

¹H NMR (400 MHz, DMSO-d₆) δ=10.79 (s, 1H), 10.09 (s, 1H), 9.93 (s, 1H), 9.78 (s, 1H), 9.73 (s, 1H), 8.27 (d, J=3.5 Hz, 1H), 8.06-8.00 (m, 4H), 7.95-7.85 (m, 4H), 7.74 (s, 1H), 7.68-7.60 (m, 2H), 7.56 (dd, J=1.3, 7.9 Hz, 1H), 7.39 (dt, J=1.3, 7.7 Hz, 1H), 7.33-7.27 (m, 1H), 7.18 (d, J=8.9 Hz, 1H), 6.70 (br d, J=8.7 Hz, 2H), 6.42 (br d, J=8.4 Hz, 2H), 4.64-4.44 (m, 1H), 4.41-4.26 (m, 2H), 4.21 (br dd, J=4.4, 11.0 Hz, 1H), 4.15-4.05 (m, 2H), 3.98-3.93 (m, 2H), 3.19-2.99 (m, 3H), 2.79-2.68 (m, 1H), 2.58-2.52 (m, 2H), 2.19-1.96 (m, 2H), 1.90-1.79 (m, 1H).

### Example 37. Synthesis of N-(2-chlorophenyl)-4-((2-((4-((1R,5S,6s)-6-((4-(4-((2,6-dioxopiperidin-3-yl) amino)phenyl)piperazin-1-yl)methyl)-3-azabicyclo[3.1.0]hexane-3-carbonyl)phenyl)amino)-5-fluoropyrimidin-4-yl)amino)benzamide (Compound 37)

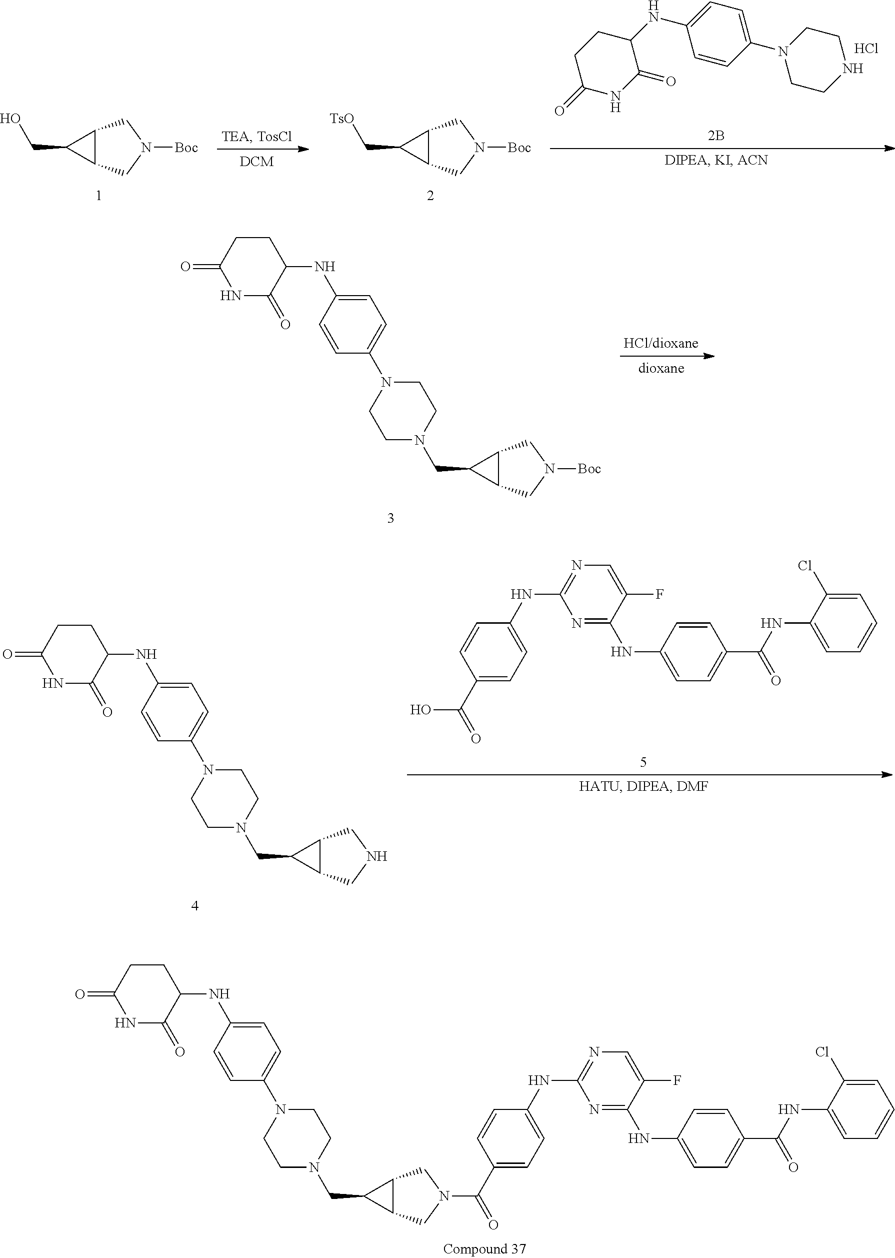

### Step 1. Synthesis of tert-butyl (1R,5S,6r)-6-((tosyloxy)methyl)-3-azabicyclo[3.1.0]hexane-3-carboxylate (2)

To a solution of tert-butyl (1R,5S,6r)-6-(hydroxymethyl)-3-azabicyclo[3.1.0]hexane-3-carboxylate (0.5 g, 2.34 mmol) in DCM (8 mL) were added TosCl (670.43 mg, 3.52 mmol) and TEA (474.46 mg, 4.69 mmol). The mixture was stirred at 25° C. for 12h. TLC (petroleum ether:EtOAc=3:1) showed the starting material was consumed completely and several new spots were formed. The reaction mixture was diluted with water (30 mL) and extracted with DCM (20 mL×3). The combined organic layers were washed with saturated brine (50 mL), dried over anhydrous Na₂SO₄, filtered and concentrated under reduced pressure. The residue was purified by flash silica gel chromatography (ISCO®; 20 g SepaFlash® Silica Flash Column, eluent of 0-30% petroleum ether/EtOAc gradient @80 mL/min) to afford tert-butyl (1R,5S,6r)-6-((tosyloxy)methyl)-3-azabicyclo[3.1.0]hexane-3-carboxylate (250 mg, 391.88 μmol, 16.72% yield, 57.6% purity) as yellow oil. MS(M−t−Bu+H)⁺=312.1.

### Step 2. Synthesis of tert-butyl (1R,5S,6s)-6-((4-(4-((2,6-dioxopiperidin-3-yl)amino)phenyl)piperazin-1-yl)methyl)-3-azabicyclo[3.1.0]hexane-3-carboxylate (3)

To a solution of 3-((4-(piperazin-1-yl)phenyl)amino)piperidine-2,6-dione (260 mg, 701.22 μmol HCl) and tert-butyl (1R,5S,6r)-6-((tosyloxy)methyl)-3-azabicyclo[3.1.0]hexane-3-carboxylate (257.67 mg, 701.22 mol) in ACN (5 mL) was added KI (11.64 mg, 70.12 mol) and DIPEA (181.25 mg, 1.40 mmol). The mixture was stirred at 45° C. for 12 h. LCMS showed 10% of desired compound was detected. The residue was diluted with H₂O (20 mL) and extracted with solvent EtOAc (20 mL×3). The combined organic layers were washed with brine (20 mL), dried over Na₂SO₄, filtered and concentrated under reduced pressure to give a residue. The residue was purified by flash silica gel chromatography (ISCO®; 20 g SepaFlash® Silica Flash Column, eluent of 0~50% EtOAc/petroleum ether, gradient @ 80 mL/min) to afford tert-butyl (1R,5S,6s)-6-((4-(4-((2,6-dioxopiperidin-3-yl)amino)phenyl)piperazin-1-yl)methyl)-3-azabicyclo[3.1.0]hexane-3-carboxylate (45 mg, 82.82 μmol, 11.81% yield, 89% purity) as a white solid. MS(M+H)⁺=484.2.

### Step 3. Synthesis of 3-((4-(4-(((1R,5S,6r)-3-azabicyclo[3.1.0]hexan-6-yl)methyl)piperazin-1-yl)phenyl)amino)piperidine-2,6-dione (4)

To a solution of tert-butyl (1R,5S,6s)-6-((4-(4-((2,6-dioxopiperidin-3-yl)amino)phenyl)piperazin-1-yl)methyl)-3-azabicyclo[3.1.0]hexane-3-carboxylate (45 mg, 93.05 μmol) in dioxane (6 mL) was added HCl/dioxane (4 M, 46.53 μL). The mixture was stirred at 20° C. for 5 hr. LCMS showed the starting material was consumed completely. The reaction mixture was concentrated under reduced pressure to afford 3-((4-(4-(((1R,5S,6r)-3-azabicyclo[3.1.0]hexan-6-yl)methyl)piperazin-1-yl)phenyl)amino)piperidine-2,6-dione (73 mg, crude, HCl) as a purple solid. MS(M+H)⁺=384.3.

### Step 4. Synthesis of N-(2-chlorophenyl)-4-((2-((4-((1R,5S,6s)-6-((4-(4-((2,6-dioxopiperidin-3-yl) amino)phenyl)piperazin-1-yl)methyl)-3-azabicyclo[3.1.0]hexane-3-carbonyl)phenyl)amino)-5-fluoropyrimidin-4-yl)amino)benzamide (Compound 37)

To a solution of 3-((4-(4-(((1R,5S,6r)-3-azabicyclo[3.1.0]hexan-6-yl)methyl)piperazin-1-yl)phenyl)amino)piperidine-2,6-dione (68 mg, 161.93 μmol, HCl) and 4-((4-((4-((2-chlorophenyl) carbamoyl)phenyl)amino)-5-fluoropyrimidin-2-yl)amino)benzoic acid (77.38 mg, 161.93 μmol) in DMF (2 mL) was added HATU (73.88 mg, 194.31 μmol) and DIPEA (62.78 mg, 485.78 μmol, 84.61 μL). The mixture was stirred at 25° C. for 12 hr. LCMS showed 26% peak of desired compound was detected. The residue was diluted with H₂O (3 mL) and extracted with EtOAc (5 mL×3). The combined organic layers were washed with brine (10 mL), dried over Na₂SO₄, filtered and concentrated under reduced pressure to give a residue. The crude product was purified by prep-HPLC (column: Phenomenex luna C18 150*25 mm*10 um; mobile phase: [water (FA)-ACN]; gradient: 12%-42% B over 10 min) and lyophilized to afford N-(2-chlorophenyl)-4-[[2-[4-[(1R,5S)-6-[[4-[4-[(2,6-dioxo-3-piperidyl)amino]phenyl]piperazin-1-yl]methyl]-3-azabicyclo[3.1.0]hexane-3-carbonyl]anilino]-5-fluoro-pyrimidin-4-yl]amino]benzamide (10 mg, 11.25 μmol, 6.95% yield, 94.9% purity) as a purple solid. MS(M+H)⁺=843.4.

¹H NMR (400 MHz, DMSO-d₆) δ=10.77-10.74 (m, 1H), 9.95-9.92 (m, 1H), 9.73 (s, 1H), 9.57 (s, 1H), 8.23 (d, J=3.5 Hz, 1H), 8.17 (s, 1H), 8.10-7.95 (m, 4H), 7.74 (br d, J=8.6 Hz, 2H), 7.65 (d, J=8.4 Hz, 1H), 7.56 (d, J=7.7 Hz, 1H), 7.45-7.38 (m, 3H), 7.32-7.26 (m, 1H), 6.72 (br d, J=8.8 Hz, 2H), 6.59 (br d, J=8.7 Hz, 2H), 5.41-5.32 (m, 1H), 4.21-4.14 (m, 1H), 4.05-3.97 (m, 1H), 3.78-3.68 (m, 2H), 3.51-3.47 (m, 2H), 2.95-2.85 (m, 4H), 2.75-2.65 (m, 1H), 2.62-2.54 (m, 5H), 2.35-2.25 (m, 2H), 2.15-2.05 (m, 1H), 1.48-1.40 (m, 2H).

### Example 38. Synthesis of N-(2-chlorophenyl)-4-[[2-[4-[[4-[[[1-[4-[(2,6-dioxo-3-piperidyl)amino]phenyl]-4-piperidyl]methyl-methyl-amino]methyl]cyclohexyl]carbamoyl]anilino]-5-fluoro-pyrimidin-4-yl]amino]benzamide (Compound 38)

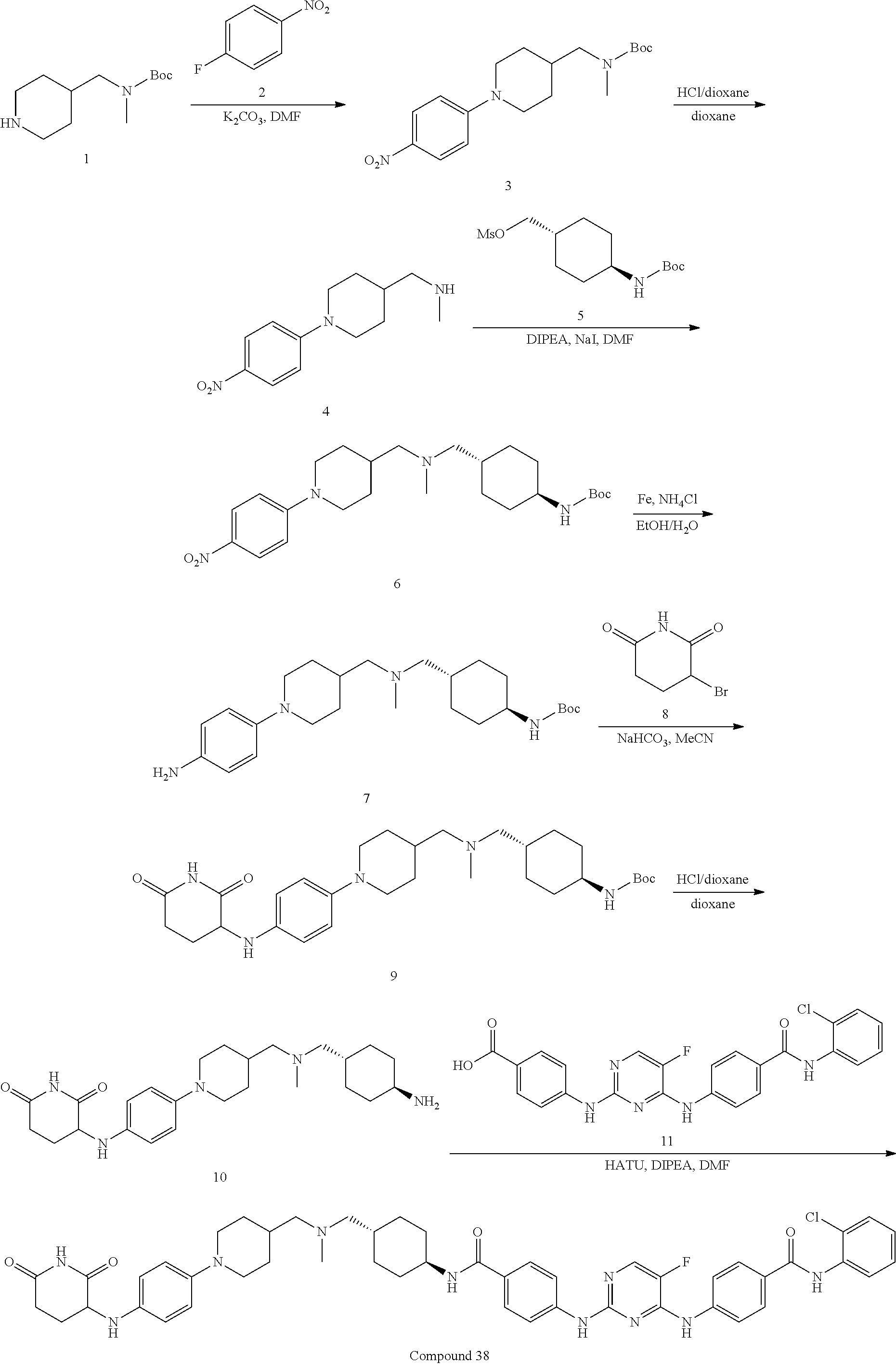

### Step 1. Synthesis of tert-butyl N-methyl-N-[[1-(4-nitrophenyl)-4-piperidyl]methyl]carbamate (3)

To a solution of tert-butyl N-methyl-N-(4-piperidylmethyl)carbamate (1.5 g, 6.57 mmol) and 1-fluoro-4-nitro-benzene (926.95 mg, 6.57 mmol) in DMF (15 mL) was added K₂CO₃ (1.82 g, 13.14 mmol) at 20° C. The mixture was stirred at 80° C. for 16 hr. LCMS showed a peak (84%) with the mass of desired product. The reaction mixture was diluted with water (50 mL) and extracted with EtOAc (30 mL×3). The combined organic phase was washed with brine (20 ml×3), dried over Na₂SO₄ and concentrated under reduced pressure to afford a residue. The residue was purified by flash silica gel chromatography (20 g SepaFlash® Silica Flash Column, eluent of 0-30% EtOAc/petroleum ether, gradient @ 80 mL/min) to afford tert-butyl N-methyl-N-[[1-(4-nitrophenyl)-4-piperidyl]methyl]carbamate (2.15 g, 6.03 mmol, 91.79% yield, 98% purity) as light yellow solid. MS(M+H)⁺=350.2.

### Step 2. Synthesis of N-methyl-1-[1-(4-nitrophenyl)-4-piperidyl]methanamine (4)

To a solution of tert-butyl N-methyl-N-[[1-(4-nitrophenyl)-4-piperidyl]methyl]carbamate (1 g, 2.86 mmol) in dioxane (4 mL) was added HCl/dioxane (4 M, 4 mL). The mixture was stirred at 20° C. for 1 hr. LCMS showed a peak (97%) with the mass of desired product. The reaction mixture was concentrated under reduced pressure to afford a residue. The residue was triturated with 20 ml of EtOAc to afford N-methyl-1-[1-(4-nitrophenyl)-4-piperidyl]methanamine (700 mg, 2.81 mmol, 98.11% yield, 100% purity) as light yellow solid. MS(M+H)⁺=250.2.

### Step 3. Synthesis of tert-butyl N-[4-[[methyl-[[1r-(4-nitrophenyl)-4-piperidyl]methyl]amino]methyl]cyclohexyl]carbamate (6)

To a solution of N-methyl-1-[1-(4-nitrophenyl)-4-piperidyl]methanamine (1 g, 3.50 mmol, HCl) and [4-(tert-butoxycarbonylamino)cyclohexyl]methyl methanesulfonate (2.15 g, 7.00 mmol) in DMF (15 mL) were added DIPEA (1.81 g, 14.00 mmol, 2.44 mL) and NaI (104.90 mg, 699.87 μmol) at 20° C. The mixture was stirred at 80° C. for 16 hr. LCMS showed a peak (26%) with the mass of desired product and starting material remained. To the mixture was added [4-(tert-butoxycarbonylamino)cyclohexyl]methyl methanesulfonate (1.08 g, 3.50 mmol) and the resulting mixture was stirred at 80° C. for 24 hr. LCMS showed a peak (53%, 254 nm) with the mass of desired product. The reaction mixture was diluted with water (60 mL) and extracted with EtOAc (20 mL×3). The combined organic layers were washed with brine (20 mL×3), dried over Na₂SO₄, filtered and concentrated under reduced pressure. The residue was purified by flash silica gel chromatography (12 g SepaFlash® Silica Flash Column, eluent of 0~20% MeOH/EtOAc, gradient @60 mL/min) to afford tert-butyl N-[4-[[methyl-[[1-(4-nitrophenyl)-4-piperidyl]methyl]amino]methyl]cyclohexyl]carbamate (1.4 g, 3.04 mmol, 86.86% yield, 100% purity) as yellow oil. MS(M+H)⁺=461.3.

### Step 4. Synthesis of tert-butyl N-[4-[[[1-(4-aminophenyl)-4-piperidyl]methyl-methyl-amino]methyl]cyclohexyl]carbamate (7)

To a solution of tert-butyl N-[[methyl-[[1-(4-nitrophenyl)-4-piperidyl]methyl]amino]methyl]cyclohexyl]carbamate (1.4 g, 3.04 mmol) in EtOH (10 mL) and H₂O (10 mL) were added Fe (509.22 mg, 9.12 mmol) and NH₄Cl (812.92 mg, 15.20 mmol). The mixture was stirred at 80° C. for 16 hr. LCMS showed a peak (82%) with the mass of desired product. The reaction mixture was filtrated through celite pad and the filtrate was concentrated under reduced pressure. The residue was diluted with aqueous Na₂CO₃ solution (20 mL) and extracted with DCM (10 ml×3). The combined organic phase was concentrated under reduced pressure. The residue was triturated with 15 ml of MeCN and 2 ml of EtOAc to afford tert-butyl N-[4-[[[1-(4-aminophenyl)-4-piperidyl]methyl-methyl-amino]methyl]cyclohexyl]carbamate (200 mg, 464.44 μmol, 15.28% yield, 100% purity) as brown solid. MS(M+H)⁺=431.3.

### Step 5. Synthesis of tert-butyl ((1r,4r)-4-((((1-(4-((2,6-dioxopiperidin-3-yl)amino)phenyl)piperidin-4-yl)methyl)(methyl)amino)methyl)cyclohexyl)carbamate (9)

To a solution of tert-butyl N-[4-[[[1-(4-aminophenyl)-4-piperidyl]methyl-methyl-amino]methyl]cyclohexyl]carbamate (200 mg, 464.44 μmol) and 3-bromopiperidine-2,6-dione (222.94 mg, 1.16 mmol) in MeCN (1 mL) was added NaHCO₃ (156.06 mg, 1.86 mmol). The mixture was stirred at 80° C. for 16 hr. LCMS showed a peak (19%) with the mass of desired product. The reaction mixture was diluted with water (10 mL) and filtered. The filter cake was collected and dried to afford tert-butyl N-[4-[[[1-[4-[(2,6-dioxo-3-piperidyl)amino]phenyl]-4-piperidyl]methyl-methyl-amino]methyl]cyclohexyl]carbamate (150 mg, 276.89 μmol, 59.62% yield, 100% purity) as black solid. MS(M+H)⁺=542.4.

### Step 6. Synthesis of 3-[4-[4-[[(4-aminocyclohexyl)methyl-methyl-amino]methyl]-1-piperidyl]anilino]piperidine-2,6-dione (10)

To a solution of tert-butyl N-[4-[[[1-[4-[(2,6-dioxo-3-piperidyl)amino]phenyl]-4-piperidyl]methyl-methyl-amino]m ethyl]cyclohexyl]carbamate (150 mg, 276.89 mol) in dioxane (1 mL) was added HCl/dioxane (4 M, 1 mL). The mixture was stirred at 15° C. for 1 hr. LCMS showed a peak (80%) with the mass of desired product. The reaction mixture was concentrated under reduced pressure to afford 3-[4-[4-[[(4-aminocyclohexyl)methyl-methyl-amino]methyl]-1-piperidyl]anilino]piperidine-2,6-dione (130 mg, 271.93 μmol, 98.21% yield, HCl) as blue solid. MS(M+H)⁺=442.3.

### Step 7. Synthesis of N-(2-chlorophenyl)-4-[[2-[4-[[4-[[[1-[4-[(2,6-dioxo-3-piperidyl)amino]phenyl]-4-piperidyl]methyl-methyl-amino]methyl]cyclohexyl]carbamoyl]anilino]-5-fluoro-pyrimidin-4-yl]amino]benzamide (Compound 38)

To a solution of 3-[4-[4-[[(4-aminocyclohexyl)methyl-methyl-amino]methyl]-1-piperidyl]anilino]piperidine-2,6-dione (110 mg, 230.09 μmol, HCl) and 4-[[4-[4-[(2-chlorophenyl) carbamoyl]anilino]-5-fluoro-pyrimidin-2-yl]amino]benzoic acid (109.96 mg, 230.09 mol) in DMF (2 mL) was added HATU (131.23 mg, 345.14 μmol) and DIPEA (89.21 mg, 690.28 μmol). The mixture was stirred at 15° C. for 16 hr. LCMS showed a peak (48%) with the mass of desired product. The reaction mixture was diluted with EtOAc (20 mL) and filtered. The filter cake was purified by prep-HPLC(column: Phenomenex luna C18 150*25 mm*10 um; mobile phase: [water(TFA)-ACN]; gradient: 20%-50% B over 10 min) and further purified by prep-HPLC(column: Waters Xbridge 150*25 mm*5 um; mobile phase: [water(NH₄HCO₃)-ACN]; gradient: 45%-75% B over 10 min). The fractions was lyophilized to afford N-(2-chlorophenyl)-4-[[2-[4-[[4-[[[1-[4-[(2,6-dioxo-3-piperidyl)amino]phenyl]-4-piperid yl]methyl-methyl-amino]methyl]cyclohexyl]carbamoyl]anilino]-5-fluoro-pyrimidin-4-yl]amino]benzamide (6.6 mg, 7.17 μmol, 3.12% yield, 98% purity) as a white solid. MS(M+H)⁺=901.5.

¹H NMR (400 MHz, DMSO-d₆) δ=10.77 (s, 1H), 9.94 (s, 1H), 9.75 (s, 1H), 9.61 (s, 1H), 8.25 (d, J=3.4 Hz, 1H), 8.07-7.93 (m, 5H), 7.80-7.75 (m, 4H), 7.70-7.62 (m, 1H), 7.57 (dd, J=1.2, 7.8 Hz, 1H), 7.43-7.35 (m, 1H), 7.34-7.25 (m, 1H), 6.75 (d, J=8.9 Hz, 2H), 6.60 (d, J=8.7 Hz, 2H), 5.36 (d, J=7.2 Hz, 1H), 4.25-4.14 (m, 1H), 3.80-3.62 (m, 1H), 3.38-3.36 (m, 1H), 2.17-2.05 (m, 9H), 1.92-1.71 (m, 9H), 1.59-1.45 (m, 2H), 1.34-1.28 (m, 2H), 1.20-1.05 (m, 4H), 0.95-0.88 (m, 2H).

### Example 39. Synthesis of N-(2-chlorophenyl)-4-((2-((4-(((1r,4r)-4-((4-(4-((2,6-dioxopiperidin-3-yl)amino)phenyl)-1,4-diazepan-1-yl)methyl)cyclohexyl)carbamoyl)phenyl)amino)-5-fluoropyrimidin-4-yl)amino)benzamide (Compound 39)

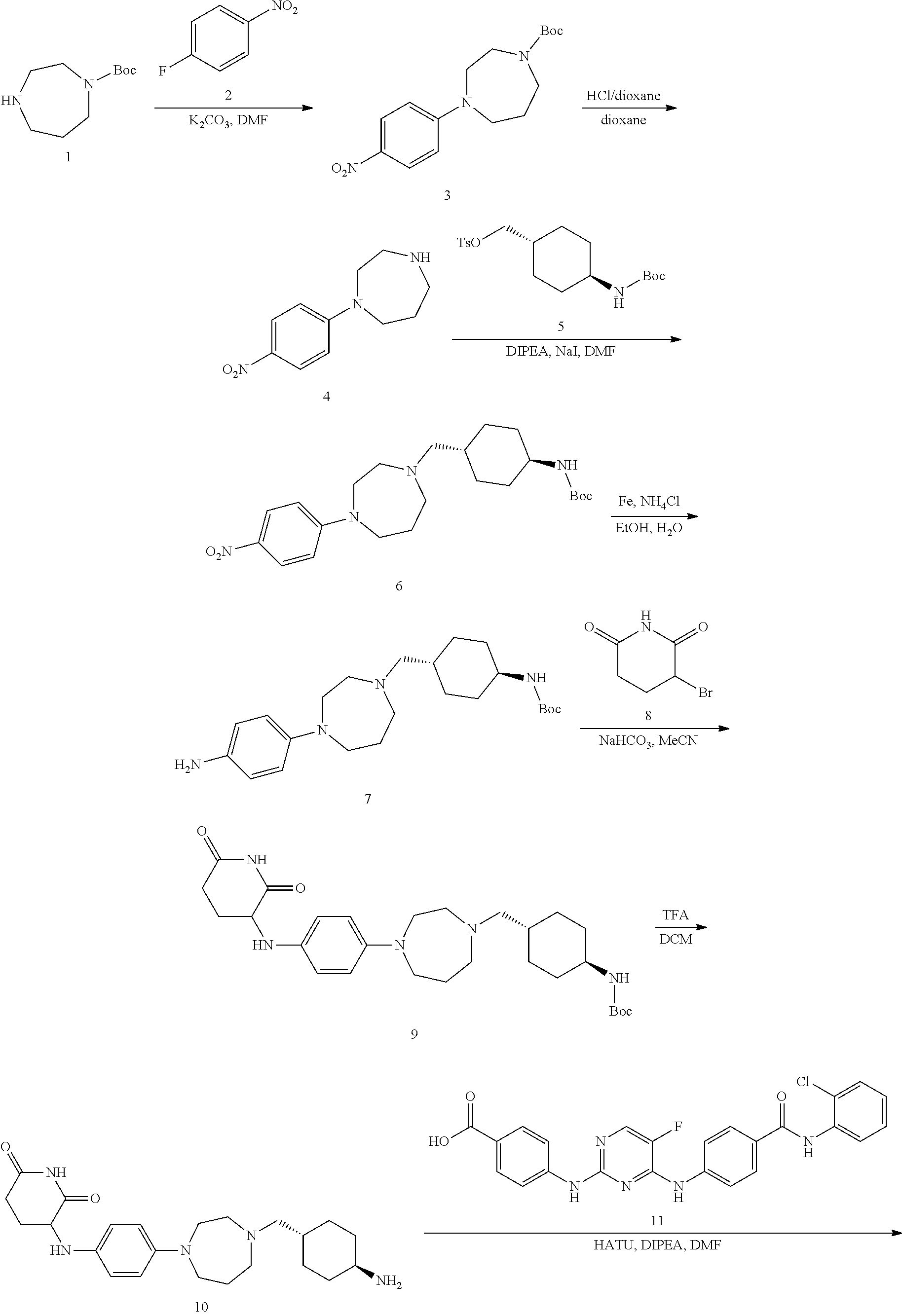

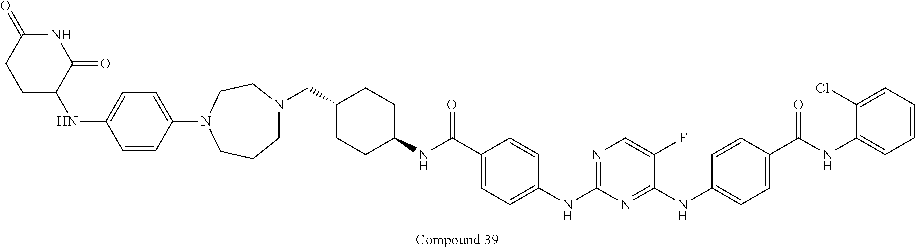 [SMILES: O=C1CCC(Nc2ccc(N3CCCN(C[C@H]4CC[C@H](NC(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of tert-butyl 4-(4-nitrophenyl)-1,4-diazepane-1-carboxylate (3)

To a solution of tert-butyl 1,4-diazepane-1-carboxylate (5 g, 24.97 mmol, 4.92 mL) and 1-fluoro-4-nitrobenzene (4.23 g, 29.96 mmol, 3.18 mL) in DMF (50 mL) was added K₂CO₃ (5.18 g, 37.45 mmol) at 20° C. The mixture was stirred at 20° C. for 16 h. LCMS showed the desired mass was detected. The reaction mixture was diluted with water (100 mL) and extracted with EtOAc (60 mL×3). The combined organic layers was washed with brine (30 mL×3), dried over Na₂SO₄, filtered and concentrated in vacuum and then purified by flash silica gel chromatography (40 g SepaFlash® Silica Flash Column, eluent of 0-35% EtOAc/petroleum ether, gradient @ 100 mL/min) to afford tert-butyl 4-(4-nitrophenyl)-1,4-diazepane-1-carboxylate (6.2 g, 19.10 mmol, 76.50% yield, 99% purity) as a yellow solid. MS(M−t−Bu+H)⁺=266.4.

### Step 2. Synthesis of 1-(4-nitrophenyl)-1,4-diazepane (4)

To a solution of tert-butyl 4-(4-nitrophenyl)-1,4-diazepane-1-carboxylate (3 g, 9.34 mmol) in dioxane (4 mL) was added HCl/dioxane (4 M, 30 mL) at 20° C. The mixture was stirred at 20° C. for 1 hr. LCMS showed the starting material was consumed completely and desired mass was detected. The reaction mixture was concentrated in vacuum to afford 1-(4-nitrophenyl)-1,4-diazepane (3 g, crude, HCl) as a yellow solid. MS(M+H)⁺=222.1.

### Step 3. Synthesis of tert-butyl

### ((1r,4r)-4-((4-(4-nitrophenyl)-1,4-diazepan-1-yl)methyl)cyclohexyl) carbamate (6)

To a solution of 1-(4-nitrophenyl)-1,4-diazepane (0.5 g, 1.94 mmol, HCl) in DMF (6 mL) were added DIPEA (752.24 mg, 5.82 mmol, 1.01 mL), NaI (29.08 mg, 194.01 μmol) and ((1r,4r)-4-((tert-butoxycarbonyl)amino)cyclohexyl)methyl 4-methylbenzenesulfonate (1.12 g, 2.91 mmol). The resulting mixture was stirred at 60° C. for 14 h. LCMS showed 36% peak of 1-(4-nitrophenyl)-1,4-diazepane and 43% peak of the desired mass. Another portion of ((1r,4r)-4-((tert-butoxycarbonyl)amino)cyclohexyl)methyl 4-methylbenzenesulfonate (744 mg, 1.94 mmol) was added at 20° C. and the mixture was stirred at 60° C. for 14 h. LCMS showed 58% of the desired mass. The mixture was diluted with water (15 mL) and extracted with EtOAc (10 mL). The combined organic layer was washed with brine (10 mL), dried over Na₂SO₄ and concentrated under reduced pressure. The residue was purified by flash silica gel chromatography (5 g SepaFlash® Silica Flash Column, eluent of 40-100% EtOAc/petroleum ether, gradient @ 50 mL/min) and then the product was diluted with MTBE (10 mL) and stirred at 20° C. for 0.5 h. The mixture was filtered and the filter cake was washed with MTBE (20 mL), collected and dried to afford tert-butyl ((1r,4r)-4-((4-(4-nitrophenyl)-1,4-diazepan-1-yl)methyl)cyclohexyl)carbamate (0.5 g, 1.09 mmol, 56.01% yield, 94% purity) as a yellow solid. MS(M+H)⁺=433.4.

SFC Method Com: Column: Chiralcel OJ-3 50×4.6 mm I. D., 3 um; Mobile phase: Phase A for CO₂, and Phase B for EtOH (0.05% DEA); Gradient elution: B in A from 5% to 40%; Flow rate: 3 mL/min; Detector: DAD; Column Temp: 35 C; Back Pressure: 100 Bar.

### Step 4. Synthesis of tert-butyl ((1r,4r)-4-((4-(4-aminophenyl)-1,4-diazepan-1-yl)methyl)cyclohexyl) carbamate (7)

To a mixture of tert-butyl ((1r,4r)-4-((4-(4-nitrophenyl)-1,4-diazepan-1-yl)methyl)cyclohexyl) carbamate (0.4 g, 924.74 mol) and NH₄Cl (248 mg, 4.64 mmol) in EtOH (12 mL) and H₂O (4 mL) was added Fe (258 mg, 4.62 mmol) and the mixture was stirred at 80° C. for 1 h. LCMS showed 82% peak of the desired mass. The mixture was combined with other batch (0.1 g scale) and then filtered and washed with EtOH (20 mL) and THF (30 mL). The filtrate was concentrated under reduced pressure. The crude was diluted with EtOAc (10 mL) and water (10 mL) and then the mixture was adjusted the pH=9 with saturation Na₂CO₃. The mixture was extracted with EtOAc (10 mL×3), the combined organic layers was washed with water (10 mL), dried over Na₂SO₄, filtered and concentrated under reduced pressure to afford tert-butyl ((1r,4r)-4-((4-(4-aminophenyl)-1,4-diazepan-1-yl)methyl)cyclohexyl)carbamate (0.5 g, crude) as a yellow solid. MS(M+H)⁺=403.4.

### Step 5. Synthesis of tert-butyl ((1r,4r)-4-((4-(4-((2,6-dioxopiperidin-3-yl)amino)phenyl)-1,4-diazepan-1-yl)methyl)cyclohexyl)carbamate (9)

A solution of tert-butyl ((1r,4r)-4-((4-(4-aminophenyl)-1,4-diazepan-1-yl)methyl)cyclohexyl) carbamate (450 mg, 1.12 mmol), 3-bromopiperidine-2,6-dione (324 mg, 1.69 mmol) and NaHCO₃ (282 mg, 3.36 mmol, 130.62 μL) in MeCN (4.5 mL) was stirred at 80° C. for 14 h. LCMS showed 95% peak of the desired mass. The mixture was filtered and the filter cake was washed with EtOAc/MeOH=10/1 (100 mL). The filtrate was concentrated under reduced pressure. The residue was purified by flash silica gel chromatography (5 g SepaFlash® Silica Flash Column, eluent of 70-100% EtOAc/petroleum ether to 10~20% MeOH/EtOAc, gradient @ 50 mL/min) to afford tert-butyl ((1r,4r)-4-((4-(4-((2,6-dioxopiperidin-3-yl)amino)phenyl)-1,4-diazepan-1-yl)methyl)cyclohexyl)carbamate (290 mg, 525.04 μmol, 46.97% yield, 93% purity) as a blue solid. MS(M+H)⁺=514.5.

### Step 6. Synthesis of 3-((4-(4-(((1r,4r)-4-aminocyclohexyl)methyl)-1,4-diazepan-1-yl)phenyl)amino)piperidine-2,6-dione (10)

To a solution of tert-butyl ((1r,4r)-4-((4-(4-((2,6-dioxopiperidin-3-yl)amino)phenyl)-1,4-diazepan-1-yl)methyl)cyclohexyl)carbamate (150 mg, 292.02 mol) in DCM (1.5 mL) was added TFA (690.75 mg, 6.06 mmol, 450.00 μL) and the mixture was stirred at 20° C. for 1 h. LCMS showed 85% pea of the desired mass. The mixture was concentrated under reduced pressure to afford 3-((4-(4-(((1r,4r)-4-aminocyclohexyl)methyl)-1,4-diazepan-1-yl)phenyl)amino)piperidine-2,6-dione (160 mg, crude, TFA) as a black brown oil. MS(M+H)⁺=414.3.

### Step 7. Synthesis of N-(2-chlorophenyl)-4-((2-((4-(((1r,4r)-4-((4-(4-((2,6-dioxopiperidin-3-yl)amino)phenyl)-1,4-diazepan-1-yl)methyl)cyclohexyl)carbamoyl)phenyl)amino)-5-fluoropyrimidin-4-yl)amino)benzamide (Compound 39)

To a solution of 4-((4-((4-((2-chlorophenyl)carbamoyl)phenyl)amino)-5-fluoropyrimidin-2-yl) amino)benzoic acid (145 mg, 303.43 mol) and HATU (138 mg, 362.94 μmol) in DMF (1.5 mL) was added DIPEA (385.84 mg, 2.99 mmol) and the mixture was stirred at 20° C. for 15 min. Then a solution of 3-((4-(4-(((1r,4r)-4-aminocyclohexyl)methyl)-1,4-diazepan-1-yl)phenyl)amino)piperidine-2,6-dione (160 mg, 303.27 μmol, TFA) in DMF (1.5 mL) at 0° C. was added and the resulting mixture was stirred at 20° C. for 1 h. LCMS showed 86% peak of the desired mass. The mixture was diluted with water (10 mL) and then filtered. The filter cake was washed with water (10 mL), collected and dried. The crude product was purified by prep-TLC (DCM:MeOH=7:1) and then re-purified by prep-HPLC (column: Phenomenex luna C18 150*25 mm*10 um; mobile phase: [water (TFA)-ACN]; gradient: 20%-50% B over 10 min) and lyophilized. The product was further purified by prep-HPLC (column: Phenomenex luna C18 150*25 mm*10 um; mobile phase: [water (TFA)-ACN]; gradient: 25%-55% B over 10 min) and lyophilized to afford N-(2-chlorophenyl)-4-((2-((4-(((1r,4r)-4-((4-(4-((2,6-dioxopiperidin-3-yl)amino)phenyl)-1,4-diazepan-1-yl)methyl)cyclohexyl)carbamoyl)phenyl)amino)-5-fluoropyrimidin-4-yl)amino)benzamide (42.6 mg, 31.44 μmol, 10.37% yield, 89.7% purity, 3TFA) as a red solid. MS(M+H)⁺=873.4.

¹H NMR (400 MHz, DMSO-d₆) δ=10.85 (br s, 1H), 9.94 (s, 1H), 9.76 (s, 1H), 9.62 (s, 1H), 9.01-8.89 (m, 1H), 8.25 (d, J=3.5 Hz, 1H), 8.07-7.97 (m, 5H), 7.81-7.72 (m, 4H), 7.67-7.63 (m, 1H), 7.59-7.55 (m, 1H), 7.43-7.37 (m, 1H), 7.33-7.26 (m, 1H), 6.75-6.65 (m, 3H), 4.28-4.20 (m, 1H), 3.76-3.64 (m, 2H), 3.62-3.47 (m, 2H), 3.41-3.27 (m, 1H), 3.24-3.11 (m, 1H), 3.08-3.00 (m, 1H), 2.80-2.68 (m, 1H), 2.44-2.36 (m, 5H), 2.21-2.13 (m, 2H), 2.09-2.01 (m, 1H), 1.94-1.77 (m, 6H), 1.74-1.65 (m, 1H), 1.42-1.28 (m, 2H), 1.16-1.04 (m, 2H).

### Example 40. Synthesis of N-(2-chlorophenyl)-4-((2-((4-(((1R,5S,6s)-3-((1-(4-((2,6-dioxopiperidin-3-yl) amino)phenyl)piperidin-4-yl)methyl)-3-azabicyclo[3.1.0]hexan-6-yl)carbamoyl)phenyl) amino)-5-fluoropyrimidin-4-yl)amino)benzamide (Compound 40)

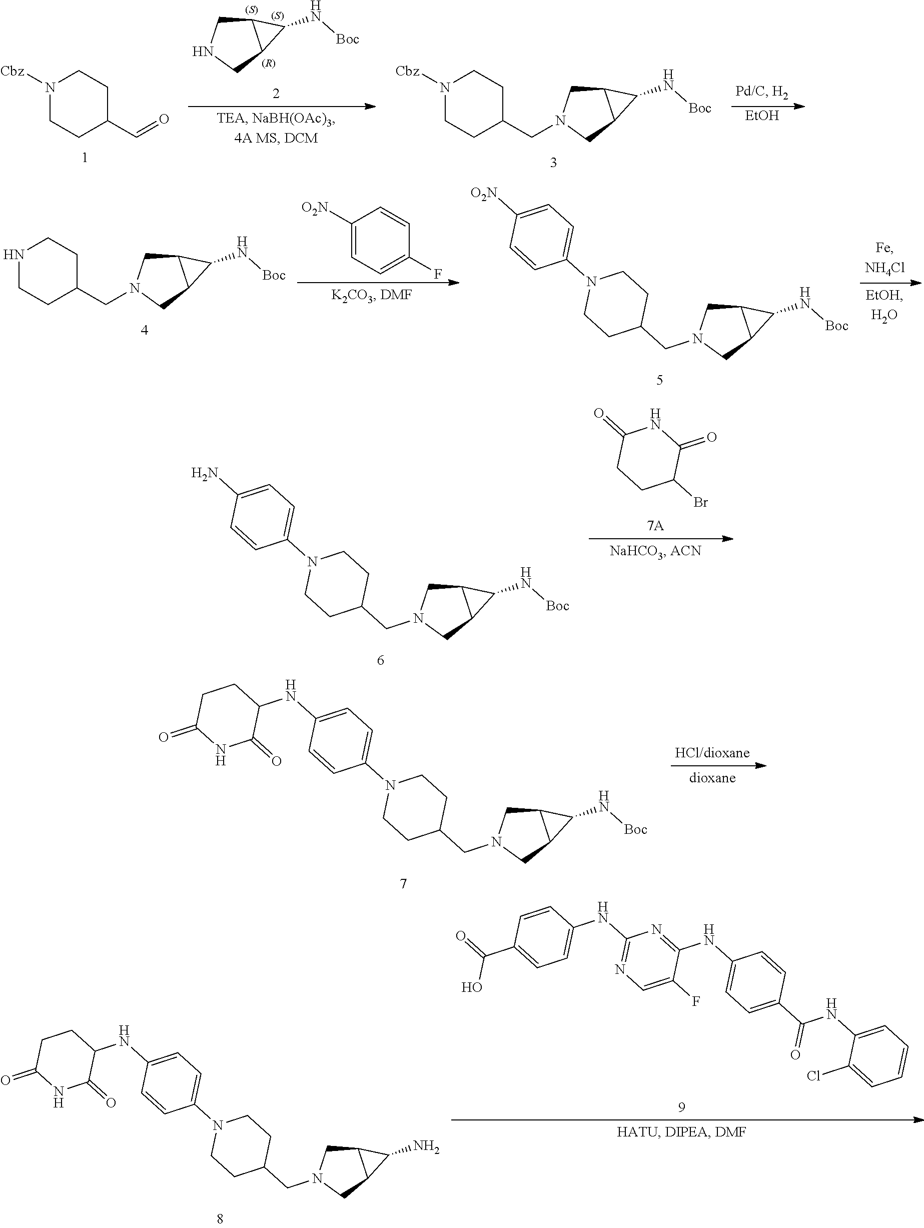

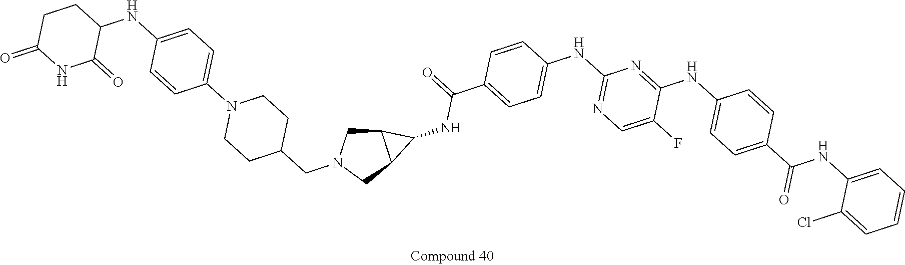 [SMILES: O=C1CCC(Nc2ccc(N3CCC(CN4C[C@@H]5[C@H](C4)[C@H]5NC(=O)c4ccc(Nc5ncc(F)c(Nc6ccc(C(=O)Nc7ccccc7Cl)cc6)n5)cc4)CC3)cc2)C(=O)N1]

### Step 1. Synthesis of benzyl 4-(((1R,5S,6s)-6-((tert-butoxycarbonyl)amino)-3-azabicyclo[3.1.0] hexan-3-yl)methyl)piperidine-1-carboxylate (3)

To a solution of benzyl 4-formylpiperidine-1-carboxylate (1.25 g, 5.05 mmol) in DCM (20 mL) were added TEA (1.53 g, 15.16 mmol, 2.11 mL) and 4 Å MS (50 mg), tert-butyl ((1R,5S,6s)-3-azabicyclo[3.1.0]hexan-6-yl)carbamate (1.00 g, 5.05 mmol) at 20° C. After stirring 10 min, then NaBH(OAc)₃ (3.21 g, 15.16 mmol) was slowly added at 20° C. The reaction mixture was stirred at 20° C. for 16 h. LCMS showed the starting material was consumed completely and 58% peak with desired mass (Rt=0.635 min) was detected. The reaction mixture was filtered. The filter cake was washed by mix solution (50 ml, EtOAc:MeOH=1:2). The filtrate was concentrated in vacuum to afford crude product. The crude product was triturated with EtOAc (10 mL) at 20° C. for 1 h and filtered. The filter cake was washed by EtOAc (30 ml). The filtrate was concentrated in vacuum to afford the crude product, which was purified by flash silica gel chromatography (ISCO®; 20 g SepaFlash® Silica Flash Column, eluent of 30-70% EtOAc/petroleum ether, gradient @ 100 mL/min) to afford benzyl 4-(((1R,5S,6s)-6-((tert-butoxycarbonyl)amino)-3-azabicyclo[3.1.0]hexan-3-yl)methyl) piperidine-1-carboxylate (1.76 g, 4.10 mmol, 81.06% yield) as a colorless oil. MS(M+H)⁺=430.4.

### Step 2. Synthesis of tert-butyl ((1R,5S,6s)-3-(piperidin-4-ylmethyl)-3-azabicyclo[3.1.0]hexan-6-yl)carbamate (4)

A mixture of benzyl 4-(((1R,5S,6s)-6-((tert-butoxycarbonyl)amino)-3-azabicyclo[3.1.0]hexan-3-yl)methyl) piperidine-1-carboxylate (1.76 g, 4.10 mmol) and Pd/C (0.8 g, 10% purity) in EtOH (20 mL) was degassed and purged with H₂ for 3 times. The resulting mixture was stirred under H₂ (15 Psi) atmosphere at 20° C. for 12 h. LCMS showed the starting material was consumed completely and desired mass (Rt=0.797 min) was detected. The reaction mixture was filtered and washed by MeOH (30 ml×3). The filtrate was concentrated in vacuum to afford tert-butyl ((1R,5S,6s)-3-(piperidin-4-ylmethyl)-3-azabicyclo[3.1.0]hexan-6-yl)carbamate (1.2 g, crude) as a gray solid. MS(M+H)⁺=296.2.

### Step 3. Synthesis of tert-butyl ((1R,5S,6s)-3-((1-(4-nitrophenyl)piperidin-4-yl)methyl)-3-azabicyclo[3.1.0]hexan-6-yl)carbamate (5)

To a solution of tert-butyl ((1R,5S,6s)-3-(piperidin-4-ylmethyl)-3-azabicyclo[3.1.0]hexan-6-yl)carbamate (1.2 g, 4.06 mmol) and 1-fluoro-4-nitrobenzene (1.09 g, 7.72 mmol) in DMF (20 mL) was added K₂CO₃ (1.68 g, 12.19 mmol) at 20° C. The mixture was stirred at 20° C. for 12 h. LCMS showed tert-butyl ((1R,5S,6s)-3-(piperidin-4-ylmethyl)-3-azabicyclo[3.1.0]hexan-6-yl)carbamate was remained and desired mass (Rt=0.674 min) was detected. Then heat up reaction mixture to 40° C. and continue stirred for 4 h. LCMS showed tert-butyl ((1R,5S,6s)-3-(piperidin-4-ylmethyl)-3-azabicyclo[3.1.0]hexan-6-yl)carbamate was remained and desired mass was detected. The reaction mixture was continue stirred at 40° C. for 12 h. LCMS showed tert-butyl ((1R,5S,6s)-3-(piperidin-4-ylmethyl)-3-azabicyclo[3.1.0]hexan-6-yl)carbamate was consumed completely and desired mass was detected. The reaction mixture was diluted with H₂O (60 mL) and extracted with EtOAc (30 mL×3). The combined organic layers were washed with brine (30 mL), dried over Na₂SO₄ and concentrated in vacuum to give a residue. The residue was purified by flash silica gel chromatography (ISCO®; 20 g SepaFlash® Silica Flash Column, Eluent of 0-100% EtOAc/Petroleum ether gradient @ 100 mL/min) to afford tert-butyl ((1R,5S,6s)-3-((1-(4-nitrophenyl)piperidin-4-yl)methyl)-3-azabicyclo[3.1.0]hexan-6-yl)carbamate (1.2 g, 2.79 mmol, 68.80% yield, 97% purity) as a yellow solid. MS(M+H)⁺=417.3.

### Step 4. Synthesis of tert-butyl ((1R,5S,6s)-3-((1-(4-aminophenyl)piperidin-4-yl)methyl)-3-azabicyclo[3.1.0]hexan-6-yl)carbamate (6)

To a solution of tert-butyl ((1R,5S,6s)-3-((1-(4-nitrophenyl)piperidin-4-yl)methyl)-3-azabicyclo[3.1.0]hexan-6-yl)carbamate (1.2 g, 2.88 mmol) in EtOH (10 mL) and H₂O (10 mL) were added Fe (643.57 mg, 11.52 mmol) and NH₄Cl (770.56 mg, 14.41 mmol) at 20° C. The mixture was stirred at 80° C. for 2 h. LCMS showed the starting material was consumed completely and desired mass was detected. The reaction mixture was filtered and concentrated in vacuum to give a residue. The residue was diluted with H₂O (50 mL) and adjusted to pH=10 with Na₂CO₃, extracted with EtOAc (20 mL×3). The combined organic layers were dried over Na₂SO₄ and concentrated in vacuum to afford tert-butyl ((1R,5S,6s)-3-((1-(4-aminophenyl)piperidin-4-yl)methyl)-3-azabicyclo[3.1.0]hexan-6-yl)carbamate (970 mg, 2.41 mmol, 83.62% yield, 96% purity) as a purple solid. MS(M+H)⁺=387.4.

### Step 5. Synthesis of tert-butyl ((1R,5S,6s)-3-((1-(4-((2,6-dioxopiperidin-3-yl)amino)phenyl)piperidin-4-yl)methyl)-3-azabicyclo[3.1.0]hexan-6-yl)carbamate (7)

To a solution of tert-butyl ((1R,5S,6s)-3-((1-(4-aminophenyl)piperidin-4-yl)methyl)-3-azabicyclo[3.1.0]hexan-6-yl)carbamate (500 mg, 1.29 mmol) and 3-bromopiperidine-2,6-dione (347.73 mg, 1.81 mmol) in ACN (4 mL) was added NaHCO₃ (543.34 mg, 6.47 mmol) at 20° C. The mixture was stirred at 80° C. for 16 h. LCMS showed 86% peak with the desired mass was detected. The reaction mixture was washed by mixture solution (100 ml, MeOH:EtOAc:THF=1:1:3) and concentrated in vacuum to give a residue. The residue was purified by flash silica gel chromatography (ISCO®; 12 g SepaFlash® Silica Flash Column, eluent of 50-100% EtOAc/petroleum ether to 0~20% DCM:MeOH, gradient @ 100 mL/min) to afford the product, which was further purified by prep-HPLC (column: Phenomenex luna C18 150*40 mm*15 um; mobile phase: [water(TFA)-ACN]; gradient: 1%-31% B over 10 min) and lyophilized to afford tert-butyl ((1R,5S,6s)-3-((1-(4-((2,6-dioxopiperidin-3-yl)amino)phenyl)piperidin-4-yl)methyl)-3-azabicyclo[3.1.0]hexan-6-yl)carbamate (300 mg, 480.67 μmol, 37.16% yield, 98% purity, TFA) as a purple solid. MS(M+H)⁺=498.3.

### Step 6. Synthesis of 3-((4-(4-(((1R,5S,6s)-6-amino-3-azabicyclo[3.1.0]hexan-3-yl)methyl)piperidin-1-yl)phenyl)aminol)piperidine-2,6-dione (8)

To a solution of tert-butyl ((1R,5S,6s)-3-((1-(4-((2,6-dioxopiperidin-3-yl)amino)phenyl)piperidin-4-yl)methyl)-3-azabicyclo[3.1.0]hexan-6-yl)carbamate (150 mg, 301.43 μmol) in dioxane (2 mL) was added HCl/dioxane (4 M, 6 mL) at 20° C. The mixture was stirred at 20° C. for 1 h. LCMS showed the starting material was consumed completely and desired mass was desired. The reaction mixture was concentrated in vacuum to afford 3-((4-(4-(((1R,5S,6s)-6-amino-3-azabicyclo[3.1.0]hexan-3-yl)methyl)piperidin-1-yl)phenyl)amino)piperidine-2,6-dione (180 mg, crude, HCl) as a yellow solid. MS(M+H)⁺=398.3.

### Step 7. Synthesis of N-(2-chlorophenyl)-4-((2-((4-(((1R,5S,6s)-3-((1-(4-((2,6-dioxopiperidin-3-yl)amino)phen yl)piperidin-4-yl)methyl)-3-azabicyclo[3.1.0]hexan-6-yl)carbamoyl)phenyl) amino)-5-fluoropyrimidin-4-yl)amino)benzamide (Compound 40)

To a solution of 4-((4-((4-((2-chlorophenyl)carbamoyl)phenyl)amino)-5-fluoropyrimidin-2-yl)amino)benzoic acid (107.36 mg, 224.67 mol) in DMF (1 mL) was added HATU (105.14 mg, 276.51 μmol) and DIPEA (134.02 mg, 1.04 mmol) at 20° C. After stirring for 10 min, a solution of 3-((4-(4-(((1R,5S,6s)-6-amino-3-azabicyclo[3.1.0]hexan-3-yl)methyl)piperidin-1-yl)phenyl)amino)piperidine-2,6-dione (150 mg, 345.64 μmol, HCl) in DMF (1 mL) with DIPEA (742.00 mg, 5.74 mmol, 1.00 mL) was added at 20° C. The resulting mixture was stirred at 20° C. for 1 h. LCMS showed 60% peak with desired mass was detected. The reaction mixture was poured into water and filtered. The filter cake was collected and dried. The residue was purified by prep-HPLC (column: Phenomenex Luna C18 150*25 mm*10 um; mobile phase: [water(TFA)-ACN]; gradient: 23%-43% B over 7 min) and lyophilized to afford N-(2-chlorophenyl)-4-[[2-[4-[[(1R,5S)-3-[[1-[4-[(2,6-dioxo-3-piperidyl)amino]phenyl]-4-piperidyl]methyl]-3-azabicyclo[3.1.0]hexan-6-yl]carbamoyl]anilino]-5-fluoro-pyrimidin-4-yl]amino]benzamide (31.1 mg, 24.09 μmol, 6.97% yield, 92.9% purity, 3TFA) as a gray solid. MS(M+H)⁺=857.3.

¹H NMR (400 MHz, DMSO-d₆) δ=10.84 (s, 1H), 9.95 (s, 1H), 9.78 (s, 1H), 9.67 (s, 1H), 9.50-9.31 (m, 1H), 8.47-8.32 (m, 1H), 8.26 (d, J=3.5 Hz, 1H), 8.07-7.96 (m, 4H), 7.82-7.71 (m, 4H), 7.65 (d, J=6.9 Hz, 1H), 7.57 (dd, J=1.1, 8.0 Hz, 1H), 7.46-7.26 (m, 4H), 6.77 (br d, J=8.8 Hz, 2H), 4.39 (br dd, J=4.4, 11.6 Hz, 1H), 4.09-4.13 (m, 1H), 3.90-3.80 (m, 1H), 3.57-3.51 (m, 2H), 3.47-3.40 (m, 2H), 3.33-3.28 (m, 1H), 3.24-3.04 (m, 2H), 2.98-2.84 (m, 1H), 2.80-2.69 (m, 1H), 2.64-2.56 (m, 2H), 2.18-2.11 (m, 1H), 2.11-1.99 (m, 5H), 1.97-1.88 (m, 1H), 1.71-1.50 (m, 2H).

### Example 41. Synthesis of N-(2-chlorophenyl)-4-((2-((4-(((3aR,5s,6aS)-2-((1-(4-((2,6-dioxopiperidin-3-yl)amino) phenyl)piperidin-4-yl)methyl)octahydrocyclopenta[c]pyrrol-5-yl)carbamoyl)phen yl)amino)-5-fluoropyrimidin-4-yl)amino)benzamide (Compound 41)

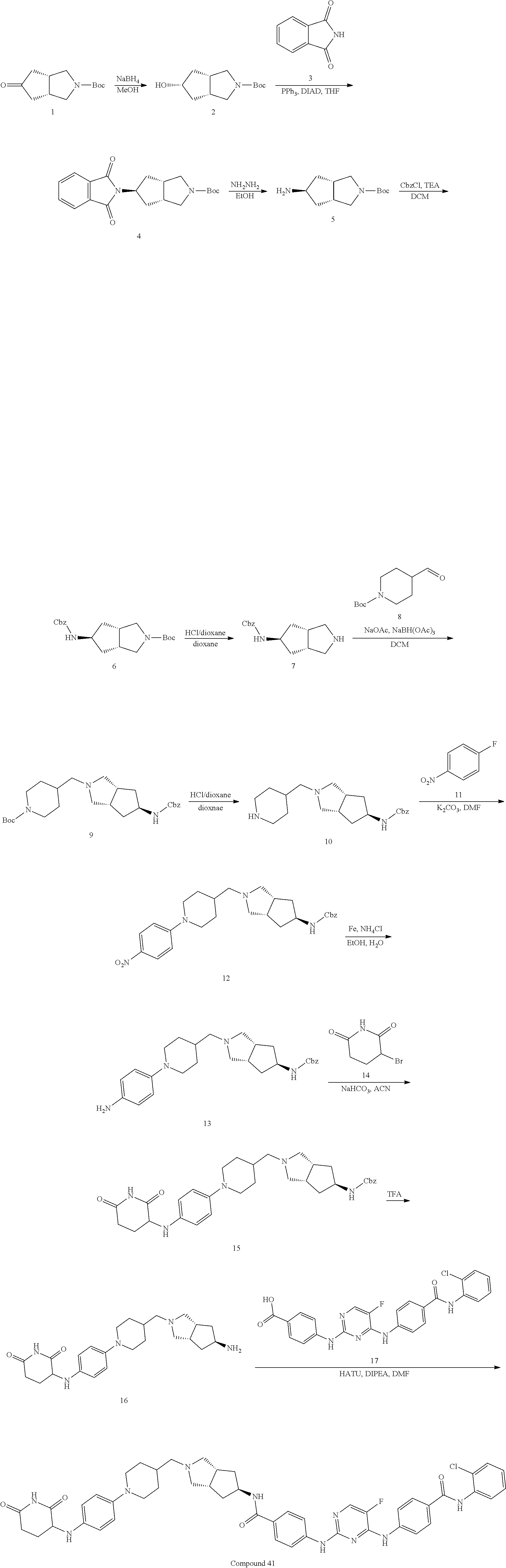

### Step 1. Synthesis of tert-butyl (3aR,5r,6aS)-5-hydroxyhexahydrocyclopenta[c]pyrrole-2(1H)-carboxylate (2)

To a solution of tert-butyl (3aR,6aS)-5-oxohexahydrocyclopenta[c]pyrrole-2(1H)-carboxylate (2 g, 8.88 mmol) in MeOH (45 mL) was added NaBH₄ (1.01 g, 26.63 mmol) in portion at 0° C. The mixture was stirred at 0° C. for 1 h. TLC (petroleum ether:EtOAc=1:2) indicated the starting material was consumed completely and one major new spot with larger polarity was detected. The reaction mixture was quenched by addition 1N HCl (50 mL), diluted with H₂O (100 mL) extracted with EtOAc (50 mL×3). The combined organic layers were washed with brine (30 mL×2), dried over Na₂SO₄ concentrated in vacuum to afford tert-butyl (3aR,5r,6aS)-5-hydroxyhexahydrocyclopenta[c]pyrrole-2(1H)-carboxylate (2 g, 8.80 mmol, 99.11% yield) as a yellow oil.

### Step 2. Synthesis of tert-butyl (3aR,5s,6aS)-5-(1,3-dioxoisoindolin-2-yl)hexahydrocyclopenta[c]pyrrole-2(1H)-carboxylate (4)

To a solution of tert-butyl (3aR,5r,6aS)-5-hydroxyhexahydrocyclopenta[c]pyrrole-2(1H)-carboxylate (1 g, 4.40 mmol), PPh₃ (1.50 g, 5.72 mmol) and isoindoline-1,3-dione (841.49 mg, 5.72 mmol) in THF (30 mL) was added DIAD (1.16 g, 5.72 mmol, 1.11 mL) dropwise at 0° C. under N₂ atmosphere. The resulting solution was warmed to 20° C. and stirred for 1 h. LCMS showed a main peak with desired mass. The reaction was quenched with MeOH (30 mL) and concentrated under reduced pressure. The residue was purified by flash silica gel chromatography (Biotage; 20 g SepaFlash® Silica Flash Column, eluent of 0-30% EtOAc/petroleum ether, gradient @ 80 mL/min) twice to afford tert-butyl (3aR,5s,6aS)-5-(1,3-dioxoisoindolin-2-yl)hexahydrocyclopenta[c]pyrrole-2(1H)-carboxylate (1.5 g, 4.21 mmol, 95.66% yield) as a white solid. MS(M+Na)⁺=379.2.

### Step 3. Synthesis of tert-butyl (3aR,5s,6aS)-5-aminohexahydrocyclopenta[c]pyrrole-2(1H)-carboxylate (5)

To a solution of tert-butyl (3aR,5s,6aS)-5-(1,3-dioxoisoindolin-2-yl)hexahydrocyclopenta[c]pyrrole-2(1H)-carboxylate (1 g, 2.81 mmol) in EtOH (10 mL) was added NH₂—NH₂—H₂O (877.84 mg, 14.03 mmol, 850.62 μL, 80% purity), the resulting mixture was stirred at 80° C. for 2 h. LCMS showed a peak (~70%) with desired mass. The reaction mixture was filtered and washed with EtOH (20 mL). The filtrate was concentrated under reduced pressure to afford tert-butyl (3aR,5s,6aS)-5-aminohexahydrocyclopenta[c]pyrrole-2(1H)-carboxylate (600 mg, 2.65 mmol, 94.49% yield) as a yellow oil, which was used into the next step directly. MS(M+H)⁺=227.2.

### Step 4. Synthesis of tert-butyl (3aR,5s,6aS)-5-(((benzyloxy)carbonyl)amino)hexahydrocyclopenta [c]pyrrole-2(1H)-carboxylate (6)

To a solution of tert-butyl (3aR,5s,6aS)-5-aminohexahydrocyclopenta[c]pyrrole-2(1H)-carboxylate (600 mg, 2.65 mmol) in DCM (10 mL) were added TEA (804.82 mg, 7.95 mmol, 1.11 mL) and CbzCl (542.72 mg, 3.18 mmol). The resulting mixture was stirred at 20° C. for 16 h. LCMS showed a main peak with desired mass. The reaction mixture was diluted with H₂O (20 mL) and the layers were separated. The aqueous phase was extracted with EtOAc (20 mL×3). The combined organic layers were washed with brine (20 mL×2), dried over Na₂SO₄ and filtered. The filtrate was concentrated under reduced pressure. The residue was purified by flash silica gel chromatography (Biotage; 20 g SepaFlash® Silica Flash Column, eluent of 0-30% EtOAc/petroleum ether, gradient @ 80 mL/min) and further purified by prep-HPLC (column: Waters Xbridge C18 150*50 mm*10 um; mobile phase: [water (NH₄HCO₃)-ACN]; gradient: 39%-69% B over 10 min) to afford tert-butyl (3aR,5s,6aS)-5-(((benzyloxy) carbonyl)amino)hexahydrocyclopenta[c]pyrrole-2(1H)-carboxylate (300 mg, 832.30 mol, 60.00% yield) as a yellow oil. MS(M+H)⁺=361.2.

### Step 5. Synthesis of benzyl ((3aR,5s,6aS)-octahydrocyclopenta[c]pyrrol-5-yl)carbamate (7)

To a solution of tert-butyl (3aR,5s,6aS)-5-(((benzyloxy)carbonyl)amino)hexahydrocyclopenta[c]pyrrole-2(1H)-carboxylate (300 mg, 832.30 μmol) in dioxane (5 mL) was added HCl/dioxane (4 M, 5 mL), the mixture was stirred at 20° C. for 0.5 h. LCMS showed a main peak with desired mass. The mixture was concentrated in vacuum to afford benzyl ((3aR,5s,6aS)-octahydrocyclopenta[c]pyrrol-5-yl)carbamate (250 mg, crude, HCl salt) as a white solid. MS(M+H)⁺=261.2.

### Step 6. Synthesis of tert-butyl 4-(((3aR,5s,6aS)-5-(((benzyloxy)carbonyl)amino) hexahydrocyclopenta[c]pyrrol-2(1H)-yl)methyl)piperidine-1-carboxylate (9)

To a solution of benzyl ((3aR,5s,6aS)-octahydrocyclopenta[c]pyrrol-5-yl)carbamate (250 mg, 842.34 μmol, HCl salt), tert-butyl 4-formylpiperidine-1-carboxylate (179.65 mg, 842.34 μmol) in DCM (5 mL) was added NaOAc (138.20 mg, 1.68 mmol). After stirring at 20° C. for 0.5 h, NaBH(OAc)₃ (535.58 mg, 2.53 mmol) was added. The resulting mixture was stirred at 20° C. for 16 h. LCMS showed a main peak with desired mass. The reaction mixture was quenched by addition NaHCO₃ solution (20 mL) and extracted with EtOAc (10 mL×3). The combined organic layers were washed with brine (20 mL×2), dried over Na₂SO₄, filtered. The filtrate was concentrated in vacuum. The residue was purified by flash silica gel chromatography (Biotage; 12 g SepaFlash® Silica Flash Column, eluent of 50-100% EtOAc/petroleum ether to 10% MeOH in EtOAc, gradient @ 80 mL/min) to afford tert-butyl 4-(((3aR,5s,6aS)-5-(((benzyloxy)carbonyl)amino)hexahydrocyclopenta[c]pyrrol-2(1H)-yl)methyl)piperidine-1-carboxylate (400 mg) as a yellow oil. MS(M+H)⁺=458.3.

### Step 7. Synthesis of benzyl ((3aR,5s,6aS)-2-(piperidin-4-ylmethyl)octahydrocyclopenta[c]pyrrol-5-yl)carbamate (10)

To a solution of tert-butyl 4-(((3aR,5s,6aS)-5-(((benzyloxy)carbonyl)amino)hexahydrocyclopenta [c]pyrrol-2(1H)-yl)methyl)piperidine-1-carboxylate (200 mg, 437.06 mol) in dioxane (5 mL) was added HCl/dioxane (4 M, 5 mL), the resulting mixture was stirred at 20° C. for 0.5 h. LCMS showed the starting material was consumed completely and a peak with desired mass. The mixture was concentrated in vacuum to afford benzyl ((3aR,5s,6aS)-2-(piperidin-4-ylmethyl) octahydrocyclopenta[c]pyrrol-5-yl)carbamate (170 mg, 431.53 μmol, 98.73% yield, HCl salt) as a white solid. MS(M+H)⁺=358.3.

### Step 8. Synthesis of benzyl ((3aR,5s,6aS)-2-((1-(4-nitrophenyl)piperidin-4-yl)methyl) octahydrocyclopenta[c]pyrrol-5-yl)carbamate (12)

To a solution of benzyl ((3aR,5s,6aS)-2-(piperidin-4-ylmethyl)octahydrocyclopenta [c]pyrrol-5-yl)carbamate (170 mg, 431.53 μmol, HCl salt), 1-fluoro-4-nitrobenzene (100.47 mg, 712.02 μmol, 75.54 μL) in DMF (5 mL) was added K₂CO₃ (238.56 mg, 1.73 mmol). The resulting mixture was stirred at 40° C. for 16 h. LCMS showed a peak (39%) with desired mass. The mixture was diluted with water (20 mL) and extracted with EtOAc (20 mL×3). The combined organic layers were washed with brine (10 mL×2), dried over Na₂SO₄, filtered. The filtrate was concentrated in vacuum and the residue was purified by flash silica gel chromatography (Biotage; 10 g SepaFlash® Silica Flash Column, eluent of 50-100% EtOAc/petroleum ether to 10% MeOH in EtOAc, gradient @ 50 mL/min) to afford benzyl ((3aR,5s,6aS)-2-((1-(4-nitrophenyl)piperidin-4-yl)methyl)octahydrocyclopenta[c]pyrrol-5-yl)carbamate (160 mg, 334.32 μmol, 77.47% yield) as a yellow oil. MS(M+H)⁺=479.3.

### Step 9. Synthesis of benzyl ((3aR,5s,6aS)-2-((1-(4-aminophenyl)piperidin-4-yl)methyl) octahydrocyclopenta[c]pyrrol-5-yl)carbamate (13)

To a solution of benzyl ((3aR,5s,6aS)-2-((1-(4-nitrophenyl)piperidin-4-yl)methyl) octahydrocyclopenta[c]pyrrol-5-yl)carbamate (160 mg, 334.32 μmol) in EtOH (5 mL) and H₂O (1 mL) were added Fe (93.35 mg, 1.67 mmol) and NH₄Cl (89.42 mg, 1.67 mmol). The resulting mixture was stirred at 80° C. for 2 h. LCMS showed a peak (58%) with desired mass. The mixture was filtered through a pad of celite and the filtrate was concentrated in vacuum. The residue was diluted with NaHCO₃ solution (10 mL) and extracted with EtOAc (10 mL×3). The combined organic layers were washed with brine (10 mL×2), dried over Na₂SO₄, filtered. The filtrate was concentrated in vacuum to afford benzyl ((3aR,5s,6aS)-2-((1-(4-aminophenyl)piperidin-4-yl)methyl)octahydrocyclopenta[c]pyrrol-5-yl)carbamate (140 mg, crude) as brown oil. MS(M+H)⁺=449.3.

### Step 10. Synthesis of benzyl ((3aR,5s,6aS)-2-((1-(4-((2,6-dioxopiperidin-3-yl)amino)phenyl)piperidin-4-yl)methyl) octahydrocyclopenta[c]pyrrol-5-yl)carbamate (15)

To a solution of benzyl ((3aR,5s,6aS)-2-((1-(4-aminophenyl)piperidin-4-yl)methyl) octahydrocyclopenta[c]pyrrol-5-yl)carbamate (100 mg, 222.92 mol), 3-bromopiperidine-2,6-dione (42.80 mg, 222.92 μmol) in ACN (1 mL) was added NaHCO₃ (93.64 mg, 1.11 mmol). The resulting mixture was stirred at 80° C. for 16 h. LCMS showed a peak (33%) with desired mass. The mixture was diluted with water (10 mL) and extracted with EtOAc (5 mL×3). The combined organic layers were washed with brine (10 mL×2), dried over Na₂SO₄, filtered. The filtrate was concentrated in vacuum. The residue was purified by flash silica gel chromatography (Biotage; 12 g SepaFlash® Silica Flash Column, eluent of 0~20% MeOH in EtOAc, gradient @ 80 mL/min to afford benzyl ((3aR,5s,6aS)-2-((1-(4-((2,6-dioxopiperidin-3-yl)amino)phenyl)piperidin-4-yl)methyl) octahydrocyclopenta[c]pyrrol-5-yl)carbamate (120 mg) as a brown oil. MS(M+H)⁺=560.3.

### Step 11. Synthesis of 3-((4-(4-(((3aR,5s,6aS)-5-aminohexahydrocyclopenta[c]pyrrol-2(1H)-yl)methyl)piperidin-1-yl)phenyl)amino)piperidine-2,6-dione (16)

A mixture of benzyl ((3aR,5s,6aS)-2-((1-(4-((2,6-dioxopiperidin-3-yl)amino)phenyl)piperidin-4-yl) methyl)octahydrocyclopenta[c]pyrrol-5-yl)carbamate (100 mg, 178.67 mol) in TFA (2 mL) was stirred at 40° C. for 1 h. LCMS showed the starting material was consumed completely and a peak (50%) with desired mass. The reaction mixture was concentrated in vacuum to afford 3-((4-(4-(((3aR,5s,6aS)-5-aminohexahydrocyclopenta[c]pyrrol-2(1H)-yl)methyl)piperidin-1-yl)phenyl)amino)piperidine-2,6-dione (90 mg, crude, TFA salt) as brown oil. MS(M+H)⁺=426.3.

### Step 12. Synthesis of N-(2-chlorophenyl)-4-((2-((4-(((3aR,5s,6aS)-2-((1-(4-((2,6-dioxopiperidin-3-yl)amino)phenyl)piperidin-4-yl)methyl)octahydrocyclopenta[c]pyrrol-5-yl)carbamoyl)phenyl)amino)-5-fluoropyrimidin-4-yl)amino)benzamide (Compound 41)

To a solution of 4-((4-((4-((2-chlorophenyl)carbamoyl)phenyl)amino)-5-fluoropyrimidin-2-yl) amino)benzoic acid (79.71 mg, 166.79 mol) in DMF (2 mL) were added HATU (76.10 mg, 200.15 μmol), DIPEA (107.78 mg, 833.97 μmol). After stirring at 20° C. for 0.5 h, 3-((4-(4-(((3aR,5s,6aS)-5-aminohexahydrocyclopenta[c]pyrrol-2(1H)-yl)methyl)piperidin-1-yl)phenyl)amino)piperidine-2,6-dione (90 mg, 166.79 μmol, TFA salt) was added and the resulting mixture was stirred at 20° C. for 1 h. LCMS showed a peak (20%) with desired mass. The reaction mixture was diluted with water (10 mL) and extracted with EtOAc (10 mL×3). The combined organic layers were washed with brine (10 mL×2), dried over Na₂SO₄, filtered. The filtrate was concentrated in vacuum. The crude product was purified by reversed-phase HPLC (column: Phenomenex luna C18 150*25 mm*10 um; mobile phase: [water (TFA)-ACN]; gradient: 20%-50% B over 10 min). The eluent was lyophilized to afford N-(2-chlorophenyl)-4-((2-((4-(((3aR,5s,6aS)-2-((1-(4-((2,6-dioxopiperidin-3-yl)amino)phenyl)piperidin-4-yl)methyl)octahydrocyclopenta[c]pyrrol-5-yl)carbamoyl)phenyl)amino)-5-fluoropyrimidin-4-yl)amino)benzamide (11.2 mg, 11.38 μmol, 6.83% yield, 90% purity) as a green solid. MS(M+H)⁺=885.3.

¹H NMR (400 MHz, DMSO-d₆) δ=10.83 (s, 1H), 9.94 (s, 1H), 9.76 (s, 1H), 9.63 (s, 1H), 8.25 (d, J=3.5 Hz, 1H), 8.17 (d, J=7.8 Hz, 1H), 8.05-7.98 (m, 4H), 7.80-7.72 (m, 4H), 7.67-7.62 (m, 1H), 7.59-7.55 (m, 1H), 7.49-7.24 (m, 5H), 7.15-6.94 (m, 1H), 6.77 (d, J=8.6 Hz, 1H), 4.62-4.49 (m, 1H), 4.48-4.33 (m, 1H), 3.87-3.78 (m, 2H), 3.60-3.53 (m, 4H), 2.84-2.71 (m, 5H), 2.64-2.57 (m, 2H), 2.14-2.00 (m, 3H), 1.97-1.82 (m, 5H), 1.78-1.55 (m, 4H).

### Example 42. Synthesis of N-(2-chlorophenyl)-4-((2-((4-(4-((6-(4-((2,6-dioxopiperidin-3-yl)amino)phenyl)-2,6-diazaspiro[3.3]heptan-2-yl)methyl)piperidine-1-carbonyl)phenyl)amino)-5-fluoropyrimidin-4-yl)amino)benzamide (Compound 42)

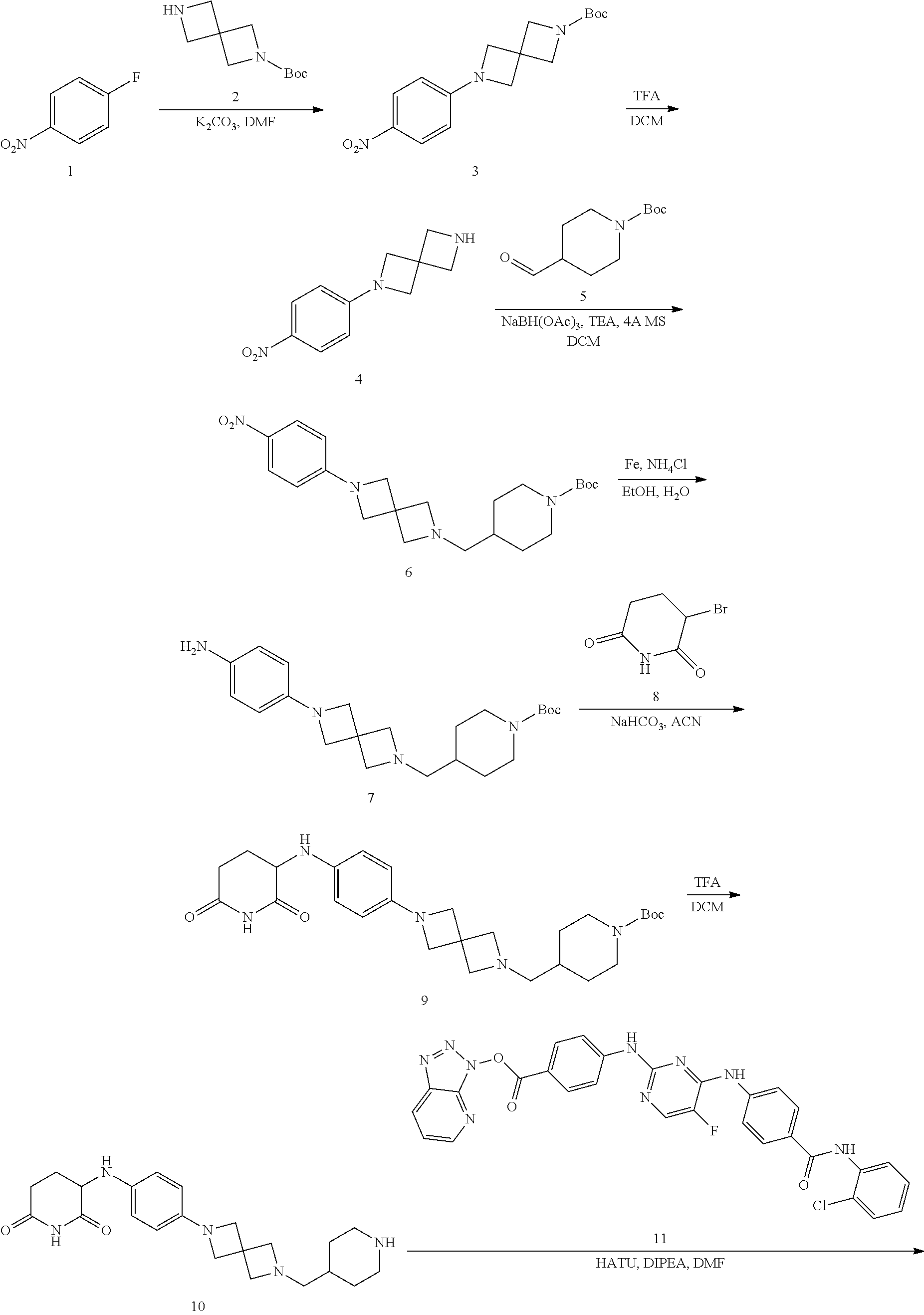

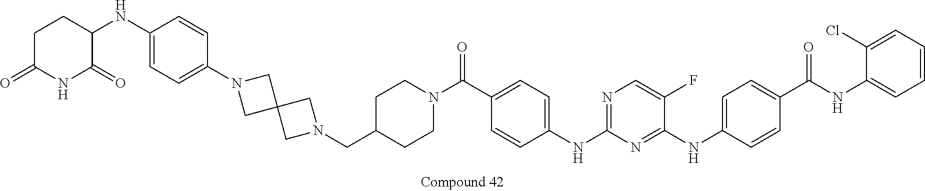 [SMILES: O=C1CCC(Nc2ccc(N3CC4(CN(CC5CCN(C(=O)c6ccc(Nc7ncc(F)c(Nc8ccc(C(=O)Nc9ccccc9Cl)cc8)n7)cc6)CC5)C4)C3)cc2)C(=O)N1]

### Step 1. Synthesis of tert-butyl 6-(4-nitrophenyl)-2,6-diazaspiro[3.3]heptane-2-carboxylate (3)

To a solution of tert-butyl 2,6-diazaspiro[3.3]heptane-2-carboxylate (4.3 g, 14.92 mmol, H₂C₂O₄) and 1-fluoro-4-nitrobenzene (4.21 g, 29.83 mmol, 3.16 mL) in DMF (5 mL) was added K₂CO₃ (6.18 g, 44.75 mmol). The mixture was stirred at 25° C. for 16 hr. LCMS showed tert-butyl 2,6-diazaspiro[3.3]heptane-2-carboxylate was consumed completely. The reaction mixture was diluted with H₂O (50 mL) and EtOAc (50 mL). The resulting mixture was filtered. The filter cake was collected and dried to afford part of the desired product tert-butyl 6-(4-nitrophenyl)-2,6-diazaspiro[3.3]heptane-2-carboxylate (3.6 g, 11.27 mmol, 75.58% yield, 100% purity) as a yellow solid. The filtrate was extracted with EtOAc (30 mL×2). The combined organic layers were washed with brine (40 mL), dried over Na₂SO₄, filtered and concentrated under reduced pressure. The residue was purified by flash silica gel chromatography (ISCO®; 20 g SepaFlash® Silica Flash Column, eluent of 0~20% EtOAc/petroleum ether, gradient @ 80 mL/min) to afford tert-butyl 6-(4-nitrophenyl)-2,6-diazaspiro[3.3]heptane-2-carboxylate (1.9 g, 3.69 mmol, 24.73% yield, 62% purity) as a yellow solid. MS(M+H)⁺=320.3.

### Step 2. Synthesis of 2-(4-nitrophenyl)-2,6-diazaspiro[3.3]heptane (4)

To a solution of tert-butyl 6-(4-nitrophenyl)-2,6-diazaspiro[3.3]heptane-2-carboxylate (3.5 g, 10.96 mmol) in DCM (35 mL) was added TFA (16.88 g, 148.08 mmol, 11 mL). The mixture was stirred at 25° C. for 1 hr. LCMS showed tert-butyl 6-(4-nitrophenyl)-2,6-diazaspiro[3.3]heptane-2-carboxylate was consumed completely. The reaction mixture was concentrated under reduced pressure to afford 2-(4-nitrophenyl)-2,6-diazaspiro[3.3]heptane (3.6 g, 10.80 mmol, 98.56% yield, TFA) as a yellow oil. MS(M+H)⁺=220.1.

### Step 3. Synthesis of tert-butyl 4-((6-(4-nitrophenyl)-2,6-diazaspiro[3.3]heptan-2-yl)methyl)piperidine-1-carboxylate (6)

To a solution of 2-(4-nitrophenyl)-2,6-diazaspiro[3.3]heptane (3.6 g, 10.80 mmol, TFA) and tert-butyl 4-formylpiperidine-1-carboxylate (2.30 g, 10.80 mmol) in DCM (36 mL) was added TEA (3.28 g, 32.41 mmol, 4.51 mL) and 4 Å MS (800 mg). After stirring at 25° C. for 0.5 hr, NaBH(OAc)₃ (6.87 g, 32.41 mmol) was added in portions and the mixture was stirred at 25° C. for 16 hr. LCMS showed 2-(4-nitrophenyl)-2,6-diazaspiro[3.3]heptane was consumed completely. The reaction mixture was diluted with H₂O (100 mL) and then extracted with EtOAc (60 mL×2). The combined organic layers were dried over Na₂SO₄, filtered and concentrated under reduced pressure. The residue was purified by flash silica gel chromatography (ISCO®; 40 g SepaFlash® Silica Flash Column, eluent of 0~20% MeOH/EtOAc @ 80 mL/min) to afford tert-butyl 4-((6-(4-nitrophenyl)-2,6-diazaspiro[3.3]heptan-2-yl)methyl)piperidine-1-carboxylate (3.33 g, 7.99 mmol, 74.01% yield) as a yellow solid. MS(M+H)⁺=417.4.

### Step 4. Synthesis of tert-butyl 4-((6-(4-aminophenyl)-2,6-diazaspiro[3.3]heptan-2-yl)methyl)piperidine-1-carboxylate (7)

To a solution of tert-butyl 4-((6-(4-nitrophenyl)-2,6-diazaspiro[3.3]heptan-2-yl)methyl)piperidine-1-carboxylate (2.2 g, 5.28 mmol) in EtOH (44 mL) and H₂O (11 mL) were added NH₄Cl (2.83 g, 52.82 mmol) and Fe (1.47 g, 26.41 mmol). The mixture was stirred at 80° C. for 1 hr. LC-MS showed the starting material was consumed completely. The reaction mixture was diluted with H₂O (150 mL) and extracted with EtOAc (60 mL×3). The combined organic layers were dried over Na₂SO₄, filtered and concentrated under reduced pressure to afford tert-butyl 4-((6-(4-aminophenyl)-2,6-diazaspiro[3.3]heptan-2-yl)methyl)piperidine-1-carboxylate (1 g, 2.59 mmol, 48.98% yield) as a brown solid. MS(M+H)⁺=387.3.

### Step 5. Synthesis of tert-butyl 4-((6-(4-((2,6-dioxopiperidin-3-yl)amino)phenyl)-2,6-diazaspiro[3.3]heptan-2-yl)methyl)piperidine-1-carboxylate (9)

To a solution of tert-butyl 4-((6-(4-aminophenyl)-2,6-diazaspiro[3.3]heptan-2-yl)methyl)piperidine-1-carboxylate (210 mg, 543.30 μmol) and 3-bromopiperidine-2,6-dione (104.32 mg, 543.30 μmol) in ACN (1 mL) was added NaHCO₃ (264.71 mg, 3.15 mmol). The resulting mixture was stirred at 60° C. for 3 hr. LCMS showed 13% of tert-butyl 4-((6-(4-aminophenyl)-2,6-diazaspiro[3.3]heptan-2-yl)methyl)piperidine-1-carboxylate remained. The reaction mixture was diluted with H₂O (10 mL) and extracted with EtOAc (10 mL×2). The combined organic layers were dried over Na₂SO₄, filtered and concentrated under reduced pressure. The residue was purified by flash silica gel chromatography (ISCO®; 20 g SepaFlash® Silica Flash Column, eluent of 0~20% MeOH in EtOAc @ 60 mL/min) to afford tert-butyl 4-((6-(4-((2,6-dioxopiperidin-3-yl)amino)phenyl)-2,6-diazaspiro[3.3]heptan-2-yl)meth yl)piperidine-1-carboxylate (127 mg, 222.03 μmol, 40.87% yield, 87% purity) as a black solid. MS(M+H)⁺=498.3.

### Step 6. Synthesis of 3-((4-(6-(piperidin-4-ylmethyl)-2,6-diazaspiro[3.3]heptan-2-yl)phenyl)amino)piperidine-2,6-dione (10)

To a solution of tert-butyl 4-((6-(4-((2,6-dioxopiperidin-3-yl)amino)phenyl)-2,6-diazaspiro[3.3]heptan-2-yl)methyl)piperidine-1-carboxylate (120 mg, 209.80 μmol) in DCM (1.2 mL) was added TFA (614.00 mg, 5.38 mmol, 0.4 mL). The mixture was stirred at 25° C. for 0.5 hr. LCMS showed the starting material was consumed completely. The reaction mixture was concentrated under reduced pressure to afford 3-((4-(6-(piperidin-4-ylmethyl)-2,6-diazaspiro[3.3]heptan-2-yl)phenyl)amino)piperidine-2,6-dione (107 mg, 209.17 μmol, 99.70% yield, TFA) as a black oil. MS(M+H)⁺=398.3.

### Step 7. Synthesis of N-(2-chlorophenyl)-4-((2-((4-(4-((6-(4-((2,6-dioxopiperidin-3-yl)amino)phenyl)-2,6-diazaspiro[3.3]heptan-2-yl)methyl)piperidine-1-carbonyl)phenyl)amino)-5-fluoropyrimidin-4-yl)amino)benzamide (Compound 42)

To a solution of 3H-[1,2,3]triazolo[4,5-b]pyridin-3-yl 4-((4-((4-((2-chlorophenyl)carbamoyl)phenyl)amino)-5-fluoropyrimidin-2-yl)amino)benzoate (86.02 mg, 134.23 mol) and 3-((4-(6-(piperidin-4-ylmethyl)-2,6-diazaspiro[3.3]heptan-2-yl)phenyl)amino)piperidine-2,6-dione (103.00 mg, 201.35 μmol, TFA) in DMF (1 mL) was added DIPEA (173.49 mg, 1.34 mmol, 233.81 μL). The mixture was stirred at 25° C. for 16 hr. LCMS showed 3H-[1,2,3]triazolo[4,5-b]pyridin-3-yl 4-((4-((4-((2-chlorophenyl)carbamoyl)phenyl)amino)-5-fluoropyrimidin-2-yl)amino)benzoate was consumed completely. The reaction mixture was diluted with H₂O (20 mL) and then extracted with EtOAc (10 mL×2). The combined organic layers were washed with brine (10 mL), dried over Na₂SO₄, filtered and concentrated under reduced pressure. The residue was purified by flash silica gel chromatography (ISCO®; 20 g SepaFlash® Silica Flash Column, eluent of 0~20% MeOH/EtOAc @ 50 mL/min). The impure product was purified by prep-HPLC (column: Phenomenex luna C18 150*25 mm*10 um; mobile phase: [water (TFA)-ACN]; gradient: 15%-45% B over 10 min) and lyophilized to afford N-(2-chlorophenyl)-4-((2-((4-(4-((6-(4-((2,6-dioxopiperidin-3-yl)amino)phenyl)-2,6-diazaspiro[3.3]heptan-2-yl)methyl)piperidine-1-carbonyl)phenyl)amino)-5-fluoropyrimidin-4-yl)amino)benzamide (11.2 mg, 9.80 μmol, 7.30% yield, 85% purity, TFA) as a white solid. MS(M+H)⁺=857.4.

¹H NMR (400 MHz, DMSO-d₆) δ=10.86 (s, 1H), 9.95 (s, 1H), 9.75 (s, 1H), 9.63-9.52 (m, 2H), 8.23 (d, J=3.6 Hz, 1H), 8.10-8.00 (m, 4H), 7.75 (d, J=8.4 Hz, 2H), 7.67-7.61 (m, 1H), 7.59-7.55 (m, 1H), 7.45-7.37 (m, 1H), 7.32-7.28 (m, 3H), 6.69-6.62 (m, 2H), 6.41-6.34 (m, 2H), 4.38-4.31 (m, 2H), 4.27-4.16 (m, 4H), 3.93-3.89 (m, 2H), 3.82-3.78 (m, 3H), 3.13-3.04 (m, 3H), 2.64-2.56 (m, 1H), 2.46-2.39 (m, 1H), 2.07-1.98 (m, 1H), 1.89-1.78 (m, 2H), 1.73-1.60 (m, 2H), 1.27-1.05 (m, 3H).

### Example 43. Synthesis of N-(2-chlorophenyl)-4-((2-((4-((4-(2-(4-(4-((2,6-dioxopiperidin-3-yl)amino)phenyl)piperazin-1-yl)ethyl)piperidin-1-yl)carbamoyl)phenyl)amino)-5-fluoropyrimidin-4-yl)amino)benzamide (Compound 43)

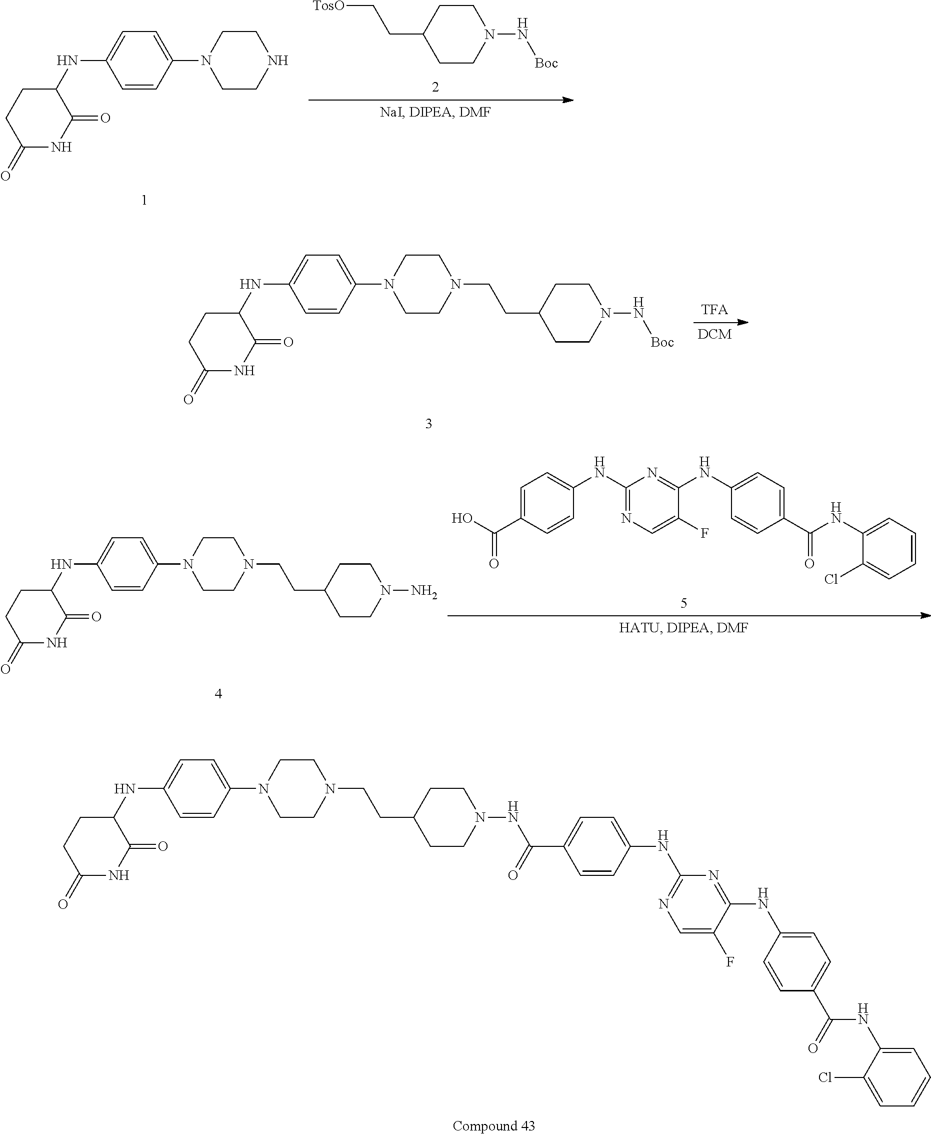

### Step 1. Synthesis of tert-butyl (4-(2-(4-(4-((2,6-dioxopiperidin-3-yl)amino)phenyl)piperazin-1-yl)ethyl)piperidin-1-yl)carbamate (3)

To a solution of 3-((4-(piperazin-1-yl)phenyl)amino)piperidine-2,6-dione (250 mg, 769.69 μmol, HCl) and 2-(1-((tert-butoxycarbonyl)amino)piperidin-4-yl)ethyl 4-methylbenzenesulfonate (460.10 mg, 1.15 mmol) in DMF (2 mL) was added NaI (46.15 mg, 307.88 mol) and DIPEA (795.82 mg, 6.16 mmol, 1.07 mL). The mixture was stirred at 60° C. for 16 hrs. LCMS showed 79% of 3-((4-(piperazin-1-yl)phenyl)amino)piperidine-2,6-dione remained and 10% of desired mass. Another portion of 2-(1-((tert-butoxycarbonyl)amino)piperidin-4-yl)ethyl 4-methylbenzenesulfonate (306.73 mg, 769.69 mol) was added and the resulting mixture was stirred at 60° C. for another 5 hrs. LCMS showed 27% of 3-((4-(piperazin-1-yl)phenyl)amino)piperidine-2,6-dione remained and 36% of desired mass. The reaction mixture was poured into water (20 mL) and extracted with EtOAc (10 mL×3). The combined organic layers were washed with brine (10 mL), dried over Na₂SO₄, filtered and concentrated under reduced pressure. The crude product was triturated with MTBE at 20° C. for 60 min to afford tert-butyl (4-(2-(4-(4-((2,6-dioxopiperidin-3-yl)amino)phenyl)piperazin-1-yl)ethyl)piperidin-1-yl)carbamate (600 mg, crude) as a black brown solid. MS(M+H)⁺=515.4.

### Step 2. Synthesis of 3-((4-(4-(2-(1-aminopiperidin-4-yl)ethyl)piperazin-1-yl)phenyl)amino)piperidine-2,6-dione (4)

To a solution of tert-butyl (4-(2-(4-(4-((2,6-dioxopiperidin-3-yl)amino)phenyl)piperazin-1-yl)ethyl)piperidin-1-yl)carbamate (200 mg, 388.61 μmol) in DCM (2 mL) was added TFA (921.00 mg, 8.08 mmol, 0.6 mL). The mixture was stirred at 25° C. for 1 hr. LCMS showed the starting material was consumed completely and desired mass. The reaction mixture was concentrated under reduced pressure to afford 3-((4-(4-(2-(1-aminopiperidin-4-yl)ethyl)piperazin-1-yl)phenyl)amino)piperidine-2,6-dione (200 mg, 378.38 μmol, 97.37% yield, TFA) as a brown oil. MS(M+H)⁺=415.3.

### Step 3. Synthesis of N-(2-chlorophenyl)-4-((2-((4-((4-(2-(4-(4-((2,6-dioxopiperidin-3-yl)amino)phenyl)piperazin-1-yl)ethyl)piperidin-1-yl)carbamoyl)phenyl)amino)-5-fluoropyrimidin-4-yl)amino) benzamide (Compound 43)

To a solution of 4-((4-((4-((2-chlorophenyl)carbamoyl)phenyl)amino)-5-fluoropyrimidin-2-yl)amino)benzoic acid (241.09 mg, 378.38 μmol) in DMF (2 mL) was added HATU (215.81 mg, 567.57 μmol) and DIPEA (244.52 mg, 3.78 mmol, 329.54 μL). The mixture was stirred at 25° C. for 0.5 h. Then 3-((4-(4-(2-(1-aminopiperidin-4-yl)ethyl)piperazin-1-yl)phenyl)amino)piperidine-2,6-dione (200 mg, 378.38 μmol, TFA) was added and the resulting mixture was stirred at 25° C. for 16 hr. LCMS showed 17% of 4-((4-((4-((2-chlorophenyl)carbamoyl)phenyl)amino)-5-fluoropyrimidin-2-yl)amino)benzoic acid remained and 26% peak of desired mass. The reaction mixture was poured into H₂O (10 mL) and extracted with EtOAc (10 mL×3). The combined organic layers were washed with brine (10 mL), dried over Na₂SO₄, filtered and concentrated under reduced pressure to give a residue. The residue was purified by prep-TLC (SiO₂, DCM:MeOH=10:1), followed by prep-HPLC (column: Phenomenex luna C18 150*25 mm*10 um; mobile phase: [water (TFA)-ACN]; gradient: 23%-53% B over 10 min) to afford N-(2-chlorophenyl)-4-((2-((4-((4-(2-(4-(4-((2,6-dioxopiperidin-3-yl)amino)phenyl)piperazin-1-yl)ethyl)piperidin-1-yl)carbamoyl)phenyl)amino)-5-fluoropyrimidin-4-yl)amino) benzamide (8.5 mg, 7.25 μmol, 1.92% yield, 94% purity, 2TFA) as a white solid. MS(M+H)⁺=874.3.

¹H NMR (400 MHz, DMSO-d₆) δ=10.78 (s, 1H), 9.93 (s, 1H), 9.81-9.73 (m, 1H), 9.66 (s, 1H), 9.63-9.54 (m, 1H), 9.46-9.32 (m, 1H), 8.25 (d, J=3.5 Hz, 1H), 8.05-7.98 (m, 4H), 7.80-7.76 (m, 2H), 7.74-7.68 (m, 2H), 7.65 (d, J=6.6 Hz, 1H), 7.60-7.54 (m, 1H), 7.43-7.37 (m, 1H), 7.33-7.27 (m, 1H), 6.83 (br d, J=8.7 Hz, 2H), 6.66 (br d, J=8.8 Hz, 2H), 4.28-4.19 (m, 1H), 3.64-3.48 (m, 4H), 3.26-3.05 (m, 6H), 2.88-2.77 (m, 3H), 2.75-2.64 (m, 1H), 2.63-2.52 (m, 1H), 2.16-2.01 (m, 2H), 1.91-1.83 (m, 1H), 1.78-1.70 (m, 2H), 1.68-1.64 (m, 2H), 1.43-1.31 (m, 3H).

### Example 44. Synthesis of N-(2-chlorophenyl)-4-((2-((4-(2-(4-((1-(4-((2,6-dioxopiperidin-3-yl)(methyl)amino)phenyl)piperidin-4-yl)methyl)piperazin-1-yl)-2-oxoethyl)phenyl)amino)-5-fluoropyrimidin-4-yl)amino)benzamide (Compound 44)

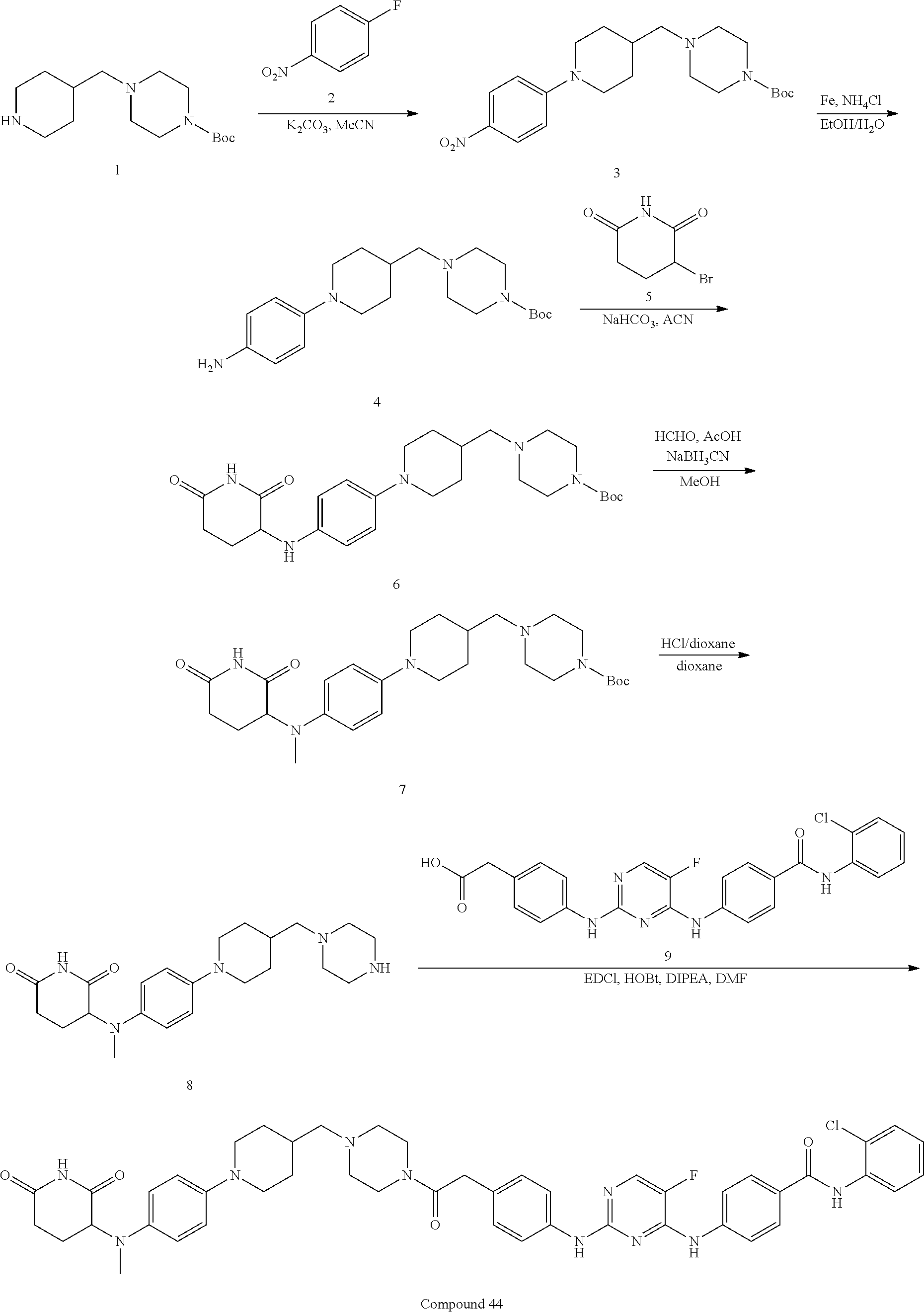

### Step 1. Synthesis of tert-butyl 4-((1-(4-nitrophenyl)piperidin-4-yl)methyl)piperazine-1-carboxylate (3)

To a solution of 1-fluoro-4-nitrobenzene (1.23 g, 8.73 mmol, 926.66 μL) and tert-butyl 4-(piperidin-4-ylmethyl)piperazine-1-carboxylate (3 g, 8.73 mmol, HOAc) in MeCN (20 mL) was added K₂CO₃ (3.62 g, 26.20 mmol). The mixture was stirred at 25° C. for 16 h. LCMS showed one main peak with desired mass. The reaction mixture was filtered and concentrated under reduced pressure to give a residue. The residue was purified by flash silica gel chromatography (ISCO®; 12 g SepaFlash® Silica Flash Column, Eluent of 0-100% EtOAc/Petroleum ether gradient @ 100 mL/min) to afford tert-butyl 4-((1-(4-nitrophenyl)piperidin-4-yl)methyl)piperazine-1-carboxylate (2.3 g, 5.69 mmol, 65.10% yield, 100% purity) as a yellow solid. MS(M+H)⁺=405.2.

### Step 2. Synthesis of tert-butyl 4-((1-(4-aminophenyl)piperidin-4-yl)methyl)piperazine-1-carboxylate (4)

To a mixture of tert-butyl 4-((1-(4-nitrophenyl)piperidin-4-yl)methyl)piperazine-1-carboxylate (2.5 g, 6.18 mmol) and NH₄Cl (1.32 g, 24.72 mmol) in EtOH (20 mL) and H₂O (20 mL) was added Fe (1.04 g, 18.54 mmol) at 80° C. The resulting mixture was stirred at 80° C. for 1 h. LCMS showed one main peak with desired mass. The reaction mixture was poured into NaHCO₃ solution (50 mL) and the mixture was extracted with EtOAc (10 mL×3). The combined organic layers were dried over Na₂SO₄ and concentrated under reduced pressure to afford tert-butyl 4-((1-(4-aminophenyl)piperidin-4-yl)methyl)piperazine-1-carboxylate (1.7 g, 3.09 mmol, 49.94% yield, 68% purity) as a yellow solid. The crude product was used into the next step without further purification. MS(M+H)⁺=375.3.

### Step 3. Synthesis of tert-butyl 4-((1-(4-((2,6-dioxopiperidin-3-yl)amino)phenyl)piperidin-4-yl) methyl)piperazine-1-carboxylate (6)

To a solution of tert-butyl 4-((1-(4-aminophenyl)piperidin-4-yl)methyl)piperazine-1-carboxylate (0.8 g, 2.14 mmol) and 3-bromopiperidine-2,6-dione (820.30 mg, 4.27 mmol) in ACN (4 mL) was added NaHCO₃ (897.22 mg, 10.68 mmol, 415.38 μL). The resulting mixture was stirred at 80° C. for 16 h. LCMS showed that the reactant 1 was consumed completely and one main peak with desired mass was detected. The reaction mixture was filtered and concentrated under reduced pressure to give a residue. The residue was purified by flash silica gel chromatography (ISCO®; 12 g SepaFlash® Silica Flash Column, Eluent of 0-100% EtOAc/Petroleum ether to 0~20% MeOH/EtOAc gradient @ 100 mL/min) to afford tert-butyl 4-((1-(4-((2,6-dioxopiperidin-3-yl)amino)phenyl)piperidin-4-yl)methyl)piperazine-1-carboxylate (560 mg, 1.00 mmol, 46.97% yield, 87% purity) as a brown solid. MS(M+H)⁺=486.4.

### Step 4. Synthesis of tert-butyl 4-((1-(4-((2,6-dioxopiperidin-3-yl)(methyl)amino)phenyl)piperidin-4-yl)methyl)piperazine-1-carboxylate (7)

A mixture of tert-butyl 4-((1-(4-((2,6-dioxopiperidin-3-yl)amino)phenyl)piperidin-4-yl) methyl)piperazine-1-carboxylate (360 mg, 741.32 μmol), HOAc (44.52 mg, 741.32 mol) and HCHO (300.80 mg, 3.71 mmol, 275.96 μL, 37% purity) in MeOH (4 mL) was stirred at 25° C. for 0.5 h. Then NaBH₃CN (232.93 mg, 3.71 mmol) was added and the mixture was stirred at 25° C. for 16 h. LCMS showed the starting material was consumed completely and the desired mass. The reaction mixture was concentrated under reduced pressure, 20 mL NaHCO₃ aq. and 30 mL H₂O was added and extracted with EtOAc (15 mL×3). The combined organic layers were washed with brine (15 mL), dried over Na₂SO₄, filtered and concentrated under reduced pressure to give a residue. The crude product was purified by flash silica gel chromatography (ISCO®; 12 g Sepa Flash® Silica Flash Column, Eluent of 0-100% EtOAc/Petroleum ether to 0~20% MeOH/EtOAc gradient @ 100 mL/min) to afford tert-butyl 4-((1-(4-((2,6-dioxopiperidin-3-yl)(methyl)amino)phenyl)piperidin-4-yl)methyl)piperazine-1-carboxylate (360 mg, 648.46 μmol, 87.47% yield, 90% purity) as a yellow solid. MS(M+H)⁺=500.4.

### Step 5. Synthesis of 3-(methyl(4-(4-(piperazin-1-ylmethyl)piperidin-1-yl)phenyl)amino)piperidine-2,6-dione (8)

To a solution of tert-butyl 4-((1-(4-((2,6-dioxopiperidin-3-yl)(methyl)amino)phenyl)piperidin-4-yl)methyl)piperazine-1-carboxylate (360 mg, 720.51 μmol) in dioxane (4 mL) was added HCl/dioxane (4 M, 4 mL). The mixture was stirred at 25° C. for 2 hr. LCMS showed that the starting material was consumed completely. The reaction mixture was concentrated under reduced pressure to afford 3-(methyl(4-(4-(piperazin-1-ylmethyl)piperidin-1-yl)phenyl)amino)piperidine-2,6-dion e (320 mg, crude, HCl) as a yellow solid. The crude product was used into the next step without further purification. MS(M+H)⁺=400.3.

### Step 6. Synthesis of N-(2-chlorophenyl)-4-((2-((4-(2-(4-((1-(4-((2,6-dioxopiperidin-3-yl)(methyl)amino)phen yl)piperidin-4-yl)methyl)piperazin-1-yl)-2-oxoethyl)phenyl)amino)-5-fluoropyrimidin-4-yl)amino)benzamide (Compound 44)

To a mixture of 2-(4-((4-((4-((2-chlorophenyl)carbamoyl)phenyl)amino)-5-fluoropyrimidin-2-yl)amino)phenyl)acetic acid (90.26 mg, 183.49 mol), 3-(methyl(4-(4-(piperazin-1-ylmethyl)piperidin-1-yl)phenyl)amino)piperidine-2,6-dion e (100 mg, 229.36 μmol, HCl) and DIPEA (148.22 mg, 1.15 mmol, 199.75 μL) in DMF (1 mL) were added EDCI (52.76 mg, 275.24 μmol) and HOBt (37.19 mg, 275.24 mol). The mixture was stirred at 25° C. for 16 h. LCMS showed one main peak with desired mass was. The reaction mixture was diluted with H₂O (30 mL) and extracted with EtOAc (10 mL×3). The combined organic layers were washed with brine (15 mL), dried over Na₂SO₄, filtered and concentrated under reduced pressure to give a residue. The residue was purified by flash silica gel chromatography (ISCO®; 4 g SepaFlash® Silica Flash Column, Eluent of 0-100% EtOAc/Petroleum ether to 0-10% MeOH/EtOAc gradient @ 100 mL/min). The crude product was purified by prep-HPLC (column: Phenomenex Luna C18 150*25 mm*10 um; mobile phase: [water (TFA)-ACN]; B %: 26%-56%, 9 min) to afford N-(2-chlorophenyl)-4-((2-((4-(2-(4-((1-(4-((2,6-dioxopiperidin-3-yl)(methyl)amino)phen yl)piperidin-4-yl)methyl)piperazin-1-yl)-2-oxoethyl)phenyl)amino)-5-fluoropyrimidin-4-yl)amino)benzamide (26.2 mg, 28.50 μmol, 12.42% yield, 95% purity) as a white solid. MS(M+H)⁺=873.2.

¹H NMR (400 MHz, DMSO-d₆) δ=10.72 (br s, 1H), 9.88 (s, 1H), 9.65 (br s, 1H), 9.28 (s, 1H), 8.18 (d, J=3.4 Hz, 1H), 8.06-8.00 (m, 2H), 8.00-7.94 (m, 2H), 7.66 (br d, J=7.9 Hz, 1H), 7.60 (br d, J=8.3 Hz, 2H), 7.56 (d, J=7.9 Hz, 1H), 7.39 (t, J=7.6 Hz, 1H), 7.32-7.25 (m, 1H), 7.12 (br d, J=8.3 Hz, 2H), 6.83-6.76 (m, 2H), 6.75-6.68 (m, 2H), 4.70 (br dd, J=4.7, 12.4 Hz, 1H), 3.64 (s, 2H), 3.55-3.45 (m, 4H), 3.45-3.37 (m, 2H), 2.85-2.75 (m, 1H), 2.74-2.66 (m, 4H), 2.48-2.40 (m, 2H), 2.30-2.16 (m, 5H), 2.12-2.08 (m, 2H), 1.88-1.79 (m, 1H), 1.78-1.68 (m, 2H), 1.59-1.46 (m, 1H), 1.25-1.11 (m, 2H).

### Example 45. Synthesis of N-(2-chlorophenyl)-4-((2-((4-(2-(4-((1-(3-((2,6-dioxopiperidin-3-yl)(methyl)amino)phenyl)piperidin-4-yl)methyl)piperazin-1-yl)-2-oxoethyl)phenyl)amino)-fluoropyrimidin-4-yl)amino)benzamide (Compound 45)

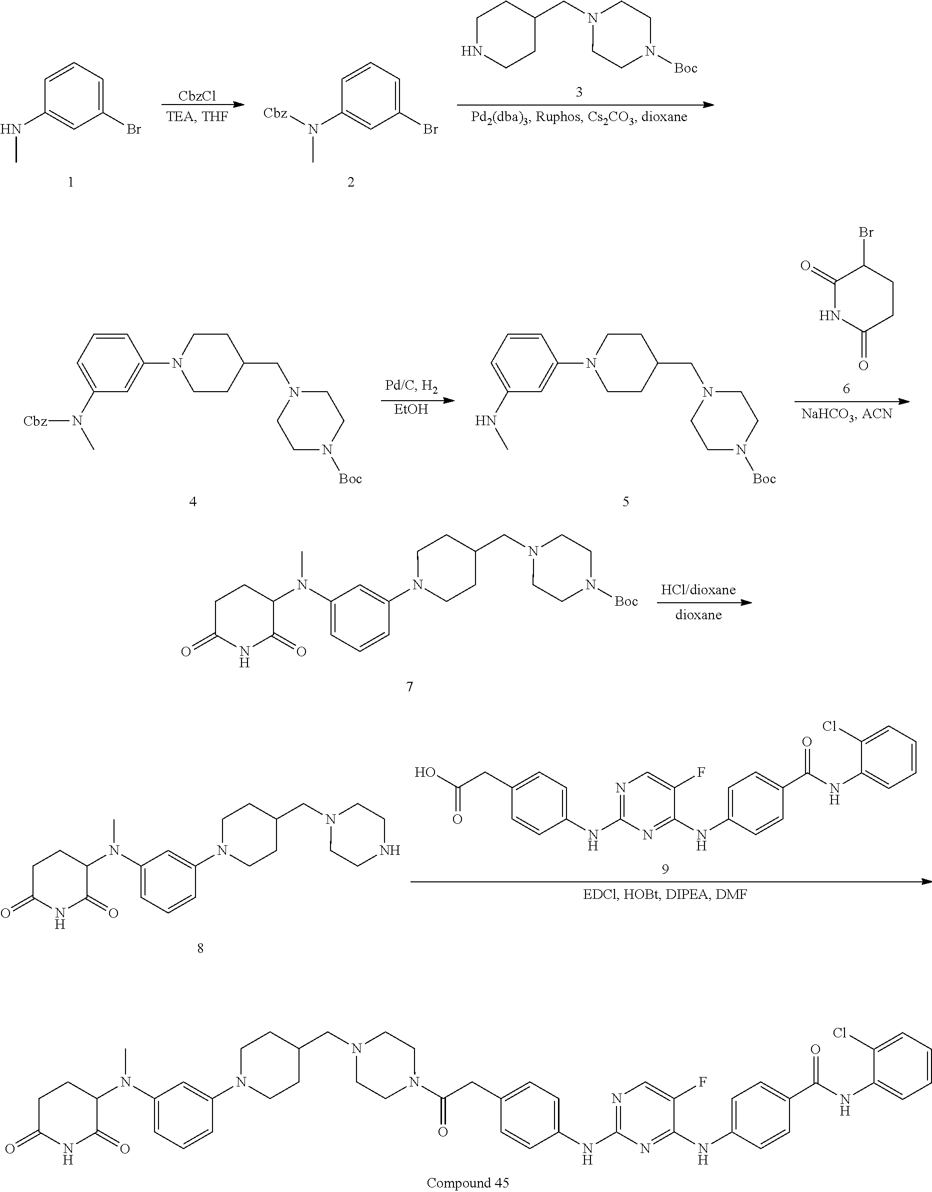

### Step 1. Synthesis of benzyl (3-bromophenyl)(methyl)carbamate (2)

To a solution of 3-bromo-N-methylaniline (1.88 g, 10.10 mmol) in THF (18 mL) was added CbzCl (2.07 g, 12.13 mmol) and TEA (3.07 g, 30.31 mmol, 4.22 mL). The mixture was stirred at 50° C. for 16 hr. LCMS showed 53% of 3-bromo-N-methylaniline remained and 34% of desired mass. Another portion of CbzCl (861.91 mg, 5.05 mmol, 721.26 μL) and TEA (511.25 mg, 5.05 mmol, 703.23 L) were added and the resulting mixture was stirred at 50° C. for another 16 hr. LCMS showed 21% of 3-bromo-N-methyl-aniline remained and 24% of desired mass. The reaction mixture was poured into H₂O (50 mL) and extracted with EtOAc (30 mL×3). The combined organic layers were dried over Na₂SO₄, filtered and concentrated under reduced pressure to give a residue. The residue was purified by flash silica gel chromatography (ISCO®; 40 g SepaFlash® Silica Flash Column, Eluent of 0-4% EtOAc/Petroleum ether, gradient @ 80 mL/min) to afford benzyl (3-bromophenyl)(methyl)carbamate (1.4 g, 4.29 mmol, 42.41% yield, 98% purity) as a colorless oil. MS(M+H)⁺=320.9.

### Step 2. Synthesis of tert-butyl 4-((1-(3-(((benzyloxy)carbonyl)(methyl)amino)phenyl)piperidin-4-yl)methyl)piperazine-1-carboxylate (4)

A mixture of benzyl (3-bromophenyl)(methyl)carbamate (2.33 g, 7.28 mmol), tert-butyl 4-(piperidin-4-ylmethyl)piperazine-1-carboxylate (2.75 g, 5.82 mmol), Cs₂CO₃ (11.86 g, 36.39 mmol), RuPhos (679.15 mg, 1.46 mmol) and Pd₂(dba)₃ (666.38 mg, 727.71 μmol) in dioxane (25 mL) was degassed and purged with N₂ for 3 times, and then the mixture was stirred at 100° C. for 16 hrs under N₂ atmosphere. LCMS showed benzyl (3-bromophenyl)(methyl)carbamate was consumed completely and desired mass. The mixture was filtered and the filtrate was concentrated under reduced pressure to give a residue, which was purified by flash silica gel chromatography (ISCO®; 40 g SepaFlash® Silica Flash Column, Eluent of 0-35% EtOAc/Petroleum ether, gradient @ 60 mL/min) to afford tert-butyl 4-((1-(3-(((benzyloxy)carbonyl)(methyl)amino)phenyl)piperidin-4-yl)methyl)piperazine-1-carboxylate (1.02 g, 1.95 mmol, 26.82% yield) as a brown oil. MS(M+H)⁺=523.3.

### Step 3. Synthesis of tert-butyl 4-((1-(3-(methylamino)phenyl)piperidin-4-yl)methyl)piperazine-1-carboxylate (5)

A solution of tert-butyl 4-((1-(3-(((benzyloxy)carbonyl)(methyl)amino)phenyl)piperidin-4-yl)methyl)piperazine-1-carboxylate (1.02 g, 1.95 mmol) in EtOH (20 mL) saturated with Pd/C (300 mg, 10% purity). The suspension was degassed and purged with H₂ for 3 times. The mixture was stirred under H₂ (15 Psi) at 25° C. for 16 hr. LCMS showed tert-butyl 4-((1-(3-(((benzyloxy)carbonyl)(methyl)amino)phenyl)piperidin-4-yl)methyl)piperazine-1-carboxylate was consumed completely and desired mass. The mixture was filtered and the filtrate was concentrated under reduced pressure to afford tert-butyl 4-((1-(3-(methylamino)phenyl)piperidin-4-yl)methyl)piperazine-1-carboxylate (0.778 g, crude) as a brown oil. The crude product was used for the next step directly. MS(M+H)⁺=389.2.

### Step 4. Synthesis of tert-butyl 4-((1-(3-((2,6-dioxopiperidin-3-yl)(methyl)amino)phenyl)piperidin-4-yl)methyl)piperazine-1-carboxylate (7)

To a solution of tert-butyl 4-((1-(3-(methylamino)phenyl)piperidin-4-yl)methyl)piperazine-1-carboxylate (0.35 g, 900.80 mol) and 3-bromopiperidine-2,6-dione (432.41 mg, 2.25 mmol) in ACN (1.5 mL) was added NaHCO₃ (438.90 mg, 5.22 mmol, 203.29 μL). The mixture was stirred at 80° C. for 14 hr. LCMS showed tert-butyl 4-((1-(3-(methylamino)phenyl)piperidin-4-yl)methyl)piperazine-1-carboxylate was consumed completely and desired mass. The reaction mixture was poured into H₂O (20 mL) and extracted with EtOAc (10 mL×3). The combined organic layers were dried over Na₂SO₄, filtered and concentrated under reduced pressure to give a residue, which was purified by flash silica gel chromatography (ISCO®; 20 g SepaFlash® Silica Flash Column, Eluent of 0-40% EtOAc/Petroleum ether, gradient @ 50 mL/min). The crude product was further purified by prep-TLC (SiO₂, DCM:MeOH=10:1) to afford tert-butyl 4-((1-(3-((2,6-dioxopiperidin-3-yl)(methyl)amino)phenyl)piperidin-4-yl)methyl)piperazine-1-carboxylate (0.12 g, 240.17 μmol, 26.66% yield) as colorless oil. MS(M+H)⁺=500.4.

### Step 5. Synthesis of 3-(methyl(3-(4-(piperazin-1-ylmethyl)piperidin-1-yl)phenyl)amino)piperidine-2,6-dione (8)

To a solution of tert-butyl 4-((1-(3-((2,6-dioxopiperidin-3-yl)(methyl)amino)phenyl)piperidin-4-yl)methyl)piperazine-1-carboxylate (100 mg, 200.14 mol) in dioxane (1 mL) was added HCl/dioxane (4 M, 2 mL). The mixture was stirred at 20° C. for 0.5 hr. LCMS showed the starting material was consumed completely and the desired mass. The reaction mixture was concentrated under reduced pressure to afford 3-(methyl(3-(4-(piperazin-1-ylmethyl)piperidin-1-yl)phenyl)amino)piperidine-2,6-dione (87 mg, 199.55 μmol, 99.70% yield, HCl) as a white solid. MS(M+H)⁺=400.2.

### Step 6. Synthesis of N-(2-chlorophenyl)-4-((2-((4-(2-(4-((1-(3-((2,6-dioxopiperidin-3-yl)(methyl)amino)phenyl)piperidin-4-yl)methyl)piperazin-1-yl)-2-oxoethyl)phenyl)amino)-5-fluoropyrimidin-4-yl)amino)benzamide (Compound 45)

To a solution of 3-(methyl(3-(4-(piperazin-1-ylmethyl)piperidin-1-yl)phenyl)amino)piperidine-2,6-dione (87 mg, 199.55 μmol, HCl), 2-(4-((4-((4-((2-chlorophenyl)carbamoyl)phenyl)amino)-5-fluoropyrimidin-2-yl)amino)phenyl)acetic acid (98.16 mg, 199.55 mol), EDCI (57.38 mg, 299.32 mol) and HOBt (26.96 mg, 199.55 μmol) in DMF (1 mL) was added DIPEA (128.95 mg, 997.73 mol, 173.79 μL). The mixture was stirred at 25° C. for 2 hr. LCMS showed 3-(methyl(3-(4-(piperazin-1-ylmethyl)piperidin-1-yl)phenyl)amino)piperidine-2,6-dion e was consumed completely and desired mass. The reaction mixture was poured into water (20 mL) and extracted with EtOAc (10 mL×3). The combined organic layers were washed with brine, dried over Na₂SO₄, filtered and concentrated under reduced pressure to give a residue. The residue was purified by flash silica gel chromatography (ISCO®; 20 g SepaFlash® Silica Flash Column, Eluent of 0-10% MeOH/DCM @ 50 mL/min), followed by prep-HPLC (column: Waters Xbridge 150*25 mm*5 um; mobile phase: [water (NH₄HCO₃)-ACN]; gradient: 44%-74% B over 10 min) to afford N-(2-chlorophenyl)-4-((2-((4-(2-(4-((1-(3-((2,6-dioxopiperidin-3-yl)(methyl)amino)phen yl)piperidin-4-yl)methyl)piperazin-1-yl)-2-oxoethyl)phenyl)amino)-5-fluoropyrimidin-4-yl)amino)benzamide (24.7 mg, 27.15 μmol, 13.61% yield, 96% purity) as a white solid. MS (M+H)⁺=873.5.

¹H NMR (400 MHz, DMSO-d₆) δ=10.75 (s, 1H), 9.88 (s, 1H), 9.65 (s, 1H), 9.28 (s, 1H), 8.18 (d, J=3.5 Hz, 1H), 8.09-8.00 (m, 2H), 8.00-7.93 (m, 2H), 7.70-7.63 (m, 1H), 7.60 (d, J=8.6 Hz, 2H), 7.58-7.53 (m, 1H), 7.43-7.35 (m, 1H), 7.33-7.25 (m, 1H), 7.12 (d, J=8.7 Hz, 2H), 6.96 (t, J=8.1 Hz, 1H), 6.33-6.21 (m, 3H), 4.92-4.78 (m, 1H), 3.69-3.54 (m, 4H), 3.50-3.40 (m, 4H), 2.92-2.77 (m, 1H), 2.69 (s, 3H), 2.64-2.52 (m, 3H), 2.35-2.17 (m, 5H), 2.12-2.08 (m, 2H), 1.91-1.79 (m, 1H), 1.75-1.68 (m, 2H), 1.66-1.51 (m, 1H), 1.24-1.08 (m, 2H).

### Example 46. Synthesis of N-(2-chlorophenyl)-4-((2-((4-(2-(4-((1-(4-((2,6-dioxopiperidin-3-yl)carbamoyl)phenyl)piperidin-4-yl)methyl)piperazin-1-yl)-2-oxoethyl)phenyl)amino)-5-fluoropyrimidin-4-yl)amino)benzamide (Compound 46)

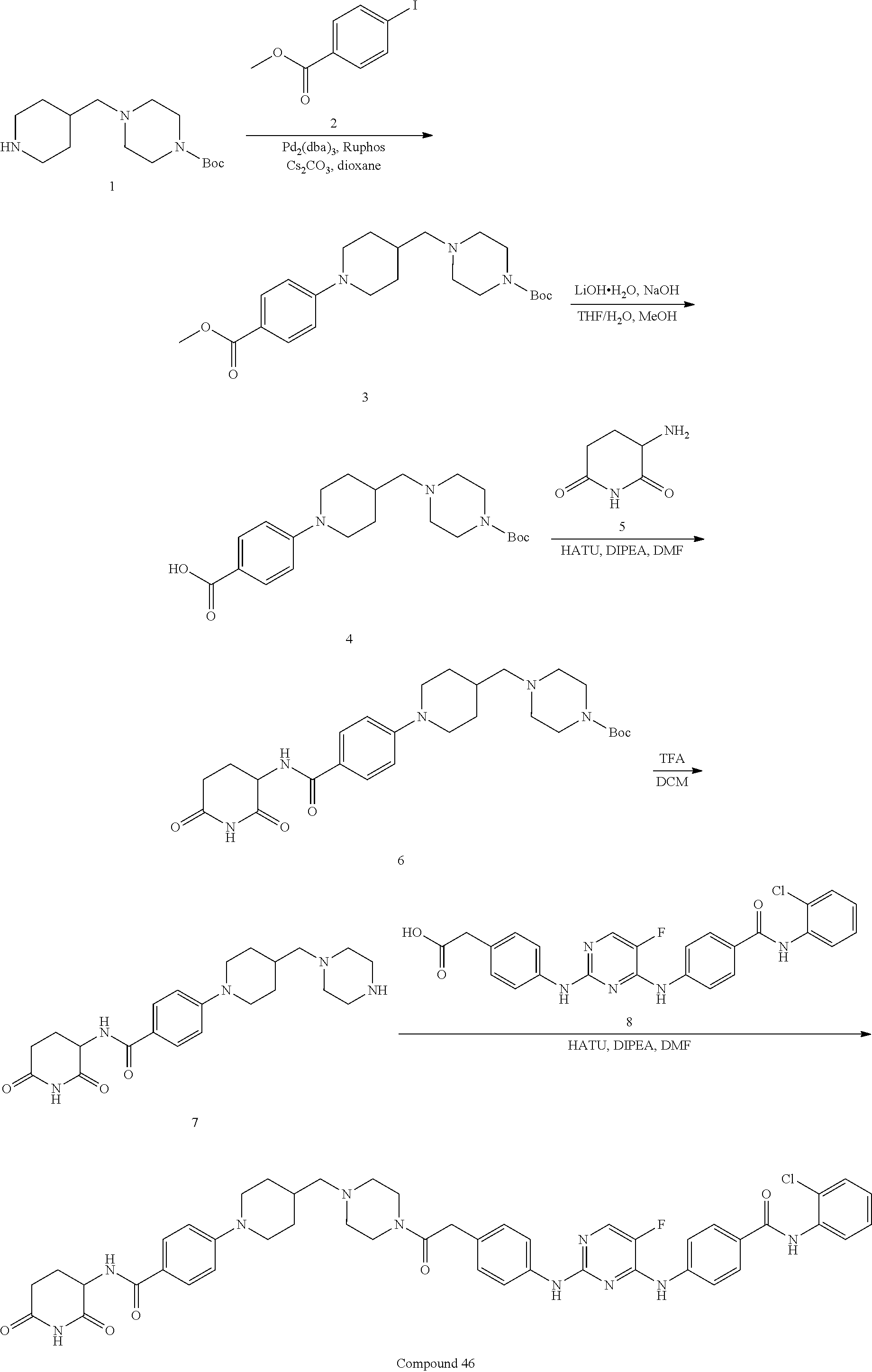

### Step 1. Synthesis of tert-butyl 4-((1-(4-(methoxycarbonyl)phenyl)piperidin-4-yl)methyl)piperazine-1-carboxylate (3)

A mixture of methyl 4-iodobenzoate (1 g, 3.82 mmol), tert-butyl 4-(piperidin-4-ylmethyl)piperazine-1-carboxylate (1.57 g, 4.58 mmol, HOAC), Pd₂ (dba)₃ (349.45 mg, 381.62 mol), Cs₂CO₃ (6.22 g, 19.08 mmol) and RuPhos (356.15 mg, 763.23 mol) in dioxane (15 mL) was degassed and purged with N₂ for 3 times, and then stirred at 100° C. for 16 hrs under N₂ atmosphere. LCMS showed tert-butyl 4-(piperidin-4-ylmethyl)piperazine-1-carboxylate was consumed completely and desired mass. The reaction mixture was filtered and concentrated under reduced pressure. The residue was purified by flash silica gel chromatography (ISCO®; 20 g SepaFlash® Silica Flash Column, Eluent of 0~20% EtOAc/Petroleum ether gradient @50 mL/min) to afford tert-butyl 4-((1-(4-(methoxycarbonyl)phenyl)piperidin-4-yl)methyl)piperazine-1-carboxylate (1.13 g, 2.07 mmol, 54.31% yield, 76.58% purity) as a yellow solid. MS(M+H)⁺=418.2.

### Step 2. Synthesis of 4-(4-((4-(tert-butoxycarbonyl)piperazin-1-yl)methyl)piperidin-1-yl)benzoic acid (4)

To a solution of tert-butyl 4-((1-(4-(methoxycarbonyl)phenyl)piperidin-4-yl)methyl)piperazine-1-carboxylate (1.13 g, 2.07 mmol, 76.58% purity) in THF (8 mL) was added a solution of LiOH·H₂O (521.77 mg, 12.44 mmol) in H₂O (2 mL). The reaction mixture was stirred at 40° C. for 16 hr. LCMS showed 80% of tert-butyl 4-((1-(4-(methoxycarbonyl)phenyl)piperidin-4-yl)methyl)piperazine-1-carboxylate remained and 5.9% peak of desired mass. Another portion of LiOH·H₂O (200 mg) was added and the mixture was stirred at 50° C. for 16 hr. LCMS showed 76.38% peak of tert-butyl 4-((1-(4-(methoxycarbonyl)phenyl)piperidin-4-yl)methyl)piperazine-1-carboxylate remained. To the reaction mixture was added MeOH (5 mL) and NaOH (248.68 mg, 6.22 mmol). The resulting mixture was stirred at 50° C. for 9 hr. LCMS showed tert-butyl 4-((1-(4-(methoxycarbonyl)phenyl)piperidin-4-yl)methyl)piperazine-1-carboxylate was consumed completely. The reaction mixture was neutralized with 1N HCl solution and then extracted with EtOAc. The combined organic layers were dried over Na₂SO₄, filtered and concentrated under reduced pressure to afford 4-(4-((4-(tert-butoxycarbonyl)piperazin-1-yl)methyl)piperidin-1-yl)benzoic acid (0.893, 2.06 mmol, 99.31% yield, 93% purity) as a yellow solid. MS(M+H)⁺=404.2.

### Step 3. Synthesis of tert-butyl 4-((1-(4-((2,6-dioxopiperidin-3-yl)carbamoyl)phenyl)piperidin-4-yl)methyl)piperazine-1-carboxylate (6)

To a solution of 4-(4-((4-(tert-butoxycarbonyl)piperazin-1-yl)methyl)piperidin-1-yl)benzoic acid (0.378 g, 936.77 mol) and HATU (534.28 mg, 1.41 mmol) in DMF (3 mL) was added DIPEA (605.36 mg, 4.68 mmol, 815.84 μL) and stirred at 25° C. for 0.5 hr. Then 3-aminopiperidine-2,6-dione (123.35 mg, 749.42 μmol, HCl) was added and the resulting mixture and stirred at 25° C. for 16 hr. LCMS showed the starting material was consumed completely and the desired mass. The reaction mixture was poured into water and extracted with EtOAc. The combined organic layers were washed with brine, dried over Na₂SO₄, filtered and concentrated. The residue was purified by flash silica gel chromatography (ISCO®; 20 g SepaFlash® Silica Flash Column, Eluent of 0-100% EtOAc/Petroleum ether gradient @ 50 mL/min) to afford tert-butyl 4-((1-(4-((2,6-dioxopiperidin-3-yl)carbamoyl)phenyl)piperidin-4-yl)methyl)piperazine-1-carboxylate (0.435 g, 770.69 μmol, 82.27% yield, 91% purity) as a yellow solid. MS(M+H)⁺=514.2.

### Step 4. Synthesis of N-(2,6-dioxopiperidin-3-yl)-4-(4-(piperazin-1-ylmethyl)piperidin-1-yl)benzamide (7)

To a solution of tert-butyl 4-((1-(4-((2,6-dioxopiperidin-3-yl)carbamoyl)phenyl)piperidin-4-yl)methyl)piperazine-1-carboxylate (0.2 g, 389.39 mol) in DCM (3 mL) was added TFA (44.40 mg, 389.39 μmol, 1 mL). The mixture was stirred at 20° C. for 2 hr. LCMS showed the starting material was consumed completely. The reaction mixture was concentrated under reduced pressure to afford N-(2,6-dioxopiperidin-3-yl)-4-(4-(piperazin-1-ylmethyl)piperidin-1-yl)benzamide (0.2 g, 379.12 μmol, 97.36% yield, TFA) as a green solid. The crude product was used for the next step without further purification. MS(M+H)⁺=414.5.

### Step 5. Synthesis of N-(2-chlorophenyl)-4-((2-((4-(2-(4-((1-(4-((2,6-dioxopiperidin-3-yl)carbamoyl)phenyl)piperidin-4-yl)methyl)piperazin-1-yl)-2-oxoethyl)phenyl)amino)-5-fluoropyrimidin-4-yl) amino)benzamide (Compound 46)

To a solution of 2-(4-((4-((4-((2-chlorophenyl)carbamoyl)phenyl)amino)-5-fluoropyrimidin-2-yl)amino)phenyl)acetic acid (228.45 mg, 371.54 μmol, 80% purity) and HATU (211.91 mg, 557.31 mol) in DMF (2 mL) was added DIPEA (288.11 mg, 2.23 mmol, 388.29 μL). Then N-(2,6-dioxopiperidin-3-yl)-4-(4-(piperazin-1-ylmethyl)piperidin-1-yl)benzamide (0.2 g, 371.54 μmol, 98% purity, TFA) was added and the resulting mixture was stirred at 20° C. for 16 hr. LCMS showed N-(2,6-dioxopiperidin-3-yl)-4-(4-(piperazin-1-ylmethyl)piperidin-1-yl)benzamide was consumed completely and desired mass. The reaction mixture was poured into water and extracted with EtOAc. The combined organic layers were washed with brine, dried over Na₂SO₄, filtered and concentrated. The residue was purified by flash silica gel chromatography (ISCO®; 12 g SepaFlash® Silica Flash Column, Eluent of 0-100% EtOAc/petroleum ether gradient @ 50 mL/min). The crude product was purified by prep-TLC (SiO₂, DCM:MeOH=10:1) to afford N-(2-chlorophenyl)-4-((2-((4-(2-(4-((1-(4-((2,6-dioxopiperidin-3-yl)carbamoyl)phenyl)piperidin-4-yl)methyl)piperazin-1-yl)-2-oxoethyl)phenyl)amino)-5-fluoropyrimidin-4-yl) amino)benzamide (17.8 mg, 20.06 μmol, 5.40% yield) as a white solid. MS(M+H)⁺=887.3.

¹H NMR (400 MHz, DMSO-d₆) δ=10.82 (s, 1H), 9.89 (s, 1H), 9.66 (s, 1H), 9.29 (s, 1H), 8.48-8.40 (m, 1H), 8.18 (d, J=3.5 Hz, 1H), 8.08-7.93 (m, 4H), 7.72 (br d, J=8.8 Hz, 2H), 7.68-7.54 (m, 4H), 7.42-7.36 (m, 1H), 7.32-7.26 (m, 1H), 7.12 (d, J=8.6 Hz, 2H), 6.93 (br d, J=8.4 Hz, 2H), 4.79-4.67 (m, 1H), 3.93-3.83 (m, 2H), 3.64 (s, 2H), 3.50-3.38 (m, 4H), 2.85-2.68 (m, 3H), 2.57-2.52 (m, 2H), 2.30-2.16 (m, 4H), 2.13-2.08 (m, 2H), 1.99-1.89 (m, 1H), 1.80-1.58 (m, 3H), 1.19-1.04 (m, 2H).

### Example 47. Synthesis of 3-(4-((4-(2-(4-((4-((4-((2-chlorophenyl)carbamoyl)phenyl)amino)-5-fluoropyrimidin-2-yl)amino)phenyl)acetyl)piperazin-1-yl)methyl)piperidin-1-yl)-N-(2,6-dioxopiperidin-3-yl)benzamide (Compound 47)

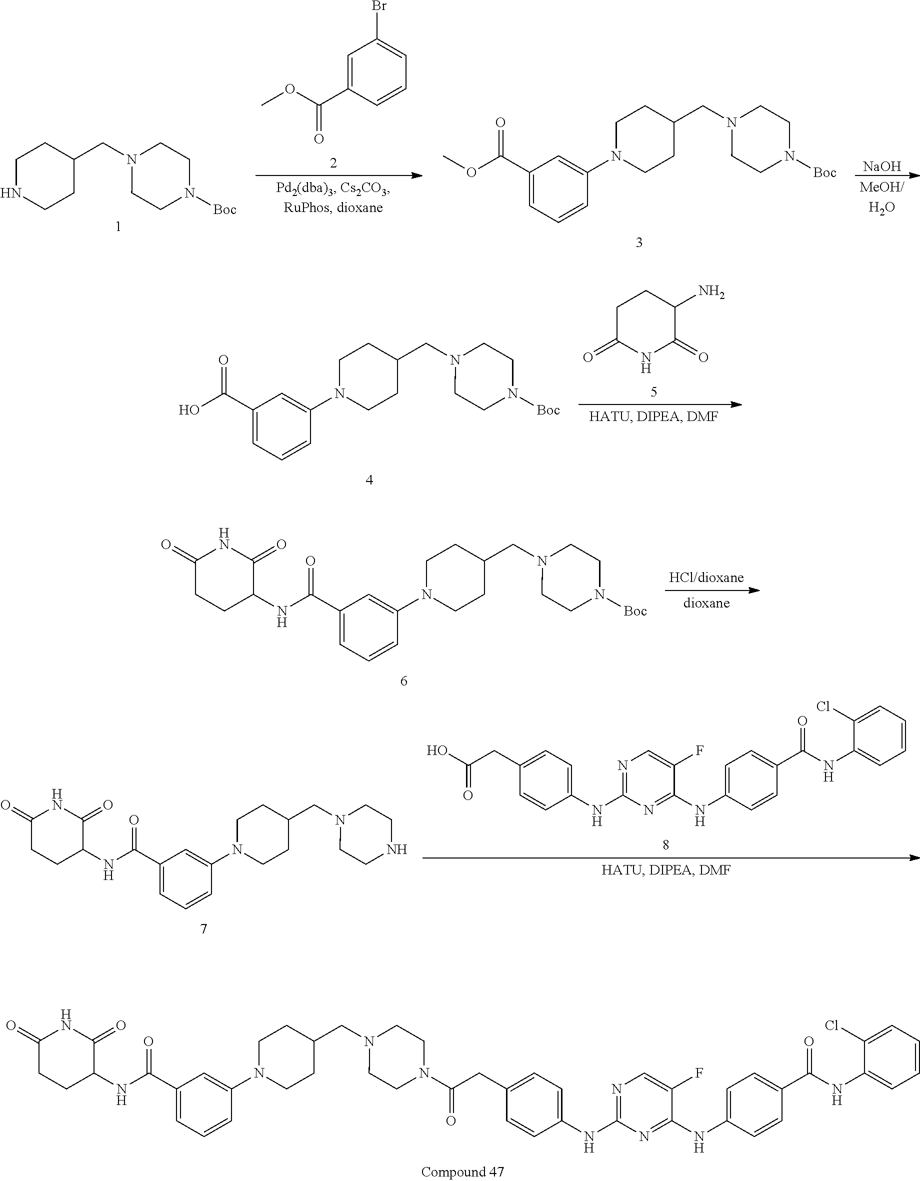

### Step 1. Synthesis of tert-butyl 4-((1-(3-(methoxycarbonyl)phenyl)piperidin-4-yl)methyl)piperazine-1-carboxylate (3)

A mixture of tert-butyl 4-(piperidin-4-ylmethyl)piperazine-1-carboxylate (1.58 g, 4.60 mmol), methyl 3-bromobenzoate (1 g, 4.65 mmol), Pd₂(dba)₃ (425.83 mg, 465.02 mol), Cs₂CO₃ (7.58 g, 23.25 mmol) and RuPhos (433.99 mg, 930.04 mol) in dioxane (15 mL) was degassed and purged with N₂ for 3 times, and then the mixture was stirred at 100° C. for 16 hr under N₂ atmosphere. LCMS showed tert-butyl 4-(piperidin-4-ylmethyl)piperazine-1-carboxylate was consumed completely and desired mass. The reaction mixture was filtered and concentrated under reduced pressure to give a residue, which was purified by flash silica gel chromatography (ISCO®; 20 g SepaFlash® Silica Flash Column, Eluent of 0~50% EtOAc/Petroleum ether, gradient @ 50 mL/min) to afford tert-butyl 4-((1-(3-(methoxycarbonyl)phenyl)piperidin-4-yl)methyl)piperazine-1-carboxylate (0.75 g, 1.01 mmol, 21.63% yield, 56% purity) as a yellow solid. MS(M+H)⁺=418.2.

### Step 2. Synthesis of 3-(4-((4-(tert-butoxycarbonyl)piperazin-1-yl)methyl)piperidin-1-yl)benzoic acid (4)

To a solution of tert-butyl 4-((1-(3-(methoxycarbonyl)phenyl)piperidin-4-yl)methyl)piperazine-1-carboxylate (0.75 g, 1.80 mmol) in MeOH (10 mL) was added a solution of NaOH (215.53 mg, 5.39 mmol) in H₂O (1 mL). The mixture was stirred at 25° C. for 12 hr. LCMS showed tert-butyl 4-((1-(3-(methoxycarbonyl)phenyl)piperidin-4-yl)methyl)piperazine-1-carboxylate was consumed completely and desired mass. The reaction mixture was concentrated to remove the MeOH. The residue was diluted with water (20 mL) and extracted with MTBE to remove the impurities. The residual aqueous solution was acidified with 1N HCl solution to pH=4 and extracted with EtOAc (10 mL×3). The combined organic layers were dried over Na₂SO₄, filtered and concentrated under reduced pressure to afford 3-(4-((4-(tert-butoxycarbonyl)piperazin-1-yl)methyl)piperidin-1-yl)benzoic acid (0.65 g, 1.61 mmol, 89.68% yield) as a yellow oil. The crude product was used for the next step directly. MS(M+H)⁺=404.4.

### Step 3. Synthesis of tert-butyl 4-((1-(3-((2,6-dioxopiperidin-3-yl)carbamoyl)phenyl)piperidin-4-yl)methyl)piperazine-1-carboxylate (6)

A solution of 3-(4-((4-(tert-butoxycarbonyl)piperazin-1-yl)methyl)piperidin-1-yl)benzoic acid (0.65 g, 1.61 mmol), HATU (918.74 mg, 2.42 mmol) and DIPEA (1.04 g, 8.05 mmol, 1.40 mL) in DMF (1 mL) was stirred at 25° C. for 0.5 hr. And then 3-aminopiperidine-2, 6-dione (212.10 mg, 1.29 mmol, HCl) was added and the resulting mixture was stirred at 25° C. for 16 hr. LCMS showed 3-(4-((4-(tert-butoxycarbonyl)piperazin-1-yl)methyl)piperidin-1-yl)benzoic acid was consumed completely and desired mass. The reaction mixture was poured into water (15 mL) extracted with EtOAc (10 mL×3). The combined organic layers were washed with brine, dried over Na₂SO₄, filtered and concentrated under reduced pressure to give a residue, which was purified by flash silica gel chromatography (ISCO®; 20 g SepaFlash® Silica Flash Column, Eluent of 0-100% EtOAc/Petroleum ether, gradient @ 50 mL/min) to afford tert-butyl 4-((1-(3-((2,6-dioxopiperidin-3-yl)carbamoyl)phenyl)piperidin-4-yl)methyl)piperazine-1-carboxylate (0.508 g, 969.26 μmol, 60.17% yield, 98% purity) as a brown solid. MS (M+H)⁺=514.3.

### Step 4. Synthesis of N-(2,6-dioxopiperidin-3-yl)-3-(4-(piperazin-1-ylmethyl)piperidin-1-yl)benzamide (7)

A solution of tert-butyl 4-((1-(3-((2,6-dioxopiperidin-3-yl)carbamoyl)phenyl)piperidin-4-yl)methyl)piperazine-1-carboxylate (100 mg, 200.14 mol) in dioxane (1 mL) was added HCl/dioxane (4 M, 2 mL). The mixture was stirred at 20° C. for 0.5 hr. LCMS showed the starting material was consumed completely and the desired mass. The reaction mixture was concentrated under reduced pressure to afford N-(2,6-dioxopiperidin-3-yl)-3-(4-(piperazin-1-ylmethyl)piperidin-1-yl)benzamide (0.17 g, 277.07 μmol, 90.46% yield, 92.50% purity, TFA) as a brown oil. The crude product was used for the next step directly. MS(M+H)⁺=414.5.

### Step 5. Synthesis of 3-(4-((4-(2-(4-((4-((4-((2-chlorophenyl)carbamoyl)phenyl)amino)-5-fluoropyrimidin-2-yl)amino)phenyl)acetyl)piperazin-1-yl)methyl)piperidin-1-yl)-N-(2,6-dioxopiperidin-3-yl)benzamide (Compound 47)

A mixture of 2-(4-((4-((4-((2-chlorophenyl)carbamoyl)phenyl)amino)-5-fluoropyrimidin-2-yl)amino)phenyl)acetic acid (240.50 mg, 488.92 mol), HATU (278.85 mg, 733.38 mol) and DIPEA (315.95 mg, 2.44 mmol, 425.80 μL) in DMF (2 mL) was stirred at 20° C. for 0.5 hr. And then N-(2,6-dioxopiperidin-3-yl)-3-(4-(piperazin-1-ylmethyl)piperidin-1-yl)benzamide (0.22 g, 488.92 μmol, HCl) was added. The resulting mixture was stirred at 20° C. for 16 hr. LCMS showed N-(2,6-dioxopiperidin-3-yl)-3-(4-(piperazin-1-ylmethyl)piperidin-1-yl)benzamide was consumed completely and desired mass. The reaction mixture was poured into water (10 mL) and extracted with EtOAc (10 mL×3). The combined organic layers were washed with brine, dried over Na₂SO₄, filtered and concentrated under reduced pressure to give a residue. The residue was purified by flash silica gel chromatography (ISCO®; 12 g SepaFlash® Silica Flash Column, Eluent of 0-10% MeOH/EtOAc @50 mL/min), followed by prep-HPLC (column: Waters Xbridge 150*25 mm 10 um; mobile phase: [water (NH₄HCO₃)-ACN]; B %: 40%-70%, 10 min) to afford 3-(4-((4-(2-(4-((4-((4-((2-chlorophenyl)carbamoyl)phenyl)amino)-5-fluoropyrimidin-2-yl)amino)phenyl)acetyl)piperazin-1-yl)methyl)piperidin-1-yl)-N-(2,6-dioxopiperidin-3-yl)benzamide (13.7 mg, 14.20 μmol, 2.91% yield, 92% purity) as a white solid. MS(M+H)⁺=887.4.

¹H NMR (400 MHz, DMSO-d₆) δ=10.85 (s, 1H), 9.88 (s, 1H), 9.65 (s, 1H), 9.28 (s, 1H), 8.66 (d, J=8.6 Hz, 1H), 8.18 (d, J=3.7 Hz, 1H), 8.06-7.93 (m, 4H), 7.69-7.53 (m, 4H), 7.42-7.34 (m, 2H), 7.32-7.21 (m, 3H), 7.16-7.05 (m, 3H), 4.81-4.71 (m, 1H), 3.71 (br d, J=11.6 Hz, 2H), 3.64 (s, 2H), 3.50-3.40 (m, 4H), 2.86-2.74 (m, 1H), 2.72-2.61 (m, 2H), 2.57-2.52 (m, 1H), 2.31-2.19 (m, 4H), 2.17-2.07 (m, 3H), 2.01-1.92 (m, 1H), 1.82-1.71 (m, 2H), 1.71-1.59 (m, 1H), 1.24-1.10 (m, 2H).

### Example 48. Synthesis of N-(2-chlorophenyl)-4-((2-((4-((4-(2-(4-(4-(2,6-dioxopiperidin-3-yl)-2-fluorophenyl)piperazin-1-yl)ethyl)piperidin-1-yl)carbamoyl)phenyl)amino)-5-fluoropyrimidin-4-yl)amino)benzamide (Compound 48)

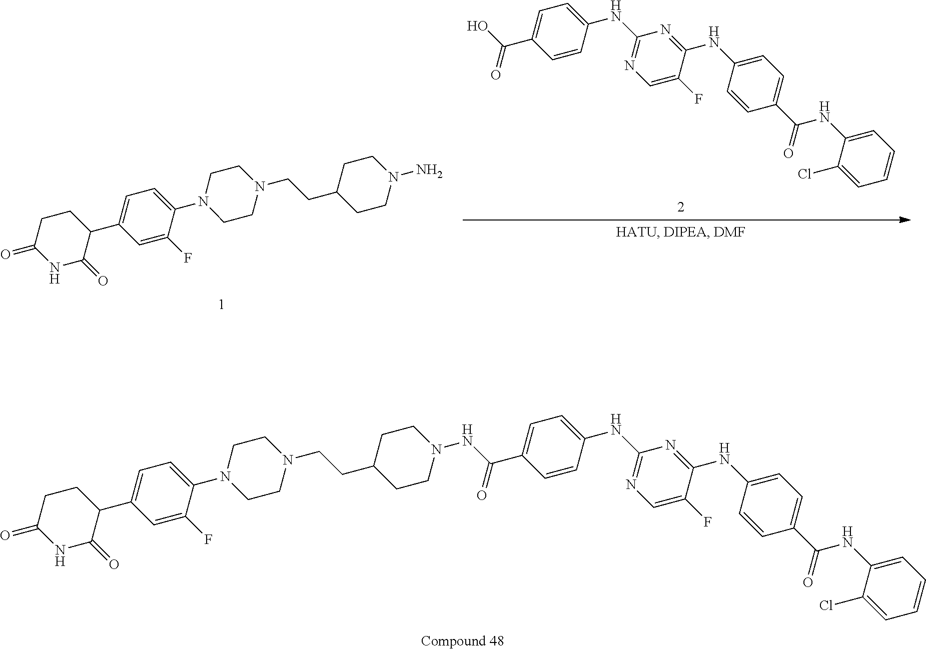 [SMILES: NN1CCC(CCN2CCN(c3ccc(C4CCC(=O)NC4=O)cc3F)CC2)CC1 ; O=C(O)c1ccc(Nc2ncc(F)c(Nc3ccc(C(=O)Nc4ccccc4Cl)cc3)n2)cc1 ; O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)c(F)c2)C(=O)N1]

### Step 1. Synthesis of N-(2-chlorophenyl)-4-((2-((4-((4-(2-(4-(4-(2,6-dioxopiperidin-3-yl)-2-fluorophenyl)piperazin-1-ylethyl)piperidin-1-yl)carbamoyl)phenyl)amino)-5-fluoropyrimidin-4-yl)amino)benzamide (Compound 48)

To a solution of 4-((4-((4-((2-chlorophenyl)carbamoyl)phenyl)amino)-5-fluoropyrimidin-2-yl) amino)benzoic acid (80 mg, 167.41 mol) and HATU (76.38 mg, 200.89 mol) in DMF (1 mL) was added DIPEA (148.40 mg, 1.15 mmol, 0.2 mL) and the mixture was stirred at 20° C. for 10 min. Then 3-(4-(4-(2-(1-aminopiperidin-4-yl)ethyl)piperazin-1-yl)-3-fluorophenyl)piperidine-2,6-dione (90 mg, 169.32 μmol, TFA) in DMF (1 mL) was added and the mixture was stirred at 20° C. for 1 h. LCMS showed 57% peak with the desired mass. The mixture was diluted with water (5 mL) and then filtered. The filter cake was washed with water (5 mL), collected and dried to afford the crude product. The crude was purified by prep-HPLC (column: Phenomenex Luna C18 150*25 mm*10 um; mobile phase: [water (FA)-ACN]; gradient: 25%-55% B over 9 min) and the eluent was adjusted the pH=7 with saturated NaHCO₃ and the mixture was extracted with EtOAc:DCM=1:1 (20 mL×2). The combined organic layer was concentrated under reduced pressure. The product was diluted with water (0.5 mL) and stirred at 20° C. for 0.5 h. The mixture was filtered and the filter cake was washed with water (0.5 mL). The filter cake was diluted with MeCN (3 mL) and deionized water (20 mL) and then the mixture was lyophilized. The product was diluted with EtOAc:MeOH=10:1 (0.5 mL) and stirred at 20° C. for 0.5 h. The mixture was filtered and the filter cake was washed with EtOAc:MeOH=10:1 (1 mL) and water (0.5 mL). The filter cake was diluted with MeCN (3 mL) and deionized water (20 mL) and then lyophilized to afford N-(2-chlorophenyl)-4-((2-((4-((4-(2-(4-(4-(2,6-dioxopiperidin-3-yl)-2-fluorophenyl)piperazin-1-yl)ethyl)piperidin-1-yl)carbamoyl)phenyl)amino)-5-fluoropyrimidin-4-yl)amino)benzamide (8.6 mg, 9.61 μmol, 5.74% yield, 98% purity) as a white solid. MS(M+H)⁺=877.4.

¹H NMR (400 MHz, DMSO-d₆) δ=10.85-10.75 (m, 1H), 9.92 (br s, 1H), 9.74 (br s, 1H), 9.60 (br s, 1H), 9.12 (br s, 1H), 8.24 (br s, 1H), 8.08-7.93 (m, 4H), 7.84-7.61 (m, 5H), 7.60-7.50 (m, 1H), 7.42-7.35 (m, 1H), 7.33-7.24 (m, 1H), 7.08-6.87 (m, 3H), 3.84-3.75 (m, 1H), 3.31-3.28 (m, 4H), 3.09-2.90 (m, 6H), 2.73-2.65 (m, 3H), 2.37-2.30 (m, 2H), 2.24-2.14 (m, 1H), 2.06-1.92 (m, 1H), 1.76-1.62 (m, 2H), 1.48-1.36 (m, 2H), 1.32-1.20 (m, 4H).

### Example 49. Synthesis of N-(2-chlorophenyl)-4-((2-((4-(((1r,4r)-4-(((1-(4-(2,6-dioxopiperidin-3-yl)phenyl)piperidin-4-yl)methyl)(methyl)amino)cyclohexyl)carbamoyl)phenyl)amino)-5-fluoropyrimidin-4-yl)amino)benzamide (trans) (Compound 49)

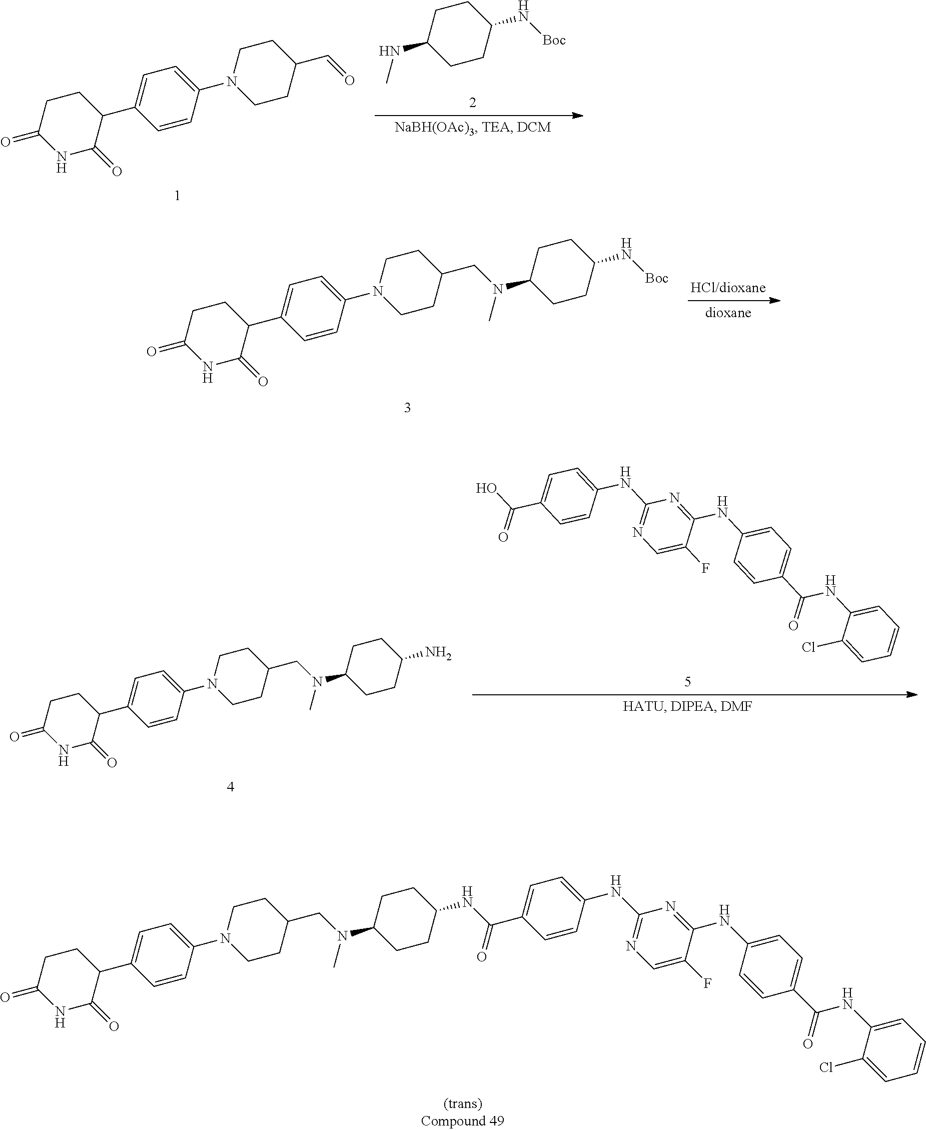

### Step 1. Synthesis of tert-buty ((1r,4r)-4-(((1-(4-(2,6-dioxopiperidin-3-yl)phenyl)piperidin-4-yl)methyl)(methyl)amino)cyclohexyl)carbamate (3)

To a solution of 1-[4-(2,6-dioxo-3-piperidyl)phenyl]piperidine-4-carbaldehyde (100 mg, 332.94 mol) and tert-butyl ((1r,4r)-4-(methylamino)cyclohexyl)carbamate (76.02 mg, 332.94 mol) in DCM (2 mL) was added TEA (101.07 mg, 998.83 μmol, 139.02 L) and 4 Å MS (100 mg, 332.94 mol) at 15° C. After stirring at 15° C. for 30 min, NaBH(OAc)₃ (211.69 mg, 998.83 mol) was added. The resulting mixture was stirred at 15° C. for 16 hr. LCMS showed a peak (59%) with the desired mass. The reaction mixture was diluted with water (15 mL) and extracted with EtOAc (5 mL×4). The combined organic phase was dried over Na₂SO₄, filtered and concentrated under reduced pressure to afford a residue. The residue was purified by flash silica gel chromatography (12 g SepaFlash® Silica Flash Column, Eluent of 0~10% MeOH in EtOAc gradient @ 60 mL/min) to afford tert-butyl ((1r,4r)-4-(((1-(4-(2,6-dioxopiperidin-3-yl)phenyl)piperidin-4-yl)methyl)(methyl)amino)cyclohexyl)carbamate (100 mg, 169.70 μmol, 50.97% yield, 87% purity) as a white solid. MS(M+H)⁺=513.3.

### Step 2. Synthesis of 3-(4-(4-((((1r,4r)-4-aminocyclohexyl)(methyl)amino)methyl)piperidin-1-yl)phenyl)piperidine-2,6-dione (4)

To a solution of tert-butyl ((1r,4r)-4-(((1-(4-(2,6-dioxopiperidin-3-yl)phenyl)piperidin-4-yl)methyl)(methyl)amino)cyclohexyl)carbamate (100 mg, 195.05 mol) in dioxane (1 mL) was added HCl/dioxane (4 M, 1 mL). The mixture was stirred at 15° C. for 2 hr. LCMS showed a main peak with the desired mass. The reaction mixture was concentrated under reduced pressure to afford 3-[4-[4-[[(4-aminocyclohexyl)-methyl-amino]methyl]-1-piperidyl]phenyl]piperidine-2, 6-dione (80 mg, 178.16 μmol, 91.34% yield, 100% purity, HCl) as a light yellow solid. MS(M+H)⁺=413.2.

### Step 3. Synthesis of N-(2-chlorophenyl)-4-((2-((4-(((1r,4r)-4-(((1-(4-(2,6-dioxopiperidin-3-yl)phenyl)piperidin-4-yl)methyl)(methyl)amino)cyclohexyl)carbamoyl)phenyl)amino)-5-fluoropyrimidin-4-yl)amino)benzamide (trans) (Compound 49)

To a solution of 3-(4-(4-((((1r,4r)-4-aminocyclohexyl)(methyl)amino)methyl)piperidin-1-yl)phenyl)piperidine-2,6-dione (60 mg, 133.62 μmol, HCl), 4-[[4-[4-[(2-chlorophenyl)carbamoyl]anilino]-5-fluoro-pyrimidin-2-yl]amino]benzoic acid (63.85 mg, 133.62 mol) and DIPEA (51.81 mg, 400.87 μmol, 69.82 μL) in DMF (1 mL) was added HATU (76.21 mg, 200.43 μmol). The mixture was stirred at 15° C. for 1 hr. LCMS showed a peak (54%) with the desired mass. The reaction mixture was diluted with 10 ml of water and extracted with EtOAc (5 ml×3). The combined organic phase was washed with brine (10 ml×2) and concentrated under reduced pressure. The residue was purified by prep-HPLC (column: Phenomenex Luna C18 150*25 mm*Oum; mobile phase: [water(TFA)-ACN]; gradient: 25%-45% B over 7 min), followed by prep-HPLC (column: Waters Xbridge 150*25 mm*5 um; mobile phase: [water(NH₄HCO₃)-ACN]; gradient: 32%-62% B over 10 min) and lyophilized to afford N-(2-chlorophenyl)-4-((2-((4-(((1r,4r)-4-(((1-(4-(2,6-dioxopiperidin-3-yl)phenyl)piperidin-4-yl)methyl)(methyl)amino)cyclohexyl)carbamoyl)phenyl)amino)-5-fluoropyrimidin-4-yl)amino)benzamide (20.3 mg, 22.80 μmol, 17.07% yield, 98% purity) as a white solid. MS (M+H)⁺=872.3.

¹H NMR (400 MHz, DMSO-d₆) δ=10.77 (br s, 1H), 9.92 (s, 1H), 9.73 (s, 1H), 9.59 (s, 1H), 8.24 (d, J=3.5 Hz, 1H), 8.08-7.98 (m, 4H), 7.94 (br d, J=7.8 Hz, 1H), 7.80-7.72 (m, 4H), 7.65 (br d, J=7.5 Hz, 1H), 7.56 (dd, J=1.2, 8.0 Hz, 1H), 7.44-7.35 (m, 1H), 7.33-7.24 (m, 1H), 7.03 (d, J=8.7 Hz, 2H), 6.88 (d, J=8.7 Hz, 2H), 3.76-3.58 (m, 4H), 3.30 (s, 3H), 2.65-2.59 (m, 2H), 2.30-2.20 (m, 2H), 2.19-2.17 (m, 3H), 2.15-2.08 (m, 1H), 2.04-1.98 (m, 1H), 1.90-1.83 (m, 2H), 1.81-1.69 (m, 4H), 1.61-1.45 (m, 1H), 1.38-1.26 (m, 4H), 1.20-1.10 (m, 2H).

### Example 50. Synthesis of N-(2-chlorophenyl)-4-[[2-[4-[[1-[[1-[4-(2,6-dioxo-3-piperidyl)phenyl]-4-piperidyl]methyl]-4-piperidyl]carbamoyl]anilino]-5-fluoro-pyrimidin-4-yl]amino]benzamide (Compound 50)

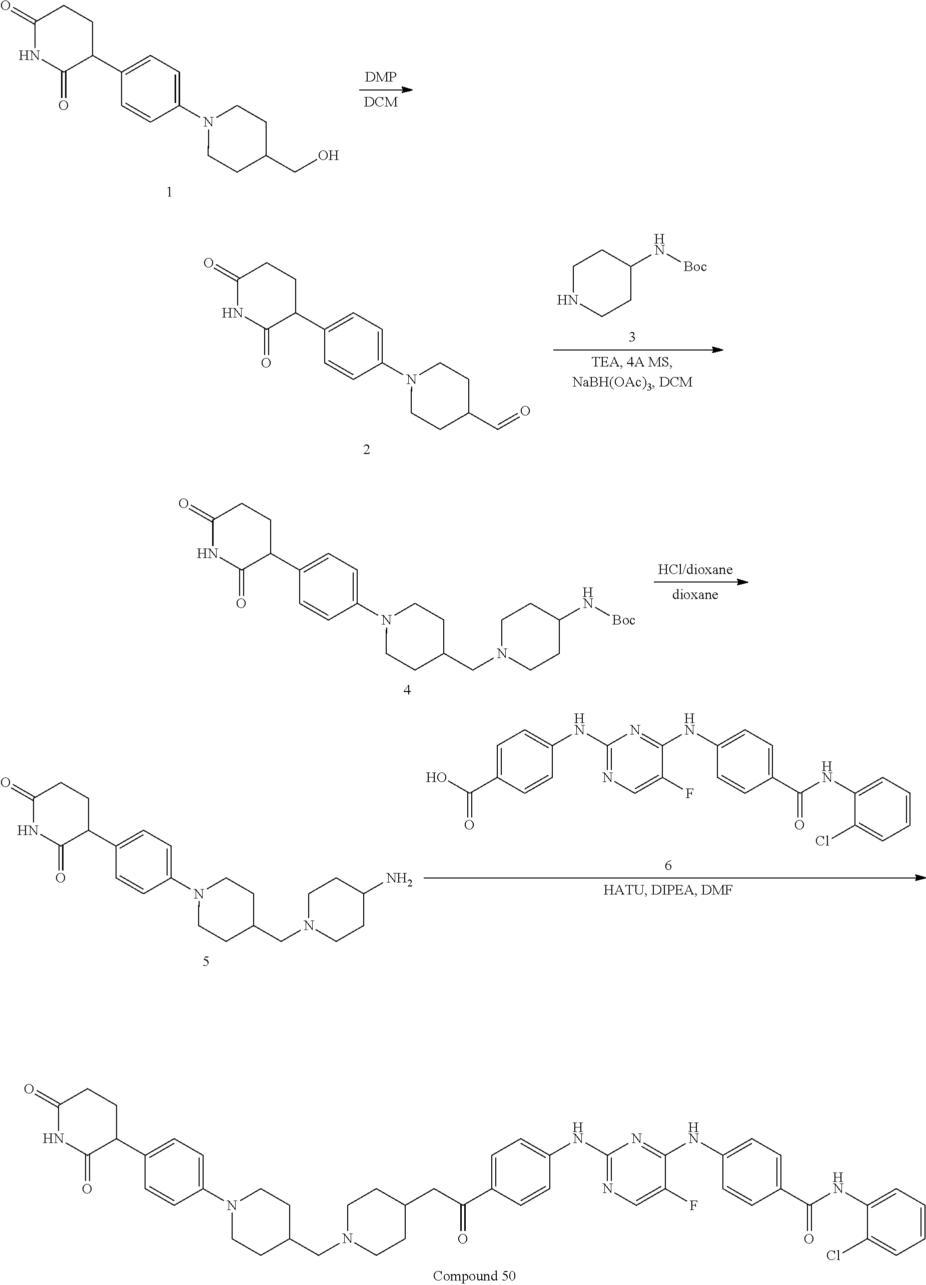

### Step 1. Synthesis of 1-[4-(2,6-dioxo-3-piperidyl)phenyl]piperidine-4-carbaldehyde

To a solution of 3-[4-[4-(hydroxymethyl)-1-piperidyl]phenyl]piperidine-2,6-dione (200 mg, 661.45 μmol) in DCM (1 mL) was added DMP (308.60 mg, 727.59 μmol). The mixture was stirred at 15° C. for 1 hr. TLC (Petroleum ether:EtOAc=1:2) indicated that one new spot (R_{f}=0.3) was formed and starting material remained. The reaction mixture was quenched with aqueous Na₂SO₃ solution (20 mL) and extracted with EtOAc (10 ml×3). The combined organic phase was dried over Na₂SO₄, filtrated and concentrated under reduced pressure. The residue was purified by flash silica gel chromatography (12 g SepaFlash® Silica Flash Column, Eluent of 0~100% EtOAc/Petroleum ether to 0~25% MeOH/EtOAc, gradient @ 60 mL/min) to afford 1-[4-(2,6-dioxo-3-piperidyl)phenyl]piperidine-4-carbaldehyde (195 mg, 649.24 μmol, crude) as a white solid, which was directly used into next step. MS(M+H)⁺=301.4.

### Step 2. Synthesis of tert-butyl N-[1-[[1-[4-(2,6-dioxo-3-piperidyl)phenyl]-4-piperidyl]methyl]-4-piperidyl]carbamate (4)

To a solution of 1-[4-(2,6-dioxo-3-piperidyl)phenyl]piperidine-4-carbaldehyde (195 mg, 649.24 μmol) and tert-butyl N-(4-piperidyl)carbamate (130.03 mg, 649.24 mol) in DCM (2 mL) were added TEA (197.09 mg, 1.95 mmol, 271.10 μL) and 4 Å MS (20 mg). After stirring at 15° C. for 0.5 hr, NaBH(OAc)₃ (412.80 mg, 1.95 mmol) was added and the resulting mixture was stirred at 15° C. for 16 hr. LCMS showed a peak (24%) with the mass of desired product and starting material was consumed. The reaction mixture was diluted with EtOAc (10 mL) and filtered. The filtrate was concentrated under reduced pressure to afford a residue, which was triturated with EtOAc (5 mL) and MeCN (5 mL) to afford tert-butyl N-[1-[[1-[4-(2,6-dioxo-3-piperidyl)phenyl]-4-piperidyl]methyl]-4-piperidyl]carbamate (100 mg, 177.46 μmol, 27.33% yield, 86% purity) as yellow solid. MS(M+H)⁺=485.3.

### Step 3. Synthesis of 3-[4-[4-[(4-amino-1-piperidyl)methyl]-1-piperidyl]phenyl]piperidine-2,6-dione (5)

To a solution of tert-butyl N-[1-[[1-[4-(2,6-dioxo-3-piperidyl)phenyl]-4-piperidyl]methyl]-4-piperidyl]carbamate (100 mg, 206.34 mol) in dioxane (1 mL) was added HCl/dioxane (4 M, 1 mL). The mixture was stirred at 15° C. for 1 hr. LCMS showed a peak (87%) with the mass of desired product. The reaction mixture was concentrated under reduced pressure to afford 3-[4-[4-[(4-amino-1-piperidyl)methyl]-1-piperidyl]phenyl]piperidine-2,6-dione (80 mg, crude, HCl) as yellow solid. MS(M+H)⁺=385.3.

### Step 4. Synthesis of N-(2-chlorophenyl)-4-[[2-[4-[[1-[[1-[4-(2,6-dioxo-3-piperidyl)phenyl]-4-piperidyl]methyl]-4-piperidyl]carbamoyl]anilino]-5-fluoro-pyrimidin-4-yl]amino]benzamide (Compound 50)

To a solution of 3-[4-[4-[(4-amino-1-piperidyl)methyl]-1-piperidyl]phenyl]piperidine-2,6-dione (80 mg, 208.05 μmol), DIPEA (80.67 mg, 624.16 μmol, 108.72 μL) and 4-[[4-[4-[(2-chlorophenyl)carbamoyl]anilino]-5-fluoro-pyrimidin-2-yl]amino]benzoic acid (99.42 mg, 208.05 μmol) in DMF (2 mL) was added HATU (118.66 mg, 312.08 μmol). The mixture was stirred at 15° C. for 16 hr. LCMS showed a peak (34%) with the mass of desired product. The reaction mixture was diluted with water (15 mL) and filtered. The residue was purified by prep-HPLC(column: Phenomenex luna C18 150*25 mm*10 um; mobile phase: [water(TFA)-ACN]; gradient: 23%-53% B over 10 min) and lyophilized to afford the crude product, which was further purified by prep-HPLC (column: Waters Xbridge 150*25 mm*5 um; mobile phase: [water(NH₄HCO₃)-ACN]; gradient: 30%-60% B over 10 min) and lyophilized to afford N-(2-chlorophenyl)-4-[[2-[4-[[1-[[1-[4-(2,6-dioxo-3-piperidyl)phenyl]-4-piperidyl]methyl]-4-piperidyl]carbamoyl]anilino]-5-fluoro-pyrimidin-4-yl]amino]benzamide (8.5 mg, 9.87 μmol, 4.74% yield, 98% purity) was obtained as white solid. MS(M+H)⁺=844.3.

¹H NMR (400 MHz, DMSO-d₆) δ=10.77 (s, 1H), 9.93 (s, 1H), 9.75 (s, 1H), 9.61 (s, 1H), 8.26 (d, J=3.3 Hz, 1H), 8.09-7.97 (m, 5H), 7.80-7.75 (m, 4H), 7.67 (br d, J=8.2 Hz, 1H), 7.57 (d, J=7.8 Hz, 1H), 7.41 (br t, J=7.3 Hz, 1H), 7.34-7.27 (m, 1H), 7.04 (br d, J=8.6 Hz, 2H), 6.89 (br d, J=8.9 Hz, 2H), 3.83-3.61 (m, 5H), 2.90-2.80 (m, 2H), 2.65-2.55 (m, 3H), 2.20-2.13 (m, 2H), 2.06-1.90 (m, 3H), 1.84-1.72 (m, 4H), 1.68-1.54 (m, 3H), 1.28-1.12 (m, 3H).

### Example 51. Synthesis of N-(2-chlorophenyl)-4-((2-((4-((1-(2-(4-(4-(2,6-dioxopiperidin-3-yl)phenyl)piperazin-1-yl)-2-oxoethyl)piperidin-4-yl)carbamoyl)phenyl)amino)-5-fluoropyrimidin-4-yl)amino)benzamide (Compound 51)

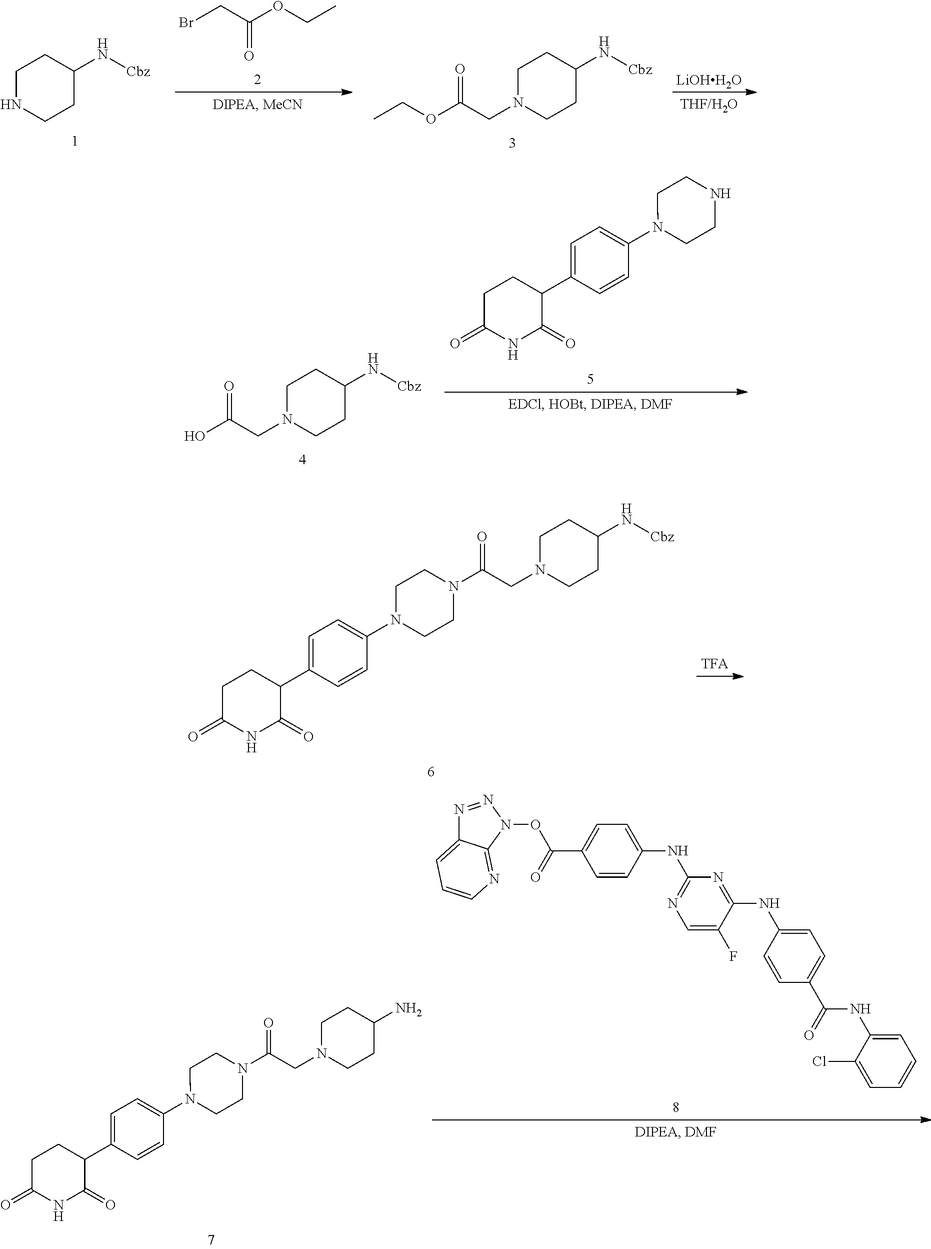

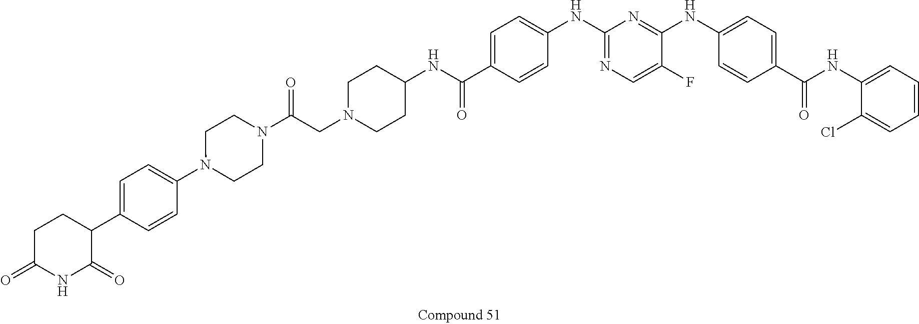 [SMILES: O=C1CCC(c2ccc(N3CCN(C(=O)CN4CCC(NC(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of ethyl 2-(4-(((benzyloxy)carbonyl)amino)piperidin-1-yl)acetate (3)

To a solution of benzyl piperidin-4-ylcarbamate (1 g, 4.27 mmol) and ethyl 2-bromoacetate (926.62 mg, 5.55 mmol, 614.06 μL) in MeCN (10 mL) was added DIPEA (1.65 g, 12.80 mmol, 2.23 mL). The mixture was stirred at 80° C. for 16 hr. LCMS showed benzyl piperidin-4-ylcarbamate was consumed completely. The reaction mixture was diluted with H₂O (15 mL) at 20° C., and then extracted with EtOAc (50 mL×2). The combined organic layers were dried over Na₂SO₄, filtered and concentrated under reduced pressure to give a residue, which was purified by flash silica gel chromatography (ISCO®; 20 g SepaFlash® Silica Flash Column, Eluent of 0-40% EtOAc/Petroleum ether gradient @ 60 mL/min) to afford ethyl 2-(4-(((benzyloxy)carbonyl)amino)piperidin-1-yl)acetate (400 mg, 1.25 mmol, 29.25% yield) as a yellow solid. MS(M+H)⁺=321.2.

### Step 2. Synthesis of 2-(4-(((benzyloxy)carbonyl)amino)piperidin-1-yl)acetic acid (4)

To a solution of ethyl 2-(4-(((benzyloxy)carbonyl)amino)piperidin-1-yl)acetate (0.4 g, 1.25 mmol) in THF (4 mL) was added LiOH·H₂O (157.18 mg, 3.75 mmol) in H₂O (1 mL). The mixture was stirred at 25° C. for 16 hr. LCMS showed ethyl 2-(4-(((benzyloxy)carbonyl)amino)piperidin-1-yl)acetate was consumed completely. The reaction mixture was concentrated under reduced pressure to afford 2-(4-(((benzyloxy)carbonyl)amino)piperidin-1-yl)acetic acid (370 mg, 1.24 mmol, 99.03% yield, Li salt) as a white solid. MS(M+H)⁺=293.1.

### Step 3. Synthesis of benzyl (1-(2-(4-(4-(2,6-dioxopiperidin-3-yl)phenyl)piperazin-1-yl)-2-oxoethyl)piperidin-4-yl) carbamate (6)

To a solution of 3-(4-(piperazin-1-yl)phenyl)piperidine-2,6-dione (340.15 mg, 982.39 μmol, 2HCl) and 2-(4-(((benzyloxy)carbonyl)amino)piperidin-1-yl)acetic acid (294 mg, 982.39 μmol, Li) in DMF (3.5 mL) was added EDCI (282.49 mg, 1.47 mmol), DIPEA (634.82 mg, 4.91 mmol, 855.55 μL) and HOBt (132.74 mg, 982.39 mol). The mixture was stirred at 40° C. for 16 hr. LCMS showed 43% of 2-(4-(((benzyloxy)carbonyl)amino)piperidin-1-yl)acetic acid remained and 31% of desired mass was detected. And then another portion of EDCI (94.16 mg, 491.19 mol) and HOBt (66.37 mg, 491.19 μmol) were added and the mixture and stirred at 40° C. for 4 hr. LCMS showed the reaction was completed. The reaction mixture was diluted with H₂O (20 mL) at 25° C., and then extracted with EtOAc (10 mL×2). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0~20% MeOH/EtOAc @ 50 mL/min) to afford benzyl (1-(2-(4-(4-(2,6-dioxopiperidin-3-yl)phenyl)piperazin-1-yl)-2-oxoethyl)piperidin-4-yl) carbamate (130 mg, 201.77 μmol, 20.54% yield, 85% purity) was obtained as a white solid. MS(M+H)⁺=548.4.

### Step 4. Synthesis of 3-(4-(4-(2-(4-aminopiperidin-1-yl)acetyl)piperazin-1-yl)phenyl)piperidine-2,6-dione (7)

A solution of benzyl (1-(2-(4-(4-(2,6-dioxopiperidin-3-yl)phenyl)piperazin-1-yl)-2-oxoethyl)piperidin-4-yl) carbamate (50 mg, 77.61 μmol) in TFA (767.50 mg, 6.73 mmol, 0.5 mL) was stirred at 40° C. for 16 hr. LCMS showed benzyl (1-(2-(4-(4-(2,6-dioxopiperidin-3-yl)phenyl)piperazin-1-yl)-2-oxoethyl)piperidin-4-yl) carbamate was consumed completely and desired mass was detected. The reaction mixture was concentrated under reduced pressure to afford 3-(4-(4-(2-(4-aminopiperidin-1-yl)acetyl)piperazin-1-yl)phenyl)piperidine-2,6-dione (40 mg, 75.82 μmol, 97.71% yield, TFA) as a yellow oil. MS(M+H)⁺=414.2.

### Step 5. Synthesis of N-(2-chlorophenyl)-4-((2-((4-((1-(2-(4-(4-(2,6-dioxopiperidin-3-yl)phenyl)piperazin-1-yl)-2-oxoethyl)piperidin-4-yl)carbamoyl)phenyl)amino)-5-fluoropyrimidin-4-yl)amino)benzamide (Compound 51)

To a solution of 3-(4-(4-(2-(4-aminopiperidin-1-yl)acetyl)piperazin-1-yl)phenyl)piperidine-2,6-dione (40 mg, 75.82 μmol, TFA) and 3H-[1,2,3]triazolo[4,5-b]pyridin-3-yl 4-((4-((4-((2-chlorophenyl)carbamoyl)phenyl)amino)-5-fluoropyrimidin-2-yl)amino)benzoate (45.19 mg, 75.82 μmol) in DMF (0.4 mL) was added DIPEA (49.00 mg, 379.12 mol, 66.04 μL). The mixture was stirred at 25° C. for 1 hr. LCMS showed 3-(4-(4-(2-(4-aminopiperidin-1-yl)acetyl)piperazin-1-yl)phenyl)piperidine-2,6-dione was consumed completely and desired mass was detected. The reaction mixture was diluted with H₂O (20 mL) at 25° C., and then extracted with EtOAc (10 mL×2). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0~30% MeOH/EtOAc @ 50 mL/min). The impure product was purified by prep-HPLC (column: Waters Xbridge 150*25 mm 10 um; mobile phase: [water (NH₄ HCO₃)-ACN]; gradient: 32%-62% B over 10 min) and lyophilized to afford N-(2-chlorophenyl)-4-((2-((4-((1-(2-(4-(4-(2,6-dioxopiperidin-3-yl)phenyl)piperazin-1-yl)-2-oxoethyl)piperidin-4-yl)carbamoyl)phenyl)amino)-5-fluoropyrimidin-4-yl)amino)benzamide (11.2 mg, 12.57 μmol, 16.57% yield, 98% purity) as a white solid. MS(M+H)⁺=873.5.

¹H NMR (400 MHz, DMSO-d₆) δ=10.78 (s, 1H), 9.92 (s, 1H), 9.74 (s, 1H), 9.60 (s, 1H), 8.24 (d, J=3.5 Hz, 1H), 8.08-7.96 (m, 5H), 7.78-7.72 (m, 4H), 7.65 (d, J=7.6 Hz, 1H), 7.56 (d, J=7.8 Hz, 1H), 7.39 (t, J=7.6 Hz, 1H), 7.33-7.25 (m, 1H), 7.07 (br d, J=8.6 Hz, 2H), 6.93 (br d, J=8.4 Hz, 2H), 3.80-3.66 (m, 4H), 3.65-3.55 (m, 2H), 3.25-3.10 (m, 4H), 3.10-3.00 (m, 2H), 2.84 (br d, J=10.8 Hz, 2H), 2.67-2.62 (m, 1H), 2.45-2.40 (m, 1H), 2.19-2.04 (m, 3H), 2.02-1.94 (m, 1H), 1.82-1.69 (m, 2H), 1.60-1.55 (m, 2H).

### Example 52. Synthesis of N-(2-chlorophenyl)-4-((2-((4-((4-(3-(4-(4-(2,6-dioxopiperidin-3-yl)phenyl)piperazin-1-yl)propyl)piperidin-1-yl)carbamoyl)phenyl)amino)-5-fluoropyrimidin-4-yl)amino)benzamide (Compound 52)

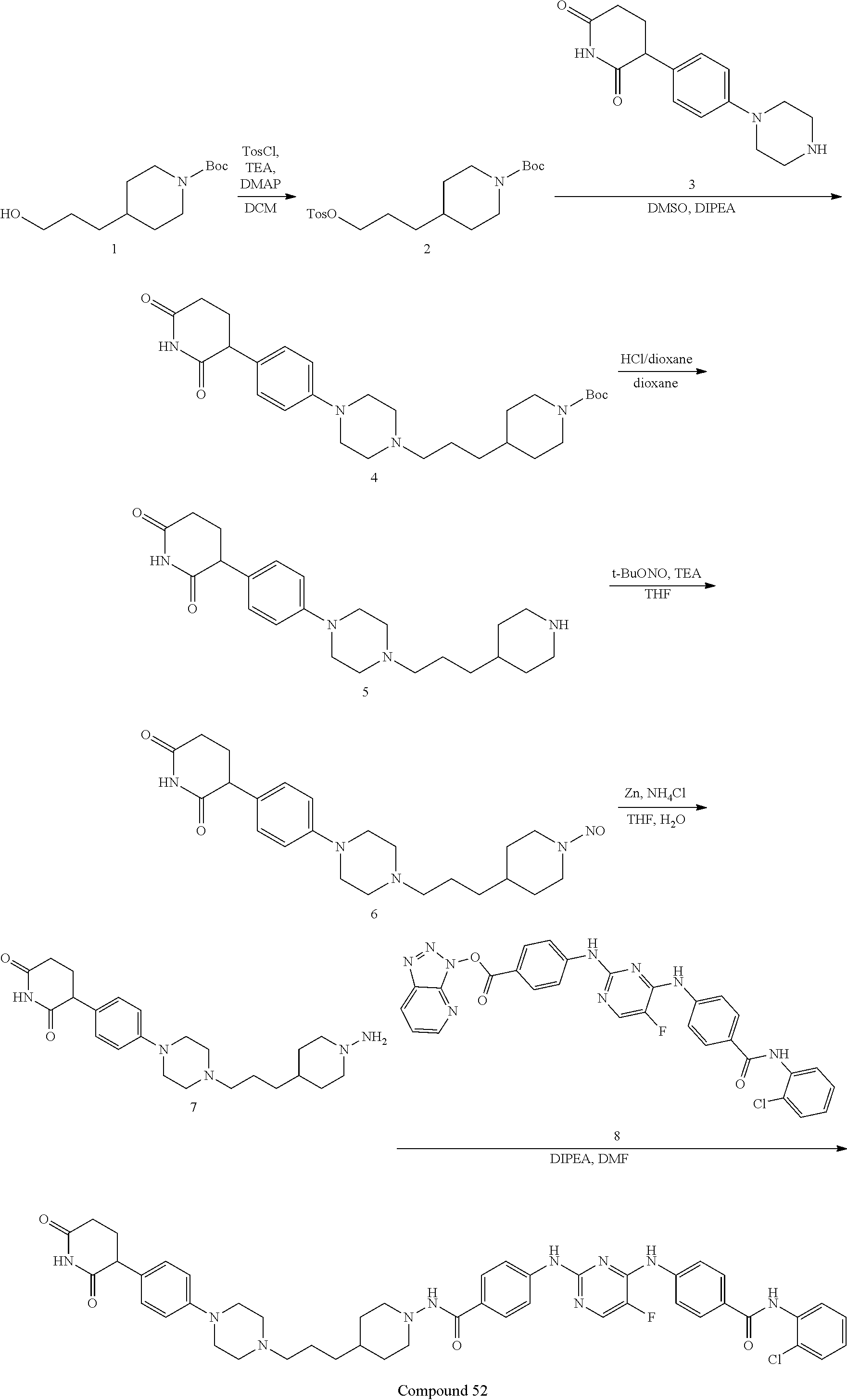

### Step 1. Synthesis of tert-butyl 4-(3-(tosyloxy)propyl)piperidine-1-carboxylate (2)

To a solution of tert-butyl 4-(3-hydroxypropyl)piperidine-1-carboxylate (1 g, 4.11 mmol) in DCM (10 mL) was added TEA (1.25 g, 12.33 mmol, 1.72 mL), DMAP (50.20 mg, 410.95 mol) and TosCl (1.18 g, 6.16 mmol). The mixture was stirred at 25° C. for 16 hr. LCMS showed tert-butyl 4-(3-hydroxypropyl)piperidine-1-carboxylate was consumed completely and desired mass was detected. The reaction mixture was diluted with H₂O (20 mL) and extracted with EtOAc (10 mL×2). The combined organic layers were dried over Na₂SO₄, filtered and concentrated under reduced pressure to give a residue, which was purified by flash silica gel chromatography (ISCO®; 20 g SepaFlash® Silica Flash Column, Eluent of 0~20% EtOAc/Petroleum ether, gradient @ 80 mL/min) to afford tert-butyl 4-(3-(tosyloxy)propyl)piperidine-1-carboxylate (1.44 g, 3.55 mmol, 86.38% yield, 98% purity) as a yellow oil. MS(M−Boc+H)⁺=298.2.

### Step 2. Synthesis of tert-butyl 4-(3-(4-(4-(2,6-dioxopiperidin-3-yl)phenyl)piperazin-1-yl)propyl)piperidine-1-carboxylate (4)

To a solution of 3-(4-(piperazin-1-yl)phenyl)piperidine-2,6-dione (950 mg, 3.48 mmol, 2HCl) and tert-butyl 4-(3-(tosyloxy)propyl)piperidine-1-carboxylate (1.44 g, 3.55 mmol) in DMSO (10 mL) was added DIPEA (1.35 g, 10.43 mmol, 1.82 mL). The mixture was stirred at 100° C. for 16 hr. LCMS showed 3-(4-(piperazin-1-yl)phenyl)piperidine-2,6-dione was consumed completely and desired mass was detected. The reaction mixture was diluted with H₂O (20 mL) at 25° C., and then extracted with EtOAc (10 mL×2). The combined organic layers were washed with brine (15 mL), dried over Na₂SO₄, filtered and concentrated under reduced pressure to give a residue, which was purified by flash silica gel chromatography (ISCO®; 20 g SepaFlash® Silica Flash Column, Eluent of 0~100% EtOAc/Petroleum ether, gradient @ 80 mL/min) to afford tert-butyl 4-(3-(4-(4-(2,6-dioxopiperidin-3-yl)phenyl)piperazin-1-yl)propyl)piperidine-1-carboxylate (660 mg, 1.09 mmol, 31.23% yield, 82% purity) as a yellow solid. MS(M+H)⁺=499.3.

### Step 3. Synthesis of 3-(4-(4-(3-(piperidin-4-yl)propyl)piperazin-1-yl)phenyl)piperidine-2,6-dione (5)

To a solution of tert-butyl 4-(3-(4-(4-(2,6-dioxopiperidin-3-yl)phenyl)piperazin-1-yl)propyl)piperidine-1-carboxy late (660 mg, 1.09 mmol) in dioxane (6.5 mL) was added HCl/dioxane (4 M, 13 mL). The mixture was stirred at 25° C. for 1 hr. LCMS showed the starting material was consumed completely and desired mass was detected. The reaction mixture was concentrated under reduced pressure to afford 3-(4-(4-(3-(piperidin-4-yl)propyl)piperazin-1-yl)phenyl)piperidine-2,6-dione (470 mg, 1.08 mmol, 99.55% yield, HCl) as a yellow solid. MS(M+H)⁺=399.4.

### Step 4. Synthesis of 3-(4-(4-(3-(1-nitrosopiperidin-4-yl)propyl)piperazin-1-yl)phenyl)piperidine-2,6-dione (6)

To a solution of 3-(4-(4-(3-(piperidin-4-yl)propyl)piperazin-1-yl)phenyl)piperidine-2,6-dione (0.45 g, 1.03 mmol, HCl) in THF (9 mL) was added TEA (732.75 mg, 7.24 mmol, 1.01 mL) at 0° C., followed by the addition of t-BuONO (160.01 mg, 1.55 mmol, 184.56 μL) at 25° C. The resulting mixture was stirred at 60° C. for 48 hr. LCMS showed 28% of 3-(4-(4-(3-(piperidin-4-yl)propyl)piperazin-1-yl)phenyl)piperidine-2,6-dione remained and a peak of 54% was desired mass. Another portion of t-BuONO (213.35 mg, 2.07 mmol, 246.08 μL) was added and the mixture was stirred at 60° C. for 16 hr. LCMS showed 3-(4-(4-(3-(piperidin-4-yl)propyl)piperazin-1-yl)phenyl)piperidine-2,6-dione was consumed completely. The reaction mixture was diluted with H₂O (30 mL) at 25° C. and extracted with EtOAc (10 mL×2). The combined organic layers were dried over Na₂SO₄, filtered and concentrated under reduced pressure to give a residue. The residue was purified by flash silica gel chromatography (ISCO®; 20 g SepaFlash® Silica Flash Column, Eluent of 0~20% MeOH/EtOAc @ 80 mL/min) to afford 3-(4-(4-(3-(1-nitrosopiperidin-4-yl)propyl)piperazin-1-yl)phenyl)piperidine-2,6-dione (450 mg, 926.23 μmol, 89.54% yield, 88% purity) as a brown solid. MS(M+H)⁺=428.2.

### Step 5. Synthesis of 3-(4-(4-(3-(1-aminopiperidin-4-yl)propyl)piperazin-1-yl)phenyl)piperidine-2,6-dione (7)

To a solution of 3-(4-(4-(3-(1-nitrosopiperidin-4-yl)propyl)piperazin-1-yl)phenyl)piperidine-2,6-dione (200 mg, 467.79 mol) and NH₄Cl (75.07 mg, 1.40 mmol) in THF (4 mL) and H₂O (2 mL) was added Zn (91.77 mg, 1.40 mmol) at 0° C. The mixture was stirred at 20° C. for 16 hr. LC-MS showed 23% of 3-(4-(4-(3-(1-nitrosopiperidin-4-yl)propyl)piperazin-1-yl)phenyl)piperidine-2,6-dione remained and a peak of 62% was desired mass. The reaction mixture filtered and the filter cake was washed with THF (80 mL). The filtrate was concentrated under reduced pressure to afford 3-(4-(4-(3-(1-aminopiperidin-4-yl)propyl)piperazin-1-yl)phenyl)piperidine-2,6-dione (180 mg, 435.25 μmol, 93.04% yield) as a white solid. MS(M+H)⁺=414.3.

### Step 6. Synthesis of N-(2-chlorophenyl)-4-((2-((4-((4-(3-(4-(4-(2,6-dioxopiperidin-3-yl)phenyl)piperazin-1-yl)propyl)piperidin-1-yl)carbamoyl)phenyl)amino)-5-fluoropyrimidin-4-yl) amino)benzamide (Compound 52)

To a solution of 3-(4-(4-(3-(1-aminopiperidin-4-yl)propyl)piperazin-1-yl)phenyl)piperidine-2,6-dione (100 mg, 241.81 mol) and 3H-[1,2,3]triazolo[4,5-b]pyridin-3-yl 4-((4-((4-((2-chlorophenyl)carbamoyl)phenyl)amino)-5-fluoropyrimidin-2-yl)amino)benzoate (108.47 mg, 169.26 mol) in DMF (1 mL) was added DIPEA (156.26 mg, 1.21 mmol, 210.59 μL). The mixture was stirred at 25° C. for 16 hr. LCMS showed 3-(4-(4-(3-(1-aminopiperidin-4-yl)propyl)piperazin-1-yl)phenyl)piperidine-2,6-dione was consumed completely. The reaction mixture was purified by prep-HPLC (column: Waters xbridge 150*25 mm 10 um; mobile phase: [water (NH₄HCO₃)-ACN]; gradient: 26%-56% B over 10 min) and lyophilized to afford N-(2-chlorophenyl)-4-((2-((4-((4-(3-(4-(4-(2,6-dioxopiperidin-3-yl)phenyl)piperazin-1-yl)propyl)piperidin-1-yl)carbamoyl)phenyl)amino)-5-fluoropyrimidin-4-yl)amino)benzamide (5.2 mg, 5.72 μmol, 2.36% yield, 96% purity) as a white solid. MS(M+H)⁺=873.5.

¹H NMR (400 MHz, DMSO-d₆) δ=10.77 (s, 1H), 9.93 (s, 1H), 9.74 (s, 1H), 9.65-9.56 (m, 1H), 9.13 (s, 1H), 8.24 (d, J=3.5 Hz, 1H), 8.10-7.94 (m, 4H), 7.81-7.73 (m, 2H), 7.72-7.61 (m, 3H), 7.60-7.53 (m, 1H), 7.43-7.36 (m, 1H), 7.33-7.25 (m, 1H), 7.05 (br d, J=8.5 Hz, 2H), 6.90 (br d, J=8.3 Hz, 2H), 3.79-3.68 (m, 1H), 3.22-3.02 (m, 4H), 3.01-2.93 (m, 2H), 2.75-2.63 (m, 3H), 2.63-2.55 (m, 2H), 2.48-2.41 (m, 2H), 2.40-2.28 (m, 2H), 2.27-2.09 (m, 2H), 2.05-1.93 (m, 1H), 1.72-1.62 (m, 2H), 1.58-1.42 (m, 2H), 1.32-1.18 (m, 5H).

### Example 53. Synthesis of N-(4-((4-((4-((2-chlorophenyl)carbamoyl)phenyl)amino)-5-fluoropyrimidin-2-yl)amino)phenyl)-4-(2-(4-(4-(2,6-dioxopiperidin-3-yl)phenyl)piperazin-1-yl)ethyl)piperidine-1-carboxamide (Compound 53)

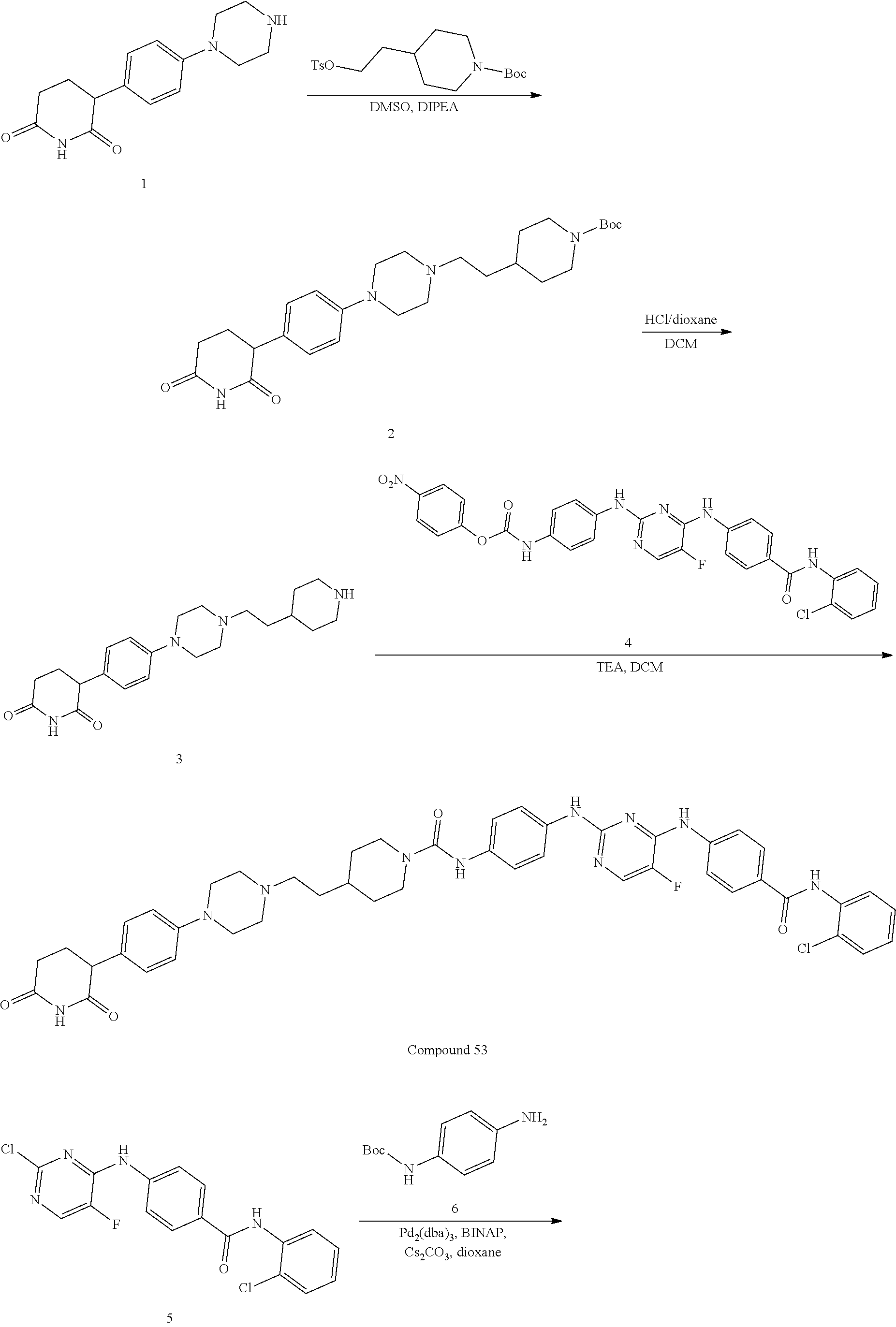

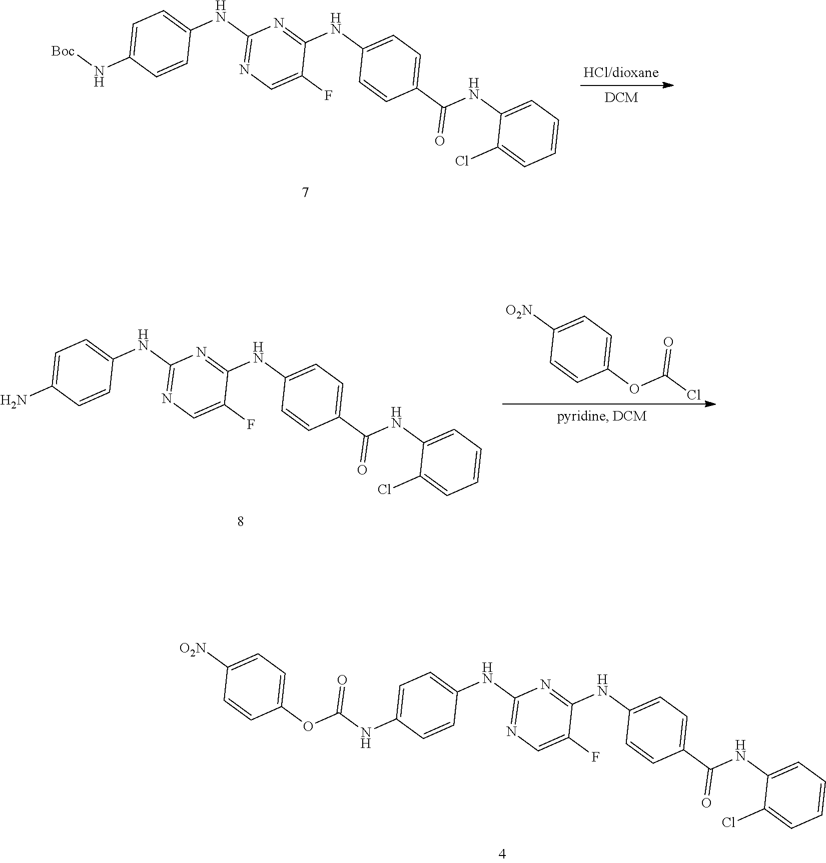 [SMILES: CC(C)(C)OC(=O)Nc1ccc(Nc2ncc(F)c(Nc3ccc(C(=O)Nc4ccccc4Cl)cc3)n2)cc1 ; Nc1ccc(Nc2ncc(F)c(Nc3ccc(C(=O)Nc4ccccc4Cl)cc3)n2)cc1 ; O=C(Cl)Oc1ccc([N+](=O)[O-])cc1 ; O=C(Nc1ccc(Nc2ncc(F)c(Nc3ccc(C(=O)Nc4ccccc4Cl)cc3)n2)cc1)Oc1ccc([N+](=O)[O-])cc1]

### Step 1. Synthesis of tert-butyl 4-(2-(4-(4-(2,6-dioxopiperidin-3-yl)phenyl) piperazin-1-yl)ethyl) piperidine-1-carboxylate (2)

To a solution of tert-butyl 4-(2-(tosyloxy)ethyl) piperidine-1-carboxylate (561.23 mg, 1.46 mmol) and 3-(4-(piperazin-1-yl)phenyl) piperidine-2,6-dione (400 mg, 1.46 mmol) in DMSO (5 mL) was added DIPEA (189.14 mg, 1.46 mmol, 254.90 μL). The mixture was stirred at 100° C. for 43 hours. LCMS showed tert-butyl 4-(2-(tosyloxy)ethyl) piperidine-1-carboxylate was consumed completely and 34% of desired compound was detected. The residue was diluted with H₂O (30 mL) and extracted with solvent EtOAc (20 mL×3). The combined organic layers were washed with brine (60 mL), dried over Na₂SO₄, filtered and concentrated under reduced pressure to give a residue. The residue was purified by flash silica gel chromatography (20 g SepaFlash® Silica Flash Column, Eluent of 0~100% EtOAc/Petroleum ether gradient @ 75 mL/min) to afford tert-butyl 4-(2-(4-(4-(2,6-dioxopiperidin-3-yl)phenyl) piperazin-1-yl)ethyl) piperidine-1-carboxylate (153 mg, 277.82 μmol, 18.98% yield, 88% purity) as a yellow solid. MS (M+H)⁺=485.3.

### Step 2. Synthesis of 3-(4-(4-(2-(piperidin-4-yl)ethyl) piperazin-1-yl)phenyl) piperidine-2,6-dione (3)

To a solution of tert-butyl 4-(2-(4-(4-(2,6-dioxopiperidin-3-yl)phenyl) piperazin-1-yl)ethyl) piperidine-1-carboxylate (153 mg, 315.71 μmol) in DCM (10 mL) was added HCl/dioxane (4 M, 78.93 μL). The mixture was stirred at 25° C. for 2 hr. LCMS showed the starting material was consumed completely and 80% of desired mass was detected. The reaction mixture was concentrated in vacuum to afford 3-(4-(4-(2-(piperidin-4-yl)ethyl)piperazin-1-yl)phenyl)piperidine-2,6-dione (188 mg, HCl salt) as a brown solid, which was used directly. MS(M+H)⁺=385.3.

### Step 3. Synthesis of tert-butyl (4-((4-((4-((2-chlorophenyl)carbamoyl)phenyl)amino)-5-fluoropyrimidin-2-yl)amino)phenyl)carbamate (7)

To a solution of tert-butyl (4-aminophenyl)carbamate (1 g, 4.80 mmol) and 4-((2-chloro-5-fluoropyrimidin-4-yl)amino)-N-(2-chlorophenyl)benzamide (2.17 g, 5.76 mmol) in dioxane (30 mL) were added Pd₂(dba)₃ (219.85 mg, 240.09 μmol), Cs₂CO₃ (4.69 g, 14.41 mmol) and BINAP (298.99 mg, 480.18 μmol). The mixture was stirred at 95° C. for 14 hr under N₂. LCMS showed 10% of 4-((2-chloro-5-fluoropyrimidin-4-yl)amino)-N-(2-chlorophenyl)benzamide remained and 15% of desired compound was detected. Pd₂(dba)₃ (219.85 mg, 240.09 μmol) and BINAP (298.99 mg, 480.18 μmol) were added and the resulting mixture was stirred at 95° C. for further 14 hr under N₂. LCMS showed 22% of desired compound was detected. The residue was diluted with H₂O (50 mL) and extracted with EtOAc (20 mL×3). The combined organic layers were washed with brine (100 mL), dried over Na₂SO₄, filtered and concentrated in vacuum. The residue was purified by flash silica gel chromatography (80 g SepaFlash® Silica Flash Column, Eluent of 0~50% EtOAc/Petroleum ether gradient @ 80 mL/min) to afford tert-butyl (4-((4-((4-((2-chlorophenyl)carbamoyl)phenyl)amino)-5-fluoropyrimidin-2-yl)amino)phenyl)carbamate (1.3 g, 1.99 mmol, 41.42% yield, 84% purity) as a yellow solid. MS(M+H)⁺=549.3.

### Step 4. Synthesis of 4-((2-((4-aminophenyl)amino)-5-fluoropyrimidin-4-yl)amino)-N-(2-chlorophenyl)benzamide (8)

To a solution of tert-butyl (4-((4-((4-((2-chlorophenyl)carbamoyl)phenyl)amino)-5-fluoropyrimidin-2-yl)amino)phenyl)carbamate (1.3 g, 2.37 mmol) in DCM (20 mL) was added HCl/dioxane (4 M, 591.99 μL), the mixture was stirred at 25° C. for 5.5 hours. LCMS showed the starting material was consumed completely and 90% of desired compound was detected. The mixture was concentrated in vacuum to afford 4-((2-((4-aminophenyl)amino)-5-fluoropyrimidin-4-yl)amino)-N-(2-chlorophenyl)benzamide (1.25 g, HCl salt) as a brown solid, which was used directly. MS(M+H)⁺=449.2.

### Step 5. Synthesis of 4-nitrophenyl (4-((4-((4-((2-chlorophenyl)carbamoyl)phenyl)amino)-5-fluoropyrimidin-2-yl)amino)phenyl)carbamate (4)

To a solution of 4-nitrophenyl carbonochloridate (573.12 mg, 2.84 mmol) and 4-((2-((4-aminophenyl)amino)-5-fluoropyrimidin-4-yl)amino)-N-(2-chlorophenyl)benzamide (1.15 g, 2.37 mmol, HCl salt) in DCM (10 mL) was added Py (562.27 mg, 7.11 mmol, 573.75 μL). The mixture was stirred at 25° C. for 4 hours. LCMS showed 4-((2-((4-aminophenyl)amino)-5-fluoropyrimidin-4-yl)amino)-N-(2-chlorophenyl)benzamide was consumed completely. The reaction mixture was poured into water and filtered, the filter cake was washed with methylene chloride, the filter cake was collected and dried in vacuum to afford 4-nitrophenyl (4-((4-((4-((2-chlorophenyl)carbamoyl)phenyl)amino)-5-fluoropyrimidin-2-yl) amino)phenyl)carbamate (1.25 g, 1.85 mmol, 78.19% yield, 91% purity) as a yellow solid, which was used directly. MS(M+H)⁺=614.2.

### Step 6. Synthesis of N-(4-((4-((4-((2-chlorophenyl)carbamoyl)phenyl)amino)-5-fluoropyrimidin-2-yl)amino)phenyl)-4-(2-(4-(4-(2,6-dioxopiperidin-3-yl)phenyl)piperazin-1-yl)ethyl)piperidine-1-carboxamide (Compound 53)

To a solution of 3-(4-(4-(2-(piperidin-4-yl)ethyl)piperazin-1-yl)phenyl)piperidine-2,6-dione (60 mg, 142.53 mol, HCl salt) and 4-nitrophenyl (4-((4-((4-((2-chlorophenyl)carbamoyl)phenyl) amino)-5-fluoropyrimidin-2-yl)amino)phenyl)carbamate (87.51 mg, 142.53 μmol) in DCM (3 mL) was added TEA (43.27 mg, 427.58 mol, 59.51 μL). The mixture was stirred at 25° C. for 1 hr. LCMS showed 66% of desired compound was detected. The mixture was filtered and the filter cake was purified by prep-HPLC (column: Waters Xbridge 150*25 mm*5 um; mobile phase: [water(NH₄HCO₃)-ACN]; gradient: 32%-62% B over min) and lyophilized. The product was triturated with a mixture (MTBE/EtOAc/THF=5/5/1, 11 mL) for 10 minutes, the suspension was filtered and the filter cake was washed with THF (0.5 mL), the filter cake was collected and dried. The solid was diluted with ACN (5 mL), THF (5 mL) and H₂O (15 mL), the mixture was freeze-dried. The residue was triturated with a mixture (EtOAc/THF=5/1, 2 mL) for 10 minutes, the suspension was filtered and the filter cake was washed with a mixture (ACN/THF=5/1, 2 mL). The filter cake was collected and dried. The residue was diluted with a mixture (ACN/THF/H₂O=1/1/5, 21 mL) and freeze-dried to afford N-(4-((4-((4-((2-chlorophenyl) carbamoyl)phenyl)amino)-5-fluoropyrimidin-2-yl)amino)phenyl)-4-(2-(4-(4-(2,6-dioxopiperidin-3-yl)phenyl)piperazin-1-yl)ethyl)piperidine-1-carboxamide (16.2 mg, 17.34 mol, 30.29% yield, 92% purity) as a gray solid. MS(M+H)⁺=859.3.

¹H NMR (400 MHz, DMSO-d₆) δ=10.88-10.66 (m, 1H), 9.86 (br d, J=1.2 Hz, 1H), 9.70-9.48 (m, 1H), 9.22-9.00 (m, 1H), 8.41-8.24 (m, 1H), 8.15 (br dd, J=1.3, 8.1 Hz, 1H), 8.07-7.81 (m, 4H), 7.71-7.60 (m, 1H), 7.59-7.53 (m, 1H), 7.53-7.43 (m, 2H), 7.43-7.23 (m, 4H), 7.14-6.98 (m, 2H), 6.89 (br dd, J=1.2, 3.9 Hz, 2H), 4.22-3.92 (m, 2H), 3.81-3.69 (m, 1H), 3.68-3.49 (m, 2H), 3.30-3.10 (m, 4H), 2.80-2.62 (m, 4H), 2.38-2.28 (m, 2H), 2.16-2.09 (m, 1H), 2.05-1.96 (m, 1H), 1.86-1.70 (m, 2H), 1.70-1.57 (m, 2H), 1.54-1.34 (m, 3H), 1.16-0.98 (m, 2H).

### Example 54. Synthesis of N-(2-chlorophenyl)-4-[[2-[4-[[4-[2-[4-[4-(2,4-dioxohexahydropyrimidin-1-yl)phenyl]piperazin-1-yl]ethyl]-1-piperidyl]carbamoyl]anilino]-5-fluoro-pyrimidin-4-yl]amino]benzamide (Compound 54)

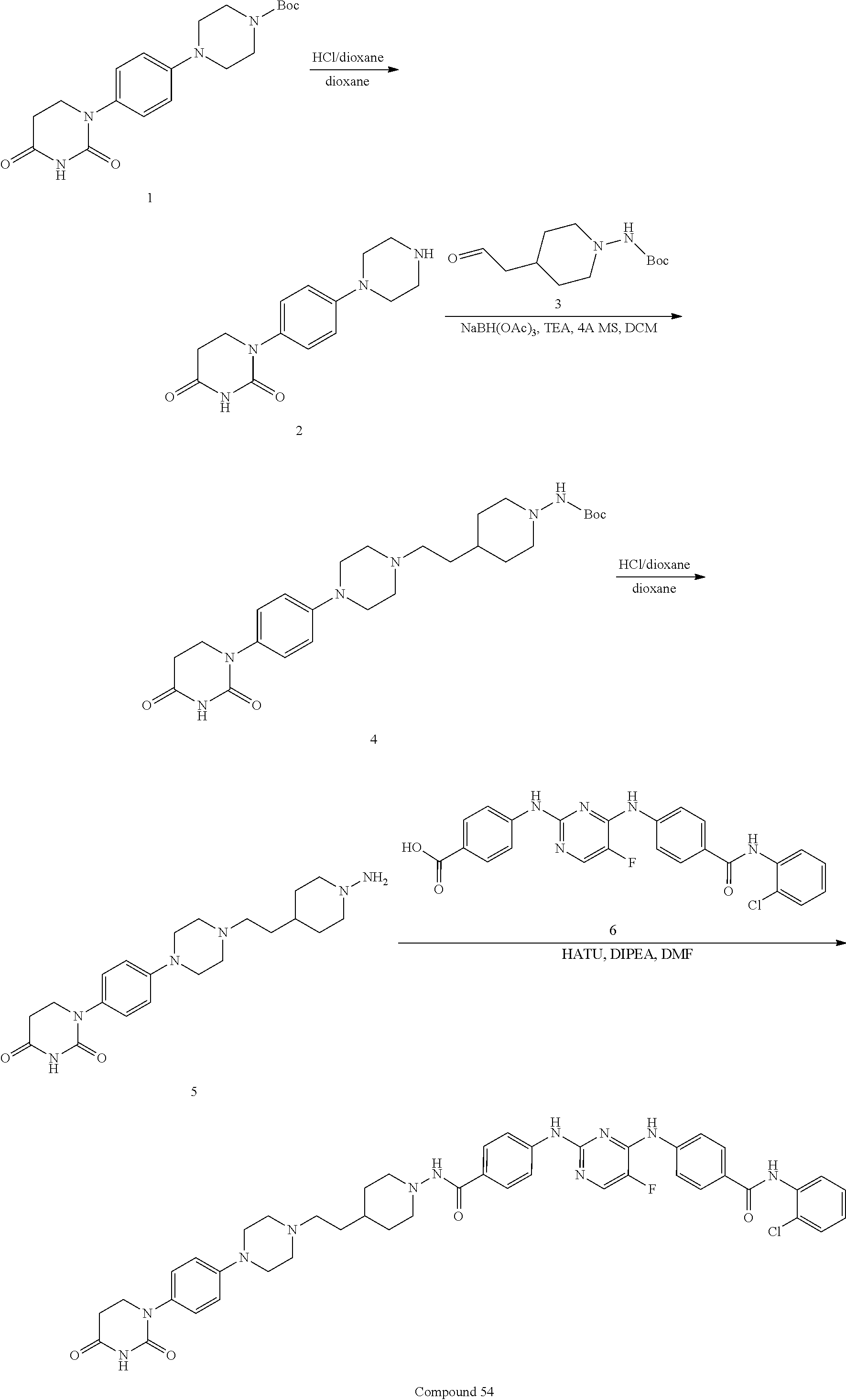

### Step 1. Synthesis of 1-(4-piperazin-1-ylphenyl)hexahydroprimidine-2,4-dion (2)

To a solution of tert-butyl 4-[4-(2,4-dioxohexahydropyrimidin-1-yl)phenyl]piperazine-1-carboxylate (500 mg, 1.34 mmol) in dioxane (5 mL) was added HCl/dioxane (4 M, 5 mL). The mixture was stirred at 15° C. for 6 hr. LCMS showed a peak (75%) with the mass of desired product and starting material was consumed. The reaction was concentrated under reduced pressure to afford a residue, which was triturated with EtOAc (20 mL) and MeCN (5 mL) to afford 1-(4-piperazin-1-ylphenyl) hexahydropyrimidine-2,4-dione (350 mg, 1.13 mmol, 84.34% yield, 100% purity, HCl) as yellow solid. MS(M+H)⁺=275.1.

¹H NMR (400 MHz, CD₃OD-d₄) δ=7.35-7.29 (m, 2H), 7.20-7.13 (m, 2H), 3.83 (t, J=6.8 Hz, 2H), 3.54-3.47 (m, 4H), 3.47-3.38 (m, 4H), 2.81 (t, J=6.8 Hz, 2H).

### Step 2. Synthesis of tert-butyl N-[4-[2-[4-[4-(2,4-dioxohexahydropyrimidin-1-yl)phenyl]piperazin-1-yl]ethyl]-1-piperidyl]carbamate (4)

To a solution of 1-(4-piperazin-1-ylphenyl) hexahydropyrimidine-2, 4-dione (250 mg, 911.35 μmol) and tert-butyl N-[4-(2-oxoethyl)-1-piperidyl]carbamate (441.67 mg, 1.82 mmol) in DCM (10 mL) was added TEA (276.66 mg, 2.73 mmol, 380.55 μL) and 4 Å MS (50 mg). After stirring at 15° C. for 0.5 hr, NaBH(OAc)₃ (579.46 mg, 2.73 mmol) was added and the resulting mixture was stirred at 15° C. for 16 hr. LCMS showed a peak (49%) with the mass of desired product. The reaction mixture was filtered and the filtrate was concentrated under reduced pressure to afford a residue. The residue was triturated with EtOAc (10 ml) to afford tert-butyl N-[4-[2-[4-[4-(2,4-dioxohexahydropyrimidin-1-yl)phenyl]piperazin-1-yl]ethyl]-1-piperid yl]carbamate (250 mg, 439.44 mol, 48.22% yield, 88% purity) as yellow solid. MS(M+H)⁺=501.3.

### Step 3. Synthesis of 1-[4-[4-[2-(1-amino-4-piperidyl)ethyl]piperazin-1-yl]phenyl]hexahydropyrimidine-2,4-dione (5)

To a solution of tert-butyl N-[4-[2-[4-[4-(2, 4-dioxohexahydropyrimidin-1-yl)phenyl]piperazin-1-yl]ethyl]-1-piperidyl]carbamate (250 mg, 499.37 μmol) in dioxane (4 mL) was added HCl/dioxane (4 M, 4 mL). The mixture was stirred at 15° C. for 2 hr. LCMS showed two peaks (78%) with the mass of desired product. The reaction mixture was concentrated under reduced pressure to afford 1-[4-[4-[2-(1-amino-4-piperidyl)ethyl]piperazin-1-yl]phenyl]hexahydropyrimidine-2, 4-dione (200 mg, 366.15 μmol, 73.32% yield, 80% purity, HCl) was obtained as yellow solid. MS(M+H)⁺=401.2.

### Step 4. Synthesis of N-(2-chlorophenyl)-4-[[2-[4-[[4-[2-[4-[4-(2,4-dioxohexahydropyrimidin-1-yl)phenyl]piperazin-1-yl]ethyl]-1-piperidyl]carbamoyl]anilino]-5-fluoro-pyrimidin-4-yl]amino]benzamide (Compound 54)

To a solution of 1-[4-[4-[2-(1-amino-4-piperidyl)ethyl]piperazin-1-yl]phenyl]hexahydropyrimidine-2,4-dione (150 mg, 374.52 μmol) and 4-[[4-[4-[(2-chlorophenyl)carbamoyl]anilino]-5-fluoro-pyrimidin-2-yl]amino]benzoic acid (107.38 mg, 224.71 μmol) in DMF (2 mL) were added HATU (213.60 mg, 561.77 mol) and DIPEA (145.21 mg, 1.12 mmol, 195.70 μL). The mixture was stirred at 15° C. for 16 hr. LCMS showed a peak (29%) with the mass of desired product. The reaction mixture was diluted with water (10 mL) and filtered to afford a residue. The residue was purified by prep-HPLC (column: Phenomenex luna C18 150*25 mm*10 um; mobile phase: [water (FA)-ACN]; gradient: 20%-40% B over 9 min) and lyophilized. The residue was further purified by prep-HPLC (column: Phenomenex luna C18 150*25 mm*10 um; mobile phase: [water (FA)-ACN]; gradient: 15%-45% B over 10 min) and lyophilized. The product was diluted with NaHCO₃ solution (15 mL) and extracted with EtOAc (10 mL×2). The combined organic phase was concentrated under reduced pressure and triturated with EtOAc/MeOH (3 mL, 20/1) to afford N-(2-chlorophenyl)-4-[[2-[4-[[4-[2-[4-[4-(2,4-dioxohexahydropyrimidin-1-yl)phenyl]piperazin-1-yl]ethyl]-1-piperidyl]carbamoyl]anilino]-5-fluoro-pyrimidin-4-yl]amino]benz amide (15.2 mg, 17.31 μmol, 4.62% yield, 92% purity) as white solid. MS(M+H)⁺=860.5.

¹H NMR (400 MHz, DMSO-d₆) δ=10.35-10.12 (m, 1H), 10.00-9.84 (m, 1H), 9.82-9.67 (m, 1H), 9.60 (s, 1H), 9.12 (s, 1H), 8.24 (br d, J=3.3 Hz, 1H), 8.08-7.92 (m, 4H), 7.79-7.73 (m, 2H), 7.72-7.62 (m, 3H), 7.56 (br d, J=7.9 Hz, 1H), 7.44-7.34 (m, 1H), 7.33-7.24 (m, 1H), 7.15 (br d, J=8.6 Hz, 2H), 6.93 (br d, J=8.6 Hz, 2H), 3.69 (br t, J=6.8 Hz, 2H), 3.20-3.05 (m, 4H), 3.03-2.92 (m, 3H), 2.74-2.65 (m, 6H), 2.38-2.30 (m, 2H), 1.74-1.63 (m, 2H), 1.50-1.35 (m, 2H), 1.35-1.18 (m, 4H).

### Example 55. Synthesis of N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (Compound 55)

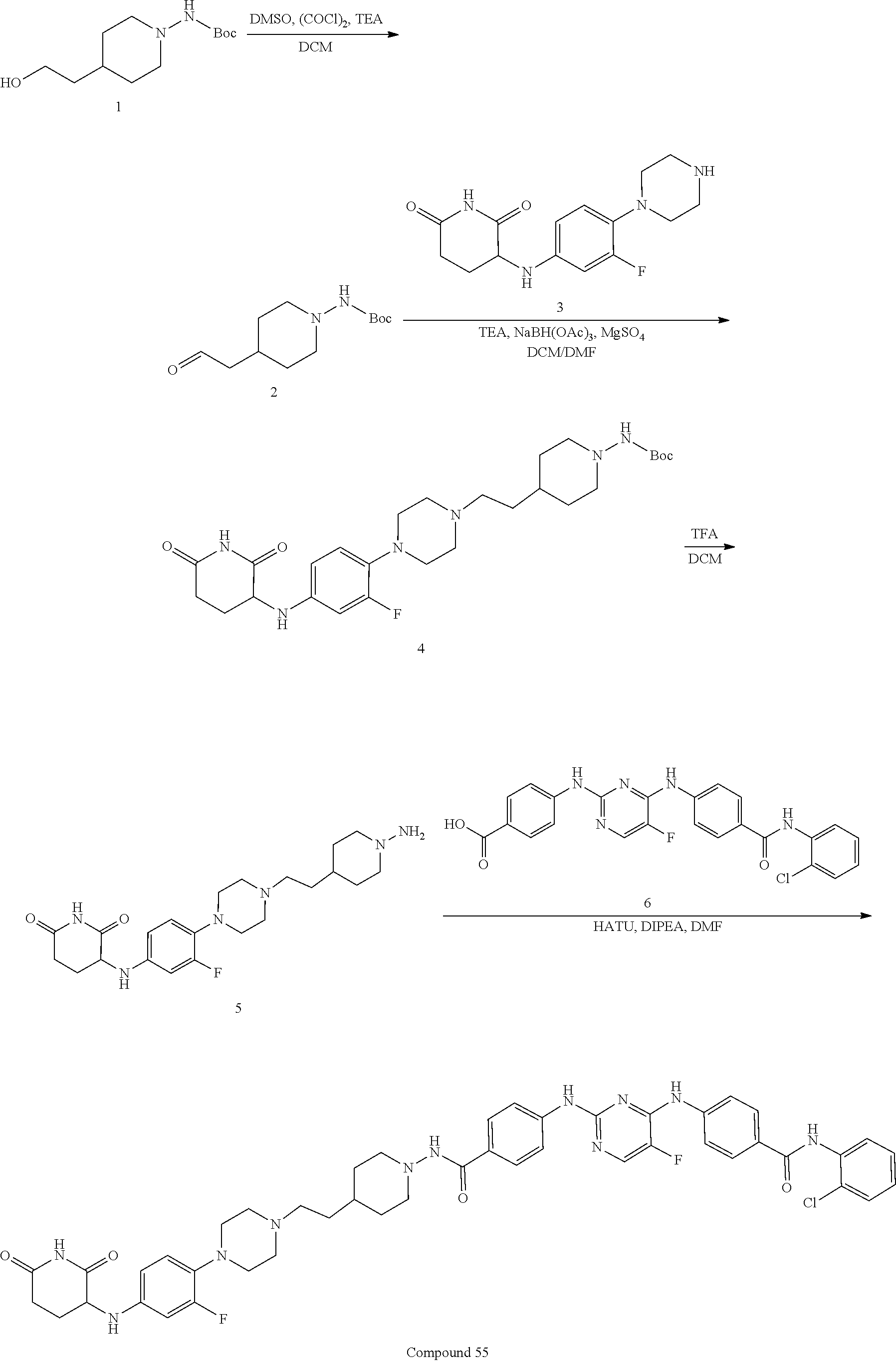

### Step 1. Synthesis of tert-butyl (4-(2-oxoethyl)piperidin-1-yl)carbamate (2)

To a solution of DMSO (1.50 g, 19.24 mmol, 1.50 mL) in DCM (10 mL) was slowly added (COCl)₂ (1.16 g, 9.14 mmol, 800 μL) at −70° C. and the mixture was stirred at −70° C. for 15 min. Then a solution of tert-butyl (4-(2-hydroxyethyl)piperidin-1-yl)carbamate (1 g, 4.09 mmol) in DCM (10 mL) was slowly added and the mixture was stirred at −70° C. for 20 min. TEA (2.04 g, 20.14 mmol, 2.80 mL) was added and the resulting mixture was stirred at −70° C. for 60 min. H NMR indicated the starting material was consumed completely and desired product was detected. The mixture was warmed to 20° C. and then quenched with water (20 mL). The mixture was extracted with DCM (10 mL×2), the combined organic layer was dried over MgSO₄ and filtered. The filtrate was concentrated in vacuum to afford tert-butyl (4-(2-oxoethyl)piperidin-1-yl) carbamate (1 g) as a yellow gum, which was used directly. MS(M+H)⁺=243.3.

### Step 2. Synthesis of tert-butyl (4-(2-(4-(4-((2,6-dioxopiperidin-3-yl)amino)-2-fluorophenyl)piperazin-1-yl)ethyl)piperidin-1-yl)carbamate (4)

To a mixture of 3-((3-fluoro-4-(piperazin-1-yl)phenyl)amino)piperidine-2,6-dione (300 mg, 875.16 μmol, HCl salt) in DCM (3 mL) were added TEA (442.78 mg, 4.38 mmol, 609.05 μL) and MgSO₄ (316.02 mg, 2.63 mmol), then followed by the addition of a solution of tert-butyl (4-(2-oxoethyl)piperidin-1-yl)carbamate (254.48 mg, 1.05 mmol) in DMF (3 mL). The mixture was stirred at 20° C. for 30 min. Then NaBH(OAc) 3 (556.44 mg, 2.63 mmol) was added at 0° C. and the mixture was stirred at 20° C. for 1 h. LCMS showed 3-((3-fluoro-4-(piperazin-1-yl)phenyl)amino)piperidine-2,6-dione was consumed completely and 30% of desired mass was detected. The mixture was quenched by addition H₂O (10 mL), and extracted with DCM (7 mL×3). The combined organic layers were washed with H₂O (10 mL×2) and concentrated in vacuum. The residue was triturated with MTBE (20 mL) for 10 minutes, the suspension was filtered and the filter cake was washed with MTBE (10 mL) and H₂O (5 mL), the filter cake was collected and dried in vacuum to afford tert-butyl (4-(2-(4-(4-((2,6-dioxopiperidin-3-yl)amino)-2-fluorophenyl)piperazin-1-yl)ethyl)piperidin-1-yl)carbamate (150 mg) as a gray solid. MS(M+H)⁺=533.3.

### Step 3. Synthesis of 3-((4-(4-(2-(1-aminopiperidin-4-yl)ethyl)piperazin-1-yl)-3-fluorophenyl) amino)piperidine-2,6-dione (5)

A mixture of tert-butyl (4-(2-(4-(4-((2,6-dioxopiperidin-3-yl)amino)-2-fluorophenyl)piperazin-1-yl)ethyl)piperidin-1-yl)carbamate (150 mg, 281.61 μmol) and TFA (1.54 g, 13.46 mmol, 1 mL) in DCM (3 mL) was stirred at 15° C. for 2 hours. LCMS showed the starting material was consumed completely and desired mass was detected. The mixture was concentrated in vacuum to afford 3-((4-(4-(2-(1-aminopiperidin-4-yl)ethyl)piperazin-1-yl)-3-fluorophenyl)amino)piperidine-2,6-dione (150 mg, TFA salt) as a brown oil, which was used directly. MS(M+H)⁺=433.2.

### Step 4. Synthesis of N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (Compound 55)

A mixture of 4-((4-((4-((2-chlorophenyl)carbamoyl)phenyl)amino)-5-fluoropyrimidin-2-yl)amino) benzoic acid (130 mg, 272.04 μmol), HATU (124.12 mg, 326.45 μmol) and DIPEA (351.59 mg, 2.72 mmol, 473.84 μL) in DMF (3 mL) was stirred at 15° C. for 15 minutes, then 3-((4-(4-(2-(1-aminopiperidin-4-yl)ethyl)piperazin-1-yl)-3-fluorophenyl)amino)piperidine-2,6-dione (150 mg, 274.45 mol, TFA salt) was added and the resulting mixture was stirred at 15° C. for 30 minutes. LCMS showed 3-((4-(4-(2-(1-aminopiperidin-4-yl)ethyl)piperazin-1-yl)-3-fluorophenyl)amino)piperidine-2,6-dione was consumed completely and 63% of desired mass was detected. The mixture was poured into a mixture (EtOAc/THF/H₂O=2/1/3, 60 mL) and the layers were separated. The aqueous phase was extracted with a mixture (EtOAc/THF/H₂O=2/1, 30 mL×4). The combined organic layers were washed with brine (20 mL×2), dried over MgSO₄, filtered and concentrated in vacuum. The residue was triturated with a mixture (EtOAc/THF=20/1, 15 mL) for 30 minutes, the suspension was filtered and the filter cake was washed with a mixture (EtOAc/THF=20/1, 6 mL), the filter cake was collected and dried in vacuum. The residue was diluted with a mixture (ACN/THF/H₂O=2/1/10, 26 mL) and freeze-dried. The residue was triturated with a mixture (ACN/THF=1/1, 4 mL) for 10 minutes, the suspension was filtered and the filter cake was washed with a mixture (ACN/THF=1/1, 6 mL). The filter cake was collected and dried. The residue was diluted with a mixture (ACN/THF/H₂O=1/1/5, 21 mL) and lyophilized to afford N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (41.7 mg, 42.52 μmol, 15.63% yield, 91% purity) as an off-white solid. MS(M+H)⁺=892.2.

¹H NMR (400 MHz, DMSO-d₆) δ=10.80 (s, 1H), 9.96 (s, 1H), 9.70 (s, 1H), 9.61 (s, 1H), 9.12 (s, 1H), 8.24 (s, 1H), 8.12-7.93 (m, 4H), 7.82-7.74 (m, 2H), 7.73-7.67 (m, 2H), 7.65 (br d, J=8.2 Hz, 1H), 7.57 (br d, J=7.9 Hz, 1H), 7.44-7.35 (m, 1H), 7.34-7.24 (m, 1H), 6.90-6.80 (m, 1H), 6.58-6.48 (m, 1H), 6.47-6.38 (m, 1H), 5.92-5.78 (m, 1H), 4.33-4.20 (m, 1H), 3.10-2.83 (m, 7H), 2.82-2.65 (m, 5H), 2.62-2.58 (m, 2H), 2.56-2.53 (m, 1H), 2.12-2.05 (m, 1H), 1.92-1.78 (m, 1H), 1.76-1.61 (m, 2H), 1.59-1.41 (m, 2H), 1.40-1.13 (m, 4H).

### Example 56. Synthesis of N-(2-chlorophenyl)-4-[[2-[4-[[4-[2-[4-[5-(2,6-dioxo-3-piperidyl)-2-pyridyl]piperazin-1-yl]ethyl]-1-piperidyl]carbamoyl]anilino]-5-fluoro-pyrimidin-4-yl]amino]benzamide (Compound 56)

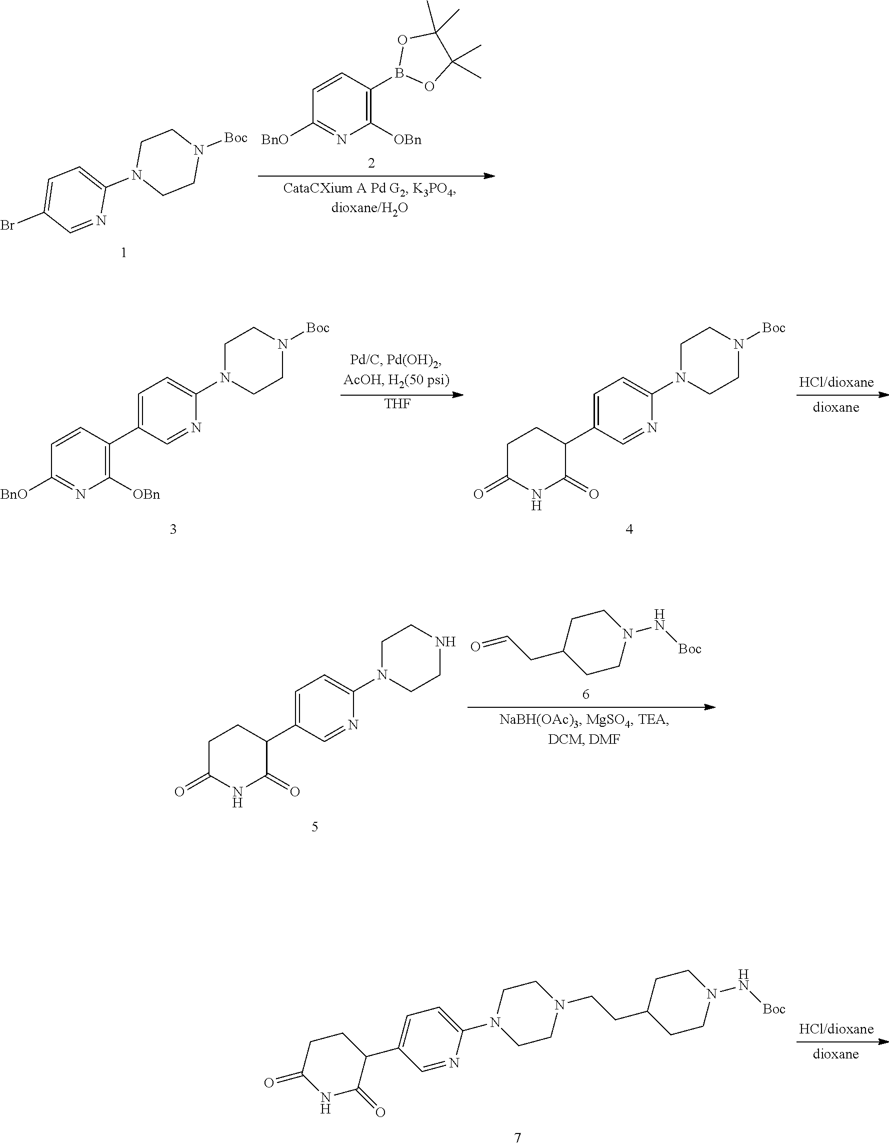

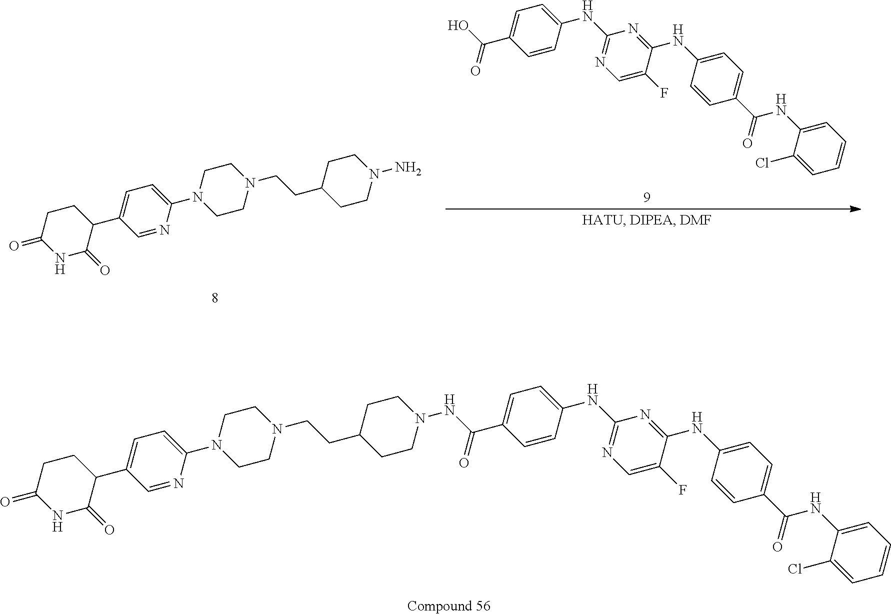 [SMILES: NN1CCC(CCN2CCN(c3ccc(C4CCC(=O)NC4=O)cn3)CC2)CC1 ; O=C(O)c1ccc(Nc2ncc(F)c(Nc3ccc(C(=O)Nc4ccccc4Cl)cc3)n2)cc1 ; O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)nc2)C(=O)N1]

### Step 1. Synthesis of tert-butyl 4-[5-(2,6-dibenzyloxy-3-pyridyl)-2-pyridyl]piperazine-1-carboxylate (3)

A mixture of tert-butyl 4-(5-bromo-2-pyridyl)piperazine-1-carboxylate (500 mg, 1.46 mmol), 2,6-dibenzyloxy-3-(4,4,5,5-tetramethyl-1,3,2-dioxaborolan-2-yl)pyridine (609.68 mg, 1.46 mmol), CataCXium A Pd G2 (48.84 mg, 73.05 μmol), K₃PO₄ (930.36 mg, 4.38 mmol) in dioxane (9 mL) and H₂O (3 mL) was degassed and purged with N₂ for 3 times, and then the mixture was stirred at 80° C. for 16 hr under N₂ atmosphere. LCMS showed a peak (60%) with the mass of desired product and a peak (30%) with the mass of the mass of tert-butyl 4-(5-bromo-2-pyridyl)piperazine-1-carboxylate. To the mixture was added 2,6-dibenzyloxy-3-(4,4,5,5-tetramethyl-1,3,2-dioxaborolan-2-yl)pyridine (609.68 mg, 1.46 mmol) and [2-(2-aminophenyl)phenyl]-chloro-palladium; bis (1-adamantyl)-butyl-phosphane (48.84 mg, 73.05 μmol). The mixture was degassed and purged with N₂ for 3 times, and then the mixture was stirred at 80° C. for 16 hr under N₂ atmosphere. LCMS showed a peak (42%) with the mass of desired product and tert-butyl 4-(5-bromo-2-pyridyl)piperazine-1-carboxylate was consumed. The reaction mixture was diluted with water (30 mL) and extracted with EtOAc (20 mL×3). The combined organic phase was washed with brine, dried over Na₂SO₄ and concentrated under reduced pressure to afford a residue, which was purified by flash silica gel chromatography (12 g SepaFlash® Silica Flash Column, Eluent of 0~50% EtOAc/Petroleum ether, gradient @ 60 mL/min) to afford tert-butyl 4-[5-(2, 6-dibenzyloxy-3-pyridyl)-2-pyridyl]piperazine-1-carboxylate (800 mg, 1.36 mmol, 93.13% yield, 94% purity) as yellow solid. MS(M+H)⁺=553.3.

¹H NMR (400 MHz, DMSO-d₆) δ=8.32 (d, J=2.3 Hz, 1H), 7.78-7.70 (m, 2H), 7.46-7.27 (m, 10H), 6.87 (d, J=8.9 Hz, 1H), 6.54 (d, J=8.1 Hz, 1H), 5.40 (s, 2H), 5.37 (s, 2H), 3.54-3.46 (m, 4H), 3.46-3.38 (m, 4H), 1.43 (s, 9H).

### Step 2. Synthesis of tert-butyl 4-[5-(2,6-dioxo-3-piperidyl)-2-pyridyl]piperazine-1-carboxylate (4)

To a mixture of Pd/C (154.05 mg, 144.75 mol, 10% purity) and Pd(OH)₂ (101.64 mg, 144.75 μmol, 20% purity) in THF (10 mL) was added tert-butyl 4-[5-(2, 6-dibenzyloxy-3-pyridyl)-2-pyridyl]piperazine-1-carboxylate (800 mg, 1.45 mmol) and AcOH (4.35 mg, 72.38 mol, 4.14 μL) under N₂ atmosphere. The suspension was degassed and purged with H₂ for 3 times. The mixture was stirred under H₂ (50 Psi) at 30° C. for 48 hr. LCMS showed a peak (15%) with the mass of desired product and tert-butyl 4-[5-(2,6-dibenzyloxy-3-pyridyl)-2-pyridyl]piperazine-1-carboxylate remained. The reaction mixture was filtered through celite pad and concentrated under reduced pressure to afford a residue. To a mixture of Pd/C (308.09 mg, 289.51 μmol, 10% purity) and Pd(OH)₂ (203.29 mg, 289.51 μmol, 20% purity) in THF (10 mL) was added the residue and AcOH (86.93 mg, 1.45 mmol, 82.87 μL) under N₂ atmosphere. The suspension was degassed and purged with H₂ for 3 times. The mixture was stirred under H₂ (50 Psi) at 50° C. for 48 hr. LCMS showed a peak (67%) with the mass of desired product and tert-butyl 4-[5-(2,6-dibenzyloxy-3-pyridyl)-2-pyridyl]piperazine-1-carboxylate was consumed. The reaction mixture was filtered through celite pad and washed with EtOAc. The filtrate was concentrated under reduced pressure to afford tert-butyl 4-[5-(2,6-dioxo-3-piperidyl)-2-pyridyl]piperazine-1-carboxylate (540 mg, crude) was obtained as black oil. MS(M+H)⁺=375.1.

### Step 3. Synthesis of 3-(6-piperazin-1-yl-3-pyridyl)piperidine-2,6-dione (5)

To a solution of tert-butyl 4-[5-(2,6-dioxo-3-piperidyl)-2-pyridyl]piperazine-1-carboxylate (540 mg, 1.44 mmol) in dioxane (10 mL) was added HCl/dioxane (4 M, 10 mL). The mixture was stirred at 15° C. for 3 hr. LCMS showed a peak (82%) with the mass of desired product. The reaction mixture was concentrated under reduced pressure to afford a residue. The residue was triturated with 30 mL of EtOAc to afford 3-(6-piperazin-1-yl-3-pyridyl)piperidine-2, 6-dione (400 mg, 1.11 mmol, 76.75% yield, 86% purity, HCl) was obtained as purple solid. MS (M+H)⁺=275.1.

### Step 4. Synthesis of tert-butyl tert-butyl N-[4-[2-[4-[5-(2,6-dioxo-3-piperidyl)-2-pyridyl]piperazin-1-yl]ethyl]-1-piperidyl]carbamate (7)

To a solution of 3-(6-piperazin-1-yl-3-pyridyl)piperidine-2, 6-dione (300 mg, 1.09 mmol) and tert-butyl N-[4-(2-oxoethyl)-1-piperidyl]carbamate (397.50 mg, 1.64 mmol) in DCM (3 mL) and DMF (3 mL) was added TEA (553.31 mg, 5.47 mmol, 761.09 μL) and MgSO₄ (394.91 mg, 3.28 mmol). After stirring at 15° C. for 0.5 hr, NaBH(OAc)₃ (695.35 mg, 3.28 mmol) was added in portions and the resulting mixture was stirred at 15° C. for 16 hr. LCMS showed a peak (31%) with the mass of desired product. The reaction mixture was diluted with water (20 mL) and extracted with DCM (20 mL×3). The combined organic phase was dried over Na₂SO₄ and concentrated under reduced pressure to afford a residue, which was triturated with MTBE (20 mL) to afford tert-butyl N-[4-[2-[4-[5-(2,6-dioxo-3-piperidyl)-2-pyridyl]piperazin-1-yl]ethyl]-1-piperidyl]carbamate (220 mg, 373.53 μmol, 34.16% yield, 85% purity) as yellow solid. MS(M+H)⁺=501.3.

### Step 5. Synthesis of 3-[6-[4-[2-(1-amino-4-piperidyl)ethyl]piperazin-1-yl]-3-pyridyl]piperidine-2,6-dione

To a solution of tert-butyl N-[4-[2-[4-[5-(2,6-dioxo-3-piperidyl)-2-pyridyl]piperazin-1-yl]ethyl]-1-piperidyl]carbamate (220 mg, 439.44 μmol) in dioxane (2 mL) was added HCl/dioxane (4 M, 2 mL). The mixture was stirred at 20° C. for 2 hr. LCMS showed a peak (67%) with the mass of desired product. The reaction mixture was concentrated under reduced pressure to afford 3-[6-[4-[2-(1-amino-4-piperidyl)ethyl]piperazin-1-yl]-3-pyridyl]piperidine-2,6-dione (190 mg, 286.97 mol, 65.30% yield, 66% purity, HCl) as yellow solid. MS(M+H)⁺=401.3.

### Step 6. Synthesis of N-(2-chlorophenyl)-4-[[2-[4-[[4-[2-[4-[5-(2,6-dioxo-3-piperidyl)-2-pyridyl]piperazin-1-yl]ethyl]-1-piperidyl]carbamoyl]anilino]-5-fluoro-pyrimidin-4-yl]amino]benzamide (Compound 56)

To a solution of 3-[6-[4-[2-(1-amino-4-piperidyl) ethyl]piperazin-1-yl]-3-pyridyl]piperidine-2, 6-dione (170 mg, 424.45 μmol) and 4-[[4-[4-[(2-chlorophenyl)carbamoyl]anilino]-5-fluoro-pyrimidin-2-yl]amino]benzoic acid (202.83 mg, 424.45 μmol) in DMF (3 mL) were added HATU (193.67 mg, 509.34 mol) and DIPEA (164.57 mg, 1.27 mmol, 221.80 μL). The mixture was stirred at 20° C. for 1 hr. LCMS showed a peak (25%) with the mass of desired product. The reaction mixture was diluted with water (20 mL) and extracted with EtOAc (10 mL×3). The combined organic layers were washed with brine, dried over Na₂SO₄ and concentrated. The crude product was purified by prep-HPLC (column: Phenomenex luna C18 150*25 mm*10 um; mobile phase: [water (TFA)-ACN]; gradient: 21%-51% B over 10 min) and lyophilized to afford N-(2-chlorophenyl)-4-[[2-[4-[[4-[2-[4-[5-(2,6-dioxo-3-piperidyl)-2-pyridyl]piperazin-1-yl]ethyl]-1-piperidyl]carbamoyl]anilino]-5-fluoro-pyrimidin-4-yl]amino]benzamide (12.9 mg, 8.53 μmol, 2.01% yield, 87% purity, 4TFA) as brown solid. MS(M+H)⁺=860.3.

¹H NMR (400 MHz, DMSO-d₆) δ=10.85 (s, 1H), 9.96 (s, 2H), 9.79 (br s, 1H), 9.70 (s, 1H), 8.26 (br d, J=3.1 Hz, 1H), 8.10-8.00 (m, 5H), 7.84-7.70 (m, 4H), 7.64 (br d, J=8.3 Hz, 1H), 7.60-7.49 (m, 2H), 7.40 (br t, J=7.8 Hz, 1H), 7.35-7.24 (m, 1H), 7.03-6.93 (m, 1H), 4.46-4.31 (m, 2H), 3.80 (br dd, J=4.5, 12.5 Hz, 1H), 3.67-3.52 (m, 2H), 3.26-2.98 (m, 8H), 2.95-2.82 (m, 3H), 2.78-2.63 (m, 1H), 2.28-2.14 (m, 1H), 2.04-1.89 (m, 1H), 1.85-1.58 (m, 4H), 1.49-1.30 (m, 3H).

### Example 57. Synthesis of 4-((4-((2-chlorophenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (Compound 57)

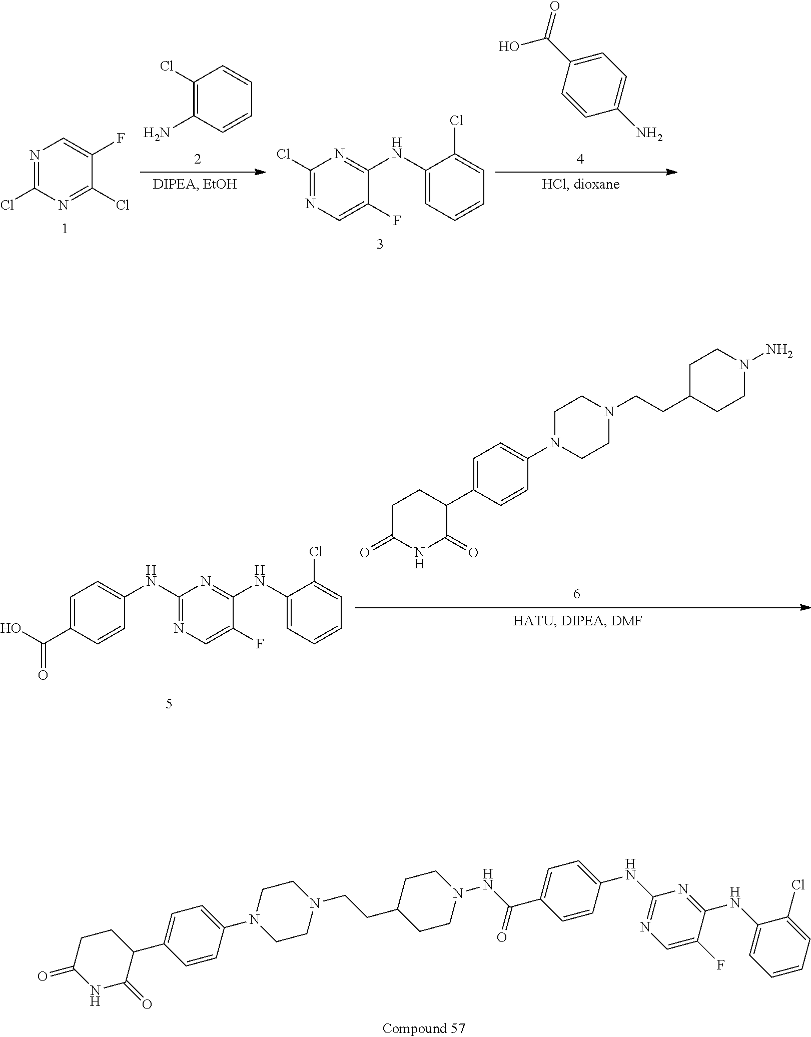

### Step 1. Synthesis of 2-chloro-N-(2-chlorophenyl)-5-fluoropyrimidin-4-amine (3)

To a solution of 2,4-dichloro-5-fluoropyrimidine (2 g, 11.98 mmol) and 2-chloroaniline (1.53 g, 11.98 mmol, 1.26 mL) in EtOH (20 mL) was added DIPEA (4.64 g, 35.93 mmol, 6.26 mL). The mixture was stirred at 60° C. for 16 hr. LCMS showed 2,4-dichloro-5-fluoropyrimidine was consumed completely. The reaction mixture was diluted with H₂O (40 mL) at 20° C., and then extracted with EtOAc (20 mL×2). The combined organic layers were dried over Na₂SO₄, filtered and concentrated under reduced pressure to give a residue, which was purified by flash silica gel chromatography (ISCO®; 40 g SepaFlash® Silica Flash Column, Eluent of 0-15% EtOAc/Petroleum ether, gradient @ 100 mL/min) to afford 2-chloro-N-(2-chlorophenyl)-5-fluoropyrimidin-4-amine (140 mg, 542.47 μmol, 4.53% yield) as a yellow solid. MS(M+H)⁺=258.0.

### Step 2. Synthesis of 4-((4-((2-chlorophenyl)amino)-5-fluoropyrimidin-2-yl)amino)benzoic acid (5)

To a solution of 2-chloro-N-(2-chlorophenyl)-5-fluoropyrimidin-4-amine (140 mg, 542.47 μmol) and 4-aminobenzoic acid (74.39 mg, 542.47 μmol) in dioxane (1.5 mL) was added HCl (12 M, 38.78 μL). The mixture was stirred at 80° C. for 16 hr. LCMS showed 83% of 2-chloro-N-(2-chlorophenyl)-5-fluoropyrimidin-4-amine remained and a peak of 13% was desired mass. Another portion of 4-aminobenzoic acid (74.39 mg, 542.47 μmol) and HCl (12 M, 135.62 μL) were added and the mixture was stirred at 80° C. for 16 hr. LCMS showed 2-chloro-N-(2-chlorophenyl)-5-fluoropyrimidin-4-amine was consumed completely. The reaction mixture was diluted with H₂O (5 mL) and EA (10 mL). The mixture was filtered. The filter cake was collected and dried to afford 4-((4-((2-chlorophenyl)amino)-5-fluoropyrimidin-2-yl)amino)benzoic acid (56 mg, 156.10 mol, 28.78% yield) as a white solid. MS(M+H)⁺=359.0.

### Step 3. Synthesis of 4-((4-((2-chlorophenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (Compound 57)

To a solution of 4-((4-((2-chlorophenyl)amino)-5-fluoropyrimidin-2-yl)amino)benzoic acid (56 mg, 156.10 μmol) in DMF (1 mL) were added HATU (89.03 mg, 234.14 μmol) and DIPEA (100.87 mg, 780.48 μmol, 135.95 μL). After stirring at 25° C. for 0.5 hr, 3-(4-(4-(2-(1-aminopiperidin-4-yl)ethyl)piperazin-1-yl)phenyl)piperidine-2,6-dione (140 mg, 272.61 mol, TFA) was added and the mixture was stirred at 25° C. for 16 hr. LCMS showed 3-(4-(4-(2-(1-aminopiperidin-4-yl)ethyl)piperazin-1-yl)phenyl)piperidine-2,6-dione was consumed completely. The reaction mixture was diluted with H₂O (10 mL) at 25° C., and then extracted with EtOAc (10 mL×2). The combined organic layers were washed with brine (10 mL), dried over Na₂SO₄, filtered and concentrated under reduced pressure to give a residue. The residue was purified by flash silica gel chromatography (ISCO®; 12 g SepaFlash® Silica Flash Column, Eluent of 0~20% MeOH/EtOAc @ 60 mL/min). The impure product was further purified by prep-HPLC (column: Waters Xbridge C18 150*50 mm*10 um; mobile phase: [water (NH₄HCO₃)-ACN]; gradient: 27%-57% B over 10 min) and lyophilized to afford 4-((4-((2-chlorophenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (12 mg, 15.89 mol, 10.18% yield, 98% purity) as a white solid. MS(M+H)⁺=740.3.

¹H NMR (400 MHz, DMSO-d₆) δ=10.88-10.70 (m, 1H), 9.41 (s, 1H), 9.35-9.17 (m, 1H), 9.10 (s, 1H), 8.14 (d, J=3.5 Hz, 1H), 7.67-7.57 (m, 2H), 7.56-7.41 (m, 5H), 7.40-7.33 (m, 1H), 7.05 (d, J=8.4 Hz, 2H), 6.89 (br d, J=8.8 Hz, 2H), 3.78-3.67 (m, 1H), 3.15-3.05 (m, 4H), 3.02-2.95 (m, 2H), 2.78-2.65 (m, 3H), 2.65-2.55 (m, 2H), 2.50-2.45 (m, 2H), 2.39-2.29 (m, 3H), 2.19-2.06 (m, 1H), 2.05-1.95 (m, 1H), 1.75-1.65 (m, 2H), 1.48-1.36 (m, 2H), 1.35-1.20 (m, 3H).

### Example 58. Synthesis of N-(4-(2-(4-(4-(2,6-dioxopiperidin-3-yl)phenyl)piperazin-1-yl)ethyl)piperidin-1-yl)-4-((5-fluoro-4-(phenylamino)pyrimidin-2-yl)amino)benzamide (Compound 58)

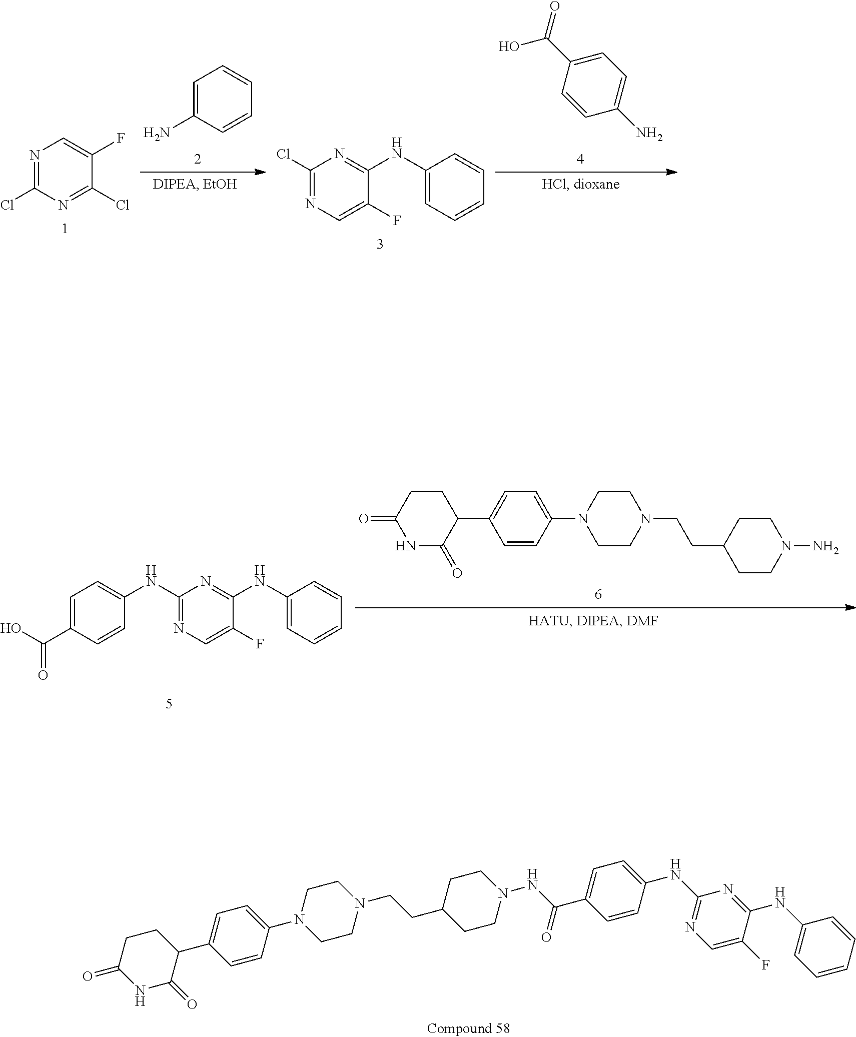

### Step 1. Synthesis of 2-chloro-5-fluoro-N-phenylpyrimidin-4-amine (3)

To a solution of 2,4-dichloro-5-fluoropyrimidine (1 g, 5.99 mmol) and aniline (557.75 mg, 5.99 mmol, 545.74 μL) in EtOH (10 mL) was added DIPEA (2.32 g, 17.97 mmol, 3.13 mL). The mixture was stirred at 80° C. for 16 hr. LCMS showed 2,4-dichloro-5-fluoropyrimidine was consumed completely and desired mass was detected. The reaction mixture was diluted with H₂O (40 mL) at 20° C., and then extracted with EtOAc (20 mL×2). The combined organic layers were dried over Na₂SO₄, filtered and concentrated under reduced pressure. The residue was purified by flash silica gel chromatography (ISCO®; 20 g SepaFlash® Silica Flash Column, Eluent of 0~15% EtOAc/Petroleum ether, gradient @ 100 mL/min) to afford 2-chloro-5-fluoro-N-phenylpyrimidin-4-amine (1.2 g, 5.31 mmol, 88.70% yield, 99% purity) as a white solid. MS(M+H)⁺=224.1.

### Step 2. Synthesis of 4-((5-fluoro-4-(phenylamino)pyrimidin-2-yl)amino)benzoic acid (5)

To a solution of 2-chloro-5-fluoro-N-phenylpyrimidin-4-amine (500 mg, 2.24 mmol) and 4-aminobenzoic acid (306.61 mg, 2.24 mmol) in EtOH (5 mL) and was added HCl (12 M, 159.84 μL). The mixture was stirred at 80° C. for 16 hr. LCMS showed 2-chloro-5-fluoro-N-phenylpyrimidin-4-amine was consumed completely and desired mass was detected. The reaction mixture was diluted with H₂O (10 mL) and EtOAc (20 mL). The mixture was filtered. The filter cake was collected and dried to afford the 4-((5-fluoro-4-(phenylamino)pyrimidin-2-yl)amino)benzoic acid (510 mg, 1.13 mmol, 50.64% yield, 72% purity) as a white solid. MS(M+H)⁺=325.3.

### Step 3. Synthesis of N-(4-(2-(4-(4-(2,6-dioxopiperidin-3-yl)phenyl)piperazin-1-yl)ethyl)piperidin-1-yl)-4-((5-fluoro-4-(phenylamino)pyrimidin-2-yl)amino)benzamide (Compound 58)

To a solution of 4-((5-fluoro-4-(phenylamino)pyrimidin-2-yl)amino)benzoic acid (100 mg, 308.35 μmol) in DMF (1 mL) were added HATU (175.86 mg, 462.52 μmol) and DIPEA (199.26 mg, 1.54 mmol, 268.54 μL). After stirring at 25° C. for 0.5 hr, 3-(4-(4-(2-(1-aminopiperidin-4-yl)ethyl)piperazin-1-yl)phenyl)piperidine-2,6-dione (237.53 mg, 462.52 μmol, TFA) was added and the resulting mixture was stirred at 25° C. for 2 hr. LCMS showed 4-((5-fluoro-4-(phenylamino)pyrimidin-2-yl)amino)benzoic acid was consumed completely and desired mass was detected. The reaction mixture was diluted with H₂O (20 mL) at 25° C. and extracted with EtOAc (10 mL×2). The combined organic layers were washed with brine (10 mL), dried over Na₂SO₄, filtered and concentrated under reduced pressure. The residue was purified by flash silica gel chromatography (ISCO®; 12 g SepaFlash® Silica Flash Column, Eluent of 0~20% MeOH/EtOAc @ 50 mL/min). The impure product was further purified by prep-HPLC (column: Waters Xbridge C18 150*50 mm*10 um; mobile phase: [water (NH₄HCO₃)-ACN]; gradient: 24%-54% B over 10 min) and lyophilized to afford N-(4-(2-(4-(4-(2,6-dioxopiperidin-3-yl)phenyl)piperazin-1-yl)ethyl)piperidin-1-yl)-4-((5-fluoro-4-(phenylamino)pyrimidin-2-yl)amino)benzamide (8.2 mg, 11.39 μmol, 3.69% yield, 98% purity) as a white solid. MS(M+H)⁺=706.5.

¹H NMR (400 MHz, DMSO-d₆) δ=10.84-10.72 (m, 1H), 9.50 (s, 1H), 9.43 (s, 1H), 9.14 (s, 1H), 8.16 (d, J=3.7 Hz, 1H), 7.79 (br d, J=7.9 Hz, 2H), 7.74 (br d, J=8.8 Hz, 2H), 7.64 (br d, J=8.3 Hz, 2H), 7.43-7.31 (m, 2H), 7.12 (br t, J=7.3 Hz, 1H), 7.04 (br d, J=8.4 Hz, 2H), 6.89 (br d, J=8.4 Hz, 2H), 3.78-3.68 (m, 1H), 3.17-3.04 (m, 4H), 3.00-2.90 (m, 2H), 2.79-2.69 (m, 2H), 2.67-2.64 (m, 2H), 2.62-2.58 (m, 1H), 2.50-2.44 (m, 2H), 2.38-2.29 (m, 3H), 2.20-2.07 (m, 1H), 2.06-1.94 (m, 1H), 1.77-1.63 (m, 2H), 1.49-1.37 (m, 2H), 1.35-1.21 (m, 3H).

### Example 59. Synthesis of N-(4-(2-(4-(4-(2,6-dioxopiperidin-3-yl)phenyl)piperazin-1-yl)ethyl)piperidin-1-yl)-4-((5-fluoro-4-((5-methyl-1H-pyrazol-3-yl)amino)pyrimidin-2-yl)amino)benzamide (Compound 59)

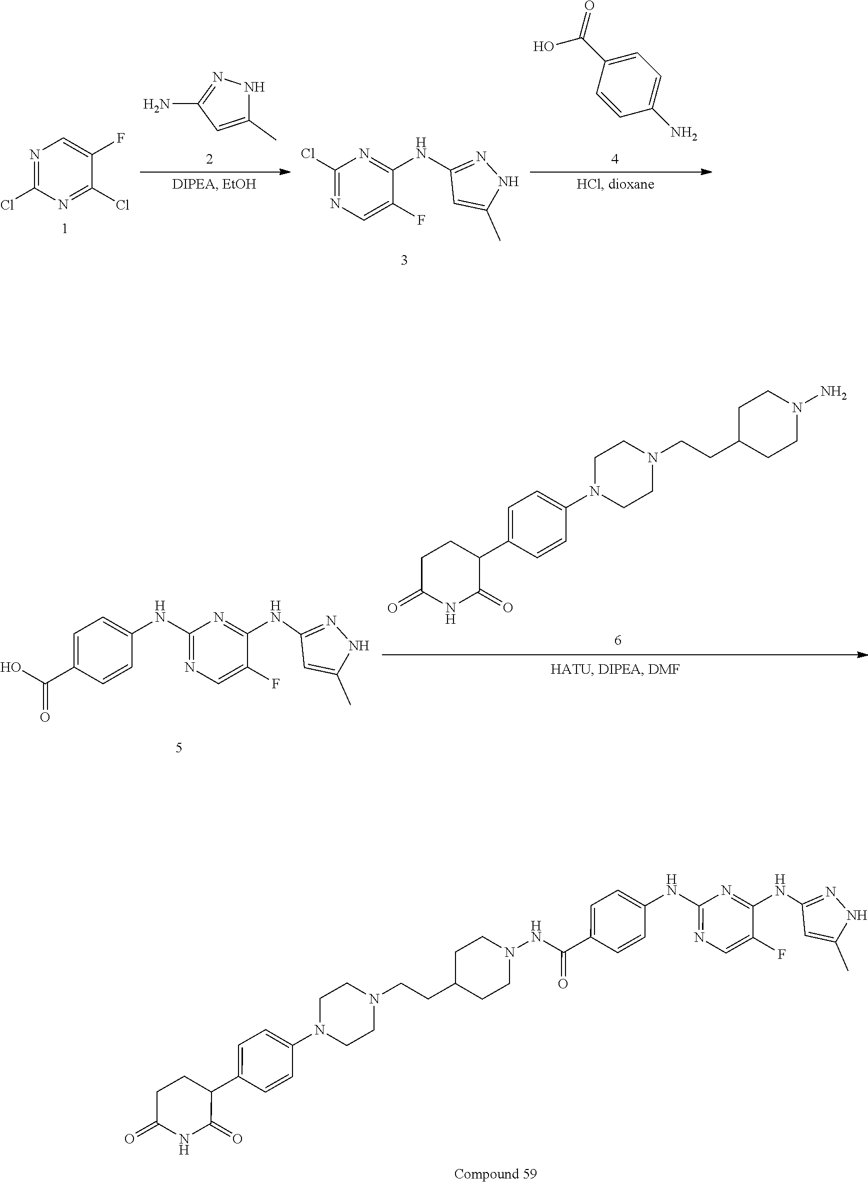

### Step 1. Synthesis of 2-chloro-5-fluoro-N-(5-methyl-1H-pyrazol-3-yl)pyrimidin-4-amine (3)

To a solution of 2,4-dichloro-5-fluoropyrimidine (1 g, 5.99 mmol) and 5-methyl-1H-pyrazol-3-amine (581.66 mg, 5.99 mmol) in EtOH (10 mL) was added DIPEA (2.32 g, 17.97 mmol, 3.13 mL). The mixture was stirred at 80° C. for 16 hr. LCMS showed 2,4-dichloro-5-fluoropyrimidine was consumed completely and desired mass was detected. The reaction mixture was diluted with H₂O (10 mL) and EtOAc (20 mL). The mixture was filtered. The filter cake was collected and dried to afford 2-chloro-5-fluoro-N-(5-methyl-1H-pyrazol-3-yl)pyrimidin-4-amine (800 mg, 3.51 mmol, 58.68% yield) as a white solid. MS(M+H)⁺=228.0.

### Step 2. Synthesis of 4-((5-fluoro-4-((5-methyl-1H-pyrazol-3-yl)amino)pyrimidin-2-yl)amino) benzoic acid

To a solution of 4 2-chloro-5-fluoro-N-(5-methyl-1H-pyrazol-3-yl)pyrimidin-4-amine (200 mg, 878.64 μmol) and 4-aminobenzoic acid (120.49 mg, 878.64 μmol) in iPrOH (2 mL) was added HCl (12 M, 94.22 μL). The mixture was stirred at 80° C. for 16 hr. LCMS showed 2-chloro-5-fluoro-N-(5-methyl-1H-pyrazol-3-yl)pyrimidin-4-amine was consumed completely and desired mass was detected. The reaction mixture was diluted with H₂O (5 mL) and EtOAc (10 mL). The mixture was filtered. The filter cake was collected and dried to afford 4-((5-fluoro-4-((5-methyl-1H-pyrazol-3-yl)amino)pyrimidin-2-yl)amino)benzoic acid (260 mg, 791.96 mol, 90.13% yield) as a white solid. MS(M+H)⁺=329.2.

### Step 3. Synthesis of N-(4-(2-(4-(4-(2,6-dioxopiperidin-3-yl)phenyl)piperazin-1-yl)ethyl)piperidin-1-yl)-4-((5-fluoro-4-((5-methyl-1H-pyrazol-3-yl)amino)pyrimidin-2-yl)amino)benzamide (Compound 59)

To a solution of 4-((5-fluoro-4-((5-methyl-1H-pyrazol-3-yl)amino)pyrimidin-2-yl)amino)benzoic acid (100 mg, 304.60 μmol) in DMF (1 mL) were added HATU (173.73 mg, 456.90 μmol) and DIPEA (196.84 mg, 1.52 mmol, 265.28 μL). After stirring at 25° C. for 0.5 hr, 3-(4-(4-(2-(1-aminopiperidin-4-yl)ethyl)piperazin-1-yl)phenyl)piperidine-2,6-dione (234.64 mg, 456.90 μmol, TFA) was added and the resulting mixture was stirred at 25° C. for 1 hr. LCMS showed 4-((5-fluoro-4-((5-methyl-1H-pyrazol-3-yl)amino)pyrimidin-2-yl)amino)benzoic acid was consumed completely and desired mass was detected. The reaction mixture was diluted H₂O (20 mL) and extracted with EtOAc (10 mL×2). The combined organic layers were washed with brine (20 mL), dried over Na₂SO₄, filtered and concentrated under reduced pressure. The residue was purified by flash silica gel chromatography (ISCO®; 12 g SepaFlash® Silica Flash Column, Eluent of 0~20% MeOH/EtOAc @ 50 mL/min). The impure product was purified by prep-HPLC (column: Waters Xbridge 150*25 mm 10 um; mobile phase: [water (NH₄HCO₃)-ACN]; gradient: 27%-47% B over 10 min) and lyophilized to afford N-(4-(2-(4-(4-(2,6-dioxopiperidin-3-yl)phenyl)piperazin-1-yl)ethyl)piperidin-1-yl)-4-((5-fluoro-4-((5-methyl-1H-pyrazol-3-yl)amino)pyrimidin-2-yl)amino)benzamide (2 mg, 2.76 μmol, 9.07e-1% yield, 98% purity) as a white solid. MS(M+H)⁺=710.3.

¹H NMR (400 MHz, DMSO-d₆) δ=12.12 (s, 1H), 10.77 (s, 1H), 9.71-9.55 (m, 1H), 9.48-9.32 (m, 1H), 9.19-9.08 (m, 1H), 8.06 (br d, J=2.2 Hz, 1H), 7.84-7.57 (m, 4H), 7.04 (br d, J=8.2 Hz, 2H), 6.94-6.83 (m, 2H), 6.56-6.43 (m, 1H), 3.77-3.67 (m, 1H), 3.2-3.05 (m, 4H), 3.00-2.90 (m, 2H), 2.78-2.69 (m, 2H), 2.63-2.56 (m, 4H), 2.46-2.42 (m, 3H), 2.40-2.33 (m, 2H), 2.32-2.27 (m, 2H), 2.18-2.10 (m, 1H), 2.05-1.94 (m, 1H), 1.77-1.63 (m, 2H), 1.49-1.37 (m, 2H), 1.33-1.22 (m, 3H).

### Example 60. Synthesis of N-(2-chlorophenyl)-4-((2-((4-((4-(7-(4-(2,6-dioxopiperidin-3-yl)phenyl)-7-azaspiro[3.5]nonan-2-yl)piperidin-1-yl)carbamoyl)phenyl)amino)-5-fluoropyrimidin-4-yl)amino)benzamide (Compound 60)

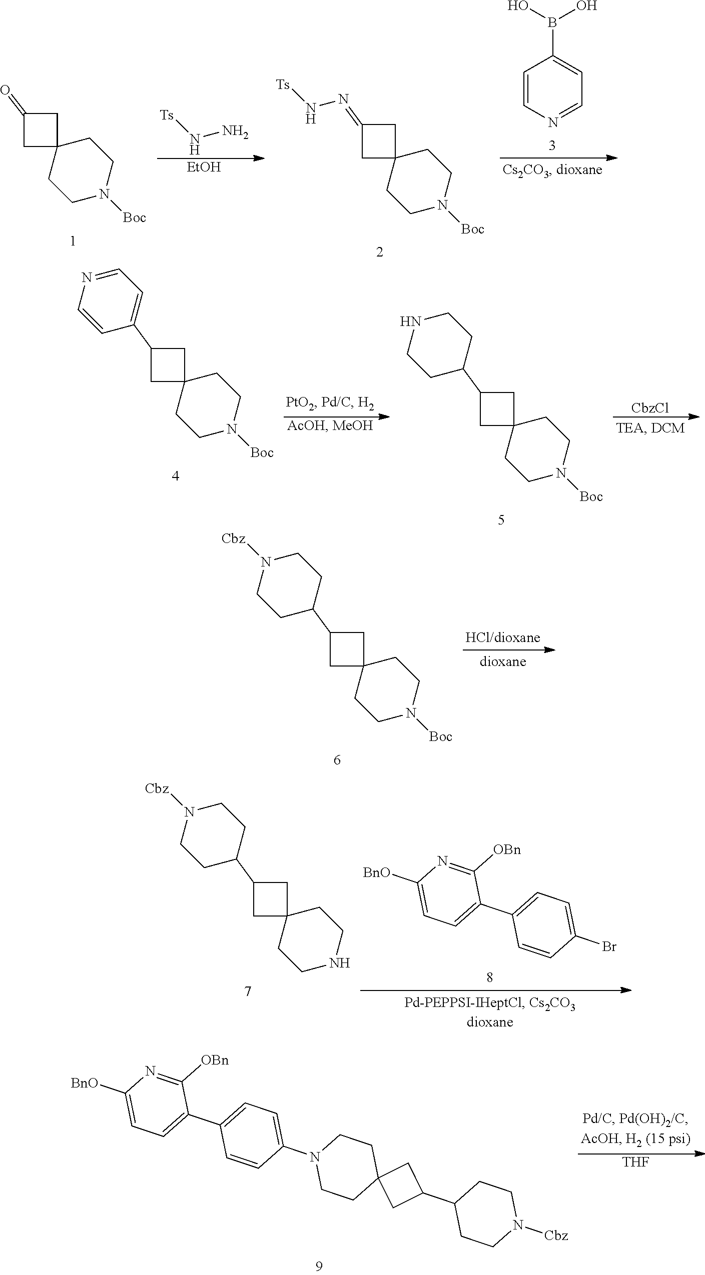

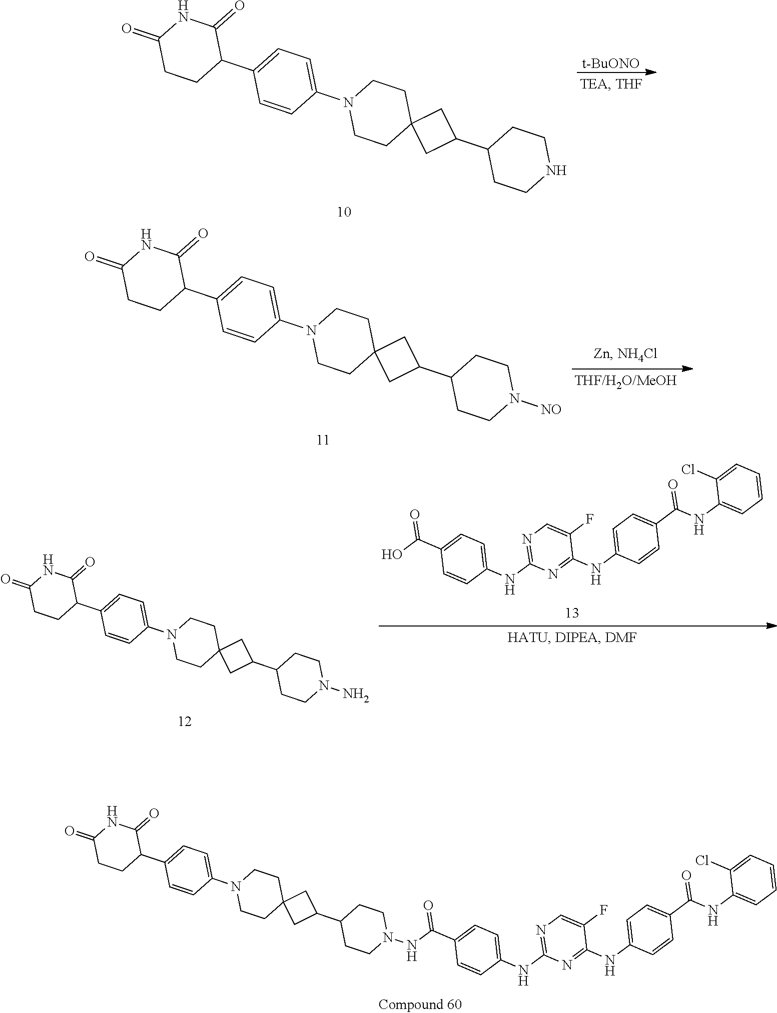

### Step 1. Synthesis of tert-butyl 2-(2-tosylhydrazineylidene)-7-azaspiro[3.5]nonane-7-carboxylate (2)

A mixture of tert-butyl 2-oxo-7-azaspiro[3.5]nonane-7-carboxylate (25 g, 104.47 mmol) and 4-methylbenzenesulfonohydrazide (19.46 g, 104.47 mmol) in EtOH (200 mL) was stirred at 80° C. for 2 hours. LCMS showed tert-butyl 2-oxo-7-azaspiro[3.5]nonane-7-carboxylate was consumed completely and 58% of desired mass was detected. The reaction mixture was cooled to 0° C., filtered and the filter cake was washed with 30 mL of cooled EtOH. The filter cake was collected and dried in vacuum to afford tert-butyl 2-(2-tosylhydrazineylidene)-7-azaspiro[3.5]nonane-7-carboxylate (16 g) as an off-white solid, which was used directly. MS(M+H)⁺=408.1.

¹H NMR (400 MHz, DMSO-d₆) δ=10.30 (s, 1H), 7.69 (d, J=8.2 Hz, 2H), 7.39 (d, J=8.2 Hz, 2H), 3.32-3.07 (m, 4H), 2.60-2.54 (m, 4H), 2.38 (s, 3H), 1.48-1.41 (m, 4H), 1.38 (s, 9H).

### Step 2. Synthesis of tert-butyl 2(pyridin-4-yl)-7-azaspiro[3.5]nonane-7-carboxylate (4)

A mixture of tert-butyl 2-(2-tosylhydrazineylidene)-7-azaspiro[3.5]nonane-7-carboxylate (14 g, 34.35 mmol), pyridin-4-ylboronic acid (8.45 g, 68.71 mmol) and Cs₂CO₃ (33.58 g, 103.06 mmol) in dioxane (200 mL) was stirred at 110° C. for 16 hours. LCMS showed tert-butyl 2-(2-tosylhydrazineylidene)-7-azaspiro[3.5]nonane-7-carboxylate was consumed completely and 5% of desired mass was detected. The mixture was combined with another batch (2 g), the resulting mixture was filtered and the filter cake was washed with EtOAc (500 mL). The filtrate was concentrated in vacuum. The residue was purified by flash silica gel chromatography (80 g SepaFlash® Silica Flash Column, eluent of 25-31% EtOAc in petroleum ether, gradient @ 200 mL/min) to afford tert-butyl 2-(pyridin-4-yl)-7-azaspiro[3.5]nonane-7-carboxylate (1.35 g, 2.86 mmol, 8.32% yield, 64% purity) as an off-white solid. MS(M+H)⁺=303.2.

### Step 3. Synthesis of tert-butyl 2-(piperidin-4-yl)-7-azaspiro[3.5]nonane-7-carboxylate (5)

To a solution of tert-butyl 2-(pyridin-4-yl)-7-azaspiro[3.5]nonane-7-carboxylate (1.6 g, 5.29 mmol) in MeOH (30 mL) were added PtO₂ (300 mg, 1.32 mmol), AcOH (317.72 mg, 5.29 mmol) and Pd/C (1 g, 10% purity) under N₂ atmosphere. The suspension was degassed under vacuum and purged with H₂ several times. The mixture was stirred under H₂ (50 Psi) atmosphere at 30° C. for 48 hours. LCMS showed the starting material was consumed completely and desired mass was detected. The mixture was filtered and the filter cake was washed with MeOH (100 mL). The filtrate was concentrated in vacuum. The residue was diluted with EtOAc and treated with Na₂CO₃ solution to adjust pH>7. The mixture was extracted with a mixture solvent of EtOAc/MeOH (10:1, 10 mL×10). The combined organic layers were washed brine, dried over Na₂SO₄, filtered and concentrated in vacuum to afford tert-butyl 2-(piperidin-4-yl)-7-azaspiro[3.5]nonane-7-carboxylate (1.62 g) as a gray gum, which was used directly. MS(M+H)⁺=309.3.

### Step 4. Synthesis of tert-butyl 2-(1-((benzyloxy)carbonyl)piperidin-4-yl)-7-azaspiro[3.5]nonane-7-carboxylate (6)

To a solution of tert-butyl 2-(piperidin-4-yl)-7-azaspiro[3.5]nonane-7-carboxylate (1.62 g, 5.25 mmol) and TEA (1.33 g, 13.13 mmol, 1.83 mL) in DCM (30 mL) was added CbzCl (1.16 g, 6.83 mmol, 974.67 μL) slowly at 0° C., the resulting mixture was stirred at 15° C. for 16 hours. LCMS showed the starting material was consumed completely. The mixture was concentrated in vacuum and the residue was purified by flash silica gel chromatography (40 g SepaFlash® Silica Flash Column, eluent of 3-12% EtOAc in petroleum ether, gradient @ 100 mL/min). The residue was purified by prep-TLC (SiO₂, petroleum ether:EtOAc=3:1) to afford tert-butyl 2-(1-((benzyloxy)carbonyl)piperidin-4-yl)-7-azaspiro[3.5]nonane-7-carboxylate (870 mg, 1.97 mmol, 37.43% yield) as a colorless oil. MS(M−Boc+H)⁺=343.2.

¹H NMR (400 MHz, DMSO-d₆) δ=7.41-7.28 (m, 5H), 5.05 (s, 2H), 4.03-3.93 (m, 2H), 3.28-3.22 (m, 2H), 3.19-3.12 (m, 2H), 2.87-2.68 (m, 2H), 1.96-1.88 (m, 1H), 1.87-1.79 (m, 2H), 1.64-1.56 (m, 2H), 1.50-1.44 (m, 2H), 1.42-1.39 (m, 1H), 1.39-1.28 (m, 13H), 0.92-0.76 (m, 2H).

### Step 5. Synthesis of benzyl 4-(7-azaspiro[3.5]nonan-2-yl)piperidine-1-carboxylate (7)

A mixture of tert-butyl 2-(1-((benzyloxy)carbonyl)piperidin-4-yl)-7-azaspiro[3.5]nonane-7-carboxylate (870 mg, 1.97 mmol) and HCl/dioxane (4 M, 15 mL) in dioxane (5 mL) was stirred at 15° C. for 2 hours. LCMS showed trace of the starting material remained and 89% peak with desired mass. The mixture was concentrated in vacuum to afford benzyl 4-(7-azaspiro[3.5]nonan-2-yl)piperidine-1-carboxylate (805 mg, HCl salt) as a white solid, which was used directly. MS(M+H)⁺=343.2.

### Step 6. Synthesis of benzyl 4-(7-(4-(2,6-bis(benzyloxyl)pyridin-3-yl)phenyl)-7-azaspiro[3.5]nonan-2-yl)piperidine-1-carboxylate (9)

A mixture of benzyl 4-(7-azaspiro[3.5]nonan-2-yl)piperidine-1-carboxylate (700 mg, 1.85 mmol, HCl salt), 2, 6-dibenzyloxy-3-(4-bromophenyl)pyridine (1 g, 2.24 mmol), Cs₂CO₃ (1.81 g, 5.54 mmol) and Pd-PEPPSI-IHeptCl (125.79 mg, 129.31 μmol) in dioxane (15 mL) was stirred at 100° C. for 16 hours under N₂ atmosphere. LCMS showed benzyl 4-(7-azaspiro[3.5]nonan-2-yl)piperidine-1-carboxylate was consumed completely and 63% of desired mass was detected. The mixture was diluted with H₂O (30 mL) and extracted with EtOAc (20 mL×3). The combined organic layers were dried over Na₂SO₄, filtered and concentrated in vacuum. The residue was purified by flash silica gel chromatography (20 g SepaFlash® Silica Flash Column, eluent of 10-40% EtOAc in petroleum ether, gradient @ 100 mL/min) to afford benzyl 4-(7-(4-(2,6-bis(benzyloxy)pyridin-3-yl)phenyl)-7-azaspiro[3.5]nonan-2-yl)piperidine-1-carboxylate (1.13 g, 1.60 mmol, 86.41% yield) as a yellow oil. MS(M+H)⁺=708.4.

### Step 7. Synthesis of 3-(4-(2-(piperidin-4-yl)-7-azaspiro[3.5]nonan-7-yl)phenyl)piperidine-2,6-dione (10)

To a mixture of Pd/C (1 g, 10% purity) and Pd(OH)₂/C (1 g, 20% purity) in THF (30 mL) under N₂ atmosphere were added benzyl 4-(7-(4-(2,6-bis(benzyloxy)pyridin-3-yl)phenyl)-7-azaspiro[3.5]nonan-2-yl)piperidine-1-carboxylate (1 g, 1.41 mmol) and AcOH (84.83 mg, 1.41 mmol). The resulting mixture was degassed under vacuum and purged with H₂ several times. The mixture was stirred under H₂ (15 psi) atmosphere at 30° C. for 40 hours. LCMS showed the starting material was consumed completely and desired mass was detected. The reaction mixture was filtered, washed with THF (100 mL) and MeOH (100 mL). The filtrate was concentrated in vacuum. The residue was triturated with a mixture solvents of MTBE:EtOAc (1:1, 20 mL) for 10 minutes. The mixture was filtered and washed with EtOAc (20 mL). The filter cake was collected and dried to afford 3-(4-(2-(piperidin-4-yl)-7-azaspiro[3.5]nonan-7-yl)phenyl)piperidine-2,6-dione (340 mg, 859.59 μmol, 60.85% yield) as a light yellow solid. MS(M+H)⁺=396.2.

### Step 8. Synthesis of 3-(4-(2-(1-nitrosopiperidin-4-yl)-7-azaspiro[3.5]nonan-7-yl)phenyl)piperidine-2,6-dione (11)

To a solution of 3-(4-(2-(piperidin-4-yl)-7-azaspiro[3.5]nonan-7-yl)phenyl)piperidine-2,6-dione (320 mg, 809.03 μmol) in THF (5 mL) was added TEA (245.59 mg, 2.43 mmol, 337.82 μL) at 0° C., followed by the addition of t-BuONO (834.27 mg, 8.09 mmol, 962.25 μL). The resulting mixture was stirred at 60° C. for 16 hours. LCMS showed 71% the starting material remained and 26% of desired mass was detected. To the mixture was added tert-butyl nitrite (433.50 mg, 4.20 mmol, 0.5 mL) and the resulting mixture was stirred at 60° C. for another 16 hours. LCMS showed the starting material was consumed completely. The mixture was concentrated in vacuum and purified by flash silica gel chromatography (20 g SepaFlash® Silica Flash Column, eluent of 50-100% EtOAc in petroleum ether, gradient @ 100 mL/min). The residue was triturated with MTBE (3 mL) and filtered. The filter cake was collected and dried to afford 3-(4-(2-(1-nitrosopiperidin-4-yl)-7-azaspiro[3.5]nonan-7-yl)phenyl)piperidine-2,6-dione (110 mg, 259.11 mol, 32.03% yield) as a brown solid. MS(M+H)⁺=425.2.

### Step 9. Synthesis of 3-(4-(2-(1-aminopiperidin-4-yl)-7-azaspiro[3.5]nonan-7-yl)phenyl)piperidine-2,6-dione (12)

To a solution of 3-(4-(2-(1-nitrosopiperidin-4-yl)-7-azaspiro[3.5]nonan-7-yl)phenyl)piperidine-2,6-dione (50 mg, 117.78 μmol) in THF (1 mL), H₂O (1 mL) and MeOH (1 mL) were added Zn (100 mg, 1.53 mmol) and NH₄Cl (25.20 mg, 471.10 μmol) at 0° C. The mixture was stirred at 20° C. for 4 hrs. LCMS showed the starting material was consumed completely and desired mass was detected. The mixture was filtered and the filter cake was washed with THF (10 mL). The filtrate was concentrated in vacuum to afford 3-(4-(2-(1-aminopiperidin-4-yl)-7-azaspiro[3.5]nonan-7-yl)phenyl)piperidine-2,6-dione (80 mg) as a light brown solid, which was used directly. MS(M+H)⁺=411.4.

### Step 10. Synthesis of N-(2-chlorophenyl)-4-((2-((4-((4-(7-(4-(2,6-dioxopiperidin-3-yl)phenyl)-7-azaspiro[3,5]nonan-2-yl)piperidin-1-yl)carbamoyl)phenyl)amino)-5-fluoropyrimidin-4-yl)amino)benzamide (Compound 60)

A mixture of 4-((4-((4-((2-chlorophenyl)carbamoyl)phenyl)amino)-5-fluoropyrimidin-2-yl)amino) benzoic acid (90 mg, 188.33 μmol), HATU (85.93 mg, 226.00 μmol) and DIPEA (121.70 mg, 941.67 μmol, 164.02 μL) in DMF (2 mL) was stirred at 20° C. for 15 minutes, then 3-(4-(2-(1-aminopiperidin-4-yl)-7-azaspiro[3.5]nonan-7-yl)phenyl)piperidine-2,6-dione (80 mg, 194.86 μmol) was added, the resulting mixture was stirred at 20° C. for 1 hour. LCMS showed 3-(4-(2-(1-aminopiperidin-4-yl)-7-azaspiro[3.5]nonan-7-yl)phenyl)piperidine-2,6-dione was consumed completely and desired mass was detected. The mixture was diluted with H₂O (10 mL) and extracted with a mixture solvents of EtOAc/THF (10/1, 10 mL×4). The combined organic layers were washed with brine (10 mL×3), dried over Na₂SO₄ and concentrated in vacuum. The residue was purified by prep-TLC (SiO₂, DCM:MeOH=10:1) twice and freeze-dried to afford N-(2-chlorophenyl)-4-((2-((4-((4-(7-(4-(2,6-dioxopiperidin-3-yl)phenyl)-7-azaspiro[3.5]nonan-2-yl)piperidin-1-yl)carbamoyl)phenyl)amino)-5-fluoropyrimidin-4-yl)amino)benzamide (2.9 mg, 2.85 mol, 35.38% yield, 85.4% purity) as an off-white solid. MS(M+H)⁺=870.3.

¹H NMR (400 MHz, DMSO-d₆) δ=10.77 (s, 1H), 9.97-9.89 (m, 1H), 9.74 (s, 1H), 9.64-9.56 (m, 1H), 9.18-9.08 (m, 1H), 8.25 (d, J=3.4 Hz, 1H), 8.08-7.97 (m, 4H), 7.82-7.73 (m, 2H), 7.73-7.62 (m, 3H), 7.56 (d, J=7.2 Hz, 1H), 7.43-7.36 (m, 1H), 7.34-7.25 (m, 1H), 7.02 (br d, J=8.7 Hz, 2H), 6.92-6.84 (m, 2H), 3.71 (br dd, J=5.0, 11.4 Hz, 1H), 3.12-3.05 (m, 2H), 3.03-2.95 (m, 4H), 2.65-2.60 (m, 2H), 2.06-1.93 (m, 4H), 1.92-1.84 (m, 2H), 1.70-1.59 (m, 4H), 1.57-1.52 (m, 2H), 1.48-1.39 (m, 3H), 1.16-1.09 (m, 3H).

### Example 61. Synthesis of 4-((4-((5-cyclopropyl-1H-pyrazol-3-yl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (Compound 61)

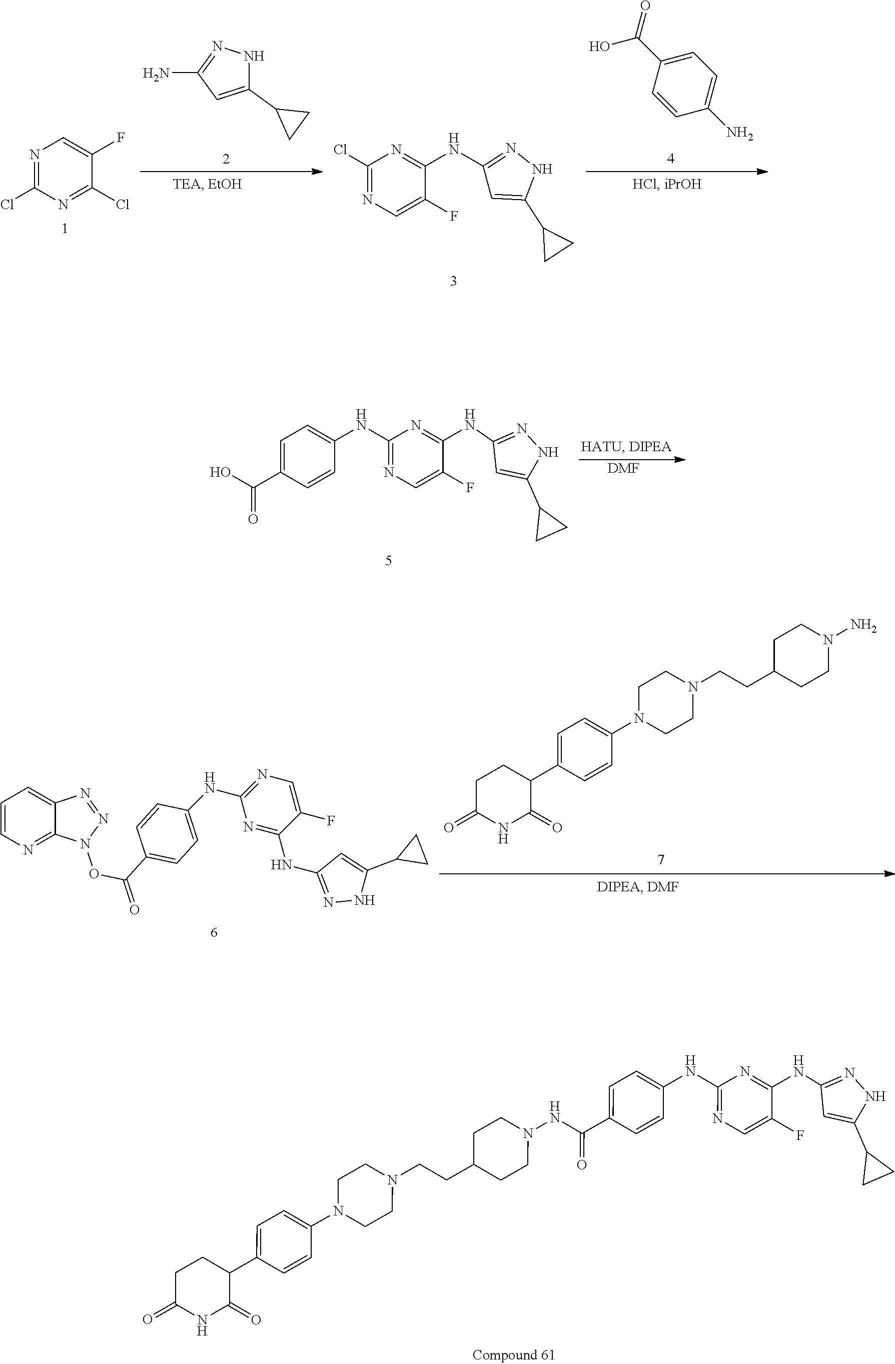

### Step 1. Synthesis of 2-chloro-N-(5-cyclopropyl-1H-pyrazol-3-yl)-5-fluoropyrimidin-4-amine (3)

To a solution of 2,4-dichloro-5-fluoropyrimidine (1 g, 5.99 mmol) and 5-cyclopropyl-1H-pyrazol-3-amine (737.60 mg, 5.99 mmol) in EtOH (10 mL) was added TEA (2.32 g, 17.97 mmol, 3.13 mL). The mixture was stirred at 80° C. for 16 hr. LCMS showed 2,4-dichloro-5-fluoropyrimidine was consumed completely and desired mass was detected. The reaction mixture was diluted with H₂O (30 mL) and extracted with EtOAc (10 mL×2). The combined organic layers were dried over Na₂SO₄, filtered and concentrated under reduced pressure. The residue was purified by flash silica gel chromatography (ISCO®; 20 g SepaFlash® Silica Flash Column, Eluent of 0~50% EtOAc/Petroleum ether, gradient @ 80 mL/min) to afford 2-chloro-N-(5-cyclopropyl-1H-pyrazol-3-yl)-5-fluoropyrimidin-4-amine (1 g, 3.27 mmol, 54.63% yield, 83% purity) as a white solid. MS(M+H)⁺=254.2.

### Step 2. Synthesis of 4-((4-((5-cyclopropyl-1H-pyrazol-3-yl)amino)-5-fluoropyrimidin-2-yl)amino)benzoic acid (5)

To a solution of 2-chloro-N-(5-cyclopropyl-1H-pyrazol-3-yl)-5-fluoropyrimidin-4-amine (0.5 g, 1.64 mmol) and 4-aminobenzoic acid (224.36 mg, 1.64 mmol) in iPrOH (5 mL) was added HCl (12 M, 409.01 μL). The mixture was stirred at 80° C. for 16 hr. LCMS showed 2-chloro-N-(5-cyclopropyl-1H-pyrazol-3-yl)-5-fluoropyrimidin-4-amine was consumed completely and desired mass was detected. The reaction mixture was diluted with H₂O (5 mL) and EtOAc (10 mL). The mixture was filtered. The filter cake was collected and dried to afford 4-((4-((5-cyclopropyl-1H-pyrazol-3-yl)amino)-5-fluoropyrimidin-2-yl)amino)benzoic acid (510 mg, 1.44 mmol, 87.98% yield) as a white solid. MS(M+H)⁺=355.3.

### Step 3. Synthesis of 3H-[1.2.3]triazolo[4,5-b]pyridin-3-yl 4-((4-((5-cyclopropyl-1H-pyrazol-3-yl)amino)-5-fluoropyrimidin-2-yl)amino)benzoate (6)

To a solution of 4-((4-((5-cyclopropyl-1H-pyrazol-3-yl)amino)-5-fluoropyrimidin-2-yl)amino) benzoic acid (100 mg, 282.22 μmol) in DMF (1 mL) were added HATU (160.96 mg, 423.32 mol) and DIPEA (109.42 mg, 846.65 μmol, 147.47 μL). After stirring at 25° C. for 0.5 hr, LCMS showed 4-((4-((5-cyclopropyl-1H-pyrazol-3-yl)amino)-5-fluoropyrimidin-2-yl)amino)benzoic acid was consumed completely and desired mass was detected. The reaction mixture was diluted with H₂O (20 mL) at 25° C., and then extracted with EtOAc (10 mL×2). The combined organic layers were washed with brine (20 mL), dried over Na₂SO₄, filtered and concentrated under reduced pressure. The residue was triturated with DCM/PE (5 mL, 10/1) at 25° C. for 1 hr to afford 3H-[1,2,3]triazolo[4,5-b]pyridin-3-yl 4-((4-((5-cyclopropyl-1H-pyrazol-3-yl)amino)-5-fluoropyrimidin-2-yl)amino)benzoate (50 mg, 105.83 μmol, 37.50% yield) as a white solid. MS(M+H)⁺=473.2.

### Step 4. Synthesis of 4-((4-((5-cyclopropyl-1H-pyrazol-3-yl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (Compound 61)

To a solution of 3H-[1,2,3]triazolo[4,5-b]pyridin-3-yl 4-((4-((5-cyclopropyl-1H-pyrazol-3-yl)amino)-5-fluoropyrimidin-2-yl)amino)benzoate (50 mg, 105.83 μmol) and 3-(4-(4-(2-(1-aminopiperidin-4-yl)ethyl)piperazin-1-yl)phenyl)piperidine-2,6-dione (48 mg, 93.47 μmol, TFA) in DMF (0.5 mL) was added DIPEA (41.04 mg, 317.50 mol, 55.30 μL). The mixture was stirred at 25° C. for 1 hr. LCMS showed 3-(4-(4-(2-(1-aminopiperidin-4-yl)ethyl)piperazin-1-yl)phenyl)piperidine-2,6-dione was consumed completely and desired mass was detected. The reaction mixture was diluted with H₂O (10 mL) and EtOAc (5 mL). The mixture was filtered. The filter cake was collected to give the crude product, which was purified by prep-HPLC (column: Phenomenex luna C18 150*25 mm*10 um; mobile phase: [water (TFA)-ACN]; gradient: 15%-45% B over 9 min) and lyophilized to afford 4-((4-((5-cyclopropyl-1H-pyrazol-3-yl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)benzamide (15.1 mg, 15.35 μmol, 14.51% yield, 98% purity, 2TFA) as a white solid. MS(M+H)⁺=736.3.

¹H NMR (400 MHz, DMSO-d₆) δ=10.79 (s, 1H), 10.00-9.89 (m, 1H), 9.68-9.43 (m, 3H), 8.11 (d, J=3.6 Hz, 1H), 7.82-7.65 (m, 4H), 7.12 (d, J=8.8 Hz, 2H), 6.98 (d, J=8.6 Hz, 2H), 6.19 (br s, 1H), 3.84-3.80 (m, 2H), 3.76-3.72 (m, 2H), 3.60 (br d, J=11.4 Hz, 2H), 3.26-3.19 (m, 2H), 3.17-3.10 (m, 3H), 3.05-2.92 (m, 2H), 2.90-2.76 (m, 2H), 2.67-2.60 (m, 1H), 2.47-2.45 (m, 1H), 2.22-2.10 (m, 1H), 2.05-1.95 (m, 1H), 1.94-1.85 (m, 1H), 1.80-1.70 (m, 2H), 1.65-1.55 (m, 2H), 1.47-1.32 (m, 3H), 0.99-0.87 (m, 2H), 0.76-0.63 (m, 2H).

### Example 62. Synthesis of N-(2-chlorophenyl)-4-((2-((4-(((1-((1-(4-(2,6-dioxopiperidin-3-yl)phenyl)piperidin-4-yl)methyl)piperidin-4-yl)methyl)carbamoyl)phenyl)amino)-5-fluoropyrimidin-4-yl)amino)benzamide (Compound 62)

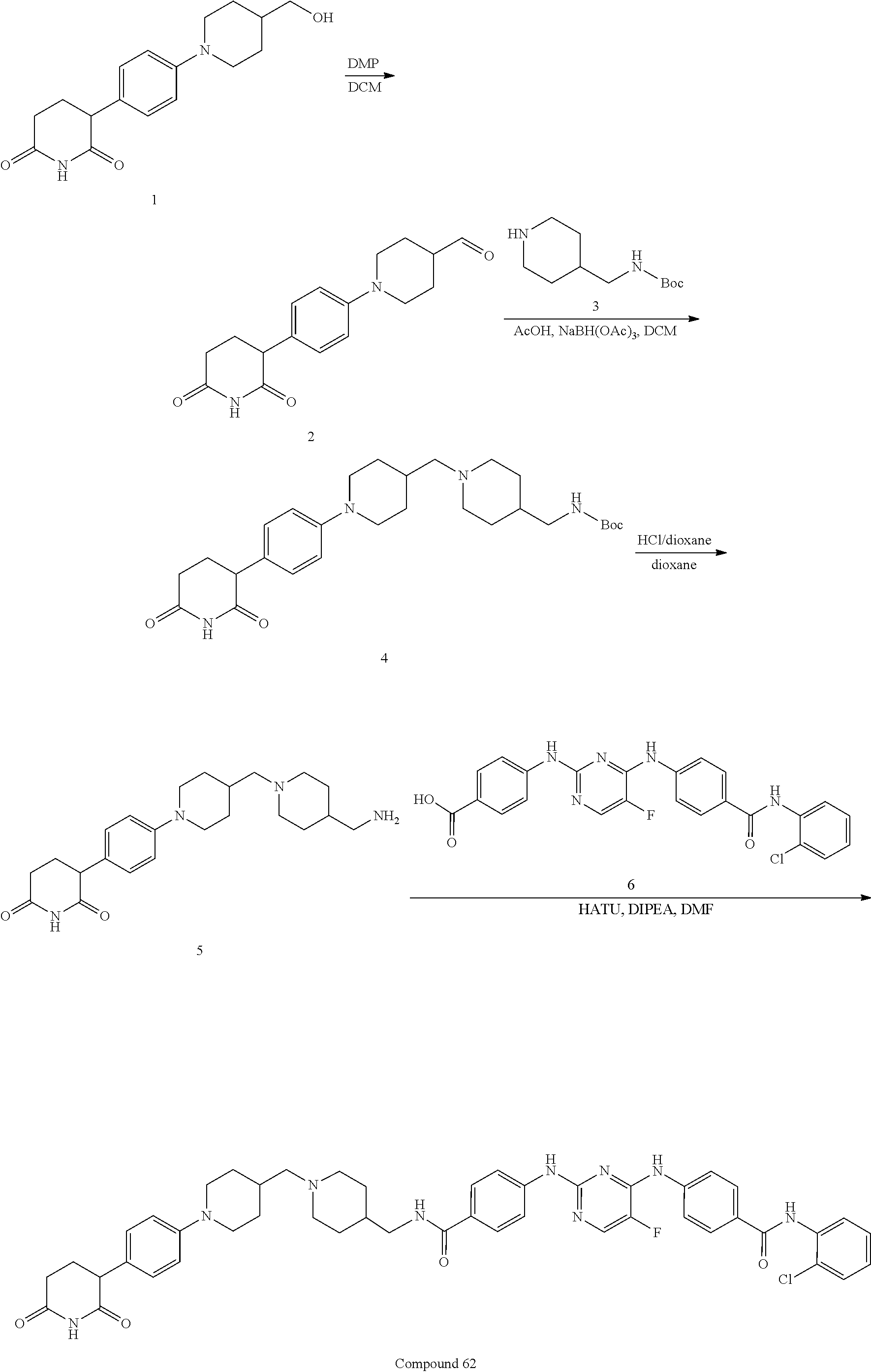

### Step 1. Synthesis of 1-(4-(2,6-dioxopiperidin-3-yl)phenyl)piperidine-4-carbaldehyde (2)

To a solution of 3-(4-(4-(hydroxymethyl)piperidin-1-yl)phenyl)piperidine-2,6-dione (1 g, 3.31 mmol) in DCM (5 mL) was added DMP (1.54 g, 3.64 mmol, 1.13 mL). The mixture was stirred at 25° C. for 1 hr. LCMS showed 3-(4-(4-(hydroxymethyl)piperidin-1-yl)phenyl)piperidine-2,6-dione was consumed completely and desired mass was detected. The reaction mixture was diluted with 10% Na₂SO₃ solution (100 mL) and extracted with EtOAc (20 mL×2). The combined organic layers were dried over Na₂SO₄, filtered and concentrated under reduced pressure to afford 1-(4-(2,6-dioxopiperidin-3-yl)phenyl)piperidine-4-carbaldehyde (1 g, crude) a brown solid. MS(M+H)⁺=301.2.

### Step 2. Synthesis of tert-butyl ((1-((1-(4-(2,6-dioxopiperidin-3-yl)phenyl)piperidin-4-yl)methyl)piperidin-4-yl)methyl)carbamate (4)

To a solution of 1-(4-(2,6-dioxopiperidin-3-yl)phenyl)piperidine-4-carbaldehyde (300 mg, 998.83 μmol) and tert-butyl (piperidin-4-ylmethyl)carbamate (192.65 mg, 898.95 μmol) in DCM (1 mL) was added AcOH (59.98 mg, 998.83 μmol, 57.18 μL). After stirring at 25° C. for 0.5 hr, NaBH(OAc)₃ (635.08 mg, 3.00 mmol) was added and the resulting mixture was stirred at 25° C. for 16 hr. LCMS showed 1-(4-(2,6-dioxopiperidin-3-yl)phenyl)piperidine-4-carbaldehyde was consumed completely and desired mass was detected. The reaction mixture was diluted with H₂O (50 mL) and extracted with EtOAc (20 mL×3). The water layer was added saturated NaHCO₃ solution to adjust pH=8.0 and extracted with EtOAc (30 mL×2). The combined organic layers dried over Na₂SO₄, filtered and concentrated under reduced pressure to afford tert-butyl ((1-((1-(4-(2,6-dioxopiperidin-3-yl)phenyl)piperidin-4-yl)methyl)piperidin-4-yl)methyl)carbamate (170 mg, 340.92 μmol, 34.13% yield) as a brown solid. MS(M+H)⁺=499.3.

### Step 3. Synthesis of 3-(4-(4-((4-(aminomethyl)piperidin-1-yl)methyl)piperidin-1-yl)phenylpiperidine-2,6-dione (5)

To a solution of tert-butyl ((1-((1-(4-(2,6-dioxopiperidin-3-yl)phenyl)piperidin-4-yl)methyl)piperidin-4-yl)methyl)carbamate (100 mg, 200.54 μmol) in dioxane (2 mL) was added HCl/dioxane (4 M, 4 mL). The mixture was stirred at 25° C. for 50 min. LCMS showed the starting material was consumed completely and desired mass was detected. The reaction mixture was concentrated under reduced pressure to afford 3-(4-(4-((4-(aminomethyl)piperidin-1-yl)methyl)piperidin-1-yl)phenyl)piperidine-2,6-dione (87 mg, crude, HCl) as a white solid. MS(M+H)⁺=399.3.

### Step 4. Synthesis of N-(2-chlorophenyl)-4-((2-((4-(((1-((1-(4-(2,6-dioxopiperidin-3-yl)phenyl)piperidin-4-yl) methyl)piperidin-4-yl)methyl)carbamoyl)phenyl)amino)-5-fluoropyrimidin-4-yl)amino)benzamide (Compound 62)

To a solution of 4-((4-((4-((2-chlorophenyl)carbamoyl)phenyl)amino)-5-fluoropyrimidin-2-yl)amino)benzoic acid (92.28 mg, 193.10 μmol) in DMF (1 mL) were added HATU (110.14 mg, 289.65 μmol) and DIPEA (124.79 mg, 965.51 μmol, 168.18 μL). After stirring at 25° C. for 0.5 hr, 3-(4-(4-((4-(aminomethyl)piperidin-1-yl)methyl)piperidin-1-yl)phenyl)piperidine-2,6-dione (84 mg, 193.10 μmol, HCl) was added and the mixture was stirred at 25° C. for 1 hr. LCMS showed 4-((4-((4-((2-chlorophenyl)carbamoyl)phenyl)amino)-5-fluoropyrimidin-2-yl)amino)benzoic acid was consumed completely and desired mass was detected. The reaction mixture was diluted with H₂O (20 mL) at 25° C., and then extracted with EtOAc (10 mL×3). The combined organic layers were washed with brine (10 mL), dried over Na₂SO₄, filtered and concentrated under reduced pressure. The residue was purified by prep-HPLC (column: Waters Xbridge 150*25 mm 10 um; mobile phase: [water (NH₄ HCO₃)-ACN]; gradient: 38%-68% B over 10 min) and lyophilized to afford N-(2-chlorophenyl)-4-((2-((4-(((1-((1-(4-(2,6-dioxopiperidin-3-yl)phenyl)piperidin-4-yl) methyl)piperidin-4-yl)methyl)carbamoyl)phenyl)amino)-5-fluoropyrimidin-4-yl)amino) benzamide (76.5 mg, 81.99 mol, 42.46% yield, 92% purity) as a white solid. MS(M+H)⁺=858.3.

¹H NMR (400 MHz, DMSO-d₆) δ=10.76 (s, 1H), 9.92 (s, 1H), 9.73 (s, 1H), 9.60 (s, 1H), 8.25 (d, J=3.4 Hz, 2H), 8.07-7.97 (m, 4H), 7.80-7.76 (m, 4H), 7.68-7.63 (m, 1H), 7.59-7.53 (m, 1H), 7.43-7.36 (m, 1H), 7.32-7.25 (m, 1H), 7.02 (d, J=8.7 Hz, 2H), 6.87 (d, J=8.8 Hz, 2H), 3.75-3.68 (m, 1H), 3.65-3.55 (m, 2H), 3.15-3.10 (m, 2H), 2.90-2.80 (m, 2H), 2.65-2.58 (m, 3H), 2.47-2.42 (m, 1H), 2.16-2.07 (m, 3H), 2.05-1.96 (m, 1H), 1.86-1.72 (m, 4H), 1.69-1.58 (m, 3H), 1.57-1.46 (m, 1H), 1.22-1.11 (m, 4H).

### Example 63. Synthesis of N-(2-chlorophenyl)-4-((2-((4-((4-((2S)-1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (Compound 63)

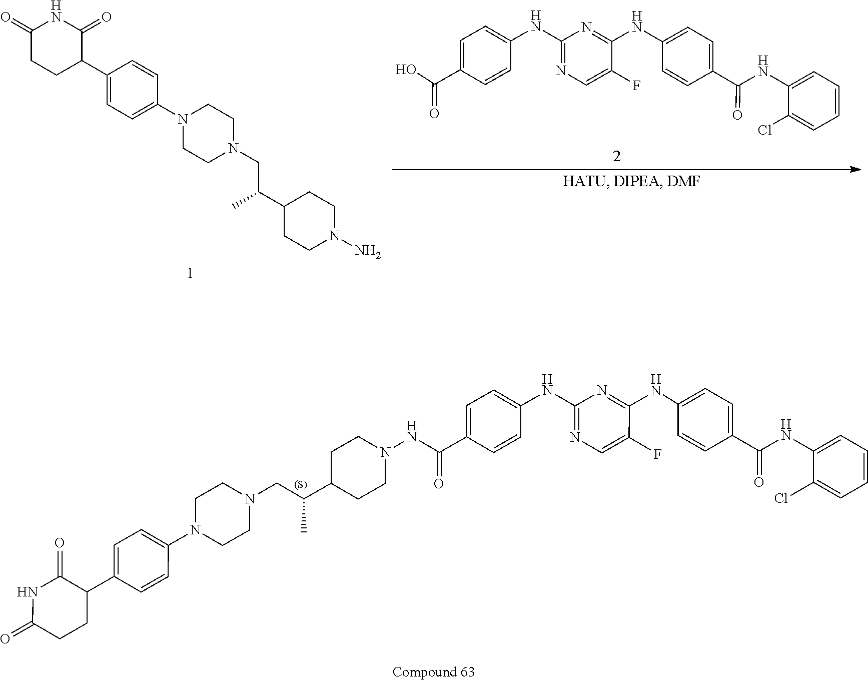

### Step 1. Synthesis of N-(2-chlorophenyl)-4-((2-((4-((4-((2S)-1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (Compound 63)

To a solution of 4-((4-((4-((2-chlorophenyl)carbamoyl)phenyl)amino)-5-fluoropyrimidin-2-yl)amino)benzoic acid (0.2 g, 418.52 μmol) and HATU (198.59 mg, 522.30 μmol) in DMF (1.5 mL) was added DIPEA (281.96 mg, 2.18 mmol, 380 μL) and the mixture was stirred at 20° C. for 15 min. Then 3-(4-(4-((S)-2-(1-aminopiperidin-4-yl)propyl)piperazin-1-yl)phenyl)piperidine-2,6-dione (180 mg, 435.25 μmol) in DMF (1.5 mL) was added and the mixture was stirred at 20° C. for 2 h. LCMS showed 38% peak of the desired mass. The mixture was diluted with water (20 mL) and then filtered. The filter cake was washed with water (10 mL), collected and dried to afford the crude product, which was purified by prep-TLC (DCM/MeOH=10/1) and then re-purified by prep-TLC (DCM/MeOH=9/1). The product was further purified by prep-HPLC (column: Phenomenex Luna C18 150*25 mm*Oum; mobile phase: [water (FA)-ACN]; B %: %, isocratic elution mode) and lyophilized to afford N-(2-chlorophenyl)-4-((2-((4-((4-((2S)-1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (7.7 mg, 8.55 mol, 1.96% yield, 97% purity) as a white solid. MS(M+H)⁺=873.4.

SFC Method Details: Column: Chiralpak IA-3 50×4.6 mm I. D., 3 um Mobile phase: A1=ACN (0.05% DEA); B1=EtOH (0.05% DEA) Gradient elution: EtOH (0.05% DEA) 95; ACN (0.05% DEA) 5% Flow rate: 1 mL/min Column Temp: 35° C.

¹H NMR (400 MHz, DMSO-d₆) δ=10.16-9.72 (m, 1H), 9.58-9.43 (m, 1H), 9.22-9.05 (m, 1H), 8.25-8.10 (m, 1H), 8.02-7.93 (m, 4H), 7.79-7.74 (m, 2H), 7.71-7.63 (m, 3H), 7.57-7.52 (m, 1H), 7.42-7.35 (m, 1H), 7.30-7.23 (m, 1H), 7.08-7.01 (m, 2H), 6.92-6.86 (m, 2H), 3.76-3.68 (m, 1H), 3.29-3.23 (m, 4H), 3.16-3.06 (m, 4H), 3.04-2.97 (m, 2H), 2.74-2.58 (m, 3H), 2.43-2.39 (m, 1H), 2.24-2.23 (m, 1H), 2.16-2.06 (m, 2H), 2.05-1.95 (m, 1H), 1.72-1.63 (m, 1H), 1.60-1.50 (m, 2H), 1.49-1.37 (m, 1H), 1.33-1.22 (m, 2H), 0.89-0.82 (m, 3H).

### Example 64. Synthesis of N-(2-chlorophenyl)-4-((2-((4-(((3R)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl) amino)benzamide (Compound 64)

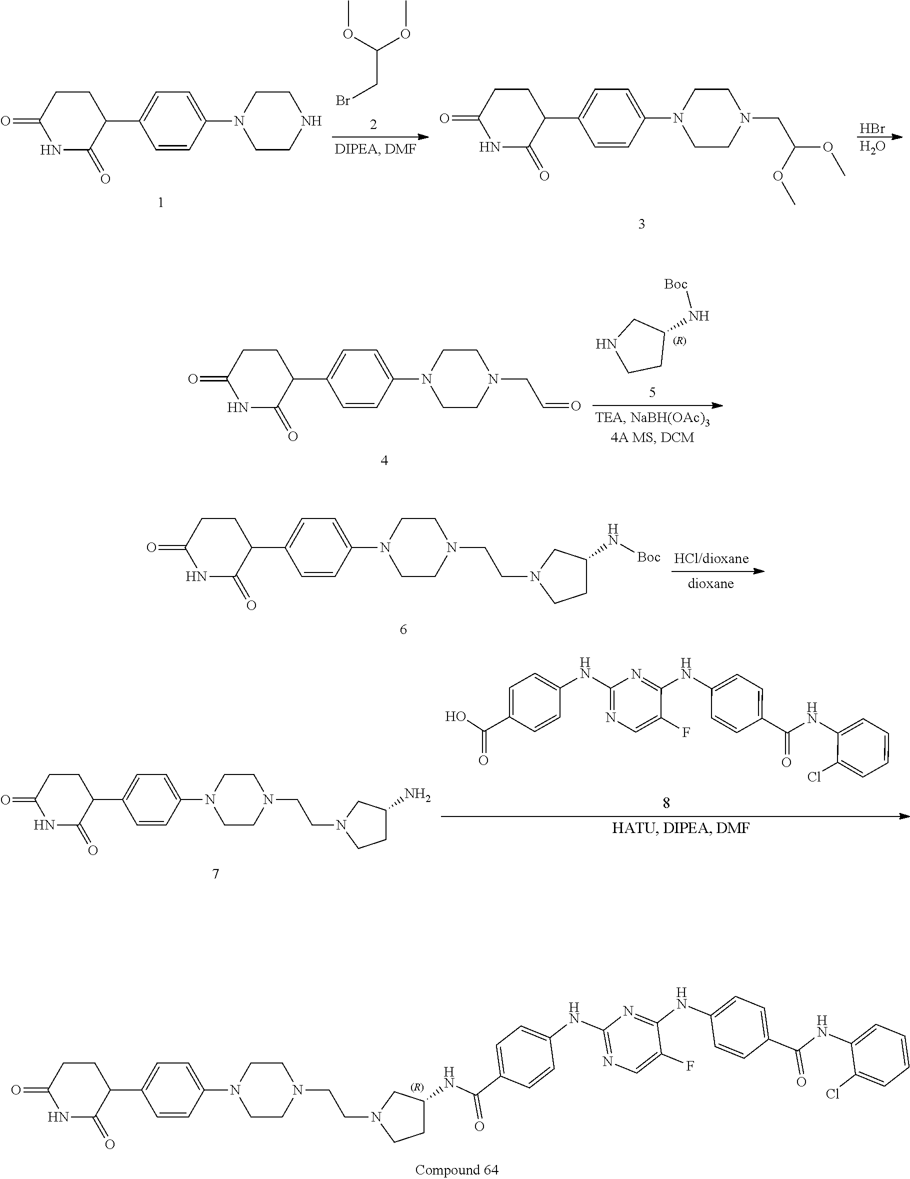

Step 1. Synthesis of 3-(4-(4-(2,2-dimethoxyethyl)piperazin-1-yl)phenyl)piperidine-2,6-dione (3)

To a solution of 3-(4-(piperazin-1-yl)phenyl)piperidine-2,6-dione (2 g, 6.46 mmol, HCl salt) and 2-bromo-1,1-dimethoxyethane (1.42 g, 8.39 mmol, 985.08 μL) in DMF (20 mL) was added DIPEA (4.17 g, 32.28 mmol, 5.62 mL). The mixture was stirred at 100° C. for 16 hr. LCMS showed 3-(4-(piperazin-1-yl)phenyl)piperidine-2,6-dione was consumed completely and one main peak with desired mass was detected. The mixture was diluted with H₂O (30 mL) and treated with saturated Na₂CO₃ solution (30 mL), the resulting mixture was extracted with EtOAc (20 mL×7). The combined organic layers were washed with brine (20 mL×3), dried over Na₂SO₄, filtered and concentrated in vacuum. The residue was purified by flash silica gel chromatography (ISCO®; 40 g SepaFlash® Silica Flash Column, Eluent of 90-100% EtOAc/Petroleum ether gradient @ 80 mL/min) to afford 3-(4-(4-(2,2-dimethoxyethyl)piperazin-1-yl)phenyl)piperidine-2,6-dione (740 mg, 2.05 mmol, 31.71% yield) as a light yellow solid. MS(M+H)⁺=362.2.

### Step 2. Synthesis of 2-(4-(4-(2,6-dioxopiperidin-3-yl)phenyl)piperazin-1-yl)acetaldehyde (4)

To a solution of 3-(4-(4-(2,2-dimethoxyethyl)piperazin-1-yl)phenyl)piperidine-2,6-dione (600 mg, 1.66 mmol) in H₂O (3 mL) was added HBr (8.94 g, 44.20 mmol, 6.00 mL, 40% purity). The mixture was stirred at 40° C. for 16 hr. LCMS showed 3-(4-(4-(2,2-dimethoxyethyl)piperazin-1-yl)phenyl)piperidine-2,6-dione was consumed completely. The reaction mixture was adjusted pH=7 by NaHCO₃ aq at 0° C., and extracted with EtOAc (20 mL×5). The combined organic layers were dried over Na₂SO₄, filtered and concentrated under reduced pressure to afford 2-(4-(4-(2,6-dioxopiperidin-3-yl)phenyl)piperazin-1-yl)acetaldehyde (300 mg, 951.28 mol, 57.30% yield) as a light yellow solid. MS(M+H)⁺=316.2, MS(M+H₂O+H)+=334.2.

### Step 3. Synthesis of tert-butyl ((3R)-1-(2-(4-(4-(2,6-dioxopiperidin-3-yl)phenyl)piperazin-1-yl)ethyl)pyrrolidin-3-yl) carbamate (6)

To a solution of 2-(4-(4-(2,6-dioxopiperidin-3-yl)phenyl)piperazin-1-yl)acetaldehyde (170 mg, 539.06 μmol) and tert-butyl (R)-pyrrolidin-3-ylcarbamate (130.52 mg, 700.77 μmol) in DCM (5 mL) was added TEA (54.55 mg, 539.06 μmol, 75.03 μL) and 4 Å MS (30 mg). After half an hour, NaBH(OAc)₃ (342.74 mg, 1.62 mmol) was added and the mixture was stirred at 20° C. for 16 hr. LCMS showed 2-(4-(4-(2,6-dioxopiperidin-3-yl)phenyl)piperazin-1-yl)acetaldehyde was consumed completely and one main peak with desired mass was detected. The residue was diluted with NaHCO₃ solution (20 mL) and extracted with DCM (20 mL×3). The combined organic layers were washed with water (20 mL), dried over Na₂SO₄, filtered and concentrated under reduced pressure. The residue was purified by flash silica gel chromatography (ISCO®; 20 g SepaFlash® Silica Flash Column, Eluent of 30-40% MeOH/EtOAc gradient @ 80 mL/min) to afford tert-butyl ((3R)-1-(2-(4-(4-(2,6-dioxopiperidin-3-yl)phenyl)piperazin-1-yl)ethyl)pyrrolidin-3-yl) carbamate (150 mg, 308.89 mol, 57.30% yield) as a white solid. MS(M+H)⁺=486.2.

### Step 4. Synthesis of 3-(4-(4-(2-((R)-3-aminopyrrolidin-1-yl)ethyl)piperazin-1-yl)phenyl)piperidine-2,6-dione (7)

To a solution of tert-butyl ((3R)-1-(2-(4-(4-(2,6-dioxopiperidin-3-yl)phenyl)piperazin-1-yl)ethyl)pyrrolidin-3-yl) carbamate (140 mg, 288.29 μmol) in dioxane (2 mL) was added HCl/dioxane (4 M, 2 mL). The mixture was stirred at 20° C. for 1 hr. LCMS showed the starting material was consumed completely and one main peak with desired mass was detected. The reaction mixture was concentrated under reduced pressure to remove solvent to afford 3-(4-(4-(2-((R)-3-aminopyrrolidin-1-yl)ethyl)piperazin-1-yl)phenyl)piperidine-2,6-dione (135 mg, 287.94 μmol, 99.88% yield, 90% purity, HCl salt) as a white solid. MS(M+H)⁺=386.2.

### Step 5. Synthesis of N-(2-chlorophenyl)-4-((2-((4-(((3R)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl) amino)benzamide (Compound 64)

To a solution of 4-((4-((4-((2-chlorophenyl)carbamoyl)phenyl)amino)-5-fluoropyrimidin-2-yl)amino)benzoic acid (114 mg, 238.56 μmol) and 3-(4-(4-(2-((R)-3-aminopyrrolidin-1-yl)ethyl)piperazin-1-yl)phenyl)piperidine-2,6-dione (134.22 mg, 286.27 mol, HCl salt) in DMF (2 mL) was added HATU (136.06 mg, 357.83 μmol) and DIPEA (154.16 mg, 1.19 mmol, 207.76 μL). The mixture was stirred at 20° C. for 2 hr. LCMS showed one main peak with desired mass was detected. The reaction mixture was diluted with water (30 mL) and extracted with THF (30 mL×3). The combined organic layers were washed with brine (30 mL×3), dried over Na₂SO₄, filtered and concentrated under reduced pressure to give a residue. The crude product was triturated with DCM (20 mL) at 25° C. for 30 min. The residue was purified by prep-HPLC (column: Waters Xbridge C18 150×50 mm×10 μm; mobile phase: [water(NH₄HCO₃)-ACN]; gradient: 28%-58% B over 10 min) and lyophilized to afford N-(2-chlorophenyl)-4-((2-((4-(((3R)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl)amino)benzamide (24.8 mg, 26.99 mol, 11.31% yield, 93% purity) as a white solid. MS(M+H)⁺=845.3.

¹H NMR (400 MHz, DMSO-d₆) δ=10.76 (s, 1H), 9.92 (s, 1H), 9.74 (s, 1H), 9.61 (s, 1H), 8.25 (d, J=3.5 Hz, 1H), 8.20 (br d, J=7.0 Hz, 1H), 8.05-7.99 (m, 4H), 7.81-7.75 (m, 4H), 7.66 (dd, J=1.5, 7.9 Hz, 1H), 7.56 (dd, J=1.3, 7.9 Hz, 1H), 7.42-7.36 (m, 1H), 7.32-7.26 (m, 1H), 7.03 (d, J=8.7 Hz, 2H), 6.87 (d, J=8.7 Hz, 2H), 4.40-4.29 (m, 1H), 3.71 (dd, J=4.8, 11.1 Hz, 1H), 3.14-3.05 (m, 4H), 2.85-2.75 (m, 1H), 2.66-2.60 (m, 2H), 2.60-2.56 (m, 2H), 2.56-2.53 (m, 4H), 2.48-2.41 (m, 5H), 2.15-2.05 (m, 2H), 2.03-1.96 (m, 1H), 1.79-1.70 (m, 1H).

### Example 65. Synthesis of N-(2-chlorophenyl)-4-((2-((4-((1-(2-(4-(4-(2,6-dioxopiperidin-3-yl)phenyl)piperazin-1-yl)ethyl)azetidin-3-yl)carbamoyl)phenyl)amino)-5-fluoropyrimidin-4-yl)amino) benzamide (Compound 65)

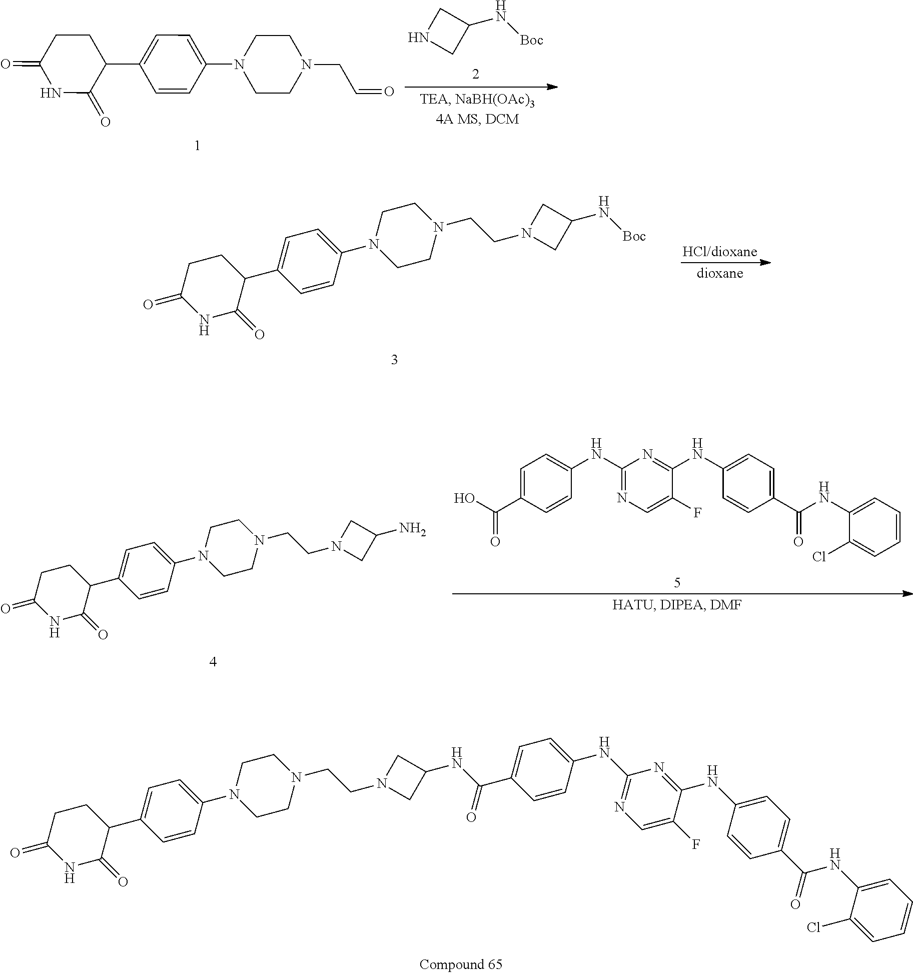 [SMILES: O=CCN1CCN(c2ccc(C3CCC(=O)NC3=O)cc2)CC1 ; CC(C)(C)OC(=O)NC1CNC1 ; CC(C)(C)OC(=O)NC1CN(CCN2CCN(c3ccc(C4CCC(=O)NC4=O)cc3)CC2)C1 ; NC1CN(CCN2CCN(c3ccc(C4CCC(=O)NC4=O)cc3)CC2)C1 ; O=C(O)c1ccc(Nc2ncc(F)c(Nc3ccc(C(=O)Nc4ccccc4Cl)cc3)n2)cc1 ; O=C1CCC(c2ccc(N3CCN(CCN4CC(NC(=O)c5ccc(Nc6ncc(F)c(Nc7ccc(C(=O)Nc8ccccc8Cl)cc7)n6)cc5)C4)CC3)cc2)C(=O)N1]

### Step 1. Synthesis of tert-butyl (1-(2-(4-(4-(2,6-dioxopiperidin-3-yl)phenyl)piperazin-1-yl)ethyl) azetidin-3-yl)carbamate (3)

To a solution of 2-(4-(4-(2,6-dioxopiperidin-3-yl)phenyl)piperazin-1-yl)acetaldehyde (170 mg, 539.06 μmol) and tert-butyl azetidin-3-ylcarbamate (120.69 mg, 700.77 mol) in DCM (5 mL) was added TEA (54.55 mg, 539.06 mol, 75.03 μL) and 4 Å MS (30 mg). After stirring for 30 mins, NaBH(OAc)₃ (342.74 mg, 1.62 mmol) was added and the resulting mixture was stirred at 20° C. for 16 hr. LCMS showed one main peak with desired mass was detected. The residue was diluted with NaHCO₃ solution (30 mL) and extracted with DCM (10 mL×3). The combined organic layers were washed with water (10 mL), dried over Na₂SO₄, filtered and concentrated under reduced pressure. The residue was purified by flash silica gel chromatography (ISCO®; 20 g SepaFlash® Silica Flash Column, Eluent of 30~40% MeOH/EtOAc gradient @ 80 mL/min) to afford tert-butyl (1-(2-(4-(4-(2,6-dioxopiperidin-3-yl)phenyl)piperazin-1-yl)ethyl)azetidin-3-yl)carbamate (200 mg, 424.10 μmol, 78.67% yield) as a white solid. MS(M+H)⁺=472.3.

### Step 2. Synthesis of 3-(4-(4-(2-(3-aminoazetidin-1-yl)ethyl)piperazin-1-yl)phenyl)piperidine-2,6-dione (4)

To a solution of tert-butyl (1-(2-(4-(4-(2,6-dioxopiperidin-3-yl)phenyl)piperazin-1-yl)ethyl) azetidin-3-yl)carbamate (190 mg, 402.89 μmol) in dioxane (2 mL) was added HCl/dioxane (4 M, 2 mL). The mixture was stirred at 20° C. for 1 hr. LCMS showed the starting material was consumed completely and one main peak with desired mass was detected. The reaction mixture was concentrated under reduced pressure to remove solvent to afford 3-(4-(4-(2-(3-aminoazetidin-1-yl)ethyl)piperazin-1-yl)phenyl)piperidine-2,6-dione (175 mg, 398.96 mol, 99.02% yield, 93% purity, HCl salt) as a white solid. MS(M+H)⁺=372.2.

### Step 3. Synthesis of N-(2-chlorophenyl)-4-((2-((4-((1-(2-(4-(4-(2,6-dioxopiperidin-3-yl)phenyl)piperazin-1-yl)ethyl)azetidin-3-yl)carbamoyl)phenyl)amino)-5-fluoropyrimidin-4-yl)amino) benzamide (Compound 65)

To a solution of 4-((4-((4-((2-chlorophenyl)carbamoyl)phenyl)amino)-5-fluoropyrimidin-2-yl)amino)benzoic acid (155 mg, 324.35 μmol) and 3-(4-(4-(2-(3-aminoazetidin-1-yl)ethyl)piperazin-1-yl)phenyl)piperidine-2,6-dione (174.48 mg, 389.22 μmol, 93%, HCl salt) in DMF (4 mL) was added HATU (184.99 mg, 486.53 μmol) and DIPEA (209.60 mg, 1.62 mmol, 282.48 μL). The mixture was stirred at 20° C. for 2 hr. LCMS showed one main peak with desired mass was detected. The reaction mixture was diluted with water (30 mL) and extracted with THF (20 mL×3). The combined organic layers were washed with brine (10 mL×3), dried over Na₂SO₄, filtered and concentrated under reduced pressure. The crude product was triturated with DCM (20 mL) at 25° C. for 30 min. The residue was purified by prep-HPLC (column: Waters Xbridge C18 150×50 mm×10 μm; mobile phase: [water(NH₄HCO₃)-ACN]; gradient: 28%-58% B over 10 min) and lyophilized to afford N-(2-chlorophenyl)-4-((2-((4-((1-(2-(4-(4-(2,6-dioxopiperidin-3-yl)phenyl)piperazin-1-yl)ethyl)azetidin-3-yl)carbamoyl)phenyl)amino)-5-fluoropyrimidin-4-yl) amino)benzamide (41.9 mg, 44.86 μmol, 13.83% yield, 89% purity) as a white solid. MS(M+H)⁺=831.3.

¹H NMR (400 MHz, DMSO-d₆) δ=10.77 (s, 1H), 9.92 (s, 1H), 9.74 (s, 1H), 9.63 (s, 1H), 8.52 (br d, J=7.0 Hz, 1H), 8.25 (d, J=3.5 Hz, 1H), 8.05-7.99 (m, 4H), 7.80-7.76 (m, 4H), 7.66 (dd, J=1.4, 8.0 Hz, 1H), 7.56 (dd, J=1.3, 7.9 Hz, 1H), 7.42-7.37 (m, 1H), 7.32-7.26 (m, 1H), 7.04 (d, J=8.7 Hz, 2H), 6.88 (d, J=8.8 Hz, 2H), 4.49-4.41 (m, 1H), 3.72 (dd, J=4.8, 10.9 Hz, 1H), 3.57 (br t, J=6.9 Hz, 2H), 3.13-3.05 (m, 4H), 2.97 (br t, J=7.1 Hz, 2H), 2.69-2.63 (m, 1H), 2.63-2.56 (m, 2H), 2.56-2.53 (m, 4H), 2.47-2.41 (m, 1H), 2.33-2.28 (m, 2H), 2.18-2.07 (m, 1H), 2.04-1.96 (m, 1H).

### Example 66. Synthesis of N-(2-chlorophenyl)-4-[[2-[4-[[4-[[1-[4-[(2,6-dioxo-3-piperidyl)amino]-2-fluoro-phenyl]-4-piperidyl]methyl-methyl-amino]cyclohexyl]carbamoyl]anilino]-5-fluoro-pyrimidin-4-yl]amino]benzamide (Compound 66)

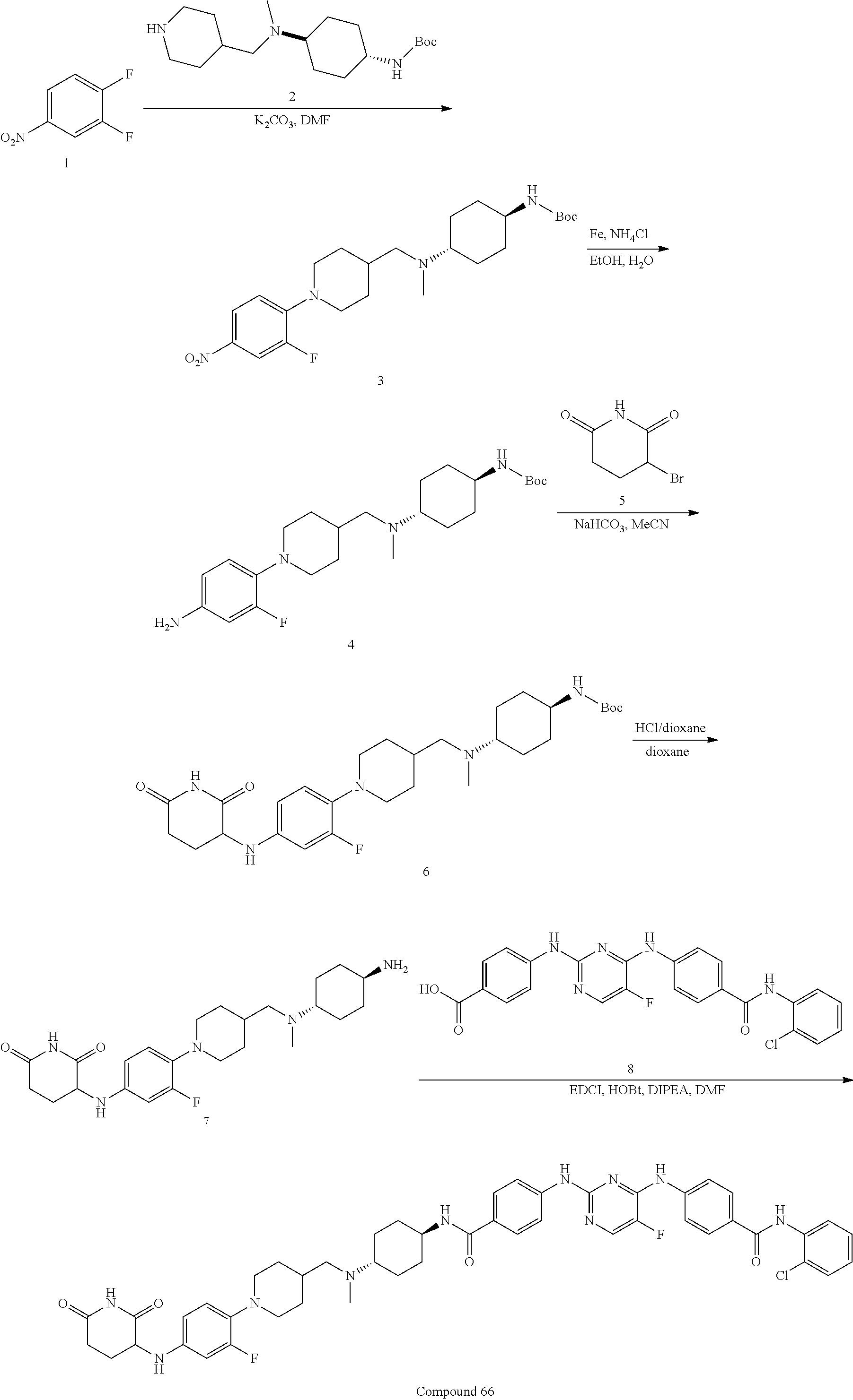

### Step 1. Synthesis of tert-butyl N-[4-[[1-(2-fluoro-4-nitro-phenyl)-4-piperidyl]methyl-methyl-amino]cyclohexyl]carbamate (3)

To a solution of 1, 2-difluoro-4-nitro-benzene (300 mg, 1.89 mmol) and tert-butyl N-[4-[methyl(4-piperidylmethyl) amino]cyclohexyl]carbamate (613.78 mg, 1.89 mmol) in DMF (6 mL) was added K₂CO₃ (781.85 mg, 5.66 mmol). The mixture was stirred at 80° C. for 16 hr. LCMS showed a peak (83%) with desired mass. The reaction mixture was dilute with water (20 mL) and extracted with EtOAc (20 mL×3). The combined organic phase was washed with brine (10 mL×2), dried over Na₂SO₄ and concentrated under reduced pressure. The residue was triturated with EtOAc and MeCN to afford tert-butyl N-[4-[[1-(2-fluoro-4-nitro-phenyl)-4-piperidyl]methyl-methyl-amino]cyclohexyl]carbamate (700 mg, 1.43 mmol, 75.91% yield, 95% purity) as yellow solid. MS(M+H)⁺=465.3.

¹H NMR (400 MHz, DMSO-d₆) δ=8.03-7.93 (m, 2H), 7.14 (t, J=9.2 Hz, 1H), 6.67 (br d, J=7.8 Hz, 1H), 3.68 (br d, J=12.5 Hz, 2H), 3.20-3.03 (m, 1H), 2.96-2.87 (m, 2H), 2.27-2.11 (m, 6H), 1.79 (br d, J=12.1 Hz, 4H), 1.72-1.58 (m, 3H), 1.36 (s, 9H), 1.29-1.08 (m, 6H).

### Step 2. Synthesis of tert-butyl N-[4-[[1-(4-amino-2-fluoro-phenyl)-4-piperidyl]methyl-methyl-amino]cyclohexyl]carbamate (4)

To a solution of tert-butyl N-[4-[[1-(2-fluoro-4-nitro-phenyl)-4-piperidyl]methyl-methyl-amino]cyclohexyl]carbamate (1.30 g, 2.80 mmol) in EtOH (15 mL) and H₂O (15 mL) were added Fe (468.81 mg, 8.39 mmol) and NH₄Cl (748.42 mg, 13.99 mmol). The mixture was stirred at 80° C. for 1 hr. LCMS showed a peak (67%) with the desired mass. The reaction mixture was filtered through a celite pad and concentrated under reduced pressure. The residue was diluted with NaHCO₃ solution (20 mL) and extracted with EtOAc (20 mL×3). The combined organic phase was concentrated under reduced pressure to afford tert-butyl N-[4-[[1-(4-amino-2-fluoro-phenyl)-4-piperidyl]methyl-methyl-amino]cyclohexyl]carb amate (800 mg, 1.53 mmol, 54.60% yield, 83% purity) as yellow solid. MS(M+H)⁺=435.3.

### Step 3. Synthesis of tert-butyl N-[4-[[1-[4-[(2,6-dioxo-3-piperidyl)amino]-2-fluoro-phenyl]-4-piperidyl]methyl-methyl-amino]cyclohexyl]carbamate (6)

To a solution of tert-butyl N-[4-[[1-(4-amino-2-fluoro-phenyl)-4-piperidyl]methyl-methyl-amino]cyclohexyl]carbamate (700.00 mg, 1.61 mmol) and 3-bromopiperidine-2, 6-dione (773.19 mg, 4.03 mmol) in MeCN (3.5 mL) was added NaHCO₃ (676.55 mg, 8.05 mmol). The mixture was stirred at 80° C. for 16 hr. LCMS showed a peak (67%) with the mass of desired product. The reaction mixture was diluted with MeCN (10 mL) and filtered. The filter cake was washed with water (10 mL), collected and dried to afford tert-butyl N-[4-[[1-[4-[(2,6-dioxo-3-piperidyl)amino]-2-fluoro-phenyl]-4-piperidyl]methyl-methylamino]cyclohexyl]carbamate (600 mg, 967.59 mol, 60.07% yield, 88% purity) as black solid. MS(M+H)⁺=546.3.

### Step 4. Synthesis of 3-[4-[4-[[(4-aminocyclohexyl)-methyl-amino]methyl]-1-piperidyl]-3-fluoro-anilino]piperidine-2,6-dione (7)

To a solution of tert-butyl N-[4-[[1-[4-[(2,6-dioxo-3-piperidyl) amino]-2-fluoro-phenyl]-4-piperidyl]methyl-methyl-amino]cyclohexyl]carbamate (600.00 mg, 1.10 mmol) in dioxane (5 mL) was added HCl/dioxane (4 M, 5 mL). The mixture was stirred at 20° C. for 1 hr. LCMS showed a peak (50%) with the mass of desired product. The reaction mixture was concentrated under reduced pressure to afford 3-[4-[4-[[(4-aminocyclohexyl)-methyl-amino]methyl]-1-piperidyl]-3-fluoro-anilino]piperidine-2, 6-dione (500 mg, 975.04 mol, 88.68% yield, 94% purity, HCl) as black solid. MS(M+H)⁺=446.3.

### Step 5. Synthesis of N-(2-chlorophenyl)-4-[[2-[4-[[4-[[1-[4-[(2,6-dioxo-3-piperidyl]amino]-2-fluoro-phenyl]-4-piperidyl]methyl-methyl-amino]cyclohexyl]carbamoyl]anilino]-5-fluoro-pyrimidin-4-yl]amino]benzamide (Compound 66)

To a solution of 3-[4-[4-[[(4-aminocyclohexyl)-methyl-amino]methyl]-1-piperidyl]-3-fluoro-anilino]piperidine-2, 6-dione (180 mg, 403.98 μmol) and 4-[[4-[4-[(2-chlorophenyl)carbamoyl]anilino]-5-fluoro-pyrimidin-2-yl]amino]benzoic acid (193.05 mg, 403.98 μmol) in DMF (0.5 mL) were added EDCI (116.16 mg, 605.96 μmol), HOBt (81.88 mg, 605.96 μmol) and DIPEA (156.63 mg, 1.21 mmol, 211.10 μL). The mixture was stirred at 20° C. for 16 hr. LCMS showed a peak (36%) with desired mass. The reaction mixture was diluted with water (10 mL) and filtered. The residue was purified by prep-HPLC (column: Phenomenex Luna C18 150*25 mm*10 um; mobile phase: [water (TFA)-ACN]; gradient: 20%-50% B over 10 min) and lyophilized to afford the impure product, which was further purified by prep-HPLC (column: Waters Xbridge 150*25 mm*5 um; mobile phase: [water (NH₄HCO₃)-ACN]; gradient: 16%-46% B over min) and lyophilized to afford N-(2-chlorophenyl)-4-[[2-[4-[[4-[[1-[4-[(2,6-dioxo-3-piperidyl)amino]-2-fluoro-phenyl]-4-piperidyl]methyl-methyl-amino]cyclohexyl]carbamoyl]anilino]-5-fluoro-pyrimidin-4-yl]amino]benzamide (25.6 mg, 27.71 mol, 6.86% yield, 98% purity) as white solid. MS(M+H)⁺=905.3.

¹H NMR (400 MHz, DMSO-d₆) δ=10.76 (s, 1H), 9.92 (s, 1H), 9.73 (s, 1H), 9.59 (s, 1H), 8.24 (d, J=3.4 Hz, 1H), 8.10-7.96 (m, 5H), 7.76 (s, 4H), 7.69-7.63 (m, 1H), 7.56 (dd, J=1.4, 8.0 Hz, 1H), 7.44-7.36 (m, 1H), 7.33-7.26 (m, 1H), 6.83 (br t, J=9.4 Hz, 1H), 6.55-6.37 (m, 2H), 5.84-5.71 (m, 1H), 4.30-4.20 (m, 1H), 3.78-3.58 (m, 1H), 3.18-3.08 (m, 2H), 2.30-2.15 (m, 6H), 2.13-2.02 (m, 2H), 1.94-1.69 (m, 8H), 1.51-1.17 (m, 9H).

### Example 67. Synthesis of N-(2-chlorophenyl)-4-((2-((4-((1-(1-(4-(4-(2,6-dioxopiperidin-3-yl)phenyl)piperazin-1-yl)propan-2-yl)piperidin-4-yl)carbamoyl)phenyl)amino)-5-fluoropyrimidin-4-yl) amino)benzamide (Compound 67)

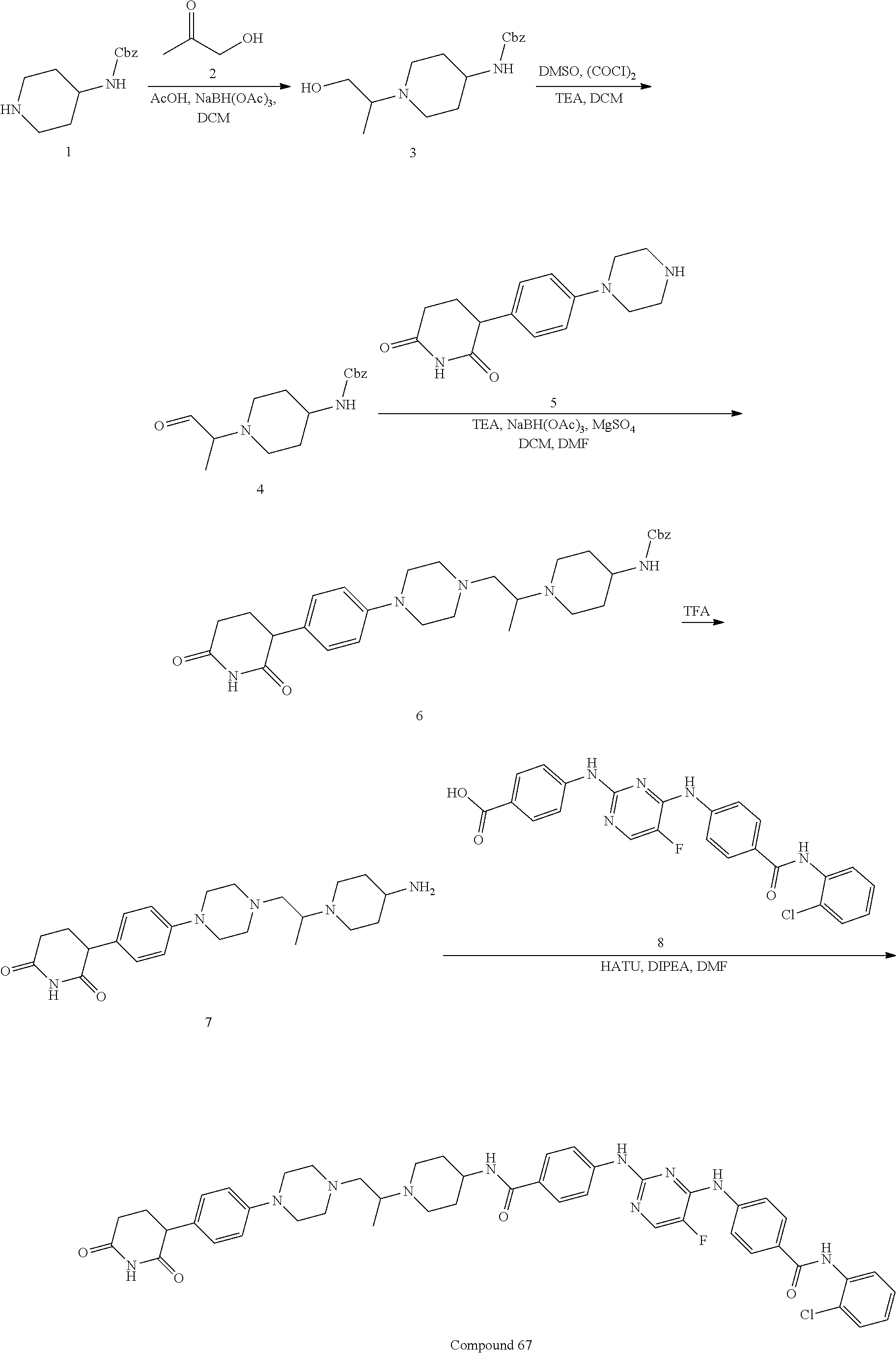

### Step 1. Synthesis of benzyl (1-(1-hydroxypropan-2-yl)piperidin-4-yl)carbamate (3)

To a solution of benzyl piperidin-4-ylcarbamate (850 mg, 3.63 mmol) and 1-hydroxypropan-2-one (1.26 g, 17.01 mmol, 1.17 mL) in DCM (9 mL) was added AcOH (254.86 mg, 4.24 mmol, 242.96 μL). After stirring at 25° C. for 0.5 hr, NaBH(OAc)₃ (2.7 g, 12.74 mmol) was added and the resulting mixture was stirred at 25° C. for 1 hr. LCMS showed benzyl piperidin-4-ylcarbamate was consumed completely and desired mass was detected. The reaction mixture was diluted with H₂O (50 mL) at 25° C., and then extracted with EtOAc (20 mL×2). The combined organic layers were dried over Na₂SO₄, filtered and concentrated under reduced pressure. The residue was purified by flash silica gel chromatography (ISCO®; 20 g SepaFlash® Silica Flash Column, Eluent of 0~20% MeOH/EtOAc @ 80 mL/min) to afford benzyl (1-(1-hydroxypropan-2-yl)piperidin-4-yl)carbamate (750 mg, 2.57 mmol, 70.71% yield) as a colourless oil. MS(M+H)⁺=293.1.

### Step 2. Synthesis of benzyl (1-(1-oxopropan-2-yl)piperidin-4-yl)carbamate (4)

To a solution of DMSO (250.67 mg, 3.21 mmol, 250.67 μL) in DCM (2 mL) was slowly added (COCl)₂ (193.62 mg, 1.53 mmol, 133.53 μL) at −70° C. and the mixture was stirred at −70° C. for 15 min. Then a solution of benzyl (1-(1-hydroxypropan-2-yl)piperidin-4-yl)carbamate (200 mg, 684.06 μmol) in DCM (2 mL) was slowly added. After stirring at −70° C. for 20 min, TEA (363.50 mg, 3.59 mmol, 0.5 mL) was added and the resulting mixture and stirred at −70° C. for 60 min. ¹H NMR showed desired compound was detected after work-up. The mixture was warmed to 20° C. and then quenched with water (20 mL). The mixture was extracted with DCM (10 mL×2), the combined organic layers were washed with water (10 mL×2), dried over Na₂SO₄ and filtered. The filtrate was concentrated under reduced pressure to afford benzyl (1-(1-oxopropan-2-yl)piperidin-4-yl)carbamate (260 mg, crude) as a colorless oil, which was used directly. MS(M+H)⁺=291.4.

### Step 3. Synthesis of benzyl (1-(1-(4-(4-(2,6-dioxopiperidin-3-yl)phenyl)piperazin-1-yl)propan-2-yl)piperidin-4-yl) carbamate (6)

To a solution of 3-(4-(piperazin-1-yl)phenyl)piperidine-2,6-dione (180 mg, 581.04 mol, HCl) in DCM (2 mL) were added TEA (293.97 mg, 2.91 mmol, 404.36 μL) and MgSO₄ (209.81 mg, 1.74 mmol). Then a solution of benzyl (1-(1-oxopropan-2-yl)piperidin-4-yl)carbamate (253.06 mg, 871.56 μmol) in DMF (2 mL) was added. After stirring at 25° C. for 0.5 hr, NaBH(OAc)₃ (369.44 mg, 1.74 mmol) was added and the resulting mixture was stirred at 25° C. for 1 hr. LCMS showed benzyl (1-(1-oxopropan-2-yl)piperidin-4-yl)carbamate was consumed completely. The reaction mixture was diluted with H₂O (50 mL) and extracted with EtOAc (20 mL×2). The combined organic layers were dried over Na₂SO₄, filtered and concentrated under reduced pressure. The residue was purified by flash silica gel chromatography (ISCO®; 12 g SepaFlash® Silica Flash Column, Eluent of 0~20% MeOH/EtOAc @ 50 mL/min) to afford benzyl (1-(1-(4-(4-(2,6-dioxopiperidin-3-yl)phenyl)piperazin-1-yl)propan-2-yl)piperidin-4-yl) carbamate (130 mg, 237.36 μmol, 40.85% yield) as a brown oil. MS(M+H)⁺=548.2.

### Step 4. Synthesis of 3-(4-(4-(2-(4-aminopiperidin-1-yl)propyl)piperazin-1-yl)phenyl)piperidine-2,6-dione (7)

A solution of benzyl (1-(1-(4-(4-(2,6-dioxopiperidin-3-yl)phenyl)piperazin-1-yl)propan-2-yl)piperidin-4-yl) carbamate (130 mg, 237.36 μmol) in TFA (2.00 g, 17.50 mmol, 1.3 mL) was stirred at 40° C. for 16 hr. LCMS showed benzyl (1-(1-(4-(4-(2,6-dioxopiperidin-3-yl)phenyl)piperazin-1-yl)propan-2-yl)piperidin-4-yl) carbamate was consumed completely and desired mass was detected. The reaction mixture was concentrated under reduced pressure to afford 3-(4-(4-(2-(4-aminopiperidin-1-yl)propyl)piperazin-1-yl)phenyl)piperidine-2,6-dione (125 mg, crude, TFA) as a brown oil. MS(M+H)⁺=414.2.

### Step 5. Synthesis of N-(2-chlorophenyl)-4-((2-((4-((1-(1-(4-(4-(2,6-dioxopiperidin-3-yl)phenyl)piperazin-1-yl)propan-2-yl)piperidin-4-yl)carbamoyl)phenyl)amino)-5-fluoropyrimidin-4-yl) amino)benzamide (Compound 67)

To a solution of 4-((4-((4-((2-chlorophenyl)carbamoyl)phenyl)amino)-5-fluoropyrimidin-2-yl)amino)benzoic acid (113.22 mg, 236.93 μmol) in DMF (1.3 mL) were added HATU (135.13 mg, 355.40 μmol) and DIPEA (153.11 mg, 1.18 mmol, 206.35 μL). After stirring at 25° C. for 0.5 hr, 3-(4-(4-(2-(4-aminopiperidin-1-yl)propyl)piperazin-1-yl)phenyl)piperidine-2,6-dione (125 mg, 236.93 mol, TFA) was added and the resulting mixture was stirred at 25° C. for 1 hr. LCMS showed 3-(4-(4-(2-(4-aminopiperidin-1-yl)propyl)piperazin-1-yl)phenyl)piperidine-2,6-dione was consumed completely and desired mass was detected. The reaction mixture was diluted with H₂O (50 mL) at 25° C., and then extracted with EtOAc (20 mL×2). The combined organic layers were washed with brine (20 mL), dried over Na₂SO₄, filtered and concentrated under reduced pressure. The residue was purified by prep-HPLC (column: Waters Xbridge 150*25 mm*5 um; mobile phase: [water (NH₄HCO₃)-ACN]; gradient: 36%-66% B over min) and lyophilized to afford N-(2-chlorophenyl)-4-((2-((4-((1-(1-(4-(4-(2,6-dioxopiperidin-3-yl)phenyl)piperazin-1-yl)propan-2-yl)piperidin-4-yl)carbamoyl)phenyl)amino)-5-fluoropyrimidin-4-yl)amino)benzamide (36.6 mg, 39.39 μmol, 16.63% yield, 94% purity) as a white solid. MS(M+H)⁺=873.4.

¹H NMR (400 MHz, DMSO-d₆) δ=10.77 (s, 1H), 9.92 (s, 1H), 9.73 (s, 1H), 9.59 (s, 1H), 8.24 (d, J=3.5 Hz, 1H), 8.08-7.95 (m, 5H), 7.78-7.75 (m, 4H), 7.65 (d, J=6.8 Hz, 1H), 7.60-7.53 (m, 1H), 7.40 (t, J=7.5 Hz, 1H), 7.34-7.25 (m, 1H), 7.04 (d, J=8.6 Hz, 2H), 6.88 (d, J=8.6 Hz, 2H), 3.77-3.68 (m, 2H), 3.17-3.05 (m, 4H), 2.88-2.73 (m, 3H), 2.70-2.66 (m, 1H), 2.65-2.58 (m, 3H), 2.50-2.44 (m, 2H), 2.40-2.35 (m, 1H), 2.36-2.24 (m, 3H), 2.20-2.07 (m, 1H), 2.05-1.95 (m, 1H), 1.82-1.71 (m, 2H), 1.60-1.43 (m, 2H), 0.96 (br d, J=6.5 Hz, 3H).

### Example 68. Synthesis of N-(2-chlorophenyl)-4-((2-((4-(((3S)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl) amino)benzamide (Compound 68)

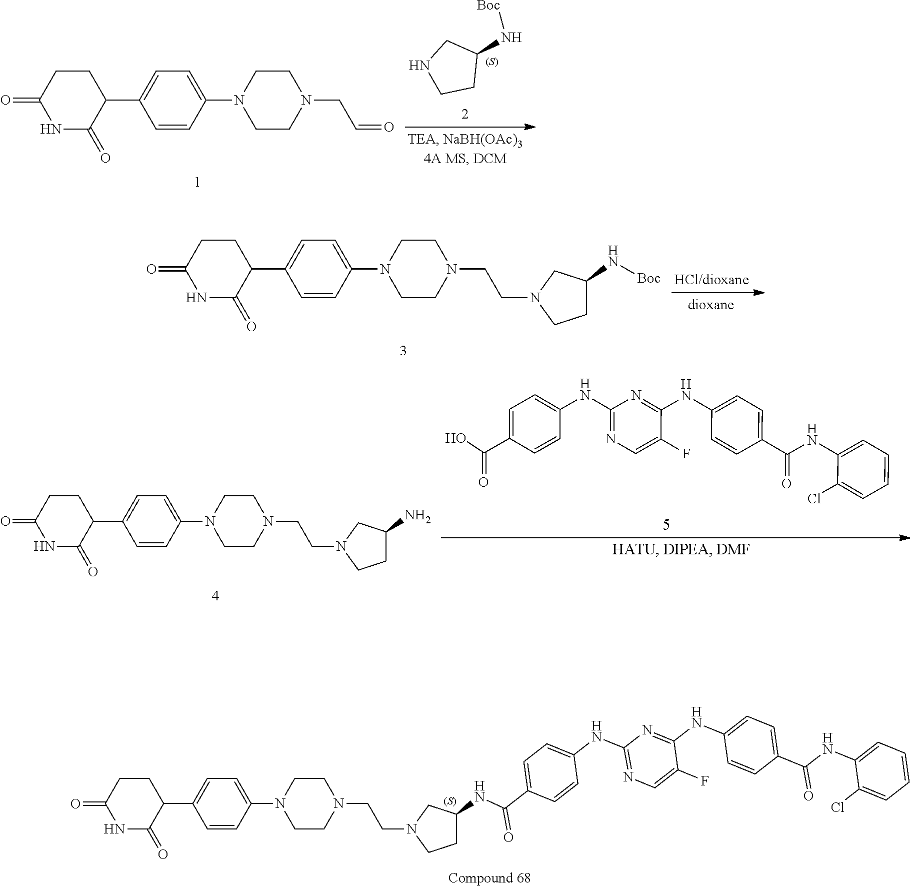

### Step 1. Synthesis of tert-butyl ((3S)-1-(2-(4-(4-(2,6-dioxopiperidin-3-yl)phenyl)piperazin-1-yl)ethyl)pyrrolidin-3-yl)carbamate (3)

To a solution of 2-(4-(4-(2,6-dioxopiperidin-3-yl)phenyl)piperazin-1-yl)acetaldehyde (120 mg, 380.51 μmol) and tert-butyl (S)-pyrrolidin-3-ylcarbamate (92.13 mg, 494.66 μmol) in DCM (4 mL) was added TEA (38.50 mg, 380.51 μmol, 52.96 μL) and 4 Å MS (20 mg). After half an hour, NaBH(OAc)₃ (241.94 mg, 1.14 mmol) was added to the reaction mixture. The mixture was stirred at 20° C. for 16 hr. LCMS showed 2-(4-(4-(2,6-dioxopiperidin-3-yl)phenyl)piperazin-1-yl)acetaldehyde was consumed completely and one main peak with desired mass was detected. The residue was diluted with NaHCO₃ solution (50 mL) and extracted with DCM (20 mL×3). The combined organic layers were washed with water (20 mL), dried over Na₂SO₄, filtered and concentrated under reduced pressure. The residue was purified by column chromatography by prep-TLC (SiO₂, EtOAc:MeOH=3:1) to afford tert-butyl ((3S)-1-(2-(4-(4-(2,6-dioxopiperidin-3-yl)phenyl)piperazin-1-yl)ethyl)pyrrolidin-3-yl)carbamate (90 mg, 185.33 mol, 48.71% yield) as a white solid. MS(M+H)⁺=486.4.

### Step 2. Synthesis of 3-(4-(4-(2-((S)-3-aminopyrrolidin-1-yl)ethyl)piperazin-1-yl)phenyl)piperidine-2,6-dione (4)

To a solution of tert-butyl ((3S)-1-(2-(4-(4-(2,6-dioxopiperidin-3-yl)phenyl)piperazin-1-yl)ethyl)pyrrolidin-3-yl)carbamate (90 mg, 185.33 μmol) in dioxane (1 mL) was added HCl/dioxane (4 M, 1 mL). The mixture was stirred at 20° C. for 1 hr. LCMS showed one main peak with desired mass was detected. The reaction mixture was concentrated under reduced pressure to remove solvent to afford 3-(4-(4-(2-((S)-3-aminopyrrolidin-1-yl)ethyl)piperazin-1-yl)phenyl)piperidine-2,6-dione (100 mg, crude, HCl salt) as a white solid. MS(M+H)⁺=386.2.

### Step 3. Synthesis of N-(2-chlorophenyl)-4-((2-((4-(((3S)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl) amino)benzamide (Compound 68)

To a solution of 4-((4-((4-((2-chlorophenyl)carbamoyl)phenyl)amino)-5-fluoropyrimidin-2-yl)amino)benzoic acid (90 mg, 188.33 μmol) and 3-(4-(4-(2-((S)-3-aminopyrrolidin-1-yl)ethyl)piperazin-1-yl)phenyl)piperidine-2,6-dione (95.36 mg, 226.00 mol, HCl salt) in DMF (2 mL) was added HATU (107.42 mg, 282.50 μmol) and DIPEA (121.70 mg, 941.67 μmol, 164.02 μL). The mixture was stirred at 20° C. for 2 hr. LCMS showed one main peak with desired mass was detected. The reaction mixture was diluted with water (20 mL) and extracted with THF (10 mL×3). The combined organic layers were washed with brine (10 mL×3), dried over Na₂SO₄, filtered and concentrated under reduced pressure. The residue was purified by prep-HPLC (column: Waters Xbridge C18 150×50 mm×10 μm; mobile phase: [water(NH₄HCO₃)-ACN]; gradient: 29%-59% B over 10 min) and lyophilized to afford N-(2-chlorophenyl)-4-((2-((4-(((3S)-1-(2-(4-(4-(2,6-dioxopiperidin-3-yl)phenyl)piperazin-1-yl)ethyl)pyrrolidin-3-yl)carbamoyl)phenyl)amino)-5-fluoropyrimidin-4-yl)amino)benzamide (21.8 mg, 23.21 mol, 12.32% yield, 90% purity) as a light yellow solid. MS(M+H)⁺=845.3.

¹H NMR (400 MHz, DMSO-d₆) δ=10.76 (s, 1H), 9.92 (s, 1H), 9.74 (s, 1H), 9.61 (s, 1H), 8.25 (d, J=3.5 Hz, 1H), 8.20 (br d, J=6.5 Hz, 1H), 8.05-7.99 (m, 4H), 7.81-7.76 (m, 4H), 7.66 (dd, J=1.3, 7.9 Hz, 1H), 7.56 (dd, J=1.3, 8.0 Hz, 1H), 7.42-7.37 (m, 1H), 7.32-7.27 (m, 1H), 7.03 (d, J=8.6 Hz, 2H), 6.87 (d, J=8.8 Hz, 2H), 4.41-4.30 (m, 1H), 3.71 (dd, J=5.0, 11.0 Hz, 1H), 3.15-3.05 (m, 4H), 2.83-2.75 (m, 1H), 2.66-2.60 (m, 2H), 2.59-2.55 (m, 2H), 2.55-2.53 (m, 4H), 2.48-2.40 (m, 5H), 2.15-2.06 (m, 2H), 2.03-1.97 (m, 1H), 1.81-1.71 (m, 1H).

### Example 69. Synthesis of N-(2-chlorophenyl)-4-((2-((4-(((1r,4r)-4-(((1-(4-((2,6-dioxopiperidin-3-yl)amino)phenyl)piperidin-4-yl)methyl)(methyl)amino)cyclohexyl)carbamoyl)phenyl) amino)-5-fluoropyrimidin-4-yl)amino)benzamide (Compound 69)

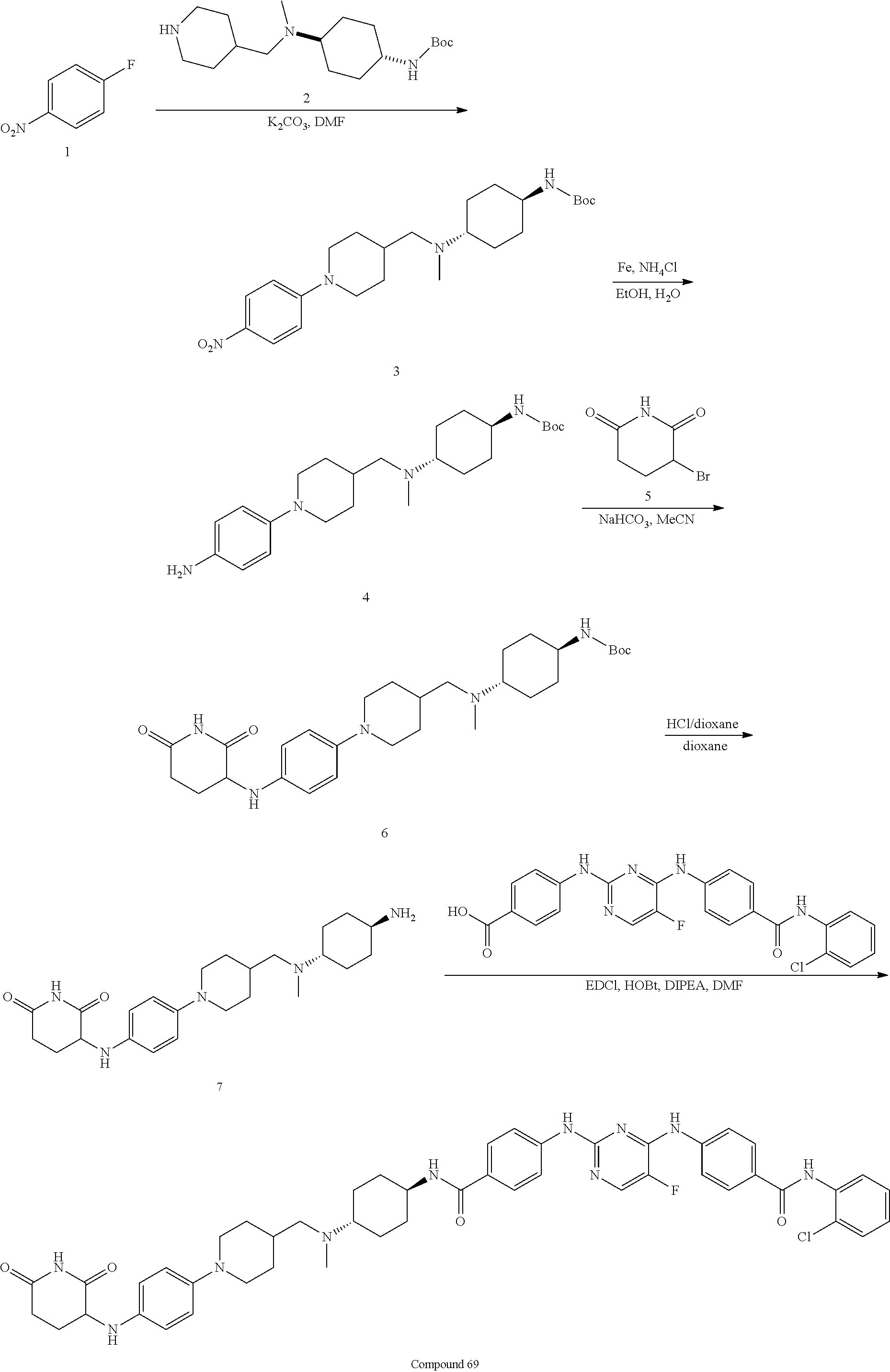

### Step 1. Synthesis of tert-butyl N-[4-[methyl-[[1-(4-nitrophenyl)-4-piperidyl]methyl]amino]cyclohexyl]carbamate (3)

To a solution of 1-fluoro-4-nitro-benzene (400 mg, 2.83 mmol, 300.75 μL) and tert-butyl N-[4-[methyl (4-piperidylmethyl) amino]cyclohexyl]carbamate (922.72 mg, 2.83 mmol) in DMF (5 mL) was added K₂CO₃ (1.18 g, 8.50 mmol). The mixture was stirred at 80° C. for 16 hr. LCMS showed a peak (56%) with the mass of desired product. The reaction mixture was diluted with water (20 mL) and extracted with EtOAc (20 mL×3). The combined organic phase was washed with brine (10 mL×2), dried over Na₂SO₄ and concentrated under reduced pressure to afford a residue. The residue was triturated with EtOAc (5 mL) and MeCN (5 mL) to afford tert-butyl N-[4-[methyl-[[1-(4-nitrophenyl)-4-piperidyl]methyl]amino]cyclohexyl]carbamate (640 mg, 1.43 mmol, 50.55% yield, 100% purity) as yellow solid. MS(M+H)⁺=447.3.

### Step 2. Synthesis of tert-butyl N-[4-[[1-(4-aminophenyl)-4-piperidyl]methyl-methyl-amino]cyclohexyl]carbamate (4)

To a solution of tert-butyl N-[4-[methyl-[[1-(4-nitrophenyl)-4-piperidyl]methyl]amino]cyclohexyl]carbamate (640 mg, 1.43 mmol) in EtOH (5 mL) and H₂O (5 mL) were added Fe (240.10 mg, 4.30 mmol) and NH₄Cl (383.29 mg, 7.17 mmol). The mixture was stirred at 80° C. for 1.5 hr. LCMS showed a peak (70%) with the mass of desired product. The reaction mixture was filtered through a celite pad and concentrated under reduced pressure to remove ethanol. The residue was diluted with aqueous NaHCO₃ solution (30 mL) and extracted with EtOAc (10 mL×2). The combined organic phase was dried over Na₂SO₄ and concentrated under reduced pressure to afford tert-butyl N-[4-[[1-(4-aminophenyl)-4-piperidyl]methyl-methyl-amino]cyclohexyl]carbamate (400 mg, 864.14 μmol, 60.30% yield, 90% purity) as yellow solid. MS(M+H)⁺=417.3.

### Step 3. Synthesis of tert-butyl N-[4-[[1-[4-[(2, 6-dioxo-3-piperidyl)amino]phenyl]-4-piperidyl]methyl-methyl-amino]cyclohexyl]carbamate (6)

To a solution of tert-butyl N-[4-[[1-(4-aminophenyl)-4-piperidyl]methyl-methyl-amino]cyclohexyl]carbamate (400 mg, 960.16 μmol) and 3-bromopiperidine-2, 6-dione (460.90 mg, 2.40 mmol) in MeCN (2 mL) was added NaHCO₃ (403.30 mg, 4.80 mmol). The mixture was stirred at 80° C. for 16 hr. LCMS showed a peak (26%) with desired mass. The reaction mixture was filtered and concentrated under reduced pressure to afford a residue, which was purified by flash silica gel chromatography (12 g SepaFlash® Silica Flash Column, Eluent of 0-15% MeOH/EtOAc gradient @ 60 mL/min) to afford tert-butyl N-[4-[[1-[4-[(2,6-dioxo-3-piperidyl)amino]phenyl]-4-piperidyl]methyl-methyl-amino]cyclohexyl]carbamate (500 mg, 577.98 mol, 60.20% yield, 61% purity) as black oil. MS(M+H)⁺=528.3.

### Step 4. Synthesis of 3-[4-[4-[[(4-aminocyclohexyl)-methyl-amino]methyl]-1-piperidyl]anilino]piperidine-2,6-dione (7)

To a solution of tert-butyl N-[4-[[1-[4-[(2,6-dioxo-3-piperidyl) amino]phenyl]-4-piperidyl]methyl-methyl-amino]cyclohexyl]carbamate (500 mg, 947.51 μmol) in dioxane (5 mL) was added HCl/dioxane (4 M, 5 mL). The mixture was stirred at 20° C. for 1 hr. LCMS showed a peak (85%) with the mass of desired product. The reaction mixture was concentrated under reduced pressure to afford 3-[4-[4-[[(4-aminocyclohexyl)-methyl-amino]methyl]-1-piperidyl]anilino]piperidine-2, 6-dione (400 mg, 749.93 mol, 79.15% yield, 87% purity, HCl) as gray solid. MS(M+H)⁺=428.2.

### Step 5. Synthesis of N-(2-chlorophenyl)-4-((2-((4-(((1r,4r)-4-(((1-(4-((2,6-dioxopiperidin-3-ylamino)phenyl) piperidin-4-yl)methyl)(methyl)amino)cyclohexyl)carbamoyl)phenyl)amino)-5-fluoropyrimidin-4-yl)amino)benzamide (Compound 69)

To a solution of 3-((4-(4-((((1r,4r)-4-aminocyclohexyl)(methyl)amino)methyl)piperidin-1-yl)phenyl)amino)piperidine-2,6-dione (400 mg, 935.49 μmol) and 4-[[4-[4-[(2-chlorophenyl) carbamoyl]anilino]-5-fluoro-pyrimidin-2-yl]amino]benzoic acid (268.23 mg, 561.30 mol) in DMF (3 mL) were added EDCI (269.00 mg, 1.40 mmol), HOBt (126.41 mg, 935.49 μmol) and DIPEA (362.72 mg, 2.81 mmol, 488.84 μL). The mixture was stirred at 20° C. for 1 hr. LCMS showed a peak (72%) with mass of desired product. The reaction mixture was diluted with water (30 mL) and filtered to afford a residue. The residue was purified by prep-HPLC (column: Phenomenex luna C18 150*40 mm*15 um; mobile phase: [water (TFA)-ACN]; gradient: 10%-40% B over 10 min) and lyophilized to afford the impure product, which was further purified by prep-HPLC (column: Waters Xbridge 150*25 mm*5 um; mobile phase: [water (NH₄HCO₃)-ACN]; gradient: 34%-64% B over 10 min) and lyophilized to afford N-(2-chlorophenyl)-4-((2-((4-(((1r,4r)-4-(((1-(4-((2,6-dioxopiperidin-3-yl)amino)phenyl) piperidin-4-yl)methyl)(methyl)amino)cyclohexyl)carbamoyl)phenyl)amino)-5-fluoropyrimidin-4-yl)amino)benzamide (112.9 mg, 123.40 mol, 13.19% yield, 97% purity) as white solid. MS M+H)+=887.4.

¹H NMR (400 MHz, DMSO-d₆) δ=10.75 (s, 1H), 9.92 (s, 1H), 9.73 (s, 1H), 9.59 (s, 1H), 8.24 (d, J=3.4 Hz, 1H), 8.06-7.97 (m, 4H), 7.94 (br d, J=8.1 Hz, 1H), 7.76 (s, 4H), 7.65 (dd, J=1.3, 7.9 Hz, 1H), 7.56 (dd, J=1.4, 8.0 Hz, 1H), 7.44-7.36 (m, 1H), 7.34-7.25 (m, 1H), 6.75 (d, J=9.0 Hz, 2H), 6.60 (d, J=8.8 Hz, 2H), 5.34 (d, J=7.3 Hz, 1H), 4.25-4.12 (m, 1H), 3.77-3.62 (m, 1H), 3.39-3.36 (m, 2H), 2.65-2.55 (m, 2H), 2.46-2.36 (m, 2H), 2.31-2.22 (m, 3H), 2.18 (s, 3H), 2.15-2.06 (m, 1H), 1.92-1.80 (m, 3H), 1.80-1.68 (m, 4H), 1.50-1.40 (m, 1H), 1.36-1.13 (m, 6H).

### Example 70. Synthesis of N-(4-((4-((4-((2-chlorophenyl)carbamoyl)phenyl)amino)-5-fluoropyrimidin-2-yl)amino)phenyl)-1-(2-(4-(4-(2,6-dioxopiperidin-3-yl)phenyl)piperazin-1-yl)ethyl)piperidine-4-carboxamide (Compound 70)

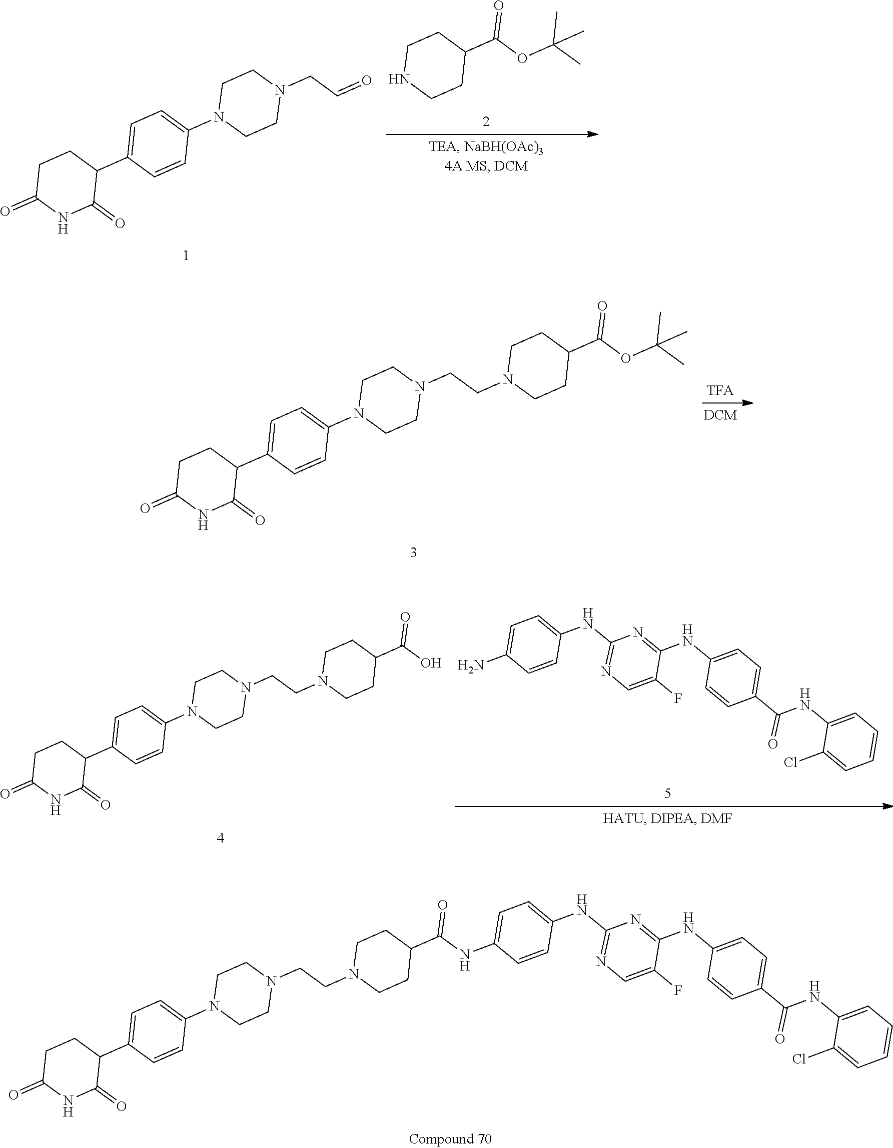

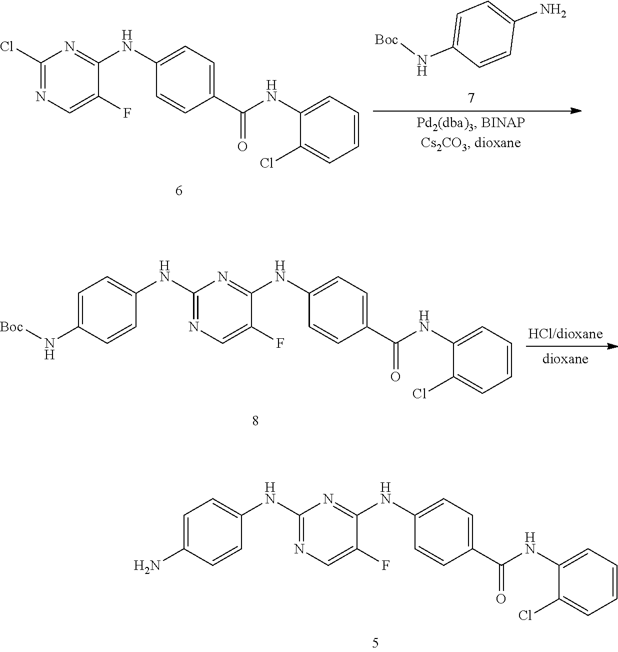 [SMILES: O=C(Nc1ccccc1Cl)c1ccc(Nc2nc(Cl)ncc2F)cc1 ; CC(C)(C)OC(=O)Nc1ccc(N)cc1 ; CC(C)(C)OC(=O)Nc1ccc(Nc2ncc(F)c(Nc3ccc(C(=O)Nc4ccccc4Cl)cc3)n2)cc1 ; Nc1ccc(Nc2ncc(F)c(Nc3ccc(C(=O)Nc4ccccc4Cl)cc3)n2)cc1]

### Step 1. Synthesis of tert-butyl 1-(2-(4-(4-(2,6-dioxopiperidin-3-yl)phenyl)piperazin-1-yl)ethyl)piperidine-4-carboxylate (3)

To a solution of 2-(4-(4-(2,6-dioxopiperidin-3-yl)phenyl)piperazin-1-yl)acetaldehyde (120 mg, 380.51 μmol) and tert-butyl piperidine-4-carboxylate (91.64 mg, 494.66 mol) in DCM (4 mL) was added TEA (38.50 mg, 380.51 μmol, 52.96 μL) and 4 Å MS (20 mg). After stirring at 20° C. for 30 min, NaBH(OAc)₃ (241.94 mg, 1.14 mmol) was added and the resulting mixture was stirred at 20° C. for 16 hr. LCMS showed one main peak with desired mass. The residue was diluted with NaHCO₃ solution (20 mL) and extracted with DCM (20 mL×3). The combined organic layers were washed with water (20 mL), dried over Na₂SO₄, filtered and concentrated under reduced pressure. The residue was purified by column chromatography by prep-TLC (SiO₂, EtOAc:MeOH=5:1) to afford tert-butyl 1-(2-(4-(4-(2,6-dioxopiperidin-3-yl)phenyl)piperazin-1-yl)ethyl)piperidine-4-carboxylate (90 mg, 185.71 mol, 48.81% yield) as a white solid. MS(M+H)⁺=485.4.

### Step 2. Synthesis of 1-(2-(4-(4-(2,6-dioxopiperidin-3-yl)phenyl)piperazin-1-yl)ethyl)piperidine-4-carboxylic acid (4)

To a solution of tert-butyl 1-(2-(4-(4-(2,6-dioxopiperidin-3-yl)phenyl)piperazin-1-yl)ethyl)piperidine-4-carboxylate (90 mg, 185.71 μmol) in DCM (3 mL) was added TFA (1.54 g, 13.46 mmol, 1 mL). The mixture was stirred at 20° C. for 2 hr. LCMS showed the starting material was consumed completely. The reaction mixture was concentrated under reduced pressure to afford 1-(2-(4-(4-(2,6-dioxopiperidin-3-yl)phenyl)piperazin-1-yl)ethyl)piperidine-4-carboxylic acid (110 mg, crude, TFA salt) as a yellow oil. MS(M+H)⁺=429.2.

### Step 3. Synthesis of tert-butyl (4-((4-((4-((2-chlorophenyl)carbamoyl)phenyl)amino)-5-fluoropyrimidin-2-yl)amino)phenyl)carbamate (8)

To a solution of 4-((2-chloro-5-fluoropyrimidin-4-yl)amino)-N-(2-chlorophenyl)benzamide (10.00 g, 26.51 mmol) and tert-butyl (4-aminophenyl)carbamate (5.52 g, 26.51 mmol) in dioxane (120 mL) was added Cs₂CO₃ (25.91 g, 79.53 mmol), Pd₂(dba)₃ (2.43 g, 2.65 mmol) and BINAP (3.30 g, 5.30 mmol) under N₂. The mixture was stirred at 95° C. for 16 hr under N₂. LCMS showed ~28% of 4-((2-chloro-5-fluoropyrimidin-4-yl)amino)-N-(2-chlorophenyl)benzamide remained and ~16% of desired compound was detected. The reaction mixture was concentrated under reduced pressure to remove solvent. The residue was diluted with water (100 mL) and extracted with EtOAc (30 mL×3). The combined organic layers were washed with water (50 mL), dried over Na₂SO₄, filtered and concentrated under reduced pressure. The residue was purified by flash silica gel chromatography (ISCO®; 120 g SepaFlash® Silica Flash Column, Eluent of 50-60% EtOAc/Petroleum ether gradient @ 80 mL/min). The crude product was triturated with EtOAc (80 mL) at 25° C. for 30 min to afford tert-butyl (4-((4-((4-((2-chlorophenyl) carbamoyl)phenyl)amino)-5-fluoropyrimidin-2-yl)amino)phenyl)carbamate (9.5 g, crude) as a yellow solid. MS(M+H)⁺=549.2.

### Step 4. Synthesis of 4-((2-((4-aminophenyl)amino)-5-fluoropyrimidin-4-yl)amino)-N-(2-chlorophenyl)benzamide (5)

To a solution of tert-butyl (4-((4-((4-((2-chlorophenyl)carbamoyl)phenyl)amino)-5-fluoropyrimidin-2-yl)amino)phenyl)carbamate (2 g, 3.64 mmol) in dioxane (20 mL) was added HCl/dioxane (4 M, 20 mL). The mixture was stirred at 20° C. for 6 hr. LCMS showed one main peak with desired mass. The reaction mixture was concentrated under reduced pressure to remove solvent. The residue was diluted with water (50 mL) and adjusted pH=8 by NaHCO₃ aq at 0° C. and extracted with EtOAc (50 mL×3). The combined organic layers were washed with water (50 mL), dried over Na₂SO₄, filtered and concentrated under reduced pressure to give a residue, which was purified by flash silica gel chromatography (ISCO®; 40 g SepaFlash® Silica Flash Column, Eluent of 50-80% EtOAc/Petroleum ether gradient @ 80 mL/min) to afford 4-((2-((4-aminophenyl)amino)-5-fluoropyrimidin-4-yl)amino)-N-(2-chlorophenyl)benzamide (1.3 g, 2.90 mmol, 79.50% yield) as a yellow solid. MS(M+H)⁺=449.2.

### Step 5. Synthesis of N-(4-((4-((4-((2-chlorophenyl)carbamoyl)phenyl)amino)-5-fluoropyrimidin-2-yl)amino)phenyl)-1-(2-(4-(4-(2,6-dioxopiperidin-3-yl)phenyl)piperazin-1-yl)ethyl)piperidine-4-carboxamide (Compound 70)

To a solution of 4-((2-((4-aminophenyl)amino)-5-fluoropyrimidin-4-yl)amino)-N-(2-chlorophenyl) benzamide (90 mg, 200.50 μmol) and 1-(2-(4-(4-(2,6-dioxopiperidin-3-yl)phenyl)piperazin-1-yl)ethyl)piperidine-4-carboxylic acid (108.78 mg, 200.50 mol, TFA salt) in DMF (2 mL) was added HATU (114.35 mg, 300.75 μmol) and DIPEA (129.56 mg, 1.00 mmol, 174.61 μL). The mixture was stirred at 20° C. for 2 hr. LCMS showed 4-((2-((4-aminophenyl)amino)-5-fluoropyrimidin-4-yl)amino)-N-(2-chlorophenyl)benzamide was consumed completely and one main peak with desired mass was detected. The reaction mixture was diluted with water (30 mL) and extracted with THF (20 mL×3). The combined organic layers were washed with brine (20 mL×3), dried over Na₂SO₄, filtered and concentrated under reduced pressure to give a residue. The residue was purified by prep-HPLC (column: Waters Xbridge C18 150×50 mm×10 μm; mobile phase: [water(NH₄HCO₃)-ACN]; gradient: 30%-60% B over 10 min). The residue was purified by prep-HPLC (column: Phenomenex Luna C18 150×25 mm×10 μm; mobile phase: [water(TFA)-ACN]; gradient: 19%-49% B over 10 min) and lyophilized to afford N-(4-((4-((4-((2-chlorophenyl)carbamoyl)phenyl)amino)-5-fluoropyrimidin-2-yl)amino)phenyl)-1-(2-(4-(4-(2,6-dioxopiperidin-3-yl)phenyl)piperazin-1-yl)ethyl)piperidine-4-carboxamide (29.0 mg, 20.96 mol, 10.46% yield, 95.1% purity, 4TFA) as a white solid. MS(M+H)⁺=859.4.

¹H NMR (400 MHz, MeOD) δ=8.06 (d, J=4.9 Hz, 1H), 7.99-7.94 (m, 2H), 7.92-7.84 (m, 2H), 7.77 (dd, J=1.5, 7.9 Hz, 1H), 7.63-7.57 (m, 2H), 7.53 (dd, J=1.4, 8.0 Hz, 1H), 7.48-7.42 (m, 2H), 7.41-7.35 (m, 1H), 7.29-7.24 (m, 1H), 7.19 (d, J=8.7 Hz, 2H), 7.03 (d, J=8.8 Hz, 2H), 3.84-3.78 (m, 1H), 3.69-3.60 (m, 2H), 3.43-3.39 (m, 2H), 3.39-3.35 (m, 4H), 3.22-3.10 (m, 4H), 3.10-3.03 (m, 4H), 2.77-2.65 (m, 2H), 2.65-2.57 (m, 1H), 2.24-2.16 (m, 2H), 2.16-2.03 (m, 4H).

### Example 71. Synthesis of N-(2-chlorophenyl)-4-((2-((4-((1-(3-(4-(4-(2,6-dioxopiperidin-3-yl)phenyl)piperazin-1-yl)propyl)piperidin-4-yl)carbamoyl)phenyl)amino)-5-fluoropyrimidin-4-yl) amino)benzamide (Compound 71)

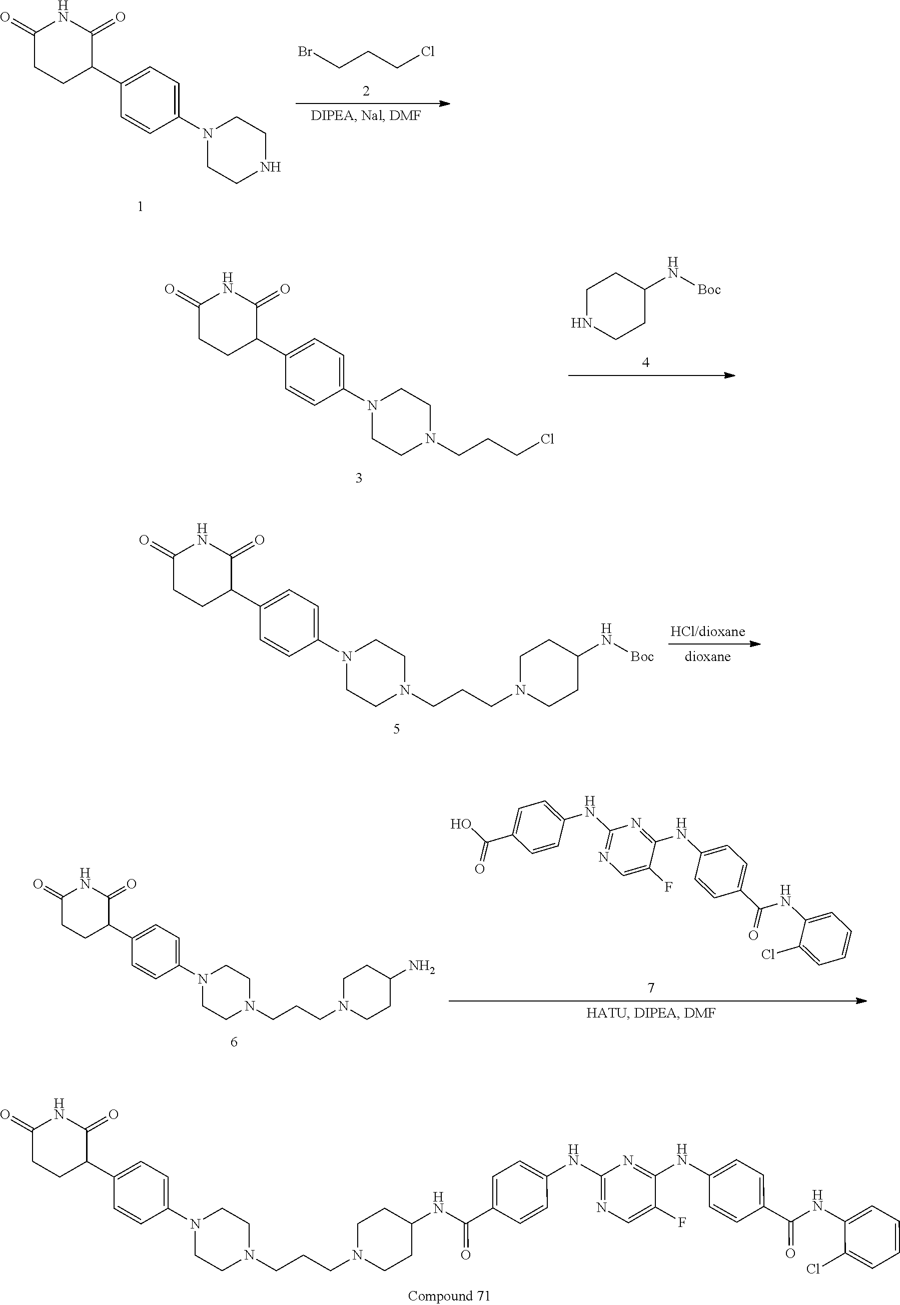

### Step 1. Synthesis of 3-(4-(4-(3-chloropropyl)piperazin-1-yl)phenyl)piperidine-2,6-dione (3)

To a solution of 3-(4-(piperazin-1-yl)phenyl)piperidine-2,6-dione (400 mg, 1.29 mmol, HCl salt) and 1-bromo-3-chloropropane (223.61 mg, 1.42 mmol, 140.11 μL) in DMF (5 mL) were added DIPEA (500.62 mg, 3.87 mmol, 674.69 μL) and NaI (19.35 mg, 129.12 μmol). The mixture was stirred at 80° C. for 2 hr. LCMS showed a main peak with desired mass. The desired product 3-(4-(4-(3-chloropropyl)piperazin-1-yl)phenyl)piperidine-2,6-dione (451 mg, crude) was obtained as reaction mixture and used for the next step directly. MS(M+H)⁺=350.1.

### Step 2. Synthesis of tert-butyl (1-(3-(4-(4-(2,6-dioxopiperidin-3-yl)phenyl)piperazin-1-yl)propyl)piperidin-4-yl)carbamate (5)

To a solution of 3-(4-(4-(3-chloropropyl)piperazin-1-yl)phenyl)piperidine-2,6-dione (451.7 mg, 1.29 mmol)(reaction mixture from previous step used directly) was added tert-butyl piperidin-4-ylcarbamate (310.30 mg, 1.55 mmol). The mixture was stirred at 80° C. for 16 hr. LCMS showed 3-(4-(4-(3-chloropropyl)piperazin-1-yl)phenyl)piperidine-2,6-dione was consumed completely and one main peak with desired mass was detected. The mixture was diluted with water (30 mL) and extracted with EtOAc (10 mL×3). The combined organic layers were washed with brine (10 mL×3), dried over Na₂SO₄, filtered and concentrated under reduced pressure. The residue was purified by flash silica gel chromatography (ISCO®; 20 g SepaFlash® Silica Flash Column, Eluent of 80-100% DCM/MeOH gradient @ 80 mL/min) to afford tert-butyl (1-(3-(4-(4-(2,6-dioxopiperidin-3-yl)phenyl)piperazin-1-yl)propyl)piperidin-4-yl)carbamate (300 mg, 432.18 mol, 33.47% yield, 74% purity) as a yellow solid. MS(M+H)⁺=514.4.

### Step 3. Synthesis of 3-(4-(4-(3-(4-aminopiperidin-1-yl)propyl)piperazin-1-yl)phenyl)piperidine-2,6-dione (6)

To a solution of tert-butyl (1-(3-(4-(4-(2,6-dioxopiperidin-3-yl)phenyl)piperazin-1-yl)propyl)piperidin-4-yl)carbamate (300 mg, 432.18 μmol) in dioxane (3 mL) was added HCl/dioxane (4 M, 3 mL). The mixture was stirred at 20° C. for hr. LCMS showed the starting material was consumed completely. The reaction mixture was concentrated under reduced pressure to afford 3-(4-(4-(3-(4-aminopiperidin-1-yl)propyl)piperazin-1-yl)phenyl)piperidine-2,6-dione (200 mg, crude, HCl salt) as a white solid. MS(M+H)⁺=414.3.

### Step 4. Synthesis of N-(2-chlorophenyl)-4-((2-((4-((1-(3-(4-(4-(2,6-dioxopiperidin-3-yl)phenyl)piperazin-1-yl)propyl)piperidin-4-yl)carbamoyl)phenyl)amino)-5-fluoropyrimidin-4-yl) amino)benzamide (Compound 71)

To a solution of 4-((4-((4-((2-chlorophenyl)carbamoyl)phenyl)amino)-5-fluoropyrimidin-2-yl)amino)benzoic acid (150 mg, 313.89 μmol) and 3-(4-(4-(3-(4-aminopiperidin-1-yl)propyl)piperazin-1-yl)phenyl)piperidine-2,6-dione (169.51 mg, 376.67 μmol, HCl salt) in DMF (2 mL) were added HATU (179.03 mg, 470.84 μmol) and DIPEA (121.70 mg, 941.67 μmol, 164.02 μL). The mixture was stirred at 20° C. for 2 hr. LCMS showed one main peak with desired mass was detected. The residue was diluted with water (20 mL) and extracted with EtOAc (10 mL×3). The combined organic layers were washed with brine (10 mL×3), dried over Na₂SO₄, filtered and concentrated under reduced pressure. The residue was purified by prep-HPLC (column: Phenomenex luna C18 150×25 mm×10 μm; mobile phase: [water(FA)-ACN]; gradient: 14%-47% B over 11 min) to afford the impure product, which was further purified by prep-HPLC (column: Waters Xbridge C18 150×50 mm×10 μm; mobile phase: [water(NH₄HCO₃)-ACN]; gradient: 30%-60% B over 10 min) and lyophilized to afford N-(2-chlorophenyl)-4-((2-((4-((1-(3-(4-(4-(2,6-dioxopiperidin-3-yl)phenyl)piperazin-1-yl)propyl)piperidin-4-yl)carbamoyl)phenyl)amino)-5-fluoropyrimidin-4-yl)amino)benzamide (22.9 mg, 25.85 mol, 8.24% yield, 98.6% purity) as a white solid. MS(M+H)⁺=873.4.

¹H NMR (400 MHz, DMSO-d₆) δ=10.77 (br s, 1H), 9.92 (s, 1H), 9.74 (s, 1H), 9.60 (s, 1H), 8.24 (d, J=3.4 Hz, 1H), 8.07-7.96 (m, 5H), 7.81-7.72 (m, 4H), 7.65 (br d, J=7.8 Hz, 1H), 7.56 (br d, J=7.4 Hz, 1H), 7.40 (br t, J=8.0 Hz, 1H), 7.33-7.26 (m, 1H), 7.04 (br d, J=8.4 Hz, 2H), 6.88 (br d, J=8.4 Hz, 2H), 3.72 (br dd, J=4.7, 11.2 Hz, 2H), 3.14-3.07 (m, 4H), 2.90-2.82 (m, 2H), 2.68-2.58 (m, 3H), 2.46-2.41 (m, 2H), 2.38-2.24 (m, 5H), 2.18-2.10 (m, 1H), 2.02-1.90 (m, 3H), 1.79-1.72 (m, 2H), 1.65-1.50 (m, 4H).

### Example 72. Synthesis of N-(2-chlorophenyl)-4-((2-((4-((4-(1-(4-(4-(2,6-dioxopiperidin-3-yl)phenyl)piperazin-1-yl)butan-2-yl)piperidin-1-yl)carbamoyl)phenyl)amino)-5-fluoropyrimidin-4-yl) amino)benzamide (Compound 72)

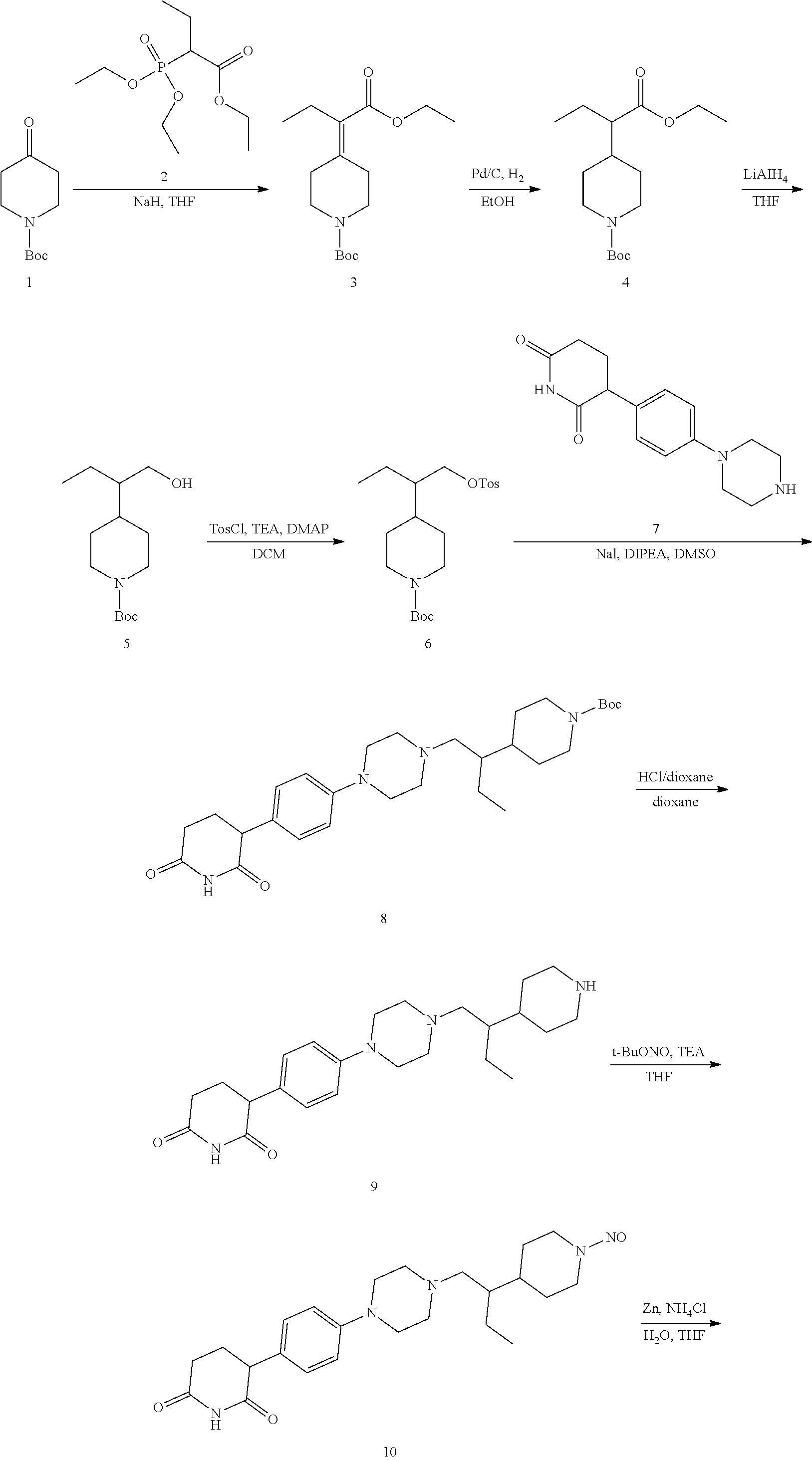

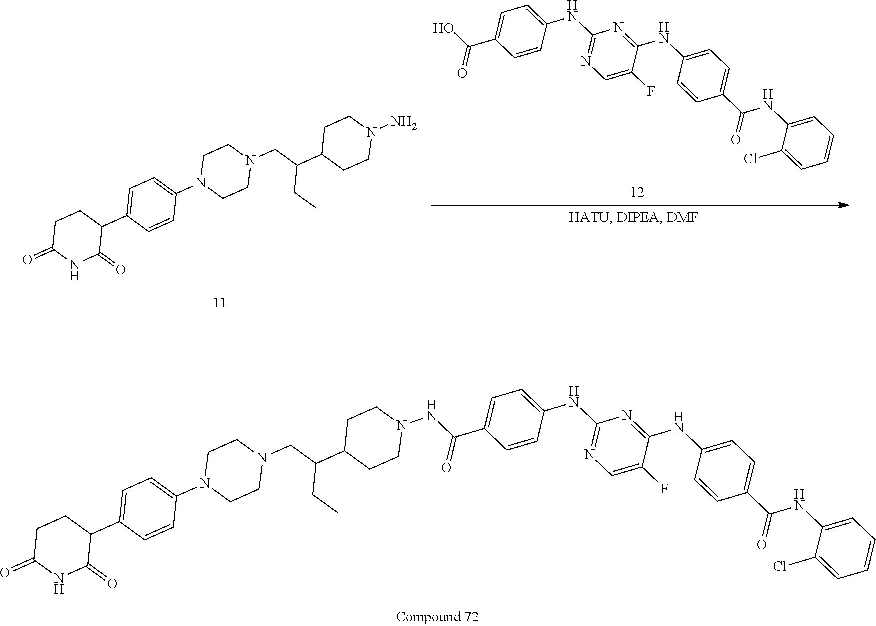 [SMILES: CCC(CN1CCN(c2ccc(C3CCC(=O)NC3=O)cc2)CC1)C1CCN(N)CC1 ; O=C(O)c1ccc(Nc2ncc(F)c(Nc3ccc(C(=O)Nc4ccccc4Cl)cc3)n2)cc1 ; CCC(CN1CCN(c2ccc(C3CCC(=O)NC3=O)cc2)CC1)C1CCN(NC(=O)c2ccc(Nc3ncc(F)c(Nc4ccc(C(=O)Nc5ccccc5Cl)cc4)n3)cc2)CC1]

### Step 1. Synthesis of tert-butyl 4-(1-ethoxy-1-oxobutan-2-ylidene) piperidine-1-carboxylate (3)

To a solution of ethyl 2-(diethoxyphosphoryl) butanoate (2.4 g, 9.51 mmol, 2.26 mL) in THF (20 mL) was added NaH (380.55 mg, 9.51 mmol, 60% purity) slowly at 0° C. for 0.5 hr under N₂ atmosphere. Then a solution of tert-butyl 4-oxopiperidine-1-carboxylate (1.52 g, 7.61 mmol) in THF (10 mL) was added in the reaction mixture and stirred at 25° C. for 1 hr under N₂ atmosphere. LCMS showed tert-butyl 4-oxopiperidine-1-carboxylate was consumed completely and desired mass was detected. The reaction mixture was poured into saturated NH₄Cl solution (50 mL) and extracted with EtOAc (20 mL×2). The combined organic layer was dried over Na₂SO₄, filtered and concentrated under reduced pressure to give a residue, which was purified by flash silica gel chromatography (ISCO®; 40 g SepaFlash® Silica Flash Column, Eluent of 0-10% EtOAc/Petroleum ether, gradient @ 80 mL/min) to afford tert-butyl 4-(1-ethoxy-1-oxobutan-2-ylidene)piperidine-1-carboxylate (1.05 g, 3.53 mmol, 37.11% yield) as a colorless oil. MS(M-Boc+H)+=198.3.

### Step 2. Synthesis of tert-butyl 4-(1-ethoxy-1-oxobutan-2-yl)piperidine-1-carboxylate (4)

To a solution of tert-butyl 4-(1-ethoxy-1-oxobutan-2-ylidene)piperidine-1-carboxylate (2.21 g, 7.43 mmol) in EtOH (22 mL) was added Pd/C (800 mg, 10% purity) under N₂. The suspension was degassed under vacuum and purged with H₂ several times. The mixture was stirred under H₂ (45 psi) at 25° C. for 16 hours. LCMS showed tert-butyl 4-(1-ethoxy-1-oxobutan-2-ylidene)piperidine-1-carboxylate was consumed completely and desired mass was detected. The mixture was filtered and the filter cake was washed with EtOH (150 mL) and THF (150 mL). The filtrate was concentrated to afford tert-butyl 4-(1-ethoxy-1-oxobutan-2-yl)piperidine-1-carboxylate (2.18 g, crude) as a colorless oil. MS(M−Boc+H)⁺=200.3.

### Step 3. Synthesis of tert-butyl 4-(1-hydroxybutan-2-yl)piperidine-1-carboxylate (5)

To a solution of tert-butyl 4-(1-ethoxy-1-oxobutan-2-yl)piperidine-1-carboxylate (2 g, 6.68 mmol) in THF (40 mL) was added LiAlH₄ (2.5 M, 2.67 mL) drop-wise at 0° C. under N₂ atmosphere. The mixture was stirred at 0° C. for 1 hr under N₂ atmosphere. LCMS showed tert-butyl 4-(1-ethoxy-1-oxobutan-2-yl)piperidine-1-carboxylate was consumed completely and desired mass was detected. The reaction mixture was quenched by addition H₂O (270 μL), 15% NaOH aq. (270 μL) and H₂O (810 μL), then dried over MgSO₄, filtered and concentrated under reduced pressure to afford tert-butyl 4-(1-hydroxybutan-2-yl)piperidine-1-carboxylate (1.69 g, crude) as a colorless oil. MS(M−isobutylene+H)⁺=202.1.

### Step 4. Synthesis of tert-butyl 4-(1-(tosyloxy)butan-2-yl)piperidine-1-carboxylate (6)

To a solution of tert-butyl 4-(1-hydroxybutan-2-yl)piperidine-1-carboxylate (1.64 g, 6.37 mmol) in DCM (17 mL) were added TosCl (1.82 g, 9.56 mmol), DMAP (77.85 mg, 637.22 μmol) and TEA (1.93 g, 19.12 mmol, 2.66 mL). The mixture was stirred at 25° C. for 16 hr. LCMS showed tert-butyl 4-(1-hydroxybutan-2-yl)piperidine-1-carboxylate was consumed completely and desired mass was detected. The reaction mixture was diluted with H₂O (50 mL) and extracted with EtOAc (20 mL×3). The combined organic layers were dried over Na₂ SO₄, filtered and concentrated under reduced pressure to give a residue, which was purified by flash silica gel chromatography (ISCO®; 20 g SepaFlash® Silica Flash Column, Eluent of 0-16% EtOAc/Petroleum ether, gradient @ 100 mL/min) to afford tert-butyl 4-(1-(tosyloxy)butan-2-yl)piperidine-1-carboxylate (2.26 g, 5.49 mmol, 86.18% yield) as a yellow oil. MS(M+H)⁺=412.3.

### Step 5. Synthesis of tert-butyl 4-(1-(4-(4-(2,6-dioxopiperidin-3-yl)phenyl)piperazin-1-yl)butan-2-yl)piperidine-1-carboxylate (8)

To a solution of tert-butyl 4-(1-(tosyloxy)butan-2-yl)piperidine-1-carboxylate (1.97 g, 4.79 mmol) and 3-(4-(piperazin-1-yl)phenyl)piperidine-2,6-dione (830 mg, 2.68 mmol, HCl) in DMSO (20 mL) were added NaI (71.86 mg, 479.42 μmol) and DIPEA (924.4 mg, 7.15 mmol, 1.25 mL). The mixture was stirred at 80° C. for 16 hr. LC-MS showed tert-butyl 4-(1-(tosyloxy)butan-2-yl)piperidine-1-carboxylate was consumed completely and desired mass was detected. The reaction mixture was diluted with H₂O (100 mL) and extracted with EtOAc (30 mL×3). The combined organic layers were washed with brine (40 mL), dried over Na₂SO₄, filtered and concentrated under reduced pressure to give a residue, which was purified by flash silica gel chromatography (ISCO®; 40 g SepaFlash® Silica Flash Column, Eluent of 0~50% EtOAc/Petroleum ether, gradient @ 80 mL/min) to afford tert-butyl 4-(1-(4-(4-(2,6-dioxopiperidin-3-yl)phenyl)piperazin-1-yl)butan-2-yl)piperidine-1-carboxylate (330 mg, 643.67 μmol, 24.02% yield) as a yellow solid. MS(M+H)⁺=513.4.

### Step 6. Synthesis of 3-(4-(4-(2-(piperidin-4-yl)butyl)piperazin-1-yl)phenyl)piperidine-2,6-dione (9)

To a solution of tert-butyl 4-(1-(4-(4-(2,6-dioxopiperidin-3-yl)phenyl)piperazin-1-yl)butan-2-yl)piperidine-1-carboxylate (370 mg, 721.69 μmol) in dioxane (4 mL) was added HCl/dioxane (4 M, 8 mL). The mixture was stirred at 25° C. for 1 hr. LCMS showed tert-butyl 4-(1-(4-(4-(2,6-dioxopiperidin-3-yl)phenyl)piperazin-1-yl)butan-2-yl)piperidine-1-carboxylate was consumed completely and desired mass was detected. The reaction mixture was concentrated under reduced pressure to afford 3-(4-(4-(2-(piperidin-4-yl)butyl)piperazin-1-yl)phenyl)piperidine-2,6-dione (320 mg, crude, HCl) as a yellow solid. MS(M+H)⁺=413.3.

### Step 7. Synthesis of 3-(4-(4-(2-(1-nitrosopiperidin-4-yl)butyl)piperazin-1-yl)phenyl)piperidine-2,6-dione (10)

To a solution of 3-(4-(4-(2-(piperidin-4-yl)butyl)piperazin-1-yl)phenyl)piperidine-2,6-dione (160 mg, 356.33 mol, HCl) in THF (1.6 mL) was added TEA (117.73 mg, 1.16 mmol, 161.94 μL) at 0° C., followed by the addition of t-BuONO (119.97 mg, 1.16 mmol, 138.37 μL) drop-wise. The resulting mixture was stirred at 60° C. for 16 hr. LCMS showed 40% of 3-(4-(4-(2-(piperidin-4-yl)butyl)piperazin-1-yl)phenyl)piperidine-2,6-dione remained. Another portion of t-BuONO (119.97 mg, 1.16 mmol, 138.37 μL) was added and the mixture was stirred at 60° C. for 2 hr. LCMS showed 3-(4-(4-(2-(piperidin-4-yl)butyl)piperazin-1-yl)phenyl)piperidine-2,6-dione was consumed completely and desired mass was detected. The reaction mixture was diluted with H₂O (20 mL) and extracted with EtOAc (10 mL×2). The combined organic layers were dried over Na₂SO₄, filtered and concentrated under reduced pressure to give a residue. The residue was purified by flash silica gel chromatography (ISCO®; 12 SepaFlash® Silica Flash Column, Eluent of 0-70% EtOAc/Petroleum ether, gradient @ 50 mL/min) to afford 3-(4-(4-(2-(1-nitrosopiperidin-4-yl)butyl)piperazin-1-yl)phenyl)piperidine-2,6-dione (43 mg, 97.38 μmol, 27.33% yield) as a yellow solid. MS(M+H)⁺=442.4.

### Step 8. Synthesis of 3-(4-(4-(2-(1-aminopiperidin-4-yl)butyl)piperazin-1-yl)phenyl)piperidine-2,6-dione (11)

To a solution of 3-(4-(4-(2-(1-nitrosopiperidin-4-yl)butyl)piperazin-1-yl)phenyl)piperidine-2,6-dione (43 mg, 97.38 μmol), 3-[4-[4-[2-(1-nitroso-4-piperidyl)butyl]piperazin-1-yl]phenyl]piperidine-2,6-dione (28.00 mg, 50.40 mol, TFA) and NH₄Cl (25 mg, 467.37 μmol) in THF (1.4 mL) and H₂O (0.7 mL) was added Zn (32 mg, 489.37 μmol) at 0° C. under N₂ atmosphere. The mixture was stirred at 20° C. for 3.5 hr under N₂ atmosphere. LCMS showed 15% of 3-(4-(4-(2-(1-nitrosopiperidin-4-yl)butyl)piperazin-1-yl)phenyl)piperidine-2,6-dione remained. The reaction mixture filtered and the filter cake was washed with THF (40 mL). The filtrate was concentrated under reduced pressure to afford 3-(4-(4-(2-(1-aminopiperidin-4-yl)butyl)piperazin-1-yl)phenyl)piperidine-2,6-dione (67 mg, crude) as a white solid. MS(M+H)⁺=428.3.

### Step 9. Synthesis of N-(2-chlorophenyl)-4-((2-((4-((4-(1-(4-(4-(2,6-dioxopiperidin-3-yl)phenyl)piperazin-1-yl)butan-2-yl)piperidin-1-yl)carbamoyl)phenyl)amino)-5-fluoropyrimidin-4-yl) amino)benzamide (Compound 72)

To a solution of 4-((4-((4-((2-chlorophenyl)carbamoyl)phenyl)amino)-5-fluoropyrimidin-2-yl)amino)benzoic acid (59.90 mg, 125.36 μmol) in DMF (1 mL) were added HATU (89.37 mg, 235.04 μmol) and DIPEA (101.26 mg, 783.48 μmol, 136.47 μL). After stirring at 25° C. for 0.5 hr, 3-(4-(4-(2-(1-aminopiperidin-4-yl)butyl)piperazin-1-yl)phenyl)piperidine-2,6-dione (67 mg, 156.70 μmol) was added and the resulting mixture and stirred at 25° C. for 16 hr. LCMS showed 3-(4-(4-(2-(1-aminopiperidin-4-yl)butyl)piperazin-1-yl)phenyl)piperidine-2,6-dione was consumed completely and desired mass was detected. The reaction mixture was H₂O (20 mL) and extracted with EtOAc (10 mL×2). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 0-100% EtOAc/Petroleum ether, gradient @ 50 mL/min). The impure product was purified by prep-HPLC (column: Waters Xbridge 150*25 mm*5 um; mobile phase: [water (NH₄ HCO₃)-ACN]; gradient: 41%-71% B over min) and lyophilized to afford N-(2-chlorophenyl)-4-((2-((4-((4-(1-(4-(4-(2,6-dioxopiperidin-3-yl)phenyl)piperazin-1-yl)butan-2-yl)piperidin-1-yl) carbamoyl)phenyl)amino)-5-fluoropyrimidin-4-yl)amino)benzamide (7.8 mg, 8.61 mol, 5.50% yield, 98% purity) as a white solid. MS(M+H)⁺=887.6.

¹H NMR (400 MHz, DMSO-d₆) δ=10.77 (s, 1H), 9.92 (s, 1H), 9.73 (s, 1H), 9.60 (s, 1H), 9.11 (s, 1H), 8.24 (d, J=3.5 Hz, 1H), 8.07-7.95 (m, 4H), 7.82-7.73 (m, 2H), 7.72-7.61 (m, 3H), 7.59-7.50 (m, 1H), 7.45-7.35 (m, 1H), 7.33-7.24 (m, 1H), 7.05 (br d, J=8.6 Hz, 2H), 6.89 (br d, J=8.8 Hz, 2H), 3.77-3.68 (m, 1H), 3.20-3.10 (m, 4H), 3.05-3.00 (m, 2H), 2.75-2.62 (m, 3H), 2.60-2.57 (m, 2H), 2.47-2.44 (m, 2H), 2.30-2.22 (m, 1H), 2.22-2.09 (m, 2H), 2.06-1.95 (m, 1H), 1.60-1.50 (m, 2H), 1.49-1.33 (m, 5H), 1.32-1.19 (m, 2H), 0.94-0.81 (m, 3H).

### Example 73. Synthesis of N-(2-chlorophenyl)-4-((2-((4-((1-(2-(4-(4-((2,6-dioxopiperidin-3-yl)amino)phenyl)piperazin-1-yl)ethyl)piperidin-4-yl)carbamoyl)phenyl)amino)-5-fluoropyrimidin-4-yl) amino)benzamide (Compound 73)

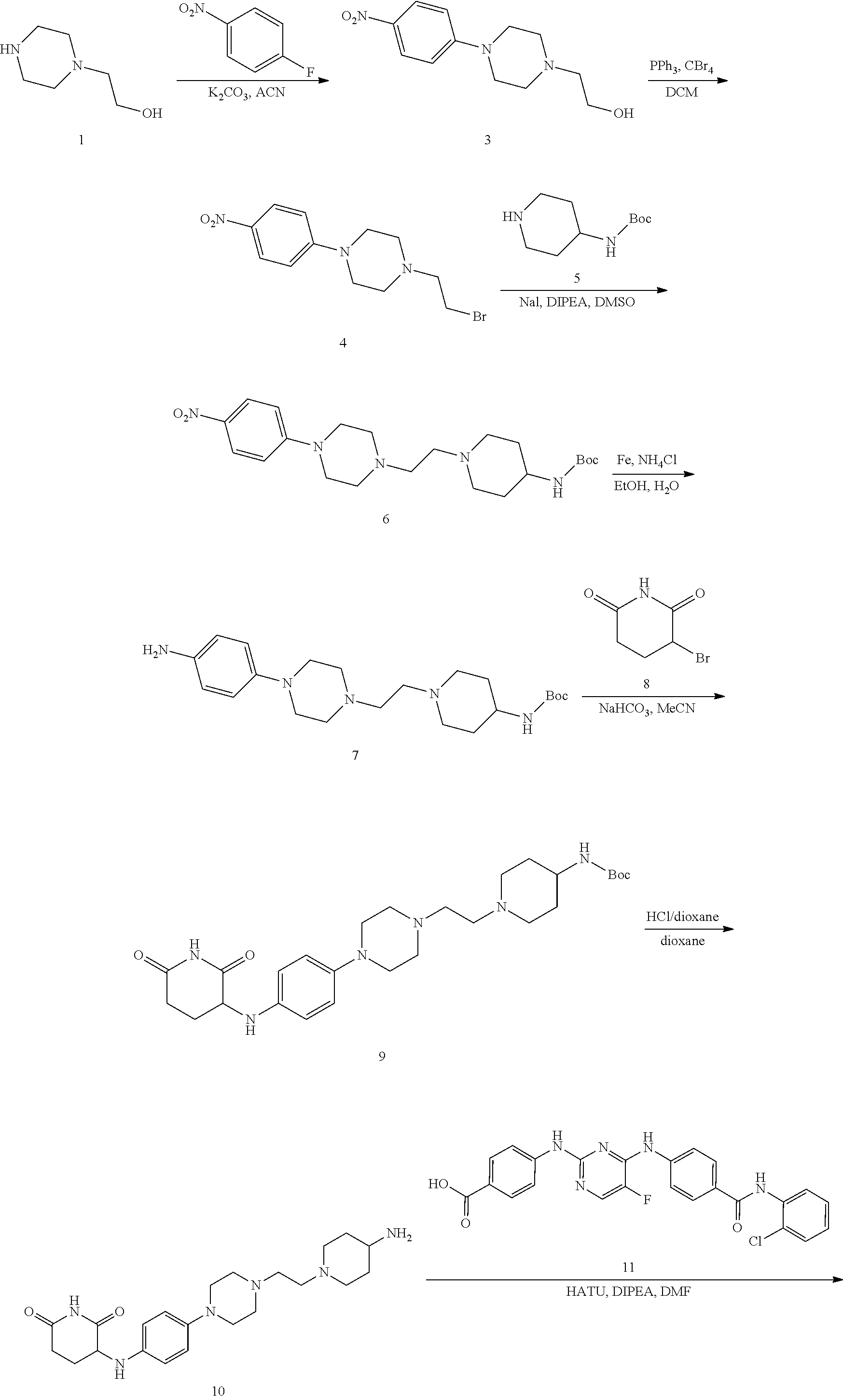

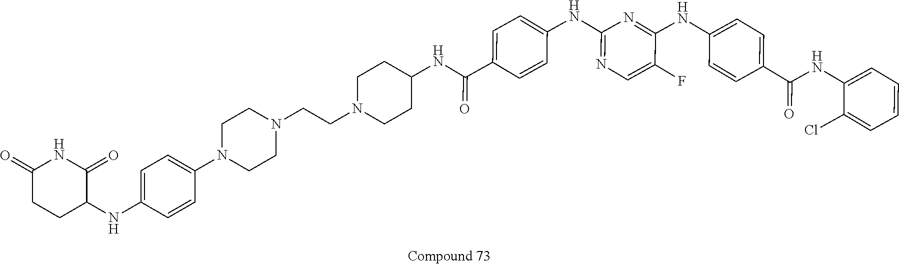 [SMILES: O=C1CCC(Nc2ccc(N3CCN(CCN4CCC(NC(=O)c5ccc(Nc6ncc(F)c(Nc7ccc(C(=O)Nc8ccccc8Cl)cc7)n6)cc5)CC4)CC3)cc2)C(=O)N1]

### Step 1. Synthesis of 2-(4-(4-nitrophenyl)piperazin-1-yl)ethan-1-ol (3)

To a solution of 2-(piperazin-1-yl)ethan-1-ol (5 g, 38.41 mmol, 4.71 mL) and 1-fluoro-4-nitrobenzene (5.42 g, 38.41 mmol, 4.07 mL) in ACN (100 mL) was added K₂CO₃ (15.92 g, 115.22 mmol). The mixture was stirred at 25° C. for 16 hr. LCMS showed 2-(piperazin-1-yl)ethan-1-ol was consumed completely and desired mass was detected. The reaction mixture was filtered and concentrated. The residue was diluted with H₂O (100 mL) and extracted with EtOAc (30 mL×3). The combined organic layers were dried over Na₂SO₄, filtered and concentrated under reduced pressure. The residue was purified by flash silica gel chromatography (ISCO®; 80 g SepaFlash® Silica Flash Column, Eluent of 0~20% MeOH/EtOAc @ 100 mL/min) to afford 2-(4-(4-nitrophenyl)piperazin-1-yl)ethan-1-ol (3.86 g, 15.36 mmol, 40.00% yield) as a yellow solid. MS(M+H)⁺=252.1.

### Step 2. Synthesis of 1-(2-bromoethyl)-4-(4-nitrophenyl)piperazine (4)

To a solution of 2-(4-(4-nitrophenyl)piperazin-1-yl)ethan-1-ol (1.5 g, 5.97 mmol) in DCM (30 mL) were added PPh₃ (2.04 g, 7.76 mmol) and CBr₄ (2.57 g, 7.76 mmol) at 0° C. The mixture was stirred at 25° C. for 16 hr under N₂ atmosphere. LCMS showed 2-(4-(4-nitrophenyl)piperazin-1-yl)ethan-1-ol was consumed completely and desired mass was detected. The mixture was filtered and concentrated to afford the residue, which was purified by flash silica gel chromatography (ISCO®; 20 g SepaFlash® Silica Flash Column, Eluent of 0-30% EtOAc/Petroleum ether, gradient @ 80 mL/min) to afford 1-(2-bromoethyl)-4-(4-nitrophenyl)piperazine (795 mg, 2.53 mmol, 42.39% yield) as a yellow solid. MS(M+H)⁺=314.1.

### Step 3. Synthesis of tert-butyl (1-(2-(4-(4-nitrophenyl)piperazin-1-yl)ethyl)piperidin-4-yl) carbamate (6)

To a solution of 1-(2-bromoethyl)-4-(4-nitrophenyl)piperazine (690.23 mg, 2.20 mmol) and tert-butyl piperidin-4-ylcarbamate (400 mg, 2.00 mmol) in DMSO (7 mL) were added NaI (59.87 mg, 399.45 μmol) and DIPEA (774.37 mg, 5.99 mmol, 1.04 mL). The mixture was stirred at 25° C. for 5 hr. LCMS showed 1-(2-bromoethyl)-4-(4-nitrophenyl)piperazine was consumed completely and desired mass was detected. The reaction was diluted with water (50 mL) and extracted with EtOAc (20 mL×2). The combined organic layers were washed with brine (20 mL), dried over Na₂SO₄, filtered and concentrated under reduced pressure to afford tert-butyl (1-(2-(4-(4-nitrophenyl)piperazin-1-yl)ethyl)piperidin-4-yl)carbamate (990 mg, crude) as a yellow solid. MS(M+H)⁺=434.3.

### Step 4. Synthesis of tert-butyl (1-(2-(4-(4-aminophenyl)piperazin-1-yl)ethyl)piperidin-4-yl) carbamate (7)

To a solution of tert-butyl (1-(2-(4-(4-nitrophenyl)piperazin-1-yl)ethyl)piperidin-4-yl)carbamate (490 mg, 1.13 mmol) in EtOH (10 mL) and H₂O (2.5 mL) were added Fe (315.59 mg, 5.65 mmol) and NH₄Cl (604.57 mg, 11.30 mmol). The mixture was stirred at 80° C. for 1 hr. TLC (DCM:MeOH=10:1) indicated tert-butyl (1-(2-(4-(4-nitrophenyl)piperazin-1-yl)ethyl)piperidin-4-yl)carbamate was consumed completely and one new spot formed. The reaction mixture was filtered, diluted with saturated NaHCO₃ solution (30 mL) and extracted with EtOAc (60 mL×2). The combined organic layers were dried over Na₂SO₄, filtered and concentrated under reduced pressure to afford tert-butyl (1-(2-(4-(4-aminophenyl)piperazin-1-yl)ethyl)piperidin-4-yl)carbamate (380 mg, crude) as a brown solid. MS(M+H)⁺=404.3.

### Step 5. Synthesis of tert-butyl (1-(2-(4-(4-((2,6-dioxopiperidin-3-yl)amino)phenyl)piperazin-1-yl)ethyl)piperidin-4-yl)carbamate (9)

To a solution of tert-butyl (1-(2-(4-(4-aminophenyl)piperazin-1-yl)ethyl)piperidin-4-yl)carbamate (380 mg, 941.62 μmol) and 3-bromopiperidine-2,6-dione (452.00 mg, 2.35 mmol) in MeCN (1.9 mL) was added NaHCO₃ (458.79 mg, 5.46 mmol). The mixture was stirred at 80° C. for 16 hr. LCMS showed tert-butyl (1-(2-(4-(4-aminophenyl)piperazin-1-yl)ethyl)piperidin-4-yl)carbamate was consumed completely and desired mass was detected. The reaction mixture was diluted with H₂O (30 mL) and extracted with EtOAc (10 mL×2). The combined organic layers were dried over Na₂SO₄, filtered and concentrated under reduced pressure. The residue was purified by flash silica gel chromatography (ISCO®; 20 g SepaFlash® Silica Flash Column, Eluent of 0-25% EtOAc/Petroleum ether, gradient @ 80 mL/min) to afford tert-butyl (1-(2-(4-(4-((2,6-dioxopiperidin-3-yl)amino)phenyl)piperazin-1-yl)ethyl)piperidin-4-yl)carbamate (340 mg, 660.63 μmol, 70.16% yield) as a black solid. MS(M+H)⁺=515.4.

### Step 6. Synthesis of 3-((4-(4-(2-(4-aminopiperidin-1-yl)ethyl)piperazin-1-yl)phenyl)amino)piperidine-2,6-dione (10)

To a solution of tert-butyl (1-(2-(4-(4-((2,6-dioxopiperidin-3-yl)amino)phenyl)piperazin-1-yl)ethyl)piperidin-4-yl)carbamate (150 mg, 291.46 μmol) in dioxane (1.5 mL) was added HCl/dioxane (4 M, 3 mL). The mixture was stirred at 25° C. for 1.5 hr. LC-MS showed tert-butyl (1-(2-(4-(4-((2,6-dioxopiperidin-3-yl)amino)phenyl)piperazin-1-yl)ethyl)piperidin-4-yl)carbamate was consumed completely and desired mass was detected. The reaction mixture was concentrated under reduced pressure to afford 3-((4-(4-(2-(4-aminopiperidin-1-yl)ethyl)piperazin-1-yl)phenyl)amino)piperidine-2,6-dione (130 mg, crude, HCl) as a white solid. MS(M+H)⁺=415.2.

### Step 7. Synthesis of N-(2-chlorophenyl)-4-((2-((4-((1-(2-(4-(4-((2,6-dioxopiperidin-3-yl)amino)phenyl)piperazin-1-yl)ethyl)piperidin-4-yl)carbamoyl)phenyl)amino)-5-fluoropyrimidin-4-yl) amino)benzamide (Compound 73)

To a solution of 4-((4-((4-((2-chlorophenyl)carbamoyl)phenyl)amino)-5-fluoropyrimidin-2-yl)amino)benzoic acid (137.75 mg, 288.25 μmol) in DMF (2 mL) were added HATU (164.40 mg, 432.37 μmol) and DIPEA (186.27 mg, 1.44 mmol, 251.04 μL). The mixture was stirred at 25° C. for 0.5 hr. Then the reaction mixture was added 3-((4-(4-(2-(4-aminopiperidin-1-yl)ethyl)piperazin-1-yl)phenyl)amino)piperidine-2,6-dione (130 mg, 288.25 mol, HCl) and stirred at 25° C. for 16 hr. LCMS showed 3-((4-(4-(2-(4-aminopiperidin-1-yl)ethyl)piperazin-1-yl)phenyl)amino)piperidine-2,6-dione was consumed completely and desired mass was detected. The reaction mixture was diluted with H₂O (30 mL) at 25° C., and then extracted with EtOAc (10 mL×2). The combined organic layers were washed with brine (20 mL), dried over Na₂SO₄, filtered and concentrated under reduced pressure. The residue was purified by flash silica gel chromatography (ISCO®; 12 g SepaFlash® Silica Flash Column, Eluent of 0-40% EtOAc/MeOH @ 50 mL/min). The impure product was purified by prep-HPLC (column: Phenomenex luna C18 150*25 mm*10 um; mobile phase: [water (TFA)-ACN]; gradient: 20%-50% B over 10 min). Then the impure product was purified by prep-HPLC (column: Waters Xbridge 150*25 mm 10 um; mobile phase: [water (NH₄HCO₃)-ACN]; gradient: 31%-61% B over 10 min). The eluent was lyophilized to afford N-(2-chlorophenyl)-4-((2-((4-((1-(2-(4-(4-((2,6-dioxopiperidin-3-yl)amino)phenyl)piperazin-1-yl)ethyl)piperidin-4-yl)carbamoyl)phenyl)amino)-5-fluoropyrimidin-4-yl)amino) benzamide (24.7 mg, 25.99 mol, 9.02% yield, 92% purity) as a off-white solid. MS(M+H)⁺=874.6.

¹H NMR (400 MHz, DMSO-d₆) δ=10.76 (s, 1H), 9.92 (s, 1H), 9.71 (s, 1H), 9.60 (s, 1H), 8.24 (d, J=3.5 Hz, 1H), 8.05-7.97 (m, 5H), 7.78-7.72 (m, 4H), 7.68-7.61 (m, 1H), 7.59-7.53 (m, 1H), 7.44-7.36 (m, 1H), 7.33-7.25 (m, 1H), 6.74 (d, J=8.8 Hz, 2H), 6.65-6.56 (m, 2H), 5.36 (d, J=7.2 Hz, 1H), 4.24-4.14 (m, 1H), 3.80-3.66 (m, 1H), 2.93-2.86 (m, 5H), 2.78-2.69 (m, 1H), 2.63-2.58 (m, 1H), 2.54-2.51 (m, 5H), 2.44-2.40 (m, 4H), 2.16-2.08 (m, 1H), 2.04-1.96 (m, 2H), 1.90-1.81 (m, 1H), 1.79-1.68 (m, 2H), 1.62-1.47 (m, 2H).

### Example 74. Synthesis of N-(2-chlorophenyl)-4-((2-((4-(((1r,4r)-4-(2-(4-(4-(2,6-dioxopiperidin-3-yl)-2-fluorophenyl)piperazin-1-yl)ethyl)cyclohexyl)carbamoyl)phenyl)amino)-5-fluoropyrimidin-4-yl)amino)benzamide (Compound 74)

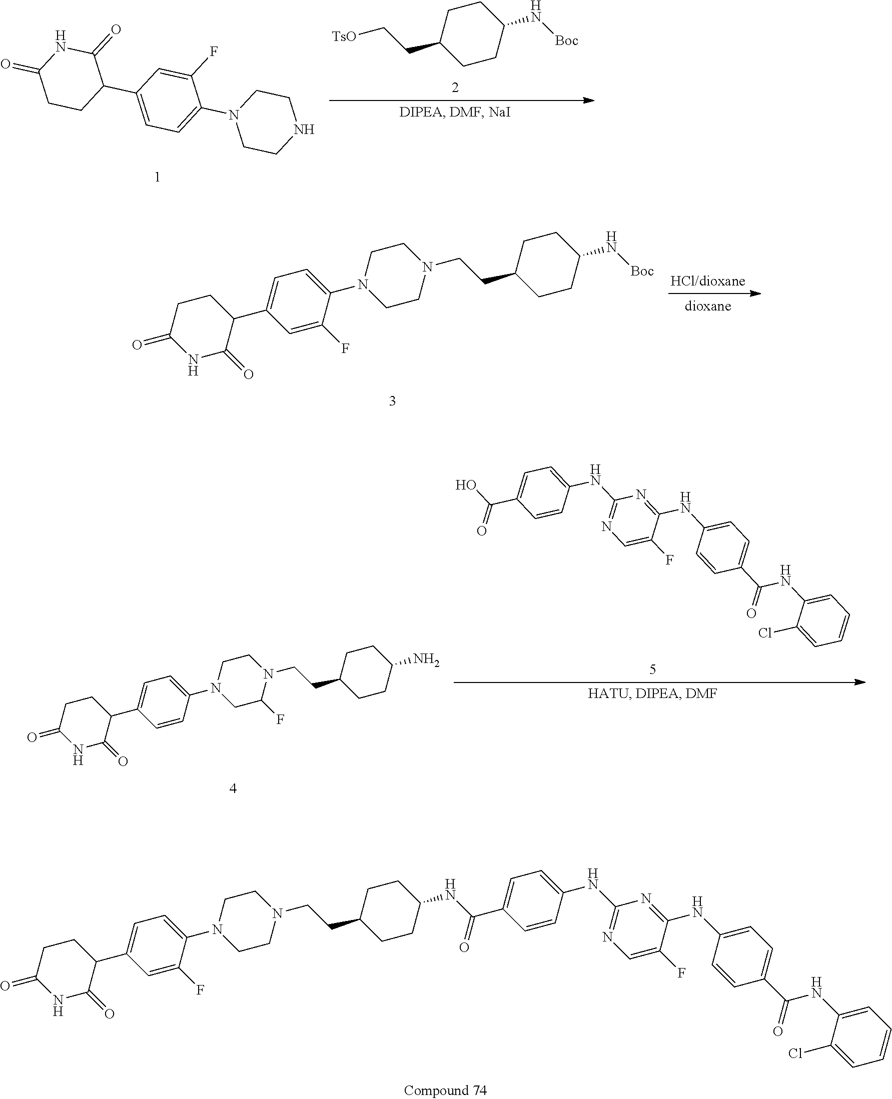 [SMILES: O=C1CCC(c2ccc(N3CCNCC3)c(F)c2)C(=O)N1 ; Cc1ccc(S(=O)(=O)OCC[C@H]2CC[C@H](NC(=O)OC(C)(C)C)CC2)cc1 ; CC(C)(C)OC(=O)N[C@H]1CC[C@H](CCN2CCN(c3ccc(C4CCC(=O)NC4=O)cc3F)CC2)CC1 ; N[C@H]1CC[C@H](CCN2CCN(c3ccc(C4CCC(=O)NC4=O)cc3)CC2F)CC1 ; O=C(O)c1ccc(Nc2ncc(F)c(Nc3ccc(C(=O)Nc4ccccc4Cl)cc3)n2)cc1 ; O=C1CCC(c2ccc(N3CCN(CC[C@H]4CC[C@H](NC(=O)c5ccc(Nc6ncc(F)c(Nc7ccc(C(=O)Nc8ccccc8Cl)cc7)n6)cc5)CC4)CC3)c(F)c2)C(=O)N1]

### Step 1. Synthesis of tert-butyl ((1r,4r)-4-(2-(4-(4-(2,6-dioxopiperidin-3-yl)-2-fluorophenylpiperazin-1-yl)ethyl)cyclohexyl)carbamate (3)

To a solution of 3-(3-fluoro-4-(piperazin-1-yl)phenyl)piperidine-2,6-dione (300 mg, 740.11 mol, TFA) and 2-((1r,4r)-4-((tert-butoxycarbonyl)amino)cyclohexyl)ethyl 4-methylbenzenesulfonate (294.22 mg, 740.11 μmol) in DMSO (3 mL) were added NaI (22.19 mg, 148.02 μmol) and DIPEA (573.93 mg, 4.44 mmol, 773.49 μL). The mixture was stirred at 80° C. for 16 hr. LCMS showed 3-(3-fluoro-4-(piperazin-1-yl)phenyl)piperidine-2,6-dione was consumed completely and desired mass was detected. The reaction was diluted with water (20 mL) and extracted with EtOAc (10 mL×2). The combined organic layers were washed with brine (10 mL), dried over anhydrous Na₂SO₄, filtered and concentrated under reduced pressure. The residue was purified by flash silica gel chromatography (ISCO®; 20 g SepaFlash® Silica Flash Column, Eluent of 0~100% EtOAc/Petroleum ether, gradient @ 60 mL/min) to afford tert-butyl ((1r,4r)-4-(2-(4-(4-(2,6-dioxopiperidin-3-yl)-2-fluorophenyl)piperazin-1-yl)ethyl)cyclo hexyl)carbamate (148 mg, 286.46 mol, 38.71% yield) as a brown solid. MS(M+H)⁺=517.4.

### Step 2. Synthesis of 3-(4-(4-(2-((1r,4r)-4-aminocyclohexyl)ethyl)piperazin-1-yl)-3-fluorophenyl)piperidine-2,6-dione (4)

To a solution of tert-butyl ((1r,4r)-4-(2-(4-(4-(2,6-dioxopiperidin-3-yl)-2-fluorophenyl)piperazin-1-yl)ethyl)cyclo hexyl)carbamate (148 mg, 286.46 μmol) in dioxane (1.5 mL) was added HCl/dioxane (4 M, 3 mL). The mixture was stirred at 25° C. for 1.5 hr. LCMS showed tert-butyl ((1r,4r)-4-(2-(4-(4-(2,6-dioxopiperidin-3-yl)-2-fluorophenyl)piperazin-1-yl)ethyl)cyclo hexyl)carbamate was consumed completely and desired mass. The reaction mixture was concentrated under reduced pressure to afford 3-(4-(4-(2-((1r,4r)-4-aminocyclohexyl)ethyl)piperazin-1-yl)-3-fluorophenyl)piperidine-2,6-dione (129 mg, crude, HCl) as a white solid. MS(M+H)⁺=417.3.

### Step 3. Synthesis of N-(2-chlorophenyl)-4-((2-((4-(((1r,4r)-4-(2-(4-(4-(2,6-dioxopiperidin-3-yl)-2-fluorophenyl)piperazin-1-yl)ethyl)cyclohexyl)carbamoyl)phenyl)amino)-5-fluoropyrimidin-4-yl)amino)benzamide (Compound 74)

To a solution of 4-((4-((4-((2-chlorophenyl)carbamoyl)phenyl)amino)-5-fluoropyrimidin-2-yl)amino)benzoic acid (136.09 mg, 284.77 μmol) in DMF (2 mL) were added HATU (162.42 mg, 427.16 μmol) and DIPEA (184.02 mg, 1.42 mmol, 248.01 μL). After stirring at 25° C. for 0.5 hr, 3-(4-(4-(2-((1r,4r)-4-aminocyclohexyl)ethyl)piperazin-1-yl)-3-fluorophenyl)piperidine-2,6-dione (129 mg, 284.77 μmol, HCl) was added and the resulting mixture was stirred at 25° C. for 16 hr. LCMS showed 3-(4-(4-(2-((1r,4r)-4-aminocyclohexyl)ethyl)piperazin-1-yl)-3-fluorophenyl)piperidine-2,6-dione was consumed completely and desired mass was detected. The reaction mixture was diluted water (20 mL) and extracted with EtOAc (10 mL×3). The combined organic layers were washed with brine (10 mL), dried over Na₂SO₄, filtered and concentrated under reduced pressure. The residue was triturated with DCM:MeOH=10:1 at 25° C. for 60 min to afford N-(2-chlorophenyl)-4-((2-((4-(((1r,4r)-4-(2-(4-(4-(2,6-dioxopiperidin-3-yl)-2-fluorophenyl)piperazin-1-yl)ethyl)cyclohexyl)carbamoyl)phenyl)amino)-5-fluoropyrimidin-4-yl)amino)benzamide (35.1 mg, 36.45 μmol, 12.80% yield, 91% purity) as a white solid. MS(M+H)⁺=876.3.

¹H NMR (400 MHz, DMSO-d₆) δ=10.82 (s, 1H), 9.94 (s, 1H), 9.75 (s, 1H), 9.61 (s, 1H), 8.25 (d, J=3.4 Hz, 1H), 8.07-7.98 (m, 4H), 7.95 (br d, J=8.1 Hz, 1H), 7.80-7.75 (m, 4H), 7.65 (d, J=7.1 Hz, 1H), 7.59-7.54 (m, 1H), 7.42-7.37 (m, 1H), 7.32-7.27 (m, 1H), 7.03 (br d, J=13.4 Hz, 1H), 7.00-6.93 (m, 2H), 3.84-3.77 (m, 1H), 3.76-3.65 (m, 1H), 3.10-2.95 (m, 4H), 2.71-2.59 (m, 2H), 2.58-2.53 (m, 3H), 2.40-2.30 (m, 2H), 2.26-2.13 (m, 1H), 2.05-1.94 (m, 1H), 1.87-1.72 (m, 4H), 1.43-1.18 (m, 6H), 1.09-0.95 (m, 2H).

### Example 75. Synthesis of N-(2-chlorophenyl)-4-((2-((4-(((1r,4r)-4-((2-(4-(4-(2,6-dioxopiperidin-3-yl)phenyl)piperazin-1-yl)ethyl)(methyl)amino)cyclohexyl)carbamoyl)phenyl)amino)-5-fluoropyrimidin-4-yl)amino)benzamide (Compound 75)

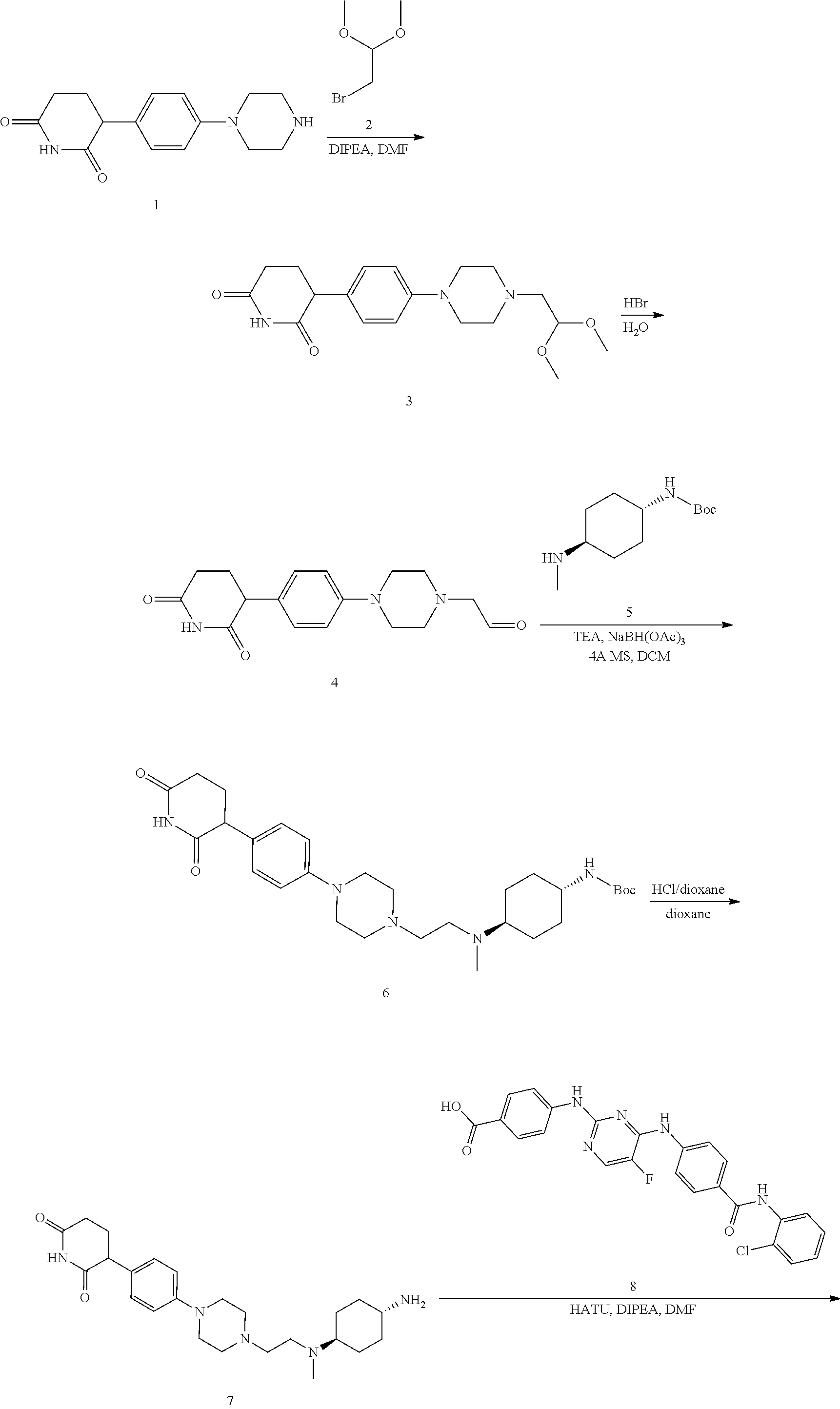

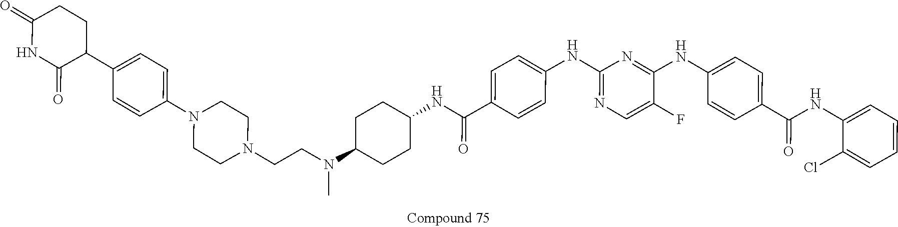 [SMILES: CN(CCN1CCN(c2ccc(C3CCC(=O)NC3=O)cc2)CC1)[C@H]1CC[C@H](NC(=O)c2ccc(Nc3ncc(F)c(Nc4ccc(C(=O)Nc5ccccc5Cl)cc4)n3)cc2)CC1]

### Step 1. Synthesis of 3-(4-(4-(2,2-dimethoxyethyl)piperazin-1-yl)phenyl)piperidine-2,6-dione (3)

To a solution of 3-(4-(piperazin-1-yl)phenyl)piperidine-2,6-dione (1 g, 3.23 mmol, HCl salt) and 2-bromo-1,1-dimethoxyethane (709.26 mg, 4.20 mmol, 492.54 μL) in DMF (10 mL) was added DIPEA (2.09 g, 16.14 mmol, 2.81 mL). The mixture was stirred at 100° C. for 16 hr. LCMS showed 3-(4-(piperazin-1-yl)phenyl)piperidine-2,6-dione was consumed completely and one main peak with desired mass was detected. The mixture was diluted with H₂O (30 mL) and saturated Na₂CO₃ solution (30 mL). The resulting mixture was extracted with EtOAc (20 mL×5). The combined organic layers were washed with brine (30 mL×2), dried over Na₂SO₄, filtered and concentrated in vacuum. The residue was purified by flash silica gel chromatography (ISCO®; 20 g SepaFlash® Silica Flash Column, Eluent of 90-100% EtOAc/Petroleum ether gradient @ 80 mL/min) to afford 3-(4-(4-(2,2-dimethoxyethyl)piperazin-1-yl)phenyl)piperidine-2,6-dione (500 mg, 1.38 mmol, 42.86% yield) as a light yellow solid. MS(M+H)⁺=362.2.

### Step 2. Synthesis of 2-(4-(4-(2,6-dioxopiperidin-3-yl)phenyl)piperazin-1-yl)acetaldehyde (4)

To a solution of 3-(4-(4-(2,2-dimethoxyethyl)piperazin-1-yl)phenyl)piperidine-2,6-dione (500 mg, 1.38 mmol) in H₂O (2.5 mL) was added HBr (7.45 g, 36.83 mmol, 5 mL, 40% purity). The mixture was stirred at 40° C. for 16 hr. LCMS showed 3-(4-(4-(2,2-dimethoxyethyl)piperazin-1-yl)phenyl)piperidine-2,6-dione was consumed completely and one main peak with desired mass was detected. The reaction mixture was adjusted pH=7 by NaHCO₃ solution at 0° C. The resulting mixture was extracted with EtOAc (20 mL×5). The combined organic layers were dried over Na₂ SO₄, filtered and concentrated under reduced pressure to afford 2-(4-(4-(2,6-dioxopiperidin-3-yl)phenyl)piperazin-1-yl)acetaldehyde (215 mg, 681.75 mol, 49.28% yield) as a white solid, which was used directly. MS(M+H₂O+H)⁺=334.1.

### Step 3. Synthesis of tert-butyl ((1r,4r)-4-((2-(4-(4-(2,6-dioxopiperidin-3-yl)phenyl)piperazin-1-yl)ethyl)(methyl)amino)cyclohexyl)carbamate (6)

To a solution of 2-(4-(4-(2,6-dioxopiperidin-3-yl)phenyl)piperazin-1-yl)acetaldehyde (215 mg, 681.75 μmol) and tert-butyl ((1r,4r)-4-(methylamino)cyclohexyl)carbamate (202.36 mg, 886.27 μmol) in DCM (5 mL) were added TEA (68.99 mg, 681.75 mol, 94.89 μL) and 4 Å MS (50 mg). After stirring at 25° C. for 30 min, NaBH(OAc)₃ (433.47 mg, 2.05 mmol) was added and the resulting mixture was stirred at 25° C. for 16 hr. LCMS showed 2-(4-(4-(2,6-dioxopiperidin-3-yl)phenyl)piperazin-1-yl)acetaldehyde was consumed completely and one main peak with desired mass was detected. The residue was diluted with NaHCO₃ solution (30 mL) and extracted with DCM (20 mL×3). The combined organic layers were washed with water (20 mL), dried over Na₂SO₄, filtered and concentrated under reduced pressure. The residue was purified by flash silica gel chromatography (ISCO®; 20 g SepaFlash® Silica Flash Column, Eluent of 50-60% MeOH/EtOAc ether gradient @ 80 mL/min) to afford tert-butyl ((1r,4r)-4-((2-(4-(4-(2,6-dioxopiperidin-3-yl)phenyl)piperazin-1-yl)ethyl)(methyl)amino)cyclohexyl)carbamate (230 mg, 435.86 μmol, 63.93% yield) as a white solid. MS(M+H)⁺=528.3.

### Step 4. Synthesis of 3-(4-(4-(2-(((1r,4r)-4-aminocyclohexyl)(methyl)amino)ethyl)piperazin-1-yl)phenyl)piperidine-2,6-dione (7)

To a solution of tert-butyl ((1r,4r)-4-((2-(4-(4-(2,6-dioxopiperidin-3-yl)phenyl)piperazin-1-yl)ethyl)(methyl)amino)cyclohexyl)carbamate (230 mg, 435.86 μmol) in dioxane (2 mL) was added HCl/dioxane (4 M, 2 mL). The mixture was stirred at 25° C. for 1 hr. LCMS showed one main peak with desired mass was detected. The reaction mixture was concentrated under reduced pressure to afford 3-(4-(4-(2-(((1r,4r)-4-aminocyclohexyl)(methyl)amino)ethyl)piperazin-1-yl)phenyl)piperidine-2,6-dione (210 mg, crude, HCl salt) was obtained as a white solid. MS(M+H)⁺=428.3.

### Step 5. Synthesis of N-(2-chlorophenyl)-4-((2-((4-(((1r,4r)-4-((2-(4-(4-(2,6-dioxopiperidin-3-yl)phenyl)piperazin-1-yl)ethyl)(methyl)amino)cyclohexyl)carbamoyl)phenyl)amino)-5-fluoropyrimidin-4-yl)amino)benzamide (Compound 75)

To a solution of 4-((4-((4-((2-chlorophenyl)carbamoyl)phenyl)amino)-5-fluoropyrimidin-2-yl)amino)benzoic acid (180 mg, 376.67 μmol) in DMF (2 mL) were added HATU (214.83 mg, 565.00 μmol) and DIPEA (243.40 mg, 1.88 mmol, 328.04 μL), followed by the addition of 3-(4-(4-(2-(((1r,4r)-4-aminocyclohexyl)(methyl)amino)ethyl)piperazin-1-yl)phenyl)piperidine-2,6-dione (209.75 mg, 452.00 mol, HCl salt). The resulting mixture was stirred at 25° C. for 2 hr. LCMS showed one main peak with desired mass was detected. The residue was diluted with water (20 mL) and extracted with EtOAc (10 mL×3). The combined organic layers were washed with brine (10 mL×3), dried over Na₂SO₄, filtered and concentrated under reduced pressure. The residue was purified by prep-HPLC (column: Phenomenex luna C18 150×40 mm×15 μm; mobile phase: [water(TFA)-ACN]; gradient: 15%-45% B over 10 min) and lyophilized. The impure product was further purified by prep-HPLC (column: Waters Xbridge 150×25 mm×5 m; mobile phase: [water(NH₄HCO₃)-ACN]; gradient: 29%-59% B over min) and lyophilized to afford N-(2-chlorophenyl)-4-((2-((4-(((1r,4r)-4-((2-(4-(4-(2,6-dioxopiperidin-3-yl)phenyl)piperazin-1-yl)ethyl)(methyl)amino)cyclohexyl)carbamoyl)phenyl)amino)-5-fluoropyrimidin-4-yl)amino)benzamide (39.4 mg, 43.47 mol, 11.54% yield, 97.9% purity) as a white solid. MS(M+H)⁺=887.3.

¹H NMR (400 MHz, DMSO-d₆) δ=10.78 (s, 1H), 9.94 (s, 1H), 9.75 (s, 1H), 9.61 (s, 1H), 8.25 (d, J=3.4 Hz, 1H), 8.05-7.99 (m, 4H), 7.96 (br d, J=7.7 Hz, 1H), 7.80-7.73 (m, 4H), 7.68-7.62 (m, 1H), 7.59-7.54 (m, 1H), 7.43-7.36 (m, 1H), 7.33-7.26 (m, 1H), 7.04 (d, J=8.7 Hz, 2H), 6.88 (d, J=8.7 Hz, 2H), 3.75-3.65 (m, 2H), 3.15-3.05 (m, 4H), 2.65-2.57 (m, 2H), 2.56-2.52 (m, 6H), 2.45-2.37 (m, 3H), 2.20 (s, 3H), 2.15-2.07 (m, 1H), 2.04-1.96 (m, 1H), 1.91-1.82 (m, 2H), 1.78-1.69 (m, 2H), 1.38-1.26 (m, 4H).

### Example 76. Synthesis of N-(2-chlorophenyl)-4-((2-((4-(((1r,3r)-3-(((1-(4-(2,6-dioxopiperidin-3-yl)-2-fluorophenyl)piperidin-4-yl)methyl)(methyl)amino)cyclobutyl)carbamoyl)phenyl)amino)-5-fluoropyrimidin-4-yl)amino)benzamide (Compound 76)

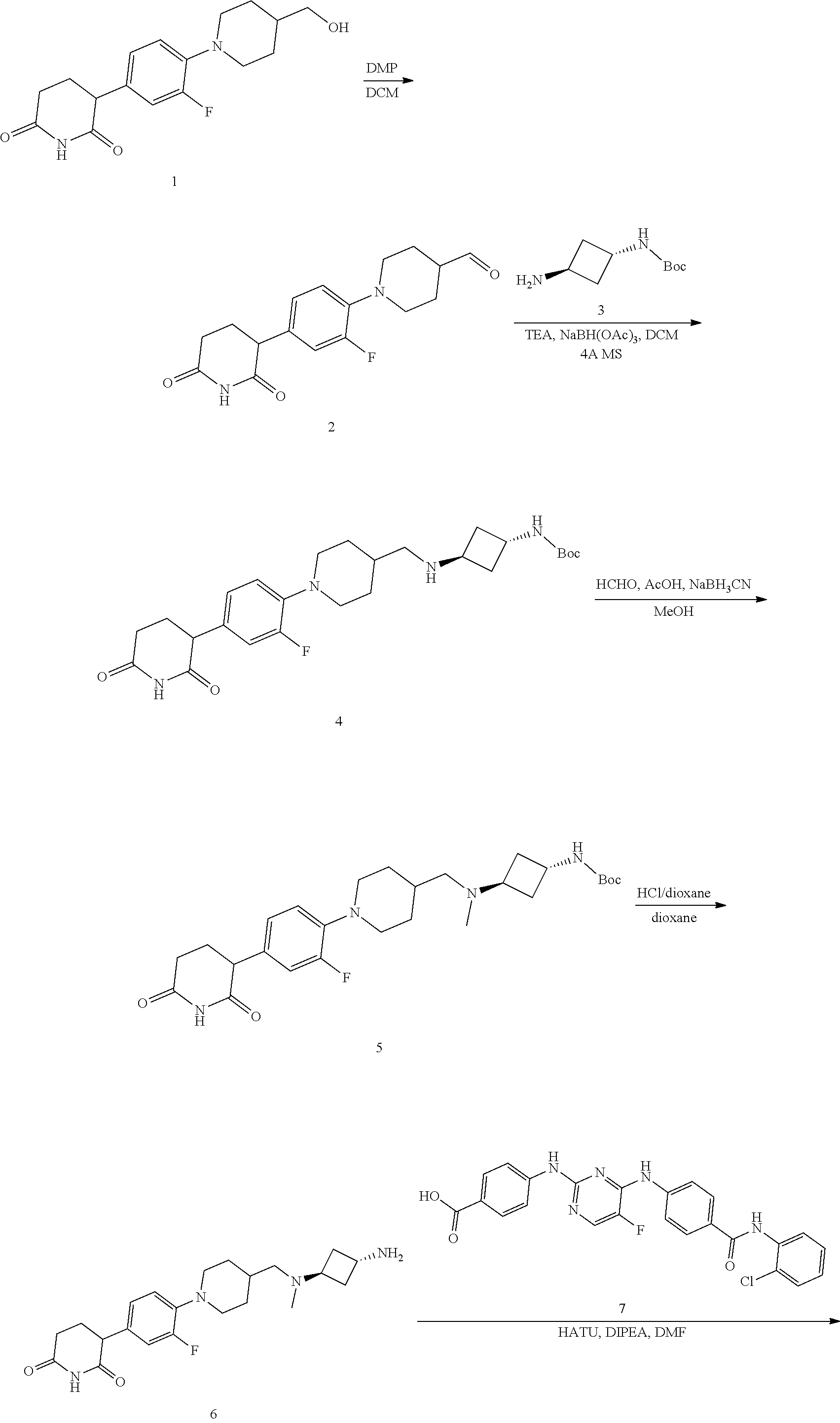

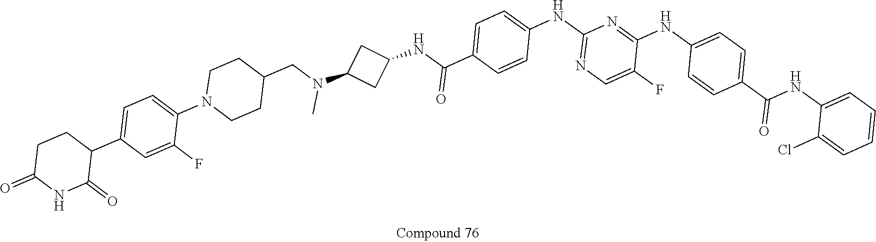 [SMILES: CN(CC1CCN(c2ccc(C3CCC(=O)NC3=O)cc2F)CC1)[C@H]1C[C@H](NC(=O)c2ccc(Nc3ncc(F)c(Nc4ccc(C(=O)Nc5ccccc5Cl)cc4)n3)cc2)C1]

### Step 1. Synthesis of 1-(4-(2,6-dioxopiperidin-3-yl)-2-fluorophenyl)piperidine-4-carbaldehyde (2)

To a solution of 3-(3-fluoro-4-(4-(hydroxymethyl)piperidin-1-yl)phenyl)piperidine-2,6-dione (1 g, 3.12 mmol) in DCM (10 mL) was added DMP (1.46 g, 3.43 mmol). The mixture was stirred at 25° C. for 2 hr. TLC (petroleum ether:EtOAc=1:2) showed the starting material was consumed completely and one new spot formed. The reaction mixture was filtered and concentrated under reduced pressure to give a residue to afford 1-(4-(2,6-dioxopiperidin-3-yl)-2-fluorophenyl)piperidine-4-carbaldehyde (1.6 g, 2.51 mmol, 80.51% yield, 50% purity) as a white solid, which was used directly. MS(M+H)⁺=319.4.

¹H NMR (400 MHz, CDCl₃) δ=9.73-9.68 (m, 1H), 8.61 (br s, 1H), 6.99-6.86 (m, 3H), 3.72 (dd, J=5.2, 9.8 Hz, 1H), 3.44-3.35 (m, 2H), 2.87-2.78 (m, 2H), 2.78-2.70 (m, 1H), 2.69-2.60 (m, 1H), 2.45-2.35 (m, 1H), 2.32-2.27 (m, 1H), 2.24-2.17 (m, 1H), 2.07-2.00 (m, 2H), 1.92-1.79 (m, 2H).

### Step 2. Synthesis of tert-butyl ((1r,3r)-3-(((1-(4-(2,6-dioxopiperidin-3-yl-2-fluorophenyl)piperidin-4-yl)methyl)amino)cyclobutyl)carbamate (4)

To a solution of 1-(4-(2,6-dioxopiperidin-3-yl)-2-fluorophenyl)piperidine-4-carbaldehyde (800 mg, 1.26 mmol, 50% purity) and tert-butyl ((1r,3r)-3-aminocyclobutyl)carbamate (234.03 mg, 1.26 mmol) in DCM (10 mL) was added TEA (127.15 mg, 1.26 mmol, 174.89 μL) and 4 Å MS (100 mg). After stirring at 25° C. for 30 min, NaBH(OAc)₃ (798.91 mg, 3.77 mmol) was added and the resulting mixture was stirred at 25° C. for 5 hr. LCMS showed 1-(4-(2,6-dioxopiperidin-3-yl)-2-fluorophenyl)piperidine-4-carbaldehyde was consumed completely and one main peak with desired mass was detected. The residue was diluted with NaHCO₃ solution (50 mL) and extracted with DCM (30 mL×3). The combined organic layers were washed with water (30 mL), dried over Na₂SO₄, filtered and concentrated under reduced pressure. The residue was purified by flash silica gel chromatography (ISCO®; 40 g SepaFlash® Silica Flash Column, Eluent of 30-40% MeOH/EtOAc gradient @ 80 mL/min) to afford tert-butyl ((1r,3r)-3-(((1-(4-(2,6-dioxopiperidin-3-yl)-2-fluorophenyl)piperidin-4-yl)methyl)amino)cyclobutyl)carbamate (330 mg, 675.41 mol, 53.75% yield) as a light yellow solid. MS(M+H)⁺=489.1.

### Step 3. Synthesis of tert-butyl ((1r,3r)-3-(((1-(4-(2,6-dioxopiperidin-3-yl)-2-fluorophenyl)piperidin-4-yl)methyl)(methyl)amino)cyclobutyl)carbamate (5)

To a solution of tert-butyl ((1r,3r)-3-(((1-(4-(2,6-dioxopiperidin-3-yl)-2-fluorophenyl)piperidin-4-yl)methyl)amino)cyclobutyl)carbamate (330 mg, 675.41 μmol) in MeOH (5 mL) were added HCHO (54.82 mg, 675.41 μmol, 50.29 μL, 37% purity) and AcOH (4.06 mg, 67.54 mol, 3.87 μL). After stirring at 25° C. for 30 min, NaBH₃CN (127.33 mg, 2.03 mmol) was added to the reaction mixture and the resulting mixture was stirred at 25° C. for 16 hr. LCMS showed tert-butyl ((1r,3r)-3-(((1-(4-(2,6-dioxopiperidin-3-yl)-2-fluorophenyl)piperidin-4-yl)methyl)amino)cyclobutyl)carbamate was consumed completely and one main peak with desired mass was detected. The residue was diluted with NaHCO₃ solution (20 mL) and extracted with EtOAc (20 mL×3). The combined organic layers were washed with water (20 mL), dried over Na₂SO₄, filtered and concentrated under reduced pressure to afford tert-butyl ((1r,3r)-3-(((1-(4-(2,6-dioxopiperidin-3-yl)-2-fluorophenyl)piperidin-4-yl)methyl)(methyl)amino)cyclobutyl)carbamate (310 mg, 616.77 mol, 91.32% yield) as a light yellow solid. MS(M+H)⁺=503.4.

### Step 4. Synthesis of 3-(4-(4-((((1r,3r)-3-aminocyclobutyl)(methyl)amino)methyl)piperidin-1-yl)-3-fluorophenyl)piperidine-2,6-dione (6)

To a solution of tert-butyl ((1r,3r)-3-(((1-(4-(2,6-dioxopiperidin-3-yl)-2-fluorophenyl)piperidin-4-yl)methyl)(methyl)amino)cyclobutyl)carbamate (290 mg, 576.98 μmol) in dioxane (3 mL) was added HCl/dioxane (4 M, 3 mL). The mixture was stirred at 25° C. for 1 hr. LCMS showed one main peak with desired mass was detected. The reaction mixture was concentrated under reduced pressure to afford 3-(4-(4-((((1r,3r)-3-aminocyclobutyl)(methyl)amino)methyl)piperidin-1-yl)-3-fluorophenyl)piperidine-2,6-dione (250 mg, 569.52 μmol, 98.71% yield, HCl salt) as a white solid. MS(M+H)⁺=403.2.

### Step 5. Synthesis of N-(2-chlorophenyl)-4-((2-((4-(((1r,3r)-3-(((1-(4-(2,6-dioxopiperidin-3-yl)-2-fluorophenyl)piperidin-4-yl)methyl)(methyl)amino)cyclobutyl)carbamoyl)phenyl)amino)-5-fluoropyrimidin-4-yl)amino)benzamide (Compound 76)

To a solution of 4-((4-((4-((2-chlorophenyl)carbamoyl)phenyl)amino)-5-fluoropyrimidin-2-yl)amino)benzoic acid (200 mg, 418.52 μmol) in DMF (4 mL) was added HATU (238.70 mg, 627.78 μmol) and DIPEA (270.45 mg, 2.09 mmol, 364.48 μL) and 3-(4-(4-((((1r,3r)-3-aminocyclobutyl)(methyl)amino)methyl)piperidin-1-yl)-3-fluorophenyl)piperidine-2,6-dione (220.46 mg, 502.22 mol, HCl salt). The mixture was stirred at 25° C. for 2 hr. LCMS showed 3-(4-(4-((((1r,3r)-3-aminocyclobutyl)(methyl)amino)methyl)piperidin-1-yl)-3-fluorophenyl)piperidine-2,6-dione was consumed completely and one main peak with desired mass was detected. The reaction mixture was poured into water (20 mL) and filtered. The filter cake was purified by prep-HPLC (column: Phenomenex luna C18 150×40 mm×15 μm; mobile phase: [water(TFA)-ACN]; gradient: 20%-50% B over 10 min) and lyophilized to afford the impure product, which was further purified by prep-HPLC (column: Waters Xbridge C18 150×50 mm×10 μm; mobile phase: [water(NH₄HCO₃)-ACN]; gradient: 35%-65% B over 10 min) and lyophilized to afford N-(2-chlorophenyl)-4-((2-((4-(((1r,3r)-3-(((1-(4-(2,6-dioxopiperidin-3-yl)-2-fluorophenyl)piperidin-4-yl)methyl)(methyl)amino)cyclobutyl)carbamoyl)phenyl)amino)-5-fluoropyrimidin-4-yl)amino) benzamide (41.5 mg, 47.98 mol, 11.46% yield, 99.7% purity) as a white solid. MS(M+H)⁺=862.4.

¹H NMR (400 MHz, DMSO-d₆) δ=10.82 (s, 1H), 9.97 (s, 1H), 9.75 (s, 1H), 9.66-9.59 (m, 1H), 8.68-8.57 (m, 1H), 8.25 (d, J=3.5 Hz, 1H), 8.06-7.99 (m, 4H), 7.84-7.76 (m, 4H), 7.65 (dd, J=1.3, 8.0 Hz, 1H), 7.56 (dd, J=1.3, 8.1 Hz, 1H), 7.42-7.37 (m, 1H), 7.33-7.26 (m, 1H), 7.06-6.93 (m, 3H), 4.43-4.27 (m, 1H), 4.04-3.87 (m, 1H), 3.80 (dd, J=4.9, 11.6 Hz, 1H), 3.39-3.34 (m, 2H), 3.08-2.93 (m, 1H), 2.92-2.82 (m, 1H), 2.78-2.71 (m, 3H), 2.70-2.60 (m, 4H), 2.47-2.34 (m, 2H), 2.26-2.09 (m, 2H), 2.05-1.97 (m, 2H), 1.96-1.85 (m, 1H), 1.80-1.69 (m, 1H), 1.52-1.33 (m, 2H), 1.31-1.20 (m, 1H).

### Example 77. Synthesis of N-(2-chlorophenyl)-4-((2-((4-((2-((1-(4-(2,6-dioxopiperidin-3-yl)-2-fluorophenyl)piperidin-4-yl)methyl)-2-azaspiro[3.3]heptan-6-yl)carbamoyl)phenyl)amino)-5-fluoro pyrimidin-4-yl)amino)benzamide (Compound 77)

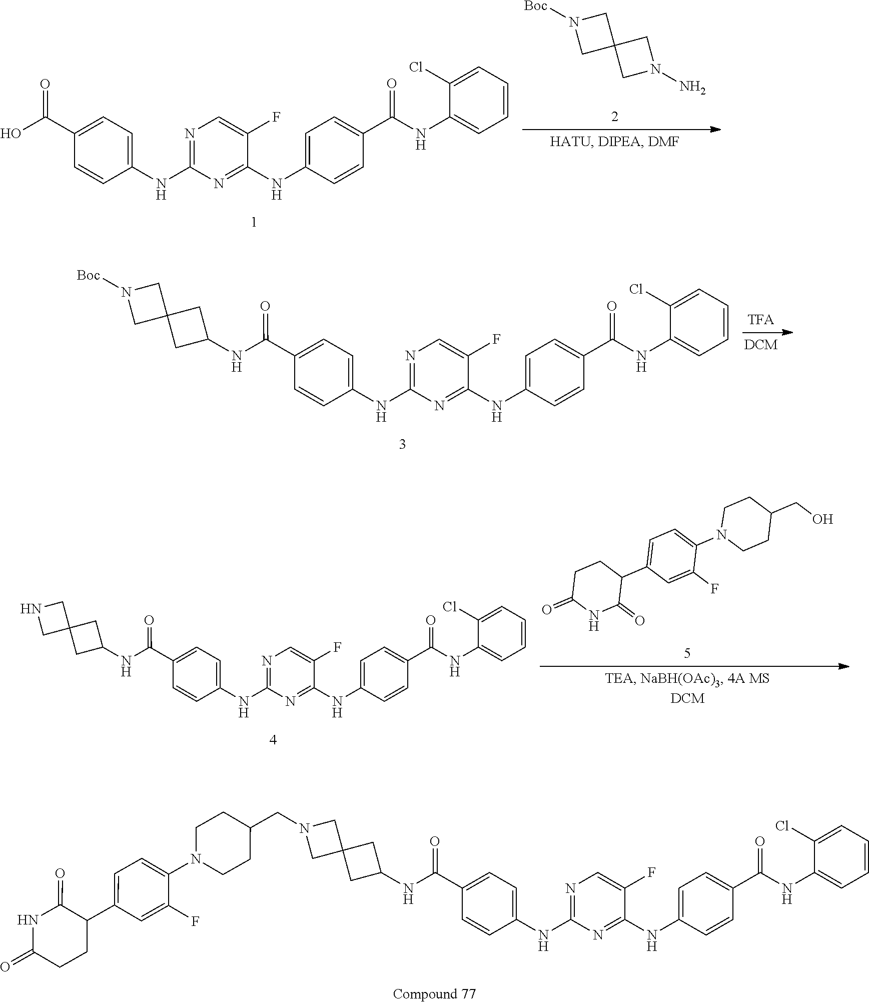

### Step 1. Synthesis of tert-butyl 6-(4-((4-((4-((2-chlorophenyl)carbamoyl)phenyl)amino)-5-fluoropyrimidin-2-yl)amino)benzamido)-2-azaspiro[3.3]heptane-2-carboxylate (3)

To a solution of 4-((4-((4-((2-chlorophenyl)carbamoyl)phenyl)amino)-5-fluoropyrimidin-2-yl)amino)benzoic acid (300 mg, 502.22 μmol) in DMF (5 mL) were added HATU (286.44 mg, 753.34 μmol) and DIPEA (324.54 mg, 2.51 mmol, 437.38 μL), followed by the addition of tert-butyl 6-amino-2-azaspiro[3.3]heptane-2-carboxylate (127.94 mg, 602.67 μmol). The resulting mixture was stirred at 25° C. for 2 hr. LCMS showed one main peak with desired mass. The residue was diluted with water (30 mL) and extracted with EtOAc (20 mL×3). The combined organic layers were washed with brine (10 mL×3), dried over Na₂SO₄, filtered and concentrated under reduced pressure. The crude product was triturated with EtOAc (20 mL) to afford tert-butyl 6-(4-((4-((4-((2-chlorophenyl)carbamoyl)phenyl)amino)-5-fluoropyrimidin-2-yl)amino)benzamido)-2-azaspiro[3.3]heptane-2-carboxylate (330 mg, 490.96 mol, 97.76% yield) as a yellow solid. MS(M+H)⁺=672.3.

### Step 2. Synthesis of 4-((2-((4-((2-azaspiro[3.3]heptan-6-yl)carbamoyl)phenyl)amino)-5-fluoropyrimidin-4-yl)amino)-N-(2-chlorophenyl)benzamide (4)

To a solution of tert-butyl 6-(4-((4-((4-((2-chlorophenyl)carbamoyl)phenyl)amino)-5-fluoropyrimidin-2-yl)amino)benzamido)-2-azaspiro[3.3]heptane-2-carboxylate (280 mg, 416.58 μmol) in DCM (6 mL) was added TFA (3.07 g, 26.93 mmol, 2.00 mL). The mixture was stirred at 25° C. for 1 hr. LCMS showed the starting material was consumed completely and one main peak with desired mass. The reaction mixture was concentrated under reduced pressure to afford 4-((2-((4-((2-azaspiro[3.3]heptan-6-yl)carbamoyl)phenyl)amino)-5-fluoropyrimidin-4-yl)amino)-N-(2-chlorophenyl)benzamide (300 mg, crude, TFA salt) as a light yellow oil. MS(M+H)⁺=572.2.

### Step 3. Synthesis of N-(2-chlorophenyl)-4-((2-((4-((2-((1-(4-(2,6-dioxopiperidin-3-yl)-2-fluorophenyl)piperidin-4-yl)methyl)-2-azaspiro[3.3]heptan-6-yl)carbamoyl)phenyl)amino)-5-fluoropyrimidin-4-yl)amino)benzamide (Compound 77)

To a solution of 1-(4-(2,6-dioxopiperidin-3-yl)-2-fluorophenyl)piperidine-4-carbaldehyde (180 mg, 282.71 μmol) and 4-((2-((4-((2-azaspiro[3.3]heptan-6-yl)carbamoyl)phenyl)amino)-5-fluoropyrimidin-4-yl)amino)-N-(2-chlorophenyl)benzamide (290.94 mg, 424.07 μmol, TFA salt) in DCM (5 mL) were added TEA (28.61 mg, 282.71 μmol, 39.35 μL) and 4 Å MS (50 mg). After stirring at 25° C. for 30 min, NaBH(OAc)₃ (179.76 mg, 848.14 μmol) was added and the resulting mixture was stirred at 25° C. for 16 hr. LCMS showed one main peak with desired mass. The reaction mixture was poured into NaHCO₃ solution (20 mL) and filtered. The filter cake was purified by prep-HPLC (column: Phenomenex luna C18 150×40 mm×15 μm; mobile phase: [water(TFA)-ACN]; gradient: 25%-55% B over 10 min) and lyophilized to afford the impure product, which was further purified by prep-HPLC (column: Waters Xbridge C18 150×50 mm×10 μm; mobile phase: [water(NH₄HCO₃)-ACN]; gradient: 25%-55% B over 10 min) and lyophilized to afford N-(2-chlorophenyl)-4-((2-((4-((2-((1-(4-(2,6-dioxopiperidin-3-yl)-2-fluorophenyl)piperidin-4-yl)methyl)-2-azaspiro[3.3]heptan-6-yl)carbamoyl)phenyl)amino)-5-fluoropyrimidin-4-yl)amino) benzamide (64.8 mg, 71.81 μmol, 25.40% yield, 96.9% purity) as a white solid. MS(M+H)⁺=874.4.

¹H NMR (400 MHz, DMSO-d₆) δ=10.80 (s, 1H), 9.92 (s, 1H), 9.73 (s, 1H), 9.60 (s, 1H), 8.34 (d, J=7.5 Hz, 1H), 8.25 (d, J=3.5 Hz, 1H), 8.07-7.97 (m, 4H), 7.81-7.71 (m, 4H), 7.66 (dd, J=1.3, 7.9 Hz, 1H), 7.57 (dd, J=1.3, 7.9 Hz, 1H), 7.43-7.36 (m, 1H), 7.33-7.26 (m, 1H), 7.04-6.90 (m, 3H), 4.32-4.20 (m, 1H), 3.79 (dd, J=4.9, 11.7 Hz, 1H), 3.17 (s, 2H), 3.05 (s, 2H), 2.65-2.55 (m, 3H), 2.54-2.52 (m, 1H), 2.49-2.44 (m, 2H), 2.41-2.34 (m, 2H), 2.26-2.22 (m, 2H), 2.21-2.10 (m, 3H), 2.03-1.95 (m, 1H), 1.75-1.65 (m, 2H), 1.41-1.31 (m, 1H), 1.30-1.19 (m, 2H).

### Example 78. Synthesis of N-(2-chlorophenyl)-4-[[2-[4-[[4-[[2-[4-(2,6-dioxo-3-piperidyl)-2-fluoro-phenyl]-2,7-diazaspiro[3.5]nonan-7-yl]methyl]-1-piperidyl]carbamoyl]anilino]-5-fluoro-pyrimidin-4-yl]amino]benzamide (Compound 78)

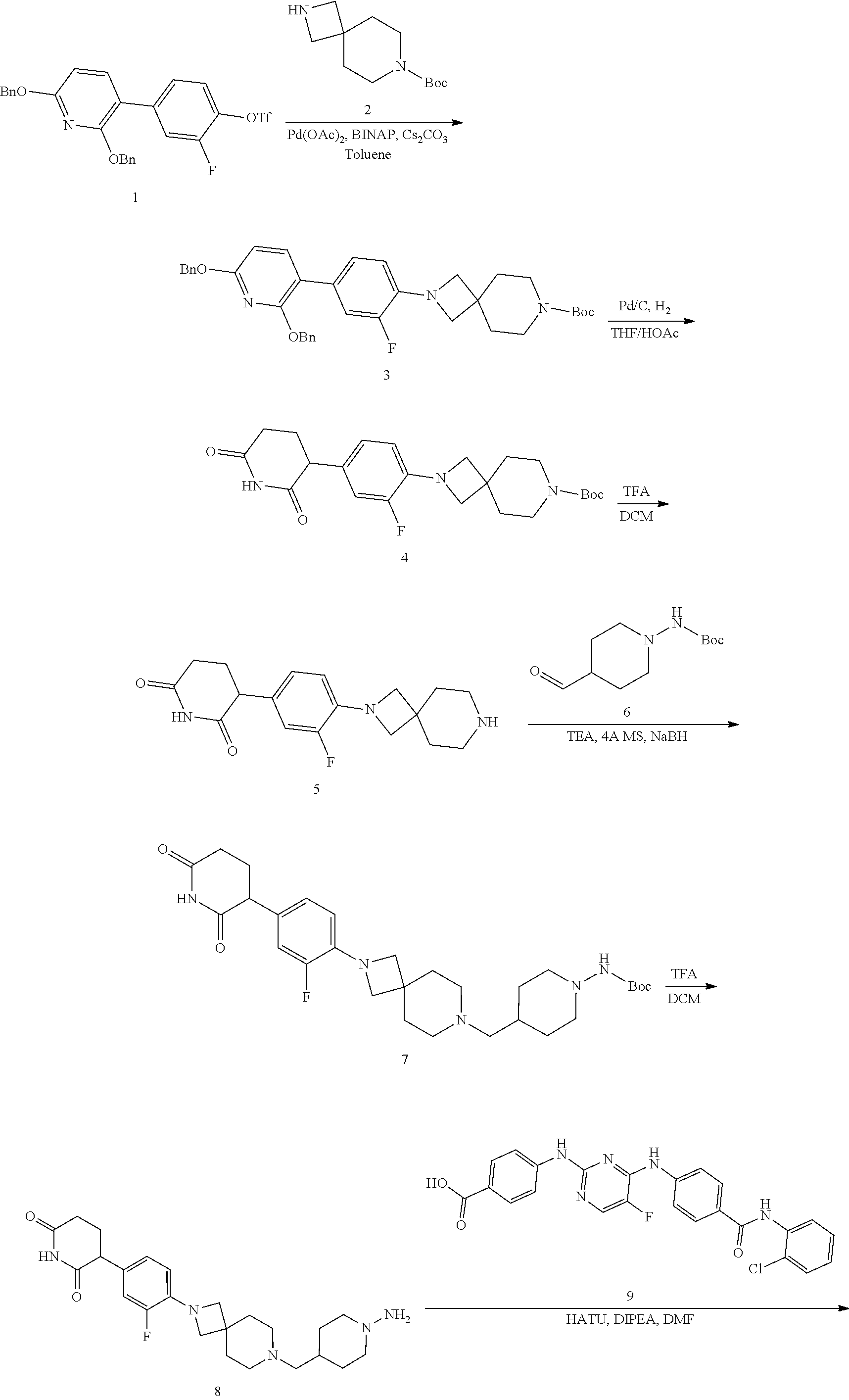

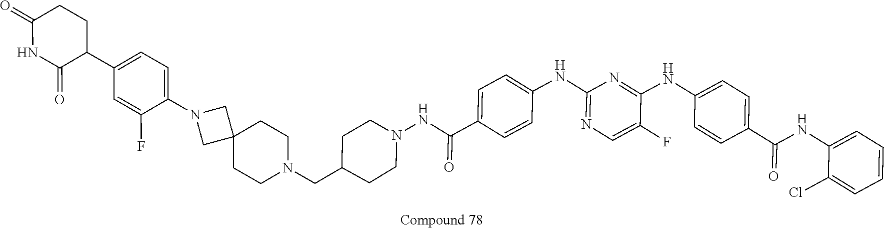 [SMILES: O=C1CCC(c2ccc(N3CC4(CCN(CC5CCN(NC(=O)c6ccc(Nc7ncc(F)c(Nc8ccc(C(=O)Nc9ccccc9Cl)cc8)n7)cc6)CC5)CC4)C3)c(F)c2)C(=O)N1]

### Step 1. Synthesis of tert-butyl 2-[4-(2,6-dibenzyloxy-3-pyridyl)-2-fluoro-phenyl]-2,7-diazaspiro[3.5]nonane-7-carboxylate (3)

A mixture of [4-(2,6-dibenzyloxy-3-pyridyl)-2-fluoro-phenyl] trifluoromethane-sulfonate (1 g, 1.87 mmol), tert-butyl 2, 7-diazaspiro[3.5]nonane-7-carboxylate (509.06 mg, 2.25 mmol), BINAP (116.72 mg, 187.44 μmol), Pd(OAc)₂ (42.08 mg, 187.44 μmol) and Cs₂CO₃ (1.83 g, 5.62 mmol) in toluene (10 mL) was degassed and purged with N₂ for 3 times, and then the mixture was stirred at 100° C. for 16 hr under N₂ atmosphere. LCMS showed a peak (45%) with desired mass. The reaction mixture was filtered and washed with EtOAc (20 mL). The filtrate was concentrated under reduced pressure to afford a residue, which was purified by flash silica gel chromatography (12 g SepaFlash® Silica Flash Column, Eluent of 0-30% EtOAc/Petroleum ether, gradient @ 60 mL/min) to afford tert-butyl 2-[4-(2, 6-dibenzyloxy-3-pyridyl)-2-fluoro-phenyl]-2,7-diazaspiro[3.5]nonane-7-carboxylate (1.3 g, 1.75 mmol, 93.27% yield, 82% purity) as yellow solid. MS(M+H)⁺=610.3.

¹H NMR (400 MHz, CDCl₃-d) δ=7.56 (d, J=8.1 Hz, 1H), 7.45-7.28 (m, 11H), 7.23 (dd, J=1.9, 6.4 Hz, 1H), 6.54-6.43 (m, 2H), 5.43 (s, 2H), 5.36 (s, 2H), 3.78-3.75 (m, 4H), 3.48-3.36 (m, 4H), 1.85-1.75 (m, 4H), 1.48 (s, 9H).

### Step 2. Synthesis of tert-butyl 2-[4-(2,6-dioxo-3-piperidyl-2-fluoro-phenyl]-2,7-diazaspiro[3.5]nonane-7-carboxylate (4)

To a solution of tert-butyl 2-[4-(2,6-dibenzyloxy-3-pyridyl)-2-fluoro-phenyl]-2,7-diazaspiro[3.5]nonane-7-carboxylate (1.3 g, 2.13 mmol) and HOAc (25.61 mg, 426.42 μmol, 24.41 μL) in THF (15 mL) was added Pd/C (226.90 mg, 213.21 μmol, 10% purity) under N₂ atmosphere. The suspension was degassed and purged with H₂ for 3 times. The mixture was stirred under H₂ (50 Psi) at 30° C. for 16 hr. TLC (Petroleum ether:EtOAc=1:1) showed one new spot was formed and starting material was consumed. The reaction mixture was filtered through celite pad and concentrated under reduced pressure to afford a residue. The residue was triturated with EtOAc and MeCN to afford tert-butyl 2-[4-(2,6-dioxo-3-piperidyl)-2-fluoro-phenyl]-2,7-diazaspiro[3.5]nonane-7-carboxylate (640 mg, 1.39 mmol, 65.39% yield, 94% purity) as white solid. MS(M+H)⁺=432.1.

¹H NMR (400 MHz, CDCl₃-d)=δ=7.95 (s, 1H), 6.82-6.70 (m, 2H), 6.37 (t, J=8.9 Hz, 1H), 3.66 (d, J=2.0 Hz, 4H), 3.60 (dd, J=5.1, 9.8 Hz, 1H), 3.36-3.29 (m, 4H), 2.71-2.51 (m, 2H), 2.24-2.05 (m, 2H), 1.75-1.65 (m, 4H), 1.39 (s, 9H).

### Step 3. Synthesis of 3-[4-(2,7-diazaspiro[3.5]nonan-2-yl)-3-fluoro-phenyl]piperidine-2,6-dione (5)

To a solution of tert-butyl 2-[4-(2,6-dioxo-3-piperidyl)-2-fluoro-phenyl]-2,7-diazaspiro[3.5]nonane-7-carboxylate (540 mg, 1.25 mmol) in DCM (10 mL) was added TFA (2.14 g, 18.77 mmol, 1.39 mL). The mixture was stirred at 20° C. for 1 hr. LCMS showed three peaks (91%) with the mass of desired product and starting material was consumed. The reaction mixture was concentrated under reduced pressure to afford 3-[4-(2, 7-diazaspiro[3.5]nonan-2-yl)-3-fluoro-phenyl]piperidine-2, 6-dione (550 mg, crude, TFA) as yellow oil. MS(M+H)⁺=332.2.

### Step 4. Synthesis of tert-butyl N-[4-[[2-[4-(2,6-dioxo-3-piperidyl)-2-fluoro-phenyl]-2,7-diazaspiro[3.5]nonan-7-yl]methyl]-1-piperidyl]carbamate (7)

To a solution of 3-[4-(2, 7-diazaspiro[3.5]nonan-2-yl)-3-fluoro-phenyl]piperidine-2, 6-dione (650 mg, 1.77 mmol, TFA) and tert-butyl N-(4-formyl-1-piperidyl) carbamate (484.07 mg, 2.12 mmol) in DCM (10 mL) were added 4 Å MS (300 mg) and TEA (894.03 mg, 8.84 mmol, 1.23 mL). After stirring at 20° C. for 30 min, NaBH(OAc)₃ (936.28 mg, 4.42 mmol) was added and the resulting mixture was stirred at 20° C. for 16 hr. LCMS showed a peak (83%) with desired mass. The reaction mixture was filtered. The filtrate was diluted with water (20 mL) and extracted with DCM (15 mL×3). The combined organic phase was concentrated under reduced pressure to afford a residue, which was triturated with EtOAc (5 mL) and MTBE (5 mL) to afford tert-butyl N-[4-[[2-[4-(2, 6-dioxo-3-piperidyl)-2-fluoro-phenyl]-2, 7-diazaspiro[3.5]nonan-7-yl]methyl]-1-piperidyl]carbamate (780 mg, 1.38 mmol, 77.94% yield, 96% purity) as white solid. MS(M+H)⁺=544.4.

### Step 5. Synthesis of 3-[4-[7-[(1-amino-4-piperidyl)methyl]-2,7-diazaspiro[3.5]nonan-2-yl]-3-fluoro-phenyl]piperidine-2,6-dione (8)

To a solution of tert-butyl N-[4-[[2-[4-(2, 6-dioxo-3-piperidyl)-2-fluoro-phenyl]-2, 7-diazaspiro[3.5]nonan-7-yl]methyl]-1-piperidyl]carbamate (200 mg, 367.87 μmol) in DCM (2 mL) was added TFA (629.18 mg, 5.52 mmol, 409.89 μL). The mixture was stirred at 20° C. for 2 hr. LCMS showed a peak (81%) with desired mass and starting material was consumed. The reaction mixture was concentrated under reduced pressure to afford 3-[4-[7-[(1-amino-4-piperidyl)methyl]-2,7-diazaspiro[3.5]nonan-2-yl]-3-fluoro-phenyl]piperidine-2,6-dione (200 mg, crude, TFA) as yellow oil. MS(M+H)⁺=444.3.

### Step 6. Synthesis of N-(2-chlorophenyl)-4-[[2-[4-[[4-[[2-[4-(2,6-dioxo-3-piperidyl-2-fluoro-phenyl-2,7-diazaspiro[3.5]nonan-7-yl]methyl]-1-piperidyl]carbamoyl]anilino]-5-fluoro-pyrimidin-4-yl]amino]benzamide (Compound 78)

To a solution of 3-[4-[7-[(1-amino-4-piperidyl)methyl]-2,7-diazaspiro[3.5]nonan-2-yl]-3-fluoro-phenyl]piperidine-2, 6-dione (200 mg, 358.69 mol, TFA) and 4-[[4-[4-[(2-chlorophenyl)carbamoyl]anilino]-5-fluoro-pyrimidin-2-yl]amino]benzoic acid (171.41 mg, 358.69 μmol) in DMF (3 mL) were added HATU (204.58 mg, 538.04 mol) and DIPEA (139.08 mg, 1.08 mmol, 187.43 μL). The resulting mixture was stirred at 20° C. for 16 hr. LCMS showed a peak (23%) with desired product. The reaction mixture was diluted with water (20 mL) and filtered. The filter cake was purified by prep-HPLC (column: Phenomenex luna C18 150*25 mm*10 um; mobile phase: [water(TFA)-ACN]; gradient: 27%-57% B over 10 min) and lyophilized to afford N-(2-chlorophenyl)-4-[[2-[4-[[4-[[2-[4-(2,6-dioxo-3-piperidyl)-2-fluoro-phenyl]-2,7-diazaspiro[3.5]nonan-7-yl]methyl]-1-piperidyl]carbamoyl]anilino]-5-fluoro-pyrimidin-4-yl]amino]benzamide (9.7 mg, 9.99 μmol, 2.78% yield, 93% purity) as white solid. MS(M+H)⁺=903.4.

¹H NMR (400 MHz, DMSO-d₆) δ=10.78 (s, 1H), 9.93 (s, 1H), 9.75 (s, 1H), 9.63 (s, 1H), 9.45-9.28 (m, 1H), 8.84-8.74 (m, 1H), 8.25 (d, J=3.4 Hz, 1H), 8.06-7.97 (m, 4H), 7.78 (br d, J=8.9 Hz, 2H), 7.74-7.68 (m, 2H), 7.65 (d, J=6.8 Hz, 1H), 7.59-7.53 (m, 1H), 7.45-7.36 (m, 1H), 7.35-7.27 (m, 1H), 6.99-6.92 (m, 1H), 6.89 (br d, J=7.8 Hz, 1H), 6.53-6.44 (m, 1H), 3.90-7.80 (m, 4H), 3.75-3.65 (m, 2H), 3.09-2.91 (m, 6H), 2.85-2.72 (m, 2H), 2.23-2.07 (m, 4H), 2.04-1.86 (m, 4H), 1.84-1.73 (m, 3H), 1.45-1.20 (m, 3H).

### Example 79. Synthesis of N-(2-chlorophenyl)-4-((2-((4-((4-((2R)-1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (Compound 79)

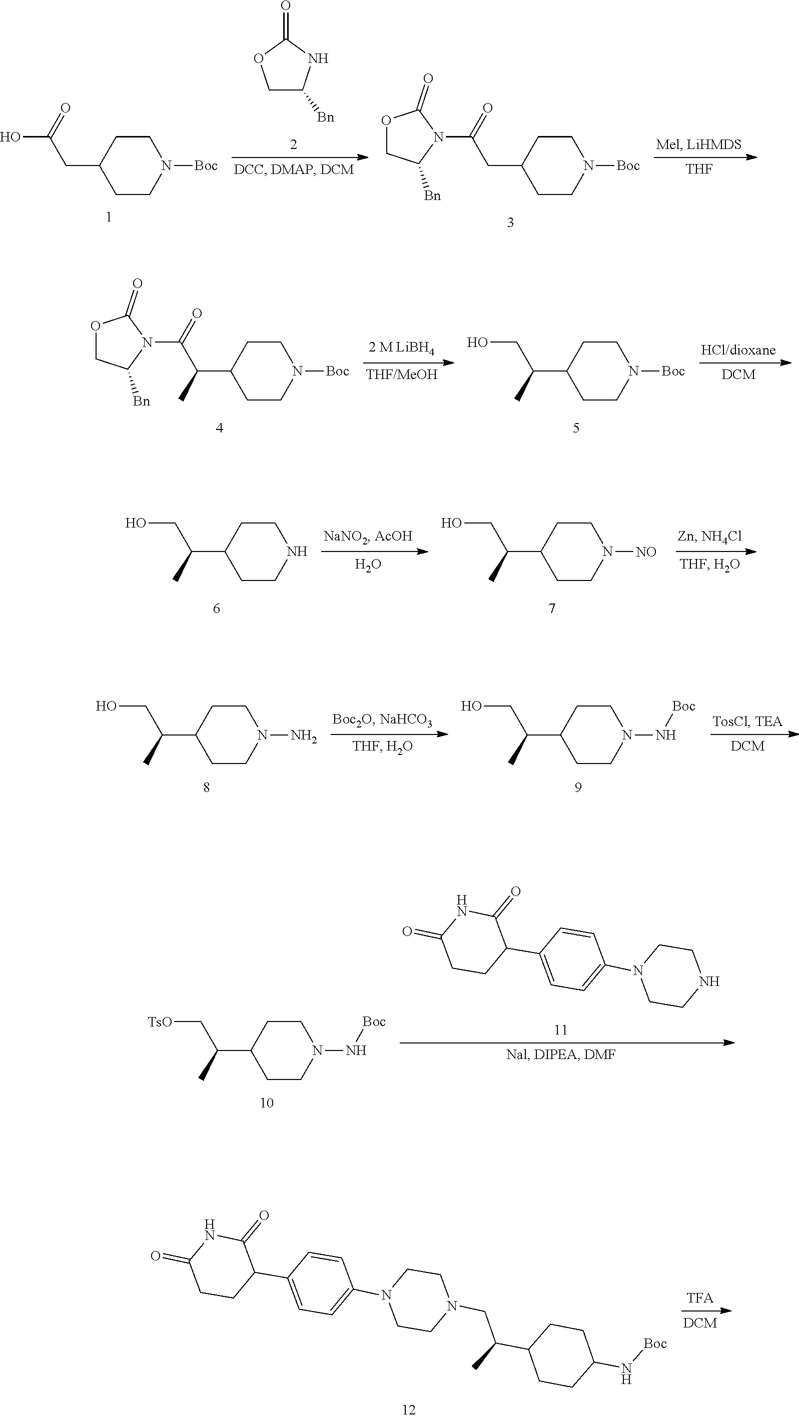

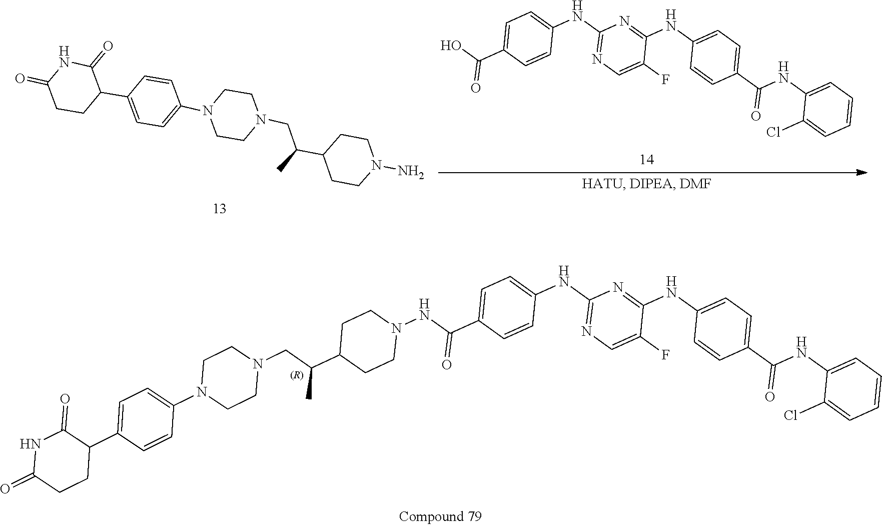

### Step 1. Synthesis of tert-butyl (R)-4-(2-(4-benzyl-2-oxooxazolidin-3-yl)-2-oxoethyl)piperidine-1-carboxylate (3)

To a solution of 2-(1-(tert-butoxycarbonyl)piperidin-4-yl)acetic acid (10 g, 41.10 mmol) and (R)-4-benzyloxazolidin-2-one (7.30 g, 41.20 mmol) in DCM (100 mL) was added DMAP (2.51 g, 20.55 mmol) and DCC (10.18 g, 49.32 mmol, 9.98 mL) and the mixture was stirred at 20° C. for 14 h. LCMS showed the desired mass. The mixture was filtered and the filter cake was washed with DCM (100 mL). The filtrate was concentrated under reduced pressure and then purified by flash silica gel chromatography (120 g SepaFlash® Silica Flash Column, Eluent of 20-30% EtOAc/Petroleum ether gradient @ 150 mL/min) to afford tert-butyl (R)-4-(2-(4-benzyl-2-oxooxazolidin-3-yl)-2-oxoethyl)piperidine-1-carboxylate (16.9 g, 38.63 mmol, 93.99% yield, 92% purity) as a yellow oil. MS(M−t−Bu+H)⁺=347.2.

### Step 2. Synthesis of tert-butyl 4-((R)-1-((R)-4-benzyl-2-oxooxazolidin-3-yl)-1-oxopropan-2-yl)piperidine-1-carboxylate (4)

To a solution of tert-butyl (R)-4-(2-(4-benzyl-2-oxooxazolidin-3-yl)-2-oxoethyl)piperidine-1-carboxylate (16.9 g, 41.99 mmol) in THF (170 mL) was slowly added LiHMDS (2 M, 42 mL) at 0° C. and the mixture stirred at 0° C. for 30 min. Then Mel (11.92 g, 83.98 mmol, 5.23 mL) was added at −40° C. and the mixture was stirred at −40° C. for 2 h, followed by stirring at 20° C. for 14 h. LCMS showed 42% of the desired mass. The mixture was quenched with NH₄Cl (150 mL) and extracted with EtOAc (100 mL). The combined organic layer was dried over Na₂SO₄, filtered and concentrated under reduced pressure. The residue was purified by flash silica gel chromatography (120 g SepaFlash® Silica Flash Column, Eluent of 20-50% EtOAc/Petroleum ether gradient @ 200 mL/min) to afford tert-butyl 4-((R)-1-((R)-4-benzyl-2-oxooxazolidin-3-yl)-1-oxopropan-2-yl)piperidine-1-carboxylate (7.5 g, 18.01 mmol, 42.88% yield) as a white solid. MS(M−t−Bu+H)⁺=361.1.

### Step 3. Synthesis of tert-butyl (R)-4-(1-hydroxypropan-2-yl)piperidine-1-carboxylate

To a solution of tert-butyl 4-((R)-1-((R)-4-benzyl-2-oxooxazolidin-3-yl)-1-oxopropan-2-yl)piperidine-1-carboxylate (2 g, 4.80 mmol) in THF (40 mL) and MeOH (1 mL) was slowly added LiBH₄ (2 M, 7.20 mL) at 0° C. under N₂ and the mixture was stirred at 20° C. under N₂ for 14 h. LCMS showed the starting material was consumed. The mixture was quenched with NH₄Cl (50 mL) and extracted with EtOAc (30 mL×2), the combined organic layer was dried over Na₂SO₄ and filtered. The filtrate was concentrated under reduced pressure to afford tert-butyl (R)-4-(1-hydroxypropan-2-yl)piperidine-1-carboxylate (1.87 g, crude) as a yellow oil. MS(M−t−Bu+H)⁺=188.3.

### Step 4. Synthesis of (R)-2-(piperidin-4-yl)propan-1-ol (6)

To a solution of tert-butyl (R)-4-(1-hydroxypropan-2-yl)piperidine-1-carboxylate (1.67 g, 6.86 mmol) in DCM (10 mL) was added HCl/dioxane (4 M, 40 mL) and the mixture was stirred at 25° C. 1 h. LCMS showed the starting material was consumed. The mixture was concentrated under reduced pressure to afford (R)-2-(piperidin-4-yl)propan-1-ol (1.3 g, crude, HCl) as a yellow oil. MS(M+H)⁺=144.3.

### Step 5. Synthesis of (R)-2-(1-nitrosopiperidin-4-yl)propan-1-ol (7)

To a solution of (R)-2-(piperidin-4-yl)propan-1-ol (1.3 g, 9.08 mmol) in H₂O (20 mL) was added NaNO₂ (2.50 g, 36.31 mmol) at 0° C. and then followed by AcOH (2.73 g, 45.42 mmol, 2.60 mL) and the mixture was stirred at 25° C. for 14 h. TLC (Petroleum ether:EtOAc=10:1) showed the new spot was detected. The mixture was combined with other batches (150 mg scale) and then adjusted the pH=8 with NaHCO₃ (solid). The mixture was extracted with EtOAc (20 mL×3) and EtOAc/MeOH=10/1 (30 mL×3), the combined organic layer was dried over Na₂SO₄ and filtered. The filtrate was concentrated under reduced pressure. The residue was purified by flash silica gel chromatography (12 g SepaFlash® Silica Flash Column, Eluent of 60~100% EtOAc/Petroleum ether gradient @ 80 mL/min) to afford (R)-2-(1-nitrosopiperidin-4-yl)propan-1-ol (870 mg, 5.05 mmol, 55.65% yield) as a yellow oil. MS(M+H)⁺=173.4.

### Step 6. Synthesis of (R)-2-(1-aminopiperidin-4-yl)propan-1-ol (8)

To a solution of (R)-2-(1-nitrosopiperidin-4-yl)propan-1-ol (870 mg, 5.05 mmol) and NH₄Cl (811 mg, 15.16 mmol) in THF (10 mL) and H₂O (5 mL) was slowly added Zn (1.51 g, 23.09 mmol) and the mixture was stirred at 25° C. for 1 h. LCMS showed the starting material was consumed and the desired mass was detected. The mixture was filtered and the filter cake was washed with THF (100 mL). The filtrate was concentrated under reduced pressure to afford (R)-2-(1-aminopiperidin-4-yl)propan-1-ol (1.5 g, crude) as a white solid. MS(M+H)⁺=159.3.

### Step 7. Synthesis of tert-butyl (R)-(4-(1-hydroxypropan-2-yl)piperidin-1-yl)carbamate (9)

To a solution of (R)-2-(1-aminopiperidin-4-yl)propan-1-ol (1.5 g, 9.48 mmol) in THF (30 mL) and H₂O (20 mL) was added NaHCO₃ (1.59 g, 18.96 mmol, 737.67 μL) and Boc₂O (4.14 g, 18.96 mmol, 4.36 mL) and the mixture was stirred at 25° C. for 14 h. LCMS showed 39% of the desired mass. The mixture was extracted with EtOAc (30 mL×3), the combined organic layer was dried over Na₂SO₄ and filtered. The filtrate was concentrated under reduced pressure and then purified by flash silica gel chromatography (5 g SepaFlash® Silica Flash Column, Eluent of 30-50% EtOAc/Petroleum ether gradient @ 100 mL/min) to afford tert-butyl (R)-(4-(1-hydroxypropan-2-yl)piperidin-1-yl)carbamate (810 mg, 3.14 mmol, 33.07% yield) as a yellow oil. MS(M+H)⁺=259.3.

### Step 8. Synthesis of (R)-2-(1-((tert-butoxycarbonyl)amino)piperidin-4-yl)propyl 4-methylbenzenesulfonate (10)

To a solution of tert-butyl (R)-(4-(1-hydroxypropan-2-yl)piperidin-1-yl)carbamate (810 mg, 3.14 mmol) in DCM (15 mL) were added TEA (951.75 mg, 9.41 mmol, 1.31 mL) and TosCl (777.03 mg, 4.08 mmol) at 0° C. and the mixture was stirred at 25° C. for 14 h. LCMS showed the desired mass. The mixture was diluted with water (20 mL) and extracted with EtOAc (20 mL×2), the combined organic layer was dried over Na₂ SO₄ and filtered. The filtrate was concentrated under reduced pressure. The residue was purified by flash silica gel chromatography (5 g SepaFlash® Silica Flash Column, Eluent of 20% EtOAc/Petroleum ether gradient @ 50 mL/min) to afford (R)-2-(1-((tert-butoxycarbonyl)amino)piperidin-4-yl)propyl 4-methylbenzenesulfonate (450 mg, 981.72 μmol, 31.31% yield, 90% purity) as a yellow oil. MS(M+H)⁺=413.3.

### Step 9. Synthesis of tert-butyl (4-((2R)-1-(4-(4-(2,6-dioxopiperidin-3-yl)phenyl)piperazin-1-yl)propan-2-yl)piperidin-1-yl)carbamate (12)

To a solution of 3-(4-(piperazin-1-yl)phenyl)piperidine-2,6-dione (280.00 mg, 903.84 μmol, HCl) in DMF (6 mL) were added DIPEA (356.16 mg, 2.76 mmol, 480.00 μL) and NaI (13.55 mg, 90.38 μmol) and then followed by (R)-2-(1-((tert-butoxycarbonyl)amino)piperidin-4-yl)propyl 4-methylbenzenesulfonate (0.4 g, 969.60 μmol). The resulting mixture was stirred at 60° C. for 14 h. LCMS showed the desired mass. The mixture was diluted with water (10 mL) and extracted with EtOAc (10 mL×3), the combined organic layer was washed with brine (10 mL), dried over Na₂SO₄ and filtered. The filtrate was concentrated under reduced pressure and then purified by flash silica gel chromatography (5 g SepaFlash® Silica Flash Column, Eluent of 70-100% EtOAc/Petroleum ether gradient @ 50 mL/min) to afford tert-butyl (4-((2R)-1-(4-(4-(2,6-dioxopiperidin-3-yl)phenyl)piperazin-1-yl)propan-2-yl)piperidin-1-yl)carbamate (45 mg, 87.60 μmol, 9.69% yield) as a yellow solid. MS(M+H)⁺=514.4.

### Step 10. Synthesis of 3-(4-(4-((R)-2-(1-aminopiperidin-4-yl)propyl)piperazin-1-yl)phenyl)piperidine-2,6-dione (13)

To a solution of tert-butyl (4-((2R)-1-(4-(4-(2,6-dioxopiperidin-3-yl)phenyl)piperazin-1-yl)propan-2-yl)piperidin-1-yl)carbamate (45 mg, 87.60 μmol) in DCM (1 mL) was added TFA (153.50 mg, 1.35 mmol, 0.1 mL) and the mixture was stirred at 25° C. for 40 min. LCMS showed the desired mass was detected. The mixture was concentrated under reduced pressure to afford 3-(4-(4-((R)-2-(1-aminopiperidin-4-yl)propyl)piperazin-1-yl)phenyl)piperidine-2,6-dione (50 mg, crude, TFA) as a yellow oil. MS(M+H)⁺=414.3.

### Step 11. Synthesis of N-(2-chlorophenyl)-4-((2-((4-((4-((2R)-1-(4-(4-(2,6-dioxopiperidin-3-yl)phenyl)piperazin-1-ylpropan-2-ylpiperidin-1-yl)carbamoyl)phenyl)amino)-5-fluoropyrimidin-4-yl)amino)benzamide (Compound 79)

To a solution of 4-((4-((4-((2-chlorophenyl)carbamoyl)phenyl)amino)-5-fluoropyrimidin-2-yl) amino)benzoic acid (40 mg, 83.70 μmol) and HATU (38.19 mg, 100.44 μmol) in DMF (1 mL) was added DIPEA (108.18 mg, 837.04 μmol, 145.80 μL) and the mixture was stirred at 25° C. for 15 min. Then a solution of 3-(4-(4-((R)-2-(1-aminopiperidin-4-yl)propyl)piperazin-1-yl)phenyl)piperidine-2,6-dione (50 mg, 94.77 mol, TFA) in DMF (1 mL) was added at 0° C. and the mixture was stirred at 25° C. for 30 min. LCMS showed 45% of the desired mass. The mixture was diluted with water (5 mL) and then filtered. The filter cake was washed with water (5 mL). The filter cake was collected and the solvent was removed under reduced pressure. The crude was purified by prep-HPLC (column: Phenomenex luna C18 150*25 mm*10 um; mobile phase: [water (TFA)-ACN]; gradient: 26%-56% B over 10 min) and the eluent was lyophilized to afford N-(2-chlorophenyl)-4-((2-((4-((4-((2R)-1-(4-(4-(2,6-dioxopiperidin-3-yl)phenyl)piperazin-1-yl)propan-2-yl)piperidin-1-yl)carbamoyl)phenyl)amino)-5-fluoropyrimidin-4-yl)amino)benzamide (29 mg, 23.38 mol, 27.93% yield, 98% purity, 3TFA) as a yellow solid. MS(M+H)⁺=873.4.

¹H NMR (400 MHz, DMSO-d₆) δ=10.79 (s, 1H), 9.93 (s, 1H), 9.77 (s, 1H), 9.75-9.52 (m, 2H), 9.31-9.08 (m, 1H), 8.25 (d, J=3.5 Hz, 1H), 8.05-7.98 (m, 4H), 7.82-7.76 (m, 2H), 7.75-7.69 (m, 2H), 7.67-7.62 (m, 1H), 7.60-7.53 (m, 1H), 7.43-7.37 (m, 1H), 7.33-7.27 (m, 1H), 7.12 (d, J=8.7 Hz, 2H), 6.98 (d, J=8.7 Hz, 2H), 3.85-3.73 (m, 3H), 3.66-3.55 (m, 2H), 3.31-2.97 (m, 8H), 2.88-2.76 (m, 2H), 2.71-2.60 (m, 1H), 2.48-2.42 (m, 1H), 2.22-2.09 (m, 1H), 2.05-1.90 (m, 2H), 1.71-1.62 (m, 2H), 1.56-1.29 (m, 3H), 0.99 (br d, J=6.6 Hz, 3H).

### Example 80. Synthesis of N-(2-chlorophenyl)-4-((2-((4-(((1r,4r)-4-(((1-(4-(2,6-dioxopiperidin-3-yl)-2-fluorophenyl)piperidin-4-yl)methyl)(methyl)amino)cyclohexyl)carbamoyl)phenyl)amino)-5-fluoropyrimidin-4-yl)amino)benzamide (Compound 80)

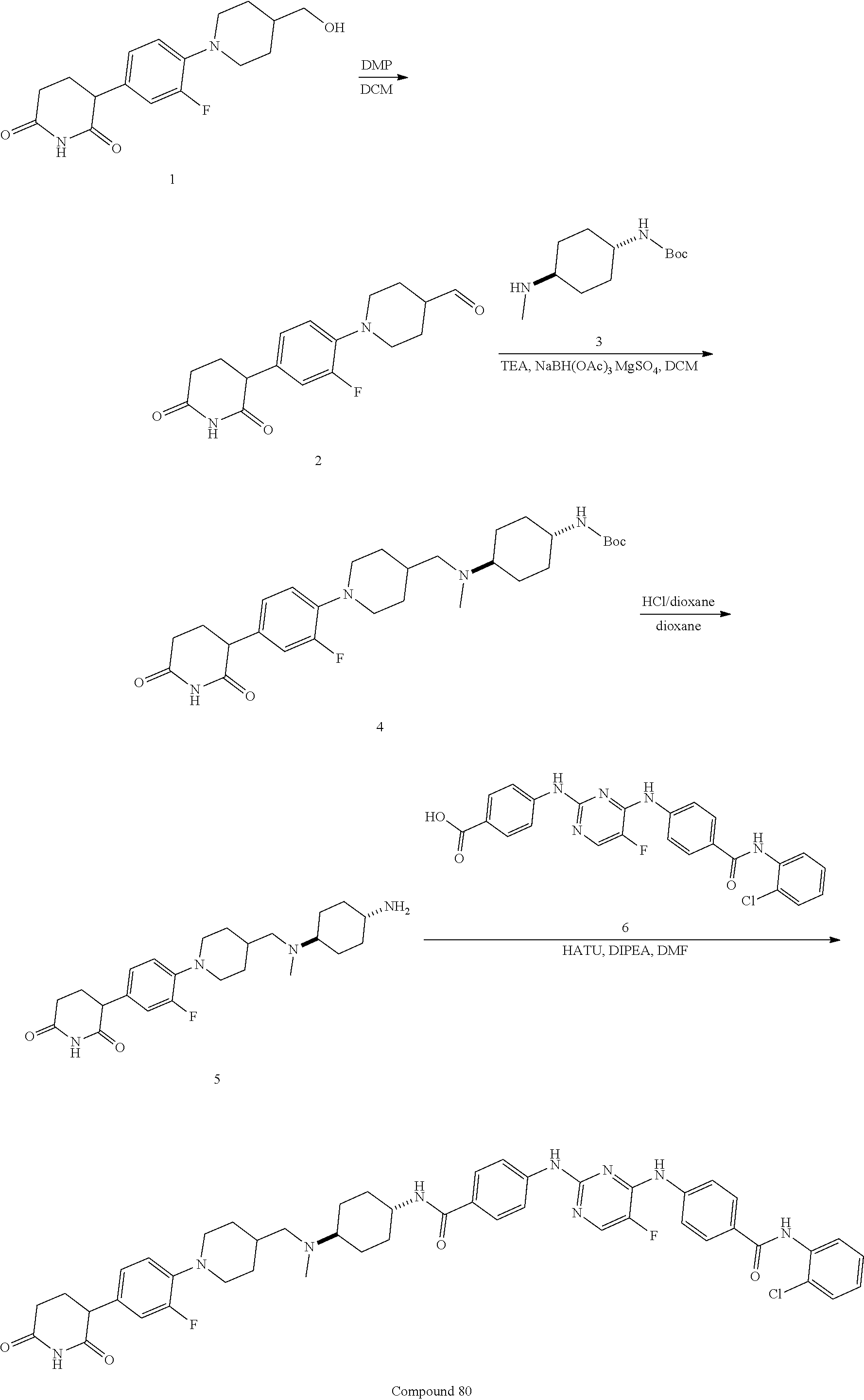

### Step 1. Synthesis of 1-(4-(2,6-dioxopiperidin-3-yl)-2-fluorophenyl)piperidine-4-carbaldehyde (2)

To a solution of 3-(3-fluoro-4-(4-(hydroxymethyl)piperidin-1-yl)phenyl)piperidine-2,6-dione (1 g, 3.12 mmol) in DCM (10 mL) was added DMP (1.46 g, 3.43 mmol). The mixture was stirred at 25° C. for 2 hr. TLC (petroleum ether:EtOAc=1:2) indicated 3-(3-fluoro-4-(4-(hydroxymethyl)piperidin-1-yl)phenyl)piperidine-2,6-dione was consumed completely and one new spot formed. The reaction mixture was washed with NaHCO₃ (10 mL×3), dried over Na₂SO₄, filtered and concentrated under reduced pressure to afford 1-(4-(2,6-dioxopiperidin-3-yl)-2-fluorophenyl)piperidine-4-carbaldehyde (990 mg, 3.11 mmol, 99.63% yield) as a gray solid, which was used directly. MS(M+H)⁺=319.4.

### Step 2. Synthesis of tert-butyl ((1r,4r)-4-(((1-(4-(2,6-dioxopiperidin-3-yl)-2-fluorophenyl)piperidin-4-yl)methyl)(methyl)amino)cyclohexyl)carbamate (4)

To a solution of tert-butyl ((1r,4r)-4-(methylamino)cyclohexyl)carbamate (250 mg, 1.09 mmol) and 1-(4-(2,6-dioxopiperidin-3-yl)-2-fluorophenyl)piperidine-4-carbaldehyde (487.98 mg, 1.53 mmol) in DCM (10 mL) were added TEA (110.79 mg, 1.09 mmol, 152.40 μL) and MgSO₄ (131.79 mg, 1.09 mmol). After stirring at 25° C. for 30 min, NaBH(OAc)₃ (696.16 mg, 3.28 mmol) was added and the resulting mixture was stirred at 25° C. for 16 hr. LCMS showed one main peak with desired mass was detected. The residue was diluted with NaHCO₃ solution (20 mL) and extracted with DCM (20 mL×3). The combined organic layers were washed with water (10 mL), dried over Na₂SO₄, filtered and concentrated under reduced pressure. The residue was purified by flash silica gel chromatography (ISCO®; 20 g SepaFlash® Silica Flash Column, Eluent of 20-30% MeOH/EtOAc gradient @ 80 mL/min) to afford tert-butyl ((1r,4r)-4-(((1-(4-(2,6-dioxopiperidin-3-yl)-2-fluorophenyl)piperidin-4-yl)methyl)(methyl)amino)cyclohexyl)carbamate (400 mg, 753.76 mol, 68.84% yield) was obtained as a white solid. MS(M+H)⁺=531.4.

### Step 3. Synthesis of 3-(4-(4-((((1r,4r)-4-aminocyclohexyl)(methyl)amino)methyl)piperidin-1-yl)-3-fluorophenyl)piperidine-2,6-dione (5)

To a solution of tert-butyl ((1r,4r)-4-(((1-(4-(2,6-dioxopiperidin-3-yl)-2-fluorophenyl)piperidin-4-yl)methyl)(methyl)amino)cyclohexyl)carbamate (400 mg, 753.76 μmol) in dioxane (4 mL) was added HCl/dioxane (4 M, 4 mL). The mixture was stirred at 25° C. for 2 hr. LCMS showed one main peak with desired mass was detected. The reaction mixture was concentrated under reduced pressure to afford 3-(4-(4-((((1r,4r)-4-aminocyclohexyl)(methyl)amino)methyl)piperidin-1-yl)-3-fluorophenyl)piperidine-2,6-dione (352 mg, 753.72 mol, 99.99% yield, HCl salt) as a white solid. MS(M+H)⁺=431.2.

### Step 4. Synthesis of N-(2-chlorophenyl)-4-((2-((4-(((1r,4r)-4-(((1-(4-(2,6-dioxopiperidin-3-yl)-2-fluorophenyl)piperidin-4-yl)methyl)(methyl)amino)cyclohexyl)carbamoyl)phenyl)amino)-5-fluoropyrimidin-4-yl)amino)benzamide (Compound 80)

To a solution of 4-((4-((4-((2-chlorophenyl)carbamoyl)phenyl)amino)-5-fluoropyrimidin-2-yl) amino)benzoic acid (300 mg, 566.26 μmol) and HATU (322.96 mg, 849.39 μmol) in DMF (5 mL) was added and DIPEA (365.92 mg, 2.83 mmol, 493.15 μL), followed by the addition of 3-(4-(4-((((1r,4r)-4-aminocyclohexyl)(methyl)amino)methyl)piperidin-1-yl)-3-fluorophenyl)piperidine-2,6-dione (317.34 mg, 679.51 μmol, HCl salt). The resulting mixture was stirred at 25° C. for 2 hr. LCMS showed one main peak with desired mass was detected. The reaction mixture was diluted with water (20 mL) and filtered. The filter cake was collected and dried. The crude product was purified by prep-HPLC (column: Phenomenex luna C18 150×40 mm×15 μm; mobile phase: [water(TFA)-ACN]; gradient: 25%-55% B over 10 min) and lyophilized to afford the impure product, which was further purified by prep-HPLC (column: Waters Xbridge C18 150×50 mm×10 μm; mobile phase: [water(NH₄HCO₃)-ACN]; gradient: 32%-62% B over 10 min) and lyophilized to afford N-(2-chlorophenyl)-4-((2-((4-(((1r,4r)-4-(((1-(4-(2,6-dioxopiperidin-3-yl)-2-fluorophenyl)piperidin-4-yl)methyl)(methyl)amino)cyclohexyl)carbamoyl)phenyl)amino)-5-fluoropyrimidin-4-yl)amino)benzamide (206.6 mg, 219.50 μmol, 38.76% yield, 94.6% purity) as a white solid. MS(M+H)⁺=890.4.

¹H NMR (400 MHz, DMSO-d₆) δ=10.81 (s, 1H), 9.93 (s, 1H), 9.74 (s, 1H), 9.59 (s, 1H), 8.24 (d, J=3.5 Hz, 1H), 8.05-7.98 (m, 4H), 7.96 (br d, J=7.8 Hz, 1H), 7.80-7.71 (m, 4H), 7.65 (dd, J=1.3, 7.9 Hz, 1H), 7.56 (dd, J=1.3, 8.0 Hz, 1H), 7.43-7.36 (m, 1H), 7.32-7.26 (m, 1H), 7.03-6.95 (m, 2H), 6.95-6.90 (m, 1H), 3.79 (dd, J=4.9, 11.8 Hz, 1H), 3.74-3.63 (m, 1H), 2.70-2.58 (m, 3H), 2.58-2.51 (m, 2H), 2.49-2.45 (m, 1H), 2.35-2.29 (m, 1H), 2.28-2.23 (m, 2H), 2.19 (s, 3H), 2.18-2.12 (m, 1H), 2.04-1.95 (m, 1H), 1.93-1.84 (m, 2H), 1.83-1.77 (m, 2H), 1.76-1.65 (m, 2H), 1.57-1.45 (m, 1H), 1.39-1.27 (m, 4H), 1.27-1.16 (m, 2H).

### Example 81. Synthesis of N-(2-chlorophenyl)-4-((2-((4-((4-(((1-(4-(2,6-dioxopiperidin-3-yl)-2-fluorophenyl)piperidin-4-yl)methyl)(methyl)amino)piperidin-1-yl)carbamoyl)phenyl) amino)-5-fluoropyrimidin-4-yl)amino)benzamide (Compound 81)

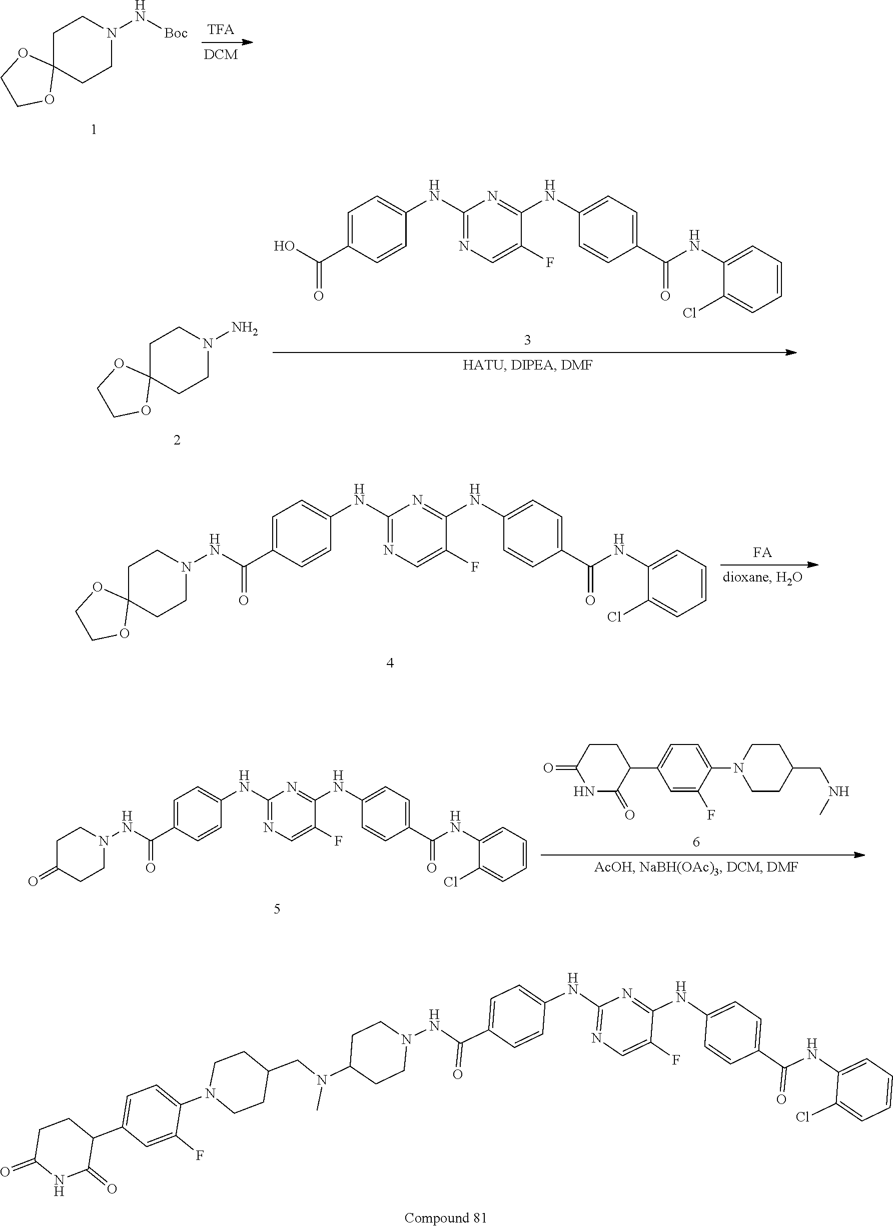

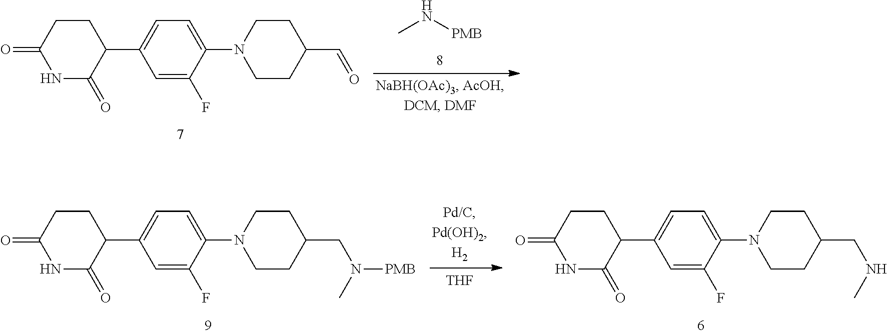

### Step 1. Synthesis of 1,4-dioxa-8-azaspiro[4.5]decan-8-amine (2)

To a solution of tert-butyl (1,4-dioxa-8-azaspiro[4.5]decan-8-yl)carbamate (330 mg, 1.28 mmol) in DCM (3 mL) was added TFA (1.54 g, 13.46 mmol, 1 mL). The mixture was stirred at 25° C. for 1 hr. LCMS showed tert-butyl (1,4-dioxa-8-azaspiro[4.5]decan-8-yl)carbamate was consumed completely and desired mass was detected. The reaction mixture was concentrated under reduced pressure to afford 1,4-dioxa-8-azaspiro[4.5]decan-8-amine (340 mg, crude, TFA) as a red oil. MS(M+55)+=159.1.

### Step 2. Synthesis of 4-((2-((4-((1,4-dioxa-8-azaspiro[4.5]decan-8-yl)carbamoyl)phenyl)amino)-5-fluoropyrimidin-4-yl)amino)-N-(2-chlorophenyl)benzamide (4)

To a solution of 4-((4-((4-((2-chlorophenyl)carbamoyl)phenyl)amino)-5-fluoropyrimidin-2-yl) amino)benzoic acid (537.17 mg, 1.12 mmol) in DMF (3 mL) were added HATU (712.35 mg, 1.87 mmol) and DIPEA (807.11 mg, 6.24 mmol, 1.09 mL). After stirring at 25° C. for 0.5 hr, 1,4-dioxa-8-azaspiro[4.5]decan-8-amine (340 mg, 1.25 mmol, TFA) was added and the resulting mixture was stirred at 25° C. for 1 hr. LCMS showed 1,4-dioxa-8-azaspiro[4.5]decan-8-amine was consumed completely and desired mass was detected. The reaction mixture was diluted water (20 mL) and extracted with EtOAc (10 mL×3). The combined organic layers were washed with brine (20 mL), dried over Na₂SO₄, filtered and concentrated under reduced pressure. The residue was purified by flash silica gel chromatography (ISCO®; 20 g SepaFlash® Silica Flash Column, Eluent of 0~20% MeOH/EtOAc @ 80 mL/min) to afford 4-((2-((4-((1,4-dioxa-8-azaspiro[4.5]decan-8-yl)carbamoyl)phenyl)amino)-5-fluoropyrimidin-4-yl)amino)-N-(2-chlorophenyl)benzamide (260 mg, 420.67 μmol, 33.68% yield) as a white solid. MS(M+H)⁺=618.2.

### Step 3. Synthesis of N-(2-chlorophenyl)-4-((5-fluoro-2-((4-((4-oxopiperidin-1-yl)carbamoyl)phenyl) amino)pyrimidin-4-yl)amino)benzamide (5)

To a solution of 4-((2-((4-((1,4-dioxa-8-azaspiro[4.5]decan-8-yl)carbamoyl)phenyl)amino)-5-fluoropyrimidin-4-yl)amino)-N-(2-chlorophenyl)benzamide (200 mg, 323.59 μmol) in FA (6 mL) was added dioxane (1 mL) and H₂O (1 mL). The resulting mixture was stirred at 25° C. for 57 hr. LCMS showed the starting material was consumed completely and desired mass was detected. The reaction mixture was diluted with H₂O (20 mL) and neutralized by saturated Na₂CO₃ solution. The resulting mixture extracted with EtOAc (20 mL×2). The combined organic layers were dried over Na₂SO₄, filtered and concentrated under reduced pressure to afford N-(2-chlorophenyl)-4-((5-fluoro-2-((4-((4-oxopiperidin-1-yl)carbamoyl)phenyl)amino)pyrimidin-4-yl)amino)benzamide (130 mg, crude) as a white solid. MS(M+H)⁺=574.1.

### Step 4. Synthesis of 3-(3-fluoro-4-(4-(((4-methoxybenzyl)(methyl)amino)methyl)piperidin-1-yl)phenyl)piperidine-2,6-dione (9)

To a solution of 1-(4-methoxyphenyl)-N-methylmethanamine (142.49 mg, 942.38 mol) in DCM (3 mL) was added the solution of 1-(4-(2,6-dioxopiperidin-3-yl)-2-fluorophenyl)piperidine-4-carbaldehyde (300 mg, 942.38 μmol) in DMF (1.5 mL), followed by the addition of AcOH (56.59 mg, 942.38 μmol, 53.95 μL). After stirring at 25° C. for 0.5 hr, NaBH(OAc)₃ (599.19 mg, 2.83 mmol) was added and the resulting mixture was stirred at 25° C. for 16 hr. LCMS showed 1-(4-(2,6-dioxopiperidin-3-yl)-2-fluorophenyl)piperidine-4-carbaldehyde was consumed completely and desired mass was detected. The reaction mixture was diluted with saturated Na₂CO₃ solution (30 mL) and extracted with EtOAc (10 mL×3). The combined organic layers were washed with brine (10 mL), dried over Na₂SO₄, filtered and concentrated under reduced pressure to give a residue. The residue was purified by flash silica gel chromatography (ISCO®; 20 g SepaFlash® Silica Flash Column, Eluent of 70-100% EtOAc/Petroleum ether, gradient @ 80 mL/min) to afford 3-(3-fluoro-4-(4-(((4-methoxybenzyl)(methyl)amino)methyl)piperidin-1-yl)phenyl)piperidine-2,6-dione (140 mg, 308.68 mol, 32.76% yield) as a white solid. MS(M+H)⁺=454.2.

### Step 5. Synthesis of 3-(3-fluoro-4-(4-((methylamino)methyl)piperidin-1-yl)phenyl)piperidine-2,6-dione (6)

To a solution of 3-(3-fluoro-4-(4-(((4-methoxybenzyl)(methyl)amino)methyl)piperidin-1-yl)phenyl)piperidine-2,6-dione (140 mg, 308.68 μmol) in THF (5 mL) were added Pd(OH)₂/C (50 mg, 20% purity) and Pd/C (50 mg, 10% purity) under N₂. The suspension was degassed under vacuum and purged with H₂ several times. The mixture was stirred under H₂ (40 psi) at 25° C. for 32 hours. LCMS showed 17% of 3-(3-fluoro-4-(4-(((4-methoxybenzyl)(methyl)amino) methyl)piperidin-1-yl)phenyl)piperidine-2,6-dione remained and the desired mass. The reaction mixture was filtered and washed THF (100 mL). The filtrate was concentrated under reduced pressure to afford 3-(3-fluoro-4-(4-((methylamino)methyl)piperidin-1-yl)phenyl)piperidine-2,6-dione (102 mg, crude) as a white solid. MS(M+H)⁺=334.1.

### Step 6. Synthesis of N-(2-chlorophenyl)-4-((2-((4-((4-(((1-(4-(2,6-dioxopiperidin-3-yl)-2-fluorophenyl)piperidin-4-yl)methyl)(methyl)amino)piperidin-1-yl)carbamoyl)phenyl) amino)-5-fluoropyrimidin-4-yl)amino)benzamide (Compound 81)

To a solution of 3-(3-fluoro-4-(4-((methylamino)methyl)piperidin-1-yl)phenyl)piperidine-2,6-dione (80 mg, 239.95 μmol) in DCM (1 mL) was added the solution NWO (2-chlorophenyl)-4-((5-fluoro-2-((4-((4-oxopiperidin-1-yl)carbamoyl)phenyl)amino)pyrimidin-4-yl)amino)benzamide (68.87 mg, 119.98 μmol) in DMF (0.5 mL) and AcOH (14.41 mg, 239.95 mol, 13.74 μL). After stirring at 25° C. for 0.5 hr, NaBH(OAc)₃ (152.57 mg, 719.86 μmol) was added and the resulting mixture was stirred at 25° C. for 20.5 hr. LCMS showed trace of the starting material remained and the desired mass. The reaction mixture was diluted water (20 mL) and extracted with EtOAc (10 mL×3). The combined organic layers were washed with brine (10 mL), dried over Na₂SO₄, filtered and concentrated under reduced pressure. The residue was purified by flash silica gel chromatography (ISCO®; 12 g SepaFlash® Silica Flash Column, Eluent of 0-30% EtOAc/Petroleum ether, gradient @ 60 mL/min). The impure product was purified by prep-HPLC (column: Waters Xbridge 150*25 mm*5 um; mobile phase: [water (NH₄HCO₃)-ACN]; gradient: 34%-64% B over 53 min) and lyophilized to afford N-(2-chlorophenyl)-4-((2-((4-((4-(((1-(4-(2,6-dioxopiperidin-3-yl)-2-fluorophenyl)piperidin-4-yl)methyl)(methyl)amino)piperidin-1-yl)carbamoyl)phenyl)amino)-5-fluoropyrimidin-4-yl)amino) benzamide (11.8 mg, 12.84 mol, 5.35% yield, 97% purity) as a white solid. MS(M+H)⁺=891.1.

¹H NMR (400 MHz, DMSO-d₆) δ=10.80 (s, 1H), 9.93 (s, 1H), 9.74 (s, 1H), 9.60 (s, 1H), 9.15 (s, 1H), 8.24 (d, J=3.5 Hz, 1H), 8.06-7.97 (m, 4H), 7.80-7.73 (m, 2H), 7.73-7.63 (m, 3H), 7.59-7.54 (m, 1H), 7.43-7.36 (m, 1H), 7.33-7.26 (m, 1H), 7.04-6.91 (m, 3H), 3.83-3.76 (m, 1H), 3.07-2.97 (m, 2H), 2.73 (br t, J=10.9 Hz, 2H), 2.68-2.57 (m, 3H), 2.50-2.46 (m, 2H), 2.35-2.25 (m, 3H), 2.23-2.13 (m, 4H), 2.05-1.95 (m, 1H), 1.85-1.75 (m, 2H), 1.71-1.63 (m, 2H), 1.62-1.48 (m, 3H), 1.32-1.17 (m, 3H).

### Example 82. Synthesis of N-(2-chlorophenyl)-4-((2-((4-((4-(2-(4-(4-(2,6-dioxopiperidin-3-yl)-3-fluorophenyl)piperazin-1-yl)ethyl)piperidin-1-yl)carbamoyl)phenyl)amino)-5-fluoropyrimidin-4-yl)amino)benzamide (Compound 82)

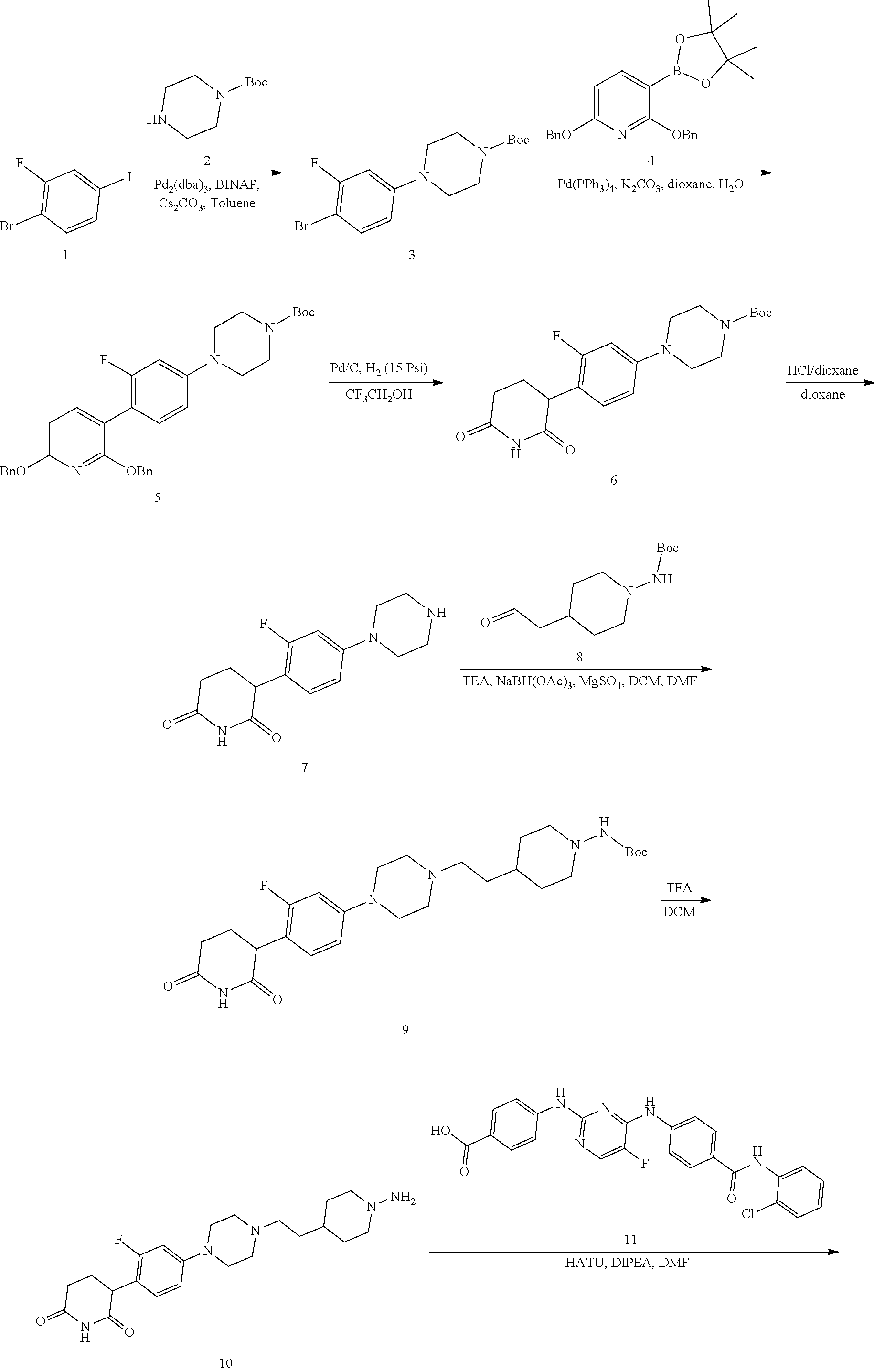

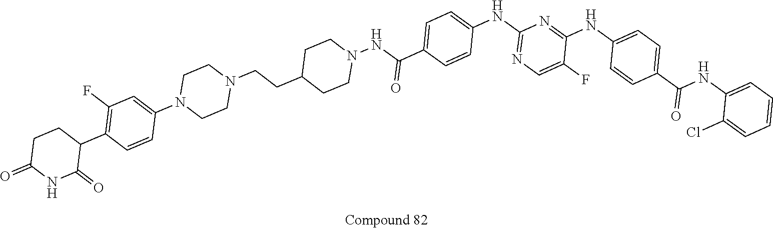 [SMILES: O=C1CCC(c2ccc(N3CCN(CCC4CCN(NC(=O)c5ccc(Nc6ncc(F)c(Nc7ccc(C(=O)Nc8ccccc8Cl)cc7)n6)cc5)CC4)CC3)cc2F)C(=O)N1]

### Step 1. Synthesis of tert-butyl 4-(4-bromo-3-fluorophenyl)piperazine-1-carboxylate (3)

A mixture of 1-bromo-2-fluoro-4-iodobenzene (5 g, 16.62 mmol), tert-butyl piperazine-1I-carboxylate (3.09 g, 16.62 mmol), Pd₂(dba)₃ (1.52 g, 1.66 mmol), Cs₂CO₃ (16.24 g, 49.85 mmol) and BINAP (1.03 g, 1.66 mmol) in toluene (50 mL) was degassed and purged with N, for 3 times, and then the mixture was stirred at 80° C. for 16 hr under N₂ atmosphere. LCMS showed 1-bromo-2-fluoro-4-iodobenzene was consumed completely desired mass was detected. The mixture was filtered and washed with EtOAc (50 mL). The filtrate was concentrated under reduced pressure. The residue was purified by flash silica gel chromatography (ISCO®; 80 g SepaFlash® Silica Flash Column, Eluent of 0-5% EtOAc/Petroleum ether, gradient @ 100 mL/min) to afford tert-butyl 4-(4-bromo-3-fluorophenyl)piperazine-1-carboxylate (2.75 g, 7.66 mmol, 46.07% yield) as a yellow solid. MS(M−55)+=303.1.

### Step 2. Synthesis of tert-butyl 4-(4-(2,6-bis(benzyloxy)pyridin-3-yl)-3-fluorophenyl)piperazine-1-carboxylate (5)

A mixture of tert-butyl 4-(4-bromo-3-fluorophenyl)piperazine-1-carboxylate (2 g, 5.57 mmol), 2,6-bis(benzyloxy)-3-(4,4,5,5-tetramethyl-1,3,2-dioxaborolan-2-yl)pyridine (3.02 g, 7.24 mmol), Pd(PPh₃)₄ (643.35 mg, 556.74 μmol) and K₂CO₃ (1.54 g, 11.13 mmol) in dioxane (20 mL) and H₂O (4 mL) was degassed and purged with N₂ for 3 times, and then the mixture was stirred at 100° C. for 16 hr under N₂ atmosphere. LCMS showed tert-butyl 4-(4-bromo-3-fluorophenyl)piperazine-1-carboxylate was consumed completely and desired mass was detected. The reaction mixture was diluted with EtOAc (100 mL), dried over Na₂SO₄, filtered and concentrated under reduced pressure. The residue was purified by flash silica gel chromatography (ISCO®; 40 g SepaFlash® Silica Flash Column, Eluent of 0-10% EtOAc/Petroleum ether, gradient @ 100 mL/min) to afford tert-butyl 4-(4-(2,6-bis(benzyloxy)pyridin-3-yl)-3-fluorophenyl)piperazine-1-carboxylate (2.77 g, 4.86 mmol, 87.34% yield) as a yellow oil. MS(M+H)⁺=570.3.

### Step 3. Synthesis of tert-butyl 4-(4-(2,6-dioxopiperidin-3-yl)-3-fluorophenyl)piperazine-1-carboxylate (6)

To a mixture of Pd/C (1 g, 10% purity) in CF₃CH₂OH (30 mL) under N₂ atmosphere was added tert-butyl 4-(4-(2,6-bis(benzyloxy)pyridin-3-yl)-3-fluorophenyl)piperazine-1-carboxylate (2.77 g, 4.86 mmol) at 25° C. The suspension was degassed under vacuum and purged with H₂ three times. The mixture was stirred under H₂ (15 Psi) at 25° C. for 16 h. LCMS showed tert-butyl 4-(4-(2,6-bis(benzyloxy)pyridin-3-yl)-3-fluorophenyl)piperazine-1-carboxylate was consumed completely and desired mass was detected. The mixture was filtered and washed with THF (250 mL). The filtrate was concentrated under reduced pressure to afford tert-butyl 4-(4-(2,6-dioxopiperidin-3-yl)-3-fluorophenyl)piperazine-1-carboxylate (1.58 g, crude) as a white solid. MS(M+H)⁺=392.2.

### Step 4. Synthesis of 3-(2-fluoro-4-(piperazin-1-yl)phenyl)piperidine-2,6-dione (7)

To a solution of tert-butyl 4-(4-(2,6-dioxopiperidin-3-yl)-3-fluorophenyl)piperazine-1-carboxylate (1.58 g, 4.04 mmol) in dioxane (16 mL) was added HCl/dioxane (4 M, 32 mL). The mixture was stirred at 25° C. for 4 hr. LCMS showed the starting material was consumed completely and desired mass was detected. The reaction mixture was concentrated under reduced pressure to afford 3-(2-fluoro-4-(piperazin-1-yl)phenyl)piperidine-2,6-dione (2.55 g, crude, HCl) as a white solid. MS(M+H)⁺=292.4.

### Step 5. Synthesis of tert-butyl (4-(2-(4-(4-(2,6-dioxopiperidin-3-yl)-3-fluorophenyl)piperazin-1-yl)ethyl)piperidin-1-yl)carbamate (9)

To a solution of 3-(2-fluoro-4-(piperazin-1-yl)phenyl)piperidine-2,6-dione (550 mg, 1.68 mmol, HCl) in DCM (6 mL) were added the solution of tert-butyl (4-(2-oxoethyl)piperidin-1-yl)carbamate (406.59 mg, 1.68 mmol) in DMF (2 mL), followed by the addition of TEA (848.95 mg, 8.39 mmol) and MgSO₄ (605.91 mg, 5.03 mmol). After stirring at 25° C. for 0.5 hr, NaBH (OAc)₃ (1.07 g, 5.03 mmol) was added and the resulting mixture was stirred at 25° C. for 16 hr. LCMS showed 3-(2-fluoro-4-(piperazin-1-yl)phenyl)piperidine-2,6-dione was consumed completely and desired mass was detected. The reaction mixture was diluted with H₂O (30 mL) and extracted with EtOAc (20 mL×2). The combined organic layers were washed with brine (20 mL), dried over Na₂SO₄, filtered and concentrated under reduced pressure. The residue was purified by flash silica gel chromatography (ISCO®; 20 g SepaFlash® Silica Flash Column, Eluent of 0-10% MeOH/EtOAc @ 100 mL/min) to afford tertbutyl(4-(2-(4-(4-(2,6-dioxopiperidin-3-yl)-3-fluorophenyl)piperazin-1-yl)ethyl)piperidin-1-yl)carbamate (0.2 g, 386.37 mol, 23.03% yield) as a white solid. MS(M+H)⁺=518.4.

### Step 6. Synthesis of 3-(4-(4-(2-(1-aminopiperidin-4-yl)ethyl)piperazin-1-yl)-2-fluorophenyl)piperidine-2,6-dione (10)

To a solution of tert-butyl (4-(2-(4-(4-(2,6-dioxopiperidin-3-yl)-3-fluorophenyl)piperazin-1-yl)ethyl)piperidin-1-yl)carbamate (0.2 g, 386.37 μmol) in DCM (2 mL) was added TFA (921.00 mg, 8.08 mmol, 0.6 mL). The mixture was stirred at 25° C. for 1 hr. LCMS showed the starting material was consumed completely and the desired mass. The reaction mixture was concentrated under reduced pressure to afford 3-(4-(4-(2-(1-aminopiperidin-4-yl)ethyl)piperazin-1-yl)-2-fluorophenyl)piperidine-2,6-dione (205 mg, crude, TFA) as a yellow oil. MS(M+H)⁺=418.3.

### Step 7. Synthesis of N-(2-chlorophenyl)-4-((2-((4-((4-(2-(4-(4-(2,6-dioxopiperidin-3-yl)-3-fluorophenyl)piperazin-1-ylethyl)piperidin-1-yl)carbamoyl)phenyl)amino)-5-fluoropyrimidin-4-yl)amino)benzamide (Compound 82)

To a solution of 4-((4-((4-((2-chlorophenyl)carbamoyl)phenyl)amino)-5-fluoropyrimidin-2-yl)amino)benzoic acid (140 mg, 292.96 μmol) in DMF (2 mL) were added HATU (167.09 mg, 439.45 μmol) and DIPEA (113.59 mg, 878.89 μmol, 153.09 μL). After stirring at 25° C. for 0.5 hr, 3-(4-(4-(2-(1-aminopiperidin-4-yl)ethyl)piperazin-1-yl)-2-fluorophenyl)piperidine-2,6-dione (202.44 mg, 380.85 μmol, TFA) was added and the resulting mixture was stirred at 25° C. for 16 hr. LCMS showed 3-(4-(4-(2-(1-aminopiperidin-4-yl)ethyl)piperazin-1-yl)-2-fluorophenyl)piperidine-2,6-dione was consumed completely and desired mass was detected. The reaction mixture was diluted with H₂O (20 mL) and extracted with EtOAc (10 mL×2). The combined organic layers were washed with brine (10 mL), dried over Na₂SO₄, filtered and concentrated under reduced pressure. The residue was purified by flash silica gel chromatography (ISCO®; 20 g SepaFlash® Silica Flash Column, Eluent of 0~20% EtOAc/MeOH @ 60 mL/min). The impure product was purified by prep-HPLC (column: Waters Xbridge 150*25 mm*5 um; mobile phase: [water (NH₄HCO₃)-ACN]; gradient: 37%-57% B over 53 min) and lyophilized to afford N-(2-chlorophenyl)-4-((2-((4-((4-(2-(4-(4-(2,6-dioxopiperidin-3-yl)-3-fluorophenyl)piperazin-1-yl)ethyl)piperidin-1-yl)carbamoyl)phenyl)amino)-5-fluoropyrimidin-4-yl)amino)benzamide (10.1 mg, 11.17 mol, 3.81% yield, 97% purity) as a white solid. MS(M+H)⁺=877.4.

¹H NMR (400 MHz, DMSO-d₆) δ=10.80 (br s, 1H), 9.93 (s, 1H), 9.74 (s, 1H), 9.60 (s, 1H), 9.12 (s, 1H), 8.24 (d, J=3.4 Hz, 1H), 8.06-7.97 (m, 4H), 7.80-7.73 (m, 2H), 7.72-7.62 (m, 3H), 7.56 (d, J=7.9 Hz, 1H), 7.39 (t, J=7.5 Hz, 1H), 7.33-7.25 (m, 1H), 7.08 (t, J=8.7 Hz, 1H), 6.76-6.67 (m, 2H), 3.93-3.85 (m, 1H), 3.52-3.38 (m, 3H), 3.35-3.30 (m, 2H), 3.20-3.10 (m, 4H), 2.97-2.90 (m, 2H), 2.76-2.63 (m, 3H), 2.38-2.29 (m, 2H), 2.21-2.07 (m, 1H), 2.01-1.89 (m, 1H), 1.70-1.60 (m, 2H), 1.45-1.38 (m, 2H), 1.35-1.19 (m, 3H).

### Example 83. Synthesis of 5-[[4-[4-[(2-chlorophenyl)carbamoyl]anilino]-5-fluoro-pyrimidin-2-yl]amino]-N-[4-[4-[4-(2,6-dioxo-3-piperidyl)phenyl]piperazin-1-yl]ethyl]-1-piperidyl]pyridine-2-carboxamide (Compound 83)

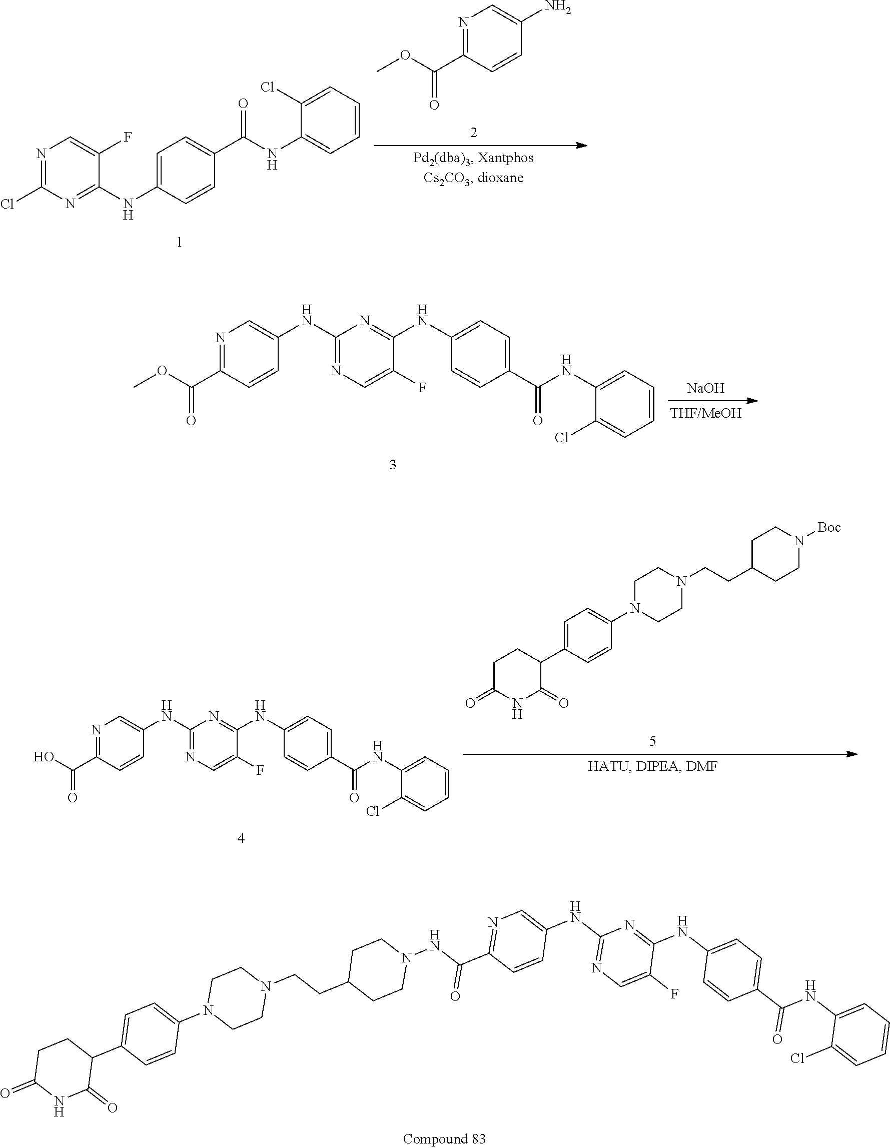

### Step 1. Synthesis of methyl 5-[[4-[4-[(2-chlorophenyl)carbamoyl]anilino]-5-fluoro-pyrimidin-2-yl]amino]pyridine-2-carboxylate (3)

A mixture of 4-[(2-chloro-5-fluoro-pyrimidin-4-yl)amino]-N-(2-chlorophenyl) benzamide (1 g, 2.65 mmol), methyl 5-aminopyridine-2-carboxylate (484.04 mg, 3.18 mmol), Pd₂(dba)₃ (242.77 mg, 265.11 μmol), Xantphos (306.80 mg, 530.22 μmol) and Cs₂CO₃ (2.59 g, 7.95 mmol) in dioxane (10 mL) was degassed and purged with N₂ for 3 times, and then the mixture was stirred at 100° C. for 16 hr under N₂ atmosphere. LCMS showed a peak (24%) with the desired mass. The reaction mixture was concentrated under reduced pressure. The residue was purified by flash silica gel chromatography (12 g SepaFlash® Silica Flash Column, Eluent of 20~75% EtOAc/Petroleum ether, gradient @ 60 mL/min) to afford crude product, which was purified by prep-HPLC (column: Phenomenex luna C18 150*40 mm*15 um; mobile phase: [water (TFA)-ACN]; gradient: 35%-65% B over 10 min) and lyophilized to afford methyl 5-[[4-[4-[(2-chlorophenyl)carbamoyl]anilino]-5-fluoro-pyrimidin-2-yl]amino]pyridine-2-carboxylate (350 mg, 568.08 mol, 21.43% yield, 80% purity) as a white solid. MS(M+H)⁺=493.1.

### Step 2. Synthesis of 5-[[4-[4-[(2-chlorophenyl]carbamoyl]anilino]-5-fluoro-pyrimidin-2-yl]amino]pyridine-2-carboxylic acid (4)

To a solution of methyl 5-[[4-[4-[(2-chlorophenyl) carbamoyl]anilino]-5-fluoro-pyrimidin-2-yl]amino]pyridine-2-carboxylate (350 mg, 710.10 μmol) in THF (4 mL) and MeOH (1 mL) was added NaOH (3 M, 1 mL). The mixture was stirred at 40° C. for 16 hr. LCMS showed a peak (88%) with the mass of starting material. Another portion of NaOH (3 M, 2 mL) was added and the mixture was stirred at 40° C. for 16 hr. LCMS showed a peak (95%) with the mass of desired mass. The reaction mixture was neutralized with 1 M HCl and the mixture was filtered. The filter cake was washed with MeOH (10 mL) and concentrated to afford 5-[[4-[4-[(2-chlorophenyl)carbamoyl]anilino]-5-fluoro-pyrimidin-2-yl]amino]pyridine-2-carboxylic acid (300 mg, 626.48 mol, 88.22% yield, 100% purity) as yellow solid. MS(M+H)⁺=479.1.

### Step 3. Synthesis of 5-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]pyridine-2-carboxamide (Compound 83)

To a solution of 5-[[4-[4-[(2-chlorophenyl)carbamoyl]anilino]-5-fluoro-pyrimidin-2-yl]amino]pyridine-2-carboxylic acid (150 mg, 313.24 μmol) and 3-[4-[4-[2-(1-amino-4-piperidyl)ethyl]piperazin-1-yl]phenyl]piperidine-2,6-dione (204.86 mg, 469.86 mol, HCl) in DMF (3 mL) were added HATU (142.92 mg, 375.89 μmol) and DIPEA (121.45 mg, 939.73 mol, 163.68 μL). The mixture was stirred at 20° C. for 16 hr. LCMS showed a peak (69%) with desired mass. The reaction mixture was diluted with water (10 mL) and filtered. The filter cake was purified by prep-HPLC (column: Phenomenex luna C18 150*25 mm*10 um; mobile phase: [water (TFA)-ACN]; gradient: 29%-59% B over 10 min) and lyophilized to afford 5-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]pyridine-2-carboxamide (44.3 mg, 50.46 μmol, 16.11% yield, 93.9% purity) as white solid. MS(M+H)⁺=860.4.

¹H NMR (400 MHz, CD₃OD-d₄) δ=8.90 (d, J=2.4 Hz, 1H), 8.32 (dd, J=2.5, 8.7 Hz, 1H), 8.15 (d, J=3.7 Hz, 1H), 8.08-8.00 (m, 3H), 8.00-7.93 (m, 2H), 7.83 (dd, J=1.4, 7.9 Hz, 1H), 7.59-7.52 (m, 1H), 7.45-7.37 (m, 1H), 7.34-7.26 (m, 1H), 7.22 (d, J=8.7 Hz, 2H), 7.05 (d, J=8.7 Hz, 2H), 3.97-3.79 (m, 3H), 3.70 (br d, J=11.4 Hz, 2H), 3.31-3.18 (m, 6H), 3.14-3.00 (m, 2H), 2.82-2.57 (m, 4H), 2.29-2.13 (m, 2H), 1.88-1.73 (m, 4H), 1.69-1.54 (m, 2H), 1.53-1.41 (m, 1H).

### Example 84. Synthesis of 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)-3-methoxybenzamide (Compound 84)

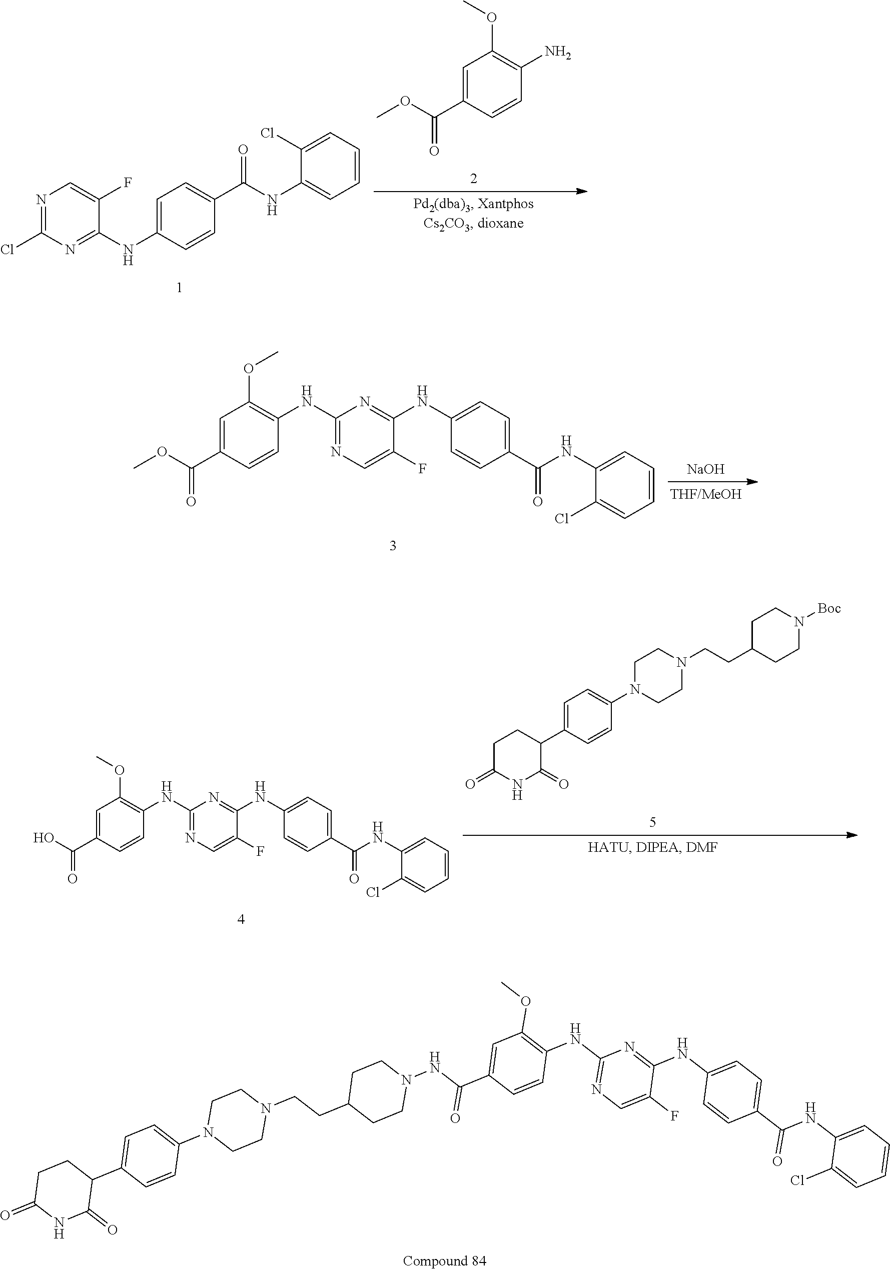

### Step 1. Synthesis of methyl 4-((4-((4-((2-chlorophenyl)carbamoyl)phenyl)amino)-5-fluoropyrimidin-2-yl)amino)-3-methoxybenzoate (3)

To a solution of 4-((2-chloro-5-fluoropyrimidin-4-yl)amino)-N-(2-chlorophenyl)benzamide (1 g, 2.65 mmol) and methyl methyl 4-amino-3-methoxybenzoate (480.35 mg, 2.65 mmol) in dioxane (10 mL) was added TsOH·H₂O (1.51 g, 7.95 mmol). The mixture was stirred at 100° C. for 16 hr. LCMS showed ~2% of 4-((2-chloro-5-fluoropyrimidin-4-yl)amino)-N-(2-chlorophenyl)benzamide remained and ~62% peak of desired mass. The mixture was treated with Na₂CO₃ solution to adjust pH=7 and the suspension was filtered. The filtrate was extracted with EtOAc (30 mL×5). The filter cake was washed with EtOAc (30 mL×4). The combined organic layers were dried over Na₂SO₄, filtered and concentrated in vacuum. The crude product was triturated with petroleum ether/EtOAc (2:1, 30 mL) at 25° C. for 30 min to afford methyl 4-((4-((4-((2-chlorophenyl)carbamoyl)phenyl)amino)-5-fluoropyrimidin-2-yl)amino)-3-methoxybenzoate (700 mg, 1.17 mmol, 44.01% yield, 87% purity) as a light yellow solid. MS(M+H)⁺=522.1.

### Step 2. Synthesis of 4-((4-((4-((2-chlorophenyl)carbamoyl)phenyl)amino)-5-fluoropyrimidin-2-yl) amino)-3-methoxybenzoic acid (4)

To a solution of methyl 4-((4-((4-((2-chlorophenyl)carbamoyl)phenyl)amino)-5-fluoropyrimidin-2-yl)amino)-3-methoxybenzoate (700 mg, 1.17 mmol) in THF (7 mL) and MeOH (2 mL) was added NaOH (3 M, 2 mL). The mixture was stirred at 40° C. for 24 hr. LCMS showed the starting material was consumed completely and one main peak with desired mass. The mixture was neutralized to pH=7 with 2N HCl solution and filtered. The filter cake was collected, washed with MeOH (30 mL) and dried to afford 4-((4-((4-((2-chlorophenyl)carbamoyl)phenyl)amino)-5-fluoropyrimidin-2-yl)amino)-3-methoxybenzoic acid (500 mg, 984.45 μmol, 84.37% yield) as a white solid. MS(M+H)⁺=508.2.

### Step 3. Synthesis of 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl-3-methoxybenzamide (Compound 84)

To a solution of 4-((4-((4-((2-chlorophenyl)carbamoyl)phenyl)amino)-5-fluoropyrimidin-2-yl)amino)-3-methoxybenzoic acid (200 mg, 393.78 μmol) in DMF (4 mL) was added HATU (224.59 mg, 590.67 μmol) and DIPEA (254.46 mg, 1.97 mmol, 342.94 μL) and 3-(4-(4-(2-(1-aminopiperidin-4-yl)ethyl)piperazin-1-yl)phenyl)piperidine-2,6-dione (206.02 mg, 472.53 μmol, HCl salt). The mixture was stirred at 25° C. for 2 hr. LCMS showed 3-(4-(4-(2-(1-aminopiperidin-4-yl)ethyl)piperazin-1-yl)phenyl)piperidine-2,6-dione was consumed completely and ~32% of desired compound was detected. The reaction mixture was poured into water (20 mL) and filtered. The filter cake was collected and purified by prep-HPLC (column: Phenomenex luna C18 150×40 mm×15 μm; mobile phase: [water(TFA)-ACN]; gradient: 25%-55% B over 10 min) and lyophilized to afford the product, which was further purified by prep-HPLC (column: Waters Xbridge C18 150×50 mm×10 μm; mobile phase: [water(NH₄HCO₃)-ACN]; gradient: 34%-64% B over 10 min) and lyophilized to afford 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)phenyl)piperazin-1-yl)ethyl)piperidin-1-yl)-3-methoxybenzamide (12.4 mg, 13.44 mol, 3.41% yield, 96.4% purity) as a white solid. MS(M+H)⁺=889.3.

¹H NMR (400 MHz, DMSO-d₆) δ=10.76 (br s, 1H), 9.92 (s, 1H), 9.76 (br s, 1H), 9.22 (s, 1H), 8.23 (d, J=3.5 Hz, 1H), 8.19 (d, J=8.4 Hz, 1H), 8.03 (s, 1H), 8.00-7.91 (m, 4H), 7.63 (dd, J=1.4, 7.9 Hz, 1H), 7.56 (dd, J=1.3, 7.9 Hz, 1H), 7.45-7.34 (m, 3H), 7.32-7.25 (m, 1H), 7.05 (d, J=8.6 Hz, 2H), 6.89 (br d, J=8.6 Hz, 2H), 3.91 (s, 3H), 3.72 (dd, J=5.0, 10.8 Hz, 1H), 3.49-3.41 (m, 4H), 3.14-3.07 (m, 4H), 3.02-2.94 (m, 2H), 2.77-2.68 (m, 2H), 2.63-2.58 (m, 1H), 2.46-2.42 (m, 1H), 2.37-2.32 (m, 2H), 2.18-2.07 (m, 1H), 2.05-1.96 (m, 1H), 1.76-1.63 (m, 2H), 1.48-1.37 (m, 2H), 1.35-1.23 (m, 3H).

### Example 85. Synthesis of N-(2-chlorophenyl)-4-((2-((4-((4-(2-(4-(4-((2,6-dioxopiperidin-3-yl)amino)-3-methylphenyl)piperazin-1-yl)ethyl)piperidin-1-yl)carbamoyl)phenyl)amino)-5-fluoropyrimidin-4-yl)amino)benzamide (Compound 85)

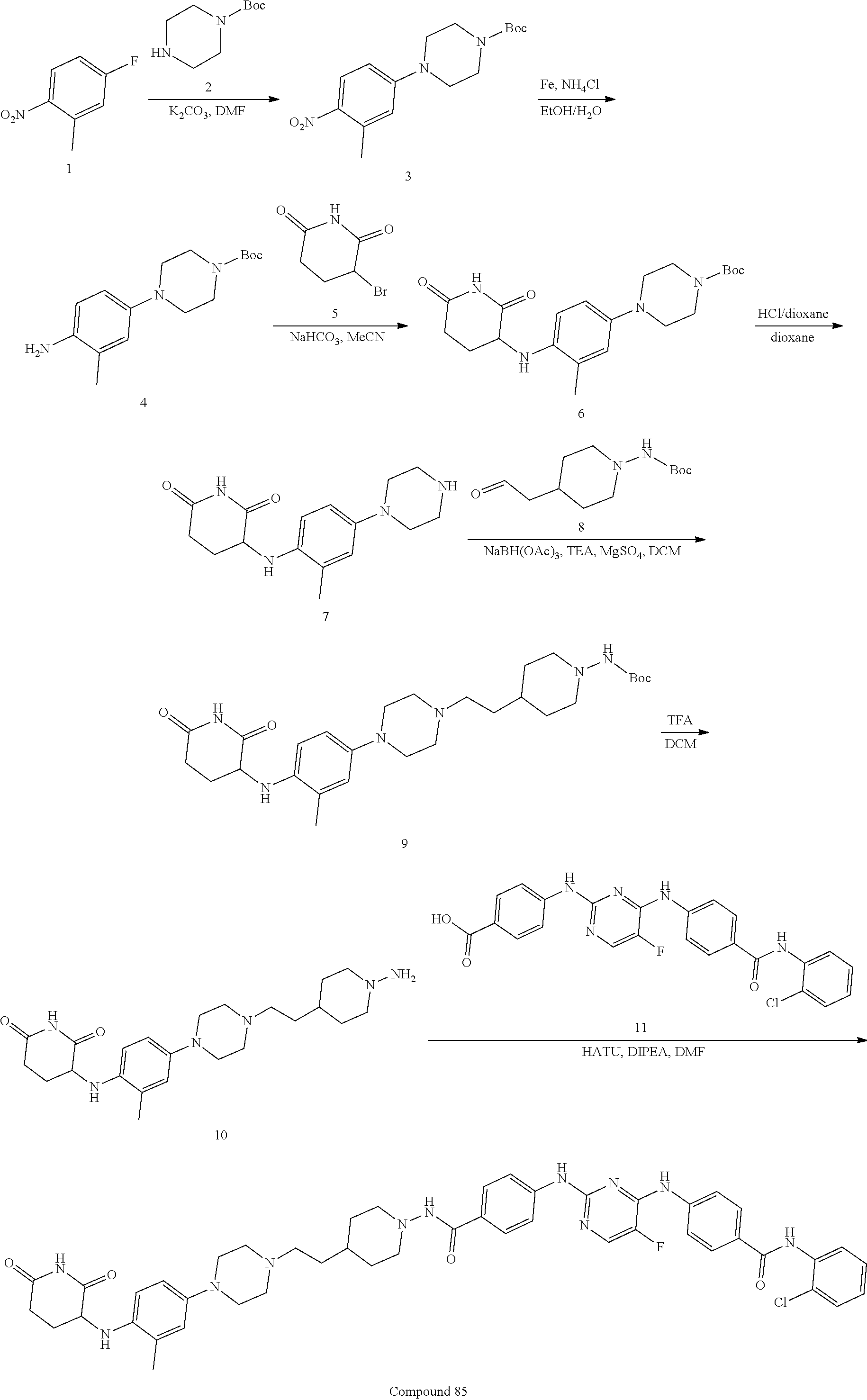

### Step 1. Synthesis of tert-butyl 4-(3-methyl-4-nitrophenyl)piperazine-1-carboxylate

To a solution of 4-fluoro-2-methyl-1-nitrobenzene (5 g, 32.23 mmol, 3.93 mL) and tert-butyl piperazine-1-carboxylate (9.00 g, 48.35 mmol) in DMF (60 mL) was added K₂CO₃ (13.36 g, 96.70 mmol) at 20′° C. The mixture was stirred at 60° C. for 16 h. LCMS showed 4-fluoro-2-methyl-1-nitrobenzene was consumed completely and 64% peak with desired mass was detected. The reaction mixture was diluted with H₂O (80 ml) and extracted with EtOAc (40 mL×3). The combined organic layers were washed with brine (40 mL×3), dried over Na₂SO₄, filtered and concentrated in vacuum. The residue was purified by flash silica gel chromatography (ISCO®; 40 g SepaFlash® Silica Flash Column, Eluent of 18-50% EtOAc/Petroleum ether gradient @ 100 mL/min) to afford the crude product, which was further purified by trituration with MTBE (15 mL) to afford tert-butyl 4-(3-methyl-4-nitrophenyl)piperazine-1-carboxylate (4.25 g, 13.22 mmol, 41.03% yield) as a yellow solid. MS(M+H)⁺=322.2.

### Step 2. Synthesis of tert-butyl 4-(4-amino-3-methylphenyl)piperazine-1-carboxylate

To a solution of tert-butyl 4-(3-methyl-4-nitrophenyl)piperazine-1-carboxylate (5 g, 15.56 mmol) in EtOH (60 mL) and H₂O (20 mL) were added Fe (3.48 g, 62.23 mmol) and NH₄Cl (4.16 g, 77.79 mmol) at 20° C. The mixture was stirred at 80° C. for 2 h. LCMS showed the starting material was consumed completely and 98% peak with desired mass (Rₜ=0.853 min) was detected. The reaction mixture was filtered and the filtrate was concentrated in vacuum to give a residue. The residue was diluted with H₂O (150 mL), adjusted to pH=10 with Na₂CO₃ and extracted with EtOAc (60 mL×3). The combined organic layers were dried over Na₂SO₄ and concentrated in vacuum to afford tert-butyl 4-(4-amino-3-methylphenyl)piperazine-1-carboxylate (4.1 g, 13.93 mmol, 89.53% yield, 99% purity) as a white solid. MS(M+H)⁺=292.2.

### Step 3. Synthesis of tert-butyl 4-(4-((2,6-dioxopiperidin-3-yl)amino)-3-methylphenyl)piperazine-1-carboxylate (6)

To a solution of tert-butyl 4-(4-amino-3-methylphenyl)piperazine-1-carboxylate (1 g, 3.43 mmol) and 3-bromopiperidine-2,6-dione (988.43 mg, 5.15 mmol) in MeCN (4 mL) was added NaHCO₃ (864.90 mg, 10.30 mmol) at 20° C. The mixture was stirred at 80° C. for 16 h. LCMS showed tert-butyl 4-(4-amino-3-methylphenyl)piperazine-1-carboxylate was consumed completely and 62% peak with desired mass (Rₜ=0.452 min) was detected. The mixture was filtered and the filter cake was washed with a mixed solution of petroleum ether and MeOH (50 mL, 1:1). The filtrate was concentrated in vacuum to give a residue, which was purified by flash silica gel chromatography (ISCO®; 12 g SepaFlash® Silica Flash Column, Eluent of 40-100% EtOAc/Petroleum ether gradient @ 80 mL/min) to afford tert-butyl 4-(4-((2,6-dioxopiperidin-3-yl)amino)-3-methylphenyl)piperazine-1-carboxylate (550 mg, 1.37 mmol, 39.82% yield) as a gray solid. MS(M+H)⁺=403.2.

### Step 4. Synthesis of 3-((2-methyl-4-(piperazin-1-yl)phenyl)amino)piperidine-2,6-dione (7)

To a solution of tert-butyl 4-(4-((2,6-dioxopiperidin-3-yl)amino)-3-methylphenyl)piperazine-1-carboxylate (550 mg, 1.37 mmol) in dioxane (3 mL) was added HCl/dioxane (4 M, 10 mL) at 20° C. The mixture was stirred at 20° C. for 3 h. LCMS showed the starting material was consumed completely and 94% peak with desired mass was detected. The reaction mixture was concentrated in vacuum to afford 3-((2-methyl-4-(piperazin-1-yl)phenyl)amino)piperidine-2,6-dione (590 mg, crude, HCl) as a blue solid. MS(M+H)⁺=303.2.

### Step 5. Synthesis of tert-butyl (4-(2-(4-(4-((2,6-dioxopiperidin-3-yl)amino)-3-methylphenyl)piperazin-1-yl)ethyl)piperidin-1-yl)carbamate (9)

To a solution of 3-((2-methyl-4-(piperazin-1-yl)phenyl)amino)piperidine-2,6-dione (300 mg, 885.40 μmol, HCl) and tert-butyl (4-(2-oxoethyl)piperidin-1-yl)carbamate (321.82 mg, 1.33 mmol) in DCM (5 mL) were added TEA (268.78 mg, 2.66 mmol, 369.71 μL) and MgSO₄ (213.15 mg, 1.77 mmol). After stirring 10 min, NaBH(OAc)₃ (562.95 mg, 2.66 mmol) was slowly added and the resulting mixture was stirred at 25° C. for 16 h. LCMS showed 3-((2-methyl-4-(piperazin-1-yl)phenyl)amino)piperidine-2,6-dione was consumed completely and 68% peak with desired mass. The reaction mixture was diluted with H₂O (30 ml) and extracted with DCM (12 mL×3). The combined organic layers were washed with NaHCO₃ (10 mL×2), dried over Na₂SO₄, filtered and concentrated in vacuum. The residue was purified by flash silica gel chromatography (ISCO®; 4 g SepaFlash® Silica Flash Column, Eluent of 80-100% EtOAc/Petroleum ether to 0~20% MeOH/EtOAc gradient @ 100 mL/min) to afford the crude product, which was further purified by prep-HPLC (column: Phenomenex luna C18 150*40 mm*15 um; mobile phase: [water(TFA)-ACN]; gradient: 10%-40% B over 10 min) and lyophilized to afford tert-butyl (4-(2-(4-(4-((2,6-dioxopiperidin-3-yl)amino)-3-methylphenyl)piperazin-1-yl)ethyl)piperidin-1-yl) carbamate (205 mg, 318.96 μmol, 36.02% yield, TFA) as a gray solid. MS(M+H)⁺=529.5.

### Step 6. Synthesis of 3-((4-(4-(2-(1-aminopiperidin-4-yl)ethyl)piperazin-1-yl)-2-methylphenyl) amino)piperidine-2,6-dione (10)

To a solution of tert-butyl (4-(2-(4-(4-((2,6-dioxopiperidin-3-yl)amino)-3-methylphenyl)piperazin-1-yl)ethyl)piperidin-1-yl)carbamate (180 mg, 280.06 μmol, TFA) in DCM (2 mL) was added TFA (159.67 mg, 1.40 mmol, 104.02 μL) at 20° C. The mixture was stirred at 20° C. for 2 hr. LCMS showed the starting material was consumed completely and one main peak (96%) with desired mass was detected. The reaction mixture was concentrated in vacuum to afford 3-((4-(4-(2-(1-aminopiperidin-4-yl)ethyl)piperazin-1-yl)-2-methylphenyl)amino)piperidine-2,6-dione (155 mg, crude, TFA) as a brown oil. MS(M+H)⁺=429.3.

### Step 7. Synthesis of N-(2-chlorophenyl)-4-((2-((4-((4-(2-(4-(4-((2,6-dioxopiperidin-3-yl)amino)-3-methylphenyl)piperazin-1-ylethyl)piperidin-1-yl)carbamoyl)phenyl)amino)-5-fluoropyrimidin-4-yl)amino)benzamide (Compound 85)

To a solution of 4-((4-((4-((2-chlorophenyl)carbamoyl)phenyl)amino)-5-fluoropyrimidin-2-yl)amino)benzoic acid (136.51 mg, 285.67 μmol) in DMF (2 mL) were added HATU (108.62 mg, 285.67 μmol) and DIPEA (110.76 mg, 857.00 μmol, 149.27 μL) at 20° C. After stirring for 10 min, a solution of 3-((4-(4-(2-(1-aminopiperidin-4-yl)ethyl)piperazin-1-yl)-2-methylphenyl)amino)piperidine-2,6-dione (155 mg, 285.67 mol, TFA) in DMF (2 mL) was added and the resulting mixture was stirred at 20° C. for 1 hr. LCMS showed 3-((4-(4-(2-(1-aminopiperidin-4-yl)ethyl)piperazin-1-yl)-2-methylphenyl)amino)piperidine-2,6-dione was consumed completely and the desired mass (Rₜ=0.621 min) was detected. The mixture was diluted with water (20 mL) and then filtered. The filter cake was washed with water (10 mL) and EtOAc (5 mL). The crude product was purified by prep-HPLC (column: Phenomenex luna C18 150*40 mm*15 um; mobile phase: [water(TFA)-ACN]; gradient: 25%-55% B over 10 min) and lyophilized to afford N-(2-chlorophenyl)-4-((2-((4-((4-(2-(4-(4-((2,6-dioxopiperidin-3-yl)amino)-3-methylphenyl)piperazin-1-yl)ethyl)piperidin-1-yl)carbamoyl)phenyl)amino)-5-fluoropyrimidin-4-yl)amino)benzamide (71.9 mg, 52.59 μmol, 18.41% yield, 90% purity, 3TFA) as a gray solid. MS(M+H)⁺=888.3

¹H NMR (400 MHz, DMSO-d₆) δ=10.85 (s, 1H), 9.94 (s, 1H), 9.78 (s, 1H), 9.76-9.70 (m, 1H), 9.68 (s, 1H), 9.54-9.37 (m, 1H), 8.26 (d, J=3.5 Hz, 1H), 8.06-7.97 (m, 4H), 7.82-7.77 (m, 2H), 7.74-7.70 (m, 2H), 7.65 (dd, J=1.5, 7.9 Hz, 1H), 7.57 (dd, J=1.3, 7.9 Hz, 1H), 7.40 (dt, J=1.4, 7.7 Hz, 1H), 7.34-7.27 (m, 1H), 6.80-6.76 (m, 1H), 6.71 (dd, J=2.4, 8.6 Hz, 1H), 6.64-6.58 (m, 1H), 4.29 (br dd, J=4.8, 12.0 Hz, 1H), 3.57-3.56 (m, 2H), 3.29-3.05 (m, 8H), 2.89-2.81 (m, 4H), 2.80-2.72 (m, 1H), 2.61-2.53 (m, 1H), 2.20-2.10 (m, 4H), 2.00-1.90 (m, 1H), 1.79-1.72 (m, 2H), 1.68-1.61 (m, 2H), 1.46-1.34 (m, 3H).

### Example 86. Synthesis of N-(2-chlorophenyl)-4-((2-((4-(((1r,4r)-4-(2-(4-(4-((2,6-dioxopiperidin-3-yl)amino)-2-fluorophenyl)piperazin-1-yl)ethyl)cyclohexyl)carbamoyl)phenyl)amino)-5-fluoropyrimidin-4-yl)amino)benzamide (Compound 86)

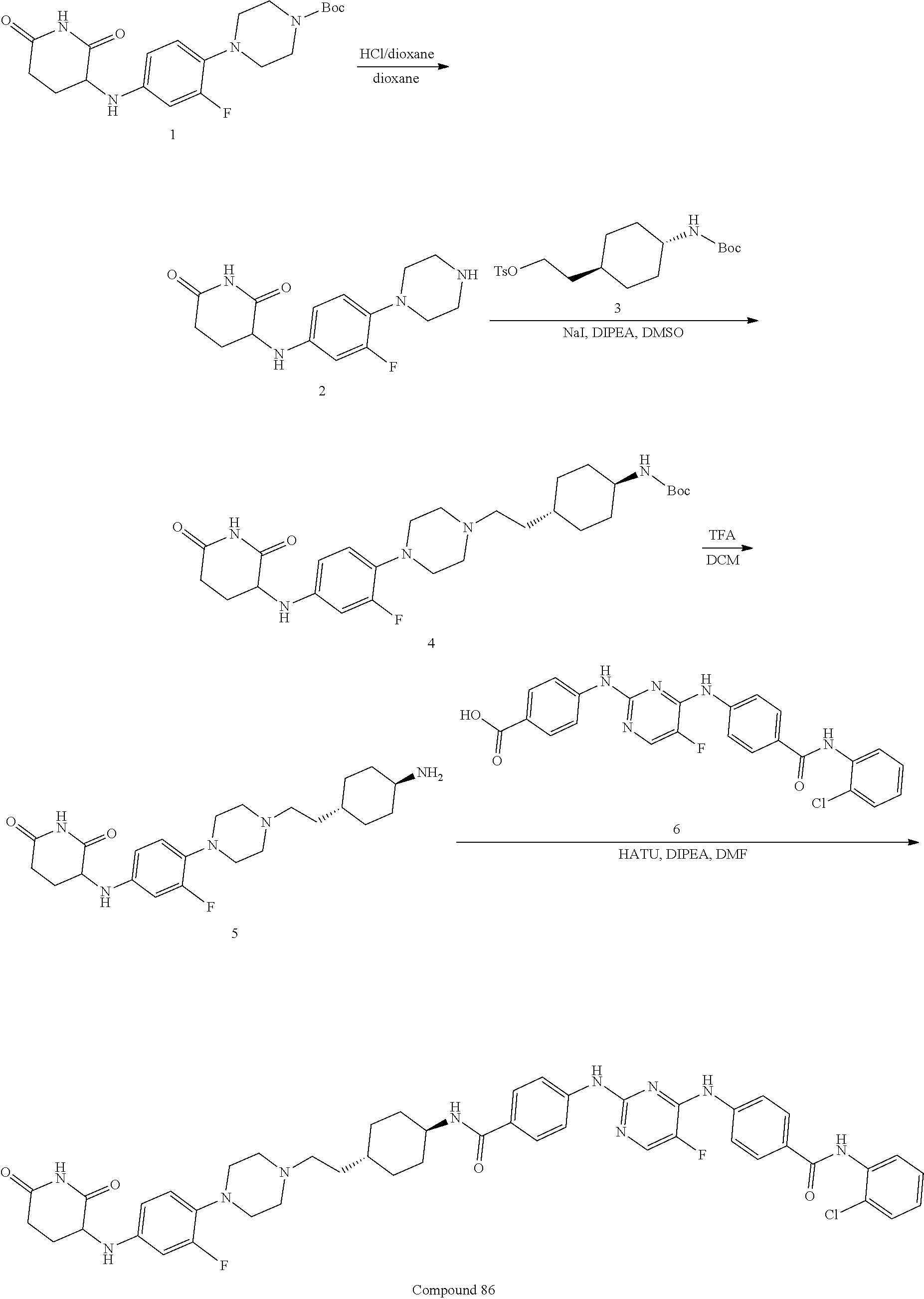

### Step 1. Synthesis of 3-((3-fluoro-4-(piperazin-1-yl)phenyl)amino)piperidine-2,6-dione (2)

To a solution of tert-butyl 4-(4-((2,6-dioxopiperidin-3-yl)amino)-2-fluorophenyl)piperazine-1-carboxylate (2.7 g, 6.64 mmol) in dioxane (10 mL) was added HCl/dioxane (4 M, 20 mL) at 20° C. The mixture was stirred at 20° C. for 1 h. LCMS showed 97% peak with desired mass was detected. The reaction mixture was concentrated in vacuum to afford 3-((3-fluoro-4-(piperazin-1-yl)phenyl)amino)piperidine-2,6-dione (3.1 g, crude, HCl) as a gray solid. MS(M+H)⁺=307.1.

### Step 2. Synthesis of tert-butyl ((1r,4r)-4-(2-(4-(4-((2,6-dioxopiperidin-3-yl)amino)-2-fluorophenyl)piperazin-1-yl)ethyl)cyclohexyl)carbamate (4)

To a solution of 3-((3-fluoro-4-(piperazin-1-yl)phenyl)amino)piperidine-2,6-dione (300 mg, 875.16 μmol, HCl) and 2-((1r,4r)-4-((tert-butoxycarbonyl)amino)cyclohexyl)ethyl 4-methylbenzenesulfonate (417.48 mg, 1.05 mmol) in DMSO (5 mL) were added NaI (13.12 mg, 87.52 μmol) and DIPEA (339.32 mg, 2.63 mmol, 457.30 μL) at 20° C. The mixture was stirred at 80° C. for 16 h. LCMS showed 2-((1r,4r)-4-((tert-butoxycarbonyl)amino)cyclohexyl)ethyl 4-methylbenzenesulfonate was consumed completely, 2% of 3-((3-fluoro-4-(piperazin-1-yl)phenyl)amino)piperidine-2,6-dione remained and desired mass (Rₜ=0.552 min) was detected. The reaction mixture was poured into water (10 mL) and extracted with EtOAc (6 mL×4). The combined organic layers were dried over Na₂SO₄ and concentrated in vacuum to give a residue. The residue was purified by flash silica gel chromatography (ISCO®; 12 g SepaFlash® Silica Flash Column, Eluent of 80~100% EtOAc/Petroleum ether to 0-10% MeOH/EtOAc gradient @ 80 mL/min) to afford the impure product, which was further purified by prep-HPLC (column: Phenomenex luna C18 150*40 mm*15 um; mobile phase: [water(TFA)-ACN]; gradient: 25%-55% B over 10 min) and lyophilized to afford tert-butyl ((1r,4r)-4-(2-(4-(4-((2,6-dioxopiperidin-3-yl)amino)-2-fluorophenyl)piperazin-1-yl)eth yl)cyclohexyl)carbamate (125 mg, 180.04 mol, 20.57% yield, 93% purity, TFA) as a light yellow solid. MS(M+H)⁺=532.4.

### Step 3. Synthesis of 3-((4-(4-(2-((1r,4r)-4-aminocyclohexyl)ethyl)piperazin-1-yl)-3-fluorophenyl) amino)piperidine-2,6-dione (5)

To a solution of tert-butyl ((1r,4r)-4-(2-(4-(4-((2,6-dioxopiperidin-3-yl)amino)-2-fluorophenyl)piperazin-1-yl)eth yl)cyclohexyl)carbamate (100 mg, 154.87 mol, TFA) in DCM (2 mL) was added TFA (88.30 mg, 774.37 μmol, 57.52 μL) at 20° C. The mixture was stirred at 20° C. for 1 h. LCMS showed the starting material was consumed completely and desired mass was detected. The reaction mixture was concentrated in vacuum to afford 3-((4-(4-(2-((1r,4r)-4-aminocyclohexyl)ethyl)piperazin-1-yl)-3-fluorophenyl)amino)piperidine-2,6-dione (85 mg, crude, TFA) as a brown oil. MS(M+H)⁺=432.2.

### Step 4. Synthesis of N-(2-chlorophenyl)-4-((2-((4-(((1r,4r)-4-(2-(4-(4-((2,6-dioxopiperidin-3-yl)amino)-2-fluorophenyl)piperazin-1-yl)ethyl)cyclohexyl)carbamoyl)phenyl)amino)-5-fluoropyrimidin-4-yl)amino)benzamide (Compound 86)

To a solution of 4-((4-((4-((2-chlorophenyl)carbamoyl)phenyl)amino)-5-fluoropyrimidin-2-yl)amino)benzoic acid (74.45 mg, 155.80 μmol) in DMF (0.5 mL) were added HATU (59.24 mg, 155.80 μmol) and DIPEA (60.41 mg, 467.40 μmol, 81.41 μL) at 20° C. After stirring for 10 min, a solution of 3-((4-(4-(2-((1r,4r)-4-aminocyclohexyl)ethyl)piperazin-1-yl)-3-fluorophenyl)amino)piperidine-2,6-dione (85 mg, 155.80 μmol, TFA) in DMF (0.5 mL) was added and the resulting mixture was stirred at 20° C. for 1 h. LCMS showed the starting material remained and 16% peak with desired mass was detected. Another portion of DIPEA (148.40 mg, 1.15 mmol, 0.2 mL) was added and the mixture was stirred at 20° C. for 1 hr. LCMS showed 3-((4-(4-(2-((1r,4r)-4-aminocyclohexyl)ethyl)piperazin-1-yl)-3-fluorophenyl)amino)piperidine-2,6-dione was consumed completely and desired mass was detected. The mixture was diluted with water (20 mL) and then filtered. The filter cake was washed with water (10 mL) and EtOAc (5 mL). The filter cake was collected and purified by prep-HPLC (column: Phenomenex luna C18 150*40 mm*15 um; mobile phase: [water(TFA)-ACN]; gradient: 25%-55% B over 10 min). The fractions were lyophilized to afford N-(2-chlorophenyl)-4-((2-((4-(((1r,4r)-4-(2-(4-(4-((2,6-dioxopiperidin-3-yl)amino)-2-fluorophenyl)piperazin-1-yl)ethyl)cyclohexyl)carbamoyl)phenyl)amino)-5-fluoropyrimidin-4-yl) amino)benzamide (55.2 mg, 45.37 mol, 29.12% yield, 92% purity, 2TFA) as a white solid. MS(M+H)⁺=891.3.

¹H NMR (400 MHz, DMSO-d₆) δ=10.79 (s, 1H), 9.93 (s, 1H), 9.76 (s, 1H), 9.61 (s, 1H), 9.43-9.29 (m, 1H), 8.25 (d, J=3.5 Hz, 1H), 8.05-7.96 (m, 5H), 7.79-7.74 (m, 4H), 7.65 (dd, J=1.4, 8.0 Hz, 1H), 7.57 (dd, J=1.3, 7.9 Hz, 1H), 7.40 (dt, J=1.4, 7.7 Hz, 1H), 7.34-7.27 (m, 1H), 6.89 (t, J=9.4 Hz, 1H), 6.55 (dd, J=2.3, 15.1 Hz, 1H), 6.46 (dd, J=1.6, 9.3 Hz, 1H), 4.28 (dd, J=4.7, 11.6 Hz, 1H), 3.77-3.67 (m, 3H), 3.35-3.25 (m, 2H), 3.22-3.12 (m, 4H), 3.00-2.90 (m, 2H), 2.79-2.66 (m, 1H), 2.63-2.53 (m, 1H), 2.12-2.03 (m, 1H), 1.92-1.81 (m, 3H), 1.80-1.73 (m, 2H), 1.65-1.52 (m, 2H), 1.40-1.20 (m, 3H), 1.14-0.99 (m, 2H).

### Example 87. Synthesis of (S)-5-(4-(2-(1-(4-((4-((4-((2-chlorophenyl)carbamoyl)phenyl)amino)-5-fluoropyrimidin-2-yl)amino)benzamido)piperidin-4-yl)ethyl)piperazin-1-yl)-N-(2,6-dioxopiperidin-3-yl)picolinamide (Compound 87)

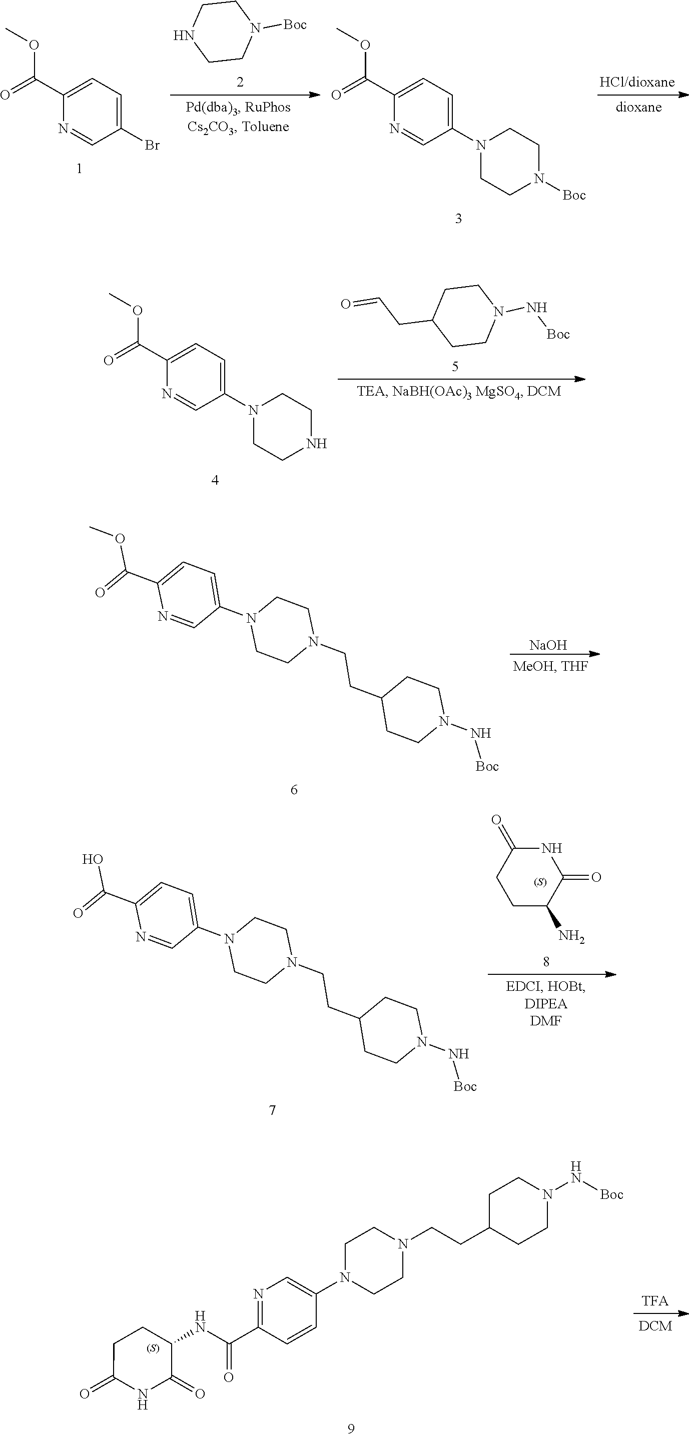

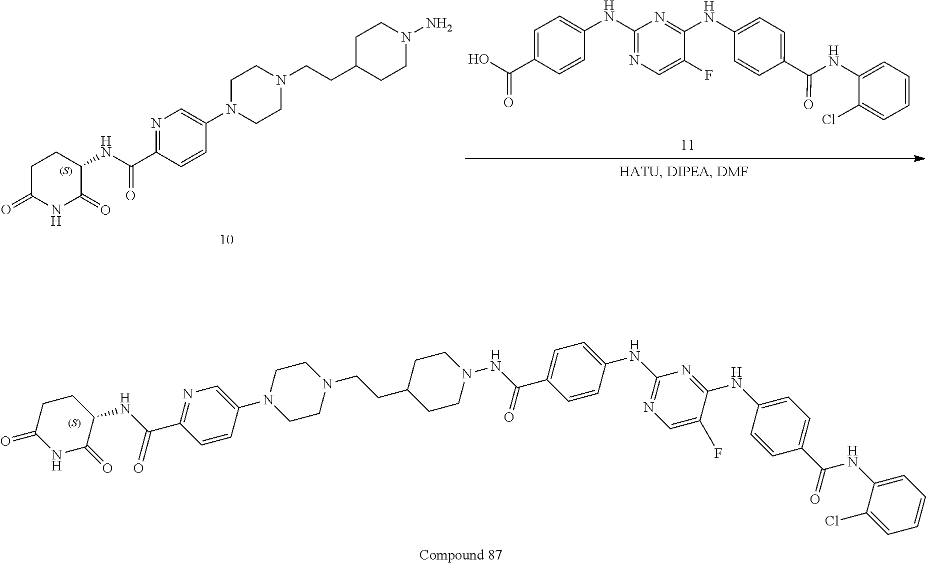

### Step 1. Synthesis of tert-butyl 4-(6-(methoxycarbonyl)pyridin-3-yl)piperazine-1-carboxylate (3)

To a solution of methyl 5-bromopicolinate (2 g, 9.26 mmol) and tert-butyl piperazine-1-carboxylate (1.72 g, 9.26 mmol) in toluene (40 mL) was added RuPhos (432.01 mg, 925.79 μmol), Pd₂(dba)₃ (423.88 mg, 462.89 μmol) and Cs₂CO₃ (9.05 g, 27.77 mmol) under N₂ atmosphere. The mixture was stirred at 100° C. for 16 hr under N₂ atmosphere. LCMS showed one main peak with desired mass was detected. The reaction mixture was filtered and concentrated under reduced pressure. The residue was purified by flash silica gel chromatography (ISCO®; 40 g SepaFlash® Silica Flash Column, Eluent of 70-80% EtOAc/Petroleum ether, gradient @ 100 mL/min) to afford tert-butyl 4-(6-(methoxycarbonyl)pyridin-3-yl)piperazine-1-carboxylate (2.7 g, 8.40 mmol, 90.75% yield) as a yellow solid.

¹H NMR (400 MHz, CDCl₃) δ=8.34 (d, J=2.9 Hz, 1H), 8.01 (d, J=8.8 Hz, 1H), 7.15 (dd, J=3.1, 8.8 Hz, 1H), 3.96 (s, 3H), 3.64-3.58 (m, 4H), 3.38-3.31 (m, 4H), 1.48 (s, 9H).

### Step 2. Synthesis of methyl 5-(piperazin-1-yl)picolinate (4)

To a solution of tert-butyl 4-(6-(methoxycarbonyl)pyridin-3-yl)piperazine-1-carboxylate (2.7 g, 8.40 mmol) in dioxane (30 mL) was added HCl/dioxane (4 M, 30 mL). The mixture was stirred at 25° C. for 1 hr. LCMS showed the starting material was consumed completely and one main peak with desired mass. The reaction mixture was concentrated under reduced pressure to afford methyl 5-(piperazin-1-yl)picolinate (3 g, crude, HCl salt) as a yellow solid. MS(M+H)⁺=222.1.

### Step 3. Synthesis of methyl 5-(4-(2-(1-((tert-butoxycarbonyl)amino)piperidin-4-yl)ethyl)piperazin-1-yl)picolinate

To a solution of methyl 5-(piperazin-1-yl)picolinate (500 mg, 1.94 mmol, HCl salt) and tert-butyl (4-(2-oxoethyl)piperidin-1-yl)carbamate (705.18 mg, 2.91 mmol) in DCM (10 mL) were added TEA (196.32 mg, 1.94 mmol, 270.04 μL) and MgSO₄ (233.53 mg, 1.94 mmol). After stirring at 20° C. for half an hour, NaBH(OAc)₃ (1.23 g, 5.82 mmol) was added and the resulting mixture was stirred at 25° C. for 16 hr. LCMS showed methyl 5-(piperazin-1-yl)picolinate was consumed completely and one main peak with desired mass. The residue was diluted with NaHCO₃ solution (20 mL) and extracted with DCM (20 mL×3). The combined organic layers were washed with water (20 mL), dried over Na₂SO₄, filtered and concentrated under reduced pressure. The residue was purified by flash silica gel chromatography (ISCO®; 20 g SepaFlash® Silica Flash Column, Eluent of 25-30% MeOH/EtOAc, gradient @ 80 mL/min) to afford methyl 5-(4-(2-(1-((tert-butoxycarbonyl)amino)piperidin-4-yl)ethyl)piperazin-1-yl)picolinate (640 mg, 1.43 mmol, 73.70% yield) as a light yellow solid. MS(M+H)⁺=448.3.

### Step 4. Synthesis of 5-(4-(2-(1-((tert-butoxycarbonyl)amino)piperidin-4-yl)ethyl)piperazin-1-yl)picolinic acid (7)

To a solution of methyl 5-(4-(2-(1-((tert-butoxycarbonyl)amino)piperidin-4-yl)ethyl)piperazin-1-yl)picolinate (640 mg, 1.43 mmol) in MeOH (4 mL) and THF (4 mL) was added NaOH (3 M, 953.30 μL). The mixture was stirred at 25° C. for 1 hr. LCMS showed the starting material was consumed completely and one main peak with desired mass. The reaction mixture was concentrated under reduced pressure to remove solvent. The residue was diluted with MeCN (20 mL) and added HCl (12 M) to adjust pH=6 and concentrated under reduced pressure to afford 5-(4-(2-(1-((tert-butoxycarbonyl)amino)piperidin-4-yl)ethyl)piperazin-1-yl)picolinic acid (750 mg, crude) as a white solid. MS(M+H)⁺=434.3.

### Step 5. Synthesis of tert-butyl (S)-(4-(2-(4-(6-((2,6-dioxopiperidin-3-yl)carbamoyl)pyridin-3-yl)piperazin-1-yl)ethyl) piperidin-1-yl)carbamate (9)

To a solution of 5-(4-(2-(1-((tert-butoxycarbonyl)amino)piperidin-4-yl)ethyl)piperazin-1-yl)picolinic acid (750 mg, 1.73 mmol) and (S)-3-aminopiperidine-2,6-dione (221.65 mg, 1.73 mmol) in DMF (10 mL) were added EDCI (497.44 mg, 2.59 mmol), HOBt (467.50 mg, 3.46 mmol) and DIPEA (670.73 mg, 5.19 mmol, 903.95 μL). The mixture was stirred at 25° C. for 16 hr. LCMS showed 5-(4-(2-(1-((tert-butoxycarbonyl)amino)piperidin-4-yl)ethyl)piperazin-1-yl)picolinic acid was consumed completely and ~23% peak of desired compound was detected. The reaction mixture was diluted with water (20 mL) and extracted with EtOAc (20 mL×3). The combined organic layers were washed with brine (20 mL×3), dried over Na₂SO₄, filtered and concentrated under reduced pressure to afford tert-butyl (S)-(4-(2-(4-(6-((2,6-dioxopiperidin-3-yl)carbamoyl)pyridin-3-yl)piperazin-1-yl)ethyl) piperidin-1-yl)carbamate (510 mg, 938.09 mol, 54.23% yield) as a white solid. MS(M+H)⁺=544.4.

### Step 6. Synthesis of (S)-5-(4-(2-(1-aminopiperidin-4-yl)ethyl)piperazin-1-yl)-N-(2,6-dioxopiperidin-3-yl)picolinamide (10)

To a solution of tert-butyl (S)-(4-(2-(4-(6-((2,6-dioxopiperidin-3-yl)carbamoyl)pyridin-3-yl)piperazin-1-yl)ethyl) piperidin-1-yl)carbamate (300 mg, 551.82 μmol) in DCM (6 mL) was added TFA (3.07 g, 26.93 mmol, 2 mL). The mixture was stirred at 25° C. for 1 hr. LCMS showed the starting material was consumed completely and one main peak with desired mass. The reaction mixture was concentrated under reduced pressure to afford (S)-5-(4-(2-(1-aminopiperidin-4-yl)ethyl)piperazin-1-yl)-N-(2,6-dioxopiperidin-3-yl)picolinamide (310 mg, crude, TFA salt) as a yellow oil. MS(M+H)⁺=444.3.

### Step 7. Synthesis of (S)-5-(4-(2-(1-(4-((4-((4-((2-chlorophenyl)carbamoyl)phenyl)amino)-5-fluoropyrimidin-2-yl)amino)benzamido)piperidin-4-yl)ethyl)piperazin-1-yl)-N-(2,6-dioxopiperidin-3-yl)picolinamide (Compound 87)

To a solution of 4-((4-((4-((2-chlorophenyl)carbamoyl)phenyl)amino)-5-fluoropyrimidin-2-yl)amino)benzoic acid (200 mg, 377.51 μmol) in DMF (5 mL) were added HATU (215.31 mg, 566.26 μmol), DIPEA (243.94 mg, 1.89 mmol, 328.77 μL) and
(S)-5-(4-(2-(1-aminopiperidin-4-yl)ethyl)piperazin-1-yl)-N-(2,6-dioxopiperidin-3-yl)picolinamide (273.63 mg, 490.76 mol, TFA salt). The mixture was stirred at 25° C. for 2 hr. LCMS showed
(S)-5-(4-(2-(1-aminopiperidin-4-yl)ethyl)piperazin-1-yl)-N-(2,6-dioxopiperidin-3-yl)picolinamide was consumed completely and one main peak with desired mass was detected. The reaction mixture was poured into water (20 mL) and filtered. The filter cake was collected to afford the crude product. The crude product was purified by prep-HPLC (column: Phenomenex luna C18 150×40 mm×15 μm; mobile phase: [water(TFA)-ACN]; gradient: 20%-50% B over 10 min) and lyophilized. The product was further purified by prep-HPLC (column: Waters Xbridge C18 150×50 mm×10 m; mobile phase: [water(NH₄HCO₃)-ACN]; gradient: 25%-55% B over 10 min) and lyophilized to afford 5-[4-[2-[1-[[4-[[4-[4-[(2-chlorophenyl)carbamoyl]anilino]-5-fluoro-pyrimidin-2-yl]amino]benzoyl]amino]-4-piperidyl]ethyl]piperazin-1-yl]-N-[(3S)-2,6-dioxo-3-piperidyl]pyridine-2-carboxamide (5.4 mg, 5.59 mol, 1.48% yield, 93.5% purity) and 120 mg crude product. The 120 mg of the crude product was further purified by prep-HPLC (column: Phenomenex luna C18 150×25 mm×10 μm; mobile phase: [water(TFA)-ACN]; gradient: 23%-53% B over 9 min) and lyophilized to afford 5-[4-[2-[1-[[4-[[4-[4-[(2-chlorophenyl)carbamoyl]anilino]-5-fluoro-pyrimidin-2-yl]amino]benzoyl]amino]-4-piperidyl]ethyl]piperazin-1-yl]-N-[(3S)-2,6-dioxo-3-piperidyl]pyridine-2-carboxamide (54.3 mg, 42.51 mol, 11.26% yield, 97.5% purity, 3TFA) as a white solid. MS(M+H)⁺=903.0.

¹H NMR (400 MHz, DMSO-d₆) δ=10.86 (s, 1H), 9.94 (s, 1H), 9.78 (s, 1H), 9.74-9.58 (m, 3H), 8.78 (d, J=8.3 Hz, 1H), 8.41 (d, J=2.6 Hz, 1H), 8.26 (d, J=3.5 Hz, 1H), 8.06-7.98 (m, 4H), 7.93 (d, J=8.6 Hz, 1H), 7.83-7.76 (m, 2H), 7.75-7.69 (m, 2H), 7.65 (dd, J=1.4, 8.0 Hz, 1H), 7.59-7.51 (m, 2H), 7.43-7.37 (m, 1H), 7.33-7.27 (m, 1H), 4.79-4.72 (m, 1H), 4.14-4.11 (m, 2H), 3.67-3.59 (m, 2H), 3.26-3.11 (m, 8H), 2.89-2.74 (m, 3H), 2.57-2.53 (m, 1H), 2.26-2.12 (m, 1H), 2.06-1.95 (m, 1H), 1.83-1.71 (m, 2H), 1.70-1.57 (m, 2H), 1.42-1.28 (m, 3H).

### Example 88. Synthesis of N-(2-chlorophenyl)-4-((2-((4-((4-(2-(4-(6-(2,6-dioxopiperidin-3-yl)pyridin-3-yl)piperazin-1-yl)ethyl)piperidin-1-yl)carbamoyl)phenyl)amino)-5-fluoropyrimidin-4-yl)amino)benzamide (Compound 88)

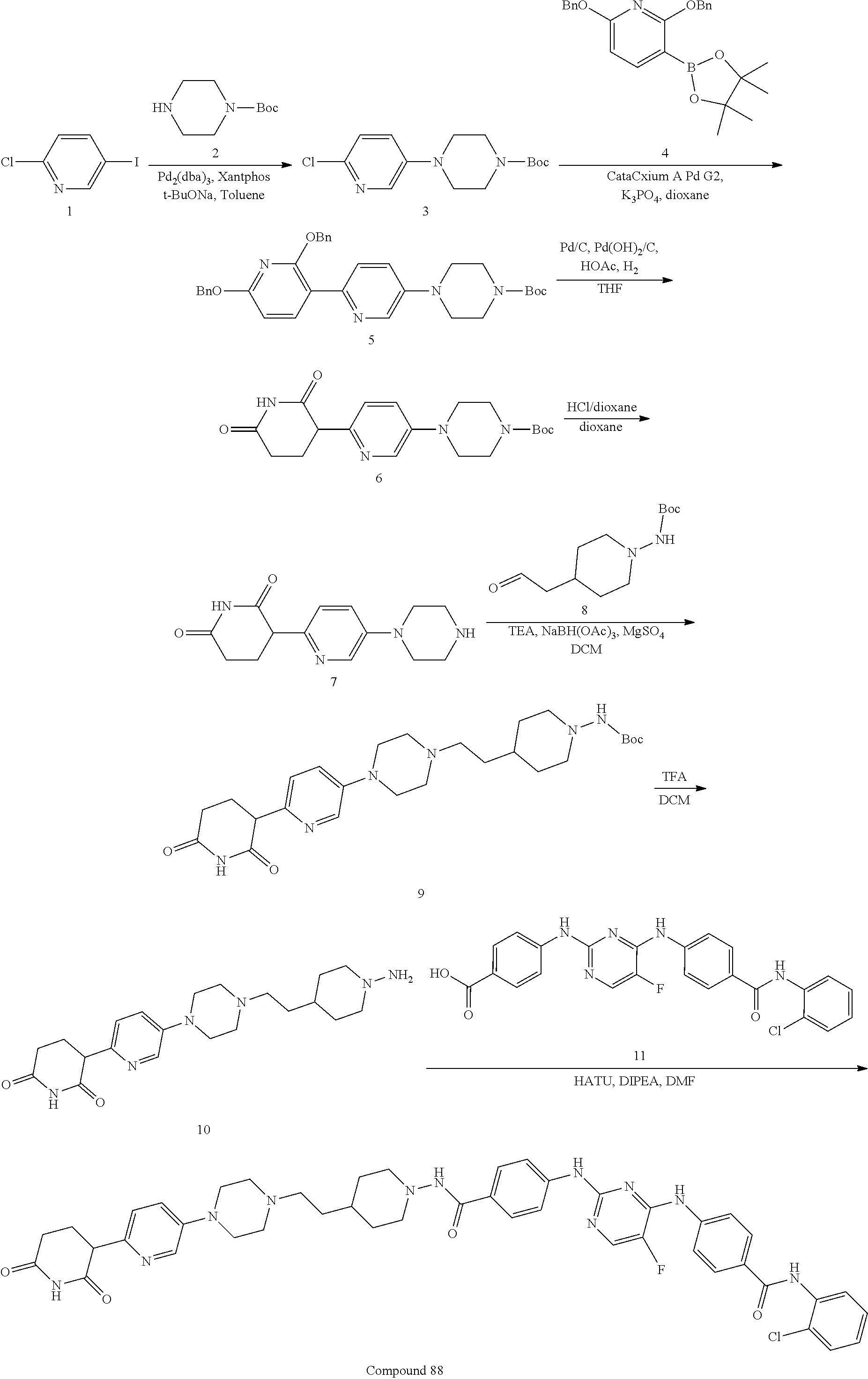

### Step 1. Synthesis of tert-butyl 4-(6-chloropyridin-3-yl)piperazine-1-carboxylate (3)

To a solution of 2-chloro-5-iodopyridine (5 g, 20.88 mmol) and tert-butyl piperazine-1-carboxylate (3.89 g, 20.88 mmol) in toluene (100 mL) were added t-BuONa (3.01 g, 31.32 mmol) and Pd₂(dba)₃ (956.10 mg, 1.04 mmol) and Xantphos (1.81 g, 3.13 mmol) under N₂ atmosphere. The mixture was stirred at 100° C. for 16 hr under N₂ atmosphere. LCMS showed one main peak with desired mass was detected. The reaction mixture was diluted with water (50 mL) and extracted with EtOAc (50 mL×3). The combined organic layers were washed with water (50 mL), dried over Na₂SO₄, filtered and concentrated under reduced pressure. The residue was purified by flash silica gel chromatography (ISCO®; 80 g SepaFlash® Silica Flash Column, Eluent of 25-35% EtOAc/Petroleum ether gradient @ 100 mL/min) to afford tert-butyl 4-(6-chloropyridin-3-yl)piperazine-1-carboxylate (3.9 g, 13.10 mmol, 62.72% yield) as a yellow solid.

¹H NMR (400 MHz, CDCl₃) δ=8.02 (t, J=1.8 Hz, 1H), 7.21-7.15 (m, 2H), 3.66-3.55 (m, 4H), 3.22-3.07 (m, 4H), 1.48 (s, 9H).

### Step 2. Synthesis of tert-butyl 4-(2′,6′-bis(benzyloxy)-[2,3′-bipyridin]-5-yl)piperazine-1-carboxylate (5)

To a solution of tert-butyl 4-(6-chloropyridin-3-yl)piperazine-1-carboxylate (2 g, 6.72 mmol) and 2,6-bis(benzyloxy)-3-(4,4,5,5-tetramethyl-1,3,2-dioxaborolan-2-yl)pyridine (2.80 g, 6.72 mmol,) in dioxane (40 mL) was added K₃PO₄ (1.5 M, 13.43 mL) and [2-(2-aminophenyl)phenyl]-chloro-palladium; bis(1-adamantyl)-butyl-phosphane (449.08 mg, 671.64 μmol) under N₂ atmosphere. The mixture was stirred at 80° C. for 16 hr under N₂ atmosphere. LCMS showed 13% peak of tert-butyl 4-(6-chloropyridin-3-yl)piperazine-1-carboxylate remained and ~34% peak with desired mass. The reaction mixture was diluted with water (30 mL) and extracted with EtOAc (30 mL×3). The combined organic layers were washed with brine (20 mL), dried over Na₂SO₄, filtered and concentrated under reduced pressure. The residue was purified by flash silica gel chromatography (ISCO®; 80 g SepaFlash® Silica Flash Column, Eluent of 10~20% EtOAc/Petroleum ether gradient @ 80 mL/min) to afford tert-butyl 4-(2′,6′-bis(benzyloxy)-[2,3′-bipyridin]-5-yl)piperazine-1-carboxylate (1.94 g, 3.51 mmol, 52.26% yield) as a yellow solid. MS(M+H)⁺=553.3.

### Step 3. Synthesis of tert-butyl 4-(6-(2,6-dioxopiperidin-3-yl)pyridin-3-yl)piperazine-1-carboxylate (6)

To a 30 mL hydrogenation bottle was cleaned and dried, with N₂ for 1 minute. To a mixture of Pd/C (20 mg, 10% purity) and Pd(OH)₂ (20 mg, 15% purity) in THF (5 mL) was added tert-butyl 4-(2′,6′-bis(benzyloxy)-[2,3′-bipyridin]-5-yl)piperazine-1-carboxylate (300 mg, 542.83 mol) and HOAc (4 mg, 66.61 μmol, 3.81 μL) in THF (5 mL) under N₂ atmosphere. The reaction system was replaced three times by argon and then three times by H₂. The mixture was stirred under H₂ (50 Psi) atmosphere at 25° C. for 32 hr. LCMS showed the starting material was consumed completely and one main peak with desired mass. The mixture was diluted with THF (20 mL), filtered and washed with THF (30 mL×3). The filtrate was concentrated under reduced pressure to remove solvent. The residue was purified by flash silica gel chromatography (ISCO®; 20 g SepaFlash® Silica Flash Column, Eluent of 5-10% MeOH/EtOAc gradient @ 80 mL/min) to afford tert-butyl 4-(6-(2,6-dioxopiperidin-3-yl)pyridin-3-yl)piperazine-1-carboxylate (110 mg, 293.78 μmol, 54.12% yield) as a yellow solid. MS(M+H)⁺=375.2.

### Step 4. Synthesis of 3-(5-(piperazin-1-yl)pyridin-2-yl)piperidine-2,6-dione (7)

To a solution of tert-butyl 4-(6-(2,6-dioxopiperidin-3-yl)pyridin-3-yl)piperazine-1-carboxylate (150 mg, 400.61 mol) in dioxane (1 mL) was added HCl/dioxane (2 M, 2 mL). The mixture was stirred at 25° C. for 1 hr. LCMS showed the starting material was consumed completely. The reaction mixture was concentrated under reduced pressure to afford 3-(5-(piperazin-1-yl)pyridin-2-yl)piperidine-2,6-dione (125 mg, crude, HCl salt) as a yellow solid. MS(M+H)⁺=275.1.

### Step 5. Synthesis of tert-butyl (4-(2-(4-(6-(2,6-dioxopiperidin-3-yl)pyridin-3-yl)piperazin-1-yl)ethyl)piperidin-1-yl)carbamate (9)

To a solution of 3-(5-(piperazin-1-yl)pyridin-2-yl)piperidine-2,6-dione (125 mg, 402.22 μmol, HCl salt) and tert-butyl (4-(2-oxoethyl)piperidin-1-yl)carbamate (146.19 mg, 603.32 μmol) in DCM (5 mL) were added TEA (40.70 mg, 402.22 μmol, 55.98 μL) and MgSO₄ (48.41 mg, 402.22 μmol). After stirring at 20° C. for 30 mins, NaBH(OAc)₃ (255.74 mg, 1.21 mmol) was added to the reaction mixture. The resulting mixture was stirred at 25° C. for 16 hr. LCMS showed 3-(5-(piperazin-1-yl)pyridin-2-yl)piperidine-2,6-dione was consumed completely and one main peak with desired mass. The residue was diluted with NaHCO₃ solution (10 mL) and extracted with DCM (10 mL×3). The combined organic layers were washed with water (10 mL), dried over Na₂SO₄, filtered and concentrated under reduced pressure. The residue was purified by flash silica gel chromatography (ISCO®; 10 g SepaFlash® Silica Flash Column, Eluent of 20-30% MeOH/EtOAc gradient @ 40 mL/min) to afford tert-butyl (4-(2-(4-(6-(2,6-dioxopiperidin-3-yl)pyridin-3-yl)piperazin-1-yl)ethyl)piperidin-1-yl)carbamate (140 mg, 279.65 μmol, 69.53% yield) as a yellow solid. MS(M+H)⁺=501.4.

### Step 6. Synthesis of 3-(5-(4-(2-(1-aminopiperidin-4-yl)ethyl)piperazin-1-yl)pyridin-2-yl)piperidine-2,6-dione (10)

To a solution of tert-butyl (4-(2-(4-(6-(2,6-dioxopiperidin-3-yl)pyridin-3-yl)piperazin-1-yl)ethyl)piperidin-1-yl)carbamate (140 mg, 279.65 μmol) in DCM (3 mL) was added TFA (1.54 g, 13.46 mmol, 1 mL). The mixture was stirred at 25° C. for 1 hr. LCMS showed the starting material was consumed completely and one main peak with desired mass. The reaction mixture was concentrated under reduced pressure to afford 3-(5-(4-(2-(1-aminopiperidin-4-yl)ethyl)piperazin-1-yl)pyridin-2-yl)piperidine-2,6-dione (144 mg, crude, TFA salt) as a brown oil. MS(M+H)⁺=401.2.

### Step 7. Synthesis of N-(2-chlorophenyl)-4-((2-((4-((4-(2-(4-(6-(2,6-dioxopiperidin-3-yl)pyridin-3-yl)piperazin-1-yl)ethyl)piperidin-1-yl)carbamoyl)phenyl)amino)-5-fluoropyrimidin-4-yl)amino)benzamide (Compound 88)

To a solution of 4-((4-((4-((2-chlorophenyl)carbamoyl)phenyl)amino)-5-fluoropyrimidin-2-yl)amino)benzoic acid (100 mg, 188.75 μmol) in DMF (2 mL) was added HATU (107.65 mg, 283.13 μmol) and DIPEA (121.97 mg, 943.76 μmol, 164.38 μL) and 3-(5-(4-(2-(1-aminopiperidin-4-yl)ethyl)piperazin-1-yl)pyridin-2-yl)piperidine-2,6-dione (126.26 mg, 245.38 μmol, TFA salt). The mixture was stirred at 25° C. for 2 hr. LCMS showed 3-(5-(4-(2-(1-aminopiperidin-4-yl)ethyl)piperazin-1-yl)pyridin-2-yl)piperidine-2,6-dione was consumed completely and one main peak with desired mass. The reaction mixture was mixed with water (10 mL) and filtered. The filter cake was collected to afford the crude product. The crude product was purified by prep-HPLC (column: Phenomenex luna C18 150*25 mm*10 um; mobile phase: [water(TFA)-ACN]; gradient: 15%-45% B over 10 min) and lyophilized to afford N-(2-chlorophenyl)-4-((2-((4-((4-(2-(4-(6-(2,6-dioxopiperidin-3-yl)pyridin-3-yl)piperazin-1-yl)ethyl)piperidin-1-yl)carbamoyl)phenyl)amino)-5-fluoropyrimidin-4-yl)amino)benzamide (42.7 mg, 32.74 μmol, 17.35% yield, 92.2% purity, 3TFA) as a white solid. MS(M+H)⁺=860.4.

¹H NMR (400 MHz, CD₃OD) δ=8.35 (d, J=2.9 Hz, 1H), 8.11 (d, J=4.0 Hz, 1H), 8.05-7.99 (m, 2H), 7.97-7.91 (m, 2H), 7.81-7.75 (m, 3H), 7.75-7.68 (m, 3H), 7.55-7.48 (m, 2H), 7.39 (dt, J=1.4, 7.7 Hz, 1H), 7.32-7.24 (m, 1H), 4.13-3.82 (m, 2H), 3.82-3.35 (m, 5H), 3.30-3.25 (m, 3H), 3.24-3.12 (m, 3H), 2.79-2.68 (m, 4H), 2.43-2.31 (m, 1H), 2.31-2.20 (m, 1H), 1.86-1.72 (m, 4H), 1.65-1.54 (m, 2H), 1.52-1.41 (m, 1H).

### Example 89. Synthesis of (S)-4-(4-(2-(1-(4-((4-((4-((2-chlorophenyl)carbamoyl)phenyl)amino)-5-fluoropyrimidin-2-yl)amino)benzamido)piperidin-4-yl)ethyl)piperazin-1-yl)-N-(2,6-dioxopiperidin-3-yl)-2-fluorobenzamide (Compound 89)

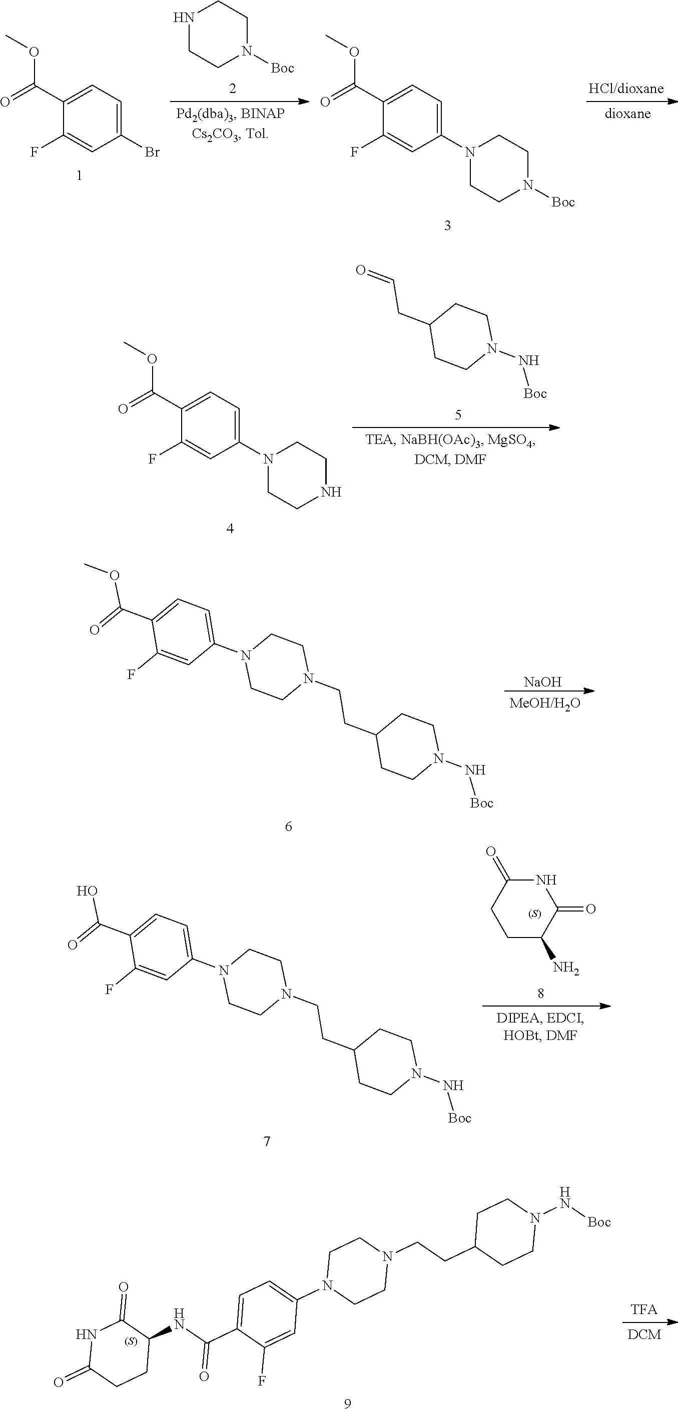

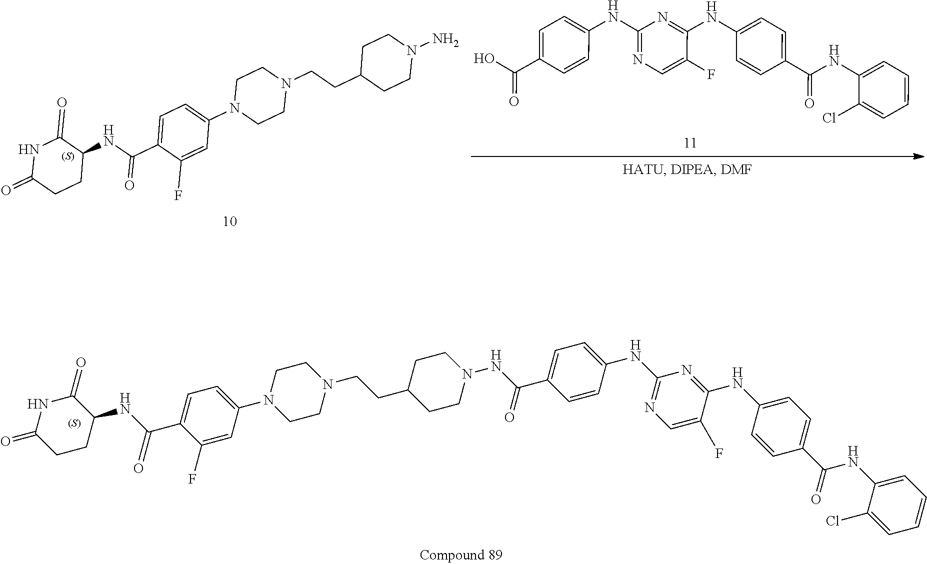

### Step 1. Synthesis of tert-butyl 4-(3-fluoro-4-(methoxycarbonyl)phenyl)piperazine-1-carboxylate (3)

A mixture of methyl 4-bromo-2-fluorobenzoate (5 g, 21.46 mmol), tert-butyl piperazine-1-carboxylate (4.00 g, 21.46 mmol), Cs₂CO₃ (20.97 g, 64.37 mmol), Pd₂ (dba)₃ (1.96 g, 2.15 mmol) and BINAP (1.34 g, 2.15 mmol) in toluene (50 mL) was degassed and purged with N₂ for 3 times, and then the mixture was stirred at 80° C. for 16 hr under N₂ atmosphere. LCMS showed methyl 4-bromo-2-fluorobenzoate was consumed completely and desired mass was detected. The mixture was filtered and the filter cake was washed with EtOAc (150 mL). The filtrate was concentrated under reduced pressure. The residue was purified by flash silica gel chromatography (ISCO®; 80 g SepaFlash® Silica Flash Column, Eluent of 10-13% EtOAc/Petroleum ether, gradient @ 100 mL/min) to afford tert-butyl 4-(3-fluoro-4-(methoxycarbonyl)phenyl)piperazine-1-carboxylate (1.56 g, 4.61 mmol, 21.49% yield) as a yellow solid. MS(M+H)⁺=339.2.

### Step 2. Synthesis of methyl 2-fluoro-4-(piperazin-1-yl)benzoate (4)

To a solution of tert-butyl 4-(3-fluoro-4-(methoxycarbonyl)phenyl)piperazine-1-carboxylate (1.56 g, 4.61 mmol) in dioxane (15 mL) was added HCl/dioxane (4 M, 1.15 mL). The mixture was stirred at 25° C. for 1 hr. LCMS showed the starting material was consumed completely and desired mass was detected. The reaction mixture was concentrated under reduced pressure to afford methyl 2-fluoro-4-(piperazin-1-yl)benzoate (1.27 g, crude, HCl) as a brown solid. MS(M+H)⁺=239.4.

### Step 3. Synthesis of methyl 4-(4-(2-(1-((tert-butoxycarbonyl)amino)piperidin-4-yl)ethyl)piperazin-1-yl)-2-fluorobenzoate (6)

To a solution of tert-butyl (4-(2-oxoethyl)piperidin-1-yl)carbamate (1.65 g, 6.81 mmol) in DCM (12 mL) were added a solution of methyl 2-fluoro-4-(piperazin-1-yl)benzoate (1.17 g, 4.26 mmol, HCl) in DMF (4 mL), TEA (2.15 g, 21.29 mmol, 2.96 mL) and MgSO₄ (1.54 g, 12.78 mmol). The mixture was stirred at 25° C. for 0.5 hr. And then NaBH(OAc)₃ (2.71 g, 12.78 mmol) was added in the reaction mixture and stirred at 25° C. for 16 hr. LC-MS showed the starting material was consumed completely and desired mass. The reaction mixture was diluted with H₂O (40 mL) and then extracted with EtOAc (40 mL×3). The combined organic layers were washed with brine (40 mL), dried over Na₂SO₄, filtered and concentrated under reduced pressure. The residue was purified by flash silica gel chromatography (ISCO®; 40 g SepaFlash® Silica Flash Column, Eluent of 75-100% EtOAc/Petroleum ether gradient @ 100 mL/min) to afford methyl 4-(4-(2-(1-((tert-butoxycarbonyl)amino)piperidin-4-yl)ethyl)piperazin-1-yl)-2-fluorobenzoate (1.23 g, 2.65 mmol, 62.17% yield) as a yellow solid. MS(M+H)⁺=465.4.

### Step 4. Synthesis of 4-(4-(2-(1-((tert-butoxycarbonyl)aminol)piperidin-4-yl)ethyl)piperazin-1-yl)-2-fluorobenzoic acid (7)

To a solution of methyl 4-(4-(2-(1-((tert-butoxycarbonyl)amino)piperidin-4-yl)ethyl)piperazin-1-yl)-2-fluorobenzoate (1 g, 2.15 mmol) in MeOH (10 mL) was added the solution of NaOH (258.28 mg, 6.46 mmol) in H₂O (2.5 mL). The mixture was stirred at 25° C. for 16 hr. LCMS showed the starting material was consumed completely and desired mass. The reaction mixture was concentrated under reduced pressure to remove MeOH. The residue was diluted with ACN (5 mL) and acidified to pH=5 with 2N HCl solution. The resulting mixture was filtered. The filter cake was collected and dried to afford 4-(4-(2-(1-((tert-butoxycarbonyl)amino)piperidin-4-yl)ethyl)piperazin-1-yl)-2-fluorobenzoic acid (1.4 g, crude) as a white solid. MS(M+H)⁺=451.3.

### Step 5. Synthesis of tert-butyl (S)-(4-(2-(4-(4-((2,6-dioxopiperidin-3-yl)carbamoyl)-3-fluorophenyl)piperazin-1-yl)ethyl)piperidin-1-yl)carbamate (9)

To a solution of 4-(4-(2-(1-((tert-butoxycarbonyl)amino)piperidin-4-yl)ethyl)piperazin-1-yl)-2-fluorobenzoic acid (0.5 g, 1.11 mmol) and (S)-3-aminopiperidine-2,6-dione (164.39 mg, 998.79 mol, HCl) in DMF (5 mL) were added HOBt (149.96 mg, 1.11 mmol), DIPEA (430.29 mg, 3.33 mmol, 579.90 μL) and EDCI (319.11 mg, 1.66 mmol). The mixture was stirred at 25° C. for 16 hr. LCMS showed 4-(4-(2-(1-((tert-butoxycarbonyl)amino)piperidin-4-yl)ethyl)piperazin-1-yl)-2-fluorobenzoic acid was consumed completely and desired mass was detected. The reaction mixture was diluted water (25 mL) at 25° C., and then extracted with EtOAc (80 mL×3). The combined organic layers were washed with brine (100 mL), dried over Na₂SO₄, filtered and concentrated under reduced pressure to give a residue. The residue was purified by flash silica gel chromatography (ISCO®; 20 g SepaFlash® Silica Flash Column, Eluent of 0-15% MeOH/EtOAc @ 80 mL/min) to afford tert-butyl (S)-(4-(2-(4-(4-((2,6-dioxopiperidin-3-yl)carbamoyl)-3-fluorophenyl)piperazin-1-yl)ethyl)piperidin-1-yl)carbamate (310 mg, 552.92 μmol, 49.82% yield) as a white solid. MS(M+H)⁺=561.4.

### Step 6. Synthesis of (S)-4-(4-(2-(1-aminopiperidin-4-yl)ethyl)piperazin-1-yl)-N-(2,6-dioxopiperidin-3-yl)-2-fluorobenzamide (10)

To a solution of tert-butyl (S)-(4-(2-(4-(4-((2,6-dioxopiperidin-3-yl)carbamoyl)-3-fluorophenyl)piperazin-1-yl)ethyl)piperidin-1-yl)carbamate (310 mg, 552.92 μmol) in DCM (3 mL) was added TFA (1.54 g, 13.46 mmol, 1 mL). The mixture was stirred at 25° C. for 1 hr. LCMS showed the starting material was consumed completely and desired mass. The reaction mixture was concentrated under reduced pressure to afford (S)-4-(4-(2-(1-aminopiperidin-4-yl)ethyl)piperazin-1-yl)-N-(2,6-dioxopiperidin-3-yl)-2-fluorobenzamide (317 mg, crude, TFA) as a yellow oil. MS(M+H)⁺=461.3.

### Step 7. Synthesis of (S)-4-(4-(2-(1-(4-((4-((4-((2-chlorophenyl)carbamoyl)phenyl)amino)-5-fluoropyrimidin-2-yl)amino)benzamido)piperidin-4-yl)ethyl)piperazin-1-yl)-N-(2,6-dioxopiperidin-3-yl)-2-fluorobenzamide (Compound 89)

To a solution of 4-((4-((4-((2-chlorophenyl)carbamoyl)phenyl)amino)-5-fluoropyrimidin-2-yl) amino)benzoic acid (145 mg, 303.43 μmol) in DMF (2 mL) were added HATU (173.06 mg, 455.14 μmol) and DIPEA (196.08 mg, 1.52 mmol, 264.26 μL). After stirring at 25° C. for 0.5 hr, (S)-4-(4-(2-(1-aminopiperidin-4-yl)ethyl)piperazin-1-yl)-N-(2,6-dioxopiperidin-3-yl)-2-fluorobenzamide (313.81 mg, 546.17 μmol, TFA) was added and the resulting mixture was stirred at 25° C. for 16 hr. LCMS showed (S)-4-(4-(2-(1-aminopiperidin-4-yl)ethyl)piperazin-1-yl)-N-(2,6-dioxopiperidin-3-yl)-2-fluorobenzamide was consumed completely and desired mass was detected. The reaction mixture was diluted with H₂O (20 mL) and extracted with EtOAc (20 mL×2). The combined organic layers were washed with brine (30 mL), dried over Na₂SO₄, filtered and concentrated under reduced pressure. The residue was triturated with DMF/DCM (2.5 mL, 1/5) at 25° C. for 1 hr. The crude product was purified by prep-HPLC (column: Phenomenex luna C18 150*25 mm*10 um; mobile phase: [water (TFA)-ACN]; gradient: 24%-54% B over 10 min) and lyophilized to afford (S)-4-(4-(2-(1-(4-((4-((4-((2-chlorophenyl)carbamoyl)phenyl)amino)-5-fluoropyrimidin-2-yl)amino)benzamido)piperidin-4-yl)ethyl)piperazin-1-yl)-N-(2,6-dioxopiperidin-3-yl)-2-fluorobenzamide (71.4 mg, 61.55 μmol, 20.28% yield, 99% purity, 2TFA) as a white solid. MS(M+H)⁺=920.1.

¹H NMR (400 MHz, DMSO-d₆) δ=10.85 (s, 1H), 9.93 (s, 1H), 9.82-9.73 (m, 1H), 9.72-9.54 (m, 2H), 8.25 (d, J=2.7 Hz, 1H), 8.18-8.10 (m, 1H), 8.08-7.96 (m, 4H), 7.85-7.76 (m, 2H), 7.75-7.61 (m, 4H), 7.57 (d, J=7.9 Hz, 1H), 7.40 (t, J=7.9 Hz, 1H), 7.35-7.26 (m, 1H), 7.01-6.85 (m, 2H), 4.80-4.67 (m, 1H), 4.10-4.03 (m, 2H), 3.58-3.54 (m, 2H), 3.24-3.07 (m, 8H), 2.89-2.72 (m, 3H), 2.60-2.55 (m, 1H), 2.22-2.07 (m, 1H), 2.06-1.95 (m, 1H), 1.82-1.75 (m, 2H), 1.70-1.59 (m, 2H), 1.47-1.30 (m, 3H).

### Example 90. Synthesis of 4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-(2-fluorophenyl)benzamide (Compound 90)

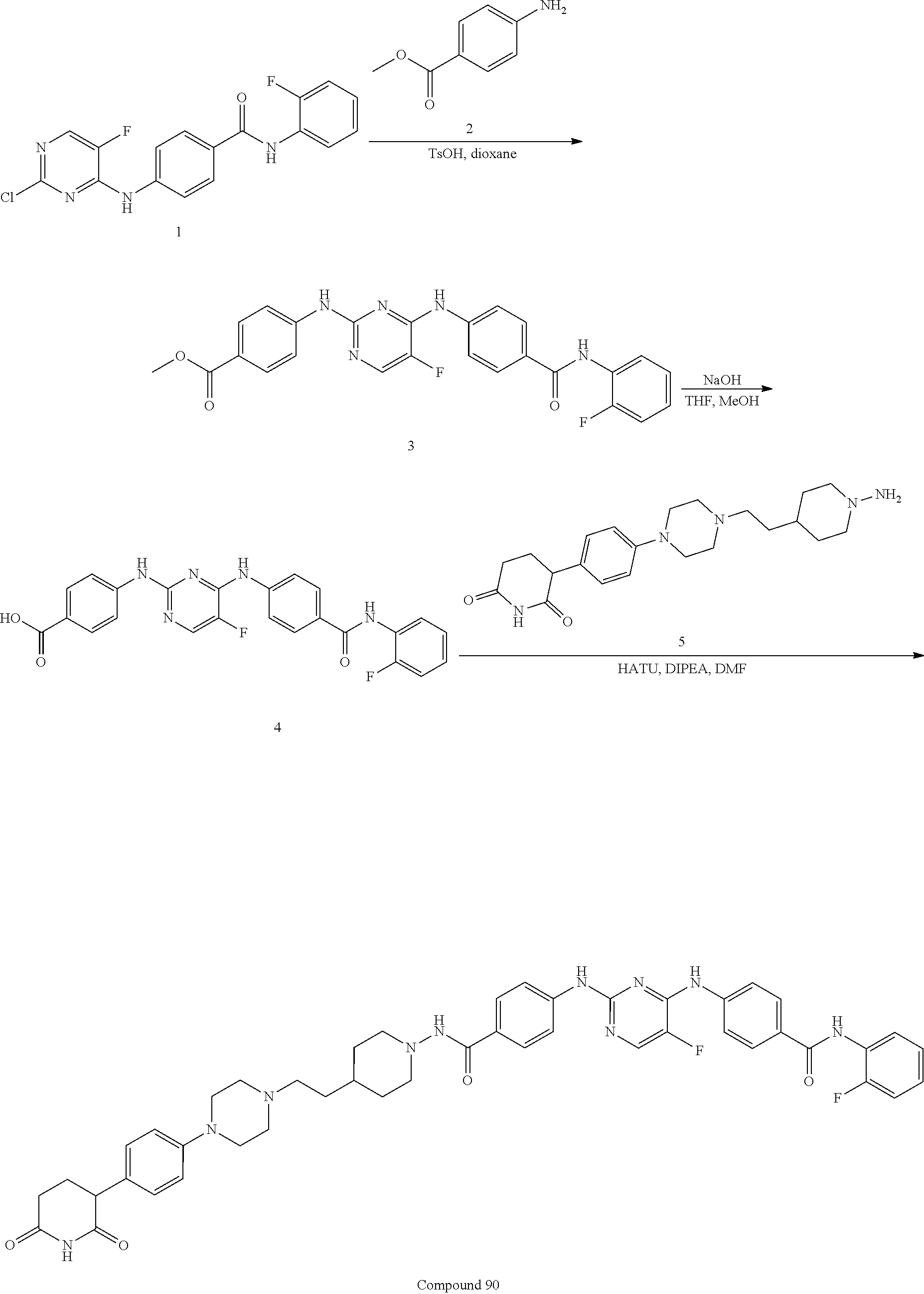

### Step 1. Synthesis of methyl 4-[[5-fluoro-4-[4-[(2-fluorophenyl)carbamoyl]anilino]pyrimidin-2-yl]amino]benzoate (3)

To a solution of 4-[(2-chloro-5-fluoro-pyrimidin-4-yl)amino]-N-(2-fluorophenyl)benzamide (500 mg, 1.39 mmol) and methyl 4-aminobenzoate (209.51 mg, 1.39 mmol) in dioxane (5 mL) was added TsOH·H₂O (790.93 mg, 4.16 mmol). The mixture was stirred at 100° C. for 16 hr. LCMS showed a peak (32%) with desired mass. The reaction mixture was diluted with 30 mL of water and filtered. The filter cake was collected and triturated with EtOAc/MeCN (10 mL, 1:1) to afford methyl 4-[[5-fluoro-4-[4-[(2-fluorophenyl)carbamoyl]anilino]pyrimidin-2-yl]amino]benzoate (350 mg, 301.82 μmol, 21.78% yield, 41% purity) as white solid. MS(M+H)⁺=476.1.

### Step 2. Synthesis of 4-[[5-fluoro-4-[4-[(2-fluorophenyl)carbamoyl]anilino]pyrimidin-2-yl]amino]benzoic acid (4)

To a solution of methyl 4-[[5-fluoro-4-[4-[(2-fluorophenyl)carbamoyl]anilino]pyrimidin-2-yl]amino]benzoate (350 mg, 736.15 μmol) in THF (12 mL) and MeOH (3 mL) was added NaOH (3 M, 3 mL). The resulting mixture was stirred at 40° C. for 24 hrs. LCMS showed the starting material was consumed and a peak (33%) with desired mass. The reaction mixture was neutralized to pH=7 with 1 M HCl solution and concentrated to remove organic solvent. The mixture was filtered and washed with 15 mL of MeOH. The filter cake was collected and dried to afford 4-[[5-fluoro-4-[4-[(2-fluorophenyl)carbamoyl]anilino]pyrimidin-2-yl]amino]benzoic acid (140 mg, 297.34 μmol, 40.39% yield, 98% purity) as white solid. MS(M+H)⁺=462.1.

### Step 3. Synthesis of 4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-(2-fluorophenyl)benzamide (Compound 90)

To a solution of 4-[[5-fluoro-4-[4-[(2-fluorophenyl)carbamoyl]anilino]pyrimidin-2-yl]amino]benzoic acid (90 mg, 195.05 μmol) and 3-[4-[4-[2-(1-amino-4-piperidyl)ethyl]piperazin-1-yl]phenyl]piperidine-2,6-dione (127.56 mg, 292.58 μmol, HCl) in DMF (2 mL) was added HATU (74.16 mg, 195.05 mol) and DIPEA (25.21 mg, 195.05 μmol, 33.97 μL). The mixture was stirred at 20° C. for 16 hr. LCMS showed a peak (38%) with desired mass. The reaction mixture was poured into water (10 mL) and filtered. The filter cake was collected to afford the crude product, which was purified by prep-HPLC (column: Phenomenex luna C18 150*25 mm*10 um; mobile phase: [water (TFA)-ACN]; gradient: 21%-51% B over 10 min) and lyophilized to afford 4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-(2-fluorophenyl)benzamide (37 mg, 30.60 μmol, 15.69% yield, 98% purity, 3TFA) as white solid. MS(M+H)⁺=843.4.

¹H NMR (400 MHz, DMSO-d₆) δ=10.79 (s, 1H), 10.01 (s, 1H), 9.76 (br s, 1H), 9.66 (br s, 1H), 9.63-9.43 (m, 1H), 8.25 (d, J=3.4 Hz, 1H), 8.07-7.94 (m, 4H), 7.84-7.68 (m, 4H), 7.67-7.56 (m, 1H), 7.36-7.18 (m, 3H), 7.12 (br d, J=8.6 Hz, 2H), 6.98 (br d, J=8.7 Hz, 2H), 3.85-3.83 (m, 3H), 3.80-3.75 (m, 2H), 3.24-3.09 (m, 6H), 3.00-2.91 (m, 2H), 2.87-2.76 (m, 2H), 2.72-2.58 (m, 2H), 2.22-2.07 (m, 1H), 2.05-1.93 (m, 1H), 1.81-1.59 (m, 4H), 1.47-1.27 (m, 3H).

### Example 91. Synthesis 6-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]pyridine-3-carboxamide (Compound 91)

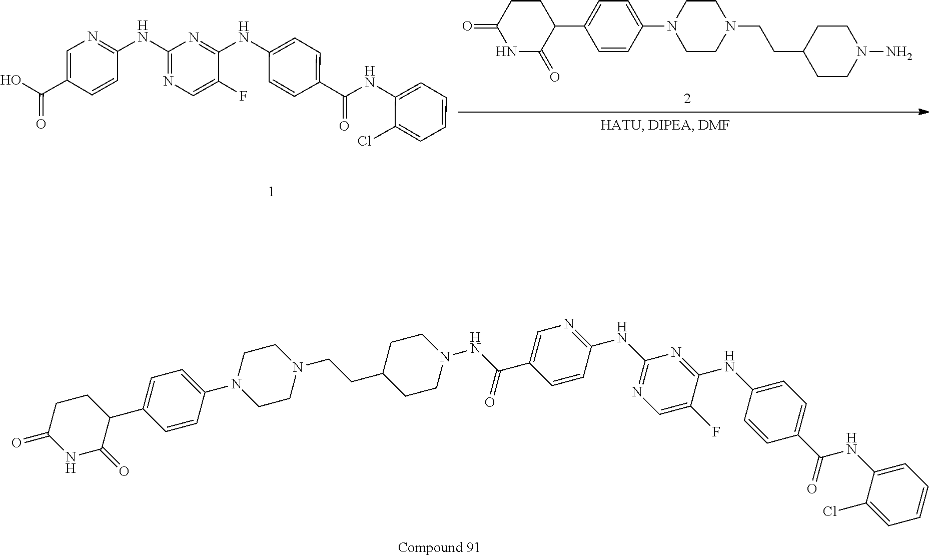 [SMILES: O=C(O)c1ccc(Nc2ncc(F)c(Nc3ccc(C(=O)Nc4ccccc4Cl)cc3)n2)nc1 ; NN1CCC(CCN2CCN(c3ccc(C4CCC(=O)NC4=O)cc3)CC2)CC1 ; O=C1CCC(c2ccc(N3CCN(CCC4CCN(NC(=O)c5ccc(Nc6ncc(F)c(Nc7ccc(C(=O)Nc8ccccc8Cl)cc7)n6)nc5)CC4)CC3)cc2)C(=O)N1]

### Step 1. Synthesis 6-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]pyridine-3-carboxamide (Compound 91)

To a solution of 6-[[4-[4-[(2-chlorophenyl)carbamoyl]anilino]-5-fluoro-pyrimidin-2-yl]amino]pyridine-3-carboxylic acid (150 mg, 313.24 μmol) and 3-[4-[4-[2-(1-amino-4-piperidyl)ethyl]piperazin-1-yl]phenyl]piperidine-2,6-dione (150.18 mg, 344.57 μmol, HCl) in DMF (3 mL) was added HATU (142.92 mg, 375.89 mol) and DIPEA (121.45 mg, 939.73 μmol, 163.68 μL). The mixture was stirred at 20° C. for 16 hr. LCMS showed a peak (31%) with desired mass. The reaction mixture was diluted with 20 mL of water and filtered to afford a residue. The residue was purified by prep-HPLC (column: Phenomenex luna C18 150*25 mm*10 um; mobile phase: [water (TFA)-ACN]; gradient: 20%-50% B over 10 min) and lyophilized to afford the impure product, which was further purified by prep-HPLC (column: Phenomenex luna C18 150*25 mm*10 um; mobile phase: [water (TFA)-ACN]; gradient: 19%-49% B over 10 min) and lyophilized to afford 6-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]pyridine-3-carboxamide (9 mg, 7.94 μmol, 2.53% yield, 96% purity, 2TFA) as white solid. MS(M+H)⁺=860.4.

¹H NMR (400 MHz, DMSO-d₆) δ=10.97-10.64 (m, 2H), 10.22-9.89 (m, 3H), 8.73 (s, 1H), 8.34 (d, J=3.7 Hz, 1H), 8.20 (br d, J=8.2 Hz, 1H), 8.15-8.07 (m, 2H), 8.06-7.95 (m, 3H), 7.68-7.52 (m, 2H), 7.40 (dt, J=1.3, 7.6 Hz, 1H), 7.30 (dt, J=1.6, 7.6 Hz, 1H), 7.12 (br d, J=8.6 Hz, 2H), 6.97 (br d, J=8.7 Hz, 2H), 3.85-3.75 (m, 3H), 3.22-3.01 (m, 10H), 2.89-2.77 (m, 2H), 2.72-2.57 (m, 2H), 2.22-2.08 (m, 1H), 2.07-1.92 (m, 1H), 1.84-1.60 (m, 4H), 1.43-1.36 (m, 3H).

### Example 92. Synthesis of N-cyclohexyl-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)benzamide (Compound 92)

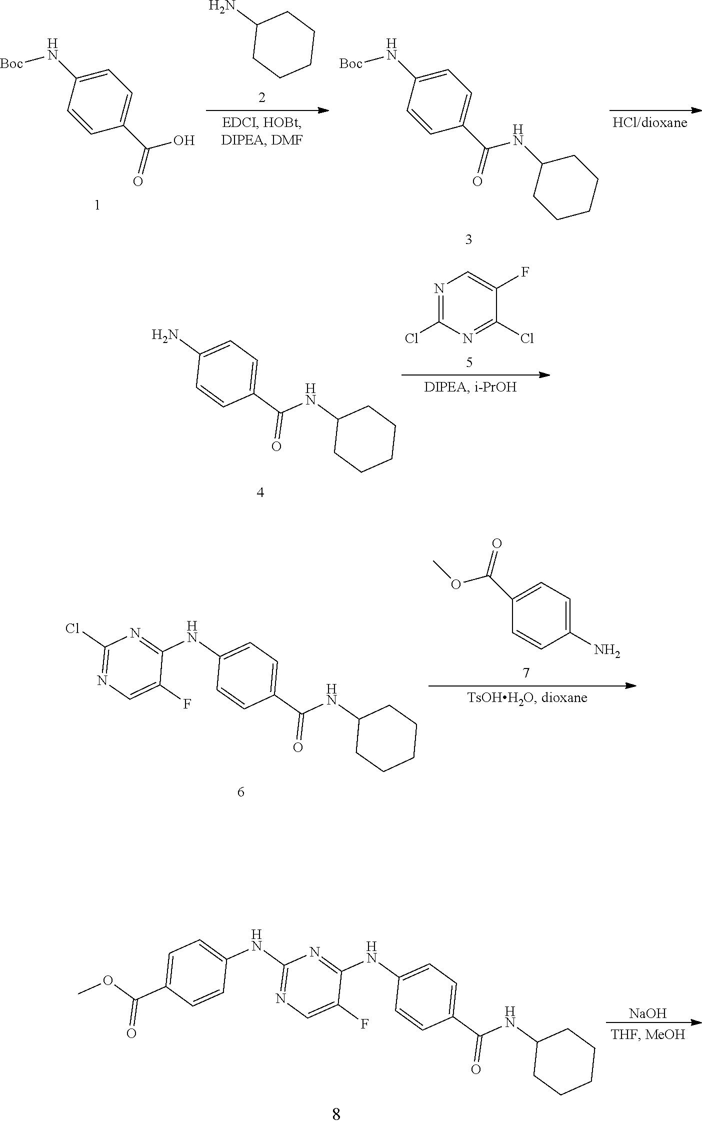

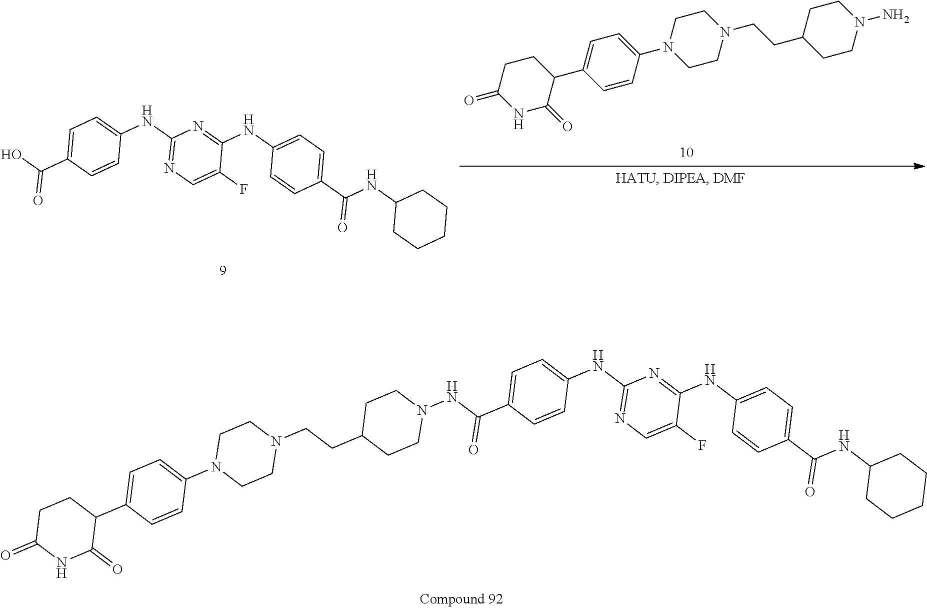 [SMILES: O=C(O)c1ccc(Nc2ncc(F)c(Nc3ccc(C(=O)NC4CCCCC4)cc3)n2)cc1 ; NN1CCC(CCN2CCN(c3ccc(C4CCC(=O)NC4=O)cc3)CC2)CC1 ; O=C1CCC(c2ccc(N3CCN(CCC4CCN(NC(=O)c5ccc(Nc6ncc(F)c(Nc7ccc(C(=O)NC8CCCCC8)cc7)n6)cc5)CC4)CC3)cc2)C(=O)N1]

### Step 1. Synthesis of tert-butyl (4-(cyclohexylcarbamoyl)phenyl)carbamate (3)

To a solution of 4-((tert-butoxycarbonyl)amino)benzoic acid (2 g, 8.43 mmol) and cyclohexanamine (1.25 g, 12.64 mmol, 1.45 mL) in DMF (20 mL) were added EDCI (2.42 g, 12.64 mmol), DIPEA (3.27 g, 25.29 mmol, 4.41 mL) and HOBt (2.28 g, 16.86 mmol). The mixture was stirred at 25° C. for 3 hr. LCMS showed 4-((tert-butoxycarbonyl)amino)benzoic acid was consumed completely and desired mass was detected. The reaction mixture was diluted with H₂O (30 mL) and EtOAc (30 mL). The mixture was filtered. The filter cake was collected and dried to afford tert-butyl (4-(cyclohexylcarbamoyl)phenyl)carbamate (2.5 g, crude) as a white solid. MS(M+H)⁺=319.2.

### Step 2. Synthesis of 4-amino-N-cyclohexylbenzamide (4)

A solution of tert-butyl (4-(cyclohexylcarbamoyl)phenyl)carbamate (1.5 g, 4.71 mmol) HCl/dioxane (2 M, 30 mL) was stirred at 25° C. for 3 hr. LCMS showed the starting material was consumed completely and desired mass was detected. The reaction mixture was concentrated under reduced pressure to afford 4-amino-N-cyclohexylbenzamide (1.2 g, crude, HCl) as a white solid. MS(M+H)⁺=219.1.

### Step 3. Synthesis of 4-((2-chloro-5-fluoropyrimidin-4-yl)amino)-N-cyclohexylbenzamide (6)

To a solution of 4-amino-N-cyclohexylbenzamide (1.2 g, 4.71 mmol, HCl) and 2,4-dichloro-5-fluoropyrimidine (2.20 g, 13.19 mmol) in i-PrOH (20 mL) was added DIPEA (1.83 g, 14.13 mmol, 2.46 mL). The mixture was stirred at 70° C. for 16 hr. LCMS showed a main peak with desired mass. The reaction mixture was diluted with H₂O (50 mL) and extracted with EtOAc (100 mL×2). The combined organic layers were dried over Na₂SO₄, filtered and concentrated under reduced pressure. The residue was purified by flash silica gel chromatography (ISCO®; 40 g SepaFlash® Silica Flash Column, Eluent of 0~50% EtOAc/Petroleum ether, gradient @ 100 mL/min) to afford 4-((2-chloro-5-fluoropyrimidin-4-yl)amino)-N-cyclohexylbenzamide (1.39 g, 3.99 mmol, 84.60% yield) as a yellow solid. MS(M+H)⁺=349.3.

### Step 4. Synthesis of methyl 4-((4-((4-(cyclohexylcarbamoyl)phenyl)amino)-5-fluoropyrimidin-2-yl)amino)benzoate (8)

To a solution of 4-((2-chloro-5-fluoropyrimidin-4-yl)amino)-N-cyclohexylbenzamide (390 mg, 1.12 mmol) and methyl 4-aminobenzoate (202.82 mg, 1.34 mmol) in dioxane (4 mL) was added TsOH·H₂O (638.05 mg, 3.35 mmol). The mixture was stirred at 100° C. for 16 hr. LCMS showed 4-((2-chloro-5-fluoropyrimidin-4-yl)amino)-N-cyclohexylbenzamide was consumed completely and desired mass. The reaction mixture was diluted with H₂O (20 mL) and extracted with EtOAc (50 mL×2). The combined organic layers were dried over Na₂ SO₄, filtered and concentrated under reduced pressure. The residue was purified by flash silica gel chromatography (ISCO®; 20 g SepaFlash® Silica Flash Column, Eluent of 70-100% EtOAc/Petroleum ether, gradient @ 100 mL/min) to afford methyl 4-((4-((4-(cyclohexylcarbamoyl)phenyl)amino)-5-fluoropyrimidin-2-yl)amino)benzoate (340 mg, 733.54 μmol, 65.61% yield) as a white solid. MS(M+H)⁺=464.3.

### Step 5. Synthesis of 4-((4-((4-(cyclohexylcarbamoyl)phenyl)amino)-5-fluoropyrimidin-2-yl)amino) benzoic acid (9)

To a solution of methyl 4-((4-((4-(cyclohexylcarbamoyl)phenyl)amino)-5-fluoropyrimidin-2-yl)amino)benzoate (340 mg, 733.54 μmol) in THF (4 mL) and MeOH (1 mL) was added NaOH (3 M, 1.22 mL). The mixture was stirred at 40° C. for 16 hr. LCMS showed the starting material was consumed completely and desired mass. The reaction mixture was concentrated under reduced pressure to remove THF and MeOH. The residue was acidified to pH=5 by 2N HCl solution and filtered. The filter cake was collected and dried to afford 4-((4-((4-(cyclohexylcarbamoyl)phenyl)amino)-5-fluoropyrimidin-2-yl)amino)benzoic acid (770 mg, crude) as a white solid. MS(M+H)⁺=450.2.

### Step 6. Synthesis of N-cyclohexyl-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)benzamide (Compound 92)

To a solution of 4-((4-((4-(cyclohexylcarbamoyl)phenyl)amino)-5-fluoropyrimidin-2-yl)amino) benzoic acid (185.57 mg, 412.85 μmol) in DMF (2 mL) were added HATU (130.82 mg, 344.05 μmol) and DIPEA (148.22 mg, 1.15 mmol, 199.75 μL). The mixture was stirred at 25° C. for 0.5 hr. Then 3-(4-(4-(2-(1-aminopiperidin-4-yl)ethyl)piperazin-1-yl)phenyl)piperidine-2,6-dione (100 mg, 229.36 μmol, HCl) was added and the resulting mixture was stirred at 25° C. for 0.5 hr. LCMS showed 3-(4-(4-(2-(1-aminopiperidin-4-yl)ethyl)piperazin-1-yl)phenyl)piperidine-2,6-dione was consumed completely and desired mass was detected. The reaction mixture was diluted with H₂O (15 mL) and filtered. The filter cake was collected and purified by prep-HPLC (column: Phenomenex luna C18 150*25 mm*10 um; mobile phase: [water (TFA)-ACN]; gradient: 20%-50% B over 10 min). The eluent was lyophilized to afford N-cyclohexyl-4-((2-((4-((4-(2-(4-(4-(2,6-dioxopiperidin-3-yl)phenyl)piperazin-1-yl)eth yl)piperidin-1-yl)carbamoyl)phenyl)amino)-5-fluoropyrimidin-4-yl)amino)benzamide (22.9 mg, 21.41 μmol, 9.33% yield, 99% purity, 2TFA) as a white solid. MS(M+H)⁺=831.2.

¹H NMR (400 MHz, DMSO-d₆) δ=10.79 (s, 1H), 9.65-9.62 (m, 2H), 9.56-9.35 (m, 2H), 8.22 (d, J=3.5 Hz, 1H), 8.09 (d, J=8.2 Hz, 1H), 7.93-7.84 (m, 4H), 7.80-7.74 (m, 2H), 7.71-7.67 (m, 2H), 7.12 (d, J=8.7 Hz, 2H), 6.98 (br d, J=8.8 Hz, 2H), 3.86-3.83 (m, 1H), 3.82-3.78 (m, 2H), 3.24-3.18 (m, 2H), 3.17-3.08 (m, 4H), 3.00-2.92 (m, 2H), 2.85-2.76 (m, 2H), 2.68-2.60 (m, 1H), 2.47-2.44 (m, 2H), 2.20-2.11 (m, 1H), 2.05-1.96 (m, 1H), 1.87-1.59 (m, 10H), 1.44-1.23 (m, 8H), 1.19-1.09 (m, 1H).

### Example 93. Synthesis of 2-chloro-N-(4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)phenyl)benzamide (Compound 93)

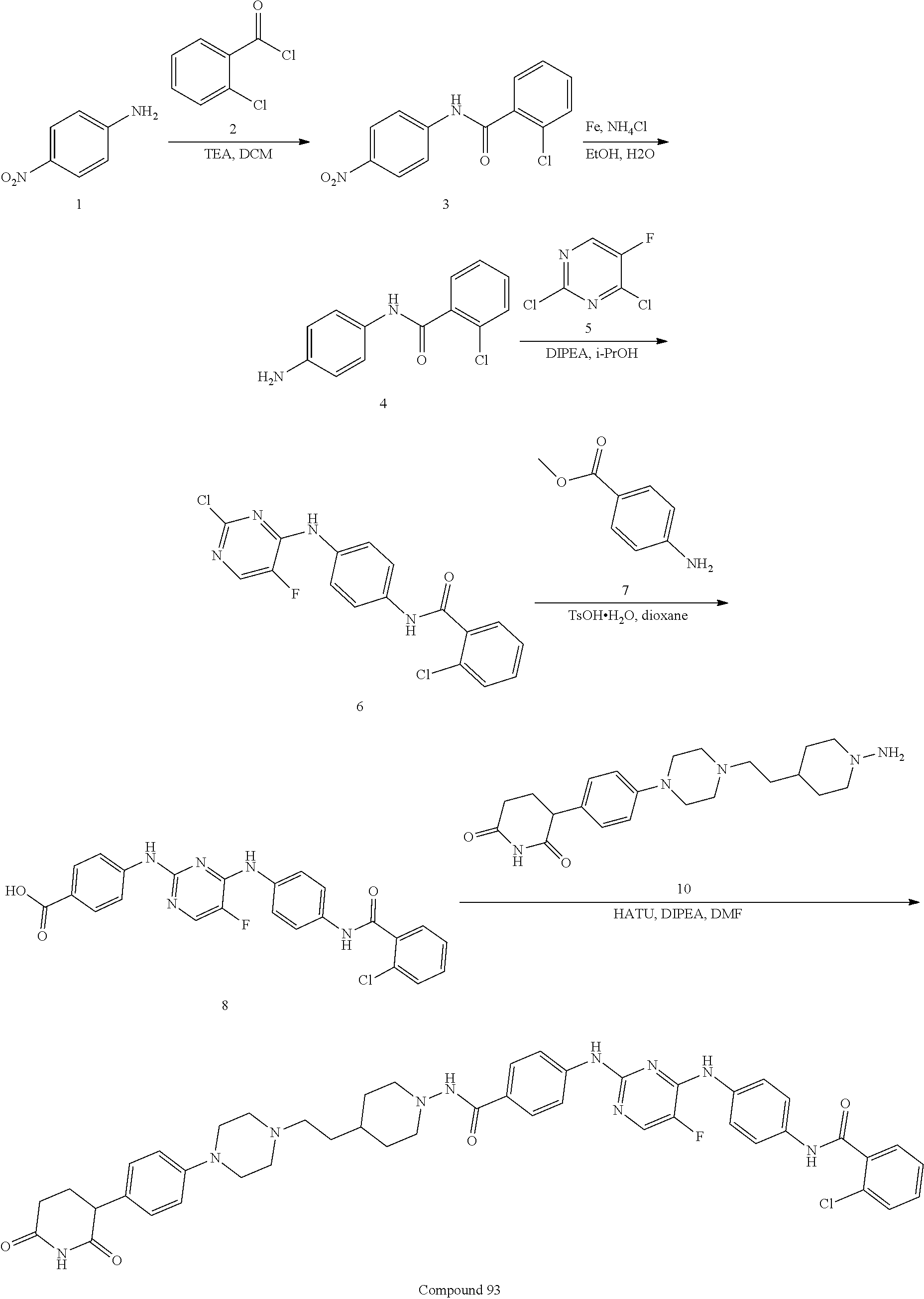

### Step 1. Synthesis of 2-chloro-N-(4-nitrophenyl)benzamide (3)

To a solution of 4-nitroaniline (1.58 g, 11.43 mmol, 1.10 mL) and TEA (1.73 g, 17.14 mmol, 2.39 mL) in DCM (20 mL) was added the solution of 2-chlorobenzoyl chloride (2 g, 11.43 mmol, 1.68 mL) in DCM (10 mL) slowly at 0° C. The mixture was stirred at 25° C. for 16 hr. LCMS showed 25% of 4-nitroaniline remained. Then a solution of 2-chlorobenzoyl chloride (400.00 mg, 2.29 mmol, 336.13 μL) in DCM (2 mL) was added in the reaction mixture and stirred at 25° C. for 3 hr. LCMS showed 23% of 4-nitroaniline remained. Then TEA (1.73 g, 17.14 mmol, 2.39 mL) was added ad the reaction mixture and stirred at 25° C. for 16 hr. LC-MS showed 24% of 4-nitroaniline remained. The reaction mixture was diluted with H₂O (80 mL) and extracted with EtOAc (80 mL×3). The combined organic layers were dried over Na₂ SO₄, filtered and concentrated under reduced pressure. The residue was purified by flash silica gel chromatography (ISCO®; 80 g SepaFlash® Silica Flash Column, Eluent of 0-70% EtOAc/Petroleum ether, gradient @ 100 mL/min) to afford 2-chloro-N-(4-nitrophenyl)benzamide (670 mg, 2.42 mmol, 21.19% yield) as a yellow solid. MS(M+H)⁺=277.1.

### Step 2. Synthesis of N-(4-aminophenyl)-2-chlorobenzamide (4)

To a solution of 2-chloro-N-(4-nitrophenyl)benzamide (670 mg, 2.42 mmol) in EtOH (15 mL) and H₂O (3 mL) were added NH₄Cl (1.30 g, 24.22 mmol) and Fe (676.18 mg, 12.11 mmol). The resulting mixture was stirred at 80° C. for 1 hr. LCMS showed the starting material was consumed completely and desired mass was detected. The reaction mixture was diluted with saturated Na₂CO₃ solution (30 mL) and extracted with EtOAc (40 mL×2). The combined organic layers were dried over Na₂SO₄, filtered and concentrated under reduced pressure to afford N-(4-aminophenyl)-2-chlorobenzamide (590 mg, crude) as a brown oil. MS(M+H)⁺=247.1.

### Step 3. Synthesis of 2-chloro-N-(4-((2-chloro-5-fluoropyrimidin-4-yl)amino)phenyl)benzamide (6)

To a solution of N-(4-aminophenyl)-2-chlorobenzamide (590 mg, 2.39 mmol) and 2,4-dichloro-5-fluoropyrimidine (1.12 g, 6.70 mmol) in i-PrOH (6 mL) was added DIPEA (927.31 mg, 7.17 mmol, 1.25 mL). The mixture was stirred at 70° C. for 16 hr. LCMS showed N-(4-aminophenyl)-2-chlorobenzamide was consumed completely and desired mass was detected. The reaction mixture was diluted with H₂O (50 mL) and extracted with EtOAc (60 mL×2). The combined organic layers were dried over Na₂ SO₄, filtered and concentrated under reduced pressure. The residue was purified by flash silica gel chromatography (ISCO®; 20 g SepaFlash® Silica Flash Column, Eluent of 20~50% EtOAc/Petroleum ether, gradient @ 100 mL/min) to afford 2-chloro-N-(4-((2-chloro-5-fluoropyrimidin-4-yl)amino)phenyl)benzamide (810 mg, 2.15 mmol, 89.79% yield) as a yellow solid. MS(M+H)⁺=377.1.

### Step 4. Synthesis of 4-((4-((4-(2-chlorobenzamido)phenyl)amino)-5-fluoropyrimidin-2-yl)amino) benzoic acid (8)

To a solution of 2-chloro-N-(4-((2-chloro-5-fluoropyrimidin-4-yl)amino)phenyl)benzamide (400 mg, 1.06 mmol) and methyl 4-aminobenzoate (192.36 mg, 1.27 mmol) in dioxane (4 mL) was added TsOH·H₂O (605.15 mg, 3.18 mmol). The mixture was stirred at 100° C. for 16 hr. LCMS showed 2-chloro-N-(4-((2-chloro-5-fluoropyrimidin-4-yl)amino)phenyl)benzamide was consumed completely and desired mass was detected. The reaction mixture was diluted with H₂O (20 mL) and EtOAc (20 mL). The mixture was filtered. The filter cake was collected and dried to afford 4-((4-((4-(2-chlorobenzamido)phenyl)amino)-5-fluoropyrimidin-2-yl)amino)benzoic acid (160 mg, crude) as a white solid. MS(M+H)⁺=478.2.

### Step 5. Synthesis of 2-chloro-N-(4-((2-((4-((4-(2-(4-(4-(2,6-dioxopiperidin-3-yl)phenyl)piperazin-1-yl)ethyl)piperidin-1-yl)carbamoylphenyl)amino)-5-fluoropyrimidin-4-yl)amino)phenyl) benzamide (Compound 93)

To a solution of 4-((4-((4-(2-chlorobenzamido)phenyl)amino)-5-fluoropyrimidin-2-yl)amino) benzoic acid (80 mg, 167.41 μmol) in DMF (2 mL) were added HATU (95.48 mg, 251.11 μmol) and DIPEA (108.18 mg, 837.04 μmol, 145.80 μL). After stirring at 25° C. for 0.5 hr, 3-(4-(4-(2-(1-aminopiperidin-4-yl)ethyl)piperazin-1-yl)phenyl)piperidine-2,6-dione (131.38 mg, 301.33 μmol, HCl) was added and the resulting mixture was stirred at 25° C. for 16 hr. LCMS showed 4-((4-((4-(2-chlorobenzamido)phenyl)amino)-5-fluoropyrimidin-2-yl)amino)benzoic acid was consumed completely and desired mass was detected. The reaction mixture was diluted with H₂O (10 mL) and filtered. The filter cake was collected and purified by prep-HPLC (column: Phenomenex luna C18 150*25 mm*10 um; mobile phase: [water (TFA)-ACN]; gradient: 17%-47% B over 10 min). The eluent was lyophilized to afford 2-chloro-N-(4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl) amino)phenyl)benzamide (61 mg, 54.97 μmol, 32.84% yield, 98% purity, 2TFA) as a white solid. MS(M+H)⁺=859.1.

¹H NMR (400 MHz, DMSO-d₆) δ=10.79 (s, 1H), 10.52 (s, 1H), 9.58-9.48 (m, 4H), 8.15 (d, J=3.7 Hz, 1H), 7.79-7.65 (m, 8H), 7.63-7.56 (m, 2H), 7.55-7.44 (m, 2H), 7.12 (d, J=8.6 Hz, 2H), 6.98 (br d, J=8.7 Hz, 2H), 3.78-3.75 (m, 1H), 3.75-3.72 (m, 2H), 3.63-3.58 (m, 2H), 3.25-3.07 (m, 6H), 3.01-2.91 (m, 2H), 2.85-2.74 (m, 2H), 2.70-2.59 (m, 1H), 2.48-2.42 (m, 1H), 2.20-2.11 (m, 1H), 2.05-1.95 (m, 1H), 1.78-1.70 (m, 2H), 1.68-1.60 (m, 2H), 1.42-1.31 (m, 3H).

### Example 94. Synthesis of (S)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoro pyrimidin-4-yl)amino)benzamide (Compound 94)

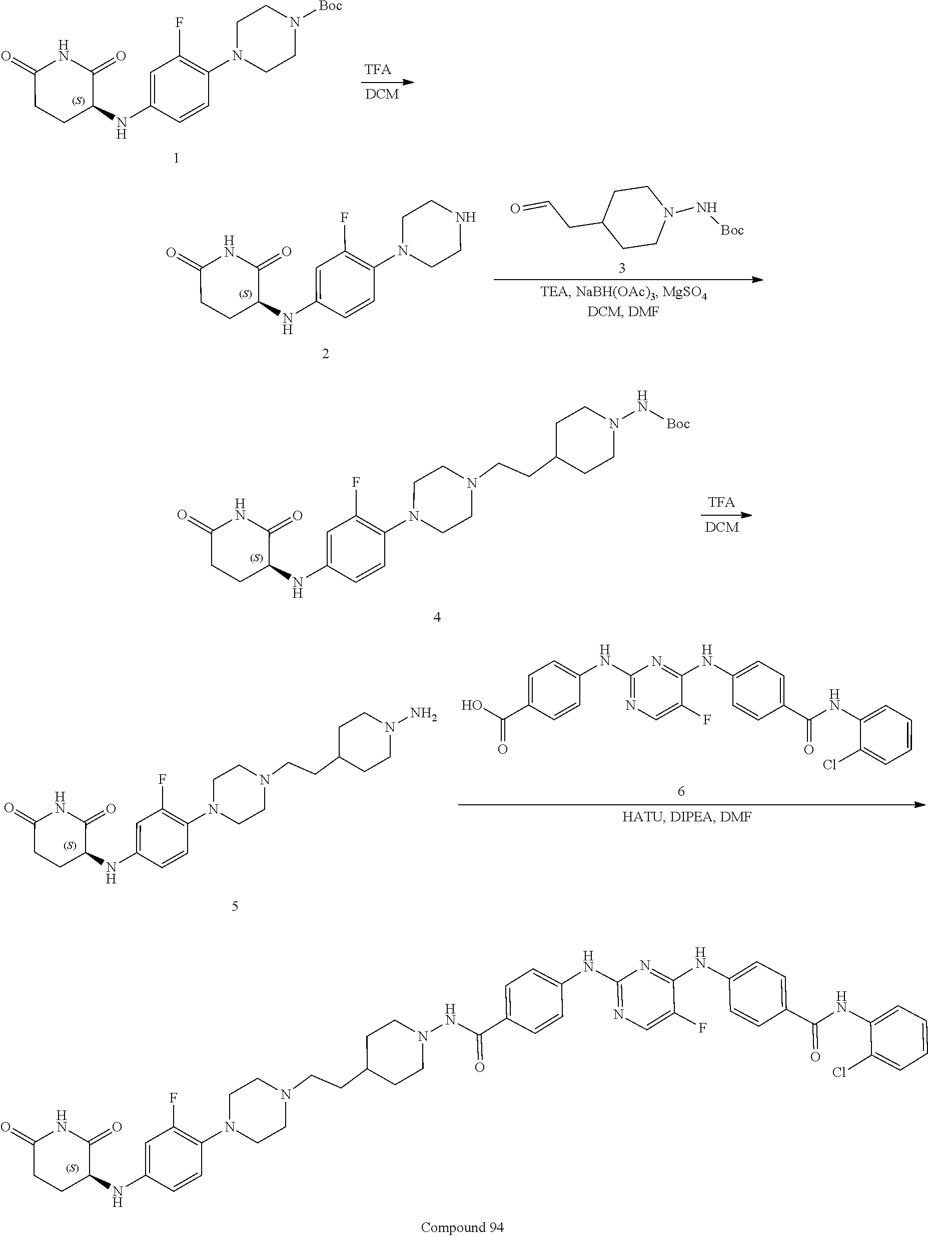

### Step 1. Synthesis of (S)-3-((3-fluoro-4-(piperazin-1-yl)phenyl)amino)piperidine-2,6-dione (2)

To a solution of tert-butyl (S)-4-(4-((2,6-dioxopiperidin-3-yl)amino)-2-fluorophenyl)piperazine-1-carboxylate (200 mg, 492.07 μmol) in DCM (2 mL) was added TFA (921.00 mg, 8.08 mmol, 0.6 mL) at 0° C. and the mixture was stirred at 0° C. for 1 h. LCMS showed a main peak with desired mass. The mixture was concentrated in vacuum to afford (S)-3-((3-fluoro-4-(piperazin-1-yl)phenyl)amino)piperidine-2,6-dione (200 mg, 475.79 μmol, 96.69% yield, TFA salt) as a brown oil. MS(M+H)⁺=307.1.

### Step 2. Synthesis of tert-butyl (S)-(4-(2-(4-(4-((2,6-dioxopiperidin-3-yl)amino)-2-fluorophenyl)piperazin-1-yl)ethyl)piperidin-1-yl)carbamate (4)

To a solution of (S)-3-((3-fluoro-4-(piperazin-1-yl)phenyl)amino)piperidine-2,6-dione (200 mg, 475.79 μmol, TFA salt) in DCM (3 mL) were added TEA (203.56 mg, 2.01 mmol, 280 μL) and MgSO₄ (137.45 mg, 1.14 mmol) and then followed by a solution of tert-butyl (4-(2-oxoethyl)piperidin-1-yl)carbamate (150 mg, 619.03 μmol) in DMF (3 mL) and the mixture was stirred at 0° C. for 30 min. NaBH(OAc)₃ (201.68 mg, 951.57 μmol) was added. The resulting mixture was stirred at 0° C. for 1 h. LCMS showed a peak (74%) with desired mass. The reaction mixture was diluted with NaHCO₃ (10 mL). The organic phase was separated, the aqueous was extracted with EtOAc (10 mL×3). The combined organic layers were washed with brine (30 mL×2), dried over Na₂SO₄, filtered, and the filtrate was concentrated under reduced pressure. The residue was purified by flash silica gel chromatography (Biotage; 10 g SepaFlash® Silica Flash Column, Eluent of 50-100% EtOAc/Petroleum ether to 10% MeOH/EtOAc gradient @ 80 mL/min) to afford tert-butyl (S)-(4-(2-(4-(4-((2,6-dioxopiperidin-3-yl)amino)-2-fluorophenyl)piperazin-1-yl)ethyl)piperidin-1-yl)carbamate (150 mg, 281.61 μmol, 59.19% yield) as a yellow oil. MS(M+H)⁺=533.3.

### Step 3. Synthesis of (S)-3-((4-(4-(2-(1-aminopiperidin-4-yl)ethyl)piperazin-1-yl)-3-fluorophenyl)amino)piperidine-2,6-dione (5)

To a solution of tert-butyl (S)-(4-(2-(4-(4-((2,6-dioxopiperidin-3-yl)amino)-2-fluorophenyl)piperazin-1-yl)ethyl)piperidin-1-yl)carbamate (150 mg, 281.61 μmol) in DCM (1.5 mL) was added TFA (767.50 mg, 6.73 mmol, 0.5 mL), the mixture was stirred at 25° C. for 1 h. LCMS showed a main peak with desired mass. The mixture was concentrated in vacuum to afford (S)-3-((4-(4-(2-(1-aminopiperidin-4-yl)ethyl)piperazin-1-yl)-3-fluorophenyl)amino)piperidine-2,6-dione (150 mg, crude, TFA salt) as a yellow oil. MS(M+H)⁺=433.2.

### Step 4. Synthesis of (S)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (Compound 94)

To a solution of 4-((4-((4-((2-chlorophenyl)carbamoyl)phenyl)amino)-5-fluoropyrimidin-2-yl)amino)benzoic acid (131.15 mg, 274.45 μmol) in DMF (2 mL) were added HATU (125.22 mg, 329.33 μmol), DIPEA (106.41 mg, 823.34 μmol, 143.41 μL), the mixture was stirred at 25° C. for 0.5 h. Then (S)-3-((4-(4-(2-(1-aminopiperidin-4-yl)ethyl)piperazin-1-yl)-3-fluorophenyl) amino)piperidine-2,6-dione (150 mg, 274.45 μmol, TFA salt) was added and the resulting mixture was stirred at 25° C. for 1 h. LCMS showed a peak (79%) with desired mass. The reaction mixture was poured into ice water (10 mL) and filtered. The filter cake was collected and dried to afford the crude product. The crude product was purified by prep-HPLC (column: Phenomenex luna C18 150*25 mm*10 um; mobile phase: [water (TFA)-ACN]; gradient: 23%-53% B over 10 min) and lyophilized to afford (S)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (125.7 mg, 111.07 μmol, 40.47% yield, 99% purity, 2 TFA) as a white solid. MS(M+H)⁺=892.1.

¹H NMR (400 MHz, DMSO-d₆) δ=10.79 (s, 1H), 9.94 (s, 1H), 9.83-9.63 (m, 3H), 9.56-9.44 (m, 1H), 8.26 (d, J=3.4 Hz, 1H), 8.06-7.98 (m, 4H), 7.84-7.77 (m, 2H), 7.75-7.69 (m, 2H), 7.68-7.62 (m, 1H), 7.60-7.54 (m, 1H), 7.44-7.37 (m, 1H), 7.34-7.27 (m, 1H), 6.94-6.86 (m, 1H), 6.60-6.51 (m, 1H), 6.46 (d, J=8.9 Hz, 1H), 4.29 (d, J=6.5 Hz, 1H), 3.56 (d, J=10.8 Hz, 2H), 3.30-3.13 (m, 8H), 3.00-2.91 (m, 2H), 2.88-2.76 (m, 2H), 2.78-2.68 (m, 1H), 2.62-2.58 (m, 1H), 2.12-2.03 (m, 1H), 1.94-1.82 (m, 1H), 1.80-1.65 (m, 2H), 1.65-1.55 (m, 2H), 1.46-1.36 (m, 3H).

### Example 95. Synthesis of (S)—N-(2-chlorophenyl)-4-((2-((4-((4-(2-(4-(4-((2,6-dioxopiperidin-3-yl)amino)-3-fluorophenyl)piperazin-1-yl)ethyl)piperidin-1-yl)carbamoyl)phenyl)amino)-5-fluoro pyrimidin-4-yl)amino)benzamide (Compound 95)

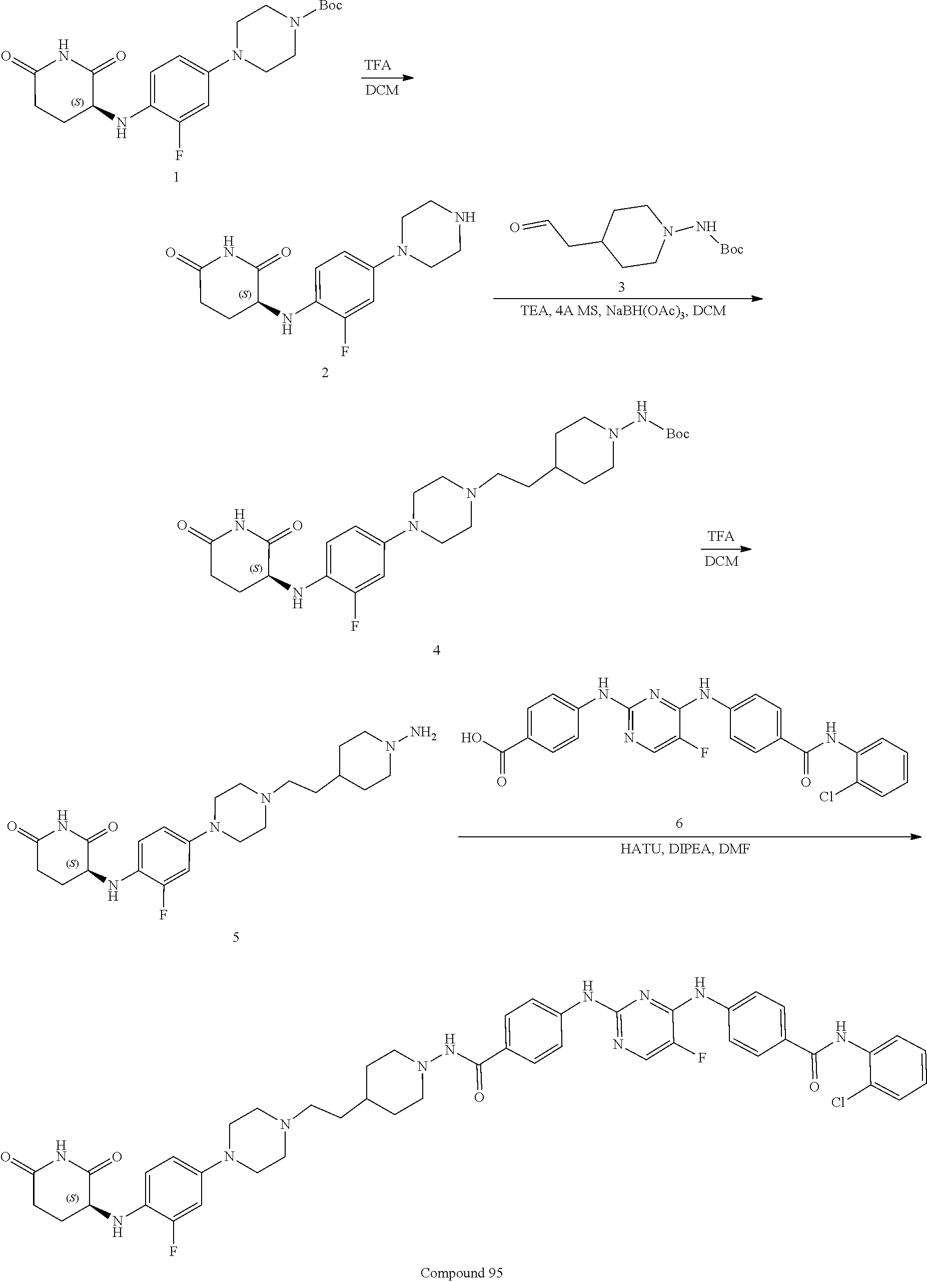

### Step 1. Synthesis of (S)-3-((2-fluoro-4-(piperazin-1-yl)phenyl)amino)piperidine-2,6-dione (2)

To a solution of tert-butyl (S)-4-(4-((2,6-dioxopiperidin-3-yl)amino)-3-fluorophenyl)piperazine-1-carboxylate (250.00 mg, 615.08 μmol) in DCM (2 mL) was added TFA (767.50 mg, 6.73 mmol, 0.5 mL) at 0° C. and the mixture was stirred at 20° C. for 16 h. LCMS showed a main peak with desired mass. The mixture was concentrated under reduced pressure to afford (S)-3-((2-fluoro-4-(piperazin-1-yl)phenyl)amino)piperidine-2,6-dione (260 mg, crude, TFA) as yellow oil. MS(M+H)⁺=307.2.

### Step 2. Synthesis of tert-butyl (S)-(4-(2-(4-(4-((2,6-dioxopiperidin-3-yl)amino)-3-fluorophenyl)piperazin-1-yl)ethyl)piperidin-1-yl)carbamate (4)

To a solution of (S)-3-((2-fluoro-4-(piperazin-1-yl)phenyl)amino)piperidine-2,6-dione (250.00 mg, 594.73 μmol, TFA salt) in DCM (4 mL) were added TEA (120.36 mg, 1.19 mmol, 165.56 μL), 4 Å MS (250 mg) and tert-butyl (4-(2-oxoethyl)piperidin-1-yl)carbamate (216.17 mg, 892.10 μmol). After stirring at 20° C. for 0.5 h, NaBH(OAc)₃ (252.10 mg, 1.19 mmol) was added. The resulting mixture was stirred at 20° C. for 13 h. LCMS showed a peak (51%) with desired mass. The residue was diluted with NaHCO₃ solution (20 mL) and extracted with DCM (20 mL×3). The combined organic layers were washed with water (30 mL), dried over Na₂SO₄, filtered and concentrated under reduced pressure. The residue was purified by flash silica gel chromatography (ISCO®; 10 g SepaFlash® Silica Flash Column, Eluent of 0-10% MeOH/EtOAc gradient @ 80 mL/min) to afford tert-butyl (S)-(4-(2-(4-(4-((2,6-dioxopiperidin-3-yl)amino)-3-fluorophenyl)piperazin-1-yl)ethyl)piperidin-1-yl)carbamate (260 mg, 395.38 μmol, 66.48% yield, 81% purity) as an yellow solid. MS(M+H)⁺=533.4.

### Step 3. Synthesis of (S)-3-((4-(4-(2-(1-aminopiperidin-4-yl)ethyl)piperazin-1-yl)-2-fluorophenyl) amino)piperidine-2,6-dione (5)

To a solution of tert-butyl (S)-(4-(2-(4-(4-((2,6-dioxopiperidin-3-yl)amino)-3-fluorophenyl)piperazin-1-yl)ethyl)piperidin-1-yl)carbamate (260.00 mg, 488.13 μmol) in DCM (5 mL) was added TFA (1.54 g, 13.46 mmol, 1 mL) and the mixture was stirred at 25° C. for 1 h. LCMS showed a main peak (94%) with desired mass. The mixture was concentrated under reduced pressure to afford (S)-3-((4-(4-(2-(1-aminopiperidin-4-yl)ethyl)piperazin-1-yl)-2-fluorophenyl) amino)piperidine-2,6-dione (260 mg, crude, TFA) as brown oil. MS(M+H)⁺=433.3.

### Step 4. Synthesis of (S)—N-(2-chlorophenyl)-4-((2-((4-((4-(2-(4-(4-((2,6-dioxopiperidin-3-yl)amino)-3-fluorophenyl)piperazin-1-ylethyl)piperidin-1-yl)carbamoyl)phenyl)amino)-5-fluoropyrimidin-4-yl)amino)benzamide (Compound 95)

To a solution of 4-((4-((4-((2-chlorophenyl)carbamoyl)phenyl)amino)-5-fluoropyrimidin-2-yl) amino)benzoic acid (227.33 mg, 475.71 μmol) in DMF (3 mL) were added HATU (217.05 mg, 570.85 μmol) and DIPEA (184.44 mg, 1.43 mmol, 248.58 μL) and the mixture was stirred at 20° C. for 0.5 h. Then (S)-3-((4-(4-(2-(1-aminopiperidin-4-yl)ethyl)piperazin-1-yl)-2-fluorophenyl)amino)piperidine-2,6-dione (260.00 mg, 475.71 μmol, TFA salt) was added and the resulting mixture was stirred at 20° C. for 12 h. LCMS showed a peak (57%) with desired mass. The reaction mixture was diluted with water (20 mL) and extracted with EtOAc (10 mL×3). The combined organic layers were washed with saturated brine (10 mL), dried over anhydrous Na₂SO₄, filtered and concentrated under reduced pressure. The residue was purified by prep-HPLC (column: Phenomenex luna C18 150*25 mm*10 um; mobile phase: [water (TFA)-ACN]; gradient: 21%-51% B over 10 min) and lyophilized to afford 100 mg of the crude product. The crude product was re-purified by prep-HPLC (column: Waters xbridge 150*25 mm 10 um; mobile phase: [water (NH₄HCO₃)-ACN]; gradient: 40%-60% B over 8 min) and lyophilized to afford 30 mg of crude product. The crude product was re-purified by prep-HPLC ((column: Phenomenex luna C18 150*25 mm*10 um; mobile phase: [water(TFA)-ACN]; gradient: 21%-51% B over 10 min) and lyophilized to afford (S)—N-(2-chlorophenyl)-4-((2-((4-((4-(2-(4-(4-((2,6-dioxopiperidin-3-yl)amino)-3-fluorophenyl)piperazin-1-yl)ethyl)piperidin-1-yl)carbamoyl)phenyl)amino)-5-fluoropyrimidin-4-yl)amino)benzamide (18.7 mg, 16.72 μmol, 49.74% yield, 90% purity, TFA) as a red solid. MS(M+H)⁺=892.3.

¹H NMR (400 MHz, DMSO-d₆) δ=10.81 (s, 1H), 9.94 (s, 1H), 9.78 (s, 1H), 9.67 (s, 1H), 9.62-9.55 (m, 1H), 9.44-9.33 (m, 1H), 8.26 (d, J=3.4 Hz, 1H), 8.06-8.00 (m, 4H), 7.83-7.76 (m, 2H), 7.75-7.70 (m, 2H), 7.66 (d, J=7.2 Hz, 1H), 7.58 (d, J=8.6 Hz, 1H), 7.41 (t, J=7.8 Hz, 1H), 7.34-7.28 (m, 1H), 6.88 (br d, J=15.9 Hz, 1H), 6.79 (t, J=9.4 Hz, 1H), 6.72-6.63 (m, 1H), 4.32 (br dd, J=4.8, 11.4 Hz, 1H), 3.62-3.56 (m, 4H), 3.26-3.09 (m, 6H), 2.91-2.64 (m, 5H), 2.62-2.56 (m, 1H), 2.15-1.94 (m, 2H), 1.79-1.71 (m, 2H), 1.69-1.60 (m, 2H), 1.44-1.32 (m, 3H).

### Example 96. Synthesis of N-(2-chlorophenyl)-4-((2-((4-((4-(2-(1-(4-(2,6-dioxopiperidin-3-yl)-2-fluorophenyl)-4-hydroxypiperidin-4-yl)ethyl)piperidin-1-yl)carbamoyl)phenyl)amino)-5-fluoropyrimidin-4-yl)amino)benzamide (Compound 96)

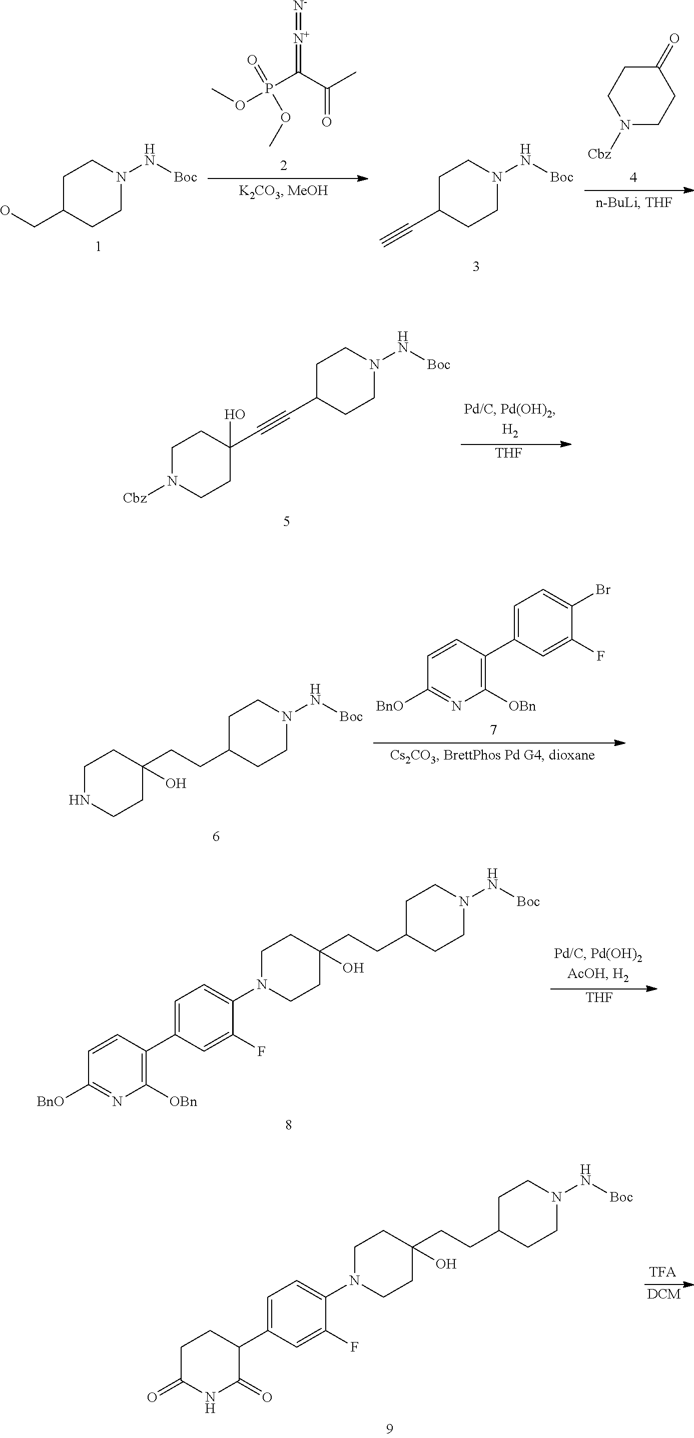

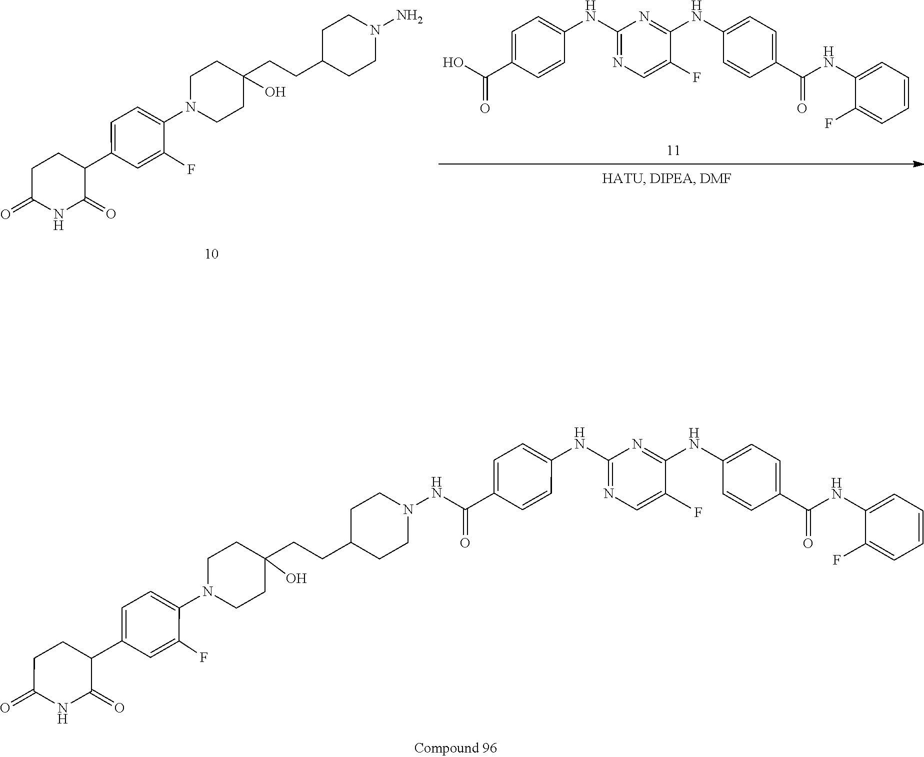 [SMILES: NN1CCC(CCC2(O)CCN(c3ccc(C4CCC(=O)NC4=O)cc3F)CC2)CC1 ; O=C(O)c1ccc(Nc2ncc(F)c(Nc3ccc(C(=O)Nc4ccccc4F)cc3)n2)cc1 ; O=C1CCC(c2ccc(N3CCC(O)(CCC4CCN(NC(=O)c5ccc(Nc6ncc(F)c(Nc7ccc(C(=O)Nc8ccccc8F)cc7)n6)cc5)CC4)CC3)c(F)c2)C(=O)N1]

### Step 1. Synthesis of tert-butyl (4-ethynylpiperidin-1-yl)carbamate (3)

To a solution of tert-butyl (4-formylpiperidin-1-yl)carbamate (1.8 g, 7.88 mmol) in MeOH (40 mL) was added K₂CO₃ (3.27 g, 23.65 mmol) and dimethyl (1-diazo-2-oxopropyl)phosphonate (3.79 g, 19.71 mmol) at 0° C. The mixture was stirred at 25° C. for 1 hr. TLC (petroleum ether:EtOAc=3:1) indicated tert-butyl (4-formylpiperidin-1-yl)carbamate was consumed completely and one new spot formed. The reaction mixture was diluted with water (30 mL) and extracted with EtOAc (30 mL×3). The combined organic layers were washed with brine (30 mL), dried over Na₂SO₄, filtered and concentrated under reduced pressure. The residue was purified by flash silica gel chromatography (ISCO®; 40 g SepaFlash® Silica Flash Column, Eluent of 15-25% EtOAc/Petroleum ether gradient @ 100 mL/min) to afford tert-butyl (4-ethynylpiperidin-1-yl)carbamate (1.1 g, 4.90 mmol, 62.20% yield) as a yellow solid.

¹H NMR (400 MHz, CDCl₃) δ=5.47 (br s, 1H), 3.02-2.87 (m, 2H), 2.75-2.59 (m, 2H), 2.44 (br s, 1H), 2.08 (d, J=2.3 Hz, 1H), 1.97-1.89 (m, 2H), 1.84-1.75 (m, 2H), 1.45 (s, 9H).

### Step 2. Synthesis of benzyl 4-((1-((tert-butoxycarbonyl)amino)piperidin-4-yl)ethynyl)-4-hydroxypiperidine-1-carboxylate (5)

To a solution of tert-butyl (4-ethynylpiperidin-1-yl)carbamate (1.1 g, 4.90 mmol) in THF (15 mL) was added n-BuLi (2.5 M, 5.89 mL) at −78° C. under N₂ atmosphere. After stirring at −78° C. for 0.5 hr, a solution of benzyl 4-oxopiperidine-1-carboxylate (1.14 g, 4.90 mmol, 976.11 μL) in THF (15 mL) was added and the resulting mixture was stirred at −78° C. for 1.5 hr under N₂ atmosphere. TLC (petroleum ether:EtOAc=1:1) showed part of tert-butyl (4-ethynylpiperidin-1-yl)carbamate was remained and one major new spot with larger polarity was detected. The reaction mixture was quenched by addition NH₄Cl (30 mL) at 0° C., and then diluted with water (20 mL) and extracted with EtOAc (30 mL×3). The combined organic layers were washed with water (20 mL), dried over Na₂SO₄, filtered and concentrated under reduced pressure. The residue was purified by flash silica gel chromatography (ISCO®; 40 g SepaFlash® Silica Flash Column, Eluent of 20~50% EtOAc/Petroleum ether gradient @ 80 mL/min) to afford benzyl 4-((1-((tert-butoxycarbonyl)amino)piperidin-4-yl)ethynyl)-4-hydroxypiperidine-1-carboxylate (1.1 g, 2.40 mmol, 49.02% yield) as a yellow oil.

¹H NMR (400 MHz, CDCl₃) δ=7.39-7.35 (m, 4H), 7.34-7.30 (m, 1H), 5.50 (br s, 1H), 5.14 (s, 2H), 3.97-3.81 (m, 2H), 3.37-3.25 (m, 2H), 3.05-2.86 (m, 2H), 2.78-2.60 (m, 2H), 2.47 (br s, 1H), 2.27-2.12 (m, 1H), 1.98-1.82 (m, 4H), 1.82-1.69 (m, 4H), 1.45 (s, 9H).

### Step 3. Synthesis of tert-butyl (4-(2-(4-hydroxypiperidin-4-yl)ethyl)piperidin-1-yl)carbamate (6)

To a mixture of Pd/C (100 mg, 10% purity) and Pd(OH)₂ (100 mg, 20% purity) in THF (10 mL) was added benzyl 4-(2-(1-((tert-butoxycarbonyl)amino)piperidin-4-yl)ethyl)-4-hydroxypiperidine-1-carboxylate (0.93 g, 2.01 mmol) in THF (10 mL) under N₂ atmosphere. The reaction system was replaced three times by Ar and then three times by H₂. The mixture was stirred under H₂ atmosphere (50 Psi) at 25° C. for 16 hr. HNMR showed benzyl 4-(2-(1-((tert-butoxycarbonyl)amino)piperidin-4-yl)ethyl)-4-hydroxypiperidine-1-carboxylate was consumed completely. The mixture was diluted with THF (20 mL), filtered through a pad of celite, washed with THF (30 mL×3). The filtrate was concentrated under reduced pressure. The crude product was triturated with MTBE (10 mL) at 25° C. for 10 min to afford tert-butyl (4-(2-(4-hydroxypiperidin-4-yl)ethyl)piperidin-1-yl)carbamate (560 mg, 1.71 mmol, 84.88% yield) as a white solid. MS(M+H)⁺=328.5.

### Step 4. Synthesis of tert-butyl (4-(2-(1-(4-(2,6-bis(benzyloxy)pyridin-3-yl)-2-fluorophenyl)-4-hydroxypiperidin-4-yl) ethyl)piperidin-1-yl)carbamate (8)

To a mixture of tert-butyl (4-(2-(4-hydroxypiperidin-4-yl)ethyl)piperidin-1-yl)carbamate (440 mg, 1.34 mmol) and 2,6-bis(benzyloxy)-3-(4-bromo-3-fluorophenyl)pyridine (748.68 mg, 1.61 mmol) in dioxane (8 mL) was added Cs₂CO₃ (1.31 g, 4.03 mmol) and BrettPhos Pd G4 (123.69 mg, 134.37 μmol) under N₂ atmosphere. The mixture was stirred at 100° C. for 16 hrs under N₂ atmosphere. LCMS showed 2,6-bis(benzyloxy)-3-(4-bromo-3-fluorophenyl)pyridine was consumed completely and one main peak with desired mass was detected. The reaction mixture was diluted with water (20 mL) and extracted with EtOAc (30 mL×2). The combined organic layers were washed with brine (20 mL), dried over Na₂SO₄, filtered and concentrated under reduced pressure. The residue was purified by flash silica gel chromatography (ISCO®; 10 g SepaFlash® Silica Flash Column, Eluent of 25-35% EtOAc/Petroleum ether gradient @ 80 mL/min) to afford tert-butyl (4-(2-(1-(4-(2,6-bis(benzyloxy)pyridin-3-yl)-2-fluorophenyl)-4-hydroxypiperidin-4-yl) ethyl)piperidin-1-yl) carbamate (410 mg, 576.75 μmol, 42.92% yield) as a yellow solid. MS(M+H)⁺=711.4.

### Step 5. Synthesis of tert-butyl (4-(2-(1-(4-(2,6-dioxopiperidin-3-yl)-2-fluorophenyl)-4-hydroxypiperidin-4-yl)ethyl)piperidin-1-yl)carbamate (9)

To a mixture of Pd/C (40 mg, 10% purity) and Pd(OH)₂ (40 mg, 20% purity) in THF (5 mL) was added tert-butyl (4-(2-(1-(4-(2,6-bis(benzyloxy)pyridin-3-yl)-2-fluorophenyl)-4-hydroxypiperidin-4-yl) ethyl)piperidin-1-yl)carbamate (500 mg, 703.36 μmol) and AcOH (4.22 mg, 70.34 μmol, 4.03 μL) in THF (5 mL) under N₂ atmosphere. The reaction system was replaced three times by Ar and then three times by H₂. The mixture was stirred under H₂ atmosphere (50 Psi) at 25° C. for 32 hr. LCMS showed the starting material was consumed completely and one main peak with desired mass was detected. The mixture was diluted with THF (20 mL), filtered through a pad of celite, washed with THF (20 mL×3). The filtrate was concentrated under reduced pressure. The crude product was triturated with petroleum ether (10 mL) at 25° C. for 10 min to afford tert-butyl (4-(2-(1-(4-(2,6-dioxopiperidin-3-yl)-2-fluorophenyl)-4-hydroxypiperidin-4-yl)ethyl)piperidin-1-yl)carbamate (230 mg, 349.76 μmol, 49.73% yield, 81% purity) as a blue solid. MS(M+H)⁺=533.3.

### Step 6. Synthesis of 3-(4-(4-(2-(1-aminopiperidin-4-yl)ethyl)-4-hydroxypiperidin-1-yl)-3-fluorophenyl)piperidine-2,6-dione (10)

To a solution of tert-butyl (4-(2-(1-(4-(2,6-dioxopiperidin-3-yl)-2-fluorophenyl)-4-hydroxypiperidin-4-yl)ethyl)piperidin-1-yl)carbamate (210 mg, 319.35 μmol) in DCM (6 mL) was added TFA (3.07 g, 26.93 mmol, 2 mL). The mixture was stirred at 25° C. for 1 hr. LCMS showed the starting material was consumed completely and one main peak with desired mass was detected. The reaction mixture was concentrated under reduced pressure to afford 3-(4-(4-(2-(1-aminopiperidin-4-yl)ethyl)-4-hydroxypiperidin-1-yl)-3-fluorophenyl)piperidine-2,6-dione (175 mg, crude, TFA salt) as a yellow oil. MS(M+H)⁺=433.3.

### Step 7. Synthesis of N-(2-chlorophenyl)-4-((2-((4-((4-(2-(1-(4-(2,6-dioxopiperidin-3-yl)-2-fluorophenyl)-4-hydroxypiperidin-4-yl)ethyl)piperidin-1-yl)carbamoyl)phenyl)amino)-5-fluoropyrimidin-4-yl)amino)benzamide (Compound 96)

To a solution of 4-((4-((4-((2-chlorophenyl)carbamoyl)phenyl)amino)-5-fluoropyrimidin-2-yl)amino)benzoic acid (100 mg, 188.75 μmol) in DMF (2 mL) were added HATU (107.65 mg, 283.13 μmol), DIPEA (243.94 mg, 1.89 mmol, 328.77 μL) and 3-(4-(4-(2-(1-aminopiperidin-4-yl)ethyl)-4-hydroxypiperidin-1-yl)-3-fluorophenyl)piperidine-2,6-dione (154.75 mg, 283.13 μmol, TFA salt). The mixture was stirred at 25° C. for 1 hr. LCMS showed 3-(4-(4-(2-(1-aminopiperidin-4-yl)ethyl)-4-hydroxypiperidin-1-yl)-3-fluorophenyl)piperidine-2,6-dione was consumed completely and one main peak with desired mass. The reaction mixture was poured into water (10 mL) and filtered. The filter cake was collected and purified by prep-HPLC (column: Phenomenex luna C18 150*25 mm*10 μm; mobile phase: [water(TFA)-ACN]; gradient: 23%-53% B over 10 min) and lyophilized to afford N-(2-chlorophenyl)-4-((2-((4-((4-(2-(1-(4-(2,6-dioxopiperidin-3-yl)-2-fluorophenyl)-4-hydroxypiperidin-4-yl)ethyl)piperidin-1-yl)carbamoyl)phenyl)amino)-5-fluoropyrimidin-4-yl)amino)benzamide (109.5 mg, 78.85 μmol, 41.77% yield, 97.1% purity, 4TFA salt) as a white solid. MS(M+H)⁺=892.3.

¹H NMR (400 MHz, MeOD) δ=8.12 (d, J=4.2 Hz, 1H), 8.06-7.98 (m, 2H), 7.95-7.87 (m, 2H), 7.84-7.77 (m, 3H), 7.77-7.68 (m, 2H), 7.54 (dd, J=1.4, 8.0 Hz, 1H), 7.39 (dt, J=1.5, 7.7 Hz, 1H), 7.33-7.25 (m, 2H), 7.12-7.02 (m, 2H), 4.01 (dd, J=5.0, 12.1 Hz, 1H), 3.56-3.41 (m, 6H), 3.08-2.98 (m, 2H), 2.83-2.72 (m, 1H), 2.72-2.64 (m, 1H), 2.34-2.22 (m, 1H), 2.18-2.09 (m, 1H), 1.97-1.88 (m, 2H), 1.88-1.75 (m, 4H), 1.67-1.53 (m, 4H), 1.49-1.39 (m, 3H).

### Example 97. Synthesis of N-(2-chlorophenyl)-4-((2-((4-((2-((1-(4-(2,6-dioxopiperidin-3-yl)-2-fluorophenyl)piperidin-4-yl)methyl)-2,7-diazaspiro[3.5]nonan-7-yl)carbamoyl)phenyl) amino)-5-fluoropyrimidin-4-yl)amino)benzamide (Compound 97)

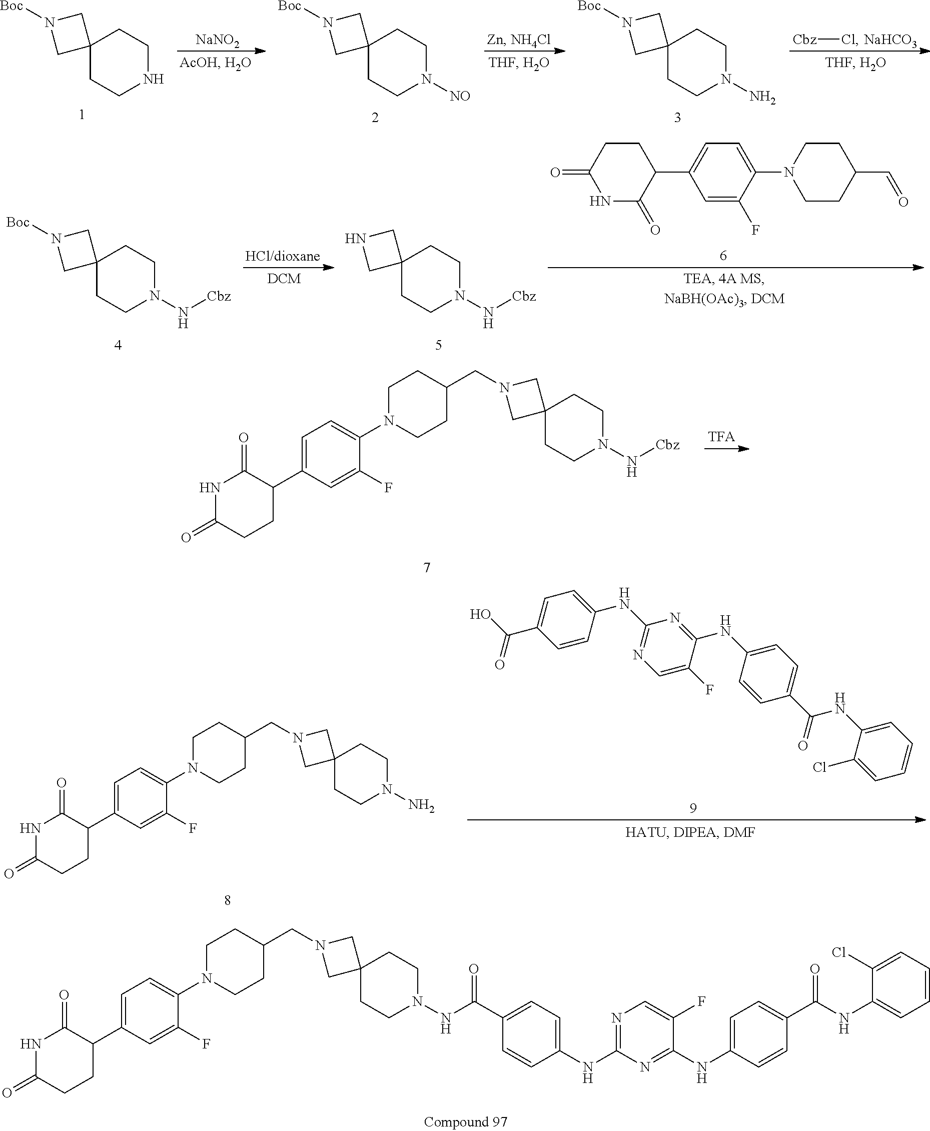

### Step 1. Synthesis of tert-butyl 7-nitroso-2,7-diazaspiro[3.5]nonane-2-carboxylate (2)

To a solution of tert-butyl 2,7-diazaspiro[3.5]nonane-2-carboxylate (3 g, 13.26 mmol) and NaNO₂ (2.74 g, 39.77 mmol) in H₂O (30 mL) was added AcOH (3.18 g, 53.02 mmol, 3.04 mL) dropwise at 0° C. The mixture was stirred at 20° C. for 16 hr. LCMS showed a peak (100%) with desired mass. To the reaction mixture was added aqueous NaHCO₃ solution to adjust pH=8. Then the mixture was extracted with EtOAc (50 mL×3). The combined organic phase was dried over Na₂SO₄ and concentrated under reduced pressure to afford tert-butyl 7-nitroso-2,7-diazaspiro[3.5]nonane-2-carboxylate (3 g, crude) as light yellow solid. MS(M−55)⁺=200.1.

### Step 2. Synthesis of tert-butyl 7-amino-2,7-diazaspiro[3.5]nonane-2-carboxylate (3)

To a solution of tert-butyl 7-nitroso-2,7-diazaspiro[3.5]nonane-2-carboxylate (3 g, 11.75 mmol) and NH₄Cl (3.77 g, 70.50 mmol) in THF (20 mL) and H₂O (10 mL) was added Zn (3.84 g, 58.75 mmol) portion-wise at 0° C. The mixture was warmed and stirred at 20° C. for 3 hr. LCMS showed starting material was consumed and desired mass was detected. The reaction mixture was filtered through celite pad and washed with 40 mL of THF. The filtrate was concentrated under reduced pressure to afford tert-butyl 7-amino-2,7-diazaspiro[3.5]nonane-2-carboxylate (6 g, crude) as white solid. MS(M+H)⁺=242.1.

### Step 3. Synthesis of tert-butyl 7-(benzyloxycarbonylamino)-2,7-diazaspiro[3.5]nonane-2-carboxylate (4)

To a solution of tert-butyl 7-amino-2,7-diazaspiro[3.5]nonane-2-carboxylate (6 g, 24.86 mmol) and NaHCO₃ (10.44 g, 124.31 mmol, 4.84 mL) in THF (60 mL) and H₂O (20 mL) was added CbzCl (7.63 g, 44.75 mmol, 6.39 mL) dropwise at 0° C. The mixture was stirred at 20° C. for 16 hr. LCMS showed a peak (13%) with desired mass. The reaction mixture was filtered. The filtrate was extracted with EtOAc (60 mL×2). The combined organic phase was dried over Na₂SO₄ and concentrated under reduced pressure. The residue was purified by flash silica gel chromatography (20 g SepaFlash® Silica Flash Column, Eluent of 0~50% EtOAc/Petroleum ether gradient @60 mL/min) to afford tert-butyl 7-(benzyloxycarbonylamino)-2,7-diazaspiro[3.5]nonane-2-carboxylate (900 mg, 2.23 mmol, 8.97% yield, 93% purity) as white solid. MS(M+H)⁺=376.2.

¹H NMR (400 MHz, CDCl₃-d) δ=7.41-7.30 (m, 5H), 5.73 (br s, 1H), 5.12 (br s, 2H), 3.62 (s, 4H), 2.80-2.65 (m, 4H), 1.90-1.80 (m, 4H), 1.44 (s, 9H).

### Step 4. Synthesis of benzyl N-(2,7-diazaspiro[3.5]nonan-7-yl)carbamate (5)

To a solution of tert-butyl 7-(benzyloxycarbonylamino)-2,7-diazaspiro[3.5]nonane-2-carboxylate (450 mg, 1.20 mmol) in DCM (5 mL) was added HCl/dioxane (2 M, 1 mL). The mixture was stirred at 20° C. for 3 hr. LCMS showed starting material was nearly consumed. The reaction mixture was concentrated under reduced pressure to afford benzyl N-(2,7-diazaspiro[3.5]nonan-7-yl) carbamate (300 mg, 937.01 μmol, 78.18% yield, 86% purity, HCl) as a white solid. MS(M+H)⁺=276.1.

### Step 5. Synthesis of benzyl N-[2-[[1-[4-(2,6-dioxo-3-piperidyl)-2-fluoro-phenyl]-4-piperidyl]methyl]-2,7-diazaspiro[3.5]nonan-7-yl]carbamate (7)

To a solution of 1-[4-(2,6-dioxo-3-piperidyl)-2-fluoro-phenyl]piperidine-4-carbaldehyde (306.29 mg, 962.14 μmol) and benzyl N-(2,7-diazaspiro[3.5]nonan-7-yl) carbamate (300 mg, 962.14 μmol, HCl) in DCM (5 mL) were added TEA (292.07 mg, 2.89 mmol, 401.75 μL) and 4 Å MS (100 mg, 962.14 μmol). After stirring at 20° C. for 0.5 hr, NaBH(OAc)₃ (611.75 mg, 2.89 mmol) was added and the resulting mixture was stirred at 20° C. for 16 hr. LCMS showed a peak (81%) with desired mass. The reaction mixture was filtered and the filtrate was diluted with 30 mL of aqueous NH₄Cl solution and extracted with DCM (30 mL×2). The combined organic phase was dried over Na₂SO₄ and concentrated under reduced pressure to afford benzyl N-[2-[[1-[4-(2,6-dioxo-3-piperidyl)-2-fluoro-phenyl]-4-piperidyl]methyl]-2,7-diazaspiro[3.5]nonan-7-yl]carbamate (600 mg, 893.22 μmol, 92.84% yield, 86% purity) as yellow oil. MS(M+H)⁺=578.3.

### Step 6. Synthesis of 3-[4-[4-[(7-amino-2,7-diazaspiro[3.5]nonan-2-yl)methyl]-1-piperidyl]-3-fluoro-phenyl]piperidine-2,6-dione (8)

A mixture of benzyl N-[2-[[1-[4-(2,6-dioxo-3-piperidyl)-2-fluoro-phenyl]-4-piperidyl]methyl]-2,7-diazaspiro[3.5]nonan-7-yl]carbamate (200 mg, 346.21 μmol) in TFA (2 mL) was stirred at 60° C. for 3.5 hr. LCMS showed a peak (58%) with desired mass and a peak (4%) with starting material. The reaction mixture was concentrated under reduced pressure to afford 3-[4-[4-[(7-amino-2,7-diazaspiro[3.5]nonan-2-yl)methyl]-1-piperidyl]-3-fluoro-phenyl]piperidine-2,6-dione (200 mg, crude, TFA) as yellow oil. MS(M+H)⁺=444.3.

### Step 7. Synthesis of N-(2-chlorophenyl)-4-((2-((4-((2-((1-(4-(2,6-dioxopiperidin-3-yl)-2-fluorophenyl)piperidin-4-yl)methyl)-2,7-diazaspiro[3.5]nonan-7-yl)carbamoyl)phenyl) amino)-5-fluoropyrimidin-4-yl)amino)benzamide (Compound 97)

To a solution of 3-[4-[4-[(7-amino-2,7-diazaspiro[3.5]nonan-2-yl)methyl]-1-piperidyl]-3-fluoro-phenyl]piperidine-2,6-dione (200 mg, 208.04 μmol, TFA) and 4-[[4-[4-[(2-chlorophenyl) carbamoyl]anilino]-5-fluoro-pyrimidin-2-yl]amino]benzoic acid (99.42 mg, 208.04 mol) in DMF (3 mL) was added HATU (94.92 mg, 249.65 μmol) and DIPEA (134.44 mg, 1.04 mmol, 181.19 μL). The mixture was stirred at 20° C. for 16 hr. LCMS showed a peak (22%) with desired mass. The reaction mixture was diluted with 30 mL of water and filtered. The filter cake was collected and purified by prep-HPLC (column: Phenomenex luna C18 150*25 mm*10 um; mobile phase: [water (TFA)-ACN]; gradient: 22%-52% B over 10 min) and lyophilized to afford N-(2-chlorophenyl)-4-((2-((4-((2-((1-(4-(2,6-dioxopiperidin-3-yl)-2-fluorophenyl)piperidin-4-yl)methyl)-2,7-diazaspiro[3.5]nonan-7-yl)carbamoyl)phenyl)amino)-5-fluoropyrimidin-4-yl)amino)benzamide (31.8 mg, 27.54 μmol, 13.24% yield, 98% purity, 2TFA) as white solid. MS(M+H)⁺=903.3.

¹H NMR (400 MHz, DMSO-d₆) δ=10.81 (s, 1H), 9.95 (s, 2H), 9.83-9.61 (m, 2H), 9.59-9.38 (m, 1H), 8.25 (d, J=3.3 Hz, 1H), 8.05-8.00 (m, 4H), 7.84-7.67 (m, 4H), 7.65 (br d, J=7.8 Hz, 1H), 7.57 (br d, J=8.0 Hz, 1H), 7.40 (br t, J=7.6 Hz, 1H), 7.33-7.25 (m, 1H), 7.08-6.89 (m, 3H), 4.11-3.98 (m, 2H), 3.95-3.85 (m, 2H), 3.80 (br dd, J=4.9, 11.9 Hz, 1H), 3.37-3.33 (m, 2H), 3.25-3.15 (m, 2H), 2.98-2.79 (m, 4H), 2.69-2.59 (m, 3H), 2.27-2.12 (m, 1H), 2.07-1.67 (m, 8H), 1.48-1.31 (m, 2H).

### Example 98. Synthesis of N-(2-chlorophenyl)-6-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino) nicotinamide (Compound 98)

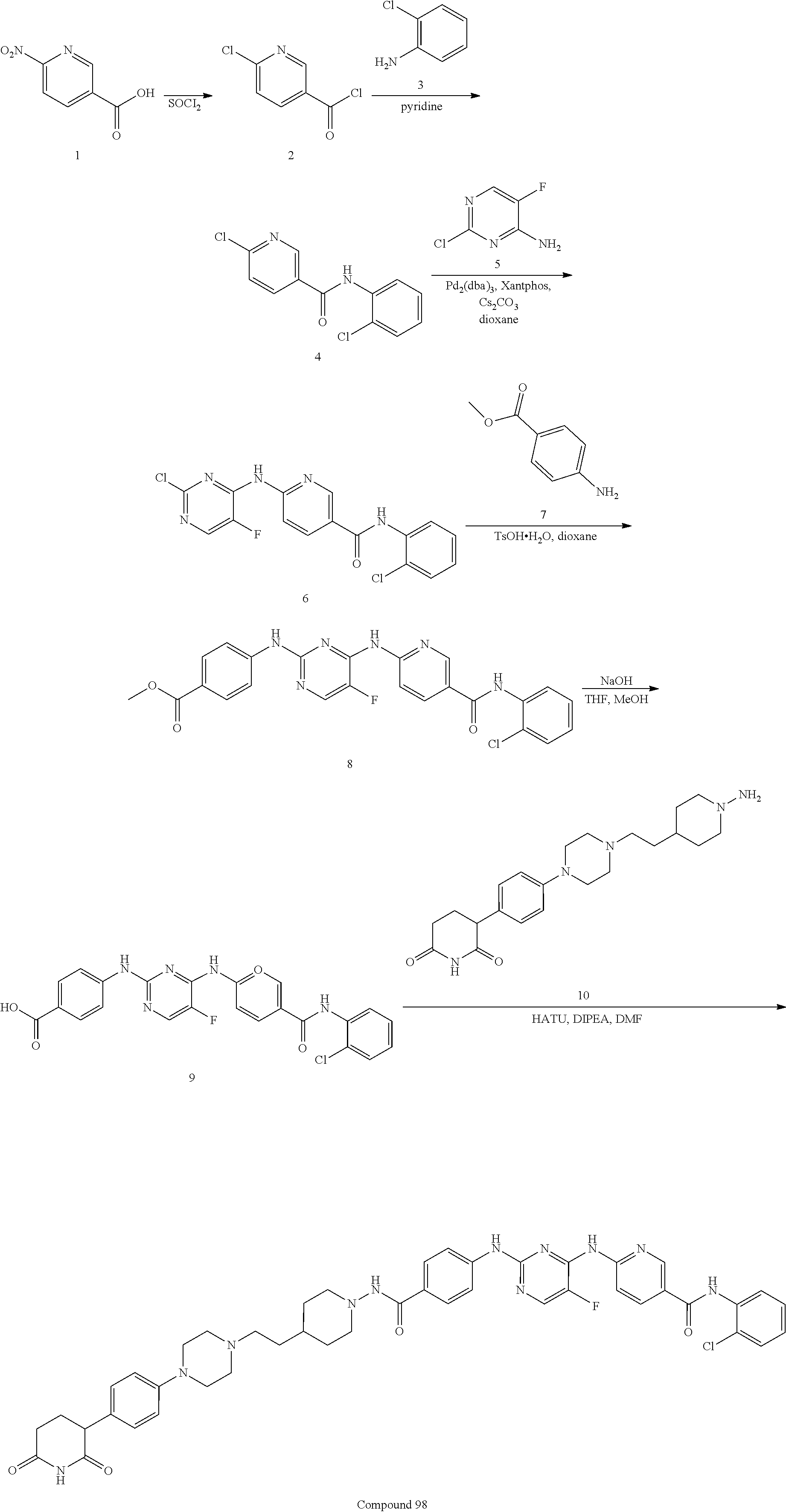

### Step 1. Synthesis of 6-chloronicotinoyl chloride (2)

A solution of 6-nitronicotinic acid (2 g, 11.90 mmol) in SOCl₂ (10 mL) was stirred at 75° C. for 1.5 hr. TLC (petroleum ether:EtOAc=3:1) indicated 6-nitronicotinic acid was consumed completely and two new spot formed. The reaction mixture was concentrated under reduced pressure to afford 6-chloronicotinoyl chloride (2.22 g, 11.90 mmol, 100.00% yield, crude) as a yellow solid, which was used for the next step directly.

### Step 2. Synthesis of 6-chloro-N-(2-chlorophenyl)nicotinamide (4)

To a solution of 2-chloroaniline (1.52 g, 11.90 mmol, 1.25 mL) in pyridine (22 mL) was added 6-chloronicotinoyl chloride (2.22 g, 11.90 mmol). The mixture was stirred at 25° C. for 4 hr. LCMS showed ~16% of 2-chloroaniline remained and ~42% of desired compound was detected. The mixture was diluted with water (50 mL) and extracted with EtOAc (30 mL×3). The combined organic layers were washed with water (50 mL), dried over Na₂SO₄, filtered and concentrated under reduced pressure. The residue was purified by flash silica gel chromatography (ISCO®; 80 g SepaFlash® Silica Flash Column, Eluent of 15-30% EtOAc/Petroleum ether gradient @ 100 mL/min) to afford 6-chloro-N-(2-chlorophenyl)nicotinamide (940 mg, 3.52 mmol, 29.57% yield) as a yellow solid. MS(M+H)⁺=267.1.

### Step 3. Synthesis of 6-((2-chloro-5-fluoropyrimidin-4-yl)amino)-N-(2-chlorophenyl)nicotinamide (6)

To a solution of 6-chloro-N-(2-chlorophenyl)nicotinamide (940 mg, 3.52 mmol) and 2-chloro-5-fluoropyrimidin-4-amine (519.21 mg, 3.52 mmol) in dioxane (10 mL) was added Cs₂CO₃ (3.44 g, 10.56 mmol) and Pd₂(dba)₃ (322.26 mg, 351.91 μmol) and Xantphos (203.62 mg, 351.91 μmol). The mixture was stirred at 100° C. for 16 hr under N₂ atmosphere. LCMS showed 6-chloro-N-(2-chlorophenyl)nicotinamide was consumed completely and one main peak with desired mass was detected. The reaction mixture was filtered and the filtrate was concentrated under reduced pressure to remove solvent. The residue was purified by flash silica gel chromatography (ISCO®; 40 g SepaFlash® Silica Flash Column, Eluent of 30-100% EtOAc/Petroleum ether, gradient @ 100 mL/min) to afford 6-((2-chloro-5-fluoropyrimidin-4-yl)amino)-N-(2-chlorophenyl)nicotinamide (1.1 g, 2.91 mmol, 82.65% yield) as a yellow solid.

¹H NMR (400 MHz, DMSO-d₆) δ=10.85 (s, 1H), 10.20 (s, 1H), 8.97 (d, J=2.0 Hz, 1H), 8.50 (d, J=3.2 Hz, 1H), 8.40 (dd, J=2.4, 8.7 Hz, 1H), 8.10 (d, J=8.7 Hz, 1H), 7.61 (dd, J=1.5, 7.8 Hz, 1H), 7.58 (dd, J=1.3, 8.1 Hz, 1H), 7.41 (dt, J=1.4, 7.7 Hz, 1H), 7.32 (dt, J=1.6, 7.7 Hz, 1H).

### Step 4. Synthesis of methyl 4-((4-((5-((2-chlorophenyl)carbamoyl)pyridin-2-yl)amino)-5-fluoropyrimidin-2-yl)amino)benzoate (8)

To a solution of 6-((2-chloro-5-fluoropyrimidin-4-yl)amino)-N-(2-chlorophenyl)nicotinamide (0.6 g, 1.59 mmol) and methyl 4-aminobenzoate (239.82 mg, 1.59 mmol) in dioxane (6 mL) was added TsOH·H₂O (905.35 mg, 4.76 mmol). The mixture was stirred at 100° C. for 16 hr. LCMS showed methyl 4-aminobenzoate was consumed completely and one main peak with desired mass was detected. The mixture was treated with Na₂CO₃ solution to adjust pH>7, the suspension was filtered. The filter cake was triturated with (MTBE/EtOAc=1/1, 10 mL) at 25° C. for 30 min to afford methyl 4-((4-((5-((2-chlorophenyl)carbamoyl)pyridin-2-yl)amino)-5-fluoropyrimidin-2-yl)amino)benzoate (770 mg, 1.56 mmol, 98.47% yield) as a light yellow solid. MS(M+H)⁺=493.1.

### Step 5. Synthesis of 4-((4-((5-((2-chlorophenyl)carbamoyl)pyridin-2-yl)amino)-5-fluoropyrimidin-2-yl)amino)benzoic acid (9)

To a solution of methyl 4-((4-((5-((2-chlorophenyl)carbamoyl)pyridin-2-yl)amino)-5-fluoropyrimidin-2-yl)amino)benzoate (770 mg, 1.56 mmol) in THF (10.4 mL) and MeOH (2.6 mL) was added NaOH (3 M, 2.60 mL). The mixture was stirred at 40° C. for 16 hr. LCMS showed the starting material was consumed completely and one main peak with desired mass. The mixture was neutralized to pH=7 by HCl solution (6 M). The suspension was filtered and washed with MeOH (30 mL). The filter cake was collected and dried to afford 4-((4-((5-((2-chlorophenyl)carbamoyl)pyridin-2-yl)amino)-5-fluoropyrimidin-2-yl)amino)benzoic acid (510 mg, 1.07 mmol, 68.17% yield) as a light yellow solid. MS(M+H)⁺=479.1.

### Step 6. Synthesis of N-(2-chlorophenyl)-6-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)nicotinamide (Compound 98)

To a solution of 4-((4-((5-((2-chlorophenyl)carbamoyl)pyridin-2-yl)amino)-5-fluoropyrimidin-2-yl)amino)benzoic acid (150 mg, 313.24 μmol) in DMF (2 mL) were added HATU (178.66 mg, 469.86 μmol), DIPEA (202.42 mg, 1.57 mmol, 272.80 μL) and 3-(4-(4-(2-(1-aminopiperidin-4-yl)ethyl)piperazin-1-yl)phenyl)piperidine-2,6-dione (204.86 mg, 469.86 μmol, HCl salt). The mixture was stirred at 25° C. for 1 hr. LCMS showed 3-(4-(4-(2-(1-aminopiperidin-4-yl)ethyl)piperazin-1-yl)phenyl)piperidine-2,6-dione was consumed completely and one main peak with desired mass was detected. The reaction mixture was poured into water (10 mL) and filtered. The filter cake was collected to afford the crude product, which was purified by prep-HPLC (column: Phenomenex luna C18 150*25 mm*10 m; mobile phase: [water(TFA)-ACN]; gradient: 23%-53% B over 10 min) and lyophilized to afford N-(2-chlorophenyl)-6-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)amino)nicotinamide (92.4 mg, 73.62 μmol, 23.50% yield, 95.8% purity, 3TFA salt) as a white solid. MS(M+H)⁺=860.3.

¹H NMR (400 MHz, CD₃OD) δ=8.95 (d, J=1.7 Hz, 1H), 8.53-8.45 (m, 1H), 8.44-8.36 (m, 1H), 8.22 (d, J=3.3 Hz, 1H), 7.85-7.77 (m, 4H), 7.75 (dd, J=1.5, 8.0 Hz, 1H), 7.54 (dd, J=1.4, 8.0 Hz, 1H), 7.39 (dt, J=1.5, 7.8 Hz, 1H), 7.34-7.26 (m, 1H), 7.20 (d, J=8.8 Hz, 2H), 7.03 (d, J=8.8 Hz, 2H), 3.92-3.82 (m, 2H), 3.82-3.77 (m, 1H), 3.74-3.61 (m, 2H), 3.29-3.20 (m, 6H), 3.12-2.98 (m, 2H), 2.80-2.71 (m, 2H), 2.71-2.66 (m, 1H), 2.65-2.57 (m, 1H), 2.25-2.14 (m, 2H), 1.88-1.80 (m, 2H), 1.80-1.71 (m, 2H), 1.67-1.55 (m, 2H), 1.54-1.44 (m, 1H).

### Example 99. Synthesis of N-(2-chlorophenyl)-4-((2-((4-((1-(2-(4-(4-(2,6-dioxopiperidin-3-yl)phenyl)piperazin-1-yl)ethyl)piperidin-4-yl)methoxy)phenyl)amino)-5-fluoropyrimidin-4-yl)amino) benzamide (Compound 99)

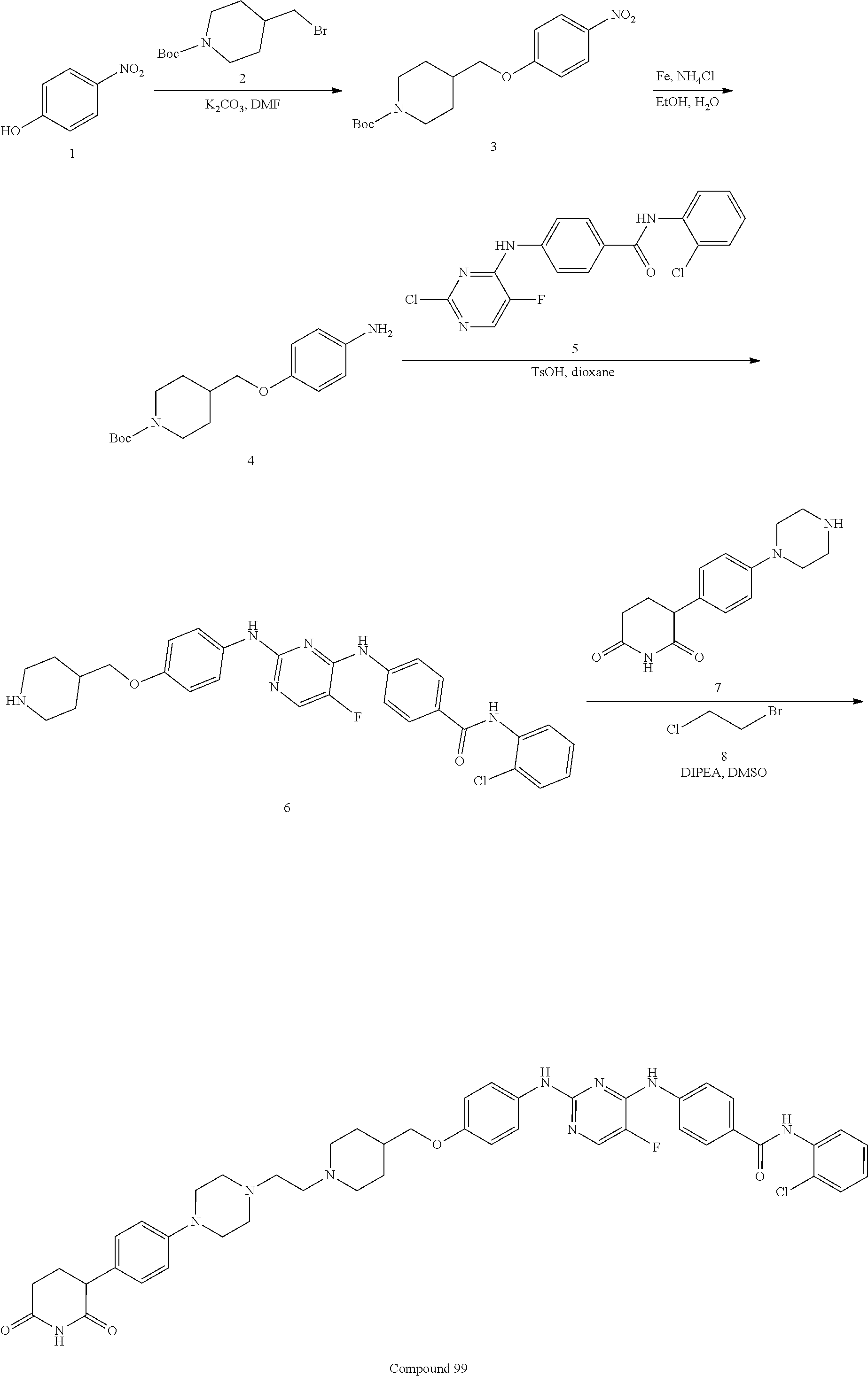

### Step 1. Synthesis of tert-butyl 4-(4-nitrophenoxy methyl)piperidine-1-carboxylate (3)

To a solution of 4-nitrophenol (1 g, 7.19 mmol) and tert-butyl 4-(bromomethyl)piperidine-1-carboxylate (2.20 g, 7.91 mmol) in DMF (10 mL) was added K₂CO₃ (2.98 g, 21.57 mmol). The mixture was stirred at 25° C. for 16 hr. LCMS showed 4-nitrophenol was consumed completely and desired mass was detected. The reaction mixture was diluted with H₂O (50 mL) and extracted with EtOAc (30 mL×3). The combined organic layers were washed with brine (100 mL), dried over Na₂SO₄, filtered and concentrated under reduced pressure. The residue was purified by flash silica gel chromatography (ISCO®; 20 g SepaFlash® Silica Flash Column, Eluent of 0~20% EtOAc/Petroleum ether, gradient @ 100 mL/min) to afford tert-butyl 4-((4-nitrophenoxy)methyl)piperidine-1-carboxylate (870 mg, 2.59 mmol, 35.98% yield) as a white solid. MS(M-Boc+H)+=236.2.

### Step 2. Synthesis of tert-butyl 4-((4-aminophenoxy)methyl)piperidine-1-carboxylate (4)

To a solution of tert-butyl 4-((4-nitrophenoxy)methyl)piperidine-1-carboxylate (870 mg, 2.59 mmol, 1 eq) in EtOH (18 mL) and H₂O (3.6 mL) were added Fe (722.17 mg, 12.93 mmol) and NH₄Cl (1.38 g, 25.86 mmol). The mixture was stirred at 80° C. for 1 hr. LCMS showed the starting material was consumed completely and desired mass. The reaction mixture was diluted with saturated Na₂CO₃ solution (50 mL) and extracted with EtOAc (30 mL×3). The combined organic layers were dried over Na₂ SO₄, filtered and concentrated under reduced pressure to afford tert-butyl 4-((4-aminophenoxy)methyl)piperidine-1-carboxylate (870 mg, crude) as a brown solid. MS(M−Boc+H)⁺=206.2.

### Step 3. Synthesis of N-(2-chlorophenyl)-4-((5-fluoro-2-((4-(piperidin-4-ylmethoxy)phenyl)amino)pyrimidin-4-yl)amino)benzamide (6)

To a solution of tert-butyl 4-((4-aminophenoxy)methyl)piperidine-1-carboxylate (292.43 mg, 954.40 μmol) and 4-((2-chloro-5-fluoropyrimidin-4-yl)amino)-N-(2-chlorophenyl)benzamide (400 mg, 1.06 mmol) in dioxane (4 mL) was added TsOH·H₂O (605.15 mg, 3.18 mmol). The mixture was stirred at 100° C. for 16 hr. LCMS showed tert-butyl 4-((4-aminophenoxy)methyl)piperidine-1-carboxylate was consumed completely and desired mass. The reaction mixture was diluted with H₂O (20 mL) and extracted with EtOAc (40 mL) and THF (40 mL). The combined organic layers were dried over Na₂ SO₄, filtered and concentrated under reduced pressure. The residue was purified by flash silica gel chromatography (ISCO®; 20 g SepaFlash® Silica Flash Column, Eluent of 0-30% MeOH/EtOAc @ 80 mL/min) to afford N-(2-chlorophenyl)-4-((5-fluoro-2-((4-(piperidin-4-ylmethoxy)phenyl)amino)pyrimidin-4-yl)amino)benzamide (110 mg, 201.09 μmol, 18.96% yield) as a white solid. MS(M+H)⁺=547.3.

### Step 4. Synthesis of N-(2-chlorophenyl)-4-((2-((4-((1-(2-(4-(4-(2,6-dioxopiperidin-3-yl)phenyl)piperazin-1-yl)ethyl)piperidin-4-yl)methoxy)phenyl)amino)-5-fluoropyrimidin-4-yl)amino) benzamide (Compound 99)

To a solution of N-(2-chlorophenyl)-4-((5-fluoro-2-((4-(piperidin-4-ylmethoxy)phenyl)amino)pyrimidin-4-yl)amino)benzamide (90 mg, 164.53 μmol) and 3-(4-(piperazin-1-yl)phenyl)piperidine-2,6-dione (50.97 mg, 164.53 μmol, HCl) and 1-bromo-2-chloroethane (235.95 mg, 1.65 mmol, 136.39 μL) in DMSO (2 mL) was added DIPEA (106.32 mg, 822.64 μmol, 143.29 μL). The mixture was stirred at 25° C. for 20 hr. LCMS showed N-(2-chlorophenyl)-4-((5-fluoro-2-((4-(piperidin-4-ylmethoxy)phenyl)amino)pyrimidin-4-yl)amino)benzamide was consumed completely and one main peak with desired mass. The reaction mixture was diluted with water (20 mL) and extracted with EtOAc (30 mL×3). The combined organic layers were washed with brine (30 mL×2), dried over Na₂SO₄, filtered and concentrated under reduced pressure. The residue was purified by prep-TLC (SiO₂, EtOAc:MeOH=8:1). The residue was purified by prep-HPLC (column: Phenomenex luna C18 150×25 mm×10 μm; mobile phase: [water(TFA)-ACN]; gradient: 18%-48% B over 10 min) and lyophilized to afford N-(2-chlorophenyl)-4-((2-((4-((1-(2-(4-(4-(2,6-dioxopiperidin-3-yl)phenyl)piperazin-1-yl)ethyl)piperidin-4-yl)methoxy)phenyl)amino)-5-fluoropyrimidin-4-yl)amino)benzamide (4.7 mg, 3.79 μmol, 2.31% yield, 95.9% purity, 3TFA) as a yellow solid. MS(M+H)⁺=846.4.

¹H NMR (400 MHz, DMSO-d₆) δ=10.79 (s, 1H), 9.93 (s, 1H), 9.72 (s, 1H), 9.24 (s, 1H), 8.16 (d, J=3.8 Hz, 1H), 8.04-7.93 (m, 4H), 7.66-7.61 (m, 1H), 7.59-7.50 (m, 3H), 7.40 (dt, J=1.3, 7.6 Hz, 1H), 7.32-7.25 (m, 1H), 7.11 (d, J=7.0 Hz, 2H), 7.02-6.96 (m, 2H), 6.90 (br d, J=9.0 Hz, 2H), 3.79-3.74 (m, 5H), 3.59-3.41 (m, 6H), 3.39-2.92 (m, 8H), 2.68-2.60 (m, 1H), 2.47-2.43 (m, 1H), 2.21-2.12 (m, 1H), 2.10-1.92 (m, 4H), 1.76-1.54 (m, 2H).

### Example 100. Synthesis of N-(2-chlorophenyl)-4-((2-((4-(((1-(2-(4-(4-(2,6-dioxopiperidin-3-yl)phenyl)piperazin-1-yl)ethyl)piperidin-4-yl)(methyl)amino)methyl)phenyl)amino)-5-fluoropyrimidin-4-yl)amino)benzamide (Compound 100)

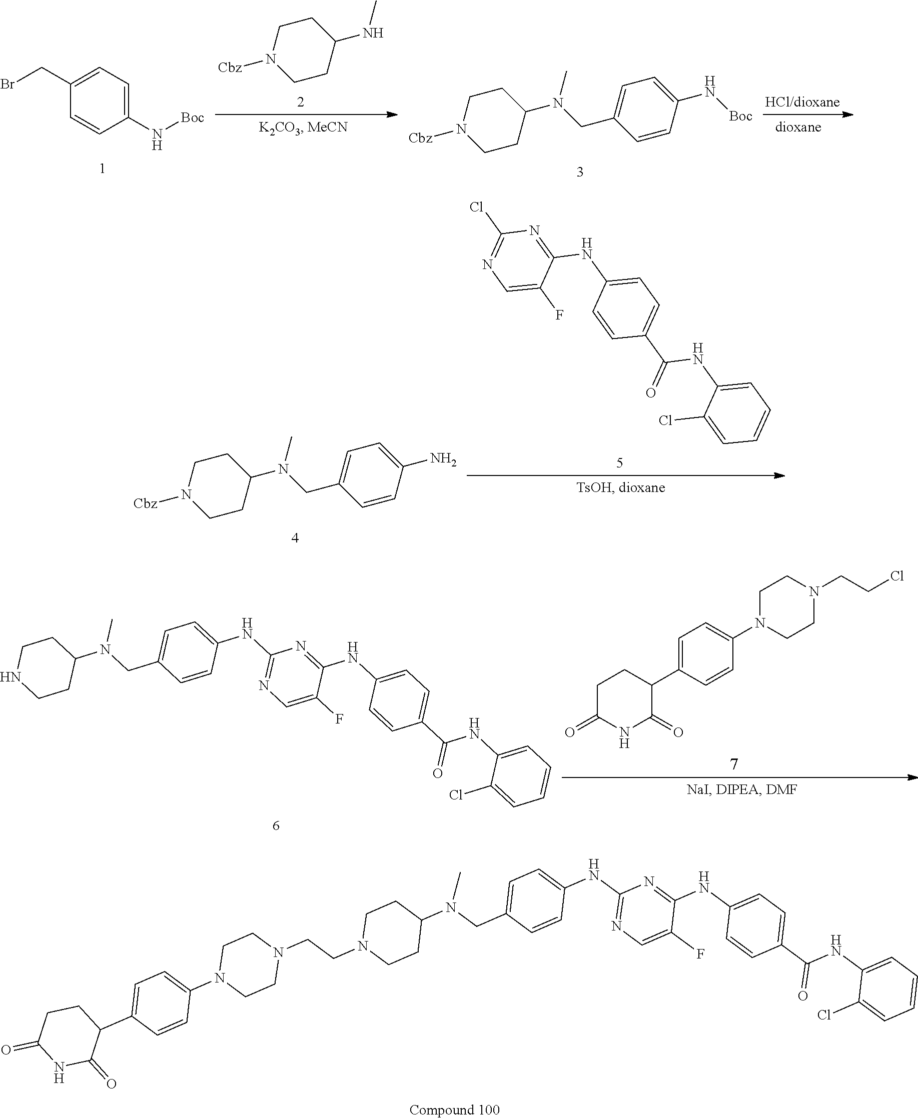

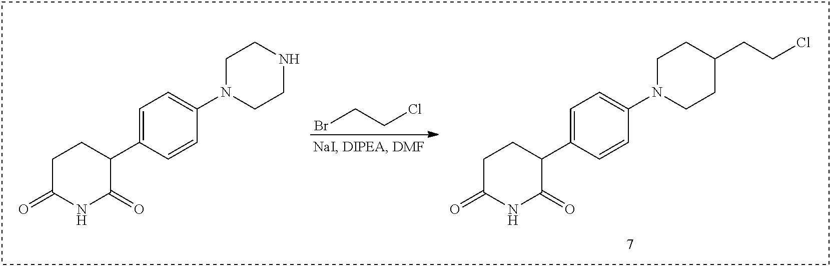

### Step 1. Synthesis of benzyl 4-((4-((tert-butoxycarbonyl)amino)benzyl)(methyl)amino)piperidine-1-carboxylate (3)

To a solution of tert-butyl (4-(bromomethyl)phenyl)carbamate (1 g, 3.49 mmol) and benzyl 4-(methylamino)piperidine-1-carboxylate (867.75 mg, 3.49 mmol) in MeCN (15 mL) was added K₂CO₃ (965.92 mg, 6.99 mmol). The mixture was stirred at 25° C. for 16 hr. LCMS showed one main peak with desired mass. The reaction mixture was diluted with water (40 mL) and extracted with EtOAc (20 mL×3). The combined organic layers were washed with brine (20 mL×2), dried over Na₂SO₄, filtered and concentrated under reduced pressure. The residue was purified by flash silica gel chromatography (ISCO®; 40 g SepaFlash® Silica Flash Column, Eluent of 40~50% EtOAc/Petroleum ether, gradient @ 80 mL/min) to afford benzyl 4-((4-((tert-butoxycarbonyl)amino)benzyl)(methyl)amino)piperidine-1-carboxylate (1.12 g, 2.47 mmol, 70.66% yield) as a white solid. MS(M+H)⁺=454.3.

### Step 2. Synthesis of benzyl 4-((4-aminobenzyl)(methyl)amino)piperidine-1-carboxylate (4)

A mixture of benzyl 4-((4-((tert-butoxycarbonyl)amino)benzyl)(methyl)amino)piperidine-1-carboxylate (1.12 g, 2.47 mmol) in dioxane (5 mL) in HCl/dioxane (2 M, 15 mL) was stirred at 25° C. for 3 hr. LCMS showed a 85% peak with desired mass. The reaction mixture was concentrated under reduced pressure to afford benzyl 4-((4-aminobenzyl)(methyl)amino)piperidine-1-carboxylate (1.1 g, crude, HCl) as a yellow solid. MS(M+H)⁺=354.2.

### Step 3. Synthesis of N-(2-chlorophenyl)-4-((5-fluoro-2-((4-((methyl(piperidin-4-yl)amino)methyl)phenyl)amino)pyrimidin-4-yl)amino)benzamide (6)

To a solution of benzyl 4-((4-aminobenzyl)(methyl)amino)piperidine-1-carboxylate (1.1 g, 2.82 mmol, HCl) and 4-((2-chloro-5-fluoropyrimidin-4-yl)amino)-N-(2-chlorophenyl)benzamide (1.06 g, 2.82 mmol) in dioxane (11 mL) was added TsOH·H₂O (1.61 g, 8.46 mmol). The mixture was stirred at 100° C. for 16 hr. LCMS showed benzyl 4-((4-aminobenzyl)(methyl)amino)piperidine-1-carboxylate was consumed completely and ~34% peak of desired mass. The mixture was treated with Na₂CO₃ solution to adjust pH=7 and filtered. The filtrate was extracted with EtOAc (20 mL×3). The filter cake was washed with EtOAc (30 mL). The combined organic layers were dried over Na₂SO₄, filtered and concentrated in vacuum. The residue was purified by flash silica gel chromatography (ISCO®; 20 g SepaFlash® Silica Flash Column, Eluent of 0-10% MeOH in EtOAc with 5% NH₃—H₂O), gradient @ 80 mL/min) to afford N-(2-chlorophenyl)-4-((5-fluoro-2-((4-((methyl(piperidin-4-yl)amino)methyl)phenyl)amino)pyrimidin-4-yl)amino) benzamide (100 mg, 178.55 μmol, 6.33% yield) as a yellow solid. MS(M+H)⁺=560.1.

¹H NMR (400 MHz, DMSO-d₆) δ=9.90 (br s, 1H), 9.64 (s, 1H), 9.26 (s, 1H), 8.18 (d, J=3.5 Hz, 1H), 8.05-8.00 (m, 2H), 8.00-7.93 (m, 2H), 7.65 (dd, J=1.4, 7.9 Hz, 1H), 7.60 (d, J=8.6 Hz, 2H), 7.57 (dd, J=1.4, 8.0 Hz, 1H), 7.40 (dt, J=1.3, 7.6 Hz, 1H), 7.29 (dt, J=1.6, 7.7 Hz, 1H), 7.17 (d, J=8.4 Hz, 2H), 3.00 (br d, J=11.5 Hz, 2H), 2.48-2.38 (m, 5H), 2.09 (s, 3H), 1.73-1.66 (m, 2H), 1.45-1.36 (m, 2H).

### Step 4. Synthesis of 3-(4-(4-(2-chloroethyl)piperazin-1-yl)phenyl)piperidine-2,6-dione (7)

To a solution of 3-(4-(piperazin-1-yl)phenyl)piperidine-2,6-dione (200 mg, 645.60 mol, HCl) in DMF (2 mL) was added NaI (9.68 mg, 64.56 μmol) and DIPEA (250.31 mg, 1.94 mmol, 337.35 μL) and 1-bromo-2-chloroethane (1.85 g, 12.91 mmol, 1.07 mL). The mixture was stirred at 80° C. for 0.5 hr. LCMS showed 3-(4-(piperazin-1-yl)phenyl)piperidine-2,6-dione was consumed completely and one main peak with desired mass. The reaction mixture was diluted with water (20 mL) and extracted with EtOAc (20 mL×3). The combined organic layers were washed with brine (20 mL), dried over Na₂SO₄, filtered and concentrated under reduced pressure. The crude product was triturated with MTBE (10 mL) at 25° C. for 30 min to afford 3-(4-(4-(2-chloroethyl)piperazin-1-yl)phenyl)piperidine-2,6-dione (120 mg, 285.86 μmol, 44.28% yield, 80% purity) as a yellow solid. MS(M+H)⁺=336.1.

### Step 5. Synthesis of N-(2-chlorophenyl)-4-((2-((4-(((1-(2-(4-(4-(2,6-dioxopiperidin-3-yl)phenyl)piperazin-1-yl)ethyl)piperidin-4-yl)(methyl)amino)methyl)phenyl)amino)-5-fluoropyrimidin-4-yl)amino)benzamide (Compound 100)

To a solution of N-(2-chlorophenyl)-4-((5-fluoro-2-((4-((methyl(piperidin-4-yl)amino)methyl)phenyl)amino)pyrimidin-4-yl)amino)benzamide (80 mg, 142.84 μmol) in DMF (2 mL) were added NaI (2.14 mg, 14.28 μmol), DIPEA (55.38 mg, 428.52 μmol, 74.64 μL) and 3-(4-(4-(2-chloroethyl)piperazin-1-yl)phenyl)piperidine-2,6-dione (89.94 mg, 214.26 mol). The mixture was stirred at 80° C. for 1 hr. LCMS showed ~5% of N-(2-chlorophenyl)-4-((5-fluoro-2-((4-((methyl(piperidin-4-yl)amino)methyl)phenyl)amino)pyrimidin-4-yl)amino)benzamide remained and ~57% peak of desired mass. The reaction mixture was poured into water (10 mL) and filtered. The filter cake was collected and purified by prep-HPLC (column: Phenomenex luna C18 150×25 mm×10 um; mobile phase: [water(TFA)-ACN]; gradient: 11%-41% B over 12 min), the eluent was lyophilized to afford N-(2-chlorophenyl)-4-((2-((4-(((1-(2-(4-(4-(2,6-dioxopiperidin-3-yl)phenyl)piperazin-1-yl)ethyl)piperidin-4-yl)(methyl)amino)methyl)phenyl)amino)-5-fluoropyrimidin-4-yl) amino)benzamide (57.9 mg, 45.83 μmol, 32.08% yield, 95.1% purity, 3TFA) as a light yellow solid. MS(M+H)⁺=859.4.

¹H NMR (400 MHz, DMSO-d₆) δ=10.79 (s, 1H), 9.93 (s, 1H), 9.88-9.77 (m, 1H), 9.76 (s, 1H), 9.58 (s, 1H), 8.24 (d, J=3.5 Hz, 1H), 8.07-7.93 (m, 4H), 7.81 (d, J=8.7 Hz, 2H), 7.64 (dd, J=1.4, 7.9 Hz, 1H), 7.57 (dd, J=1.4, 8.0 Hz, 1H), 7.46-7.35 (m, 3H), 7.33-7.27 (m, 1H), 7.11 (d, J=8.7 Hz, 2H), 6.96 (d, J=8.8 Hz, 2H), 4.46-4.11 (m, 3H), 3.76 (dd, J=4.9, 11.1 Hz, 1H), 3.55-3.05 (m, 14H), 2.77-2.57 (m, 5H), 2.49-2.42 (m, 2H), 2.28-2.18 (m, 2H), 2.18-2.09 (m, 1H), 2.04-1.84 (m, 3H).

### Example 101. Synthesis of N-(2-chlorophenyl)-4-((2-((4-((4-(2-(4-(4-(2,6-dioxopiperidin-3-yl)phenyl) piperazin-1-yl)ethyl) piperidin-1-yl) carbamoyl)phenyl)amino)-5-fluoropyrimidin-4-yl)oxy) benzamide (Compound 101)

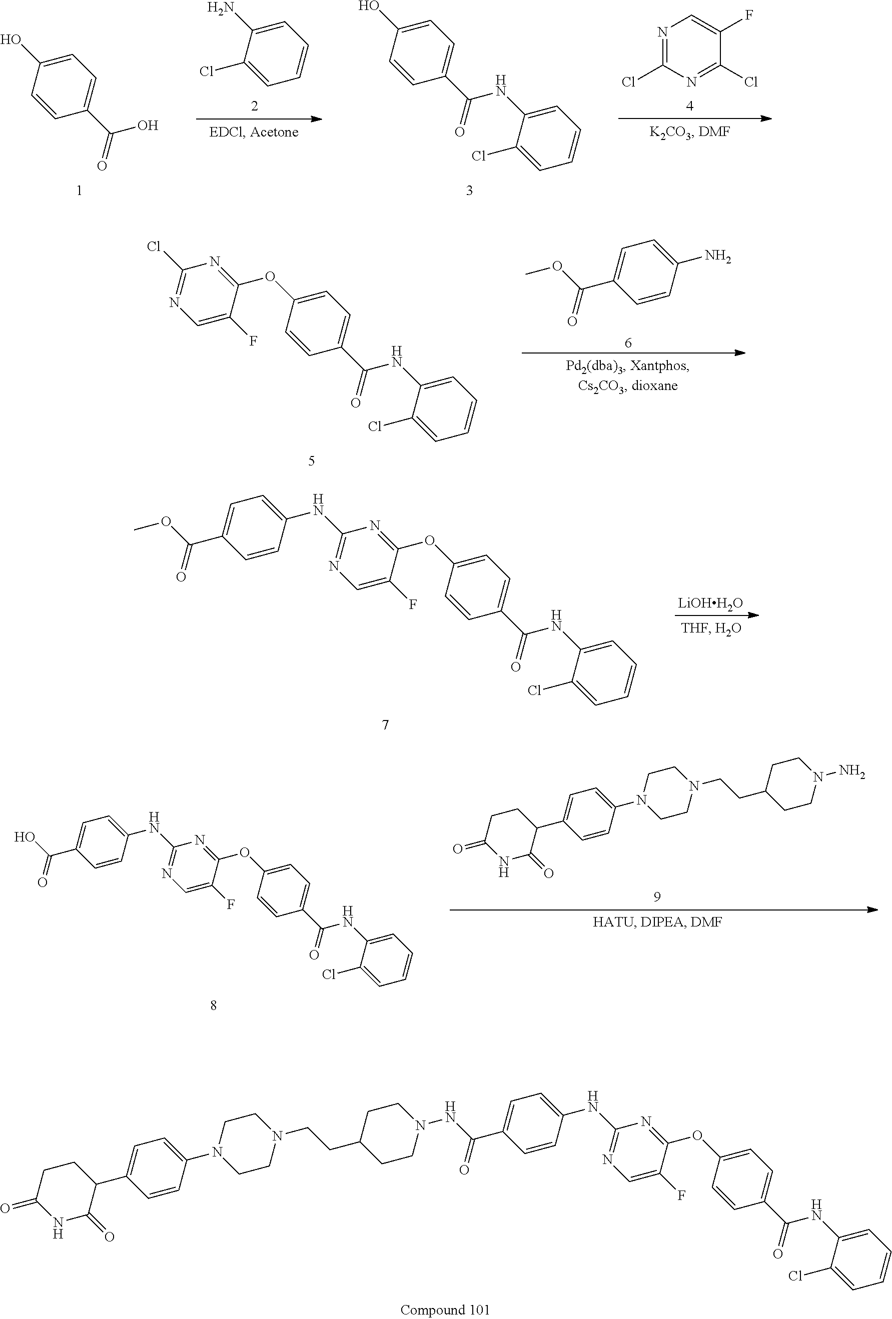

### Step 1. Synthesis of N-(2-chlorophenyl)-4-hydroxybenzamide (3)

To a solution of 4-hydroxybenzoic acid (2 g, 14.48 mmol) in acetone (10 mL) were added 2-chloroaniline (1.85 g, 14.48 mmol, 1.53 mL) and EDCI (3.33 g, 17.38 mmol). The mixture was stirred at 70° C. for 12 hr. LCMS showed 4-hydroxybenzoic acid was consumed completely and ~34% peak of desired mass. The reaction mixture was concentrated under reduced pressure to remove solvent. The residue was diluted with water (50 mL) and extracted with EtOAc (50 mL×3). The combined organic layers were washed with brine (30 mL), dried over Na₂SO₄, filtered and concentrated under reduced pressure. The residue was purified by flash silica gel chromatography (ISCO®; 40 g SepaFlash® Silica Flash Column, Eluent of 20-30% EtOAc/Petroleum ether, gradient @ 80 mL/min) to afford N-(2-chlorophenyl)-4-hydroxybenzamide (1 g, 3.31 mmol, 22.86% yield, 82% purity) as a white solid. MS(M+H)⁺=248.2.

### Step 2. Synthesis of 4-((2-chloro-5-fluoropyrimidin-4-yl)oxy)-N-(2-chlorophenyl)benzamide (5)

To a solution of N-(2-chlorophenyl)-4-hydroxybenzamide (1 g, 3.31 mmol, 82% purity) in DMF (10 mL) were added K₂CO₃ (915.16 mg, 6.62 mmol) and 2,4-dichloro-5-fluoropyrimidine (552.79 mg, 3.31 mmol). The mixture was stirred at 25° C. for 2 hr. LCMS showed one main peak with desired mass. The reaction mixture was mixed with water (50 mL) and filtered. The filter cake was collected and triturated with MTBE/EtOAc (1:1) at 25° C. for 30 min to afford 4-((2-chloro-5-fluoropyrimidin-4-yl)oxy)-N-(2-chlorophenyl)benzamide (660 mg, 1.75 mmol, 52.71% yield, 98% purity) as a white solid and 810 mg crude product. The crude product was triturated with petroleum ether/EtOAc (3:1) at 25° C. for 30 min to afford 4-((2-chloro-5-fluoropyrimidin-4-yl)oxy)-N-(2-chlorophenyl)benzamide (570 mg, 1.15 mmol, 34.60% yield, 76% purity) as a white solid. MS(M+H)⁺=378.0.

### Step 3. Synthesis of methyl 4-((4-(4-((2-chlorophenyl)carbamoyl)phenoxy)-5-fluoropyrimidin-2-yl)amino)benzoate (7)

To a solution of 4-((2-chloro-5-fluoropyrimidin-4-yl)oxy)-N-(2-chlorophenyl)benzamide (570 mg, 1.15 mmol, 76% purity) and methyl 4-aminobenzoate (173.15 mg, 1.15 mmol) in dioxane (6 mL) were added Pd₂(dba)₃ (104.89 mg, 114.55 μmol), Cs₂CO₃ (1.12 g, 3.44 mmol) and Xantphos (66.28 mg, 114.55 μmol) under N₂. The mixture was stirred at 100° C. for 16 hr under N₂ atmosphere. LCMS showed one main peak with desired mass. The reaction mixture was diluted with water (30 mL) and extracted with EtOAc (30 mL×3). The combined organic layers were washed with water (30 mL), dried over Na₂SO₄, filtered and concentrated under reduced pressure. The residue was purified by flash silica gel chromatography (ISCO®; 20 g SepaFlash® Silica Flash Column, Eluent of 20-30% EtOAc/Petroleum ether gradient @ 80 mL/min) to afford methyl 4-((4-(4-((2-chlorophenyl)carbamoyl)phenoxy)-5-fluoropyrimidin-2-yl)amino)benzoate (520 mg, 896.76 μmol, 78.29% yield, 85% purity) as a yellow solid. MS(M+H)⁺=493.2.

Step 4. Synthesis of 4-((4-(4-((2-chlorophenyl)carbamoyl)phenoxy)-5-fluoropyrimidin-2-yl)amino) benzoic acid (8)

To a solution of methyl 4-((4-(4-((2-chlorophenyl)carbamoyl)phenoxy)-5-fluoropyrimidin-2-yl)amino)benzoate (200 mg, 344.91 μmol, 85% purity) in THF (5 mL) and H₂O (1 mL) was added LiOH·H₂O (43.42 mg, 1.03 mmol). The mixture was stirred at 40° C. for 40 hr. LCMS showed ~13% of methyl 4-((4-(4-((2-chlorophenyl)carbamoyl)phenoxy)-5-fluoropyrimidin-2-yl)amino)benzoate remained and ~53% of desired compound was detected. The reaction mixture was diluted with water (20 mL) and extracted with EtOAc (30 mL×3). The aqueous solution was adjusted to pH=5 by HCl (1 M). After extraction with EtOAc (30 mL×3). The combined organic layers dried over Na₂SO₄, filtered and concentrated under reduced pressure. The crude product was triturated with MTBE/EtOAc (10 mL, 1:1) at 25° C. for 10 min to afford 4-((4-(4-((2-chlorophenyl)carbamoyl)phenoxy)-5-fluoropyrimidin-2-yl)amino)benzoic acid (120 mg, 150.36 μmol, 43.59% yield, 60% purity) as a white solid. MS(M+H)⁺=479.2.

### Step 5. Synthesis of N-(2-chlorophenyl)-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)oxy)benzamide (Compound 101)

To a solution of 4-((4-(4-((2-chlorophenyl)carbamoyl)phenoxy)-5-fluoropyrimidin-2-yl)amino) benzoic acid (120 mg, 150.36 μmol, 60% purity) in DMF (2 mL) were added HATU (85.76 mg, 225.54 μmol), DIPEA (97.16 mg, 751.79 μmol, 130.94 μL) and 3-(4-(4-(2-(1-aminopiperidin-4-yl)ethyl)piperazin-1-yl)phenyl)piperidine-2,6-dione (98.33 mg, 225.54 μmol, HCl salt). The mixture was stirred at 25° C. for 1 hr. LCMS showed one main peak with desired mass. The reaction mixture was poured into water (10 mL) and filtered. The filter cake was collected and purified by prep-HPLC (column: Phenomenex luna C18 150*25 mm*10 m; mobile phase: [water(TFA)-ACN]; gradient: 31%-61% B over 10 min), the eluent was lyophilized to afford N-(2-chlorophenyl)-4-((2-((4-((4-(2-(4-(4-(2,6-dioxopiperidin-3-yl)phenyl)piperazin-1-yl)ethyl)piperidin-1-yl)carbamoyl)phenyl)amino)-5-fluoropyrimidin-4-yl)oxy)benzamide (36.7 mg, 28.14 μmol, 18.72% yield, 92.2% purity, 3TFA salt) as a white solid. MS(M+H)⁺=860.4.

¹H NMR (400 MHz, DMSO-d₆) δ=10.79 (s, 1H), 10.19 (s, 1H), 9.89 (s, 1H), 9.53-9.40 (m, 1H), 9.29-9.16 (m, 1H), 8.59 (d, J=2.6 Hz, 1H), 8.16 (br d, J=8.4 Hz, 2H), 7.65 (br d, J=7.9 Hz, 1H), 7.58 (br d, J=7.6 Hz, 1H), 7.56-7.52 (m, 2H), 7.52-7.46 (m, 3H), 7.42 (br t, J=7.4 Hz, 1H), 7.37-7.30 (m, 1H), 7.12 (br d, J=8.4 Hz, 2H), 6.98 (br d, J=8.6 Hz, 2H), 3.88-3.80 (m, 2H), 3.76 (br dd, J=5.0, 11.4 Hz, 1H), 3.62-3.58 (m, 2H), 3.24-3.08 (m, 5H), 3.04-2.92 (m, 4H), 2.70-2.61 (m, 3H), 2.21-2.09 (m, 1H), 2.04-1.95 (m, 1H), 1.75-1.56 (m, 4H), 1.38-1.23 (m, 3H).

### Example 102. Synthesis of 4-[[4-[4-[(2-chlorophenyl)-methyl-carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]benzamide (Compound 102)

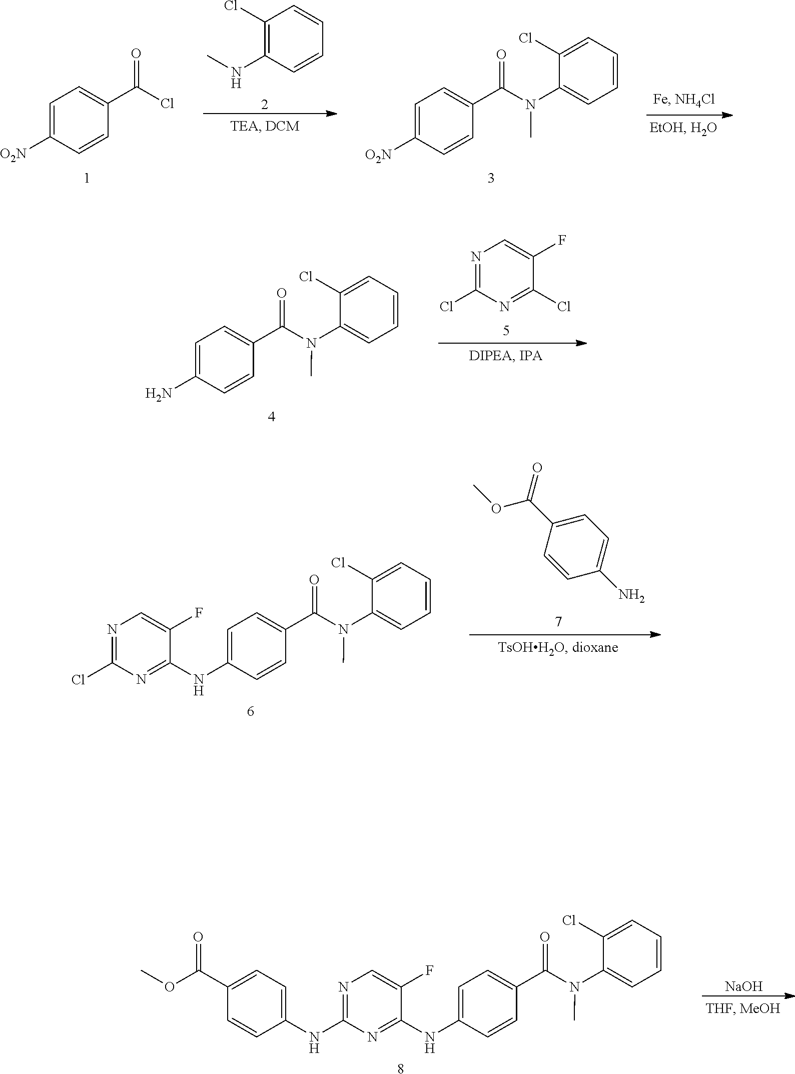

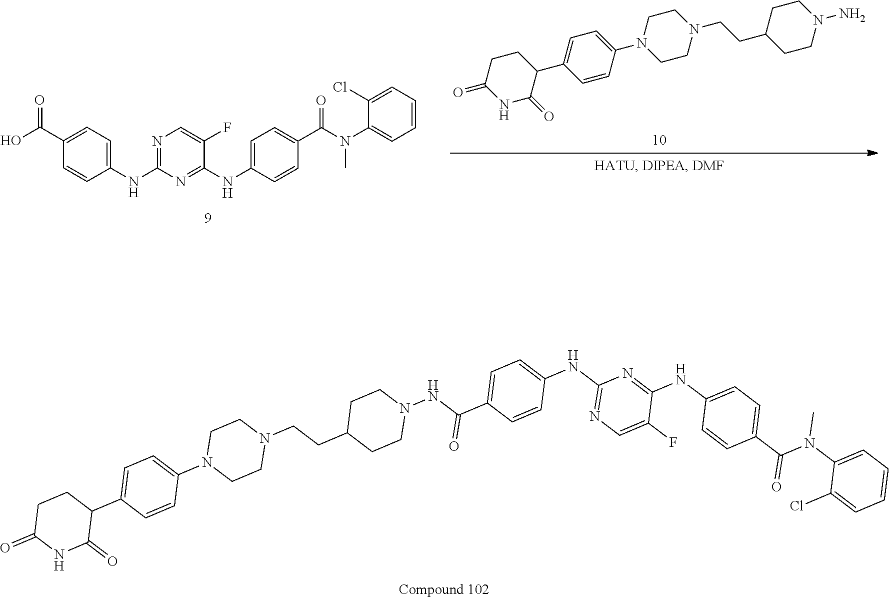 [SMILES: CN(C(=O)c1ccc(Nc2nc(Nc3ccc(C(=O)O)cc3)ncc2F)cc1)c1ccccc1Cl ; NN1CCC(CCN2CCN(c3ccc(C4CCC(=O)NC4=O)cc3)CC2)CC1 ; CN(C(=O)c1ccc(Nc2nc(Nc3ccc(C(=O)NN4CCC(CCN5CCN(c6ccc(C7CCC(=O)NC7=O)cc6)CC5)CC4)cc3)ncc2F)cc1)c1ccccc1Cl]

### Step 1. Synthesis of N-(2-chlorophenyl)-N-methyl-4-nitro-benzamide (3)

To a solution of 4-nitrobenzoyl chloride (2.04 g, 11.02 mmol) and TEA (1.86 g, 18.36 mmol, 2.56 mL) in DCM (20 mL) was added 2-chloro-N-methyl-aniline (1.3 g, 9.18 mmol) in DCM (20 mL) slowly under N₂ atmosphere at 0° C. The mixture was stirred at 20° C. for 16 hr under N₂ atmosphere. LCMS showed a peak (29%) with the mass of 2-chloro-N-methyl-aniline and a peak (23%) with desired mass. To the mixture was added TEA (1.11 g, 11.02 mmol, 1.53 mL) and 4-nitrobenzoyl chloride (1.7 g, 9.18 mmol) in DCM (10 mL). The mixture was stirred at 20° C. for 3 hr. LCMS showed a peak (29%) with the mass of 2-chloro-N-methyl-aniline and a peak (21%) with desired mass. The reaction mixture was diluted with 40 mL of water and extracted with DCM (60 mL×2). The combined organic phase was concentrated under reduced pressure. The residue was purified by flash silica gel chromatography (12 g SepaFlash® Silica Flash Column, Eluent of 0-33% EtOAc/Petroleum ether, gradient @ 60 mL/min) to afford N-(2-chlorophenyl)-N-methyl-4-nitro-benzamide (900 mg, 2.88 mmol, 31.36% yield, 93% purity) as light yellow solid. MS(M+H)⁺=291.0.

### Step 2. Synthesis of 4-amino-N-(2-chlorophenyl)-N-methyl-benzamide (4)

To a solution of N-(2-chlorophenyl)-N-methyl-4-nitro-benzamide (900 mg, 3.10 mmol) and NH₄Cl (828.03 mg, 15.48 mmol) in EtOH (10 mL) and H₂O (10 mL) was added Fe (518.68 mg, 9.29 mmol). The mixture was stirred at 80° C. for 2 hr. LCMS showed a peak (95%) with desired mass. The reaction mixture was filtrated through celite and concentrated under reduced pressure to remove organic phase. The residue was diluted with 30 mL of aqueous NaHCO₃ and extracted with EtOAc (50 mL×3). The combined organic layer was dried over Na₂SO₄ and concentrated under reduced pressure to afford 4-amino-N-(2-chlorophenyl)-N-methyl-benzamide (800 mg, crude) as yellow oil. MS(M+H)⁺=261.0.

### Step 3. Synthesis of 4-[(2-chloro-5-fluoro-pyrimidin-4-yl)amino]-N-(2-chlorophenyl)-N-methyl-benzamide

To a solution of 4-amino-N-(2-chlorophenyl)-N-methyl-benzamide (800 mg, 3.07 mmol) in IPA (8 mL) was added DIPEA (1.19 g, 9.21 mmol, 1.60 mL) and 2,4-dichloro-5-fluoro-pyrimidine (512.33 mg, 3.07 mmol). The mixture was stirred at 80° C. for 16 hr. LCMS showed a peak (28%) with the mass of 4-amino-N-(2-chlorophenyl)-N-methyl-benzamide and a peak (52%) with desired mass. The mixture was stirred at 80° C. for 24 hr. LCMS showed a peak (73%) with desired mass. The reaction mixture was filtered and washed with 2 mL of MeCN. The filter cake was collected and dried to afford 4-[(2-chloro-5-fluoro-pyrimidin-4-yl)amino]-N-(2-chlorophenyl)-N-methyl-benzamide (900 mg, 2.28 mmol, 74.22% yield, 99% purity) as white solid. MS(M+H)⁺=391.0.

¹H NMR (400 MHz, CDCl₃-d) δ=8.06 (d, J=2.5 Hz, 1H), 7.49 (br d, J=8.1 Hz, 2H), 7.43-7.31 (m, 3H), 7.25-7.05 (m, 4H), 3.39 (s, 3H).

### Step 4. Synthesis of methyl 4-[[4-[4-[(2-chlorophenyl)carbamoyl]-N-methyl-anilino]-5-fluoro-pyrimidin-2-yl]amino]benzoate (8)

To a solution of 4-[(2-chloro-5-fluoro-pyrimidin-4-yl) amino]-N-(2-chlorophenyl)-N-methyl-benzamide (500 mg, 1.28 mmol) in dioxane (10 mL) was added TsOH·H₂O (729.31 mg, 3.83 mmol) and methyl 4-aminobenzoate (193.19 mg, 1.28 mmol). The mixture was stirred at 100° C. for 16 hr. LCMS showed starting material was consumed and a peak (57%) with desired mass. The reaction mixture was diluted with 30 mL of aqueous NaHCO₃ solution and extracted with EtOAc (30 mL×3). The combined organic phase was concentrated under reduced pressure to afford methyl 4-[[4-[4-[(2-chlorophenyl)carbamoyl]-N-methyl-anilino]-5-fluoro-pyrimidin-2-yl]amino]benzoate (600 mg, 806.44 μmol, 63.10% yield, 68% purity) as yellow solid. MS(M+H)⁺=506.1.

### Step 5. Synthesis of 4-[[4-[4-[(2-chlorophenyl)carbamoyl]-N-methyl-anilino]-5-fluoro-pyrimidin-2-yl]amino]benzoic acid (9)

To a solution of methyl 4-[[4-[4-[(2-chlorophenyl)carbamoyl]-N-methyl-anilino]-5-fluoro-pyrimidin-2-yl]amino]benzoate (600 mg, 1.19 mmol) in MeOH (4 mL) and THF (16 mL) was added NaOH (3 M, 4 mL). The mixture was stirred at 40° C. for 16 hr. LCMS showed a peak (54%) with desired mass. To the reaction mixture was added 1 M HCl to adjust pH=7 and concentrated under reduced pressure to remove organic phase. The residue was filtered and washed with water (10 mL). The filter cake was collected and dried to afford 4-[[4-[4-[(2-chlorophenyl) carbamoyl]-N-methyl-anilino]-5-fluoro-pyrimidin-2-yl]amino]benzoic acid (560 mg, 1.09 mmol, 92.15% yield, 96% purity) as yellow solid. MS(M+H)⁺=492.1.

### Step 6. Synthesis of 4-[[4-[4-[(2-chlorophenyl)-methyl-carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]benzamide (Compound 102)

To a solution of 3-[4-[4-[2-(1-amino-4-piperidyl)ethyl]piperazin-1-yl]phenyl]piperidine-2, 6-dione (106.36 mg, 243.95 μmol, HCl) and 4-[[4-[4-[(2-chlorophenyl)carbamoyl]-N-methyl-anilino]-5-fluoro-pyrimidin-2-yl]amino]benzoic acid (100 mg, 203.29 μmol) in DMF (2 mL) were added HATU (115.95 mg, 304.94 μmol) and DIPEA (78.82 mg, 609.88 μmol, 106.23 μL). The mixture was stirred at 20° C. for 2 hr. LCMS showed a peak (22%) with desired mass. The reaction mixture was diluted with 10 mL of water and filtrated. The filtrate cake was purified by prep-HPLC (column: Phenomenex luna C18 150*25 mm*10 um; mobile phase: [water (TFA)-ACN]; gradient: 21%-51% B over 10 min) and lyophilized to afford 4-[[4-[4-[(2-chlorophenyl)-methyl-carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]benzamide (19.2 mg, 14.37 μmol, 7.07% yield, 91% purity, 3TFA) as white solid. MS(M+H)⁺=873.4.

¹H NMR (400 MHz, DMSO-d₆) δ=10.79 (s, 1H), 9.72-9.39 (m, 4H), 8.18 (br d, J=2.9 Hz, 1H), 7.80-7.62 (m, 6H), 7.48 (br t, J=6.6 Hz, 2H), 7.36-7.21 (m, 4H), 7.12 (br d, J=8.4 Hz, 2H), 6.98 (br d, J=8.1 Hz, 2H), 3.89-3.74 (m, 4H), 3.27 (s, 3H), 3.22-3.13 (m, 5H), 3.01-2.93 (m, 2H), 2.91-2.77 (m, 2H), 2.71-2.58 (m, 2H), 2.23-1.94 (m, 3H), 1.80-1.72 (m, 2H), 1.70-1.59 (m, 2H), 1.50-1.32 (m, 3H).

### Example 103. Synthesis of 4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-phenyl-benzamide (Compound 103)

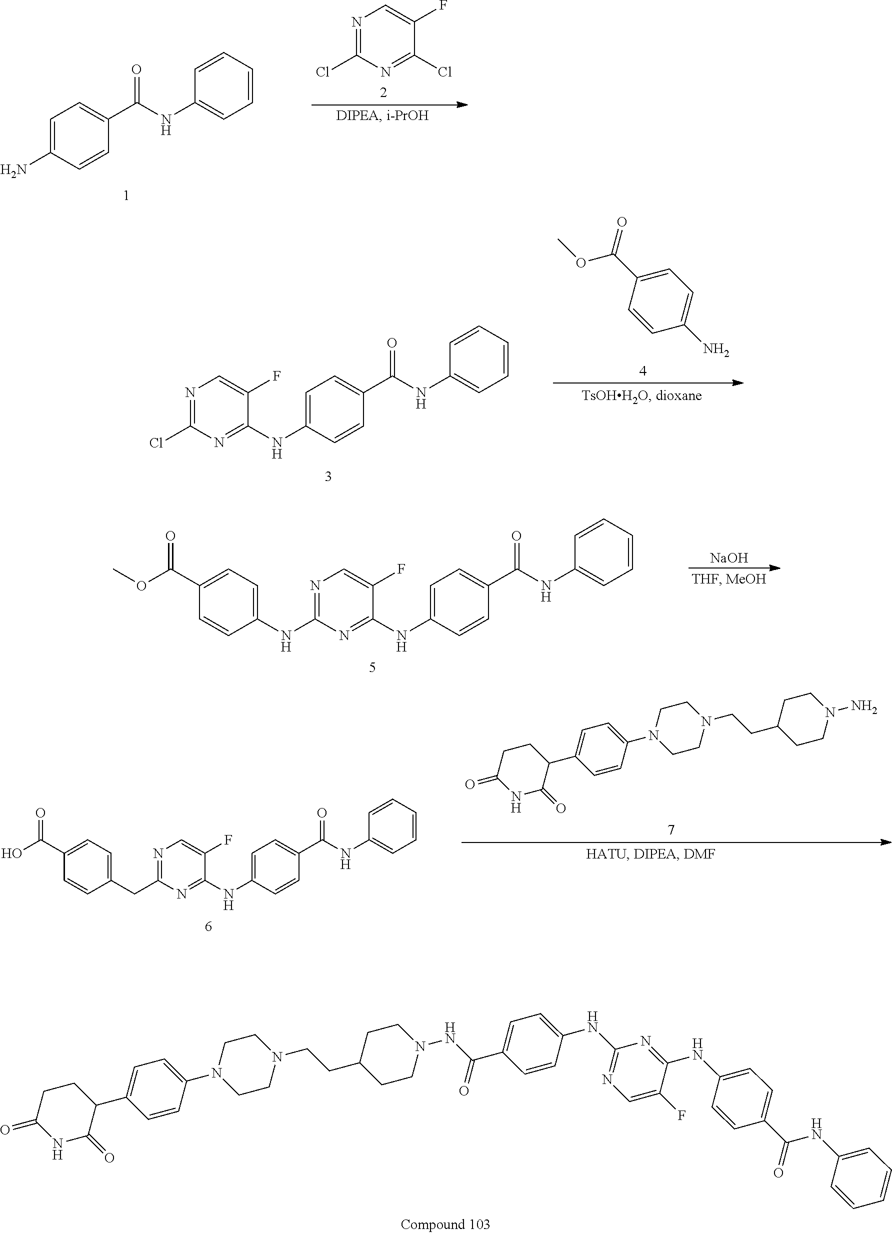

### Step 1. Synthesis of 4-[(2-chloro-5-fluoro-pyrimidin-4-yl)amino]-N-phenyl-benzamide (3)

To a solution of 4-amino-N-phenyl-benzamide (500 mg, 2.36 mmol) in propan-2-ol (5 mL) was added DIPEA (913.39 mg, 7.07 mmol, 1.23 mL) and 2,4-dichloro-5-fluoro-pyrimidine (393.34 mg, 2.36 mmol). The mixture was stirred at 80° C. for 16 hr. LCMS showed a peak (46%) with starting material and a peak (39%) with desire mass. The mixture was stirred at 80° C. for 56 hr. LCMS showed a peak (63%) with desired mass and most of starting material was consumed. The reaction mixture was concentrated under reduced pressure to afford a residue. The residue was purified by flash silica gel chromatography (12 g SepaFlash® Silica Flash Column, Eluent of 0-33% EtOAc/Petroleum ether, gradient @ 60 mL/min) to afford the crude product, which was triturated with 5 mL of EtOAc and 20 mL of petroleum ether to afford 4-[(2-chloro-5-fluoro-pyrimidin-4-yl)amino]-N-phenyl-benzamide (700 mg, 1.76 mmol, 74.56% yield, 86% purity) as white solid. MS(M+H)⁺=343.0.

¹H NMR (400 MHz, DMSO-d₆) δ=10.27 (s, 1H), 10.18 (s, 1H), 8.41 (d, J=3.4 Hz, 1H), 7.99 (d, J=8.8 Hz, 2H), 7.87 (d, J=8.8 Hz, 2H), 7.78 (d, J=7.6 Hz, 2H), 7.36 (t, J=7.9 Hz, 2H), 7.10 (t, J=7.4 Hz, 1H).

### Step 2. Synthesis of methyl 4-[[5-fluoro-4-[4-(phenylcarbamoyl)anilino]pyrimidin-2-yl]amino]benzoate (5)

To a solution of 4-[(2-chloro-5-fluoro-pyrimidin-4-yl) amino]-N-phenyl-benzamide (650 mg, 1.90 mmol) in dioxane (10 mL) was added TsOH·H₂O (1.08 g, 5.69 mmol) and methyl 4-aminobenzoate (286.66 mg, 1.90 mmol). The mixture was stirred at 100° C. for 16 hr. LCMS showed a peak (55%) with desired mass. To the mixture was added 20 mL of water and the mixture was filtered. The filter cake was collected and dried to afford methyl 4-[[5-fluoro-4-[4-(phenylcarbamoyl)anilino]pyrimidin-2-yl]amino]benzoate (360 mg, 755.48 μmol, 39.84% yield, 96% purity) as light yellow solid. MS(M+H)⁺=458.1.

### Step 3. Synthesis of 4-[[5-fluoro-4-[4-(phenylcarbamoyl)anilino]pyrimidin-2-yl]amino]benzoic acid (6)

To a solution of methyl 4-[[5-fluoro-4-[4-(phenylcarbamoyl)anilino]pyrimidin-2-yl]amino]benzoate (400 mg, 874.40 μmol) in THF (12 mL) and MeOH (3 mL) was added NaOH (3 M, 3 mL). The mixture was stirred at 40° C. for 40 hr. LCMS showed a peak (56%) with desired mass. The mixture was concentrated to remove the organic solvent. The residue was neutralized to pH=7 by 2N HCl solution. The resulting mixture was filtered and washed with water (10 mL). The filter cake was collected and dried to afford 4-[[5-fluoro-4-[4-(phenylcarbamoyl)anilino]pyrimidin-2-yl]amino]benzoic acid (250 mg, 428.48 μmol, 49.00% yield, 76% purity) as white solid. MS(M+H)⁺=444.1.

### Step 4. Synthesis of 4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-phenyl-benzamide (Compound 103)

To a solution of 4-[[5-fluoro-4-[4-(phenylcarbamoyl)anilino]pyrimidin-2-yl]amino]benzoic acid (60 mg, 135.31 μmol) and 3-[4-[4-[2-(1-amino-4-piperidyl)ethyl]piperazin-1-yl]phenyl]piperidine-2, 6-dione (58.99 mg, 135.31 μmol, HCl) in DMF (2 mL) were added HATU (61.74 mg, 162.37 mol) and DIPEA (52.46 mg, 405.93 μmol, 70.71 μL). The mixture was stirred at 20° C. for 16 hr. LCMS showed a peak (24%) with desired mass. The reaction mixture was diluted with 20 mL of water and filtered. The filter cake was collected and purified by prep-HPLC (column: Phenomenex luna C18 150*25 mm*10 um; mobile phase: [water (TFA)-ACN]; gradient: 19%-49% B over 10 min). The eluent was lyophilized to afford 4-[[2-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]-N-phenyl-benzamide (12.2 mg, 10.04 μmol, 7.42% yield, 96% purity, 3TFA) as white solid. MS(M+H)⁺=825.4.

¹H NMR (400 MHz, DMSO-d₆) δ=10.79 (s, 1H), 10.15 (s, 1H), 9.82-9.63 (m, 2H), 9.62-9.39 (m, 1H), 8.25 (d, J=3.5 Hz, 1H), 8.07-7.93 (m, 4H), 7.85-7.65 (m, 6H), 7.36 (t, J=7.9 Hz, 2H), 7.18-7.05 (m, 3H), 6.98 (br d, J=8.8 Hz, 2H), 3.86-3.75 (m, 5H), 3.23-3.07 (m, 6H), 3.02-2.80 (m, 4H), 2.75-2.57 (m, 2H), 2.22-2.09 (m, 1H), 2.04-1.93 (m, 1H), 1.83-1.58 (m, 4H), 1.42-1.32 (m, 3H).

### Example 104. Synthesis of N-[4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]phenyl]-2-fluoro-benzamide (Compound 104)

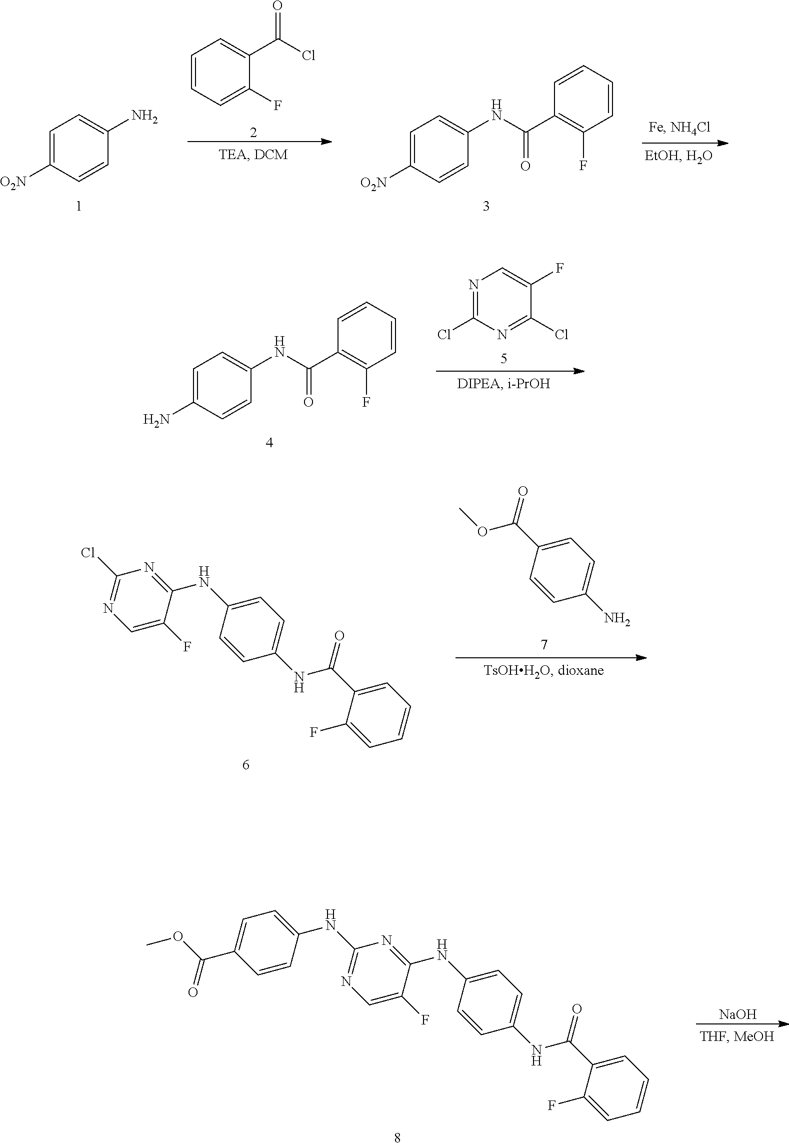

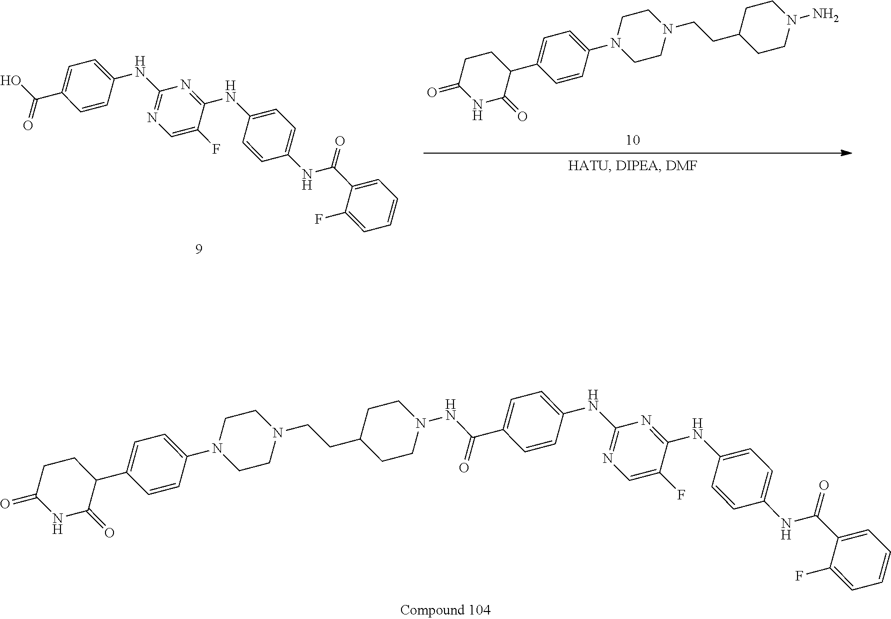 [SMILES: O=C(O)c1ccc(Nc2ncc(F)c(Nc3ccc(NC(=O)c4ccccc4F)cc3)n2)cc1 ; NN1CCC(CCN2CCN(c3ccc(C4CCC(=O)NC4=O)cc3)CC2)CC1 ; "9" ; O=C1CCC(c2ccc(N3CCN(CCC4CCN(NC(=O)c5ccc(Nc6ncc(F)c(Nc7ccc(NC(=O)c8ccccc8F)cc7)n6)cc5)CC4)CC3)cc2)C(=O)N1]

### Step 1. Synthesis of 2-fluoro-N-(4-nitrophenyl)benzamide (3)

To a solution of 4-nitroaniline (4 g, 28.96 mmol, 2.78 mL) and TEA (5.86 g, 57.92 mmol, 8.06 mL) in DCM (30 mL) was added 2-fluorobenzoyl chloride (5.51 g, 34.75 mmol, 4.12 mL) in DCM (30 mL) slowly at 0′° C. The mixture was stirred at 20° C. for 16 hr. LCMS showed a peak (41%) with desired mass. The reaction was diluted with 50 mL of water and extracted with 60 mL of EtOAc. The combined organic phase was concentrated under reduced pressure. The residue was triturated with EtOAc/petroleum ether (50 mL, 1/5) to afford 2-fluoro-N-(4-nitrophenyl)benzamide (5 g, 14.41 mmol, 49.76% yield, 75% purity) as yellow solid. MS(M+H)⁺=261.0.

### Step 2. Synthesis of N-(4-aminophenyl)-2-fluoro-benzamide (4)

To a solution of 2-fluoro-N-(4-nitrophenyl) benzamide (1 g, 3.84 mmol) in EtOH (10 mL) and H₂O (10 mL) were added Fe (643.82 mg, 11.53 mmol) and NH₄Cl (1.03 g, 19.21 mmol). The mixture was stirred at 80° C. for 1 hr. LCMS showed several peaks (67%) with desired mass. The reaction mixture was filtered through celite and concentrated under reduced pressure. The residue was diluted with 40 mL of aqueous NaHCO₃ solution and extracted with EtOAc (40 mL×2) and DCM (40 mL). The combined organic phase was concentrated under reduced pressure to afford N-(4-aminophenyl)-2-fluoro-benzamide (800 mg, 3.13 mmol, 81.38% yield, 90% purity) as yellow solid. MS(M+H)⁺=231.1.

¹H NMR (400 MHz, DMSO-d₆) δ=9.95 (s, 1H), 7.67-7.47 (m, 2H), 7.41-7.19 (m, 4H), 6.54 (br d, J=8.6 Hz, 2H), 4.93 (s, 2H)

### Step 3. Synthesis of N-[4-[(2-chloro-5-fluoro-pyrimidin-4-yl)amino]phenyl]-2-fluoro-benzamide (6)

To a solution of N-(4-aminophenyl)-2-fluoro-benzamide (800 mg, 3.47 mmol) in propan-2-ol (8 mL) was added DIPEA (1.35 g, 10.42 mmol, 1.82 mL) and 2,4-dichloro-5-fluoro-pyrimidine (580.16 mg, 3.47 mmol). The mixture was stirred at 80° C. for 16 hr. LCMS showed a peak (93%) with desired mass. The reaction mixture was filtered and washed with 3 mL of MeCN. The filter cake was collected and dried to afford N-[4-[(2-chloro-5-fluoro-pyrimidin-4-yl)amino]phenyl]-2-fluoro-benzamide (1 g, 2.72 mmol, 78.18% yield, 98% purity) as light yellow solid. MS(M+H)⁺=361.0.

¹H NMR (400 MHz, DMSO-d₆) δ=10.44 (s, 1H), 10.00 (s, 1H), 8.30 (d, J=3.3 Hz, 1H), 7.80-7.51 (m, 6H), 7.43-7.26 (m, 2H).

### Step 4. Synthesis of methyl 4-[[5-fluoro-4-[4-[(2-fluorobenzoyl)amino]anilino]pyrimidin-2-yl]amino]benzoate (8)

To a solution of N-[4-[(2-chloro-5-fluoro-pyrimidin-4-yl)amino]phenyl]-2-fluoro-benzamide (500 mg, 1.39 mmol) in dioxane (5 mL) was added TsOH·H₂O (790.93 mg, 4.16 mmol) and methyl 4-aminobenzoate (209.51 mg, 1.39 mmol). The mixture was stirred at 100° C. for 16 hr. LCMS showed a peak (49%) with desired mass. The reaction mixture was diluted with 30 mL of water and filtered. The filter cake was collected and dried to afford methyl 4-[[5-fluoro-4-[4-[(2-fluorobenzoyl) amino]anilino]pyrimidin-2-yl]amino]benzoate (600 mg, 795.04 μmol, 57.36% yield, 63% purity) as yellow solid. MS(M+H)⁺=476.1.

### Step 5. Synthesis of 4-[[5-fluoro-4-[4-[(2-fluorobenzyl)amino]anilino]pyrimidin-2-yl]amino] benzoic acid (9)

To a solution of methyl 4-[[5-fluoro-4-[4-[(2-fluorobenzoyl)amino]anilino]pyrimidin-2-yl]amino]benzoate (300 mg, 630.99 μmol) in MeOH (2 mL) and THF (8 mL) was added NaOH (3 M, 2 mL). The mixture was stirred at 40° C. for 16 hr. LCMS showed three peaks (96%) with desired mass. To the reaction mixture was added 1 M HCl to adjust pH=7 and filtered. The filter cake was collected and dried to afford 4-[[5-fluoro-4-[4-[(2-fluorobenzoyl) amino]anilino]pyrimidin-2-yl]amino]benzoic acid (150 mg, 243.81 μmol, 38.64% yield, 75% purity) as light yellow solid. MS(M+H)⁺=462.1.

### Step 6. Synthesis of N-[4-[[2-[4-[[4-[2-[4-[4-(2,6-dioxo-3-piperidyl)phenyl]piperazin-1-yl]ethyl]-1-piperidyl] carbamoyl]anilino]-5-fluoro-pyrimidin-4-yl]amino]phenyl]-2-fluoro-benzamide (Compound 104)

To a solution of 4-[[5-fluoro-4-[4-[(2-fluorobenzoyl)amino]anilino]pyrimidin-2-yl]amino]benzoic acid (150 mg, 325.08 μmol) and 3-[4-[4-[2-(1-amino-4-piperidyl)ethyl]piperazin-1-yl]phenyl]piperidine-2,6-dione (127.56 mg, 292.58 μmol, HCl) in DMF (2 mL) was added HATU (148.33 mg, 390.10 mol) and DIPEA (126.04 mg, 975.25 μmol, 169.87 μL). The mixture was stirred at 20° C. for 16 hr. LCMS showed a peak (40%) with desired mass. The reaction mixture was diluted with 30 mL of water and filtered. The filter cake was collected and purified by prep-HPLC (column: Phenomenex luna C18 150*25 mm*10 um; mobile phase: [water (TFA)-ACN]; gradient: 19%-49% B over 10 min) and lyophilized to afford N-[4-[[2-[4-[[4-[2-[4-[4-(2,6-dioxo-3-piperidyl)phenyl]piperazin-1-yl]ethyl]-1-piperidyl]carbamoyl]anilino]-5-fluoro-pyrimidin-4-yl]amino]phenyl]-2-fluoro-benzamide (57.4 mg, 47.47 μmol, 14.60% yield, 98% purity, 3TFA) as yellow solid. MS(M+H)⁺=843.3.

¹H NMR (400 MHz, DMSO-d₆) δ=10.80 (s, 1H), 10.44 (s, 1H), 9.89 (br s, 1H), 9.74-9.46 (m, 3H), 8.17 (d, J=3.7 Hz, 1H), 7.87-7.66 (m, 8H), 7.63-7.54 (m, 1H), 7.45-7.27 (m, 2H), 7.13 (br d, J=8.6 Hz, 2H), 6.99 (br d, J=8.6 Hz, 2H), 3.63-3.60 (m, 5H), 3.29-3.08 (m, 6H), 3.05-2.81 (m, 4H), 2.74-2.58 (m, 2H), 2.23-2.09 (m, 1H), 2.07-1.95 (m, 1H), 1.87-1.58 (m, 4H), 1.45-1.38 (m, 3H).

### Example 105. Synthesis of 4-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]-2-fluoro-benzamide (Compound 105)

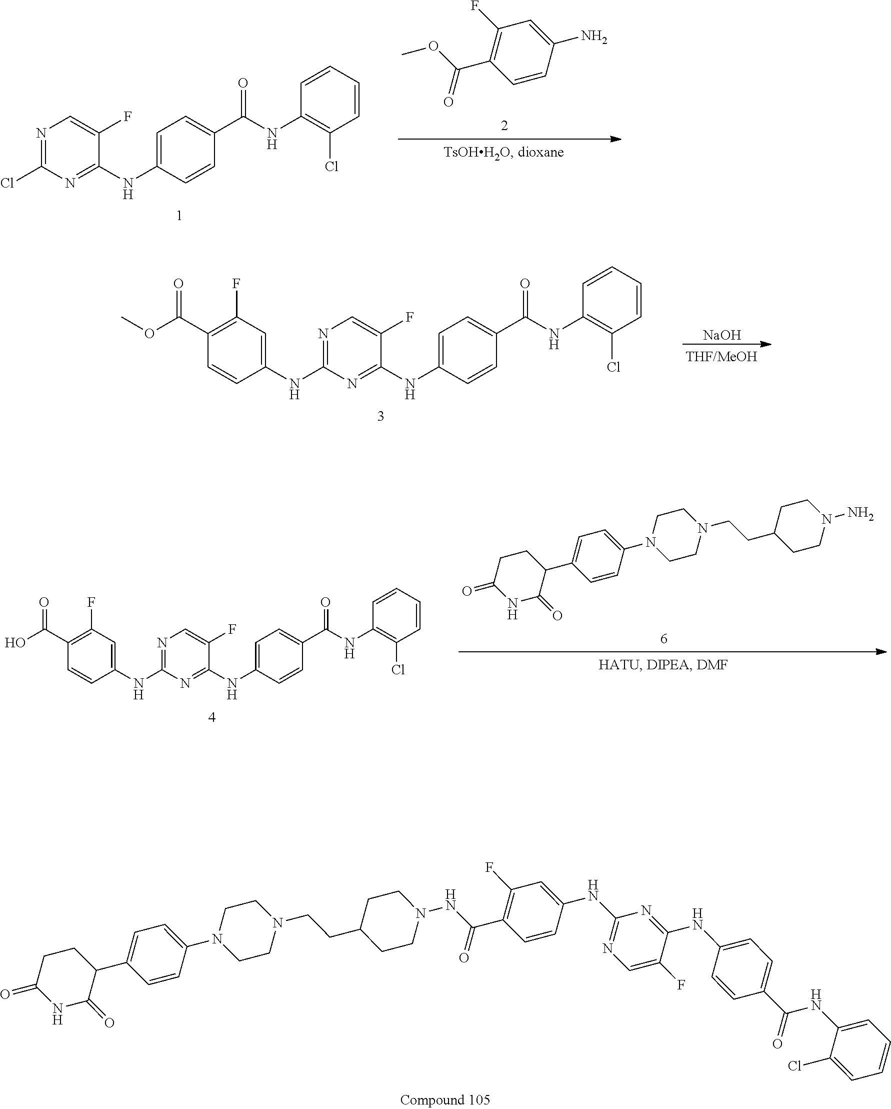

### Step 1. Synthesis of methyl 4-[[4-[4-[(2-chlorophenyl)carbamoyl]anilino]-5-fluoro-pyrimidin-2-yl]amino]-2-fluoro-benzoate (3)

To a solution of 4-[(2-chloro-5-fluoro-pyrimidin-4-yl) amino]-N-(2-chlorophenyl)benzamide (400 mg, 1.06 mmol) and methyl 4-amino-2-fluoro-benzoate (179.38 mg, 1.06 mmol) in dioxane (5 mL) was added TsOH·H₂O (605.15 mg, 3.18 mmol). The mixture was stirred at 100° C. for 16 hr. LCMS showed a peak (33%) with desired mass. The reaction mixture was diluted with 30 mL of water and filtered. The filter cake was triturated with 10 mL of MeCN to afford methyl 4-[[4-[4-[(2-chlorophenyl)carbamoyl]anilino]-5-fluoro-pyrimidin-2-yl]amino]-2-fluoro-benzoate (300 mg, 523.64 μmol, 49.38% yield, 89% purity) as yellow solid. MS(M+H)⁺=510.1.

### Step 2. Synthesis of 4-[[4-[4-[(2-chlorophenyl)carbamoyl]anilino]-5-fluoro-pyrimidin-2-yl]amino]-2-fluoro-benzoic acid (4)

To a solution of methyl 4-[[4-[4-[(2-chlorophenyl) carbamoyl]anilino]-5-fluoro-pyrimidin-2-yl]amino]-2-fluoro-benzoate (300 mg, 588.36 μmol) in THF (8 mL) and MeOH (2 mL) was added NaOH (3 M, 2 mL). The mixture was stirred at 40° C. for 19 hr. LCMS showed a peak (87%) with desired mass. To the reaction mixture was added 1 M HCl to adjust pH=7 and concentrated under reduced pressure to remove organic phase. The residue was filtered and washed with water (10 mL). The filter cake was collected and dried to afford 4-[[4-[4-[(2-chlorophenyl)carbamoyl]anilino]-5-fluoro-pyrimidin-2-yl]amino]-2-fluoro-benzoic acid (200 mg, 403.34 μmol, 68.55% yield) as white solid. MS(M+H)⁺=496.1.

### Step 3. Synthesis of 4-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]-2-fluoro-benzamide (Compound 105)

To a solution of 4-[[4-[4-[(2-chlorophenyl)carbamoyl]anilino]-5-fluoro-pyrimidin-2-yl]amino]-2-fluoro-benzoic acid (100 mg, 201.67 μmol) in DMF (2 mL) was added HATU (92.02 mg, 242.00 μmol), DIPEA (78.19 mg, 605.00 μmol, 105.38 μL) and 3-[4-[4-[2-(1-amino-4-piperidyl)ethyl]piperazin-1-yl]phenyl]piperidine-2,6-dione (87.93 mg, 201.67 μmol, HCl). The resulting mixture was stirred at 20° C. for 16 hr. LCMS showed a peak (56%) with desired mass. The reaction mixture was diluted with 10 mL of water and filtered. The filter cake was purified by prep-HPLC (column: Phenomenex luna C18 150*25 mm*10 um; mobile phase: [water (TFA)-ACN]; gradient: 27%-57% B over 10 min) and lyophilized to afford 4-[[4-[4-[(2-chlorophenyl)carbamoyl]anilino]-5-fluoro-pyrimidin-2-yl]amino]-N-[4-[2-[4-[4-(2,6-dioxo-3-piperidyl)phenyl]piperazin-1-yl]ethyl]-1-piperidyl]-2-fluoro-benzamide (18.4 mg, 16.65 μmol, 8.25% yield, 98% purity, 2TFA) as white solid. MS(M+H)⁺=877.3.

¹H NMR (400 MHz, DMSO-d₆) δ=10.79 (s, 1H), 9.92 (s, 1H), 9.81 (s, 2H), 9.66-9.43 (m, 1H), 9.10 (br s, 1H), 8.28 (d, J=3.5 Hz, 1H), 8.13-7.94 (m, 4H), 7.83 (br d, J=14.3 Hz, 1H), 7.65 (br d, J=6.6 Hz, 1H), 7.56 (dd, J=1.3, 8.0 Hz, 1H), 7.52-7.36 (m, 3H), 7.34-7.23 (m, 1H), 7.12 (br d, J=8.6 Hz, 2H), 7.03-6.89 (m, 2H), 3.88-3.70 (m, 4H), 3.25-2.86 (m, 9H), 2.71-2.59 (m, 3H), 2.22-2.07 (m, 1H), 2.06-1.94 (m, 1H), 1.78-1.54 (m, 4H), 1.35-1.28 (m, 3H).

### Example 106. Synthesis of N-(2-chlorophenyl)-4-((2-((4-((4-(2-(4-(1-(2,6-dioxopiperidin-3-yl)-3-methyl-2-oxo-2,3-dihydro-1H-benzo[d]imidazol-5-yl)piperazin-1-yl)ethyl)piperidin-1-yl)carbamoyl)phenyl)amino)-5-fluoropyrimidin-4-yl)amino)benzamide (Compound 106)

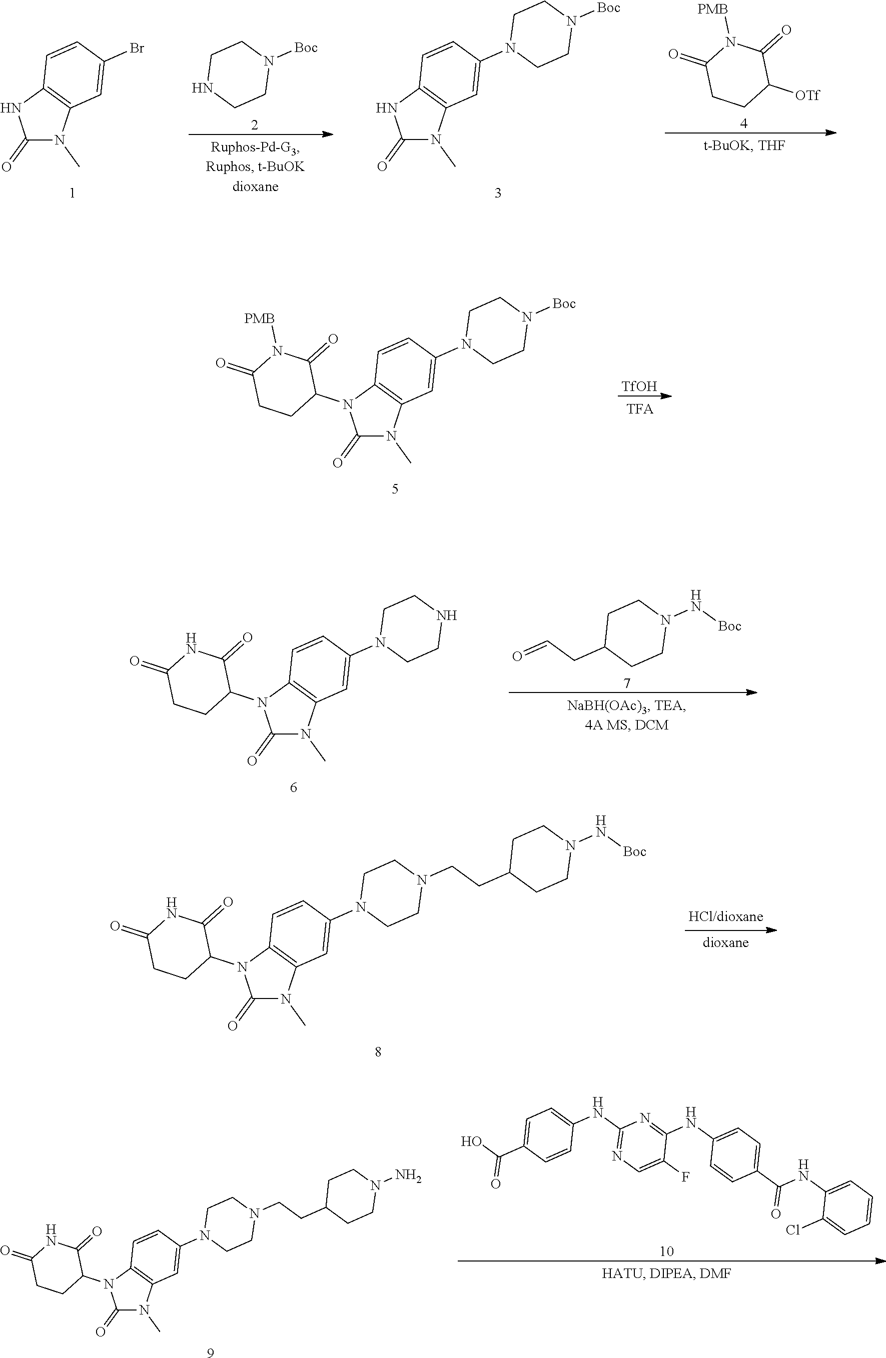

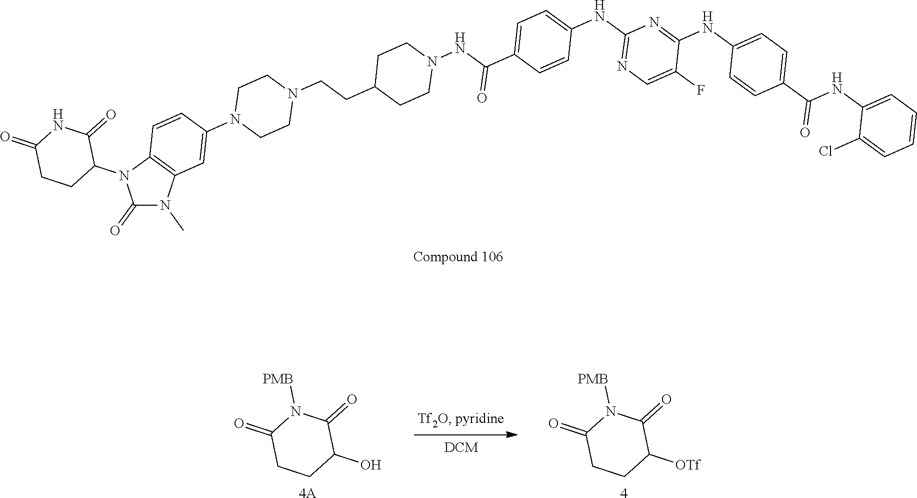

### Step 1. Synthesis of tert-butyl 4-(3-methyl-2-oxo-1H-benzimidazol-5-yl)piperazine-1-carboxylate (3)

To a mixture of 5-bromo-3-methyl-1H-benzimidazol-2-one (1 g, 4.40 mmol), tert-butyl piperazine-1-carboxylate (1.23 g, 6.61 mmol) and t-BuOK (1.24 g, 11.01 mmol) in dioxane (30 mL) were added RuPhos (102.76 mg, 220.21 μmol) and RuPhos-Pd-G₃ (184.17 mg, 220.21 μmol) at 25° C. under N₂ atmosphere. The resulting mixture was stirred at 90° C. for 2 hr. LCMS showed the starting material was consumed completely and a 79% peak with desired mass. The reaction mixture was poured into water (100 mL) and extracted with EtOAc (40 mL×3). The combined organic layers were washed with brine (50 mL), dried over Na₂SO₄ and concentrated. The crude product was purified by trituration with petroleum ether/EtOAc (20 mL, 10/1, stirred for 1 h). The mixture was filtered. The filter cake was collected and dried to afford tert-butyl 4-(3-methyl-2-oxo-1H-benzimidazol-5-yl)piperazine-1-carboxylate (1.2 g, 3.47 mmol, 78.69% yield, 96% purity) as yellow solid.

¹H NMR (400 MHz, DMSO-d₆) δ=10.55 (s, 1H), 6.83 (d, J=8.4 Hz, 1H), 6.77 (d, J=2.0 Hz, 1H), 6.60 (dd, J=2.2, 8.4 Hz, 1H), 3.47-3.44 (br d, J=5.0 Hz, 4H), 3.23 (s, 3H), 3.02-2.97 (m, 4H), 1.42 (s, 9H).

### Step 2. Synthesis of 1-(4-methoxybenzyl)-2,6-dioxopiperidin-3-yl trifluoromethanesulfonate (4)

To a solution of 3-hydroxy-1-[(4-methoxyphenyl)methyl]piperidine-2,6-dione (1 g, 4.01 mmol) and pyridine (634.67 mg, 8.02 mmol, 647.63 μL) in DCM (10 mL) was added Tf₂O (1.70 g, 6.02 mmol, 992.89 μL) dropwise at 0° C. The mixture was stirred at −10° C. for 1.5 hours under N₂ atmosphere. TLC (Petroleum ether:EtOAc=2:1) indicated the starting material was consumed completely, and one major new spot with lower polarity was detected. The reaction mixture was diluted with H₂O (10 mL) and extracted with EtOAc (10 mL×3). The combined organic layers were washed with brine (20 mL×2), dried over Na₂SO₄, filtered and concentrated under reduced pressure. The residue was purified by flash silica gel chromatography (Biotage; 20 g SepaFlash® Silica Flash Column, Eluent of ~20% EtOAc/Petroleum ether, gradient @ 100 mL/min) to afford 1-(4-methoxybenzyl)-2,6-dioxopiperidin-3-yl trifluoromethanesulfonate (1.55 g, 3.86 mmol, 96.25% yield, 95% purity) as a yellow oil.

¹H NMR (400 MHz, CDCl₃) δ=7.34 (d, J=8.6 Hz, 2H), 6.82 (d, J=8.6 Hz, 2H), 5.29 (dd, J=5.6, 11.5 Hz, 1H), 4.88 (s, 2H), 3.77 (s, 3H), 2.98 (td, J=4.5, 17.9 Hz, 1H), 2.73 (ddd, J=5.5, 12.4, 18.0 Hz, 1H), 2.47-2.24 (m, 2H).

### Step 3. Synthesis of tert-butyl 4-(1-(1-(4-methoxybenzyl)-2,6-dioxopiperidin-3-yl)-3-methyl-2-oxo-2,3-dihydro-1H-benzo[d]imidazol-5-yl)piperazine-1-carboxylate (5)

To a solution of tert-butyl 4-(3-methyl-2-oxo-2,3-dihydro-1H-benzo[d]imidazol-5-yl)piperazine-1-carboxylate (900 mg, 2.71 mmol) in THF (20 mL) was added t-BuOK (1 M, 3.20 mL). The reaction mixture was stirred at 0° C. for 0.5 hr. Followed by the addition of 1-(4-methoxybenzyl)-2,6-dioxopiperidin-3-yl trifluoromethanesulfonate (1.32 g, 3.47 mmol) in THF (5 mL) dropwise. The resulting reaction mixture was stirred at 25° C. for 0.5 hr under N₂ atmosphere. LCMS showed ~80% peak with desired mass. The reaction mixture was diluted with aq. NH₄Cl (50 mL). The resulting solution was extracted with EtOAc (50 mL×3), washed with brine (100 mL) dried over Na₂SO₄ and concentrated. The residue was purified by flash silica gel chromatography (ISCO®; 20 g SepaFlash® Silica Flash Column, Eluent of ~42% EtOAc/Petroleum ether, gradient @ 80 mL/min) to afford tert-butyl 4-(1-(1-(4-methoxybenzyl)-2,6-dioxopiperidin-3-yl)-3-methyl-2-oxo-2,3-dihydro-1H-benzo[d]imidazol-5-yl)piperazine-1-carboxylate (1.4 g, 1.99 mmol, 74.42% yield, 80% purity) as a brown solid. MS(M+H)⁺=564.3.

¹H NMR (400 MHz, CDCl₃) δ=7.35 (d, J=8.6 Hz, 2H), 6.81 (d, J=8.6 Hz, 2H), 6.62 (d, J=2.0 Hz, 1H), 6.55 (dd, J=2.0, 8.5 Hz, 1H), 6.40 (d, J=8.5 Hz, 1H), 5.18 (dd, J=5.4, 13.1 Hz, 1H), 4.94 (s, 2H), 3.78 (s, 3H), 3.64-3.59 (m, 4H), 3.39 (s, 3H), 3.07-3.04 (m, 4H), 3.02-2.93 (m, 1H), 2.89-2.76 (m, 1H), 2.64-2.48 (m, 1H), 2.21-2.10 (m, 1H), 1.48 (s, 9H).

### Step 4. Synthesis of 3-(3-methyl-2-oxo-5-(piperazin-1-yl)-2,3-dihydro-1H-benzo[d]imidazol-1-yl)piperidine-2,6-dione (6)

To a solution of 4-(1-(1-(4-methoxybenzyl)-2,6-dioxopiperidin-3-yl)-3-methyl-2-oxo-2,3-dihydro-1H-benzo[d]imidazol-5-yl)piperazine-1-carboxylate (1.3 g, 1.85 mmol) in TFA (14 mL) was added TfOH (2.8 mL). The mixture was stirred at 70° C. for 0.5 hr. LCMS showed ~65% peak with desired mass. The mixture was concentrated. The residue was purified by reversed-phase HPLC (neutral condition) and lyophilized to afford 3-(3-methyl-2-oxo-5-(piperazin-1-yl)-2,3-dihydro-1H-benzo[d]imidazol-1-yl)piperidine-2,6-dione (480 mg, 1.38 mmol, 75.00% yield, 99% purity) as a white solid. MS(M+H)⁺=344.1.

¹H NMR (400 MHz, DMSO-d₆) δ=11.07 (s, 1H), 8.78 (s, 2H), 7.00 (d, J=8.8 Hz, 1H), 6.91 (d, J=2.3 Hz, 1H), 6.69 (dd, J=2.1, 8.6 Hz, 1H), 5.31 (dd, J=5.3, 12.9 Hz, 1H), 3.33-3.23 (m, 12H), 2.95-2.83 (m, 1H), 2.70-2.63 (m, 1H).

### Step 5. Synthesis of tert-butyl (4-(2-(4-(1-(2,6-dioxopiperidin-3-yl)-3-methyl-2-oxo-2,3-dihydro-1H-benzo[d]imidazol-5-yl)piperazin-1-yl)ethyl)piperidin-1-yl)carbamate (8)

To a solution of 3-(3-methyl-2-oxo-5-(piperazin-1-yl)-2,3-dihydro-1H-benzo[d]imidazol-1-yl)piperidine-2,6-dione (380 mg, 1.10 mmol) and tert-butyl N-[4-(2-oxoethyl)-1-piperidyl]carbamate (379.25 mg, 1.10 mmol) in DCM (10 mL) was added TEA (332.58 mg, 3.29 mmol, 457.47 μL) and 4 Å MS (5 g). After stirring at 25° C. for 0.5 hr, NaBH(OAc)₃ (696.59 mg, 3.29 mmol) was added and the resulting mixture was stirred at 25° C. for 16 hr. LCMS showed ~80% peak with desired mass. The reaction mixture was diluted with DCM (30 mL) and washed with aq. NaHCO₃ (20 mL) solution. The organic layer was dried over Na₂SO₄ and concentrated. The residue was purified by flash silica gel chromatography (ISCO®; 20 g SepaFlash® Silica Flash Column, Eluent of ~24% EtOAc/Petroleum ether, gradient @ 80 mL/min) to afford tert-butyl (4-(2-(4-(1-(2,6-dioxopiperidin-3-yl)-3-methyl-2-oxo-2,3-dihydro-1H-benzo[d]imidazol-5-yl)piperazin-1-yl)ethyl)piperidin-1-yl)carbamate (280 mg, 442.34 μmol, 40.38% yield, 90% purity) as a yellow oil. MS(M+H)⁺=570.3.

¹H NMR (400 MHz, DMSO-d₆) δ=11.08 (s, 1H), 7.87 (s, 1H), 6.95 (d, J=8.5 Hz, 1H), 6.85 (s, 1H), 6.64 (d, J=8.5 Hz, 1H), 5.30 (dd, J=5.3, 12.9 Hz, 1H), 3.32 (s, 3H), 3.15-3.05 (m, 5H), 2.93-2.80 (m, 4H), 2.76-2.57 (m, 4H), 2.50-2.41 (m, 4H), 2.40-2.30 (m, 3H), 1.67-1.63 (m, 2H), 1.39 (s, 9H), 1.31-1.20 (m, 3H).

### Step 6. Synthesis of 3-(5-(4-(2-(1-aminopiperidin-4-yl)ethyl)piperazin-1-yl)-3-methyl-2-oxo-2,3-dihydro-1H-benzo[d]imidazol-1-yl)piperidine-2,6-dione (9)

To a solution of tert-butyl N-[4-[2-[4-[1-(2,6-dioxo-3-piperidyl)-3-methyl-2-oxo-benzimidazol-5-yl]piperazin-1-yl]ethyl]-1-piperidyl]carbamate (280 mg, 442.34 μmol) in dioxane (5 mL) was added HCl/dioxane (2 M, 10 mL). The mixture was stirred at 25° C. for 16 hr. LCMS showed the starting material was consumed completely and a main peak with desired mass. The mixture was concentrated to afford 3-(5-(4-(2-(1-aminopiperidin-4-yl)ethyl)piperazin-1-yl)-3-methyl-2-oxo-2,3-dihydro-1H-benzo[d]imidazol-1-yl)piperidine-2,6-dione (320 mg, crude, 2HCl) as a white solid. MS(M+H)⁺=470.3.

### Step 7. Synthesis of N-(2-chlorophenyl)-4-((2-((4-((4-(2-(4-(1-(2,6-dioxopiperidin-3-yl)-3-methyl-2-oxo-2,3-dihydro-1H-benzo[d]imidazol-5-yl)piperazin-1-yl)ethyl)piperidin-1-yl)carbamoyl)phenyl)amino)-5-fluoropyrimidin-4-yl)amino)benzamide (Compound 106)

To a solution of 4-[[4-[4-[(2-chlorophenyl)carbamoyl]anilino]-5-fluoro-pyrimidin-2-yl]amino]benzoic acid (86.67 mg, 181.36 μmol) in DMF (1 mL) was added HATU (113.89 mg, 299.54 mol) and DIPEA (129.04 mg, 998.46 μmol, 173.91 μL). After stirring at 25° C. for 0.5 hr, 3-(5-(4-(2-(1-aminopiperidin-4-yl)ethyl)piperazin-1-yl)-3-methyl-2-oxo-2,3-dihydro-1H-benzo[d]imidazol-1-yl)piperidine-2,6-dione (130 mg, 239.63 μmol, 2HCl) was added and the resulting mixture was stirred at 25° C. for 15.5 hr. LCMS showed ~40% peak with the desired mass. The reaction mixture was diluted with EtOAc (10 mL), washed with aq. NaHCO₃ (10 mL), dried over Na₂SO₄ and concentrated. The residue was purified by prep-HPLC (TFA condition; column: Phenomenex Luna C18 150*25 mm*10 um; mobile phase: [water(TFA)-ACN]; gradient: 15%-45% B over 11 min) and lyophilized to afford N-(2-chlorophenyl)-4-[[2-[4-[[4-[2-[4-[1-(2,6-dioxo-3-piperidyl)-3-methyl-2-oxo-benzimidazol-5-yl]piperazin-1-yl]ethyl]-1-piperidyl]carbamoyl]anilino]-5-fluoro-pyrimidin-4-yl]amino]benzamide (40.3 mg, 33.35 μmol, 16.70% yield, 95.8% purity, 2TFA) as a white solid. MS(M+H)⁺=929.4.

¹H NMR (400 MHz, DMSO-d₆) δ=11.09 (s, 1H), 9.95 (s, 1H), 9.78 (s, 1H), 9.67 (s, 1H), 9.62-9.48 (m, 2H), 8.26 (d, J=3.5 Hz, 1H), 8.07-7.98 (m, 4H), 7.83-7.76 (m, 2H), 7.75-7.70 (m, 2H), 7.66 (dd, J=1.3, 7.9 Hz, 1H), 7.58 (dd, J=1.3, 8.0 Hz, 1H), 7.45-7.38 (m, 1H), 7.34-7.27 (m, 1H), 7.01 (d, J=8.8 Hz, 1H), 6.94 (d, J=1.6 Hz, 1H), 6.75-6.68 (m, 1H), 5.32 (dd, J=5.3, 12.9 Hz, 1H), 3.79 (d, J=11.6 Hz, 2H), 3.62 (d, J=10.6 Hz, 2H), 3.33 (s, 2H), 3.25-3.19 (m, 3H), 3.18-3.07 (m, 3H), 2.97 (br t, J=11.3 Hz, 2H), 2.93-2.78 (m, 3H), 2.73-2.59 (m, 2H), 2.05-1.94 (m, 1H), 1.79-1.72 (m, 2H), 1.70-1.58 (m, 2H), 1.47-1.29 (m, 3H).

### Example 107. Synthesis of N-(2-chlorophenyl)-4-((2-((4-((4-(2-(4-(3-(2,6-dioxopiperidin-3-yl)-5-fluoro-1-methyl-1H-indazol-6-yl)piperazin-1-yl)ethyl)piperidin-1-yl)carbamoyl)phenyl)amino)-5-fluoropyrimidin-4-yl)amino)benzamide (Compound 107)

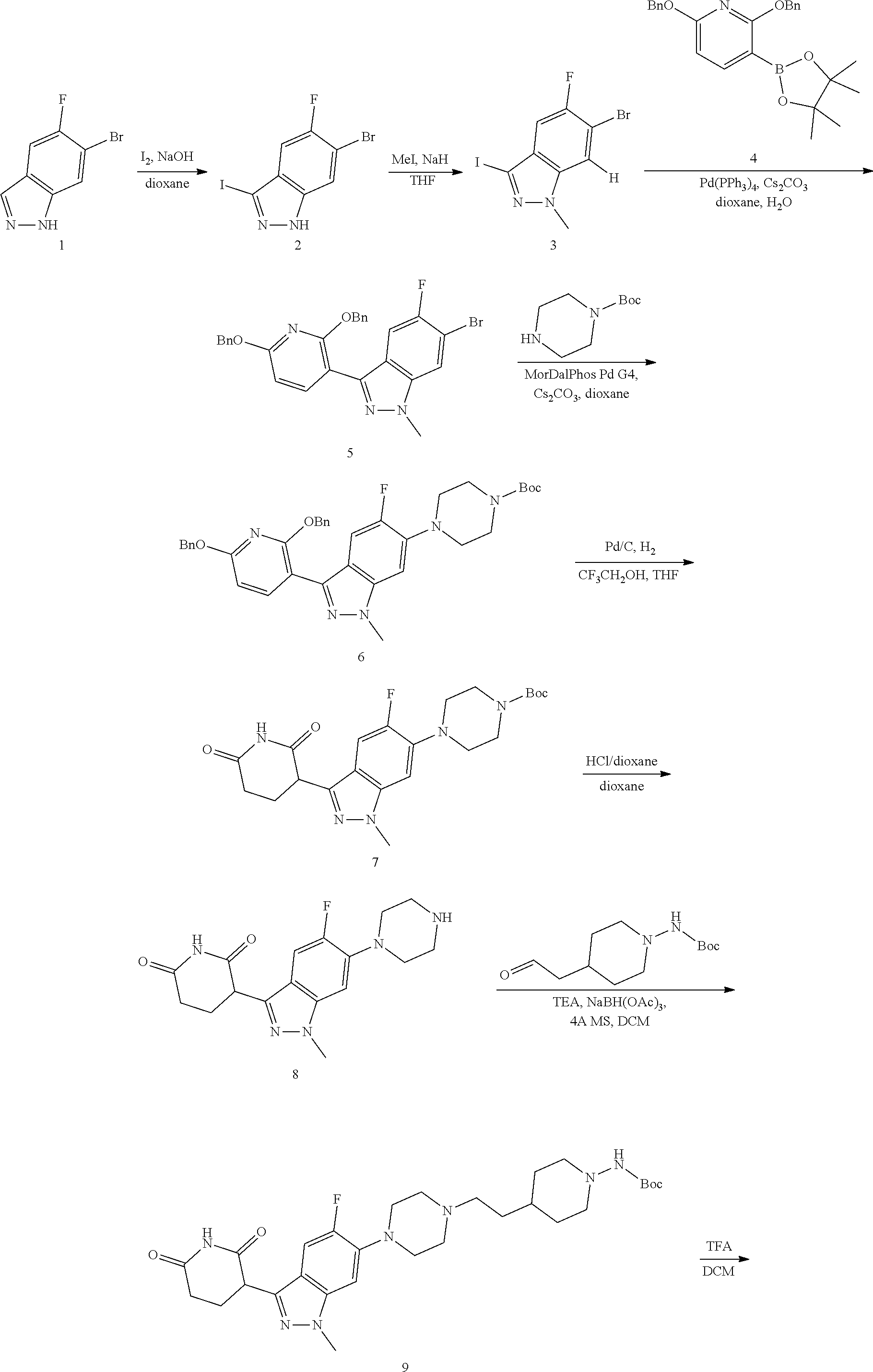

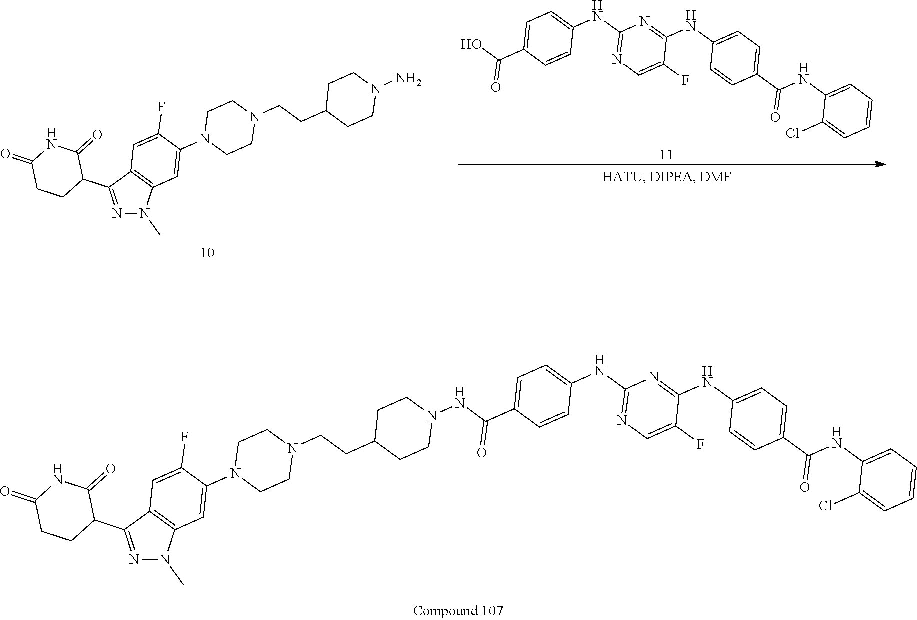 [SMILES: Cn1nc(C2CCC(=O)NC2=O)c2cc(F)c(N3CCN(CCC4CCN(N)CC4)CC3)cc21 ; O=C(O)c1ccc(Nc2ncc(F)c(Nc3ccc(C(=O)Nc4ccccc4Cl)cc3)n2)cc1 ; "11" ; "10" ; Cn1nc(C2CCC(=O)NC2=O)c2cc(F)c(N3CCN(CCC4CCN(NC(=O)c5ccc(Nc6ncc(F)c(Nc7ccc(C(=O)Nc8ccccc8Cl)cc7)n6)cc5)CC4)CC3)cc21]

### Step 1. Synthesis of 6-bromo-5-fluoro-3-iodo-1H-indazole (2)

To a solution of 6-bromo-5-fluoro-1H-indazole (1.7 g, 7.91 mmol) in dioxane (30 mL) were added I₂ (4.01 g, 15.81 mmol, 3.19 mL) and NaOH (3 M, 2.64 mL). The mixture was stirred at 20° C. for 2 hr. LCMS showed a main peak with desired mass. The reaction mixture was quenched with 20 mL of aqueous Na₂S₂O₃ and 20 mL of aqueous NaHCO₃ solution. The resulting mixture was extracted with EtOAc (60 mL×3). The combined organic phase was concentrated under reduced pressure to afford 6-bromo-5-fluoro-3-iodo-1H-indazole (2.7 g, 7.44 mmol, 94.16% yield, 94% purity) as black brown solid.

¹H NMR (400 MHz, CDCl₃) δ=11.58-10.92 (m, 1H), 7.81 (br d, J=5.1 Hz, 1H), 7.25 (d, J=7.8 Hz, 1H).

### Step 2. Synthesis of 6-bromo-5-fluoro-3-iodo-1-methyl-1H-indazole (3)

To a solution of 6-bromo-5-fluoro-3-iodo-1H-indazole (2.4 g, 7.04 mmol) in THF (40 mL) was added NaH (422.39 mg, 10.56 mmol, 60% purity) at 0° C. under N₂ atmosphere. After stirring at 0° C. for 15 min under N₂ atmosphere, MeI (2.00 g, 14.08 mmol) was added and the resulting mixture was stirred at 20° C. for 90 min. LCMS showed the starting material was consumed completely and desired mass was detected. The reaction mixture was quench with 30 mL of aqueous NH₄Cl solution and extracted with EtOAc (30 mL×3). The combined organic phase was concentrated under reduced pressure to afford a residue. The residue was purified by flash silica gel chromatography (20 g SepaFlash® Silica Flash Column, Eluent of 0-3% EtOAc/Petroleum ether, gradient @ 60 mL/min) to afford 6-bromo-5-fluoro-3-iodo-1-methyl-indazole (1.58 g, 4.41 mmol, 62.60% yield, 99% purity) as white solid.

¹H NMR (400 MHz, CDCl₃) δ=7.71-7.61 (m, 1H), 7.20 (td, J=2.9, 7.8 Hz, 1H), 4.08 (s, 3H).

### Step 3. Synthesis of 3-(2,6-bis(benzyloxy)pyridin-3-yl)-6-bromo-5-fluoro-1-methyl-1H-indazole (5)

A mixture of 6-bromo-5-fluoro-3-iodo-1-methyl-indazole (1.58 g, 4.45 mmol), 2,6-dibenzyloxy-3-(4,4,5,5-tetramethyl-1,3,2-dioxaborolan-2-yl)pyridine (1.86 g, 4.45 mmol), Cs₂CO₃ (4.35 g, 13.35 mmol) and Pd(PPh₃)₄ (514.39 mg, 445.14 μmol) in dioxane (32 mL) and H₂O (8 mL) was degassed and purged with N₂ for 3 times, and then the mixture was stirred at 100° C. for 16 hr under N₂ atmosphere. LCMS showed a peak (5%) with desired mass. To the mixture was added 2,6-dibenzyloxy-3-(4,4,5,5-tetramethyl-1,3,2-dioxaborolan-2-yl)pyridine (928.79 mg, 2.23 mmol) and Pd(PPh₃)₄ (514.39 mg, 445.14 μmol). The resulting mixture was degassed and purged with N₂ for 3 times, and then the mixture was stirred at 100° C. for 16 hr under N₂ atmosphere. LCMS showed a peak (24%) with desired mass. The mixture was diluted with 30 mL of water and extracted with EtOAc (30 mL×3). The combined organic phase was concentrated under reduced pressure. The residue was purified by flash silica gel chromatography (12 g SepaFlash® Silica Flash Column, Eluent of 0~20% EtOAc/Petroleum ether, gradient @ 60 mL/min) to afford 6-bromo-3-(2,6-dibenzyloxy-3-pyridyl)-5-fluoro-1-methyl-indazole (1.8 g, 3.02 mmol, 67.87% yield, 87% purity) as yellow solid. MS(M+H)⁺=520.0.

¹H NMR (400 MHz, CDCl₃) δ=7.91 (d, J=8.2 Hz, 1H), 7.58 (d, J=5.4 Hz, 1H), 7.49-7.28 (m, 11H), 6.53 (d, J=8.2 Hz, 1H), 5.47-5.43 (m, 4H), 4.06 (s, 3H).

### Step 4. Synthesis of tert-butyl 4-(3-(2,6-bis(benzyloxy)pyridin-3-yl)-5-fluoro-1-methyl-1H-indazol-6-yl)piperazine-1-carboxylate (6)

To a solution of 6-bromo-3-(2,6-dibenzyloxy-3-pyridyl)-5-fluoro-1-methyl-indazole (700 mg, 1.35 mmol) and tert-butyl piperazine-1-carboxylate (251.51 mg, 1.35 mmol) in dioxane (15 mL) were added MorDalPhos Pd G4 (57.42 mg, 67.52 μmol) and Cs₂ CO₃ (1.32 g, 4.05 mmol). The resulting mixture was stirred at 100° C. for 16 hr. LCMS showed 40% of desired compound was detected. The residue was diluted with H₂O (80 mL) and extracted with solvent EtOAc (100 mL×3). The combined organic layers were washed with brine (150 mL), dried over Na₂SO₄, filtered and concentrated under reduced pressure. The residue was purified by flash silica gel chromatography (ISCO®; 12 g SepaFlash® Silica Flash Column, Eluent of 0~50% EtOAc/Petroleum ether, gradient @ 50 mL/min) to afford tert-butyl 4-[3-(2,6-dibenzyloxy-3-pyridyl)-5-fluoro-1-methyl-indazol-6-yl]piperazine-1-carboxy late (560 mg, 808.06 μmol, 59.84% yield, 90% purity) as a yellow solid. MS(M+H)⁺=624.4.

### Step 5. Synthesis of tert-butyl 4-(3-(2,6-dioxopiperidin-3-yl)-5-fluoro-1-methyl-1H-indazol-6-yl)piperazine-1-carboxylate (7)

To a solution of tert-butyl 4-[3-(2,6-dibenzyloxy-3-pyridyl)-5-fluoro-1-methyl-indazol-6-yl]piperazine-1-carboxylate (510 mg, 735.91 μmol) in CF₃CH₂OH (10 mL) was added Pd/C (78.32 mg, 73.59 μmol, 10% purity). The mixture was stirred at 25° C. for 7 hr under H₂ (50 Psi). LCMS showed 8% of starting material remained and 67% of intermediate state. The mixture was stirred at 25° C. for 12 hr. LCMS showed 77% peak of intermediate state. Another portion of Pd/C (78.32 mg, 73.59 μmol, 10% purity) and THF (20 mL) was added. The mixture was stirred at 25° C. for 12 hr under H₂ (50 Psi). LCMS showed 56% peak of desired compound was detected. The reaction mixture was filtered and concentrated under reduced pressure. The residue was purified by flash silica gel chromatography (ISCO®; 12 g SepaFlash® Silica Flash Column, Eluent of 0-10% EtOAc/Petroleum ether gradient @ 45 mL/min). The product was further purified by prep-HPLC (column: Phenomenex luna C18 150*25 mm*10 um; mobile phase: [water (FA)-ACN]; gradient: 43%-63% B over 10 min) and lyophilized to afford tert-butyl 4-[3-(2,6-dioxo-3-piperidyl)-5-fluoro-1-methyl-indazol-6-yl]piperazine-1-carboxylate (60 mg, 115.83 μmol, 15.74% yield, 86% purity) and tert-butyl 4-[3-(2,6-dioxo-3-piperidyl)-5-fluoro-1-methyl-indazol-6-yl]piperazine-1-carboxylate (62 mg, 139.17 μmol, 18.91% yield, 100% purity) as a white solid. MS(M+H)⁺=446.2.

### Step 6. Synthesis of 3-(5-fluoro-1-methyl-6-(piperazin-1-yl)-1H-indazol-3-yl)piperidine-2,6-dione (8)

To a solution of tert-butyl 4-[3-(2,6-dioxo-3-piperidyl)-5-fluoro-1-methyl-indazol-6-yl]piperazine-1-carboxylate (120 mg, 269.37 μmol) in dioxane (10 mL) was added HCl/dioxane (2 M, 10 mL). The mixture was stirred at 25° C. for 0.5 hr. LCMS showed a main peak with desired mass. The mixture was concentrated under reduced pressure to afford 3-(5-fluoro-1-methyl-6-piperazin-1-yl-indazol-3-yl)piperidine-2,6-dione (150 mg, crude, HCl) as a white solid. MS(M+H)⁺=346.2.

### Step 7. Synthesis of tert-butyl (4-(2-(4-(3-(2,6-dioxopiperidin-3-yl)-5-fluoro-1-methyl-1H-indazol-6-yl)piperazin-1-yl)ethyl)piperidin-1-yl)carbamate (9)

To a solution of 3-(5-fluoro-1-methyl-6-piperazin-1-yl-indazol-3-yl)piperidine-2,6-dione (150 mg, 392.84 μmol, HCl) and tert-butyl N-[4-(2-oxoethyl)-1-piperidyl]carbamate (114.23 mg, 471.41 μmol) in DCM (10 mL) were added TEA (79.50 mg, 785.69 μmol) and 4 Å MS (100 mg). After stirring at 25° C. for 1 hr, NaBH(OAc)₃ (124.89 mg, 589.27 μmol) was added and the resulting mixture was stirred at 25° C. for 14 hr. LCMS showed the starting material was consumed completely and a 82% peak with desired mass. The reaction mixture was diluted with H₂O (50 mL) and the layers were separated. The aqueous layer was extracted with EtOAc (60 mL×2). The combined organic layers were dried over Na₂SO₄, filtered and concentrated. The residue was purified by flash silica gel chromatography (ISCO®; 12 g SepaFlash® Silica Flash Column, Eluent of 0~50% EtOAc/Petroleum ether) to afford tert-butyl N-[4-[2-[4-[3-(2,6-dioxo-3-piperidyl)-5-fluoro-1-methyl-indazol-6-yl]piperazin-1-yl]ethyl]-1-piperidyl]carbamate (120 mg, 201.51 μmol, 51.30% yield, 96% purity) as a white solid. MS (M+H)⁺=572.3.

### Step 8. Synthesis of 3-(6-(4-(2-(1-aminopiperidin-4-yl)ethyl)piperazin-1-yl-5-fluoro-1-methyl-1H-indazol-3-yl)piperidine-2,6-dione (10)

To a solution of tert-butyl N-[4-[2-[4-[3-(2,6-dioxo-3-piperidyl)-5-fluoro-1-methyl-indazol-6-yl]piperazin-1-yl]ethyl]-1-piperidyl]carbamate (100 mg, 174.92 μmol) in DCM (5 mL) was added TFA (1.54 g, 13.46 mmol, 1 mL). The mixture was stirred at 25° C. for 2 hr. LCMS the starting material was consumed completely and the desired mass. The reaction mixture was concentrated under reduced pressure to afford 3-[6-[4-[2-(1-amino-4-piperidyl)ethyl]piperazin-1-yl]-5-fluoro-1-methyl-indazol-3-yl]piperidine-2,6-dione (102 mg, crude, TFA) as a brown oil. MS(M+H)⁺=472.3.

### Step 9. Synthesis of N-(2-chlorophenyl)-4-((2-((4-((4-(2-(4-(3-(2,6-dioxopiperidin-3-yl)-5-fluoro-1-methyl-1H-indazol-6-yl)piperazin-1-yl)ethyl)piperidin-1-yl)carbamoyl)phenyl)amino)-5-fluoro pyrimidin-4-yl)amino)benzamide (Compound 107)

To a solution of 4-[[4-[4-[(2-chlorophenyl)carbamoyl]anilino]-5-fluoro-pyrimidin-2-yl]amino]benzoic acid (83.24 mg, 174.18 μmol) in DMF (2 mL) was added HATU (99.34 mg, 261.27 μmol) and DIPEA (67.54 mg, 522.55 μmol). After stirring at 25° C. for 1 hr, 3-[6-[4-[2-(1-amino-4-piperidyl)ethyl]piperazin-1-yl]-5-fluoro-1-methyl-indazol-3-yl]piperidine-2,6-dione (102 mg, 174.18 μmol, TFA) was added and the resulting mixture was stirred at 25° C. for 12 hr. LCMS showed 77% peak of desired mass. The reaction mixture was diluted with H₂O (10 mL) and extracted with EtOAc (5 mL×3). The combined organic layers were dried over Na₂SO₄, filtered and concentrated. The residue was purified by prep-HPLC (column: Phenomenex luna C18 150*25 mm*10 um; mobile phase: [water (TFA)-ACN]; gradient: 24%-54% B over 10 min) and the eluent was lyophilized to afford the crude product. The crude product was dissolved with H₂O (5 mL) and ACN (1 mL), then adjust pH to 8 with saturated NaHCO₃ solution. The mixture was filtered. The filter cake was collected and dried to afford N-(2-chlorophenyl)-4-[[2-[4-[[4-[2-[4-[3-(2,6-dioxo-3-piperidyl)-5-fluoro-1-methyl-indazol-6-yl]piperazin-1-yl]ethyl]-1-piperidyl]carbamoyl]anilino]-5-fluoro-pyrimidin-4-yl]amino]benzamide (19.3 mg, 18.23 μmol, 58.57% yield, 88% purity) as a white solid. MS(M+H)⁺=931.5.

¹H NMR (400 MHz, DMSO-d₆) δ=10.04-9.76 (m, 2H), 9.54 (br s, 1H), 9.12 (br s, 1H), 8.20 (br d, J=3.2 Hz, 1H), 8.03-7.96 (m, 4H), 7.79-7.74 (m, 2H), 7.71-7.64 (m, 3H), 7.56 (dd, J=1.1, 7.9 Hz, 2H), 7.45 (d, J=12.7 Hz, 1H), 7.42-7.35 (m, 1H), 7.31-7.25 (m, 1H), 7.11 (d, J=7.1 Hz, 1H), 4.27 (dd, J=5.0, 9.5 Hz, 1H), 3.95 (s, 3H), 3.13-3.06 (m, 4H), 2.98 (br d, J=9.0 Hz, 2H), 2.75-2.66 (m, 2H), 2.65-2.54 (m, 6H), 2.42-2.32 (m, 3H), 2.19-2.10 (m, 1H), 1.73-1.68 (m, 2H), 1.48-1.38 (m, 2H), 1.34-1.25 (m, 3H).

### Example 108. Synthesis of (R)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (Compound 108)

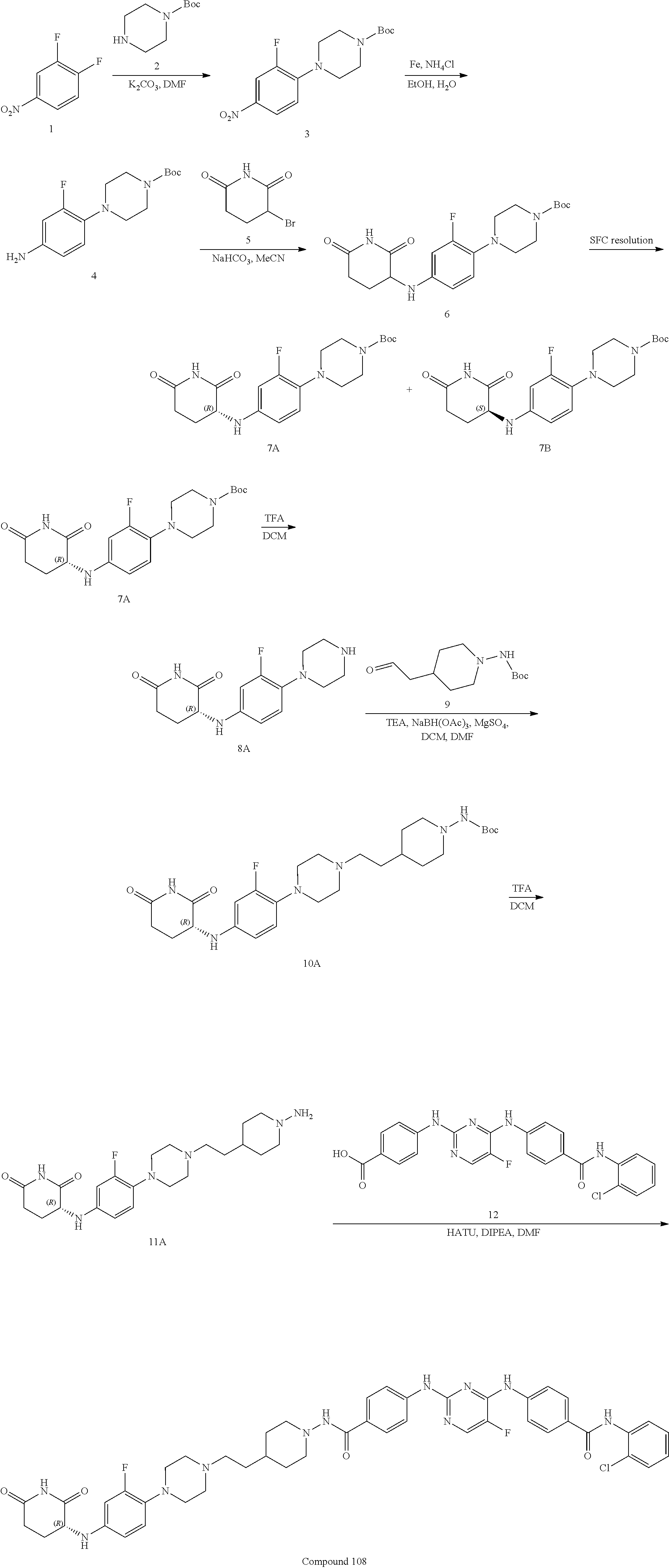

### Step 1. Synthesis of tert-butyl 4-(2-fluoro-4-nitrophenyl)piperazine-1-carboxylate (3)

To a solution of 1,2-difluoro-4-nitrobenzene (1 g, 6.29 mmol, 695.89 μL) and tert-butyl piperazine-1-carboxylate (1.17 g, 6.29 mmol) in DMF (10 mL) was added K₂CO₃ (1.74 g, 12.57 mmol). The mixture was stirred at 80° C. for 16 h. LCMS showed 70% peak of the desired mass. The reaction mixture was diluted with water (40 mL) and extracted with EtOAc (40 mL×3). The combined organic phase was washed with brine (30 mL×3), dried over Na₂SO₄ and concentrated under reduced pressure to afford a residue. The residue was triturated with EtOAc (20 mL) and ACN (2 mL) to afford tert-butyl 4-(2-fluoro-4-nitrophenyl)piperazine-1-carboxylate (1.68 g, 5.16 mmol, 82.15% yield, 100% purity) as a yellow solid. MS(M−55)+=270.0.

¹H NMR (400 MHz, CDCl₃) δ=8.04-7.96 (m, 1H), 7.96-7.87 (m, 1H), 6.92 (t, J=8.7 Hz, 1H), 3.68-3.56 (m, 4H), 3.32-3.18 (m, 4H), 1.49 (s, 9H).

### Step 2. Synthesis of tert-butyl 4-(4-amino-2-fluorophenyl)piperazine-1-carboxylate

To a mixture of tert-butyl 4-(2-fluoro-4-nitrophenyl)piperazine-1-carboxylate (1.5 g, 4.61 mmol) and NH₄Cl (1.23 g, 23.05 mmol) in EtOH (15 mL) and H₂O (15 mL) was added Fe (772.44 mg, 13.83 mmol). The mixture was stirred at 80° C. for 1 hr. LCMS showed a peak (68%) with desired mass. The reaction mixture was filtered through celite pad and the filtrate was concentrated under reduced pressure. And then the crude was diluted with aqueous NaHCO₃ solution (30 mL) and the mixture was extracted with EtOAc (40 mL×2). The combined organic phase was dried over Na₂SO₄ and concentrated under reduced pressure to afford tert-butyl 4-(4-amino-2-fluorophenyl)piperazine-1-carboxylate (1.3 g, 2.73 mmol, 59.19% yield, 62% purity) as a yellow oil. MS(M+H)⁺=296.1.

### Step 3. Synthesis of tert-butyl (R)-4-(4-((2,6-dioxopiperidin-3-yl)amino)-2-fluorophenyl)piperazine-1-carboxylate (7A) and tert-butyl (S)-4-(4-((2,6-dioxopiperidin-3-yl)amino)-2-fluorophenyl)piperazine-1-carboxylate (7)

To a solution of tert-butyl 4-(4-amino-2-fluorophenyl)piperazine-1-carboxylate (1.3 g, 4.40 mmol) in MeCN (7.5 mL) was added NaHCO₃ (1.85 g, 22.01 mmol, 856.35 μL) and 3-bromopiperidine-2,6-dione (2.11 g, 11.00 mmol). The mixture was stirred at 80° C. for 16 hr. LCMS showed 29% peak of the desired mass. The reaction mixture was concentrated under reduced pressure and then purified by flash silica gel chromatography (12 g SepaFlash® Silica Flash Column, Eluent of 0~50% EtOAc/Petroleum ether gradient @ 60 mL/min) to afford the racemate tert-butyl 4-(4-((2,6-dioxopiperidin-3-yl)amino)-2-fluorophenyl)piperazine-1-carboxylate (1.4 g). The racemate product was purified by SFC (column: DAICEL CHIRALPAK IG (250 mm*30 mm, 10 um); mobile phase: [CO₂-i-PrOH/ACN]; B %: 50%, isocratic elution mode) and then concentrated to afford tert-butyl (S)-4-(4-((2,6-dioxopiperidin-3-yl)amino)-2-fluorophenyl)piperazine-1-carboxylate (610 mg, 1.47 mmol, 33.42% yield, 98% purity) and tert-butyl (R)-4-(4-((2,6-dioxopiperidin-3-yl)amino)-2-fluorophenyl)piperazine-1-carboxylate (640 mg, 1.48 mmol, 33.63% yield, 94% purity) as a blue solid. MS(M+H)⁺=407.1.

### Step 4. Synthesis of (R)-3-((3-fluoro-4-(piperazin-1-yl)phenyl)amino)piperidine-2,6-dione (8A)

To a solution of tert-butyl (R)-4-(4-((2,6-dioxopiperidin-3-yl)amino)-2-fluorophenyl)piperazine-1-carboxylate (0.2 g, 492.07 μmol) in DCM (2 mL) was added TFA (921.00 mg, 8.08 mmol, 0.6 mL) at 0° C. and the mixture was stirred at 0° C. for 1 h. LCMS showed 94% peak of the desired mass. The mixture was concentrated under reduced pressure to afford (R)-3-((3-fluoro-4-(piperazin-1-yl)phenyl)amino)piperidine-2,6-dione (0.21 g, crude, TFA) as a blue oil. MS(M+H)⁺=307.2.

### Step 5. Synthesis of tert-butyl (R)-(4-(2-(4-(4-((2,6-dioxopiperidin-3-yl)amino)-2-fluorophenyl)piperazin-1-yl)ethyl) piperidin-1-yl)carbamate (10A)

To a solution of (R)-3-((3-fluoro-4-(piperazin-1-yl)phenyl)amino)piperidine-2,6-dione (160 mg, 380.63 mol, TFA) in DCM (3 mL) were added TEA (203.56 mg, 2.01 mmol, 280 μL) and MgSO₄ (137.45 mg, 1.14 mmol) and then followed by a solution of tert-butyl (4-(2-oxoethyl)piperidin-1-yl)carbamate (150.00 mg, 619.03 μmol) in DMF (3 mL) and the mixture was stirred at 0° C. for 30 min. NaBH(OAc)₃ (161.34 mg, 761.26 μmol) was added and the mixture was stirred at 0° C. for 1 h. LCMS showed 15% peak of (R)-3-((3-fluoro-4-(piperazin-1-yl)phenyl)amino)piperidine-2,6-dione remained and 61% peak of the desired mass. Then a solution of tert-butyl (4-(2-oxoethyl)piperidin-1-yl)carbamate (50 mg, 206.34 μmol) in DMF (1 mL) was added and the mixture was stirred at 0° C. for 0.5 h. LCMS showed 63% of the desired mass. The mixture was diluted with water (10 mL) and extracted with EtOAc (10 mL×3). Then the aqueous phase was basified to pH=8 with saturated NaHCO₃ solution and then extracted with DCM (20 mL×3). Two parts of the organic phases were combined and dried over Na₂SO₄ and filtered. The filtrate was concentrated under reduced pressure and then purified by flash silica gel chromatography (12 g SepaFlash® Silica Flash Column, Eluent of 10-14% EtOH/EtOAc gradient @ 100 mL/min) to afford tert-butyl (R)-(4-(2-(4-(4-((2,6-dioxopiperidin-3-yl)amino)-2-fluorophenyl)piperazin-1-yl)ethyl) piperidin-1-yl)carbamate (160 mg, 258.33 μmol, 67.87% yield, 86% purity) as a yellow solid. MS(M+H)⁺=533.3.

### Step 6. Synthesis of (R)-3-((4-(4-(2-(1-aminopiperidin-4-yl)ethyl)piperazin-1-yl)-3-fluorophenyl) aminol)piperidine-2,6-dione (11A)

To a solution of tert-butyl (R)-(4-(2-(4-(4-((2,6-dioxopiperidin-3-yl)amino)-2-fluorophenyl)piperazin-1-yl)ethyl) piperidin-1-yl)carbamate (160 mg, 258.33 μmol) in DCM (1.5 mL) was added TFA (614.00 mg, 5.38 mmol, 0.4 mL) and the mixture was stirred at 25° C. for 1 h. LCMS showed 82% peak with the desired mass. The mixture was concentrated under reduced pressure to afford (R)-3-((4-(4-(2-(1-aminopiperidin-4-yl)ethyl)piperazin-1-yl)-3-fluorophenyl) amino)piperidine-2,6-dione (170 mg, crude, TFA) as a yellow oil. MS(M+H)⁺=433.3.

### Step 7. Synthesis of (R)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (Compound 108)

To a mixture of 4-((4-((4-((2-chlorophenyl)carbamoyl)phenyl)amino)-5-fluoropyrimidin-2-yl)amino)benzoic acid (140 mg, 292.96 μmol) and HATU (133.67 mg, 351.56 μmol) in DMF (2 mL) was added DIPEA (371.00 mg, 2.87 mmol, 0.5 mL) and the mixture was stirred at 25° C. for 15 min. Then a solution of (R)-3-((4-(4-(2-(1-aminopiperidin-4-yl)ethyl)piperazin-1-yl)-3-fluorophenyl)amino)piperidine-2,6-dione (170 mg, 311.04 μmol, TFA) in DMF (2 mL) (0° C.) was added at 0° C. and the mixture was stirred at 0° C. for 30 min. LCMS showed 44% peak of the desired mass. The mixture was poured into water (10 mL). The mixture was filtered and washed with water (10 mL). The filter cake was collected and purified by prep-HPLC (column: Phenomenex luna C18 150*25 mm*10 um; mobile phase: [water (TFA)-ACN]; gradient: 21%-51% B over 10 min). The fraction was lyophilized to afford (R)—N-(2-chlorophenyl)-4-((2-((4-((4-(2-(4-(4-((2,6-dioxopiperidin-3-yl)amino)-2-fluorophenyl)piperazin-1-yl)ethyl)piperidin-1-yl)carbamoyl)phenyl)amino)-5-fluoropyrimidin-4-yl)amino)benzamide (92 mg, 73.78 μmol, 25.18% yield, 99% purity, 3TFA) as a yellow solid. MS(M+H)⁺=892.4.

¹H NMR (400 MHz, DMSO-d₆) δ=10.79 (s, 1H), 10.01-9.74 (m, 3H), 9.73-9.66 (m, 1H), 9.65-9.47 (m, 1H), 8.26 (d, J=3.5 Hz, 1H), 8.06-7.96 (m, 4H), 7.84-7.77 (m, 2H), 7.76-7.70 (m, 2H), 7.65 (dd, J=1.3, 7.9 Hz, 1H), 7.57 (dd, J=1.3, 8.0 Hz, 1H), 7.40 (dt, J=1.3, 7.7 Hz, 1H), 7.33-7.26 (m, 1H), 6.89 (t, J=9.3 Hz, 1H), 6.58-6.52 (m, 1H), 6.46 (dd, J=1.8, 8.8 Hz, 1H), 4.29 (br dd, J=4.6, 11.5 Hz, 1H), 3.60-3.53 (m, 2H), 3.31-3.10 (m, 8H), 3.01-2.81 (m, 4H), 2.78-2.67 (m, 1H), 2.62-2.54 (m, 1H), 2.13-2.03 (m, 1H), 1.93-1.80 (m, 1H), 1.79-1.71 (m, 2H), 1.68-1.58 (m, 2H), 1.48-1.34 (m, 3H).

SFC Method details: “Column: Chiralpak AS-3 50×4.6 mm I.D., 3 um Mobile phase: Phase A for CO₂, and Phase B for IPA+ACN (0.05% DEA); Gradient elution: 60% IPA+ACN (0.05% DEA) in CO₂ Flow rate: 3 mL/min; Detector: PDA Column Temp: 35 C; Back Pressure: 100 Bar”

### Example 109. Synthesis of N-(4-(2-(4-(4-((2,6-dioxopiperidin-3-yl)amino)-2,5-difluorophenyl)piperazin-1-yl)ethyl)piperidin-1-yl)-2-fluoro-4-((5-fluoro-4-((4-(2-fluorobenzamido)phenyl)amino)pyrimidin-2-yl)amino)benzamide (Compound 109)

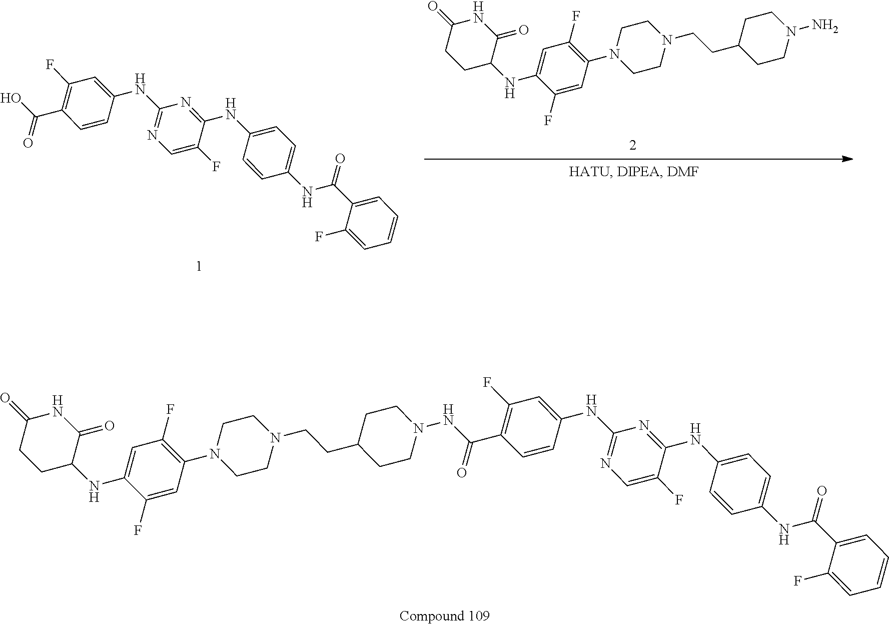 [SMILES: O=C(O)c1ccc(Nc2ncc(F)c(Nc3ccc(NC(=O)c4ccccc4F)cc3)n2)cc1F ; NN1CCC(CCN2CCN(c3cc(F)c(NC4CCC(=O)NC4=O)cc3F)CC2)CC1 ; "1" ; O=C1CCC(Nc2cc(F)c(N3CCN(CCC4CCN(NC(=O)c5ccc(Nc6ncc(F)c(Nc7ccc(NC(=O)c8ccccc8F)cc7)n6)cc5F)CC4)CC3)cc2F)C(=O)N1]

### Step 1. Synthesis of N-(4-(2-(4-(4-((2,6-dioxopiperidin-3-yl)amino)-2,5-difluorophenyl)piperazin-1-yl)ethyl)piperidin-1-yl)-2-fluoro-4-((5-fluoro-4-((4-(2-fluorobenzamido)phenyl)amino)pyrimidin-2-yl)amino)benzamide (Compound 109)

To a solution of 2-fluoro-4-((5-fluoro-4-((4-(2-fluorobenzamido)phenyl)amino)pyrimidin-2-yl)amino)benzoic acid (140 mg, 292.03 μmol) in DMF (2 mL) was added HATU (166.56 mg, 438.04 μmol) and DIPEA (377.41 mg, 2.92 mmol, 508.64 μL) and 3-((4-(4-(2-(1-aminopiperidin-4-yl)ethyl)piperazin-1-yl)-2,5-difluorophenyl)amino)piperidine-2,6-dione (247.29 mg, 438.04 μmol, TFA salt). The mixture was stirred at 25° C. for 2 hr. LCMS showed 3-((4-(4-(2-(1-aminopiperidin-4-yl)ethyl)piperazin-1-yl)-2,5-difluorophenyl)amino)piperidine-2,6-dione was consumed completely and ~27% of desired compound was detected. The reaction mixture was poured into water (10 mL) and filtered. The filter cake was purified by prep-HPLC (column: Phenomenex luna C18 150*25 mm*10 um; mobile phase: [water(TFA)-ACN]; gradient: 22%-52% B over 10 min) and lyophilized to afford N-(4-(2-(4-(4-((2,6-dioxopiperidin-3-yl)amino)-2,5-difluorophenyl)piperazin-1-yl)ethyl)piperidin-1-yl)-2-fluoro-4-((5-fluoro-4-((4-(2-fluorobenzamido)phenyl)amino)pyrimidin-2-yl)amino)benzamide (42.6 mg, 34.60 μmol, 11.85% yield, 92.6% purity, 2TFA salt) as a yellow solid. MS(M+H)⁺=912.4.

¹H NMR (400 MHz, DMSO-d₆) δ=10.80 (s, 1H), 9.71 (s, 1H), 9.53 (s, 1H), 9.42-9.31 (m, 1H) 9.31-9.21 (m, 1H), 8.18 (d, J=3.5 Hz, 1H), 7.81 (br d, J=14.1 Hz, 1H), 7.77-7.72 (s, 4H), 7.68 (br t, J=7.0 Hz, 1H), 7.62-7.56 (m, 1H), 7.44 (br d, J=4.3 Hz, 2H), 7.38-7.31 (m, 2H), 6.97 (dd, J=7.8, 13.0 Hz, 1H), 6.78 (br dd, J=8.1, 14.5 Hz, 1H), 4.43-4.32 (m, 1H), 3.37-3.27 (m, 4H), 3.24-3.14 (m, 4H), 3.13-3.03 (m, 3H), 2.99-2.90 (m, 2H), 2.76-2.64 (m, 3H), 2.08-1.98 (m, 2H), 1.81-1.67 (m, 2H), 1.66-1.53 (m, 2H), 1.40-1.30 (m, 3H).

### Example 110. Synthesis of N-(2-chlorophenyl)-4-[[2-[4-[4-[2-[4-[4-(2,6-dioxo-3-piperidyl)phenyl]piperazin-1-yl]ethyl]piperidine-1-carbonyl]anilino]-5-fluoro-pyrimidin-4-yl]amino]benzamide (Compound 110)

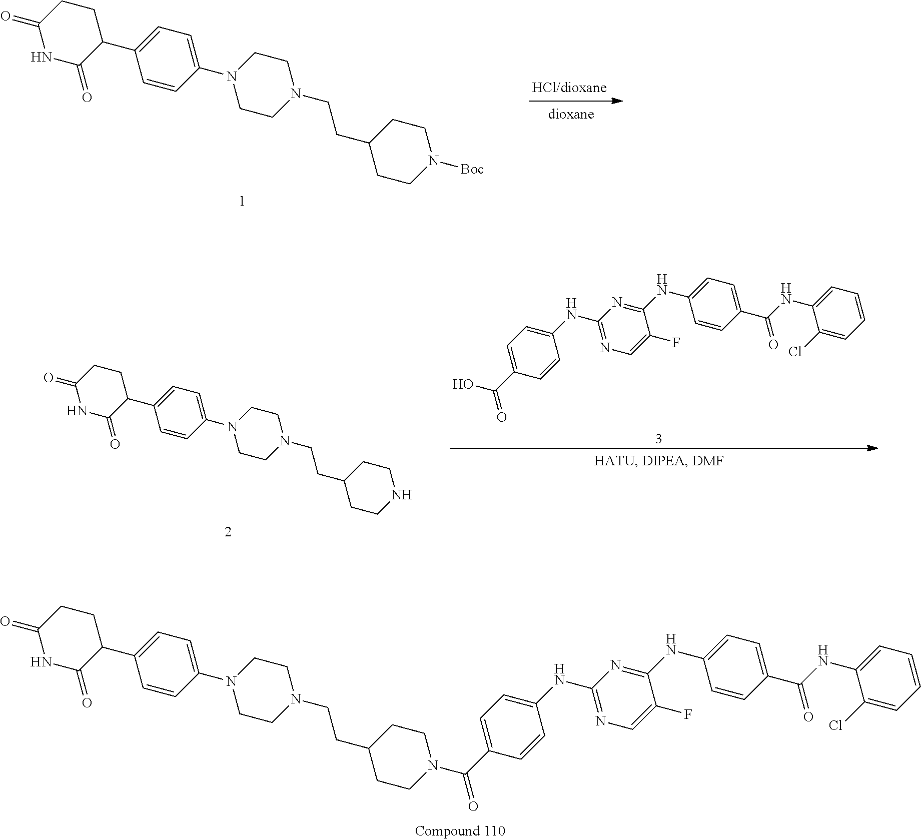

### Step 1. Synthesis of 3-[4-[4-[2-(4-piperidyl)ethyl]piperazin-1-yl]phenyl]piperidine-2,6-dione (2)

To a suspension of tert-butyl 4-[2-[4-[4-(2,6-dioxo-3-piperidyl)phenyl]piperazin-1-yl]ethyl]piperidine-1-carboxylate (140 mg, 288.88 μmol) in dioxane (2 mL) was added HCl/dioxane (2 M, 6 mL). The mixture was stirred at 25° C. for 2 hours. LCMS showed a main peak with desired mass. The mixture was concentrated in vacuum to afford 3-[4-[4-[2-(4-piperidyl)ethyl]piperazin-1-yl]phenyl]piperidine-2,6-dione (121.6 mg, 288.85 μmol, 99.99% yield, HCl) as an off-white solid. MS(M+H)⁺=385.3.

### Step 2. Synthesis of N-(2-chlorophenyl)-4-[[2-[4-[4-[2-[4-[4-(2,6-dioxo-3-piperidyl)phenyl]piperazin-1-yl]ethyl]piperidine-1-carbonyl]anilino]-5-fluoro-pyrimidin-4-yl]amino]benzamide (Compound 110)

To a suspension of 3-[4-[4-[2-(4-piperidyl)ethyl]piperazin-1-yl]phenyl]piperidine-2,6-dione (122 mg, 288.85 μmol, HCl) and 4-[[4-[4-[(2-chlorophenyl)carbamoyl]anilino]-5-fluoro-pyrimidin-2-yl]amino]benzoic acid (104 mg, 216.64 μmol) in DMF (4 mL) was added HATU (132 mg, 346.62 μmol) and DIPEA (187 mg, 1.44 mmol). The mixture was stirred at 25° C. for 2 hours. LCMS showed the starting material was consumed completely ~59% peak of desired compound was detected. The reaction mixture was poured into water (10 mL) and filtered. The filter cake was purified by prep-HPLC (TFA condition, column: Phenomenex luna C18 150*25 mm*10 um; mobile phase: [water(TFA)-ACN]; gradient: 21%-51% B over 10 min) and lyophilized to afford N-(2-chlorophenyl)-4-[[2-[4-[4-[2-[4-[4-(2,6-dioxo-3-piperidyl)phenyl]piperazin-1-yl]ethyl]piperidine-1-carbonyl]anilino]-5-fluoro-pyrimidin-4-yl]amino]benzamide (58.1 mg, 60.38 μmol, 6.97% yield, 99.6% purity, TFA) as a white solid. MS(M+H)⁺=844.4.

¹H NMR (400 MHz, DMSO-d₆) δ=10.79 (s, 1H), 9.95 (s, 1H), 9.77 (s, 1H), 9.75 (s, 1H), 9.58 (s, 1H), 9.56-9.45 (m, 1H), 8.23 (d, J=3.6 Hz, 1H), 8.05-7.96 (m, 4H), 7.74 (d, J=8.8 Hz, 2H), 7.65 (dd, J=8.0, 1.2 Hz, 1H), 7.56 (dd, J=8.0, 1.2 Hz, 1H), 7.45-7.37 (m, 1H), 7.35-7.27 (m, 3H), 7.12 (d, J=8.0 Hz, 2H), 6.97 (d, J=8.8 Hz, 2H), 3.85-3.72 (m, 4H), 3.60-3.49 (m, 2H), 3.27-3.14 (m, 2H), 3.14-3.04 (m, 2H), 3.01-2.88 (m, 3H), 2.70-2.59 (m, 1H), 2.56-2.51 (m, 2H), 2.47-2.42 (m, 1H), 2.23-2.09 (m, 1H), 2.07-1.94 (m, 1H), 1.77-1.67 (m, 2H), 1.67-1.56 (m, 3H), 1.22-1.09 (m, 2H).

### Example 111. Synthesis of (R)—N-(2-chlorophenyl)-4-((2-((4-((4-(2-(4-(4-((2,6-dioxopiperidin-3-yl)amino)-3-fluorophenyl)piperazin-1-yl)ethyl)piperidin-1-yl)carbamoyl)phenyl)amino)-5-fluoropyrimidin-4-yl)amino)benzamide (Compound 111)

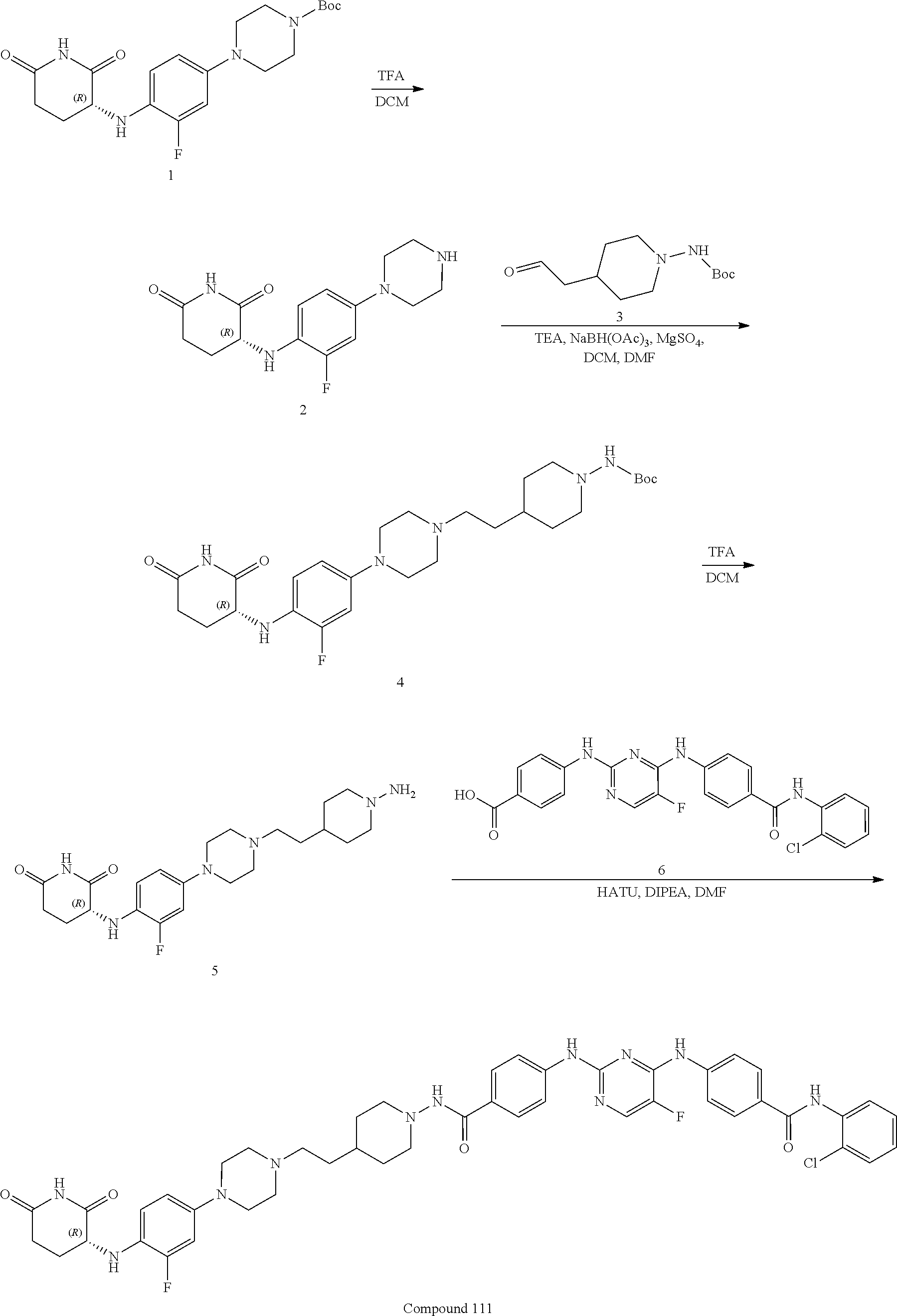

### Step 1. Synthesis of (R)-3-((2-fluoro-4-(piperazin-1-yl)phenyl)amino)piperidine-2,6-dione (2)

To a solution of tert-butyl (R)-4-(4-((2,6-dioxopiperidin-3-yl)amino)-3-fluorophenyl)piperazine-1-carboxylate (200.00 mg, 492.07 μmol) in DCM (2 mL) was added TFA (921.00 mg, 8.08 mmol, 0.6 mL) at 0° C., the mixture was stirred at 0° C. for 0.5 h. LCMS showed a main peak with desired mass. The mixture was concentrated in vacuum to afford (R)-3-((2-fluoro-4-(piperazin-1-yl)phenyl)amino)piperidine-2,6-dione (200 mg, crude, TFA) was obtained as a yellow oil. MS(M+H)⁺=307.1.

### Step 2. Synthesis of tert-butyl (R)-(4-(2-(4-(4-((2,6-dioxopiperidin-3-yl)amino)-3-fluorophenyl)piperazin-1-yl)ethyl) piperidin-1-yl)carbamate (4)

To a solution of (R)-3-((2-fluoro-4-(piperazin-1-yl)phenyl)amino)piperidine-2,6-dione (200 mg, 475.79 mol, TFA) in DCM (3 mL) were added TEA (144.43 mg, 1.43 mmol, 198.67 μL) and MgSO₄ (114.54 mg, 951.57 μmol) and then followed by a solution of tert-butyl (4-(2-oxoethyl)piperidin-1-yl)carbamate (149.88 mg, 618.52 μmol) in DMF (3 mL) and the mixture was stirred at 0° C. for 30 min. NaBH(OAc)₃ (302.51 mg, 1.43 mmol) was added and the resulting mixture was stirred at 0° C. for 1 h. LCMS showed a main peak with desired mass. The mixture was diluted with water (20 mL) extracted with EtOAc (20 mL×3). The combined organic layers were washed with brine (10 mL×2), dried over Na₂SO₄, filtered and the filtrate was concentrated in vacuum. The residue was purified by flash silica gel chromatography (Biotage; 10 g SepaFlash® Silica Flash Column, Eluent of 50-100% EtOAc/Petroleum ether to 10% MeOH/EtOAc gradient @ 80 mL/min) to afford tert-butyl (R)-(4-(2-(4-(4-((2,6-dioxopiperidin-3-yl)amino)-3-fluorophenyl)piperazin-1-yl)ethyl) piperidin-1-yl)carbamate (200 mg, 375.48 μmol, 78.92% yield) as a yellow oil. MS(M+H)⁺=533.3.

### Step 3. Synthesis of (R)-3-((4-(4-(2-(1-aminopiperidin-4-yl)ethyl)piperazin-1-yl)-2-fluorophenyl) amino)piperidine-2,6-dione (5)

To a solution of tert-butyl (R)-(4-(2-(4-(4-((2,6-dioxopiperidin-3-yl)amino)-3-fluorophenyl)piperazin-1-yl)ethyl) piperidin-1-yl)carbamate (200 mg, 375.48 μmol) in DCM (2 mL) was added TFA (921.00 mg, 8.08 mmol, 0.6 mL), the mixture was stirred at 25° C. for 1 h. LCMS showed a peak with desired mass. The mixture was concentrated in vacuum to afford (R)-3-((4-(4-(2-(1-aminopiperidin-4-yl)ethyl)piperazin-1-yl)-2-fluorophenyl)amino)piperidine-2,6-dione (200 mg, crude, TFA) as a yellow oil. MS(M+H)⁺=433.2.

### Step 4. Synthesis of (R)—N-(2-chlorophenyl)-4-((2-((4-((4-(2-(4-(4-((2,6-dioxopiperidin-3-yl)amino)-3-fluorophenyl)piperazin-1-yl)ethyl)piperidin-1-yl)carbamoyl)phenyl)amino)-5-fluoropyrimidin-4-yl)amino)benzamide (Compound 111)

To a solution of 4-((4-((4-((2-chlorophenyl)carbamoyl)phenyl)amino)-5-fluoropyrimidin-2-yl) amino)benzoic acid (174.87 mg, 365.93 μmol) in DMF (2 mL) were added HATU (166.96 mg, 439.11 μmol) and DIPEA (141.88 mg, 1.10 mmol, 191.21 μL). After stirring at 25° C. for 0.5 h, (R)-3-((4-(4-(2-(1-aminopiperidin-4-yl)ethyl)piperazin-1-yl)-2-fluorophenyl)amino)piperidine-2,6-dione (200 mg, 365.93 μmol, TFA) was added and the resulting mixture was stirred at 25° C. for 1 h. LCMS showed a peak (69%) with desired mass. The reaction mixture was poured into ice water (10 mL) and filtered. The filter cake was collected and dried to afford the crude product. The crude product was purified by prep-HPLC (column: Phenomenex luna C18 150*25 mm*10 um; mobile phase: [water (TFA)-ACN]; gradient: 23%-53% B over 10 min) and lyophilized to afford (R)—N-(2-chlorophenyl)-4-((2-((4-((4-(2-(4-(4-((2,6-dioxopiperidin-3-yl)amino)-3-fluorophenyl)piperazin-1-yl)ethyl)piperidin-1-yl)carbamoyl)phenyl)amino)-5-fluoropyrimidin-4-yl)amino)benzamide (152.2 mg, 120.83 μmol, 33.02% yield, 98% purity, 3 TFA) as a white solid. MS(M+H)⁺=892.1.

¹H NMR (400 MHz, DMSO-d₆) δ=10.80 (s, 1H), 9.94 (s, 1H), 9.82-9.61 (m, 3H), 9.54-9.43 (m, 1H), 8.26 (d, J=3.4 Hz, 1H), 8.06-7.99 (m, 4H), 7.83-7.77 (m, 2H), 7.75-7.69 (m, 2H), 7.68-7.62 (m, 1H), 7.60-7.54 (m, 1H), 7.43-7.37 (m, 1H), 7.34-7.27 (m, 1H), 6.91-6.83 (m, 1H), 6.82-6.75 (m, 1H), 6.69-6.63 (m, 1H), 4.33-4.29 (m, 1H), 3.67-3.55 (m, 4H), 3.24-3.08 (m, 6H), 2.93-2.80 (m, 4H), 2.78-2.69 (m, 1H), 2.61-2.54 (m, 1H), 2.13-1.94 (m, 2H), 1.81-1.58 (m, 4H), 1.50-1.30 (m, 3H).

### Example 112. Synthesis of N-[4-[2-[4-[4-(2,6-dioxo-3-piperidyl)-2,5-difluoro-phenyl]piperazin-1-yl]ethyl]-1-piperidyl]-2-fluoro-4-[[5-fluoro-4-[4-[(2-fluorobenzoyl)amino]anilino]pyrimidin-2-yl]amino]benzamide (Compound 112)

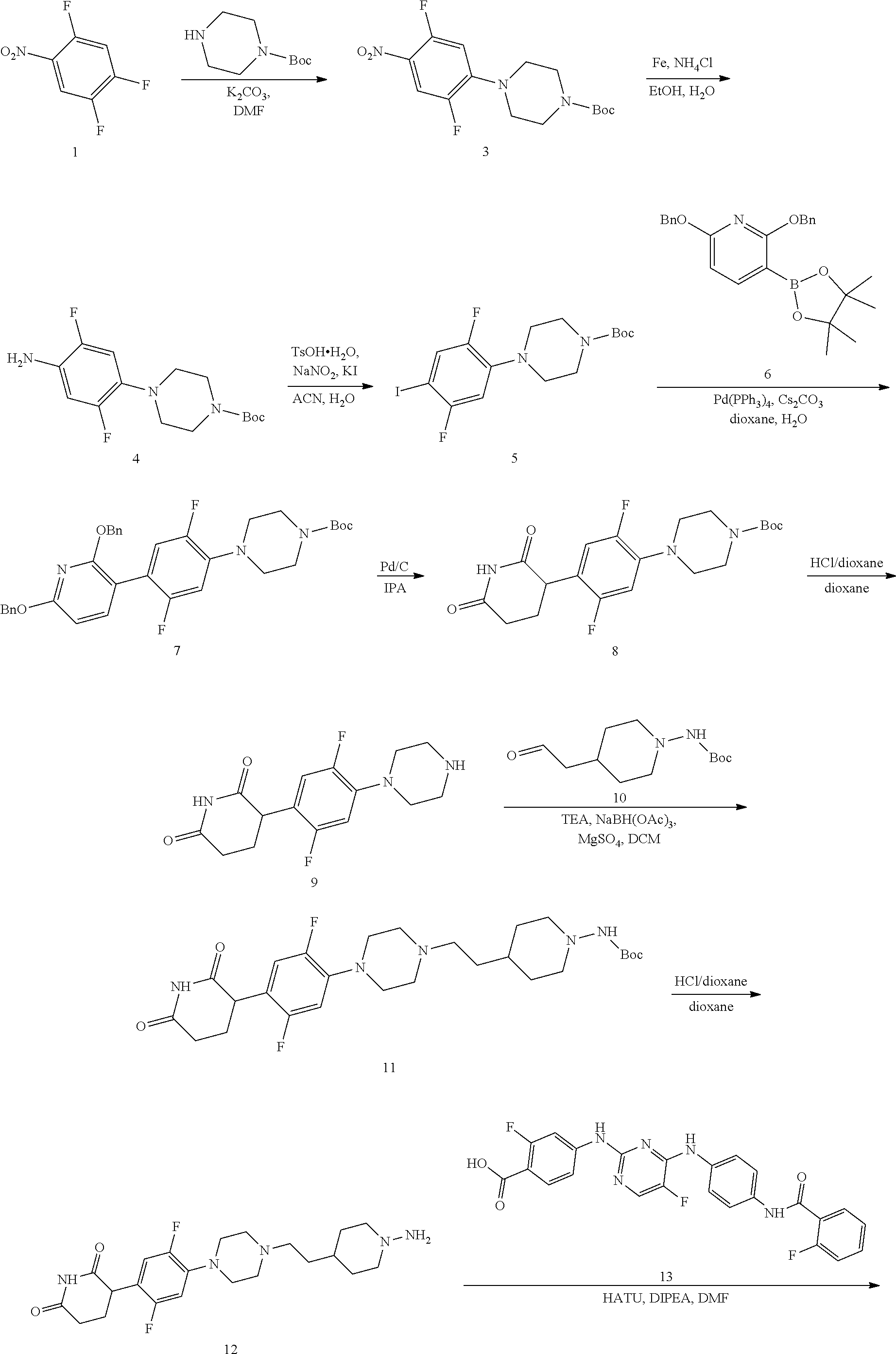

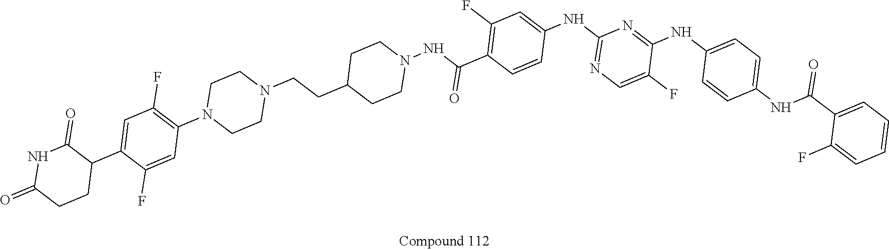 [SMILES: O=C1CCC(c2cc(F)c(N3CCN(CCC4CCN(NC(=O)c5ccc(Nc6ncc(F)c(Nc7ccc(NC(=O)c8ccccc8F)cc7)n6)cc5F)CC4)CC3)cc2F)C(=O)N1]

### Step 1. Synthesis of tert-butyl 4-(2,5-difluoro-4-nitro-phenyl)piperazine-1-carboxylate (3)

To a solution of 1,2,4-trifluoro-5-nitro-benzene (5 g, 28.24 mmol, 3.24 mL) in DMF (50 mL) was added K₂CO₃ (7.80 g, 56.47 mmol) and tert-butyl piperazine-1-carboxylate (5.26 g, 28.24 mmol). The mixture was stirred at 20° C. for 16 hr. LCMS showed a peak (86%) with desired mass. The reaction mixture was diluted with 200 mL of water and filtered. The filter cake was collected and dried to afford tert-butyl 4-(2,5-difluoro-4-nitro-phenyl)piperazine-1-carboxylate (9.5 g, 27.12 mmol, 96.04% yield, 98% purity) as yellow oil. MS(M−55+H)+=288.0.

### Step 2. Synthesis of tert-butyl 4-(4-amino-2,5-difluoro-phenyl)piperazine-1-carboxylate (4)

To a solution of tert-butyl 4-(2, 5-difluoro-4-nitro-phenyl)piperazine-1-carboxylate (9.5 g, 27.67 mmol) and NH₄Cl (7.40 g, 138.35 mmol) in EtOH (80 mL) and H₂O (20 mL) was added Fe (4.64 g, 83.01 mmol) portion-wise. The mixture was stirred at 80° C. for 2 hr. LCMS showed a peak (65%) with desired mass. The reaction mixture was filtrated through celite and concentrated under reduced pressure to remove organic phase. The residue was diluted with 80 mL of aqueous NaHCO₃ solution and extracted with EtOAc (100 mL×3). The combined organic phase was concentrated under reduced pressure. The residue was purified by flash silica gel chromatography (20 g SepaFlash® Silica Flash Column, Eluent of 0~20% EtOAc/Petroleum ether, gradient @ 60 mL/min) to afford tert-butyl 4-(4-amino-2,5-difluoro-phenyl)piperazine-1-carboxylate (7.9 g, 17.90 mmol, 64.69% yield, 71% purity) as brown black solid. MS(M+H)⁺=314.1.

### Step 3. Synthesis of tert-butyl 4-(2,5-difluoro-4-iodo-phenyl)piperazine-1-carboxylate (5)

To a solution of tert-butyl 4-(4-amino-2,5-difluoro-phenyl)piperazine-1-carboxylate (3 g, 9.57 mmol) in ACN (30 mL) and H₂O (30 mL) was added TsOH·H₂O (5.46 g, 28.72 mmol). Then to the mixture was added NaNO₂ (1.32 g, 19.15 mmol) in H₂O (30 mL) at 0° C. After stirring at 0° C. for 1 hr, a solution of KI (3.50 g, 21.06 mmol) in H₂O (30 mL) was added drop-wise. The resulting mixture was stirred at 20° C. for 16 hr. LCMS showed starting material was consumed. The reaction mixture was extracted with EtOAc (80 mL×3) and the combined organic phase was concentrated under reduced pressure to afford a residue. The residue was purified by flash silica gel chromatography (20 g SepaFlash® Silica Flash Column, Eluent of 0-10% EtOAc/Petroleum ether, gradient @ 60 mL/min) to afford tert-butyl 4-(2,5-difluoro-4-iodo-phenyl)piperazine-1-carboxylate (2.29 g, 4.05 mmol, 42.29% yield, 75% purity) as yellow solid. MS(M+H)⁺=425.0.

### Step 4. Synthesis of tert-butyl 4-[4-(2,6-dibenzyloxy-3-pyridyl)-2,5-difluoro-phenyl]piperazine-1-carboxylate (7)

A mixture of tert-butyl 4-(2,5-difluoro-4-iodo-phenyl)piperazine-1-carboxylate (2.29 g, 5.40 mmol), 2,6-dibenzyloxy-3-(4,4,5,5-tetramethyl-1,3,2-dioxaborolan-2-yl)pyridine (2.25 g, 5.40 mmol), Cs₂CO₃ (5.28 g, 16.19 mmol) and Pd(PPh₃)₄ (623.78 mg, 539.81 μmol) in dioxane (20 mL) and H₂O (5 mL) was degassed and purged with N₂ for 3 times, and then the mixture was stirred at 100° C. for 16 hr under N₂ atmosphere. LCMS showed a peak (29%) with desired mass. The reaction mixture was diluted with 50 mL of water and extracted with EtOAc (50 mL×3). The combined organic phase was concentrated under reduced pressure. The residue was purified by flash silica gel chromatography (20 g SepaFlash® Silica Flash Column, Eluent of 0-5% EtOAc/Petroleum ether, gradient @ 60 mL/min) to afford tert-butyl 4-[4-(2,6-dibenzyloxy-3-pyridyl)-2,5-difluoro-phenyl]piperazine-1-carboxylate (2.55 g, 4.12 mmol, 76.37% yield, 95% purity) as yellow oil. MS(M+H)⁺=588.2.

¹H NMR (400 MHz, CDCl₃) δ=7.53 (dd, J=1.1, 8.1 Hz, 1H), 7.46-7.27 (m, 10H), 7.12 (dd, J=6.9, 13.0 Hz, 1H), 6.77-6.64 (m, 1H), 6.47 (d, J=8.1 Hz, 1H), 5.40-5.35 (m, 4H), 3.67-3.56 (m, 4H), 3.13-2.99 (m, 4H), 1.50 (s, 9H).

### Step 5. Synthesis of tert-butyl 4-[4-(2,6-dioxo-3-piperidyl)-2,5-difluoro-phenyl]piperazine-1-carboxylate (8)

(Note: the reaction was conducted by flow chemistry platform) To a solution of tert-butyl 4-[4-(2,6-dibenzyloxy-3-pyridyl)-2,5-difluoro-phenyl]piperazine-1-carboxylate (2.55 g, 4.34 mmol) in IPA (260 g) was stirred at 20° C. until becoming clear solution. Adjust the H₂ back pressure regulator to 1 MPa, the flow rate of H₂ to 30 mL/min, heated the fixed bed (5% Pd/C, 15 g) to 50° C. Then the solution was pumped into the reactor at a flow rate of 0.33 mL/min. After the reaction was finished, wash the tubing with IPA 300 mL, collected all the reaction solution for analysis. LCMS showed a peak (98%) with desired mass. The fraction was concentrated under reduced pressure to afford tert-butyl 4-[4-(2,6-dioxo-3-piperidyl)-2,5-difluoro-phenyl]piperazine-1-carboxylate (370 mg, 903.70 μmol, 100.00% yield) as white solid. MS(M−55+H)+=354.0.

¹H NMR (400 MHz, CDCl₃-d) δ=7.99 (br s, 1H), 6.86 (dd, J=6.8, 12.4 Hz, 1H), 6.67 (dd, J=7.2, 11.6 Hz, 1H), 3.83 (dd, J=5.6, 11.2 Hz, 1H), 3.65-3.55 (m, 4H), 3.09-2.97 (m, 4H), 2.87-2.77 (m, 1H), 2.76-2.62 (m, 1H), 2.33-2.15 (m, 2H), 1.49 (s, 9H).

### Step 6. Synthesis of 3-(2,5-difluoro-4-piperazin-1-yl-phenyl)piperidine-2,6-dione (9)

To a solution of tert-butyl 4-[4-(2,6-dioxo-3-piperidyl)-2,5-difluoro-phenyl]piperazine-1-carboxylate (370 mg, 903.70 μmol) in dioxane (1 mL) was added HCl/dioxane (2 M, 4 mL). The mixture was stirred at 20° C. for 2 hr. LCMS showed starting material was consumed and desired mass was detected. The reaction mixture was concentrated under reduced pressure to afford 3-(2, 5-difluoro-4-piperazin-1-yl-phenyl)piperidine-2, 6-dione (300 mg, crude, HCl) as gray solid. MS (M+H)⁺=310.1.

### Step 7. Synthesis of tert-butyl N-[4-[2-[4-[4-(2,6-dioxo-3-piperidyl)-2,5-difluoro-phenyl]piperazin-1-yl]ethyl]-1-piperidyl]carbamate (11)

To a solution of 3-(2,5-difluoro-4-piperazin-1-yl-phenyl)piperidine-2,6-dione (300 mg, 867.63 μmol, HCl) and tert-butyl N-[4-(2-oxoethyl)-1-piperidyl]carbamate (315.36 mg, 1.30 mmol) in DCM (10 mL) was added MgSO₄ (104.43 mg, 867.63 mol) and TEA (263.38 mg, 2.60 mmol, 362.29 μL). The mixture was stirred at 20° C. for 0.5 hr. To the mixture was added NaBH(OAc)₃ (551.66 mg, 2.60 mmol). The resulting mixture was stirred at 20° C. for 16 hr. LCMS showed a peak (85%) with desired mass. The mixture was diluted with 20 mL of water and extracted with DCM (20 mL×2). The combined organic phase was concentrated under reduced pressure. The residue was triturated with 20 mL of MTBE to afford tert-butyl N-[4-[2-[4-[4-(2,6-dioxo-3-piperidyl)-2,5-difluoro-phenyl]piperazin-1-yl]ethyl]-1-piperid yl]carbamate (260 mg, 485.41 μmol, 55.95% yield, 100% purity) as gray solid. MS(M+H)⁺=536.3.

### Step 8. Synthesis of 3-[4-[4-[2-(1-amino-4-piperidyl)ethyl]piperazin-1-yl]-2,5-difluoro-phenyl] piperidine-2,6-dione (12)

To a solution of tert-butyl N-[4-[2-[4-[4-(2,6-dioxo-3-piperidyl)-2,5-difluoro-phenyl]piperazin-1-yl]ethyl]-1-piperid yl]carbamate (150 mg, 280.05 μmol) in dioxane (2 mL) was added HCl/dioxane (2 M, 4 mL). The mixture was stirred at 20° C. for 2 hr. LCMS showed starting material was consumed and desired mass was detected. The reaction mixture was concentrated under reduced pressure to afford 3-[4-[4-[2-(1-amino-4-piperidyl)ethyl]piperazin-1-yl]-2,5-difluoro-phenyl]piperidine-2,6-dione (130 mg, crude, HCl) as gray solid. MS(M+H)⁺=436.2.

### Step 9. Synthesis of N-[4-[2-[4-[4-(2,6-dioxo-3-piperidyl)-2,5-difluoro-phenyl]piperazin-1-yl]ethyl]-1-piperidyl]-2-fluoro-4-[[5-fluoro-4-[4-[(2-fluorobenzoyl)amino]anilino]pyrimidin-2-yl]amino]benzamide (Compound 112)

To a solution of 2-fluoro-4-[[5-fluoro-4-[4-[(2-fluorobenzoyl)amino]anilino]pyrimidin-2-yl]amino]benzoic acid (132.05 mg, 275.44 μmol) in DMF (3 mL) was added HATU (125.68 mg, 330.53 μmol), DIPEA (106.80 mg, 826.32 μmol) and 3-[4-[4-[2-(1-amino-4-piperidyl)ethyl]piperazin-1-yl]-2,5-difluoro-phenyl]piperidine-2,6-dione (130 mg, 275.44 μmol, HCl). The mixture was stirred at 20° C. for 2 hr. LCMS showed a peak (48%) with desired mass. The reaction mixture was diluted with 10 mL of aqueous NaHCO₃ solution and filtered. The filter cake was purified by prep-HPLC (column: Phenomenex luna C18 150*40 mm*15 um; mobile phase: [water (TFA)-ACN]; gradient: 20%-50% B over 10 min) and lyophilized to afford N-[4-[2-[4-[4-(2, 6-dioxo-3-piperidyl)-2,5-difluoro-phenyl]piperazin-1-yl]ethyl]-1-piperidyl]-2-fluoro-4-[[5-fluoro-4-[4-[(2-fluorobenzoyl)amino]anilino]pyrimidin-2-yl]amino]benzamide (58.3 mg, 64.35 μmol, 23.36% yield, 99% purity) as white solid. MS(M+H)⁺=897.3.

¹H NMR (400 MHz, DMSO-d₆) δ=10.88 (s, 1H), 10.43 (s, 1H), 9.80-9.48 (m, 3H), 9.34-9.12 (m, 1H), 8.18 (d, J=3.5 Hz, 1H), 7.81 (br d, J=14.1 Hz, 1H), 7.76-7.72 (m, 4H), 7.68 (br t, J=6.9 Hz, 1H), 7.64-7.54 (m, 1H), 7.44 (br d, J=3.9 Hz, 2H), 7.39-7.30 (m, 2H), 7.22 (br dd, J=6.9, 13.1 Hz, 1H), 7.02 (br dd, J=7.4, 11.7 Hz, 1H), 4.02-3.98 (m, 1H), 3.66-3.49 (m, 4H), 3.30-3.14 (m, 4H), 3.12-2.99 (m, 4H), 2.82-2.62 (m, 3H), 2.57-2.53 (m, 1H), 2.29-2.13 (m, 1H), 2.04-1.89 (m, 1H), 1.80-1.52 (m, 4H), 1.35-1.30 (m, 2H).

### Example 113. Synthesis of N-(2-chlorophenyl)-4-((2-((4-((4-(2-((2S)-4-(4-(2,6-dioxopiperidin-3-yl)phenyl)-2-methylpiperazin-1-yl)ethyl)piperidin-1-yl)carbamoyl)phenyl)amino)-5-fluoropyrimidin-4-yl)amino)benzamide (Compound 113)

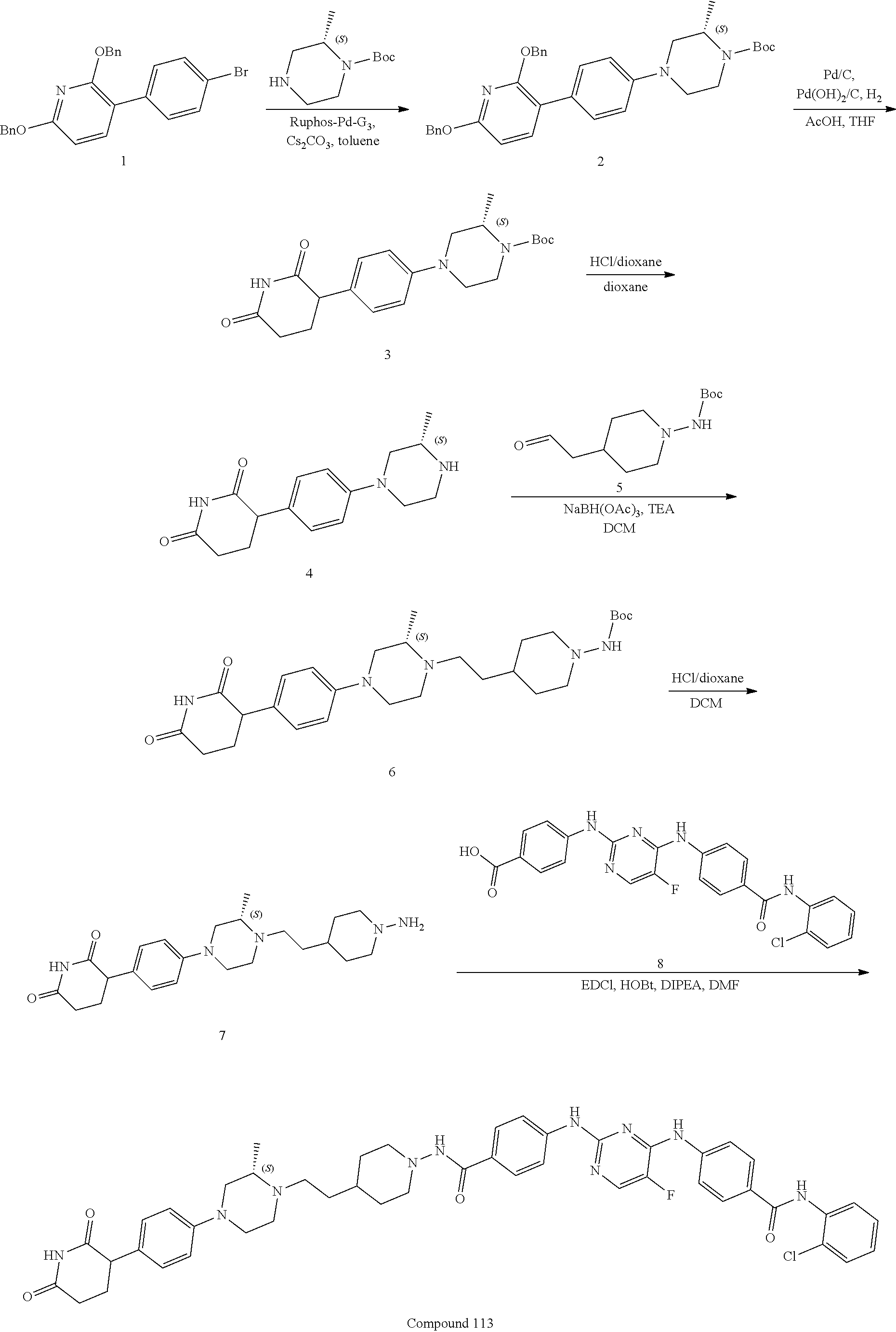

### Step 1. Synthesis of tert-butyl (S)-4-(4-(2,6-bis(benzyloxy)pyridin-3-yl)phenyl)-2-methylpiperazine-1-carboxylate (2)

To a solution of tert-butyl (S)-2-methylpiperazine-1-carboxylate (217.63 mg, 1.09 mmol), 2,6-bis(benzyloxy)-3-(4-bromophenyl)pyridine (500 mg, 1.09 mmol) in toluene (10 mL) was added Cs₂CO₃ (1.06 g, 3.26 mmol) and RuPhos-Pd-G₃ (90.88 mg, 108.66 μmol) under N₂ atmosphere. The suspension was degassed and purged with N₂ for 3 times. The mixture was stirred under N₂ atmosphere at 100° C. for 16 hr. LCMS showed ~59% desired mass was detected. The reaction mixture was allowed to cool down to room temperature and filtered directly. The filter cake was washed with EtOAc (50 mL). The combined filtrate was poured into water (40 mL) and extracted with EtOAc (30 mL×2). The combined organic phases was dried over anhydrous Na₂ SO₄. After filtration, the filtrate was concentrated under reduced pressure. The residue was purified by flash silica gel chromatography (Biotage, 12 g silica gel column, eluted with 10% EtOAc in petroleum ether) to afford tert-butyl (S)-4-(4-(2, 6-bis(benzyloxy)pyridin-3-yl)phenyl)-2-methylpiperazine-1-carboxylate (510 mg, 883.51 μmol, 81.31% yield, 98% purity) as a light yellow oil. MS(M+H)⁺=566.3.

¹H NMR (400 MHz, DMSO-d₆) δ=7.67 (d, J=8.1 Hz, 1H), 7.29-7.44 (m, 12H), 6.93 (d, J=8.9 Hz, 2H), 6.51 (d, J=8.1 Hz, 1H), 5.39 (s, 2H), 5.35 (s, 2H), 4.16-4.25 (m, 1H), 3.76-3.83 (m, 1H), 3.60 (br d, J=11.9 Hz, 1H), 3.51 (br d, J=12.3 Hz, 1H), 3.11-3.20 (m, 1H), 2.82 (dd, J=12.3, 3.8 Hz, 1H), 2.62 (td, J=11.9, 3.6 Hz, 1H), 1.42 (s, 9H), 1.15-1.18 (m, 3H).

### Step 2. Synthesis of tert-butyl (2S)-4-(4-(2,6-dioxopiperidin-3-yl)phenyl)-2-methylpiperazine-1-carboxylate (3)

To a mixture of Pd/C (94.02 mg, 88.35 μmol, 10% purity) and Pd(OH)₂/C (60 mg, 88.35 μmol, 20% purity) in THF (15 mL) was added AcOH (53.06 mg, 883.51 μmol) and tert-butyl (S)-4-(4-(2,6-bis (benzyloxy)pyridin-3-yl)phenyl)-2-methylpiperazine-1-carboxylate (510 mg, 883.51 mol) under Ar atmosphere. The resulting suspension was degassed and purged with H₂ for 3 times. The mixture was stirred under H₂ (50 Psi) at 30° C. for 48 hr. LCMS showed one main peak with desired mass was detected. The reaction mixture was filtered and washed with EtOAc (3×30 mL). The combined organic phase was concentrated under reduced pressure to afford tert-butyl (2S)-4-(4-(2,6-dioxopiperidin-3-yl)phenyl)-2-methylpiperazine-1-carboxylate (300 mg, 696.83 μmol, 78.87% yield, 90% purity) as a yellow oil. MS(M+H)⁺=388.4.

### Step 3. Synthesis of 3-(4-((S)-3-methylpiperazin-1-yl)phenyl)piperidine-2,6-dione (4)

To a solution of tert-butyl rac-(2S)-4-[4-(2,6-dioxo-3-piperidyl)phenyl]-2-methyl-piperazine-1-carboxylate (300 mg, 696.83 μmol) in dioxane (0.5 mL) was added HCl/dioxane (2 M, 13.50 mL) at 20° C. The mixture was stirred at 20° C. for 2 hr. LCMS showed one main peak with desired mass. The mixture was concentrated under reduced pressure to afford 3-(4-((S)-3-methylpiperazin-1-yl)phenyl)piperidine-2,6-dione (330 mg, crude, HCl) as a yellow solid. MS (M+H)⁺=288.1.

### Step 4. Synthesis of tert-butyl (4-(2-((2S)-4-(4-(2,6-dioxopiperidin-3-yl)phenyl)-2-methylpiperazin-1-yl)ethyl)piperidin-1-yl)carbamate (6)

To a solution of 3-(4-((S)-3-methylpiperazin-1-yl)phenyl)piperidine-2, 6-dione (330 mg, 1.02 mmol, HCl) in DCM (10 mL) was added TEA (515.61 mg, 5.10 mmol) and tert-butyl (4-(2-oxoethyl)piperidin-1-yl) carbamate (297 mg, 1.23 mmol) at 20° C. The mixture was stirred at 20° C. for 30 min, followed by adding NaBH(OAc)₃ (647.96 mg, 3.06 mmol) in portions. The mixture was stirred at 20° C. for 16 hr. LCMS showed one main peak with desired mass was detected. The mixture was poured into water (30 mL) and then extracted with DCM (30 mL×3). The combined organic phases were dried over anhydrous Na₂SO₄, filtered and concentrated under reduced pressure to afford tert-butyl (4-(2-((2S)-4-(4-(2, 6-dioxopiperidin-3-yl)phenyl)-2-methylpiperazin-1-yl)ethyl)piperidin-1-yl) carbamate (280 mg, 496.04 μmol, 48.67% yield, 91% purity) as a yellow oil. MS(M+H)⁺=514.3.

### Step 5. Synthesis of 3-(4-((S)-4-(2-(1-aminopiperidin-4-yl)ethyl)-3-methylpiperazin-1-yl)phenyl)piperidine-2,6-dione (7)

To a solution of tert-butyl (4-(2-((2S)-4-(4-(2,6-dioxopiperidin-3-yl)phenyl)-2-methylpiperazin-1-yl)ethyl)piperidin-1-yl) carbamate (240 mg, 425.18 μmol) in DCM (1 mL) was added HCl/dioxane (2 M, 20 mL). The mixture was stirred at 15° C. for 2 hr. LCMS showed ~63% peak with desired mass. The mixture was concentrated under reduced pressure to afford 3-(4-((S)-4-(2-(1-aminopiperidin-4-yl)ethyl)-3-methylpiperazin-1-yl)phenyl)piperidine-2,6-dione (270 mg, crude, 2HCl) as a yellow solid. MS(M+H)⁺=414.3.

### Step 6. Synthesis of N-(2-chlorophenyl)-4-((2-((4-((4-(2-((2S)-4-(4-(2,6-dioxopiperidin-3-yl)phenyl)-2-methylpiperazin-1-yl)ethyl)piperidin-1-yl)carbamoyl)phenyl)amino)-5-fluoropyrimidin-4-yl) amino)benzamide (Compound 113)

To a solution of 3-(4-((S)-4-(2-(1-aminopiperidin-4-yl)ethyl)-3-methylpiperazin-1-yl)phenyl)piperidine-2,6-dione (100 mg, 205.56 μmol, 2HCl), 4-((4-((4-((2-chlorophenyl)carbamoyl)phenyl)amino)-5-fluoropyrimidin-2-yl)amino) benzoic acid (78.59 mg, 164.45 μmol) in DMF (8 mL) was added DIPEA (132.84 mg, 1.03 mmol, 179.02 μL), EDCI (78.81 mg, 411.12 μmol) and HOBt (55.55 mg, 411.12 mol). After stirring at 15° C. for 16 hr, 3-(4-((S)-4-(2-(1-aminopiperidin-4-yl)ethyl)-3-methylpiperazin-1-yl)phenyl)piperidine-2,6-dione (50 mg) and DIPEA (0.1 mL) were added and the resulting mixture was stirred at 15° C. for 16 hr. LCMS showed ~21% peak with desired mass. The mixture was diluted with water (15 mL) and extracted with EtOAc (15 mL). The organic phase washed with water three times (15 mL×3) and washed again with saturated NaHCO₃ aqueous solution (15 mL). The organic phase was dried over anhydrous Na₂SO₄, filtered and concentrated under reduced pressure. The residue was purified by flash silica gel chromatography (Biotage, 4 g silica gel column, eluted with 47% MeOH in EtOAc) to give the crude product (50 mg). The residue (50 mg) was purified by prep-HPLC (neutral condition; column: Waters Xbridge 150*25 mm*5 um; mobile phase: [water (NH₄HCO₃)-ACN]; gradient: 38%-68% B over 9 min) and lyophilized to afford N-(2-chlorophenyl)-4-((2-((4-((4-(2-((2S)-4-(4-(2,6-dioxopiperidin-3-yl)phenyl)-2-methylpiperazin-1-yl)ethyl)piperidin-1- yl)carbamoyl)phenyl)amino)-5-fluoropyrimidin-4-yl) amino)benzamide (5.4 mg, 5.90 μmol, 15.17% yield, 95.5% purity) as a white solid. MS(M+H)⁺=873.5.

¹H NMR (400 MHz, DMSO-d₆) δ=10.77 (s, 1H), 9.93 (s, 1H), 9.74 (s, 1H), 9.60 (s, 1H), 9.12 (s, 1H), 8.24 (d, J=3.2 Hz, 1H), 7.99-8.04 (m, 4H), 7.74-7.78 (m, 2H), 7.68-7.71 (m, 2H), 7.65 (dd, J=8.0, 1.6 Hz, 1H), 7.56 (dd, J=7.6, 1.2 Hz, 1H), 7.39 (td, J=7.8, 1.4 Hz, 1H), 7.26-7.32 (m, 1H), 7.04 (d, J=8.6 Hz, 2H), 6.88 (d, J=8.8 Hz, 2H), 3.72 (dd, J=11.2, 4.8 Hz, 1H), 2.97 (br d, J=10.4 Hz, 2H), 2.86-2.91 (m, 1H), 2.65-2.80 (m, 4H), 2.07-2.35 (m, 5H), 1.95-2.05 (m, 2H), 1.72-1.68 (m, 2H), 1.36-1.43 (m, 2H), 1.23-1.32 (m, 6H), 1.06 (br d, J=5.2 Hz, 3H).

### Example 114. Synthesis of N-(2-chlorophenyl)-4-((2-((4-((4-(2-((3S)-4-(4-(2,6-dioxopiperidin-3-yl)phenyl)-3-methylpiperazin-1-yl)ethyl)piperidin-1-yl)carbamoyl)phenyl)amino)-5-fluoropyrimidin-4-yl)amino)benzamide (Compound 114)

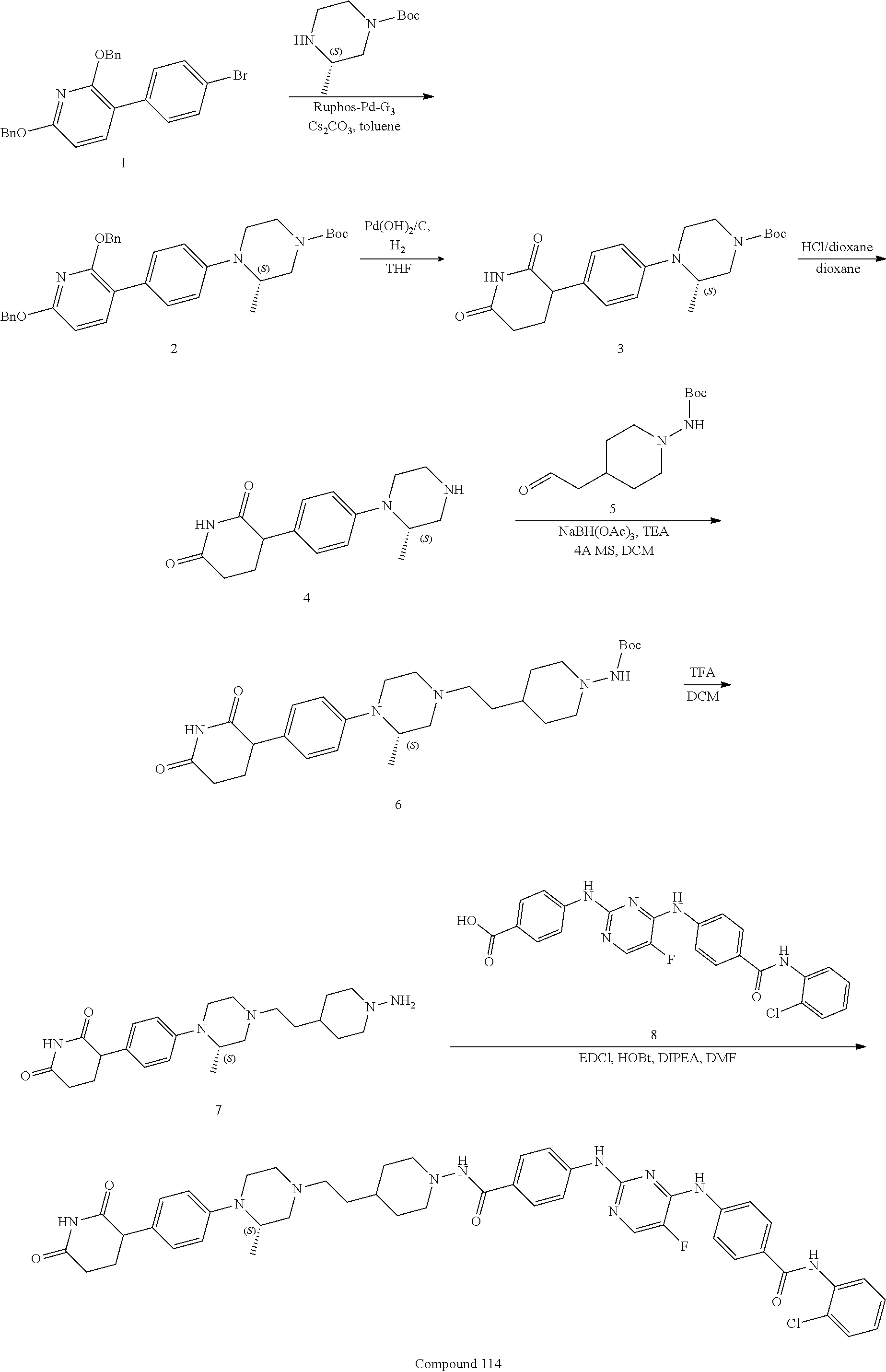

### Step 1. Synthesis of tert-butyl (S)-4-(4-(2,6-bis(benzyloxy)pyridin-3-yl)phenyl)-3-methylpiperazine-1-carboxylate (2)

To a solution of 2,6-dibenzyloxy-3-(4-bromophenyl)pyridine (1 g, 2.02 mmol), tert-butyl rac-(3S)-3-methylpiperazine-1-carboxylate (444.23 mg, 2.22 mmol) in toluene (15 mL) was added RuPhos-Pd-G3 (168.65 mg, 201.64 μmol) and Cs₂CO₃ (1.97 g, 6.05 mmol). The mixture was stirred at 100° C. for 16 hr. LCMS showed the starting material was consumed completely and the desired mass. The mixture was filtered and the filtrate was concentrated. The residue was purified by flash silica gel chromatography (ISCO®; 20 g SepaFlash® Silica Flash Column, Eluent of ~25% EtOAc/Petroleum ether gradient @ 40 mL/min) to afford tert-butyl (S)-4-(4-(2,6-bis(benzyloxy)pyridin-3-yl)phenyl)-3-methylpiperazine-1-carboxylate (600 mg, 1.04 mmol, 51.55% yield, 98% purity) as a yellow oil. MS(M+H)⁺=566.3.

¹H NMR (400 MHz, CDCl₃) δ=7.59 (d, J=8.0 Hz, 1H), 7.49 (d, J=8.8 Hz, 2H), 7.45-7.39 (m, 12H), 7.39-7.34 (m, 4H), 7.33-7.28 (m, 3H), 6.91 (d, J=8.6 Hz, 2H), 6.45 (d, J=8.1 Hz, 1H), 5.43 (s, 2H), 5.35 (s, 2H), 3.93-3.84 (m, 1H), 3.79 (d, J=13.0 Hz, 1H), 3.36 (dd, J=3.1, 12.9 Hz, 1H), 3.29-3.06 (m, 3H), 1.49-1.45 (m, 1H), 1.05 (d, J=6.5 Hz, 3H).

### Step 2. Synthesis of tert-butyl (3S)-4-(4-(2,6-dioxopiperidin-3-yl)phenyl)-3-methylpiperazine-1-carboxylate (3)

To a mixture of Pd/C (100 mg, 93.97 μmol, 10% purity) and Pd(OH)₂/C (100 mg, 142.42 μmol, 20% purity) in THF (10 mL) was added tert-butyl (S)-4-(4-(2,6-bis(benzyloxy)pyridin-3-yl)phenyl)-3-methylpiperazine-1-carboxylate (600 mg, 1.04 mmol) under Ar atmosphere. The suspension was degassed and purged with H₂ for 3 times. The mixture was stirred under H₂ (50 Psi) at 30° C. for 48 hr. LCMS showed a main peak with desired mass. The mixture was filtered and the filtrate was concentrated to afford (3S)-4-(4-(2,6-dioxopiperidin-3-yl)phenyl)-3-methylpiperazine-1-carboxylate (390 mg, 1.01 mmol, 96.84% yield) as a blue solid. MS (M+H)⁺=388.1.

¹H NMR (400 MHz, DMSO-d₆) δ=10.77 (s, 1H), 7.06 (d, J=8.5 Hz, 2H), 6.86 (d, J=8.6 Hz, 2H), 3.96-3.88 (m, 2H), 3.76-3.67 (m, 3H), 3.22 (d, J=11.6 Hz, 2H), 2.94-2.88 (m, 1H), 2.64-2.57 (m, 1H), 2.47-2.38 (m, 1H), 2.19-2.08 (m, 1H), 2.05-1.95 (m, 1H), 1.89 (s, 2H), 1.42 (s, 9H), 0.89 (d, J=6.4 Hz, 3H).

### Step 3. Synthesis of 3-(4-((S)-2-methylpiperazin-1-yl)phenyl)piperidine-2,6-dione (4)

To a solution of tert-butyl (3S)-4-(4-(2,6-dioxopiperidin-3-yl)phenyl)-3-methylpiperazine-1-carboxylate (390 mg, 1.01 mmol) in dioxane (2 mL) was added HCl/dioxane (2 M, 10 mL). The mixture was stirred at 15° C. for 16 hr. LCMS showed the starting material was consumed completely and a main peak with desired mass. The mixture was concentrated to afford 3-[4-[(2S)-2-methylpiperazin-1-yl]phenyl]piperidine-2,6-dione (350 mg, crude, 2HCl) as a yellow solid. MS (M+H)⁺=288.1.

### Step 4. Synthesis of tert-butyl (4-(2-((3S)-4-(4-(2,6-dioxopiperidin-3-yl)phenyl)-3-methylpiperazin-1-yl)ethyl)piperidin-1-yl)carbamate (6)

To a solution of 3-[4-[(2S)-2-methylpiperazin-1-yl]phenyl]piperidine-2,6-dione (290 mg, 804.93 μmol, 2HCl) and tert-butyl N-[4-(2-oxoethyl)-1-piperidyl]carbamate (195.05 mg, 804.93 μmol) in DCM (6 mL) was added TEA (244.35 mg, 2.41 mmol, 336.11 μL) and 4 Å MS (200 mg). After stirring at 25° C. for 0.5 hr, NaBH(OAc)₃ (255.90 mg, 1.21 mmol) was added and the resulting mixture was stirred at 15° C. for 15.5 hr. LCMS showed the starting material was consumed completely and a main peak with desired mass. The reaction mixture was diluted with DCM (30 mL), washed with aq. NaHCO₃ (20 mL), dried over Na₂SO₄. The mixture was filtered and the filtrate was concentrated. The residue was purified by flash silica gel chromatography (ISCO®; 20 g SepaFlash® Silica Flash Column, Eluent of ~8% MeOH/EtOAc ether gradient @ 80 mL/min) to afford tert-butyl N-[4-[2-[(3S)-4-[4-(2,6-dioxo-3-piperidyl)phenyl]-3-methyl-piperazin-1-yl]ethyl]-1-piperidyl]carbamate (270 mg, 473.07 μmol, 58.77% yield, 90% purity) as a yellow solid. MS(M+H)⁺=514.2.

### Step 5. Synthesis of 3-(4-((S)-4-(2-(1-aminopiperidin-4-yl)ethyl)-2-methylpiperazin-1-yl)phenyl)piperidine-2,6-dione (7)

To a solution of tert-butyl N-[4-[2-[(3S)-4-[4-(2,6-dioxo-3-piperidyl)phenyl]-3-methyl-piperazin-1-yl]ethyl]-1-piperidyl]carbamate (240 mg, 420.50 μmol) in DCM (10 mL) was added TFA (2 mL). The mixture was stirred at 20° C. for 16 hr. LCMS showed a main peak with desired mass. The mixture was concentrated to afford 3-[4-[(2S)-4-[2-(1-amino-4-piperidyl)ethyl]-2-methyl-piperazin-1-yl]phenyl]piperidine-2,6-dione (300 mg, crude, 2TFA) as a yellow oil. MS (M+H)⁺=414.2.

### Step 6. Synthesis of N-(2-chlorophenyl)-4-((2-((4-((4-(2-((3S)-4-(4-(2,6-dioxopiperidin-3-yl)phenyl)-3-methylpiperazin-1-yl)ethyl)piperidin-1-yl)carbamoyl)phenyl)amino)-5-fluoropyrimidin-4-yl) amino)benzamide (Compound 114)

To a solution of 4-[[4-[4-[(2-chlorophenyl)carbamoyl]anilino]-5-fluoro-pyrimidin-2-yl]amino]benzoic acid (100 mg, 209.26 μmol), 3-[4-[(2S)-4-[2-(1-amino-4-piperidyl)ethyl]-2-methyl-piperazin-1-yl]phenyl]piperidine-2,6-dione (268.52 mg, 418.52 μmol, 2TFA), HOBt (42.41 mg, 313.89 μmol) in DMF (3 mL) was added DIPEA (135.23 mg, 1.05 mmol, 182.25 μL) and EDCI (60.17 mg, 313.89 μmol). The mixture was stirred at 15° C. for 16 hr. LCMS showed the starting material was consumed completely and a ~50% peak with desired mass. The reaction mixture was diluted with EtOAc (10 mL), washed with aq. NaHCO₃ (5 mL×3), dried over Na₂SO₄ and concentrated. The residue was purified by prep-HPLC (neutral condition; column: Waters Xbridge 150*25 mm*5 um; mobile phase: [water(NH₄HCO₃)-ACN]; gradient: 38%-68% B over 9 min) and lyophilized to afford N-(2-chlorophenyl)-4-[[2-[4-[[4-[2-[(3S)-4-[4-(2,6-dioxo-3-piperidyl)phenyl]-3-methyl-piperazin-1-yl]ethyl]-1-piperidyl]carbamoyl]anilino]-5-fluoro-pyrimidin-4-yl]amino]benzamide (10.6 mg, 11.04 μmol, 5.28% yield, 91% purity) as a white solid. MS(M+H)⁺=873.4.

¹H NMR (400 MHz, DMSO-d₆) δ=10.77 (s, 1H), 9.93 (s, 1H), 9.74 (s, 1H), 9.61 (s, 1H), 9.13 (s, 1H), 8.24 (d, J=3.5 Hz, 1H), 8.10-7.94 (m, 4H), 7.80-7.73 (m, 2H), 7.73-7.67 (m, 2H), 7.65 (d, J=6.9 Hz, 1H), 7.56 (dd, J=1.3, 7.9 Hz, 1H), 7.42-7.36 (m, 1H), 7.32-7.26 (m, 1H), 7.04 (d, J=8.4 Hz, 2H), 6.83 (d, J=8.8 Hz, 2H), 4.01-3.89 (m, 1H), 3.72 (dd, J=4.8, 10.9 Hz, 1H), 3.01-2.91 (m, 3H), 2.85 (d, J=11.1 Hz, 1H), 2.73-2.63 (m, 3H), 2.39-2.22 (m, 4H), 2.15-1.95 (m, 4H), 1.65-1.71 (m, 2H), 1.47-1.38 (m, 2H), 1.33-1.28 (m, 2H), 1.27-1.20 (m, 3H), 1.00 (d, J=6.4 Hz, 3H).

### Example 115. Synthesis of N-(2-chlorophenyl)-4-((2-((4-((4-(2-((2R)-4-(4-(2,6-dioxopiperidin-3-yl)phenyl)-2-methylpiperazin-1-yl)ethyl)piperidin-1-yl)carbamoyl)phenyl)amino)-5-fluoropyrimidin-4-yl)amino)benzamide (Compound 115)

In a manner similar to the other examples obtained the titled compound.

### Example 116. Synthesis of N-(2-chlorophenyl)-4-((2-((4-((4-(2-((3R)-4-(4-(2,6-dioxopiperidin-3-yl)phenyl)-3-methylpiperazin-1-yl)ethyl)piperidin-1-yl)carbamoyl)phenyl)amino)-5-fluoropyrimidin-4-yl)amino)benzamide (Compound 116)

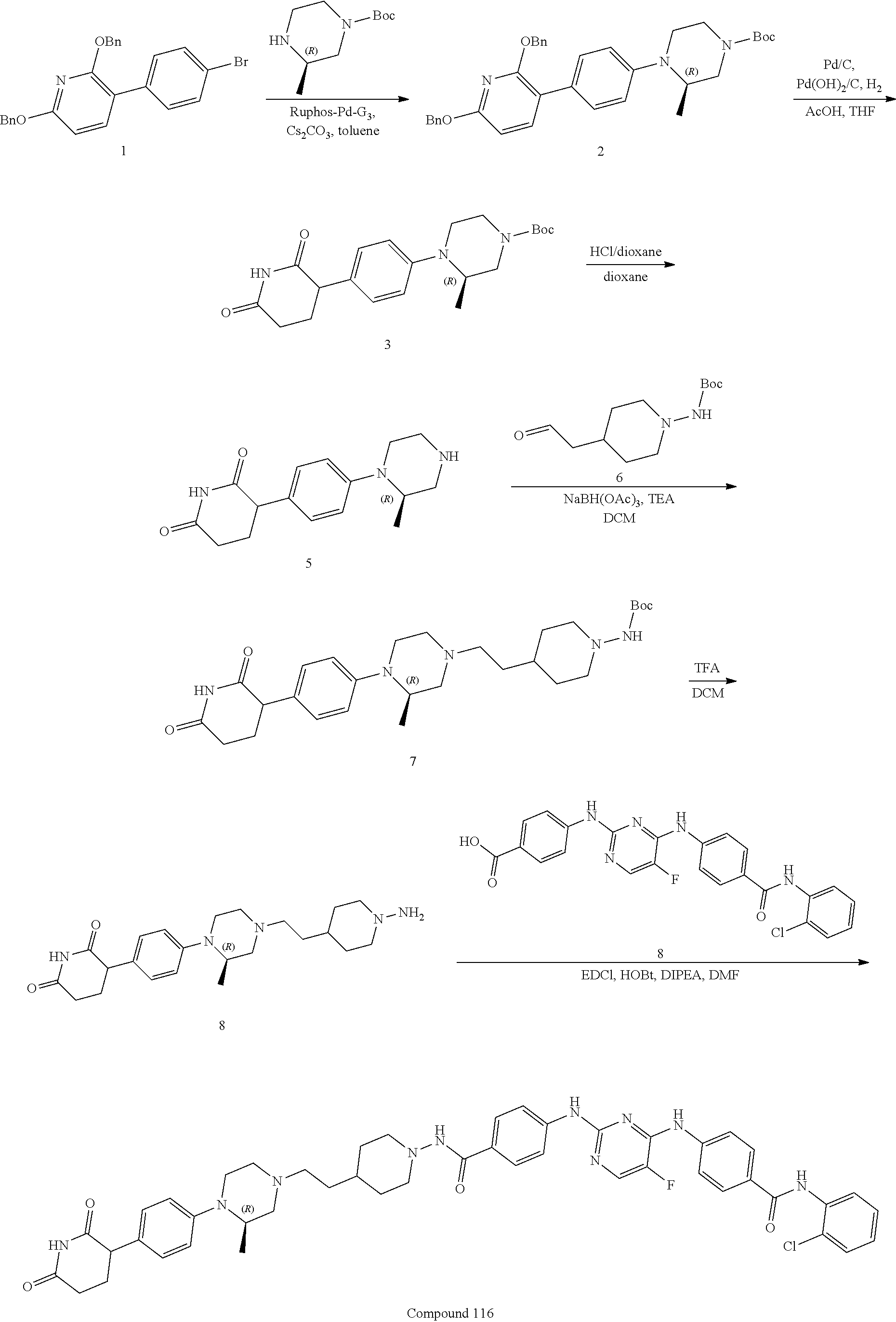

### Step 1. Synthesis of tert-butyl (R)-4-(4-(2,6-bis(benzyloxy)pyridin-3-yl)phenyl)-3-methylpiperazine-1-carboxylate

To a solution of tert-butyl (R)-3-methylpiperazine-1-carboxylate (217.63 mg, 1.09 mmol), 2,6-bis(benzyloxy)-3-(4-bromophenyl)pyridine (500.00 mg, 1.09 mmol) in toluene (15 mL) was added Cs₂CO₃ (1.06 g, 3.26 mmol) and RuPhos-Pd-G3 (90.88 mg, 108.66 μmol) under N₂ atmosphere. The suspension was degassed and purged with N₂ for 3 times. The mixture was stirred under N₂ atmosphere at 100° C. for 16 hr. LCMS showed ~31% desired mass was detected. The reaction mixture was allowed to cool down to room temperature. Then the mixture was poured into water (40 mL) and extracted with EtOAc (30 mL×2), the combined organic phases was dried over anhydrous Na₂SO₄. After filtration, the filtrate was concentrated under reduced pressure to afford a residue. The residue was purified by flash silica gel chromatography (Biotage, 12 g silica gel column, eluted with 17% EtOAc in petroleum ether) to afford tert-butyl (R)-4-(4-(2,6-bis(benzyloxy)pyridin-3-yl)phenyl)-3-methylpiperazine-1-carboxylate (300 mg, 525.01 μmol, 48.32% yield, 99% purity) as a yellow oil. MS (M+H)⁺=566.3.

### Step 2. Synthesis of tert-butyl (3R)-4-(4-(2,6-dioxopiperidin-3-yl)phenyl)-3-methylpiperazine-1-carboxylate (3)

To a mixture of Pd/C (55.87 mg, 52.50 μmol, 10% purity), Pd(OH)₂/C (40 mg, 52.50 μmol, 20% purity) in THF (10 mL)was added AcOH (31.53 mg, 525.01 μmol), tert-butyl (R)-4-(4-(2,6-bis(benzyloxy)pyridin-3-yl)phenyl)-3-methylpiperazine-1-carboxylate (300 mg, 525.01 μmol) under Ar atmosphere. The suspension was degassed and purged with H₂ for 3 times. The mixture was stirred under H₂ (50 Psi) atmosphere at 30° C. for 16 hr. LCMS showed one main peak and desired mass was detected. The reaction mixture was filtered directly. The filter cake was washed with THF (3×30 mL). The combined organic phase was concentrated under reduced pressure to afford tert-butyl (3R)-4-(4-(2,6-dioxopiperidin-3-yl)phenyl)-3-methylpiperazine-1-carboxylate (210 mg, 504.04 μmol, 96.00% yield, 93% purity) as a white solid. MS(M+Na)+=410.3.

### Step 3. Synthesis of 3-(4-((R)-2-methylpiperazin-1-yl)phenyl)piperidine-2,6-dione (5)

To a solution of tert-butyl (3R)-4-(4-(2,6-dioxopiperidin-3-yl)phenyl)-3-methylpiperazine-1-carboxylate (210 mg, 504.04 μmol) in dioxane (1 mL) was added HCl/dioxane (2 M, 15 mL). The mixture was stirred at 20° C. for 2 hr. LCMS showed one main peak with desired mass was detected. The mixture was concentrated to afford 3-(4-((R)-2-methylpiperazin-1-yl)phenyl)piperidine-2,6-dione (180 mg, crude, HCl) as a white solid. MS(M+H)⁺=288.1.

### Step 4. Synthesis of tert-butyl (4-(2-((3R)-4-(4-(2,6-dioxopiperidin-3-yl)phenyl)-3-methylpiperazin-1-yl)ethyl)piperidin-1-yl)carbamate (7)

To a solution of 3-(4-((R)-2-methylpiperazin-1-yl)phenyl)piperidine-2,6-dione (180 mg, 555.87 μmol, HCl) in DCM (10 mL) was added TEA (281.24 mg, 2.78 mmol, 386.85 μL) and tert-butyl (4-(2-oxoethyl)piperidin-1-yl)carbamate (161.63 mg, 667.04 μmol) at 15° C. The mixture was stirred at 15° C. for 30 min, followed by the addition of NaBH(OAc)₃ (353.43 mg, 1.67 mmol) in portions. The mixture was stirred at 15° C. for 16 hr. LCMS showed one main peak with desired mass was detected. The mixture was poured into water (30 mL) and then extracted with DCM (30 mL×2). The combined organic phases were dried over anhydrous Na₂SO₄, filtered and concentrated under vacuum. The residue was purified by flash silica gel chromatography (Biotage, 12 g silica gel column, eluted with 53% EtOAc in MeOH) to afford tert-butyl (4-(2-((3R)-4-(4-(2,6-dioxopiperidin-3-yl)phenyl)-3-methylpiperazin-1-yl)ethyl)piperidin-1-yl)carbamate (110 mg, 171.32 μmol, 30.82% yield, 80% purity) as a light yellow solid. MS(M+H)⁺=514.4.

### Step 5. Synthesis of 3-(4-((R)-4-(2-(1-aminopiperidin-4-yl)ethyl)-2-methylpiperazin-1-yl)phenyl)piperidine-2,6-dione (8)

To a solution of tert-butyl (4-(2-((3R)-4-(4-(2,6-dioxopiperidin-3-yl)phenyl)-3-methylpiperazin-1-yl)ethyl)piperidin-1-yl)carbamate (80 mg, 124.59 μmol) in DCM (2 mL) was added TFA (2.46 g, 21.54 mmol). The mixture was stirred at 15° C. for 2 hr. LCMS showed ~93% peak with desired mass. The mixture was concentrated to afford 3-(4-((R)-4-(2-(1-aminopiperidin-4-yl)ethyl)-2-methylpiperazin-1-yl)phenyl)piperidine-2,6-dione (160 mg, crude, 2TFA) was obtained as a yellow oil. MS(M+H)⁺=414.2.

### Step 6. Synthesis of N-(2-chlorophenyl)-4-((2-((4-((4-(2-((3R)-4-(4-(2,6-dioxopiperidin-3-yl)phenyl)-3-methylpiperazin-1-ylethyl)piperidin-1-yl)carbamoyl)phenyl)amino)-5-fluoropyrimidin-4-yl) amino)benzamide (Compound 116)

A mixture of 3-(4-((R)-4-(2-(1-aminopiperidin-4-yl)ethyl)-2-methylpiperazin-1-yl)phenyl)piperidine-2,6-dione (160 mg, 249.38 μmol, 2TFA), 4-((4-((4-((2-chlorophenyl)carbamoyl)phenyl)amino)-5-fluoropyrimidin-2-yl)amino)benzoic acid (114.59 mg, 191.83 μmol), EDCI (73.55 mg, 383.66 μmol), HOBt (51.84 mg, 383.66 μmol) and DIPEA (123.96 mg, 959.14 μmol) in DMF (5 mL), then the mixture was stirred at 15° C. for 16 hr. LCMS showed ~27% peak with desired mass. The mixture was diluted with water (15 mL) and extracted with EtOAc (15 mL×3). The combined organic phase were washed with water (15 mL×3) and saturated NaHCO₃ aqueous solution (15 mL). The organic phase was dried over anhydrous Na₂ SO₄, filtered and concentrated under reduced pressure. The residue was purified by flash silica gel chromatography (Biotage, 4 g silica gel column, eluted with 15% MeOH in DCM) to afford the crude product (20 mg). The crude product was purified by prep-HPLC (neutral condition, column: Waters Xbridge 150*25 mm*5 um; mobile phase: [water(NH₄HCO₃)-ACN]; gradient: 38%-68% B over 9 min) and lyophilized to afford N-(2-chlorophenyl)-4-((2-((4-((4-(2-((3R)-4-(4-(2,6-dioxopiperidin-3-yl)phenyl)-3-methylpiperazin-1-yl)ethyl)piperidin-1-yl) carbamoyl)phenyl)amino)-5-fluoropyrimidin-4-yl)amino)benzamide (2.4 mg, 2.47 μmol, 1.29% yield, 90% purity) as a white solid. MS(M+H)⁺=873.5.

¹H NMR (400 MHz, DMSO-d₆) δ=10.77 (s, 1H), 9.92 (s, 1H), 9.73 (s, 1H), 9.60 (s, 1H), 9.12 (s, 1H), 8.24 (d, J=3.6 Hz, 1H), 7.96-8.05 (m, 4H), 7.76 (d, J=9.2 Hz, 2H), 7.69 (d, J=8.8 Hz, 2H), 7.65 (dd, J=8.0, 1.2 Hz, 1H), 7.56 (dd, J=8.0, 1.2 Hz, 1H), 7.36-7.43 (m, 1H), 7.26-7.32 (m, 1H), 7.04 (d, J=8.8 Hz, 2H), 6.84 (br d, J=8.6 Hz, 2H), 3.95 (br d, J=5.2 Hz, 1H), 3.72 (dd, J=10.8, 4.8 Hz, 1H), 2.77-3.11 (m, 6H), 2.64-2.73 (m, 4H), 2.22-2.35 (m, 4H), 1.98-2.14 (m, 4H), 1.75-1.68 (m, 2H), 1.45-1.38 (m, 2H), 1.27-1.33 (m, 2H), 1.00 (d, J=6.4 Hz, 2H).

### Example 117. Synthesis of N-(2-chlorophenyl)-4-((2-((4-((4-(2-(4-(4-((2,6-dioxopiperidin-3-yl)amino)-2-fluoro-5-methoxyphenyl)piperazin-1-yl)ethyl)piperidin-1-yl)carbamoyl)phenyl)amino)-5-fluoropyrimidin-4-yl)amino)benzamide (Compound 117)

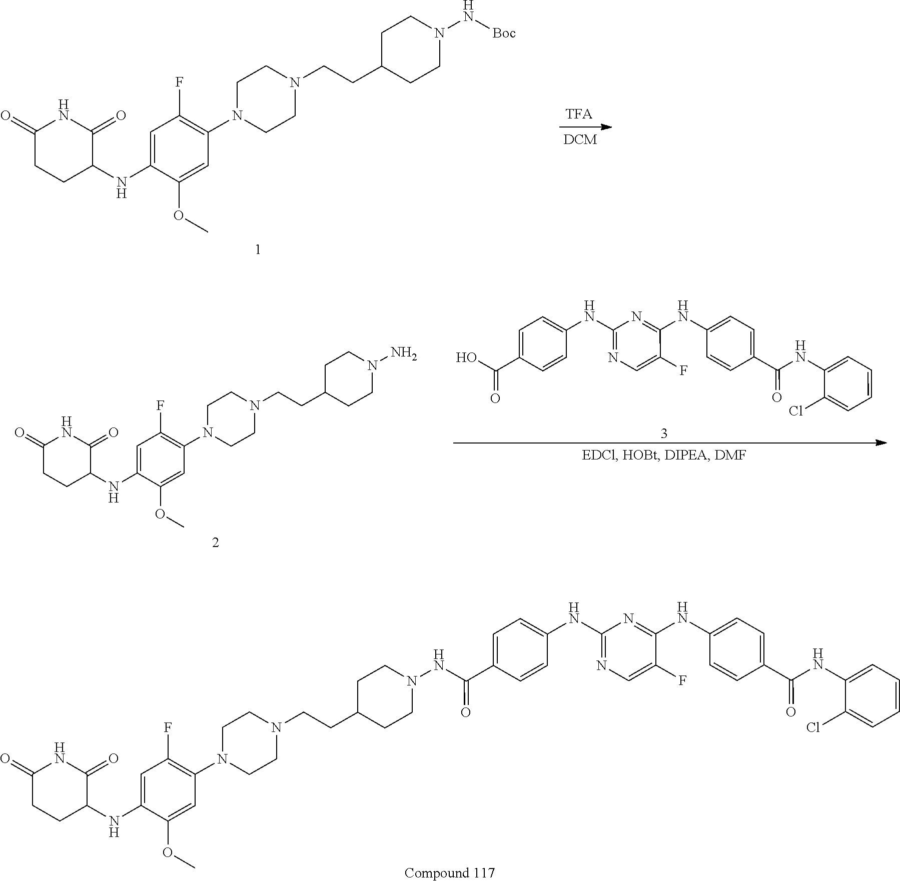

### Step 1. Synthesis of 3-(4((4-(2-(1-aminopiperidin-4-yl)ethyl)piperazin-1-yl)-5-fluoro-2-methoxyphenyl)amino))piperidine-2,6-dione (2)

To a solution of tert-butyl (4-(2-(4-(4-((2,6-dioxopiperidin-3-yl)amino)-2-fluoro-5-methoxyphenyl)piperazin-1-yl)ethyl)piperidin-1-yl)carbamate (80 mg, 142.18 μmol) in DCM (2 mL) was added TFA (3.07 g, 26.92 mmol). The mixture was stirred at 15° C. for 2 hr. LCMS showed ~96% peak with desired mass. The mixture was concentrated under reduced pressure to afford 3-((4-(4-(2-(1-aminopiperidin-4-yl)ethyl)piperazin-1-yl)-5-fluoro-2-methoxyphenyl)amino)piperidine-2,6-dione (160 mg, crude, 2TFA) as a blue solid. MS (M+H)⁺=463.2.

### Step 2. Synthesis of N-(2-chlorophenyl)-4-((2-((4-((4-(2-(4-(4-((2,6-dioxopiperidin-3-yl)amino)-2-fluoro-5-m ethoxyphenyl)piperazin-1-yl)ethyl)piperidin-1-yl)carbamoyl)phenyl)amino)-5-fluoropyrimidin-4-yl)amino)benzamide (Compound 117)

A mixture of 3-((4-(4-(2-(1-aminopiperidin-4-yl)ethyl)piperazin-1-yl)-5-fluoro-2-methoxyphenyl) amino)piperidine-2,6-dione (160 mg, 231.68 μmol, 2TFA), 4-((4-((4-((2-chlorophenyl)carbamoyl)phenyl)amino)-5-fluoropyrimidin-2-yl)amino)benzoic acid (106.46 mg, 178.22 μmol), EDCI (68.33 mg, 356.43 μmol), HOBt (48.16 mg, 356.43 μmol) and DIPEA (115.17 mg, 891.08 μmol) in DMF (5 mL) was stirred at 15° C. for 16 hr. LCMS showed ~37.7% peak with desired mass was detected. The mixture was diluted with water (20 mL) and extracted with EtOAc (10 mL×3). The organic phase was washed with water (10 mL×3) and saturated NaHCO₃ aqueous solution (10 mL). The organic phase was dried over anhydrous Na₂SO₄, filtered and concentrated under reduced pressure. The residue was purified by prep-HPLC (column: Waters Xbridge 150*25 mm*5 um; mobile phase: [water(NH4HCO3)-ACN]; gradient: 38%-68% B over 9 min) and lyophilized to afford the crude product (25 mg, 76% purity). The crude product was further purified by prep-HPLC (TFA condition; column: Phenomenex Luna C18 150*25 mm*10 um; mobile phase: [water(TFA)-ACN]; gradient: 18%-48% B over 11 min) and lyophilized to afford N-(2-chlorophenyl)-4-((2-((4-((4-(2-(4-(4-((2,6-dioxopiperidin-3-yl)amino)-2-fluoro-5-m ethoxyphenyl)piperazin-1-yl)ethyl)piperidin-1-yl)carbamoyl)phenyl)amino)-5-fluoropyrimidin-4-yl)amino)benzamide (6.0 mg, 5.04 μmol, 24.48% yield, 96.7% purity, 2TFA) as a white solid. MS(M+H)⁺=922.3.

¹H NMR (400 MHz, DMSO-d₆) δ=10.86 (s, 1H), 9.94 (s, 1H), 9.77 (s, 1H), 9.65 (s, 1H), 9.28-9.43 (m, 2H), 8.26 (d, J=3.6 Hz, 1H), 7.98-8.08 (m, 4H), 7.80-7.75 (m, 2H), 7.71 (br d, J=8.8 Hz, 2H), 7.66 (d, J=6.8 Hz, 1H), 7.56-7.59 (m, 1H), 7.38-7.43 (m, 1H), 7.30-7.34 (m, 1H), 6.57-6.64 (m, 2H), 4.26-4.34 (m, 1H), 3.82 (s, 3H), 3.60-3.55 (m, 2H), 3.18-3.24 (m, 4H), 2.94-3.11 (m, 6H), 2.74-2.85 (m, 3H), 2.57-2.60 (m, 1H), 2.10-2.16 (m, 1H), 1.92-2.02 (m, 1H), 1.70-1.77 (m, 2H), 1.60-1.68 (m, 2H), 1.30-1.42 (m, 3H).

### Example 118. Synthesis of N-(2-chlorophenyl)-4-((2-((4-((4-(2-(4-(4-(2,6-dioxopiperidin-3-yl)-2-fluoro-5-methoxyphenyl)piperazin-1-yl)ethyl)piperidin-1-yl)carbamoyl)phenyl)amino)-5-fluoropyrimidin-4-yl)amino)benzamide (Compound 118)

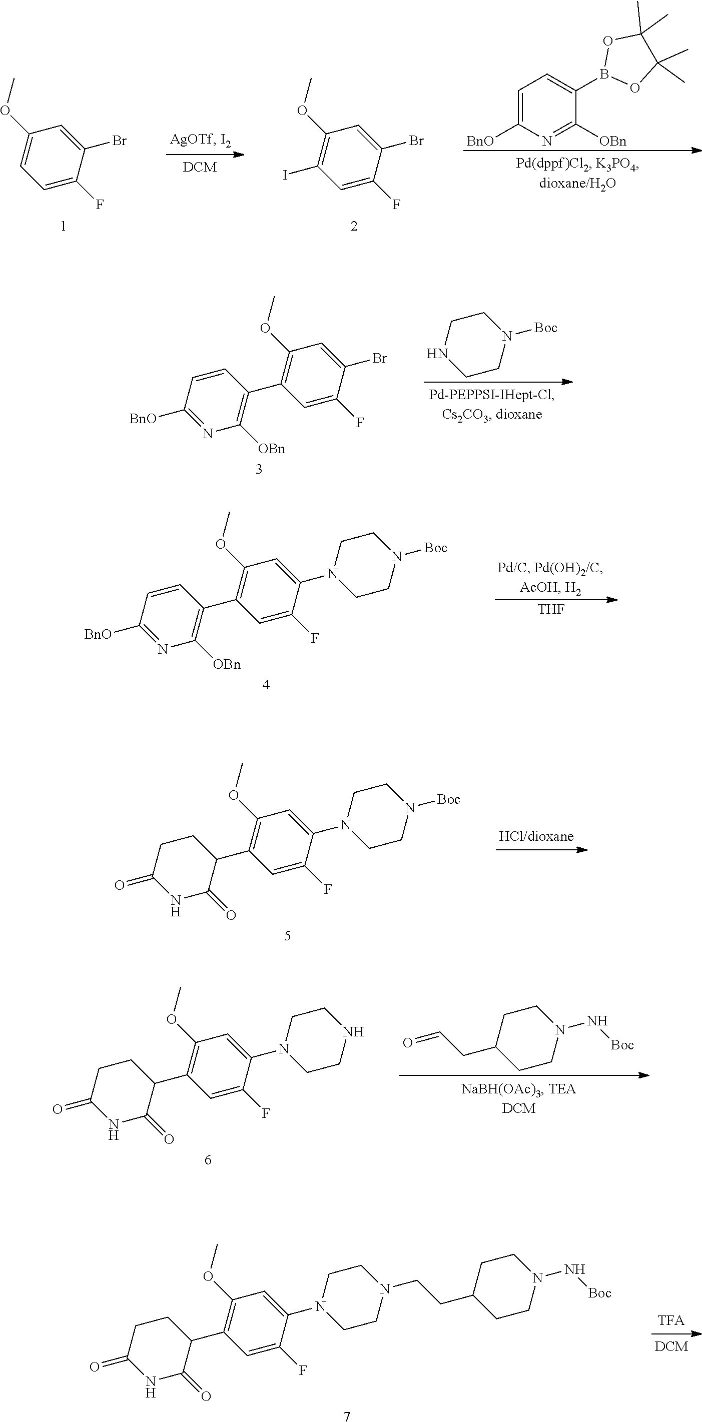

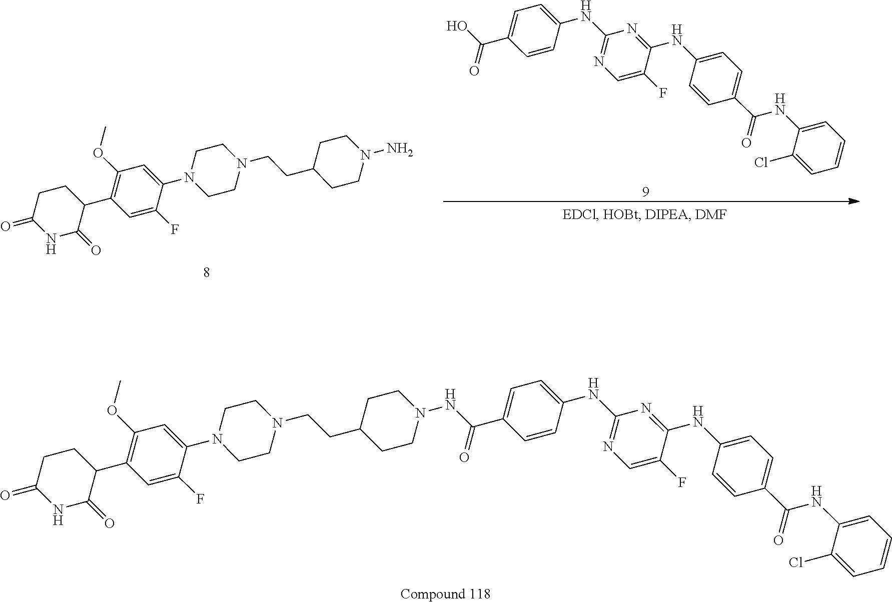 [SMILES: COc1cc(N2CCN(CCC3CCN(N)CC3)CC2)c(F)cc1C1CCC(=O)NC1=O ; O=C(O)c1ccc(Nc2ncc(F)c(Nc3ccc(C(=O)Nc4ccccc4Cl)cc3)n2)cc1 ; "9" ; "8" ; COc1cc(N2CCN(CCC3CCN(NC(=O)c4ccc(Nc5ncc(F)c(Nc6ccc(C(=O)Nc7ccccc7Cl)cc6)n5)cc4)CC3)CC2)c(F)cc1C1CCC(=O)NC1=O ; "Compound 118"]

### Step 1. Synthesis of 1-bromo-2-fluoro-4-iodo-5-methoxybenzene (2)

To a solution of 2-bromo-1-fluoro-4-methoxybenzene (5 g, 24.39 mmol) in DCM (50 mL) was added AgOTf (6.89 g, 26.83 mmol) at 20° C. and the mixture was stirred for 30 min. I₂ (6.81 g, 26.83 mmol) was added to the reaction and the mixture was stirred at 20° C. for 16 hr. LCMS indicated the raw material was consumed completely. The mixture was poured into sat. aq. Na₂S₂O₃ (100 mL) slowly and a lot of solid separated out from the solution. The mixture was filtered and the filter cake was washed with DCM (10 mL×3). The filtrate was extracted with DCM (80 mL×2). The combined organic phases were dried over anhydrous Na₂SO₄, filtered and concentrated under vacuum. The crude was purified by flash silica gel chromatography (Biotage; 40 g SepaFlash® Silica Flash Column, Eluent of 5% EtOAc/Petroleum ether, gradient @ 60 mL/min) to afford 1-bromo-2-fluoro-4-iodo-5-methoxybenzene (4.75 g, 14.35 mmol, 58.86% yield) as a white solid.

¹H NMR (400 MHz, CDCl₃) δ=7.55 (d, J=7.6 Hz, 1H), 6.95 (d, J=6.0 Hz, 1H), 3.87 (s, 3H).

### Step 2. Synthesis of 2,6-bis(benzyloxy)-3-(4-bromo-5-fluoro-2-methoxyphenyl)pyridine (3)

To a stirred mixture of 1-bromo-2-fluoro-4-iodo-5-methoxybenzene (1 g, 3.02 mmol) and 2,6-bis(benzyloxy)-3-(4,4,5,5-tetramethyl-1,3,2-dioxaborolan-2-yl)pyridine (1.26 g, 3.02 mmol) in dioxane (20 mL) and H₂O (2 mL) was added Pd(dppf)Cl₂·CH₂Cl₂ (246.78 mg, 302.19 μmol) and K₃PO₄ (1.92 g, 9.07 mmol) at 20° C. under N₂ atmosphere. The resulting mixture was stirred for 16 hr at 100° C. under N₂ atmosphere. LCMS indicated major peak (~59%) with desired mass detected. The mixture was filtered and the filter cake was washed with EA (20 mL×2). The filtrate was diluted with water (80 mL) and the solution was extracted with EtOAc (80 mL×2). The combined organic phases were dried over anhydrous Na₂SO₄, filtered and concentrated under vacuum. The residue was purified by flash silica gel chromatography (Biotage; 20 g SepaFlash® Silica Flash Column, Eluent of 0-1% EtOAc/Petroleum ether, gradient @ 60 mL/min) to afford 2,6-bis(benzyloxy)-3-(4-bromo-5-fluoro-2-methoxyphenyl)pyridine (1 g, 1.86 mmol, 61.59% yield, 92% purity) as colorless oil. MS(M+H)⁺=496.2.

¹H NMR (400 MHz, CDCl₃) δ=7.50 (d, J=8.0 Hz, 1H), 7.44-7.28 (m, 10H), 7.10 (d, J=8.8 Hz, 1H), 7.06 (d, J=6.0 Hz, 1H), 6.45 (d, J=8.4 Hz, 1H), 5.39 (s, 2H), 5.36 (s, 2H), 3.69 (s, 3H).

### Step 3. Synthesis of tert-butyl 4-(4-(2,6-bis(benzyloxy)pyridin-3-yl)-2-fluoro-5-methoxyphenyl)piperazine-1-carboxy late (4)

To a solution of 2,6-bis(benzyloxy)-3-(4-bromo-5-fluoro-2-methoxyphenyl)pyridine (780 mg, 1.45 mmol), tert-butyl piperazine-1-carboxylate (324.43 mg, 1.74 mmol) in dioxane (10 mL) was added Pd-PEPPSI-IHept-Cl (141.21 mg, 145.16 μmol) and Cs₂ CO₃ (1.42 g, 4.35 mmol) under N₂ atmosphere. The suspension was degassed and purged with N₂ for 3 times. The mixture was stirred under N₂ atmosphere at 100° C. for 16 hr. LCMS indicated major peak (78%) with desired mass detected. The mixture was poured into water (50 mL) and the solution was extracted with EtOAc (50 mL×2). The combined organic phases were dried over anhydrous Na₂SO₄, filtered and concentrated under vacuum. The residue was purified by flash silica gel chromatography (Biotage; 20 g SepaFlash® Silica Flash Column, Eluent of 10% EtOAc/Petroleum ether, gradient @ 60 mL/min) to afford tert-butyl 4-(4-(2,6-bis(benzyloxy)pyridin-3-yl)-2-fluoro-5-methoxyphenyl)piperazine-1-carboxy late (753 mg, 1.22 mmol, 83.91% yield, 97% purity) as a yellow solid. MS(M+H)+=600.4.

¹H NMR (400 MHz, DMSO-d₆) δ=7.53 (br d, J=8.0 Hz, 1H), 7.42-7.23 (m, 10H), 7.01 (br d, J=12.4 Hz, 1H), 6.66 (br d, J=7.2 Hz, 1H), 6.47 (br d, J=8.4 Hz, 1H), 5.33 (br d, J=5.2 Hz, 4H), 3.67 (s, 3H), 3.55-3.42 (m, 4H), 3.10-2.95 (m, 4H), 1.42 (s, 9H).

### Step 4. Synthesis of tert-butyl 4-(4-(2,6-dioxopiperidin-3-yl)-2-fluoro-5-methoxyphenyl)piperazine-1-carboxylate (5)

To a mixture of Pd/C (130 mg, 122.16 μmol, 10% purity), Pd(OH)₂/C (130 mg, 20% purity) in THF (10 mL) was added AcOH (73.14 mg, 1.22 mmol), tert-butyl 4-(4-(2,6-bis(benzyloxy)pyridin-3-yl)-2-fluoro-5-methoxyphenyl)piperazine-1-carboxy late (753 mg, 1.22 mmol) under Ar atmosphere. The suspension was degassed and purged with H₂ for 3 times. The mixture was stirred under H₂ (50 Psi) atmosphere at 26° C. for 48 hr. LCMS indicated major peak (96%) with desired mass detected. The reaction mixture was filtered. The filter cake was washed with THF (3×30 mL). The combined organic phases were concentrated under reduced pressure to afford tert-butyl 4-(4-(2,6-dioxopiperidin-3-yl)-2-fluoro-5-methoxyphenyl)piperazine-1-carboxylate (413 mg, 911.33 μmol, 74.82% yield, 93% purity) as a white solid. MS(M−56+H)+=366.1.

¹H NMR (400 MHz, DMSO-d₆) δ=10.72 (s, 1H), 6.97 (d, J=12.8 Hz, 1H), 6.64 (d, J=7.2 Hz, 1H), 3.86 (dd, J=12.0, 5.2 Hz, 1H), 3.73 (s, 3H), 3.55-3.45 (m, 4H), 3.02-2.96 (m, 4H), 2.71-2.62 (m, 1H), 2.50-2.45 (m, 1H), 2.23-2.13 (m, 1H), 1.88-1.84 (m, 1H), 1.42 (s, 9H).

### Step 5. Synthesis of 3-(5-fluoro-2-methoxy-4-(piperazin-1-yl)phenyl)piperidine-2,6-dione (6)

A solution of tert-butyl 4-(4-(2,6-dioxopiperidin-3-yl)-2-fluoro-5-methoxyphenyl)piperazine-1-carboxylate (413 mg, 911.33 μmol) in HCl/dioxane (10 mL, 2M) was stirred at 20° C. for 2 hr. LCMS indicated major peak (84%) with desired mass detected. The mixture was concentrated under vacuum to afford 3-(5-fluoro-2-methoxy-4-(piperazin-1-yl)phenyl)piperidine-2,6-dione (400 mg, crude, HCl) as a yellow solid. MS(M+H)⁺=322.1.

### Step 6. Synthesis of tert-butyl (4-(2-(4-(4-(2,6-dioxopiperidin-3-yl)-2-fluoro-5-methoxyphenyl)piperazin-1-yl)ethyl)piperidin-1-yl)carbamate (7)

To a solution of 3-(5-fluoro-2-methoxy-4-(piperazin-1-yl)phenyl)piperidine-2,6-dione (400 mg, 1.12 mmol, HCl) in DCM (10 mL) was added TEA (451.00 mg, 4.46 mmol) and then followed by a solution of tert-butyl (4-(2-oxoethyl)piperidin-1-yl)carbamate (240 mg, 891.41 μmol) in DCM (2 mL). After stirring at 20° C. for 30 min, NaBH(OAc)₃ (566.78 mg, 2.67 mmol) was added at 20° C. and the resulting mixture was stirred at 20° C. for 16 h. LCMS indicated ~13% of the amine was remained and ~58% peak of the desired mass detected. The mixture was poured into water (20 mL) and the solution was extracted with DCM (20 mL×2). The combined organic phases were dried over anhydrous Na₂SO₄, filtered and concentrated under vacuum. The residue was purified by flash silica gel chromatography (Biotage; 12 g SepaFlash® Silica Flash Column, Eluent of 60% EtOAc/MeOH, gradient @ 60 mL/min) to afford tert-butyl (4-(2-(4-(4-(2,6-dioxopiperidin-3-yl)-2-fluoro-5-methoxyphenyl)piperazin-1-yl)ethyl)piperidin-1-yl)carbamate (335 mg, 538.29 μmol, 60.39% yield, 88% purity) as yellow oil. MS(M+H)⁺=548.3.

### Step 7. Synthesis of 3-(4-(4-(2-(1-aminopiperidin-4-yl)ethyl)piperazin-1-yl)-5-fluoro-2-methoxyphenyl)piperidine-2,6-dione (8)

To a solution of tert-butyl (4-(2-(4-(4-(2,6-dioxopiperidin-3-yl)-2-fluoro-5-methoxyphenyl)piperazin-1-yl)ethyl)piperidin-1-yl)carbamate (315 mg, 506.15 μmol) in DCM (6 mL) was added TFA (2 mL) at 20° C. The mixture was stirred at 20° C. for 1 hr. LCMS indicated major peak (93%) with desired mass detected. The mixture was concentrated under vacuum to afford 3-(4-(4-(2-(1-aminopiperidin-4-yl)ethyl)piperazin-1-yl)-5-fluoro-2-methoxyphenyl)piperidine-2,6-dione (460 mg, crude, TFA) as yellow oil. MS(M+H)⁺=448.3.

### Step 8. Synthesis of N-(2-chlorophenyl)-4-((2-((4-((4-(2-(4-(4-(2,6-dioxopiperidin-3-yl)-2-fluoro-5-methoxyphenyl)piperazin-1-yl)ethyl)piperidin-1-yl)carbamoyl)phenyl)amino)-5-fluoropyrimidin-4-yl)amino)benzamide (Compound 118)

To a solution of 3-(4-(4-(2-(1-aminopiperidin-4-yl)ethyl)piperazin-1-yl)-5-fluoro-2-methoxyphenyl)piperidine-2,6-dione (230 mg, 409.57 μmol, TFA) in DMF (10 mL) was added DIPEA (135.23 mg, 1.05 mmol), HOBt (56.55 mg, 418.52 μmol), EDCI (80.23 mg, 418.52 μmol) and 4-((4-((4-((2-chlorophenyl)carbamoyl)phenyl)amino)-5-fluoropyrimidin-2-yl)amino)benzoic acid (100 mg, 209.26 μmol) at 20° C. The mixture was stirred at 20° C. for 16 hr. LCMS indicated the reaction completed. The mixture was diluted with EA (80 mL) and washed with water (20 mL×3) and sat. aq. NaHCO₃ (30 mL). The organic phase was dried over anhydrous Na₂SO₄, filtered and concentrated under vacuum. The crude was purified by flash silica gel chromatography (Biotage; 12 g SepaFlash® Silica Flash Column, Eluent of 50% EtOAc/MeOH, gradient @ 30 mL/min) to afford the residue. The residue was purified by prep-HPLC (column: Phenomenex Luna C18 150*25 mm*10 um; mobile phase: [water(TFA)-ACN]; gradient: 18%-48% B over 11 min) and lyophilized to afford N-(2-chlorophenyl)-4-((2-((4-((4-(2-(4-(4-(2,6-dioxopiperidin-3-yl)-2-fluoro-5-methoxyphenyl)piperazin-1-yl)ethyl)piperidin-1-yl)carbamoyl)phenyl)amino)-5-fluoropyrimidin-4-yl)amino)benzamide (13.9 mg, 12.00 μmol, 5.73% yield, 98% purity, 2TFA) as a white solid. MS(M+H)⁺=907.2.

¹H NMR (400 MHz, DMSO-d₆) δ=10.74 (s, 1H), 9.95-9.94 (m, 1H), 9.77-9.76 (m, 1H), 9.67 (s, 1H), 9.65-9.55 (m, 2H), 8.26-8.23 (m, 1H), 8.04-8.00 (m, 4H), 7.83-7.70 (m, 4H), 7.66-7.64 (m, 1H), 7.57 (dd, J=8.0, 1.2 Hz, 1H), 7.40 (dt, J=7.6, 1.2 Hz, 1H), 7.32-7.28 (m, 1H), 7.03 (d, J=13.2 Hz, 1H), 6.66 (br d, J=7.2 Hz, 1H), 3.88 (dd, J=12.0, 4.8 Hz, 1H), 3.77 (s, 3H), 3.61-3.54 (m, 4H), 3.22-3.05 (m, 8H), 2.85-2.79 (m, 2H), 2.72-2.65 (m, 1H), 2.49-2.46 (m, 2H), 2.25-2.14 (m, 1H), 1.88-1.84 (m, 1H), 1.75-1.74 (m, 2H), 1.71-1.65 (m, 2H), 1.46-1.15 (m, 2H).

### Example 119. Synthesis of 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)-2-fluoro-5-methoxyphenyl)piperazin-1-yl)ethyl)piperidin-1-yl)-2-fluorobenzamide (Compound 119)

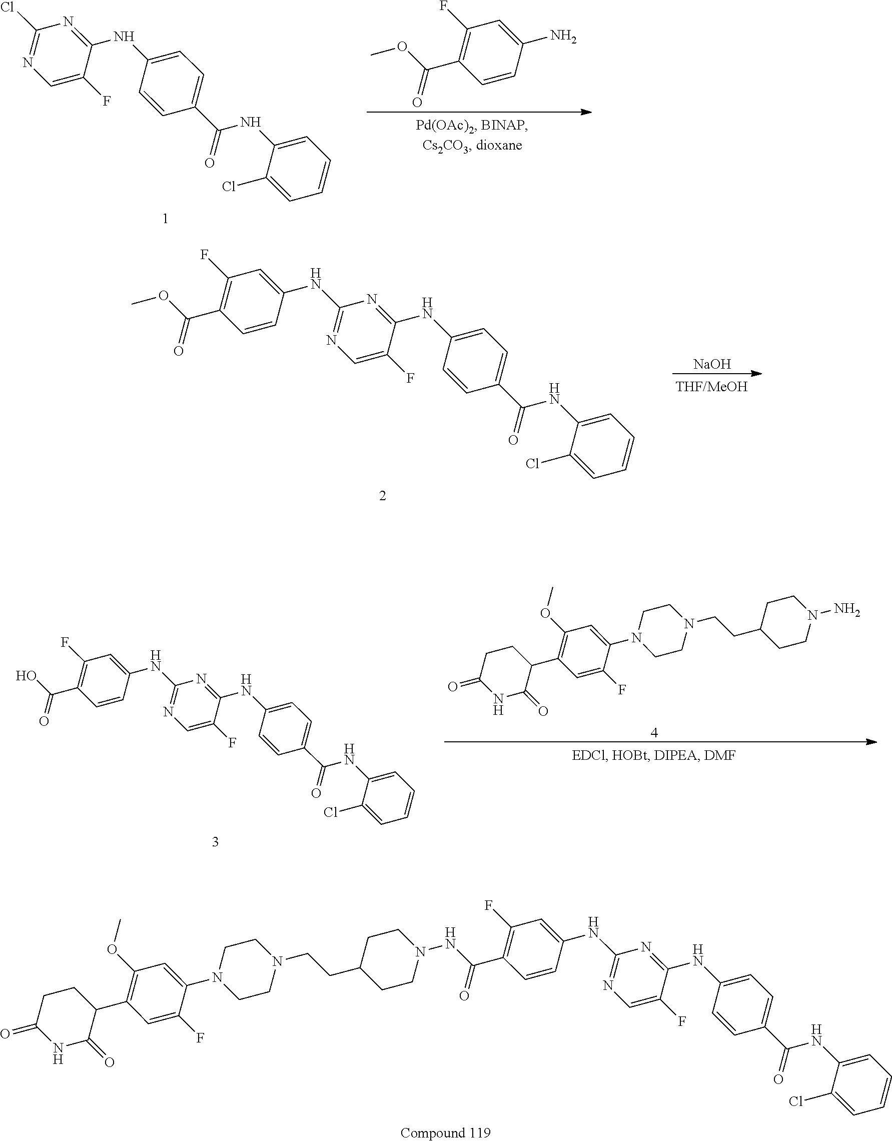

### Step 1. Synthesis of methyl 4-((4-((4-((2-chlorophenyl)carbamoyl)phenyl)amino)-5-fluoropyrimidin-2-yl)amino)-2-fluorobenzoate (2)

To a mixture of 4-((2-chloro-5-fluoropyrimidin-4-yl)amino)-N-(2-chlorophenyl)benzamide (1 g, 2.49 mmol), methyl 4-amino-2-fluorobenzoate (379.38 mg, 2.24 mmol) and Cs₂CO₃ (2.03 g, 6.23 mmol) in dioxane (20 mL) was added Pd(OAc)₂ (27.97 mg, 124.60 μmol) and BINAP (155.17 mg, 249.21 μmol) at 20° C. under N₂ atmosphere, the resulting mixture was stirred at 100° C. for 14 hrs under N₂ atmosphere. LCMS showed a main peak with desired mass. The mixture was diluted with EtOAc (50 mL) and filtered. The filtrate was concentrated to afford the crude product. The crude product was purified by trituration from MTBE (20 mL) to afford methyl 4-((4-((4-((2-chlorophenyl)carbamoyl)phenyl)amino)-5-fluoropyrimidin-2-yl)amino)-2-fluorobenzoate (900 mg, 1.68 mmol, 67.29% yield, 95% purity) as a yellow solid. MS(M+H)⁺=510.1.

¹H NMR (400 MHz, DMSO-d₆) δ=9.99 (s, 1H), 9.89 (br s, 2H), 8.30 (d, J=3.6 Hz, 1H), 8.00 (q, J=8.8 Hz, 4H), 7.92 (dd, J=14.8, 2.0 Hz, 1H), 7.79 (t, J=8.8 Hz, 1H), 7.66 (dd, J=8.0, 1.6 Hz, 1H), 7.57 (dd, J=8.0, 1.4 Hz, 1H), 7.47 (dd, J=8.8, 2.0 Hz, 1H), 7.40 (dt, J=7.6, 1.4 Hz, 1H), 7.29 (dt, J=7.6, 1.6 Hz, 1H), 3.80 (s, 3H).

### Step 2. Synthesis of 4-((4-((4-((2-chlorophenyl)carbamoyl)phenyl)amino)-5-fluoropyrimidin-2-yl)amino)-2-fluorobenzoic acid (3)

To a solution of methyl 4-((4-((4-((2-chlorophenyl)carbamoyl)phenyl)amino)-5-fluoropyrimidin-2-yl)amino)-2-fluorobenzoate (900 mg, 1.77 mmol) in THF (10 mL) and MeOH (5 mL) was added aqueous NaOH (2 M, 3.53 mL) at 20° C., the resulting mixture was stirred at 45° C. for 14 hrs. LCMS showed the starting material was consumed completely and the desired mass. The mixture was acidified by 2N HCl solution to pH=5 and then filtered. The filter cake was collected and dried to afford 4-((4-((4-((2-chlorophenyl)carbamoyl)phenyl)amino)-5-fluoropyrimidin-2-yl)amino)-2-fluorobenzoic acid (700 mg, 1.27 mmol, 71.98% yield, 90% purity) as a yellow solid. MS(M+H)⁺=496.1.

¹H NMR (400 MHz, DMSO-d₆) δ=12.77-12.69 (m, 1H), 9.96 (d, J=2.0 Hz, 1H), 9.84 (d, J=6.0 Hz, 1H), 8.29 (d, J=3.4 Hz, 1H), 8.05-7.97 (m, 4H), 7.95 (d, J=7.2 Hz, 1H), 7.88 (br d, J=14.8 Hz, 1H), 7.79-7.74 (m, 1H), 7.65 (d, J=7.6 Hz, 1H), 7.56 (d, J=7.6 Hz, 1H), 7.47 (dd, J=8.8, 1.6 Hz, 1H), 7.39 (t, J=7.6 Hz, 1H), 7.32-7.26 (m, 1H).

### Step 3. Synthesis of 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)-2-fluoro-5-methoxyphenyl)piperazin-1-yl)ethyl)piperidin-1-yl)-2-fluorobenzamide (Compound 119)

To a solution of 4-((4-((4-((2-chlorophenyl)carbamoyl)phenyl)amino)-5-fluoropyrimidin-2-yl)amino)-2-fluorobenzoic acid (100 mg, 201.67 μmol) in DMF (3 mL) was added DIPEA (130.32 mg, 1.01 mmol), HOBt (54.50 mg, 403.34 μmol), EDCI (77.32 mg, 403.34 μmol) and 3-(4-(4-(2-(1-aminopiperidin-4-yl)ethyl)piperazin-1-yl)-5-fluoro-2-methoxyphenyl)piperidine-2,6-dione (230 mg, 409.57 μmol, TFA) at 20° C. The mixture was stirred at 20° C. for 16 hr. LCMS indicated the reaction completed. The mixture was diluted with EtOAc (80 mL) and washed with water (20 mL×3) and sat. aq. NaHCO₃ (20 mL). The organic phase was dried over anhydrous Na₂SO₄, filtered and concentrated under vacuum. The crude product was purified by flash silica gel chromatography (Biotage; 12 g SepaFlash® Silica Flash Column, Eluent of 55% EtOAc/MeOH, gradient @ 30 mL/min) to afford the residue. The residue was further purified by prep-HPLC (column: Phenomenex Luna C18 150*25 mm*10 um; mobile phase: [water(TFA)-ACN]; gradient: 18%-48% B over 12 min) and lyophilized to afford 4-((4-((4-((2-chlorophenyl)carbamoyl)phenyl)amino)-5-fluoropyrimidin-2-yl)amino)-N-(4-(2-(4-(4-(2,6-dioxopiperidin-3-yl)-2-fluoro-5-methoxyphenyl)piperazin-1-yl)ethyl)piperidin-1-yl)-2-fluorobenzamide (3.9 mg, 3.31 μmol, 1.64% yield, 98% purity, 2TFA) as a light yellow solid. MS(M+H)⁺=925.2.

¹H NMR (400 MHz, DMSO-d₆) δ=10.74 (s, 1H), 9.92 (s, 1H), 9.79 (br s, 2H), 9.41 (br s, 1H), 8.98 (s, 1H), 8.28 (d, J=3.6 Hz, 1H), 8.03-7.98 (m, 4H), 7.83 (br d, J=13.6 Hz, 1H), 7.65 (br d, J=7.6 Hz, 1H), 7.57 (d, J=8.0 Hz, 1H), 7.48-7.38 (m, 3H), 7.32-7.28 (m, 1H), 7.08-6.95 (m, 1H), 6.66 (br d, J=7.6 Hz, 1H), 3.90-3.85 (m, 1H), 3.76 (s, 3H), 3.61-3.49 (m, 4H), 3.26-3.19 (m, 4H), 3.11-3.01 (m, 4H), 2.72-2.59 (m, 4H), 2.25-2.15 (m, 1H), 1.90-1.80 (m, 1H), 1.74-1.58 (m, 4H), 1.34-1.24 (m, 2H).

### Example 120. Synthesis of N-(2-chlorophenyl)-4-((2-((4-((4-(2-(4-(4-(2,6-dioxopiperidin-3-yl)phenyl)piperazin-1-yl)propyl)piperidin-1-yl)carbamoyl)phenyl)amino)-5-fluoropyrimidin-4-yl)amino)benzamide (Compound 120)

In a manner similar to the other examples obtained the titled compound.

### Example 121. Synthesis of N-(4-(2-(4-(4-(2,6-dioxopiperidin-3-yl)-2-fluoro-5-methoxyphenyl)piperazin-1-yl)eth yl)piperidin-1-yl)-2-fluoro-4-((5-fluoro-4-((4-(2-fluorobenzamido)phenyl)amino)pyrimidin-2-yl)amino)benzamide (Compound 121)

In a manner similar to the other examples obtained the titled compound.

### Example 122. Synthesis of N-(4-((2-((4-((4-(2-(4-(4-(2,6-dioxopiperidin-3-yl)-2,5-difluorophenyl)piperazin-1-yl)ethyl)piperidin-1-yl)carbamoyl)phenyl)amino)-5-fluoropyrimidin-4-yl)amino)phen yl)-2-fluorobenzamide (Compound 122)

In a manner similar to the other examples obtained the titled compound.

### Example 123. Synthesis of N-(4-(2-(4-(4-(2,6-dioxopiperidin-3-yl)phenyl)piperazin-1-yl)ethyl)piperidin-1-yl)-2-fluoro-4-((5-fluoro-4-((4-(2-fluorobenzamido)phenyl)amino)pyrimidin-2-yl)amino) benzamide (Compound 123)

In a manner similar to the other examples obtained the titled compound.

### Comparative Examples

Comparative compounds, e.g., Compounds 3, 4, 11, 17, 18, 19, 20, 23, 24, 31 disclosed in WO 2022/270987 A1 may be synthesized with reference to WO 2022/270987 A1, which is incorporated in the present disclosure.

### Experimental Example

### 1. Luciferase Assay for AURKA

### Preparation and Culture of HeLa LgBit (AURKA-HiBit KI) Cell Line

HeLa cell lines that stably express LgBit were constructed via LgBit vector transfection. Then, donor DNA and guide RNA for insertion of HiBit amino acid sequence at C-terminus of inherent Aurora kinase A (AURKA) genes were designed and constructed. Thereafter, they were transfected into the cell lines with CRISPR/Cas9 expression vector system. HiBit gene knock-in single cells were selected, passaged and used for the experiments.

For cell culture, DMEM (Gibco, Cat. No. 11995-065; Lot. No. 2562497), FBS (Gibco, Cat. No. 16000-044; Lot. No. 2511328P), Penicillin/Streptomycin (PS) (Gibco, Cat. No. 15140-122; Lot. No. 2585636), 90 mm² cell culture dish (SPL, Cat. No. 20100), 96-well white plate, PBS pH 7.4 (Gibco, Cat. No. 12605-010; Lot. No. 2522525), TrypLE™ Express (Gibco, Cat. No. 12605-010; Lot. No. 2522525), counting chamber (hematocytometer) (Hirschmann, Cat. No. 8100204; Lot. No. 8021087) and 0.4% Trypan Blue solution (DYNEBIO, Cat. No. CBT3710; Lot. No. 20231017) were used. Cells were cultured in DMEM supplemented with 10% FBS and 1% PS and incubated in CO₂ incubator (Thermo, Cat. No. 4111) at 37° C., 5% CO₂ condition.

### Luciferase Assay

HeLa LgBit (AURKA-HiBit KI) cell lines were cultured in 90 mm² cell culture dish, and 3×10⁴ cells in 150 g of total medium volume were seeded and incubated for 18 hours in CO₂ incubator. Then, Eudurazine (Promega, Cat. No. N247B; Lot. No. 495218) was added to each well to make up 1% of the total volume, followed by further incubation for 2 hours in CO₂ incubator.

The present compounds were fully dissolved in DMSO (Sigma-Aldrich Cat. No. D2438, Lot. No. RNBL7287) and 10 nM or 100 nM of total concentration were used in the experiment. After adding the compounds in each well of 96-well white plate, the plate was placed in the plate reader (BMG Labtech, CLARIOstar Plus) for real-time analysis. The wavelength for analysis was set to 470-480 nM, and the luminescence value was measured after 12 hours. The luminescence value was analyzed through an Excel program.

The results of luminescence value after 10 nM of the present compounds are shown in Table 6. According to the range of luminescence value, the grades were classified into A, B and C (A is <0.3; B is 0.3 to 0.5; C is 0.5 to 0.7; and D is 0.7 to 0.8).

TABLE 6

| Compound | Level | Compound | Level |
|---|---|---|---|
| 1 | C | 2 | C |
| 3 | C | 4 | C |
| 5 | B | 6 | B |
| 7 | A | 8 | B |
| 9 | B | 10 | D |
| 11 | D | 12 | B |
| 13 | C | 14 | D |
| 15 | D | 16 | B |
| 17 | A | 18 | A |
| 19 | C | 20 | D |
| 21 | B | 22 | A |
| 23 | B | 24 | B |
| 25 | C | 26 | B |
| 27 | B | 28 | C |
| 29 | C | 30 | B |
| 31 | C | 32 | B |
| 33 | B | 34 | B |
| 35 | B | 36 | B |
| 37 | B | 38 | B |
| 39 | B | 40 | B |
| 41 | B | 42 | B |
| 43 | A | 44 | B |
| 45 | C | 46 | C |
| 47 | D | 48 | A |
| 49 | A | 50 | B |
| 51 | A | 52 | A |
| 53 | B | 54 | B |
| 55 | A | 56 | A |
| 57 | A | 58 | A |
| 59 | C | 60 | D |
| 61 | C | 62 | B |
| 63 | A | 64 | B |
| 65 | B | 66 | A |
| 67 | B | 68 | B |
| 69 | A | 70 | A |
| 71 | A | 72 | B |
| 73 | A | 74 | B |
| 75 | A | 76 | B |
| 77 | A | 78 | B |
| 79 | A | 80 | A |
| 81 | A | 82 | A |
| 83 | B | 84 | C |
| 85 | B | 86 | A |
| 87 | B | 88 | A |
| 89 | B | 90 | A |
| 91 | D | 92 | B |
| 93 | A | 94 | A |
| 95 | B | 96 | A |
| 97 | B | 98 | A |
| 99 | D | 100 | C |
| 101 | B | 102 | B |
| 103 | B | 104 | A |
| 105 | B | 106 | A |
| 107 | A | 108 | A |
| 109 | B | 110 | C |
| 111 | A | 112 | A |
| 113 | A | 114 | A |
| 116 | A | 117 | A |
| 118 | B | 119 | C |

The results of luminescence value after 100 nM of the present compounds are shown in Table 7. According to the range of luminescence value, the grades were classified into A, B and C (A is <0.2; B is 0.2 to 0.4; and C is 0.4 to 0.6).

TABLE 7

| Compound | Level | Compound | Level |
|---|---|---|---|
| 1 | A | 2 | B |
| 3 | A | 4 | A |
| 5 | A | 6 | A |
| 7 | A | 8 | A |
| 9 | A | 10 | C |
| 11 | B | 12 | A |
| 13 | B | 14 | C |
| 15 | B | 16 | A |
| 17 | A | 18 | A |
| 19 | B | 20 | C |
| 21 | A | 22 | A |
| 23 | A | 24 | A |
| 25 | B | 26 | B |
| 27 | B | 28 | B |
| 29 | B | 30 | A |
| 31 | B | 32 | B |
| 33 | A | 34 | A |
| 35 | B | 36 | A |
| 37 | B | 38 | B |
| 39 | A | 40 | A |
| 41 | B | 42 | A |
| 43 | A | 44 | A |
| 45 | B | 46 | B |
| 47 | B | 48 | A |
| 49 | A | 50 | A |
| 51 | A | 52 | A |
| 53 | A | 54 | B |
| 55 | A | 56 | A |
| 57 | A | 58 | A |
| 59 | A | 60 | B |
| 61 | A | 62 | A |
| 63 | A | 64 | A |
| 65 | A | 66 | A |
| 67 | A | 68 | A |
| 69 | A | 70 | A |
| 71 | A | 72 | A |
| 73 | A | 74 | A |
| 75 | A | 76 | A |
| 77 | A | 78 | A |
| 79 | A | 80 | A |
| 81 | A | 82 | A |
| 83 | A | 84 | B |
| 85 | A | 86 | A |
| 87 | B | 88 | A |
| 89 | B | 90 | A |
| 91 | C | 92 | A |
| 93 | A | 94 | A |
| 95 | A | 96 | A |
| 97 | A | 98 | A |
| 99 | B | 100 | A |
| 101 | A | 102 | A |
| 103 | A | 104 | A |
| 105 | A | 106 | A |
| 107 | A | 108 | A |
| 109 | A | 110 | B |
| 111 | A | 112 | A |
| 113 | A | 114 | A |
| 116 | A | 117 | A |
| 118 | A | 119 | B |

### 2. Cell Viability Assay for NCI-H446 Cell Line

### Culture of NCI-H446 Cell Line

The NCI-H446 (hereafter H446) cell lines were provided from American Type Culture Collection (ATCC). For cell culture, RPMI medium 1640 (Gibco, Cat. No. 22400-089; Lot. No. 2522717), FBS (Gibco, Cat. No. 16000-044; Lot. No. 2716756P), Penicillin/Streptomycin (PS) (Gibco, Cat. No. 15140-122; Lot. No. 2585636), 75T cell culture flask (SPL, Cat. No. 70075), 96 well cell culture plate (SPL, Cat. No. 30096), PBS pH 7.4 (Gibco, Cat. No. 10010-023; Lot. No. 2561466), TrypLE™ Express (Gibco, Cat. No. 12605-010; Lot. No. 2492811), counting chamber (hematocytometer) (Hirschmann, Cat. No. 8100204) and 0.4% Trypan Blue solution (DYNEBIO, Cat. No. CBT3710; Lot. No. 20231017) were used. Cells were cultured in RPMI 1640 medium supplemented with 10% FBS and 1% PS and incubated at 37° C., 5% CO₂ condition in CO₂ incubator (ThermoScientific, Cat. No. 4111).

### Cell Viability Assay

For experiment, the present compounds were fully dissolved in DMSO (Sigma-Aldrich Cat. No. D2438, Lot. No. RNBL7287.

In each well of 96-well cell culture plate, 6×10¹ cells of H446 cell lines were seeded in 150 μl of total medium volume. After 24 hours of cell seeding, the present compounds were treated.

The present compounds were each diluted 3-folds from the highest concentration of 1000 nM to the lowest concentration of 0.15 nM. The compound was added to each well to make the total volume of 20 μl and the cells were cultured for 5 days in CO₂ incubator (Thermo Fisher Science, Cat. No. 4111).

Thereafter, 20 μl of EZ-Cytox (DOGEN, Cat. NO. EZ-3000, Lot. No. DLS2308) was added to each well and the cells were cultured for 4 hours at the same condition in CO₂ incubator. After culture, the absorbance of the sample was measured by setting the wavelength of a plate reader (BMG Labtech, CLARIOstar Plus) to 450 nM. The samples were shaked for 2 minutes in the plate reader before measurement. The final measured value was analyzed and displayed as a bar graph through an Excel program, and IC₅₀ value was measured.

The results of IC₅₀ value of the present compounds are shown in Table 8. According to the range of value, the grades were classified into A, B and C (A is <50 nM; B is 50 to 100 nM; C is 100 to 150 nM; D is 150 to 200 nM; and E is >200 nM).

TABLE 8

| Compound | Level | Compound | Level |
|---|---|---|---|
| 1 | C | 3 | B |
| 4 | C | 5 | A |
| 6 | A | 7 | A |
| 8 | A | 9 | A |
| 12 | A | 13 | E |
| 16 | B | 17 | B |
| 18 | A | 21 | A |
| 22 | C | 23 | A |
| 24 | A | 26 | A |
| 30 | A | 33 | C |
| 35 | D | 36 | C |
| 38 | E | 43 | A |
| 44 | C | 45 | B |
| 48 | A | 49 | A |
| 50 | B | 51 | A |
| 52 | A | 53 | A |
| 55 | A | 56 | A |
| 57 | C | 58 | A |
| 63 | A | 65 | B |
| 66 | A | 67 | C |
| 69 | B | 70 | B |
| 71 | C | 72 | A |
| 73 | C | 74 | A |
| 75 | B | 76 | B |
| 77 | B | 78 | B |
| 79 | A | 80 | B |
| 81 | A | 82 | A |
| 83 | E | 85 | D |
| 86 | B | 88 | A |
| 90 | A | 93 | A |
| 94 | A | 95 | B |
| 96 | A | 97 | B |
| 98 | C | 99 | E |
| 100 | E | 101 | E |
| 103 | B | 104 | A |
| 106 | C | 108 | A |
| 109 | B | 110 | D |
| 111 | A | 112 | A |
| 113 | A | 114 | A |
| 116 | A | 117 | A |
| 118 | B | | |

1. A compound represented by the following Formula I: 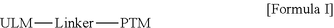
wherein:
PTM is Aurora kinase A (AURKA) binding moiety represented by the following Formula II: 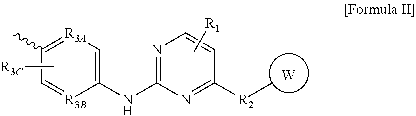
wherein:
R₁ is halo;
R₂ is —NH— or —O—;
R_{3A} and R_{3B} are each independently —CH— or —N—;
R_{3C} is H, halo or OC₁₋₃alkyl; 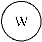
is 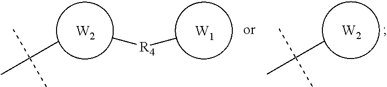
R₄ is —CO—, —CONH—, —CON(CH₃)— or —NHCO—; 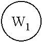
is 5- to 6-membered cycloalkyl, heterocyclyl, aryl or heteroaryl optionally substituted with 1 to 3 halo; 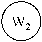
is phenyl or 5- to 6-membered heteroaryl optionally substituted with halo or C₁₋₃alkyl; and [symbol]
is a covalent bond connected to Linker; and
ULM is CRBN E3 ubiquitin ligase binding moiety represented by the following Formula III-1, III-2A or III-2B: 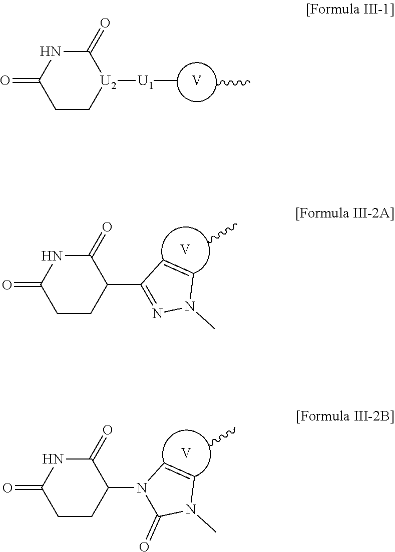
wherein:
U₁ is a covalent bond, —NH—, —N(CH₃)—, —NHCO— or —CONH—;
U₂ is CH or N; 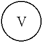
is phenyl or 6-membered heteroaryl optionally substituted with 1 to 3 halo, C₁₋₃alkyl or OC₁₋₃alkyl; and [symbol]
is a covalent bond connected to Linker; and
Linker is a linking group represented by the following Formula IV: 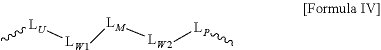
wherein:
L_{U} is a covalent bond, —CH₂—, —NH—, —O—, CH₂CH₂—, —NHCH₂—, —CH₂NH—, —OCH₂—, —CH₂O—, —CH═CH— or —CC—;
L_{M} is a covalent bond, —CH₂—, —CH₂CH₂—, —CH(CH₃)CH₂—, —CH₂CH(CH₃)—, —CH(CH₂CH₃)CH₂—, —CH₂CH(CH₂CH₃)—, —COCH₂—, —CH₂CO—, —CH₂NH—, —NHCH₂—, —CH₂N(CH₃)—, —N(CH₃)CH₂—, —CH₂CH₂CH₂—, —COCH₂CH₂—, —CH₂COCH₂—, —CH₂CH₂CO—, —NHCH₂CH₂—, —CH₂NHCH₂—, —CH₂CH₂NH—, —N(CH₃)CH₂CH₂—, —CH₂N(CH₃)CH₂— or —CH₂CH₂N(CH₃)—;
L_{P} is —CH₂—, —O—, —CH₂O—, —OCH₂—, —NH—, —CH₂NH—, —NHCH₂—, —N(CH₃)CH₂—, —CH₂N(CH₃)—, —CO—, —COCH₂—, —NHCO—, —CH₂NHCO—, —CONHCH₂—, —NHCOCH₂—, —CONH— or —CH₂CONH—;
L_{W1} and L_{W2} are each independently 3- to 11-membered cycloalkyl, heterocyclyl, aryl or heteroaryl optionally substituted with 1 to 3 C₁₋₃alkyl or OH; and
L_{U} and L_{P} are connected to ULM and PTM through [symbol] , respectively.

2. The compound according to claim 1, wherein Formula II is the following Formula II-1: 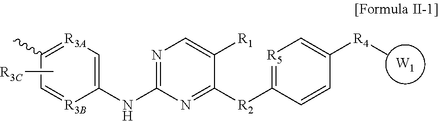
wherein:
R₁ is halo;
R₂ is —NH— or —O—;
R_{3A} and R_{3B} are each independently —CH— or —N—;
R_{3C} is H, halo or —OC₁₋₃alkyl;
R₄ is —CONH—, —CON(CH₃)— or —NHCO—;
R₅ is CH or N; 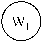
is cyclohexyl or phenyl optionally substituted with 1 to 3 halo; and [symbol]
is a covalent bond connected to Linker.

3. The compound according to claim 1, wherein Formula II is the following Formula II-2: 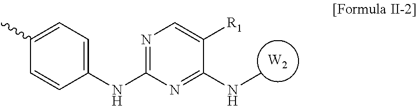
wherein:
R₁ is halo; and 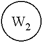
is phenyl or pyrazolyl optionally substituted with halo or C₁₋₃alkyl.

4. The compound according to claim 1, wherein Formula III-1 is the following Formula III-1A or III-1B: 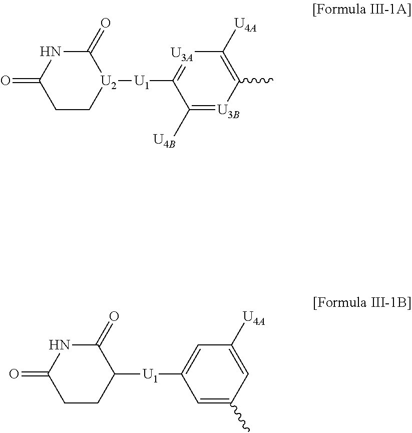
wherein:
U₁ is a covalent bond, —NH—, —N(CH₃)—, —NHCO— or —CONH—;
U₂ is CH or N;
U_{3A} and U_{3B} are each independently CH or N; and
U_{4A} and U_{4B} are each independently H, halo, C₁-3alkyl or OC₁₋₃alkyl.

5. The compound according to claim 1, wherein Formula III-2A or III-2B is the following Formula III-2C or Formula III-2D: 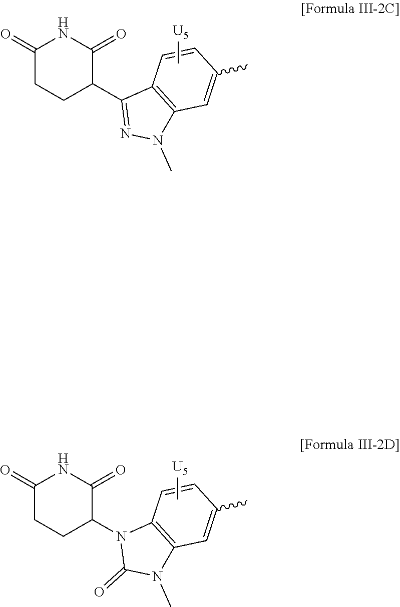
wherein U₅ is H or halo.

6. The compound according to claim 1, wherein L_{U} is a covalent bond or —CC—; and L_{P} is —CH₂O—, —N(CH₃)CH₂—, —CO—, —COCH₂—, —NHCO—, —CH₂NHCO— or —CONH—.

7. The compound according to claim 1, wherein L_{M} is a covalent bond, —CH₂—, —CH₂CH₂—, —CH(CH₃)CH₂—, —CH₂CH(CH₃)—, —CH₂CH(CH₂CH₃)—, —COCH₂—, —CH₂CO—, —CH₂N(CH₃)—, —CH₂CH₂CH₂—, —CH₂CH₂CO—, —CH₂N(CH₃)CH₂— or —CH₂CH₂N(CH₃)—.

8. The compound according to claim 1, wherein L_{W1} and L_{W2} are each independently the following Formula IV-1, IV-2 or IV-3: 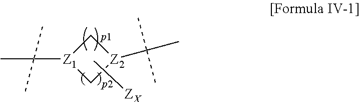
wherein:
Z₁ and Z₂ are each independently CH or N;
Z_{X} is H, CH₃ or OH;
p₁ is 0, 1 or 2; and
p₂ is 1, 2 or 3; 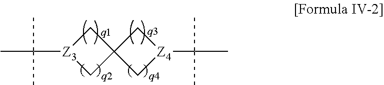
wherein:
Z₃ and Z₄ are each independently CH or N;
q₁ and q₃ are each independently 0, 1 or 2; and
q₂ and q₄ are each independently 1 or 2; 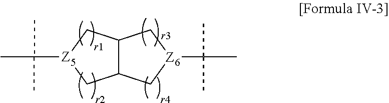
wherein:
Z₅ and Z₆ are each independently CH or N; and
r₁ to r₄ are each independently 0 or 1.

9. The compound according to claim 1, wherein the compound induces AURKA protein degradation.

10. A pharmaceutical composition comprising the compound according to claim 1, and at least one pharmaceutically acceptable carrier.

11. A method, comprising using the pharmaceutical composition according to claim 10 for treating an AURKA-related disease.
